(19) United States
(12) Patent Application Publication
(10) Pub. No.: US 2025/0034551 A1
(43) Pub. Date: Jan. 30, 2025

(54) METHODS AND SYSTEMS FOR HIGH-THROUGHPUT BIOCHEMICAL SCREENS

(71) Applicants: THINK BIOSCIENCE, INC., Boulder, CO (US); THE REGENTS OF THE UNIVERSITY OF COLORADO, A BODY CORPORATE, Denver, CO (US)

(72) Inventors: Nolan O'CONNOR, Denver, CO (US); Andrew MARKLEY, Boulder, CO (US); Hannah EDSTROM, Boulder, CO (US); Jerome Michael FOX, Boulder, CO (US); Tommaso Antonio FODERARO, Broomfield, CO (US); Ankur Kulshreshtha SARKAR, Boulder, CO (US); Matthew TRAYLOR, Boulder, CO (US); Levi Daniel KRAMER, Boulder, CO (US); Gregory DONOVAN, Denver, CO (US)

(21) Appl. No.: 18/707,093

(22) PCT Filed: Nov. 3, 2022
(86) PCT No.: PCT/US2022/079253

§ 371 Date: May 2, 2024

Related U.S. Application Data
(60) Provisional application No. 63/274,988, filed on Nov. 3, 2021.
(60) Provisional application No. 63/281,023, filed on Nov. 18, 2021.
(60) Provisional application No. 63/318,302, filed on Mar. 9, 2022.
(60) Provisional application No. 63/397,780, filed on Aug. 12, 2022.

(51) Int. Cl. C12N 15/10; C12N 9/12; C12N 9/88; C12Q 1/6853; C12Q 1/6869; G01N 33/50

(52) U.S. Cl. CPC C12N 15/1055; C12N 9/12; C12N 9/88; C12Q 1/6853; C12Q 1/6869; G01N 33/5023; C12Q 2600/158; C12Y 207/10; C12Y 402/03017; C12Y 402/03024; C12Y 402/03038; C12Y 402/03056

(57) ABSTRACT

Provided are high-throughput screens for biologically active modulators of a target enzyme that mimic or recreate natural processes of diversification and selection. In some embodiments, the platform comprises one or more expression systems including without limitation (i) a two-hybrid system that, when expressed in a cell, links survival of the cell to the modulation of a therapeutic target, and (ii) a metabolic system that enables the biosynthesis of structurally varied modulators of the therapeutic agent.

### CROSS-REFERENCE

This application claims the benefit of U.S. Provisional Application No. 63/274,988, filed Nov. 3, 2021, U.S. Provisional Application No. 63/281,023, filed Nov. 18, 2021, U.S. Provisional Application No. 63/318,302, filed Mar. 9, 2022, and U.S. Provisional Application No. 63/397,780, filed Aug. 12, 2022, each of which is incorporated herein by reference in its entirety.

### STATEMENT REGARDING FEDERALLY SPONSORED RESEARCH OR DEVELOPMENT

This invention was made with Government support under Grant Nos. 2030347 and 1750244 awarded by the National Science Foundation, and Grant No. 1R35GM143089 awarded by the National Institutes of Health. The Government has certain rights to this invention.

### INCORPORATION BY REFERENCE OF SEQUENCE LISTING

The present application is being filed along with a Sequence Listing in electronic format. The Sequence Listing is provided as a file entitled 57123_703_601.xml, created Nov. 2, 2022, which is 363,456 bytes in size. The information in the electronic format of the Sequence Listing is incorporated by reference in its entirety.

### BACKGROUND

Natural products-molecules produced by plants, microbes, and other living things have been important sources of medicine throughout human history. From herbal remedies to carefully formulated therapeutics, the treatments for many illnesses, including infectious diseases, cancers, and metabolic disorders, have their origins in the natural world. Despite the ubiquity of natural products in modern medicine, the discovery of new natural compounds with therapeutically relevant activities is hampered by their limited abundance and synthetic complexity.

From 1981 to 2014, nearly 50% of all medicines approved for use in the United States by the U.S. Food and Drug Administration (FDA) were natural products, their derivatives, or molecules modeled after them. Of the 459 medicines deemed essential by the World Health Organization (WHO), 202 have natural sources or are derived from naturally-occurring compounds. The tendency for natural products to exert therapeutic effects is often attributed to their origins: molecules made in biological systems are more likely to be biologically active. Considering their important role and proclivity for success in medicine, scientists have exhaustively searched for such molecules.

Terpenoids make up a particularly interesting class of medicinally relevant molecules. These compounds comprise the largest family of natural products (with over 95,000 known structures to date) and account for at least 100 medicines. Produced in all kingdoms of life, this family of compounds is produced, primarily, by terpene synthases. Classically, these enzymes act on prenyl diphosphate substrates of varying lengths, although a small number of synthases have been reported to accept additional substrates. These substrates are produced by prenyltransferases, which condense the five-carbon precursors isoprenyl pyrophosphate (IPP) and dimethylallyl pyrophosphate (DMAPP) into progressively longer diphosphate molecules. From minimally diverse starting materials, terpene synthases can produce—often with remarkable chemoselectivity—a wide range of linear and cyclic structures. Further diversification of these structures is completed by tailoring enzymes, such as cytochrome P450s or dehydrogenases, which introduce heteroatoms and other functional moieties. In nature, functionalized terpenoids play important roles in ecological defense and communication; in medicine, they serve as anti-cancer, anti-inflammatory, and hormone therapies.

Like most medicines, terpenoid-based drugs have often been discovered through serendipity. For example, paclitaxel, an important anti-cancer drug, was discovered in the 1960s by screening over 30,000 extracts of plant/animal material. In modern times, systematic discovery efforts have relied on high-throughput screening (HTS) approaches. HTS typically requires assays that can be miniaturized (<100 μL) and run in parallel with 96-, 348-, or 1536-well plates and liquid handling robotics. While HTS has yielded many important successes, it has several limitations. First, HTS requires highly specialized equipment, development of suitable assays, and infrastructure to efficiently complete experiments. HTS centers, where screens can be carried out at dedicated laboratories with the necessary instruments and expertise, are becoming more common, but screening costs can approach or exceed $1.00/well, leading to costs in the hundreds of thousands of dollars for comprehensive library screens. Additionally, the molecules in natural product libraries are obtained from biological material (e.g., plant matter, soil samples, coral reefs, etc.). Acquiring these samples often requires significant resources and existing sampling strategies have yielded fewer and fewer novel compounds over time. Diversity-oriented chemical synthesis has successfully produced libraries of many compound classes, but this strategy has been limited to only a few terpenoid families that represent a small fraction of what can be found in nature.

Early achievements in heterologous biosynthesis of terpenoids laid the foundation for subsequent microbial production systems by elucidating general principles for heterologous terpene biosynthesis and functionalization (e.g. the design of precursor pathways and the effects of enzyme expression levels and solubility) and by developing strategies and tools for carrying out this work (e.g. optimized combinatorial screening, analytical methods, biosensors for rapid production readouts). These efforts, however, spanned nearly a decade, resulting in high-level production of only a handful of terpenoids (most of which were already known to have medicinal value)—this rate of productivity is incompatible with pharmaceutical discovery, which requires efficient access to many different molecules.

Combinatorial biosynthesis has emerged as an effective approach for producing structurally varied terpenoids. This strategy exploits the inherent promiscuity of certain enzymes (e.g., their ability to act on more than one substrate) by replacing their genes in a biosynthetic pathway with homologs. Often, these homologs carry out different chemical reactions on the same substrate, yielding different metabolites with minimal pathway manipulation. Combinatorial methods are well-suited for producing diverse terpenoids, as their biosynthetic pathways are inherently modular: most unfunctionalized terpenes can be produced by the activity of just two enzyme classes—prenyltransferases and terpene synthases—acting on IPP and dimethylallyl pyrophosphate (DMAPP).

Despite important achievements in synthetic biology, the use of engineered microbes in high-throughput discovery campaigns remains challenging. Microbial production systems have seen rapid development in the past decade but advances in efficient functional characterization of biosynthetic compounds have been lacking. Today, a scientist wanting to screen microbially synthesized terpenoids for activity against a disease-relevant enzyme would have to purify them from cell culture in milligram-scale quantities to identify compounds with a desired activity (which may also require protein purification and assay development). This work can be laborious and, because purification is often not parallelizable, greatly reduces screening throughput.

### SUMMARY

Aspects disclosed herein provide methods of performing multiplexed discovery of bioactive molecules that modulate activity of a target enzyme, the methods comprising: (a) providing a plurality of cells; (b) introducing into each of the plurality of cells a synthetic genetically-encoded system that links expression of a gene of interest to biosynthesis of a bioactive molecule by a cell of the plurality of cells, wherein the synthetic genetically-encoded system encodes: the target enzyme, a gene of interest, a synthase of the bioactive molecule, a ligand, and a receptor specific to the ligand under conditions sufficient for binding of the ligand to the receptor to produce a ligand-receptor pair, wherein the ligand-receptor pair activates transcription of the gene of interest; (c) performing multiplexed sequencing of the plurality of cells; and (d) identifying a subset of the plurality of cells in which the expression of the gene of interest is increased relative to a reference expression level, wherein the reference expression level is obtained from an otherwise identical reference cell that does not comprise a metabolic pathway that produces the bioactive molecule, the ligand or the receptor. In some embodiments, the expression of the gene of interest is increased relative to the reference expression level when the bioactive molecule is present in the cell at concentrations sufficient to modulate the activity of the target enzyme. In some embodiments, modulation of the target enzyme by the bioactive molecule disrupts binding between the receptor and the ligand, thereby reducing transcriptional activation of the gene of interest. In some embodiments, the binding of the ligand to the receptor is phosphorylation dependent. In some embodiments, the plurality of cells are prokaryotic cells. In some embodiments, the prokaryotic cells comprise bacterial cells. In some embodiments, the bioactive molecule comprises a terpenoid. In some embodiments, the target enzyme comprises a proteolytic enzyme, a phosphatase, or a kinase. In some embodiments, the phosphatase comprises a tyrosine phosphatase. In some embodiments, the kinase comprises a tyrosine kinase. In some embodiments, the exogenous genetically-encoded system comprises a two-hybrid system encoding the target enzyme, the gene of interest, the ligand, and the receptor. In some embodiments, the synthase comprises a terpene synthase or a nonribosomal peptide synthetase. In some embodiments, the terpene synthase comprises γ-humulene synthase (GHS), amorphadiene synthase (ADS), α-bisabolene synthase (ABS), or taxadiene synthase (TXS). In some embodiments, the terpene synthase comprises an amino acid sequence that is greater than or equal to about 90% identical to any one of SEQ ID NO: 4, 7, 9, 11, 13, 15, 17, 19, or 23. In some embodiments, the ligand comprises a kinase substrate that binds to the receptor in a phosphorylated state, and the receptor comprises a phosphorylated protein binding domain that binds to the kinase substrate when the kinase substrate is phosphorylated. In some embodiments, the ligand is coupled to a subunit of RNA polymerase and the receptor is coupled to a DNA binding domain, or the ligand is coupled to the DNA binding domain and the receptor is coupled to the omega subunit of the RNA polymerase. In some embodiments, the gene of interest encodes a reporter polypeptide comprising a luciferase enzyme, a fluorescent polypeptide, secreted alkaline phosphatase, ß-galactosidase levansucrase, chloramphenicol acetyltransferase (CAT), or a protein that confers antibiotic resistance. In some embodiments, the gene of interest encodes a polymerizing enzyme that, when expressed, binds to a promoter operably linked to a gene encoding the reporter polypeptide to drive expression of the reporter polypeptide. In some embodiments, the expression of the reporter polypeptide from the gene is greater than an expression of the reporter polypeptide if it were encoded by the gene of interest. In some embodiments, the expression of the reporter polypeptide is greater by more than or equal to about 2-fold. In some embodiments, the genetically-encoded system further encodes a metabolic pathway for biosynthesis of the bioactive molecule. In some embodiments, the metabolic pathway is an isoprenoid pathway. In some embodiments, the isoprenoid pathway comprises a mevalonate pathway, a methylerthritol 4-phosphate (MEP) pathway, a deoxyxylulose 5-phosphate (DXP) pathway, or an isopentenol utilization (IUP) pathway. In some embodiments, the multiplex sequencing comprises long read sequencing. In some embodiments, the synthetic genetically-encoded system comprises one or more molecular barcode sequences that uniquely identifies the target enzyme, the synthase, or a combination thereof. In some embodiments, the multiplex sequencing further comprises performing demultiplexing, thereby assigning each of the one or more molecular barcodes with the target enzyme, the synthase, or the combination thereof, for each cell of the subset of the plurality of cells. In some embodiments, the method further comprises performing multiplexed sequencing of the plurality of cells prior to introducing in (b), wherein the identifying in (d) comprises detecting enrichment of the gene of interest following the introducing in (b).

Aspects disclosed herein provide systems of linking expression of a gene of interest to biosynthesis by the cell of a bioactive molecule that modulates activity of a target enzyme, the systems comprising: one or more nucleic acid molecules encoding a genetically-encoded system that, when expressed in a cell, links expression of a gene of interest to biosynthesis by the cell of a bioactive molecule that modulates activity of a target enzyme, wherein the genetically-encoded system comprises: the target enzyme; a synthase of the bioactive molecule; a ligand; and a receptor specific to the ligand, wherein, (i) the ligand is coupled to a subunit of RNA polymerase and the receptor is coupled to a DNA binding protein, or (ii) the ligand is coupled to the DNA binding protein and the receptor is coupled to the subunit of RNA polymerase, and wherein the one or more nucleic acid molecules comprises: one or more adaptor molecules comprising a sequencing primer binding site; the gene of interest; and a transcription initiation site for the gene of interest comprising: a binding site for the DNA binding protein; and a promoter sequence comprising a binding site for the RNA polymerase. In some embodiments, the system further comprises the cell comprising the one or more nucleic acid molecules. In some embodiments, the cell is a prokaryotic cell. In some embodiments, the prokaryotic cell comprises a bacterial cell. In some embodiments, the cell is isolated. In some embodiments, the bioactive molecule comprises a terpenoid. In some embodiments, the target enzyme comprises a proteolytic enzyme, a phosphatase, or a kinase. In some embodiments, the phosphatase comprises a tyrosine phosphatase. In some embodiments, the kinase comprises a tyrosine kinase. In some embodiments, the subunit of the RNA polymerase comprises an omega subunit of the RNA polymerase. In some embodiments, the exogenous genetically-encoded system comprises a two-hybrid system encoding the target enzyme, the ligand, the receptor, and the gene of interest. In some embodiments, the synthase comprises a terpene synthase or a nonribosomal peptide synthetase. In some embodiments, the terpene synthase comprises γ-humulene synthase (GHS), amorphadiene synthase (ADS), α-bisabolene synthase (ABS), or taxadiene synthase (TXS). In some embodiments, the terpene synthase comprises an amino acid sequence that is greater than or equal to about 90% identical to any one of SEQ ID NO: 4, 7, 9, 11, 13, 15, 17, 19, or 23. In some embodiments, the ligand comprises a kinase substrate that binds to the receptor in a phosphorylated state, and the receptor comprises a phosphorylated protein binding domain that binds to the kinase substrate when the kinase substrate is phosphorylated. In some embodiments, the ligand is coupled to a subunit of RNA polymerase and the receptor is coupled to a DNA binding domain, or the ligand is coupled to the DNA binding domain and the receptor is coupled to the subunit of the RNA polymerase. In some embodiments, the gene of interest encodes a reporter polypeptide comprising a luciferase enzyme, a fluorescent polypeptide, secreted alkaline phosphatase, β-galactosidase levansucrase, chloramphenicol acetyltransferase (CAT), or a protein that confers antibiotic resistance. In some embodiments, the gene of interest encodes a modulator protein that is operably linked to a gene encoding the reporter polypeptide, wherein the modulator protein activates or represses expression of the reporter polypeptide. In some embodiments, the one or more adaptor molecules comprises one or more molecular barcode sequences unique to the target enzyme, the synthase, or the combination thereof. In some embodiments, the genetically-encoded system further encodes a metabolic pathway for biosynthesis of the bioactive molecule. In some embodiments, the metabolic pathway is an isoprenoid pathway. In some embodiments, the isoprenoid pathway comprises a mevalonate pathway, a methylerthritol 4-phosphate (MEP) pathway, a deoxyxylulose 5-phosphate (DXP) pathway, or an isopentenol utilization (IUP) pathway. In some embodiments, the one or more adaptor molecules further comprises another barcode sequence unique to the metabolic pathway.

Aspects disclosed herein provide methods of determining a presence of a bioactive molecule that modulates activity of a target enzyme, the methods comprising: (a) introducing into a cell a synthetic genetically-encoded system that links expression of a gene of interest to biosynthesis of the bioactive molecule by the cell, wherein the synthetic genetically-encoded system encodes: the target enzyme, a gene of interest encoding modulatory protein that modulates expression of a reporter polypeptide, the reporter polypeptide, a synthase of the bioactive molecule, a ligand, and a receptor specific to the ligand under conditions sufficient for binding of the ligand to the receptor to form a ligand-receptor pair, wherein the ligand-receptor pair activates transcription of the gene of interest; (b) measuring the expression of the reporter polypeptide; and (c) determining the presence of the bioactive molecule in the cell if the expression of the reporter polypeptide is increased or decreased relative to a reference expression level obtained from an otherwise identical reference cell that does not comprise a functional metabolic pathway that produces the bioactive molecule, the ligand or the receptor. In some embodiments, the expression of the reporter polypeptide is increased relative to the reference expression level when the bioactive molecule is present in the cell at concentrations sufficient to modulate the activity of the target enzyme. In some embodiments, the modulatory protein comprises a polymerizing enzyme that activates transcription of the reporter polypeptide. In some embodiments, the expression of the reporter polypeptide is decreased relative to the reference expression level when the bioactive molecule is present in the cell at concentrations sufficient to modulate the activity of the target enzyme. In some embodiments, the modulatory protein comprises a transcriptional repressor that represses transcription of the reporter polypeptide. In some embodiments, modulation of the target enzyme by the bioactive molecule disrupts binding between the receptor and the ligand, thereby reducing transcriptional activation of the gene of interest. In some embodiments, the binding of the ligand to the receptor is phosphorylation dependent. In some embodiments, cell is a prokaryotic cell. In some embodiments, the prokaryotic cell is a bacterial cell. In some embodiments, the bioactive molecule comprises a terpenoid. In some embodiments, the target enzyme comprises a proteolytic enzyme, a phosphatase, or a kinase. In some embodiments, the phosphatase comprises a tyrosine phosphatase. In some embodiments, the kinase comprises a tyrosine kinase. In some embodiments, the exogenous genetically-encoded system comprises a two-hybrid system encoding the target enzyme, the ligand, the receptor, and the gene of interest. In some embodiments, the synthase comprises a terpene synthase or a nonribosomal peptide synthetase. In some embodiments, the terpene synthase comprises γ-humulene synthase (GHS), amorphadiene synthase (ADS), α-bisabolene synthase (ABS), or taxadiene synthase (TXS). In some embodiments, the terpene synthase comprises an amino acid sequence that is greater than or equal to about 90% identical to any one of SEQ ID NO: 4, 7, 9, 11, 13, 15, 17, 19, or 23. In some embodiments, the ligand comprises a kinase substrate that binds to the receptor in a phosphorylated state, and the receptor comprises a phosphorylated protein binding domain that binds to the kinase substrate when the kinase substrate is phosphorylated. In some embodiments, the ligand is coupled to a subunit of RNA polymerase and the receptor is coupled to a DNA binding domain, or the ligand is coupled to the DNA binding domain and the receptor is coupled to the subunit of the RNA polymerase. In some embodiments, the reporter polypeptide comprises a luciferase enzyme, a fluorescent polypeptide, secreted alkaline phosphatase, ß-galactosidase levansucrase, chloramphenicol acetyltransferase (CAT), or a protein that confers antibiotic resistance. In some embodiments, the genetically-encoded system further encodes a metabolic pathway for biosynthesis of the bioactive molecule. In some embodiments, the metabolic pathway is an isoprenoid pathway. In some embodiments, the isoprenoid pathway comprises a mevalonate pathway, a methylerthritol 4-phosphate (MEP) pathway, a deoxyxylulose 5-phosphate (DXP) pathway, or an isopentenol utilization (IUP) pathway.

Aspects disclosed herein provide systems of linking expression of a gene of interest to biosynthesis by the cell of a bioactive molecule that modulates activity of a target enzyme, the systems comprising: one or more nucleic acid molecules encoding a genetically-encoded system that, when expressed in a cell, links expression of a gene of interest to biosynthesis by the cell of a bioactive molecule that modulates activity of a target enzyme, wherein the genetically-encoded system comprises: a reporter polypeptide; the target enzyme; a synthase of the bioactive molecule; a ligand; and a receptor specific to the ligand, wherein, (i) the ligand is coupled to a subunit of RNA polymerase and the receptor is coupled to a DNA binding protein, or (ii) the ligand is coupled to the DNA binding protein and the receptor is coupled to the subunit of RNA polymerase, and wherein the one or more nucleic acid molecules comprises: the gene of interest, wherein the gene of interest encodes a modulator protein configured to activate transcription or repress transcription of the reporter polypeptide; and a transcription initiation site for the gene of interest comprising: a binding site for the DNA binding protein; and a promoter sequence comprising a binding site for the RNA polymerase. In some embodiments, the modulatory protein comprises a polymerizing enzyme that activates transcription of the reporter polypeptide. In some embodiments, the modulatory protein comprises a transcriptional repressor that represses transcription of the reporter polypeptide. In some embodiments, the system further comprises the cell comprising the one or more nucleic acid molecules. In some embodiments, the cell is a prokaryotic cell. In some embodiments, the prokaryotic cell comprises a bacterial cell. In some embodiments, the cell is isolated. In some embodiments, the bioactive molecule comprises a terpenoid. In some embodiments, the target enzyme comprises a proteolytic enzyme, a phosphatase, or a kinase. In some embodiments, the phosphatase comprises a tyrosine phosphatase. In some embodiments, the kinase comprises a tyrosine kinase. In some embodiments, the subunit of the RNA polymerase comprises an omega subunit of the RNA polymerase. In some embodiments, the exogenous genetically-encoded system comprises a two-hybrid system encoding the target enzyme, the ligand, the receptor, and the gene of interest. In some embodiments, the synthase comprises a terpene synthase or a nonribosomal peptide synthetase. In some embodiments, the terpene synthase comprises γ-humulene synthase (GHS), amorphadiene synthase (ADS), α-bisabolene synthase (ABS), or taxadiene synthase (TXS). In some embodiments, the terpene synthase comprises an amino acid sequence that is greater than or equal to about 90% identical to any one of SEQ ID NO: 4, 7, 9, 11, 13, 15, 17, 19, or 23. In some embodiments, the ligand comprises a kinase substrate that binds to the receptor in a phosphorylated state, and the receptor comprises a phosphorylated protein binding domain that binds to the kinase substrate when the kinase substrate is phosphorylated. In some embodiments, the ligand is coupled to a subunit of RNA polymerase and the receptor is coupled to a DNA binding domain, or the ligand is coupled to the DNA binding domain and the receptor is coupled to the subunit of the RNA polymerase. In some embodiments, the reporter polypeptide comprises a luciferase enzyme, a fluorescent polypeptide, secreted alkaline phosphatase, ß-galactosidase levansucrase, chloramphenicol acetyltransferase (CAT), or a protein that confers antibiotic resistance. In some embodiments, the genetically-encoded system further encodes a metabolic pathway for biosynthesis of the bioactive molecule. In some embodiments, the metabolic pathway is an isoprenoid pathway. In some embodiments, the isoprenoid pathway comprises a mevalonate pathway, a methylerthritol 4-phosphate (MEP) pathway, a deoxyxylulose 5-phosphate (DXP) pathway, or an isopentenol utilization (IUP) pathway. In some embodiments, the one or more nucleic acid molecules further comprises one or more barcode sequences unique to the metabolic pathway. In some embodiments, the one or more nucleic acid molecules further comprises one or more barcode sequences unique to the synthase, the target enzyme or a combination thereof.

Aspects disclosed herein provide methods of determining a presence of a bioactive molecule that modulates the activity of a target enzyme, the methods comprising: (a) introducing into a cell a synthetic genetically-encoded system that links expression of a gene of interest to biosynthesis of the bioactive molecule by the cell, wherein the synthetic genetically-encoded system encodes the target enzyme comprising: a proteolytic enzyme, the gene of interest, a synthase of the bioactive molecule, a ligand, and a receptor specific to the ligand under conditions sufficient for binding of the ligand to the receptor to form a ligand-receptor pair, wherein the ligand-receptor pair comprises a cleavage site recognized by the proteolytic enzyme, and activates transcription of the gene of interest; (b) measuring the expression of the gene of interest; and (c) determining the presence of the bioactive molecule in the cell if the expression of the gene of interest is increased relative to a reference expression level obtained from an otherwise identical reference cell that does not comprise a metabolic pathway that produces the bioactive molecule, the ligand or the receptor. In some embodiments, the expression of the gene of interest is increased relative to the reference expression level when the bioactive molecule is present in the cell at concentrations sufficient to modulate the activity of the target enzyme. In some embodiments, modulation of the target enzyme by the bioactive molecule disrupts binding between the receptor and the ligand, thereby reducing transcriptional activation of the gene of interest. In some embodiments, the binding of the ligand to the receptor is phosphorylation dependent. In some embodiments, the cell is a prokaryotic cell. In some embodiments, the prokaryotic cell comprises a bacterial cell. In some embodiments, the bioactive molecule comprises a terpenoid. In some embodiments, the proteolytic enzyme comprises a viral proteolytic enzyme. In some embodiments, the viral proteolytic enzyme comprises 3CL protease (3CLpro) of severe acute respiratory syndrome coronavirus 2 (SARS-COV-2), NS2B/NS3 protease of West Nile Virus, or papain-like protease (PLpro) of SARS-COV-2. In some embodiments, the proteolytic enzyme comprises a ubiquitin specific protease. In some embodiments, the ubiquitin specific protease is ubiquitin specific protease 7 (USP7). In some embodiments, the synthetic genetically-encoded system comprises a two-hybrid system encoding the target enzyme, the ligand, the receptor, and the gene of interest. In some embodiments, the synthase comprises a terpene synthase or a nonribosomal peptide synthetase. In some embodiments, the terpene synthase comprises γ-humulene synthase (GHS), amorphadiene synthase (ADS), α-bisabolene synthase (ABS), or taxadiene synthase (TXS). In some embodiments, the terpene synthase comprises an amino acid sequence that is greater than or equal to about 90% identical to any one of SEQ ID NO: 4, 7, 9, 11, 13, 15, 17, 19, or 23. In some embodiments, the ligand comprises a kinase substrate that binds to the receptor in a phosphorylated state, and the receptor comprises a phosphorylated protein binding domain that binds to the kinase substrate when the kinase substrate is phosphorylated. In some embodiments, the ligand is coupled to a subunit of RNA polymerase and the receptor is coupled to a DNA binding domain, or the ligand is coupled to the DNA binding domain and the receptor is coupled to the subunit of the RNA polymerase. In some embodiments, the ligand comprises a linker coupled to the subunit of RNA polymerase. In some embodiments, the receptor comprises a linker coupled to the subunit of RNA polymerase. In some embodiments, the linker comprises the cleavage site recognized by the proteolytic enzyme. In some embodiments, the cleavage site comprises an amino acid sequence comprising AVLQSGFR (SEQ ID NO: 1), KARVLAEAM (SEQ ID NO: 2), LRGG (SEQ ID NO: 3), or SEQ ID NO: 25. In some embodiments, the linker comprises one or more alanine residues flanking the cleavage site. In some embodiments, the gene of interest encodes a reporter polypeptide comprising a luciferase enzyme, a fluorescent polypeptide, secreted alkaline phosphatase, ß-galactosidase levansucrase, chloramphenicol acetyltransferase (CAT), or a protein that confers antibiotic resistance. In some embodiments, the gene of interest encodes a modulator protein that is operably linked to a gene encoding a reporter polypeptide, wherein the modulator protein activates or represses expression of the reporter polypeptide. In some embodiments, the expression of the reporter polypeptide is greater than an expression of the reporter polypeptide if the reporter polypeptide were encoded by the gene of interest. In some embodiments, the expression of the reporter polypeptide is greater by more than or equal to about 2-fold. In some embodiments, the genetically-encoded system further encodes a metabolic pathway for biosynthesis of the bioactive molecule. In some embodiments, the metabolic pathway is an isoprenoid pathway. In some embodiments, the isoprenoid pathway comprises a mevalonate pathway, a methylerthritol 4-phosphate (MEP) pathway, a deoxyxylulose 5-phosphate (DXP) pathway, or an isopentenol utilization (IUP) pathway.

Aspects disclosed herein provide systems of linking expression of a gene of interest to biosynthesis by the cell of a bioactive molecule that modulates activity of a target enzyme, the systems comprising: one or more nucleic acid molecules encoding a genetically-encoded system that, when expressed in a cell, links expression of a gene of interest to biosynthesis by the cell of a bioactive molecule that modulates activity of a target enzyme, wherein the genetically-encoded system comprises: a metabolic pathway for biosynthesis of the bioactive molecule; the target enzyme, wherein the target enzyme comprises a proteolytic enzyme; a synthase of the bioactive molecule; a ligand; and a receptor specific to the ligand, wherein (i) the ligand is coupled to a subunit of RNA polymerase and the receptor is coupled to a DNA binding protein, or (ii) the ligand is coupled to the DNA binding protein and the receptor is coupled to the subunit of RNA polymerase; wherein the receptor or the ligand comprise a cleavage site recognized by the proteolytic enzyme; and wherein the one or more nucleic acid molecules comprises: the gene of interest; and a transcription initiation site for the gene of interest comprising: a binding site for the DNA binding protein; and a promoter sequence comprising a binding site for the RNA polymerase. In some embodiments, the system further comprises the cell comprising the one or more nucleic acid molecules. In some embodiments, the cell is a prokaryotic cell. In some embodiments, the prokaryotic cell comprises a bacterial cell. In some embodiments, the cell is isolated. In some embodiments, the bioactive molecule comprises a terpenoid. In some embodiments, the proteolytic enzyme comprises a viral proteolytic enzyme. In some embodiments, the viral proteolytic enzyme comprises 3CL protease (3CLpro) of severe acute respiratory syndrome coronavirus 2 (SARS-COV-2), NS2B/NS3 protease of West Nile Virus, or papain-like protease (PLpro) of SARS-COV-2. In some embodiments, the proteolytic enzyme comprises a ubiquitin specific protease. In some embodiments, the ubiquitin specific protease is ubiquitin specific protease 7 (USP7). In some embodiments, the subunit of the RNA polymerase comprises an omega subunit of the RNA polymerase. In some embodiments, the synthetic genetically-encoded system comprises a two-hybrid system encoding the target enzyme, the ligand, the receptor, and the gene of interest. In some embodiments, the synthase comprises a terpene synthase or a nonribosomal peptide synthetase. In some embodiments, the terpene synthase comprises γ-humulene synthase (GHS), amorphadiene synthase (ADS), α-bisabolene synthase (ABS), or taxadiene synthase (TXS). In some embodiments, the terpene synthase comprises an amino acid sequence that is greater than or equal to about 90% identical to any one of SEQ ID NO: 4, 7, 9, 11, 13, 15, 17, 19, or 23. In some embodiments, the ligand comprises a kinase substrate that binds to the receptor in a phosphorylated state, and the receptor comprises a phosphorylated protein binding domain that binds to the kinase substrate when the kinase substrate is phosphorylated. In some embodiments, the ligand is coupled to a subunit of RNA polymerase and the receptor is coupled to a DNA binding domain, or the ligand is coupled to the DNA binding domain and the receptor is coupled to the subunit of the RNA polymerase. In some embodiments, the ligand comprises a linker coupled to the subunit of RNA polymerase. In some embodiments, the receptor comprises a linker coupled to the subunit of RNA polymerase. In some embodiments, the linker comprises the cleavage site recognized by the proteolytic enzyme. In some embodiments, the cleavage site comprises an amino acid sequence comprising AVLQSGFR (SEQ ID NO: 1), KARVLAEAM (SEQ ID NO: 2), LRGG (SEQ ID NO: 3), or SEQ ID NO: 25. In some embodiments, the linker comprises one or more alanine residues flanking the cleavage site. In some embodiments, the reporter polypeptide comprises a luciferase enzyme, a fluorescent polypeptide, secreted alkaline phosphatase, ß-galactosidase levansucrase, chloramphenicol acetyltransferase (CAT), or a protein that confers antibiotic resistance. In some embodiments, the genetically-encoded system further encodes a metabolic pathway for biosynthesis of the bioactive molecule. In some embodiments, the metabolic pathway is an isoprenoid pathway. In some embodiments, the isoprenoid pathway comprises a mevalonate pathway, a methylerthritol 4-phosphate (MEP) pathway, a deoxyxylulose 5-phosphate (DXP) pathway, or an isopentenol utilization (IUP) pathway. In some embodiments, the one or more nucleic acid molecules further comprises one or more barcode sequences unique to the metabolic pathway. In some embodiments, the one or more nucleic acid molecules further comprises one or more barcode sequences unique to the synthase, the target enzyme or a combination thereof.

Aspects disclosed herein provide systems for identifying a protease modulator, the systems comprising: a first nucleic acid sequence encoding a phosphorylated tyrosine binding domain, wherein the phosphorylated tyrosine binding domain optionally comprises a Src homology 2 (SH2) domain; a second nucleic acid sequence encoding a repressor element, wherein the repressor element optionally comprises a cI repressor; a third nucleic acid sequence encoding a subunit of a RNA polymerase, wherein the subunit of the RNA polymerase is optionally an omega subunit of the RNA polymerase (RpoZ); a fourth nucleic acid sequence encoding a tyrosine kinase substrate; a fifth nucleic acid sequence encoding a tyrosine kinase; a sixth nucleic acid encoding a target protease; a seventh nucleic acid sequence encoding a protease cleavage site; an eighth nucleic acid encoding an operator for the repressor element, wherein the operator for the repressor element optionally comprises a cI repressor; a ninth nucleic acid sequence comprising a binding site for the RNA polymerase; and a tenth nucleic acid sequence encoding a reporter gene. In some embodiments, the systems further comprise an eleventh nucleic acid sequence encoding Hsp90 co-chaperone Cdc37. In some embodiments, the tyrosine kinase comprises Src kinase. In some embodiments, the first nucleic acid sequence and the second nucleic acid sequence encode the phosphorylated tyrosine binding domain fused with the repressor element. In some embodiments, the third nucleic acid sequence and the fourth nucleic acid sequence encode the subunit of the RNA polymerase fused with the tyrosine kinase substrate. In some embodiments, the protease cleavage site is positioned in a linker region disposed between the subunit of the RNA polymerase and the tyrosine kinase substrate. In some embodiments, the first nucleic acid sequence and the fourth nucleic acid sequence encode the phosphorylated tyrosine binding domain fused with the tyrosine kinase substrate. In some embodiments, the second nucleic acid sequence and the third nucleic acid sequence encode the repressor element fused with the subunit of the RNA polymerase. In some embodiments, a first barcode sequence operably linked to the sixth nucleic acid sequence, wherein the first barcode is sufficient to identify the target protease. In some embodiments, the barcode comprises an index, wherein the index comprises greater than or equal to about 6 contiguous base pairs that are specific to the target protease. In some embodiments, an exogenous nucleic acid encoding a terpene synthase, nonribosomal peptide synthetase, or a combination thereof. In some embodiments, the exogenous nucleic acid encoding the terpene synthase, nonribosomal peptide synthetase, or a combination thereof comprises a second barcode sequence sufficient to identify the terpene synthase. In some embodiments, the second barcode comprises an index, and wherein the index comprises greater than or equal to about 6 contiguous base pairs that are specific to the terpene synthase. In some embodiments, the exogenous nucleic acid further encodes an enzyme configured to catalyze condensation of (i) isopentenyl diphosphate (IPP), (ii) dimethylallyl diphosphate (DMAPP), or (iii) a combination of IPP and DMAPP. In some embodiments, the enzyme comprises a geranylgeranyl diphosphate synthase (GGPPS). In some embodiments, the exogenous nucleic acid further encodes a cytochrome P450 enzyme, a cytochrome P450 reductase enzyme, a cytochrome b5 enzyme, an oxidase enzyme, an acyltransferase enzyme, a glycosyltransferase enzyme, a halogenase, a peroxidase, or any combination thereof. In some embodiments, another exogenous nucleic acid sequence encoding a metabolic pathway for (i) the IPP, (ii) the DMAPP, (iii) the combination of the IPP and the DMAPP, (iv) molecules resulting from the condensation of the IPP, the DMAPP, or the combination of the IPP and the DMAPP, or (v) any combination of (i) to (iv). In some embodiments, the tyrosine kinase substrate comprises a polypeptide, and wherein the polypeptide comprises a tyrosine residue configured to (i) be phosphorylated by the Src kinase, (ii) bind to the SH2 domain when the tyrosine residue is phosphorylated, (iii) bind to the SH2 domain with less binding affinity as compared to the binding affinity between the tyrosine residue and the SH2 domain when the tyrosine residue is dephosphorylated, or (iv) any combination of (i) to (iii). In some embodiments, the tyrosine kinase substrate comprises a substrate domain derived from a hamster polyomavirus middle T antigen (MidT). In some embodiments, the seventh nucleic acid sequence encoding the protease cleavage site comprises an amino acid sequence configured to be hydrolyzed by: (i) the 3CL protease (3CLpro) of severe acute respiratory syndrome coronavirus 2 (SARS-COV-2), (ii) NS2B/NS3 protease of West Nile Virus, (iii) papain-like protease (PLpro) of SARS-COV-2, or (iv) ubiquitin specific protease 7 (USP7). In some embodiments, the amino acid sequence comprises AVLQSGFR (SEQ ID NO: 1). In some embodiments, the amino acid sequence further comprises fewer than or equal to 4 alanine residues on an N-terminus, C-terminus, or combination of the N-terminus and C-terminus of the amino acid sequence. In some embodiments, the seventh nucleic acid sequence encoding the protease cleavage site comprises an amino acid sequence configured to be hydrolyzed by human immunodeficiency virus 1 protease (HIV1pro). In some embodiments, the amino acid sequence comprises KARVLAEAM (SEQ ID NO: 2). In some embodiments, the amino acid sequence further comprises fewer than or equal to 4 alanine residues on an N-terminus, C-terminus, or combination of the N-terminus and C-terminus of the amino acid sequence. In some embodiments a single nucleic acid molecule comprising any combination of the first nucleic acid sequence, the second nucleic acid sequence, the third nucleic acid sequence, the fourth nucleic acid sequence, the fifth nucleic acid sequence, the sixth nucleic acid sequence, the seventh nucleic acid sequence, the eighth nucleic acid sequence, the ninth nucleic acid sequence, and the tenth nucleic acid sequence. In some embodiments, the single nucleic acid molecule is a vector comprising a plasmid vector, a viral vector, a cosmid, an artificial chromosome, a region of a host chromosome, or any combination thereof. In some embodiments, one or more of the first nucleic acid sequence, the second nucleic acid sequence, the third nucleic acid sequence, the fourth nucleic acid sequence, the fifth nucleic acid sequence, the sixth nucleic acid sequence, the seventh nucleic acid sequence, the eighth nucleic acid sequence, the ninth nucleic acid sequence, and the tenth nucleic acid sequence is a vector comprising a plasmid vector, a viral vector, a cosmid, an artificial chromosome, a region of a host chromosome, or any combination thereof. In some embodiments, the reporter gene encodes: a luciferase enzyme; a fluorescent polypeptide; secreted alkaline phosphatase; ß-galactosidase levansucrase; chloramphenicol acetyltransferase (CAT); antibiotic resistance; or a polymerizing enzyme that, when expressed, binds to a promoter operably linked to a gene encoding a detectable polypeptide to drive expression of the detectable polypeptide. In some embodiments, the expression of the detectable polypeptide is greater than an expression of the detectable polypeptide when the gene encoding the detectable polypeptide is included as the reporter gene.

Aspects disclosed herein provide isolated cells comprising systems for identifying a protease modulator, the systems comprising: a first nucleic acid sequence encoding a phosphorylated tyrosine binding domain, wherein the phosphorylated tyrosine binding domain optionally comprises a Src homology 2 (SH2) domain; a second nucleic acid sequence encoding a repressor element, wherein the repressor element optionally comprises a cI repressor; a third nucleic acid sequence encoding a subunit of a RNA polymerase, wherein the subunit of the RNA polymerase is optionally an omega subunit of the RNA polymerase (RpoZ); a fourth nucleic acid sequence encoding a tyrosine kinase substrate; a fifth nucleic acid sequence encoding a tyrosine kinase; a sixth nucleic acid encoding a target protease; a seventh nucleic acid sequence encoding a protease cleavage site; an eighth nucleic acid encoding an operator for the repressor element, wherein the operator for the repressor element optionally comprises a cI repressor; a ninth nucleic acid sequence comprising a binding site for the RNA polymerase; and a tenth nucleic acid sequence encoding a reporter gene. In some embodiments, the isolated cell comprises a prokaryotic cell. In some embodiments, the isolated cell is obtained from a unicellular organism. In some embodiments, the isolated cell comprises a bacterial cell, an algae cell, an archaea cell, a protozoa cell, or a fungal cell. In some embodiments, the fungal cell is a yeast cell. In some embodiments, the bacterial cell is an Escherichia coli (E. coli) cell.

Aspects disclosed herein provide methods of identifying a modulator of the target protease, the methods comprising (a) expressing in a cell an exogenous terpene synthase; (b) introducing into the cell the system of high throughput screening of bioactive molecules that modulate a target enzyme that links the modulation of the target protease with expression of the reporter gene; and (c) measuring expression of the reporter gene in the presence of expression of the terpene synthase, wherein an increased or decreased expression of the reporter gene as compared to a reference expression level indicates a presence of the modulator of the target protease produced by the cell. In some embodiments, expressing in the cell an exogenous nucleic acid sequence encoding a metabolic pathway for (i) isopentenyl diphosphate (IPP), (ii) dimethylallyl diphosphate (DMAPP), (iii) the combination of the IPP and the DMAPP, (iv) molecules resulting from condensation of the IPP, the DMAPP, or the combination of the IPP and the DMAPP, or (v) any combination of (i) to (iv). In some embodiments, the cell further comprises an enzyme configured to catalyze the condensation of (i) the IPP, (ii) the DMAPP, or (iii) a combination of the IPP and the DMAPP. In some embodiments, the enzyme comprises a geranylgeranyl diphosphate synthase (GGPPS). In some embodiments, culturing the cell in a growth cell medium, wherein the growth cell medium comprises glycerol at a concentration comprising less than or equal to about 2% (by volume). In some embodiments, culturing the cell in a growth cell medium, wherein the growth cell medium comprises mevalonate at a concentration comprising less than or equal to about 20 micromolar (mM). In some embodiments, isolating the modulator of the target protease. In some embodiments, the target protease comprises a viral protease. In some embodiments, the viral protease comprises HIV-1 protease (HIV-1Pr) or SARS-COV-2 main protease (3ClPro), (ii) NS2B/NS3 protease (WNV) of West Nile Virus, (iii) papain-like protease (PLpro) of SARS-COV-2, or (iv) Dengue Virus Protease (DVpro). In some embodiments the target protease is a human protease. In some embodiments, the human protease is ubiquitin-specific protease 7 (USP7). In some embodiments, the USP7 is a cancer target. In some embodiments, the introducing of (b) is performed under conditions sufficient to cause the omega subunit of the RNA polymerase to recruit RNA polymerase to the binding site for the RNA polymerase in the absence of a protease, thereby expressing the reporter gene. In some embodiments, the reference expression level is derived from a reference cell expressing the second nucleic acid sequence that is modified such that the tyrosine kinase substrate contains a mutation that inhibits its binding to the SH2 domain. In some embodiments, the reference expression level is derived from a reference cell comprising a modified terpene synthase comprising a mutation that reduces its activity as compared with an otherwise identical terpene synthase that does not have the mutation. In some embodiments, repeating (a) to (c), wherein for each repetition, a new exogenous terpene synthase is used to identify a new modulator of the target protease. In some embodiments, the modulator of the target protease is a terpene.

Aspects disclosed herein provide systems for high throughput screening of bioactive molecules that inhibit a target enzyme, the systems comprising: a first nucleic acid sequence encoding a phosphorylated tyrosine binding domain; a second nucleic acid sequence encoding a repressor element; a third nucleic acid sequence encoding a subunit of RNA polymerase; a fourth nucleic acid sequence encoding a tyrosine kinase substrate; a fifth nucleic acid sequence encoding tyrosine kinase; a sixth nucleic acid encoding the target enzyme, wherein the sixth nucleic acid comprises a barcode sufficient to identify the target enzyme; a seventh nucleic acid encoding an operator for the repressor element; an eighth nucleic acid sequence comprising a binding site for the RNA polymerase; and a ninth nucleic acid sequence encoding a reporter gene. In some embodiments, the phosphorylated tyrosine binding domain comprises a Src homology 2 (SH2) domain. In some embodiments, the repressor element comprises a cI repressor. In some embodiments, the subunit of the RNA polymerase comprises an omega subunit of the RNA polymerase. In some embodiments, the tyrosine kinase comprises Src kinase. In some embodiments, the target enzyme is a tyrosine phosphatase, a protease, or a combination thereof. In some embodiments, the protease is a viral protease. In some embodiments, the protease prevents transcriptional activation by stopping fusion of two proteins. In some embodiments, the two proteins are middle T antigen and an RNA polymerase. In some embodiments, inactivation of the protease may reenable transcription. In some embodiments, the barcode comprises an index comprising greater than or equal to about 6 contiguous base pairs that are specific to the target enzyme. In some embodiments, a single nucleic acid molecule comprising any combination of the first nucleic acid sequence, the second nucleic acid sequence, the third nucleic acid sequence, the fourth nucleic acid sequence, the fifth nucleic acid sequence, the sixth nucleic acid sequence, the seventh nucleic acid sequence, the eighth nucleic acid sequence, and the ninth nucleic acid sequence. In some embodiments, one or more of the first nucleic acid sequence, the second nucleic acid sequence, the third nucleic acid sequence, the fourth nucleic acid sequence, the fifth nucleic acid sequence, the sixth nucleic acid sequence, the seventh nucleic acid sequence, the eighth nucleic acid sequence, and the ninth nucleic acid sequence is a vector comprising a plasmid vector, a viral vector, a cosmid, an artificial chromosome, a region of the host chromosome, or any combination thereof. In some embodiments, the reporter gene encodes: a luciferase enzyme; a fluorescent polypeptide; secreted alkaline phosphatase; ß-galactosidase levansucrase; chloramphenicol acetyltransferase (CAT); antibiotic resistance; or a polymerizing enzyme that, when expressed, binds to a promoter operably linked to a gene encoding a detectable polypeptide to drive expression of the detectable polypeptide. In some embodiments, the expression of the detectable polypeptide is greater than an expression of the detectable polypeptide when the gene encoding the detectable polypeptide is included as the reporter gene. In some embodiments, the system further comprises an exogenous nucleic acid encoding a terpene synthase, a nonribosomal peptide synthetase, or a combination thereof. In some embodiments, the exogenous nucleic acid comprises a second barcode sequence sufficient to identify the terpene synthase. In some embodiments, the second barcode comprises an index, and wherein the index comprises greater than or equal to about 6 nucleotide base pairs that are specific to the terpene synthase. In some embodiments, the exogenous nucleic acid further comprises an enzyme configured to catalyze condensation of (i) isopentenyl diphosphate (IPP), (ii) dimethylallyl diphosphate (DMAPP), or (iii) a combination of the IPP and the DMAPP. In some embodiments, the enzyme comprises a geranylgeranyl diphosphate synthase (GGPPS). In some embodiments, the exogenous nucleic acid further encodes a cytochrome P450 enzyme, a cytochrome P450 reductase enzyme, a cytochrome b5 enzyme, an oxidase enzyme, an acyltransferase enzyme, a glycosyltransferase enzyme, a halogenase, a peroxidase, or any combination thereof.

Aspects disclosed herein provide isolated cells comprising the system for high throughput screening of bioactive molecules that inhibit a target enzyme. comprising: a first nucleic acid sequence encoding a phosphorylated tyrosine binding domain; a second nucleic acid sequence encoding a repressor element; a third nucleic acid sequence encoding a subunit of RNA polymerase; a fourth nucleic acid sequence encoding a tyrosine kinase substrate; a fifth nucleic acid sequence encoding tyrosine kinase; a sixth nucleic acid encoding the target enzyme, wherein the sixth nucleic acid comprises a barcode sufficient to identify the target enzyme; a seventh nucleic acid encoding an operator for the repressor element; an eighth nucleic acid sequence comprising a binding site for the RNA polymerase; and a ninth nucleic acid sequence encoding a reporter gene. In some embodiments, the isolated cell comprises a prokaryotic cell. In some embodiments, the isolated cell is obtained from a unicellular organism. In some embodiments, the isolated cell comprises a bacterial cell, an algae cell, an archaea cell, a protozoa cell, or a fungal cell. In some embodiments, the fungal cell is a yeast cell. In some embodiments, the bacterial cell is an Escherichia coli (E. coli) cell.

Aspects disclosed herein provide methods of identifying a modulator of the target enzyme, the methods comprising: (a) introducing into a plurality of cells the system for high throughput screening of bioactive molecules that inhibit a target enzyme that links the modulation of the target enzyme with expression of the reporter gene; (b) measuring expression of the reporter gene in the plurality of cells; (c) detecting in a subset of the plurality of cells an increased or decreased expression of the reporter gene as compared to a reference expression level, thereby indicating a presence of the modulator of the target enzyme produced by cells within the subset of the plurality of cells; identifying the first barcode in cells within the subset of the plurality of cells, thereby identifying the target enzyme of the modulator produced by the cells; and optionally, isolating the modulator of the target enzyme produced by the cells to identify the modulator. In some embodiments, introducing into the plurality of cells an exogenous nucleic acid sequence encoding a terpene synthase, wherein the exogenous nucleic acid sequence comprises a second barcode. In some embodiments, the second barcode comprises an index, and wherein the index comprises greater than or equal to about 6 contiguous base pairs that are specific to the terpene synthase. In some embodiments, the exogenous nucleic acid sequence further encodes an enzyme configured to catalyze condensation of (i) isopentenyl diphosphate (IPP), (ii) dimethylallyl diphosphate (DMAPP), or (iii) a combination of the IPP and the DMAPP. In some embodiments, herein the enzyme comprises a geranylgeranyl diphosphate synthase (GGPPS). In some embodiments, the exogenous nucleic acid sequence further encodes a cytochrome P450 enzyme, a cytochrome P450 reductase enzyme, a cytochrome b5 enzyme, an oxidase enzyme, an acyltransferase enzyme, a glycosyltransferase enzyme, a halogenase, a peroxidase, or any combination thereof. In some embodiments, introducing into the plurality of cells another exogenous nucleic acid sequence encoding a metabolic pathway for (i) the IPP, (ii) the DMAPP, (iii) a combination of the IPP and DMAPP, or (iv) molecules resulting from the condensation of the IPP, the DMAPP, or the combination of the IPP or the DMAPP. In some embodiments, identifying the second barcode in the cells within the subset of the plurality of cells, thereby identifying which of the unique exogenous terpene synthase in each of the cells produces the modulator of the target enzyme in that cell. In some embodiments, the measuring is performed by multiplex sequencing genetic information of the cells. In some embodiments, the multiplex sequencing comprises sequencing-by-synthesis, sequencing by transient binding, single-molecule real-time sequencing, ion semiconductor sequencing (Iron Torrent®), pyrosequencing, combinatorial probe anchor synthesis (cPAS), sequencing-by-ligation, nanopore sequencing, or semiconductor-based electronic sequencing (GenapSys™). In some embodiments, the methods may include associating the expression of the reporter gene in each cell of the subset of the plurality of cells with the barcode for each cell using a computer processor programmed to demultiplex genetic information that was sequenced. In some embodiments, associating the expression of the reporter gene in each cell of the subset of the plurality of cells with the second barcode for each cell of the plurality of cells using a computer processor programmed to demultiplex genetic information that was sequenced. In some embodiments, the plurality of cells comprises 10-10¹⁰ colony-forming cells for a single implementation of the method. In some embodiments, culturing the plurality of cells in a growth cell medium, wherein the growth cell medium comprises (i) glycerol at a concentration between about 1% and about 2%, (ii) mevalonate at a concentration comprising less than or equal to about 20 mM, (iii) or a combination of (i) and (ii). In some embodiments, of the modulator of the target enzyme is a modulator of the target enzyme when the expression of the reporter gene detected in (c) is increased. In some embodiments, the modulator of the target enzyme is an activator of the target enzyme when the expression of the reporter gene detected in (c) is decreased.

Aspects disclosed herein provide systems for high throughput screening of bioactive molecules that modulate a target enzyme, the systems comprising: a first nucleic acid sequence encoding a phosphorylated tyrosine binding domain; a second nucleic acid sequence encoding a repressor element; a third nucleic acid sequence encoding a subunit of RNA polymerase; a fourth nucleic acid sequence encoding a tyrosine phosphatase substrate; a fifth nucleic acid sequence encoding tyrosine kinase; a sixth nucleic acid encoding the target enzyme; a seventh nucleic acid encoding an operator for the repressor element; an eighth nucleic acid sequence comprising a binding site for the RNA polymerase; and a ninth nucleic acid sequence encoding a polymerizing enzyme, that when expressed, drives expression of a detectable polypeptide. In some embodiments, the phosphorylated tyrosine binding domain comprises a Src homology 2 (SH2) domain. In some embodiments, the repressor element comprises a cI repressor. In some embodiments, the subunit of the RNA polymerase comprises an omega subunit of the RNA polymerase. In some embodiments, the tyrosine kinase comprises Src kinase. In some embodiments, the target enzyme is a tyrosine phosphatase, a protease, or a combination thereof. In some embodiments, the protease is a viral protease. In some embodiments, the sixth nucleic acid sequence comprises a barcode sufficient to identify the target enzyme. In some embodiments, the barcode comprises an index comprising greater than or equal to about 6 contiguous base pairs that are specific to the target enzyme. In some embodiments, the system further comprises an exogenous nucleic acid encoding a terpene synthase, a nonribosomal peptide synthetase, or a combination thereof. In some embodiments, a single nucleic acid molecule comprising any combination of the first nucleic acid sequence, the second nucleic acid sequence, the third nucleic acid sequence, the fourth nucleic acid sequence, the fifth nucleic acid sequence, the sixth nucleic acid sequence, the seventh nucleic acid sequence, the eighth nucleic acid sequence, and the ninth nucleic acid sequence. In some embodiments, one or more of the first nucleic acid sequence, the second nucleic acid sequence, the third nucleic acid sequence, the fourth nucleic acid sequence, the fifth nucleic acid sequence, the sixth nucleic acid sequence, the seventh nucleic acid sequence, the eighth nucleic acid sequence, and the ninth nucleic acid sequence is vectors comprising a plasmid vector, a viral vector, a cosmid, an artificial chromosome, a region of the host chromosome, or a combination thereof. In some embodiments, the detectable polypeptide comprises a fluorescent polypeptide. In some embodiments, the expression of the detectable polypeptide is greater than an expression of the detectable polypeptide when the gene encoding the detectable polypeptide is included in place of the gene for the polymerizing enzyme. In some embodiments, the RNA polymerase is different than the polymerizing enzyme. In some embodiments, the polymerizing enzyme comprises an RNA polymerizing enzyme. In some embodiments, the RNA polymerizing enzyme comprises T7 RNA polymerase.

Aspects disclosed herein provide isolated cells comprising the system for high throughput screening of bioactive molecules that modulate a target enzyme, the systems comprising: a first nucleic acid sequence encoding a phosphorylated tyrosine binding domain; a second nucleic acid sequence encoding a repressor element; a third nucleic acid sequence encoding a subunit of RNA polymerase; a fourth nucleic acid sequence encoding a tyrosine phosphatase substrate; a fifth nucleic acid sequence encoding tyrosine kinase; a sixth nucleic acid encoding the target enzyme; a seventh nucleic acid encoding an operator for the repressor element; an eighth nucleic acid sequence comprising a binding site for the RNA polymerase; and a ninth nucleic acid sequence encoding a polymerizing enzyme, that when expressed, drives expression of a detectable polypeptide. In some embodiments, the isolated cell comprises a prokaryotic cell. In some embodiments, the isolated cell is obtained from a unicellular organism. In some embodiments, the isolated cell comprises a bacterial cell, an algae cell, an archaea cell, a protozoa cell, or a fungal cell. In some embodiments, the fungal cell is a yeast cell. In some embodiments, the bacterial cell is an Escherichia coli (E. coli) cell.

Aspects disclosed herein provide methods of amplifying expression of a reporter in vivo that is linked to modulation of a target enzyme, the methods comprising: introducing into a cell the system of high throughput screening of bioactive molecules that modulate a target enzyme; and measuring expression of the detectable polypeptide in the cell, wherein the expression of the detectable polypeptide is greater than the expression of the detectable polypeptide when the gene expressing the detectable polypeptide is included in place of the gene for the polymerizing enzyme. In some embodiments, the expression of the detectable polypeptide is greater by at least 2-fold, 3-fold, 4-fold, 5-fold, 10-fold, or 100-fold. In some embodiments, the expression of the detectable polypeptide is greater by between about 2-fold and 100-fold, 3-fold and 90-fold, 4-fold and 80-fold, 5-fold and 70-fold, 6-fold and 60-fold, 7-fold and 50-fold, 8-fold and 40-fold, 9-fold and 30-fold, 10-fold and 20-fold. In some embodiments, the cell comprises a prokaryotic cell. In some embodiments, the cell is obtained from a unicellular organism. In some embodiments, the cell comprises a bacterial cell, an algae cell, an archaea cell, a protozoa cell, or a fungal cell. In some embodiments, the fungal cell is a yeast cell. In some embodiments, the bacterial cell is an Escherichia coli (E. coli) cell.

Aspects disclosed herein provide terpene synthases comprising: an amino acid sequence encoding a terpene synthase, wherein the amino acid sequence comprises a mutation that increases modulation of a target tyrosine phosphatase as compared with an otherwise identical terpene synthase without the mutation. In some embodiments, the terpene synthase comprises γ-humulene synthase (GHS), amorphadiene (AD) synthase, or α-bisabolene (AB) synthase. In some embodiments, the target tyrosine phosphatase comprises a cysteine-specific protein tyrosine phosphatase. In some embodiments, the cysteine-specific protein tyrosine phosphatase comprises a dual-specificity phosphatase (DUSP). In some embodiments, the target tyrosine phosphatase comprises Protein Tyrosine Phosphatase 1B (PTP1B), Protein tyrosine phosphatase non-receptor type 2 (TC-PTP), Protein tyrosine phosphatase non-receptor type 6 (SHP1), Protein tyrosine phosphatase non-receptor type 11 (SHP1), Protein tyrosine phosphatase non-receptor type 12 (PTP-PEST), or Protein tyrosine phosphatase non-receptor type 22 (LYP). In some embodiments, the mutation is a single amino acid mutation. In some embodiments, the amino acid sequence comprises SEQ ID NO: 7, and wherein the mutation comprises A319Q or Y415C, or a combination thereof. In some embodiments, the mutation is with reference to SEQ ID NO: 7, and wherein the mutation comprises (a) A319Q and Y415F, (b) A319Q and S484G, or (c) A319Q and S484G, or a combination thereof. In some embodiments, the mutation comprises an amino acid mutation of an amino acid lacking a hydroxyl group. In some embodiments, the terpene synthase is isolated. In some embodiments, the terpene synthase is purified.

Aspects disclosed herein provide methods of identifying a modulator of the target tyrosine phosphatase, the methods comprising: expressing in a cell the terpene synthase; introducing into the cell an expression system that links the modulation of the target tyrosine phosphatase with expression of a reporter gene; and measuring expression of the reporter gene in the presence of the terpene synthase, wherein an increased expression of the reporter gene as compared with a reference expression level indicates a presence of the modulator of the target tyrosine phosphatase produced by the cell. In some embodiments, the modulator of the target tyrosine phosphatase comprises himachalol, α-himachalene, or β-himachalene. In some embodiments, the cell comprises a prokaryotic cell. In some embodiments, the cell is obtained from a unicellular organism. In some embodiments, the cell comprises a bacterial cell, an algae cell, an archaea cell, a protozoa cell, or a fungal cell. In some embodiments, the fungal cell is a yeast cell. In some embodiments, the bacterial cell is an Escherichia coli (E. coli) cell. In some embodiments, culturing the cell in a growth cell medium, wherein the growth cell medium comprises glycerol at a concentration of less than or equal to about 2% (by volume). In some embodiments, culturing the cell in a growth cell medium, wherein the growth cell medium comprises mevalonate at a concentration of less than or equal to about 20 mM. In some embodiments, the expression system comprises: a first nucleic acid sequence encoding a phosphorylated tyrosine binding domain, wherein the phosphorylated tyrosine binding domain optionally comprises a Src homology 2 (SH2) domain; a second nucleic acid sequence encoding a repressor element, wherein the repressor element is optionally a cI repressor; a third nucleic acid sequence encoding a subunit of an RNA polymerase, wherein the subunit of the RNA polymerase comprises an omega subunit of the RNA polymerase (RpoZ); a fourth nucleic acid sequence encoding a tyrosine phosphatase substrate; a fifth nucleic acid sequence encoding a tyrosine kinase, wherein optionally the tyrosine kinase optionally comprises Src kinase; a sixth nucleic acid encoding the target tyrosine phosphatase; a seventh nucleic acid encoding an operator for the repressor element, wherein the repressor element optionally comprises a cI repressor; an eighth nucleic acid sequence comprising a binding site for the RNA polymerase; and a ninth nucleic acid sequence encoding the reporter gene. In some embodiments, the expression system further comprises a tenth nucleic acid sequence encoding Hsp90 co-chaperone Cdc37. In some embodiments, the first nucleic acid sequence and the second nucleic acid sequence encode the phosphorylated tyrosine binding domain fused with the repressor element. In some embodiments, third nucleic acid sequence and the fourth nucleic acid sequence encode the subunit of the RNA polymerase fused with the tyrosine phosphatase substrate. In some embodiments, the first nucleic acid sequence and the fourth nucleic acid sequence encode the phosphorylated tyrosine binding domain fused with the tyrosine phosphatase substrate. In some embodiments, the second nucleic acid sequence and the third nucleic acid sequence encode the repressor element fused with the subunit of the RNA polymerase. In some embodiments, the tyrosine phosphatase substrate comprises a polypeptide, wherein the polypeptide comprises a tyrosine residue configured to: be phosphorylated by the Src kinase; dephosphorylated by the target tyrosine phosphatase; bind to the SH2 domain when the tyrosine residue is phosphorylated; bind to the SH2 domain with less binding affinity when the tyrosine residue is dephosphorylated as compared to the binding affinity between the tyrosine residue and the SH2 domain when the tyrosine residue is phosphorylated; or any combination of (i) to (iv). In some embodiments, the tyrosine phosphatase substrate comprises a substrate domain derived from a hamster polyomavirus middle T antigen (MidT). In some embodiments, the expression system further comprises a first barcode sequence operably linked to the sixth nucleic acid sequence, wherein the first barcode is sufficient to identify the target tyrosine phosphatase. In some embodiments, the barcode comprises an index, and wherein the index comprises greater than or equal to about 6 contiguous base pairs that are specific to the target tyrosine phosphatase. In some embodiments, the expression system further comprises a single nucleic acid molecule comprising any combination of the first nucleic acid sequence, the second nucleic acid sequence, the third nucleic acid sequence, the fourth nucleic acid sequence, the fifth nucleic acid sequence, the sixth nucleic acid sequence, the seventh nucleic acid sequence, the eighth nucleic acid sequence, and the ninth nucleic acid sequence. In some embodiments, the single nucleic acid molecule comprises a vector comprising a plasmid vector, a viral vector, a cosmid, an artificial chromosome, or a region of a chromosome of the cell. In some embodiments, one or more of the first nucleic acid sequence, the second nucleic acid sequence, the third nucleic acid sequence, the fourth nucleic acid sequence, the fifth nucleic acid sequence, the sixth nucleic acid sequence, the seventh nucleic acid sequence, the eighth nucleic acid sequence, and the ninth nucleic acid sequence is a vector comprising a plasmid vector, a viral vector, a cosmid, an artificial chromosome, a region of the host chromosome, or any combination thereof. In some embodiments, the reporter gene encodes: a luciferase enzyme; a fluorescent polypeptide; secreted alkaline phosphatase; ß-galactosidase levansucrase; chloramphenicol acetyltransferase (CAT); antibiotic resistance; or a polymerizing enzyme that, when expressed, binds to a promoter operably linked to a gene encoding a detectable polypeptide to drive expression of the detectable polypeptide. In some embodiments, the expression of the detectable polypeptide is greater than an expression of the detectable polypeptide when a gene encoding the detectable polypeptide is included as the reporter gene. In some embodiments, the expressing the terpene synthase comprises introducing an exogenous nucleic acid into the cell, wherein the exogenous nucleic acid encodes the terpene synthase. In some embodiments, the exogenous nucleic acid comprises a second barcode sequence sufficient to identify the terpene synthase. In some embodiments, the second barcode comprises an index, and wherein the index comprises greater than or equal to about 6 contiguous base pairs that are specific to the terpene synthase. In some embodiments, the exogenous nucleic acid further encodes an enzyme configured to catalyze condensation of isopentenyl diphosphate (IPP), dimethylallyl diphosphate (DMAPP), or a combination of IPP and DMAPP. In some embodiments, the enzyme comprises a geranylgeranyl diphosphate synthase (GGPPS). In some embodiments, the exogenous nucleic acid further encodes a cytochrome P450 enzyme, a cytochrome P450 reductase enzyme, a cytochrome b5 enzyme, an oxidase enzyme, an acyltransferase enzyme, a glycosyltransferase enzyme, a halogenase, a peroxidase, or any combination thereof. In some embodiments, the expressing the terpene synthase further comprises introducing another exogenous nucleic acid encoding a metabolic pathway for (i) the IPP, (ii) the DMAPP, (iii) molecules resulting from the condensation of the IPP or the DMAPP, or the combination of IPP and DMAPP, or (iv) any combination thereof. In some embodiments, isolating the modulator of the target tyrosine phosphatase. In some embodiments, the reference expression level is derived from a reference cell expressing the second nucleic acid sequence that is modified such that the tyrosine phosphatase substrate contains a mutation that inhibits its binding to the SH2 domain. In some embodiments, the reference expression level is derived from a reference cell comprising a modified terpene synthase comprising a mutation that reduces its activity as compared with an otherwise identical terpene synthase that does not have the mutation.

Aspects provided herein provide isolated nucleic acid molecules encoding the terpene synthase comprising: an amino acid sequence encoding a terpene synthase, wherein the amino acid sequence comprises a mutation that increases modulation of a target tyrosine phosphatase as compared with an otherwise identical terpene synthase without the mutation. In some embodiments, the isolated nucleic acid molecule is a vector comprising a plasmid vector, a viral vector, a cosmid, an artificial chromosome, or a region of a host chromosome.

Aspects disclosed herein provide isolated cells comprising the terpene synthase comprising: an amino acid sequence encoding a terpene synthase, wherein the amino acid sequence comprises a mutation that increases modulation of a target tyrosine phosphatase as compared with an otherwise identical terpene synthase without the mutation. In some embodiments, the cell comprises a prokaryotic cell. In some embodiments, the cell is obtained from a unicellular organism. In some embodiments, the cell comprises a bacterial cell, an algae cell, an archaea cell, a protozoa cell, or a fungal cell. In some embodiments, the fungal cell is a yeast cell. In some embodiments, the bacterial cell is an Escherichia coli (E. coli) cell.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the inventive concepts are set forth with particularity in the appended claims. A better understanding of the features and advantages of the present inventive concepts will be obtained by reference to the following detailed description that sets forth illustrative embodiments, in which the principles of the inventive concepts are utilized, and the accompanying drawings of which:

FIGS. 1A-1D show an experimental framework to evolve terpene synthases according to embodiments of the present disclosure. FIG. 1A shows a promiscuous terpene synthase: γ-humulene synthase (GHS) that binds to farnesyl diphosphate (1) releases the terminal diphosphate and cyclizes the resulting trans- or cis-farnesyl cation into over 50 terpenoid products, a subset of which appear here. Highlights show terpenoids generated from a shared intermediate. FIG. 1B shows a crystal structure of PTP1B bound to amorphadiene, an allosteric inhibitor (AD, pdb entry 6W30). An overlay of a competitive inhibitor (UN7) highlights the active site (aligned pdb entry 2F71). FIG. 1C provides a schematic of a genetically encoded systems for terpenoid biosynthesis (left) and inhibitor detection (right), according to an embodiment herein. FIG. 1D shows a selection scheme for identifying GHS mutants that generate PTP1B inhibitors.

FIGS. 2A-2E. show results from site-saturation mutagenesis of GHS (with reference to SEQ ID NO: 7) according to embodiments of the present disclosure. FIG. 2A shows a homology model for GHS showing residues targeted for site saturation mutagenesis (SSM). A substrate analog (circles) is positioned by aligning the crystal structure of 5-epi-aristolochene synthase (pdb entry 5eat). FIG. 2B shows sesquiterpene production by GHS, GHSA319Q, and GHS415C. FIG. 2C shows the total terpene titers (mg/L, longifolene equivalents) for each strain. FIG. 2D shows the intracellular terpene titers of compounds 2, 8, and 10 (μM, longiolene equivalents). FIG. 2E shows spectinomycin resistance conferred by mutants of GHS. X indicates inactive B2H (e.g., a substrate domain with a Y/F mutation). Error bars in B-D denote standard deviation for n≥3 biological replicates.

FIGS. 3A-3C show a reduction of fitness advantage conferred by farnesyl diphosphate (FPP) according to embodiments of the present disclosure. FIG. 3A shows the terpenoid pathway produces two potential inhibitors of protein tyrosine phosphatase 1B (PTP1B). FIG. 3B shows the initial rates of PTP1B-catalyzed hydrolysis of p-Nitrophenyl Phosphate (PNPP) in the presence of increasing concentrations of FPP ([PTP1B]=50 nM; [pNPP]=5 mM). A linear fit provides a rough estimate of IC50 (inset). FIG. 3C shows spectinomycin resistance conferred by an empty vector (e.g., pTS without a TS gene) and GHS A319Q in different media. X=a B2H system with a Y/F mutation in the peptide substrate. Error bars in B denote standard error for n=3 independent measurements.

FIGS. 4A-4D shows a multi-site mutant analysis according to embodiments of the present disclosure. FIG. 4A shows the antibacterial resistance for multi-site mutants of terpene synthases provided herein. FIG. 4B shows the terpenoid titers of the indicated products for different mutants of GHS. Error bars denote propagated standard deviation for n≥3 biological replicates. FIG. 4C shows a schematic representation of a non-limiting hypothesis that mutations to the Y415 residue (with reference to SEQ ID NO: 7) shift production towards himachalane-type sesquiterpenes. FIG. 4D depicts a schematic representation of the mechanism for the formation of himachalol, ß-himachalene, and γ-humulene.

FIGS. 5A-5C show a non-limiting protease-dependent system for controlling transcription according to embodiments of the present disclosure. FIG. 5A shows a non-limiting example of the general architecture for a protease-inhibited bacterial two-hybrid system. In this figure, components include (i) a phosphotyrosine substrate (e.g., MidT) fused to the omega subunit of RNA polymerase (RpoZ) with a linker containing a protease cleavage site (CS), (ii) a superbinder Src homology 2 domain (e.g., SH2) fused to a DNA-binding protein (cI), (iii) a kinase (cSrc) and a chaperone to aid in kinase folding (e.g., Cell Division Cycle 37, HSP90 Cochaperone (CDC37)), (iv) a protease, (v) an optimized two-hybrid promoter (pLacZopt) driving expression of a gene of interest (GOI), and (vi) binding sites for RNA polymerase (RNAP) and cI (cI op). In this schematic, FIG. 5A shows that Src kinase phosphorylates MidT, enabling binding to SH2 and localization of RNAP to drive transcription of the GOI in the presence of an active protease inhibitor. Also provided are non-limiting examples of the system in FIG. 5A for proteases: HIV-1 protease (HIV-1Pr) and 3-chymotrypsin-like protease (3ClPro) from SARSCOV2 (FIG. 5B shows HIV-1 Protease recognition site with reference to the SEQ ID NO: 26. FIG. 5C shows 3Cl Pro Protease recognition site with reference to the SEQ ID NO: 27. FIG. 5B-5C shows computationally designed ribosomal binding site (RBS)'s, the indicated cleavage sites in the MidT/RpoZ linker, and a spectinomycin resistance gene (aadA, “denoted as SpecR”). “*” indicates an inactive protease (HIV1-PR: D25N mutation, 3ClPro: H41A mutation).

FIGS. 6A-6B show a screening technique of terpenoid pathways for protease inhibitors according to embodiments of the present disclosure. FIG. 6A depicts a schematic that illustrates terpenoid pathways introduced on two plasmids containing (i) the isoprenoid utilization pathway (IUP) precursor pathway to convert isoprenol into FPP or Geranylgeranyl pyrophosphate synthase (GGPP) and (ii) a terpene synthase pathway containing one of 37 genes from an in-house library. These pathway combinations were combined with the HIV1-Pr and 3ClPro B2H systems. FIG. 6B shows the survival of the cells for each of the 37 genes from the in-house library.

FIGS. 7A-7B show the growth of E. coli cells harboring bacterial two-hybrid (B2H) systems for different protein tyrosine phosphatases (PTPs) according to embodiments of the present disclosure. Subscripts indicate the truncation used for each enzyme. Protein tyrosine phosphatase 1B (PTP1B₄₀₅) and Protein Tyrosine Phosphatase Non-Receptor Type 2 (TCPTP)₃₈₇ include C-terminal regions that extend beyond the conserved catalytic PTP domain. All mutations in parentheses are inactivating except for PEST (E57D), which is associated with cancer. FIG. 7A shows cells harboring functional B2H systems for PTP1B₃₂₁, PTP1B₄₀₅, TCPTP₃₁₇, TCPTP₂₈₇, and PEST_{E57D}; active PTPs reduce antibiotic resistance, and PTP inactivation enhances resistance. FIG. 7B shows non-functional B2H systems for STEP and SHP2; active PTPs do not reduce antibiotic resistance under the conditions tested. Toggling—and, in particular, enhancing—active enzyme expression provides a logical step for making these B2H systems functional.

FIGS. 8A-8B show a high-throughput screening approach according to embodiments of the present disclosure. FIG. 8A shows plasmid(s) containing (i) a PTP B2H for different PTPs, (ii) the IUP precursor pathway accompanied by genes that enable the conversion of isoprenol into geranyl pyrophosphate (GPP), farnesyl pyrophosphate (FPP), and/or Geranylgeranyl pyrophosphate synthase (GGPP), and (iii) a terpene synthase pathway containing one of 37 genes from an in-house library, where each gene of the 37 genes is barcoded with a unique barcode sequence (“BC”). FIG. 8B shows how the barcoded terpene synthase pathways are transfected into cells, and cells that produce a signal indicative of PTP modulation are selected and pooled, and the barcoded regions of DNA isolated from cells grown in the presence of different concentrations of antibiotic is amplified with a secondary barcode that marks the screening conditions and PTP, and then multiplex sequencing is performed to identify terpene synthase pathways that produce modulators for each PTP.

FIGS. 9A-9B show a fluorescent B2H yield from an amplification with T7 RNA Polymerase (RNAP) according to an embodiment of the present disclosure. FIG. 9A shows a schematic of phosphorylation dependent B2H system, where the gene of interest (GOI) encodes a RNA polymerizing enzyme (e.g., T7RNAP), that when expressed, induces expression of a detectable polypeptide, such as a fluorescent protein (FP). FIG. 9B shows that the signal observed when expressed in a cell is amplified by over 4-fold using this strategy, as compared to an otherwise comparable phosphorylation dependent B2H system with a GOI that encodes the FP itself.

FIGS. 10A-10D shows product profiles of mutants identified a single-site mutant analysis according to embodiments of the present disclosure (E. coli s1030+pTS+pMBIS+pB2H in 10-ml TB media). FIG. 10A provides chromatograms that show extracted ions (m/z=204) scaled to injection size, measured by peak area of an internal standard (20 μg/mL methyl abietate, m/z=316). FIG. 10B shows the titers of the dominant products of gamma-humulene synthase mutants A319Q and Y415C with reference to SEQ ID NO: 7. Compound numbering refers to the compounds depicted in FIG. 1A and FIG. 10C. FIG. 10C shows the structure of a protease inhibitor, α-bisabolol, identified from a screen carried out with B2H systems. FIG. 10D shows the spectinomycin resistance conferred by mutants of GHS. Images show the growth of E. coli harboring pMBIS, pTS, and pB2H on agar plates seeded from drops of liquid culture (D/A=D343A, a mutation that inactivates GHS).

FIGS. 11A-11C show defining gene deletions as (i) all or part of the terpene synthase missing in a Sanger sequencing result or (ii) no band, a band of incorrect size, or multiple bands in a colony PCR, the frequency of incomplete genes was quantified in (FIG. 11A) the site saturation mutagenesis screen using GHS WT as a template, (FIG. 11B) the error-prone PCR screen using GHS A319Q as a template, and (FIG. 11C) the site saturation mutagenesis screen using A319Q as a template. Labels in all charts indicate counts of full or incomplete gene.

FIGS. 12A-12B show an analysis of antibiotic resistance conferred by an empty vector according to embodiments of the present disclosure. The spectinomycin resistance can be conferred by an empty vector (e.g., pTS without a terpene synthase (TS) gene) and A319Q (with reference to SEQ ID NO: 7) in different media. Images show the growth of E. coli harboring pMBIS, pTS, and pB2H on agar plates seeded from drops of liquid culture (X=a B2H system with a Y/F mutation in the substrate). These biological replicates, which were carried out on different days, were seeded at low (OD₆₀₀=0.1; FIG. 12A) and high (OD₆₀₀=0.5; FIG. 12B) optical densities. Media compositions previously shown to increase intracellular FPP concentrations (left to right) reduce the fitness advantage of the empty vector but not GHSA319Q.

FIGS. 13A-13B show an analysis of mutants of GHS according to embodiments of the present disclosure. FIG. 13A shows the product profiles of mutants that were identified in screens of SSM and ePCR libraries that used GHSA319Q as a parent template. The chromatograms show extracted ions (m/z=204) scaled to injection size, which were determined from the peak area of an internal standard (20 μg/mL methyl abietate, m/z=50-500). FIG. 13B shows the spectinomycin resistance conferred by mutants of GHS that caused major shifts in product profile (relative to A319Q). Images show the growth of E. coli harboring pMBIS, pTS, and pB2H on agar plates seeded from drops of liquid culture.

FIGS. 14A-14B show an analysis of terpenoids produced by multi-site mutants according to embodiments of the present disclosure. FIG. 14A shows the product profiles of mutants of GHSA319Q identified in screens of site saturation mutagenesis (SSM) libraries. The chromatograms show extracted ions (m/z=204) scaled such that the height of the largest peak=1. Mutations S484A and S484G enable the production of products that do not have high confidence matches (R-match>900) in the NIST Mass Spectral Library (22-24). Products 23 and 24 are not produced by any other GHS variants examined in this study. FIG. 14B shows the himachalane fraction (e.g., the fraction of total terpenoids comprising α-, β-, and γ-himachalene and himachalol) for several mutants of GHS. Mutations to residue Y415 shown in FIG. 14B enhance the production of himachalanes. Error bars in FIG. 14B denote propagated standard error for n>3 biological replicates. * indicates p<0.05. Table 18 provides details on hypothesis testing.

FIG. 15 shows a standard curve for p-nitrophenol (pNP) according to embodiments of the present disclosure.

FIGS. 16A-16B show an analysis of substrate cleavage in the B2H system according to embodiments of the present disclosure with reference to SEQ ID NO: 26, SEQ ID NO: 30, SEQ ID NO: 31, SEQ ID NO: 32, SEQ ID NO: 33, and SEQ ID NO: 27. FIG. 16A shows performance of the protease cleavage sites (“CS”) for various linkers tested in the B2H system that contains the two depicted plasmid. The upper plasmid encoded RpoZ-CS-MidT, a SH2-cI, cSrc, CDC37, and an optimized two-hybrid promoter (LuxAB) driving expression of a GOI; and the lower plasmid containing a protease under control of a constitutive promoter (pBAD), see for e.g., FIG. 5A, for another schematic depicting the protease B2H system in further detail. FIG. 16B shows a schematic representation of the CS for SARS-COV/3CLpro proteases and RpoZ with reference to SEQ ID NO: 29 and a SARS-COV 3CLpro cleavage site XXXLQX where X1 and X3 can be any amino acid, X2 can be A/S/T, and X6 can be A/S.

FIG. 17 shows terpene production by E. coli cells harboring plasmids that encode a bacterial two-hybrid system (pB2H), an isopentenol utilization pathway (pIUP) and a prenyltransferase necessary for producing relevant terpenoids, and a terpene synthase (pTS) according to embodiments of the present disclosure. FIG. 17A shows the production of sesquiterpene (amorphadiene) and a diterpene (abietadiene) assessed via hexane extract from a liquid culture (e.g., liquid media and cells). FIG. 17B shows estimates of the intracellular production of these compounds assessed via extract from the cell pellet (cells only).

FIG. 18 shows a cladogram of terpene synthase genes that could be used in the systems and methods disclosed herein according to embodiments of the present disclosure.

FIG. 19 shows the effect of a peptide insertion in the linker between the kinase substrate (e.g., MidT) and polymerase subunit (e.g., RP) on performance of B2H system linking PTP1B inactivation (C215S) (with reference to SEQ ID NO: 6) to a luminescent output (LuxAB expression with reference to SEQ ID NO: 34) as compared to wild type. In this figure, the following insertions were tested: HIV-1 insertion: KARVL*AEAM (SEQ ID NO: 35); 3CLsubs insertion: AVLQ*SGFR (SEQ ID NO: 36); and Uniq: 75 amino acid peptide (LRGG*) (SEQ ID NO: 37). In this figure, the “*” indicates a protease cleave site within the protease recognition motif.

FIG. 20 shows a screen of protease activity on different protease recognition motifs contained within the B2H system according to embodiments of the present disclosure.

FIG. 21 shows the tailoring of HIV-1 protease (HIV-1pr) expression in B2H systems with native phosphatase RBS sequences and without protease-substrate insertions according to embodiments of the present disclosure.

FIG. 22 shows the tailoring of HIV-1pr expression in B2H systems with engineered RBS sequences of target TIRs with and without HIV-1pr recognition motif insertions according to embodiments of the present disclosure.

FIG. 23 shows a performance of HIV-1pr-expressing B2H systems with various protease-recognition motifs according to embodiments of the present disclosure.

FIGS. 24A-24B show a B2H system according to embodiments present in this disclosure. FIG. 24A is a schematic of an embodiment of a B2H system presented herein. FIG. 24B shows performance of a B2H system under different conditions including temperature, incubation time, and concentration of antibiotic (e.g., spectinomycin) according to embodiments of the present disclosure.

FIG. 25 shows B2H system performance under different conditions including pH and concentration of antibiotic (e.g., spectinomycin) according to embodiments of the present disclosure.

FIGS. 26A-26B show an RBS library selection for development of B2H systems containing SARS-COV-2 papain-like protease (PLpro) according to embodiments of the present disclosure. FIG. 26A shows antibacterial resistance of the PLpro constructs provided in FIG. 26B.

FIG. 26B describes the RBS sequences including the Degenerate RBS in reference to SEQ ID NO: 38, and alternative RBS sequences in references to SEQ ID NO: 39, SEQ ID NO: 40, SEQ ID NO: 41, and SEQ ID NO: 42.

FIGS. 27A-27C show a rational recombination of mutants of GHS that enhance antibiotic resistance in a growth-coupled assay according to the embodiments of the present disclosure. FIG. 27A shows an extracted ion chromatogram (m/z=204) for GHSA319Q/Y415C+pB2H and pMBIS in s1030 cells. FIG. 27 B shows titers of total terpenes and major components of GHSA319Q/Y415C, GHSA319Q, GHSY415C. Error bars denote standard deviation for n>3 biological replicates. FIG. 27C show drop-based plating results for single and combined GHS mutants.

FIG. 28 shows an embodiment of a bacterial two-hybrid system that detects protease inhibitors. When a protease inhibitor is absent, protease cleaves the linker at the cleave site (circle) to release the kinase substrate and RPω such that RPω is not recruited to the RPω binding domain and there is no transcription of the gene of interest (e.g., reporter gene) (upper schematic). When a protease inhibitor is present, protease does not cleave the linker and the kinase substrate RPω is recruited to the RPω binding domain to turn on transcription of the gene of interest (bottom schematic).

FIG. 29 shows an analysis of a functional B2H system that detects protease inhibitors and contains a gene for spectinomycin resistance as a gene of interest.

FIGS. 30A-30C show the results of a screen for inhibitors of 3CLpro. FIG. 30A shows an analysis of antibiotic resistance conferred by several pathways in the presence of a B2H system that detects inhibitors of 3CLpro. FIG. 30B shows 3CLpro-mediated hydrolysis of a FRET peptide under different concentrations of α-bisabolol. FIG. 30C plots the percent inhibition of 3CLpro by different concentrations of α-bisabolol. A fit to this data indicates a half-maximal inhibitory concentration (IC₅₀) of around 3 micromolar.

FIG. 31 shows a 1 hour (1H) nuclear magnetic resonance (NMR) spectrum of purified amorphadiene.

FIG. 32 shows an alignment of two crystal structures of PTP1B bound to allosteric inhibitors.

FIG. 33 shows an analysis of 3-(3,5-dibromo-4-hydroxybenzoyl)-2-ethyl-N-[4-[(2-thiazolylamino) sulfonyl]phenyl]-6-benzofuransulfonamide (BBR) binding to PTP1B in the presence and absence of amorphadiene.

FIGS. 34A-34B show an analysis of PTP1B-mediated hydrolysis of p-nitrophenylphosphate (pNPP), a chromogenic substrate, in the presence of different inhibitors. FIG. 34A shows the inhibition of PTP1B by amorphadiene. FIG. 34B shows the inhibition of PTP1B by a derivative of amorphadiene.

FIGS. 35A-35C shows an analysis of a non-ribosomal peptides such as a dipeptide pyrazine. FIG. 35A shows high-performance liquid chromatography (HPLC)-UV for non-ribosomal peptides according to an embodiment herein. FIG. 35B shows the measurement of molecular weight of a non-ribosomal peptide by HPLC mass spectroscopy (MS). FIG. 35C shows the calculated molecular weight of a non-ribosomal peptide, which matches the measured result in FIG. 35B, confirming the molecular weight of the compound, according to an embodiment herein.

FIG. 36 shows an analysis of fluorescence activated cell sorting (FACS) of cells that contain both (i) a bacterial two-hybrid system that links inactivation of PTP1B to the expression of a gene for a T7 RNA polymerase and (ii) a system in which the T7 RNA polymerase transcribes the gene for a fluorescent protein.

FIG. 37 shows an analysis of optical switches in which a light-sensitive interaction between (i) a variant of a light-oxygen-voltage 2 (LOV2) domain that contains a bacterial SsrA peptide in reference to SEQ ID NO: 44 and (ii) a SspB protein controls transcription of a gene of interest (GOI) in reference to SEQ ID NO: 48.

FIG. 38 shows examples of microbial systems encoded with a human therapeutic objective and biosynthetic pathways to identify metabolic pathways that achieve the human therapeutic objective.

FIGS. 39A-39D show non-limiting examples of a B2H system for detecting inhibitors of therapeutic targets disclosed herein. FIG. 39A shows a B2H system for detecting inhibitors of protein tyrosine phosphatase 1B (PTP1B). FIG. 39B shows a B2H system adapted to detect protease inhibitors. FIG. 39C shows a case when the inhibition of the target protease leaves the RpoZ-MidT fusion intact, enabling transcription of the gene of interest (GOI). FIG. 39D shows a case when, in the absence of inhibitors, the target protease “breaks” (via proteolysis) the RpoZ-MidT fusion, preventing transcription of the GOI.

FIGS. 40A-40C show the development of some B2H systems. FIG. 40A shows converting the B2H from FIG. 39A into the versions from FIGS. 39B-39D by (i) adding a protease recognition (PR) motif to the RpoZ-MidT protein, (ii) inactivating PTP1B, and (iii) adding LuxAB as the GOI. Adding a PR reduces the dynamic range by about two-fold. FIG. 40B shows the use of an arabinose-inducible plasmid to titrate active and inactive protease alongside the B2Hs from FIG. 40A. Active protease reduced luminescence for three PR architectures: HIVpro (0A and 4A linkers) and 3CLpro (only 4A). FIG. 40C shows screening of proteases against different cleavage sites. Controls: X, inactive protease. Error=SE of n≥3 technical replicates.

FIG. 41 shows an example where all biosynthetic pathways are screened against all protease targets with a primary DNA barcode (dark grey) and secondary DNA barcode (light grey). Pathways enriched in the presence of antibiotic generate potential inhibitors of each protease (identified with the second barcode).

FIGS. 42A-42C show data from PTP-based B2H systems that supports methods for high-throughput screens and directed evolution. FIG. 42A shows heatmaps that show the log 2-enrichment of 37 terpenoid pathways screened against the catalytic domains (C) and full-length versions (F) of PTPN1, PTPN2, and PTPN12. FIG. 42B shows drop-based plating of E. coli harboring a PTP1B-based B2H system and variants of γ-humulene synthase generated via SSM (X: inactive B2H). FIG. 42C shows that A319Q/Y415F, which enhances resistance, produces significantly more himachalol than the other two mutants.

FIG. 43 shows some structural variants of α-bisabolol according to some embodiments herein.

FIGS. 44A-44D show performance of bacterial two-hybrid (B2H) system for guiding the discovery and assembly of protease inhibitors. FIG. 44A shows inhibition of a target protease prevents proteolysis of PRI, enabling a protein-protein interaction that activates transcription of a resistance gene (SpecR). FIG. 44B shows a B2H system for 3CL protease. Inactivation of 3CLpro (x) enhances spectinomycin resistance. FIG. 44C shows an inhibitor of 3CL protease identified with the B2H system. FIG. 44D shows an inhibition of 3CL protease by a mixture containing α-bisabolol (SE for n≥3 technical replicates).

FIGS. 45A-45G show performance of a mevalonate-dependent isoprenoid pathway, a terpene synthase, and a B2H system that links the inactivation of PTP1B to the expression of a resistance gene. FIG. 45A is a schematic of the mevalonate-dependent isoprenoid pathway, a terpene synthase, and B2H system. FIG. 45B shows a growth-coupled assay for terpene synthases that improve resistance. FIG. 45C shows terpene synthases with different products. FIG. 45D shows the results of a screen: (B2H*, constitutively active B2H; ABSD404A/D621A, inactive ABS). ADS confers the greatest antibiotic resistance. FIG. 45E show the titer of amorphadiene (AD) in the ADS strain exceeds its IC50 for PTP1B; the titer of Taxadiene in the TXS strain does not. FIG. 45F shows the IC₅₀ for PTP1B for the B2H system shown in FIG. 45A. FIG. 45G shows a crystal structure of PTP1B with a competitive inhibitor (circle) and AD, allosteric hit (square; PDB 6W30). Error=SE of n≥3 technical replicates.

FIGS. 46A-46B is a schematic representation of severe acute respiratory syndrome (SARS) virus binding to its cognate receptor, angiotensin converting enzyme 2 (ACE2), expressed on a cell surface of a host. FIG. 46A show an example virus structure of SARS (e.g., SARS-COV-2). FIG. 46B shows that Transmembrane Serine Protease 2 (TMPRSS2) primes the spike protein for binding to ACE2, which mediates invasion of the cell. Proteases 3clPro and PlPro cleave polyproteins into active, fully folded subunits.

FIGS. 47A-47C shows a B2H system that links protease inhibition to GOI transcription in E. coli. FIG. 47A shows a schematic of the B2H system. FIG. 47A shows that binding of B1 to B2 enables GOI transcription. Proteolysis of a recognition site (PRI) on the B2-RpoZ fusion disrupts transcription; protease inhibition reenables it. FIG. 47B shows the B2 component of a phosphorylation-mediated B1-B2 interaction; proteolysis of the protease recognition site on B2 prevents it from activating transcription by binding to B1. FIG. 47C shows the effects of adding 0-4 amino acids on either side of each recognition site (PRI in FIG. 47A and FIG. 47B). FIG. 47C shows 3CLpro in reference to SEQ ID NO: 36, SEQ ID NO: 49, and SEQ ID NO: 50; HIVpro in reference to SEQ ID NO: 35; PLpro in reference to SEQ ID NO: 37; DENVpro/WNVpro in reference to SEQ ID NO: 52, and USP7 in reference to SEQ ID NO: 25. In this system, Src kinase phosphorylates a substrate domain, causing it to bind to a Src homology 2 (SH2) domain, and the substrate-SH2 complex activates transcription of the GOI. PTP1B dephosphorylates the substrate domain, preventing transcription; the inactivation of PTP1B reenables it.

FIGS. 48A-48C shows the development of some B2H systems. FIG. 48A shows converting the B2H system from FIG. 39A by (i) adding a protease recognition (PR) site to the RpoZ-substrate linker, (ii) inactivating PTP1B, and (iii) adding LuxAB as the GOI. Adding a PR reduced dynamic range by about 2×. FIG. 48B shows using an arabinose-inducible plasmid to titrate active and inactive protease alongside the B2H systems from FIG. 48A. Active protease reduced luminescence for three PR architectures: HIVpro (0A and 4A linkers) and 3CLpro (only 4A). FIG. 48C shows screened proteases against different cleavage sites. Controls: X, inactive protease. Error=SE of n≥3 technical replicates.

FIGS. 49A-49B show examples of spectinomycin-based B2H systems. FIG. 49A shows complete B2H systems for HIVpro, 3CLpro, and PTP1B (for comparison). FIG. 49B shows a screen of RBSs for the PLpro system yielded several “hits” that confer sensitivity to spectinomycin. Substrates: LRGG (PLpro substrate) and Ubiquitin, another substrate of PLpro.

FIG. 50 shows a structure of the Dengue virus protease. The NS3 protease can adopt open (inactive) and closed (active) states. NS2B stabilizes the closed state and becomes part of the active site (PDB 4M9M).

FIGS. 51A-51B shows various terpene synthase genes of the clades tested. FIG. 51A shows a cladogram of terpene synthase genes. A B2H screen of 24 uncharacterized genes from 6 characterized and 2 uncharacterized clades uncovered A0A0C9VSL7, shown in FIG. 51B, which produces (+)-1 (10),4-cadinadiene as a dominant product. FIG. 51C shows an estimated IC50. Error 95 CI for n≥3.

FIG. 52 shows some products of terpene synthases, according to some embodiments herein.

FIG. 53 shows an example of a pyrazine dipeptide generated by GupB (a 3-module enzyme) and Sfp in E. Coli, according to some embodiments herein.

FIG. 54 shows examples of phenylpropanoid biosynthesis. Modular pathways are assembled that facilitate combinatorial biosynthesis. The HPLC chromatogram depicts a culture extract from an E. coli strain harboring flavin-dependent hologenase, rdc2, grown in the presence of exogenously added resveratrol.

FIGS. 55A-55C show an example of an analytical workflow for an amorphadiene-producing strain of E. coli. FIG. 55A shows GC-MS chromatograms for extracts from solid and liquid media. Amorphadiene (AD) is the major peak in both. FIG. 55B shows a TLC plate for two fractions from silica chromatography. AD is at the top right corner of the plate. FIG. 55C shows a 1H-NMR for a crude extract and purified AD.

FIG. 56 shows kinetic data for eucalyptol, suggesting that it is not an inhibitor.

FIG. 57 shows a crystal of 3CLpro (2.1 Å).

FIGS. 58A-58C show the development of some B2H systems. FIG. 58A shows converting a B2H system by (i) adding a protease recognition (PR) site to the RpoZ-substrate linker, (ii) inactivating PTP1B, and (iii) adding LuxAB as the GOI. Adding a PR reduced dynamic range by about 2×. FIG. 58B shows using an arabinose-inducible plasmid to titrate active and inactive protease alongside the B2H systems from FIG. 58A. Active protease reduced luminescence for three PR architectures: HIVpro (0A and 4A linkers) and 3CLpro (only 4A). FIG. 58C shows screened proteases against different cleavage sites and assess their dynamic range (e.g., the ratio of luminescence between 0 and 0.02% arabinose (w/%) as depicted in FIG. 58B. Controls: X, inactive protease. Error=SE of n≥3 technical replicates.

FIG. 59 shows some spectinomycin-based B2H systems for PTP1B, HIVpro, 3CLpro, HIVpro, USP7, and Plpro. X denotes mutations that inactivate the enzyme.

FIG. 60 shows a screen of terpenoid pathways against protease-specific B2H systems from FIG. 59. The B2H systems link spectinomycin resistance to protease inhibition. A diverse library of pathways were assembled by combining distinct modules. In brief, the isopentenol utilization pathway (IUP) was coupled with (i) farnesyl pyrophosphate synthase [FPPS] and (ii) the indicated terpene synthase. For 3CLpro, the three pathways that conferred the greatest survival advantage generated α-bisabolol, β-bisabolene, or eucalyptol as major products.

FIG. 61 shows an analysis of the influence of inhibitors on the melting temperature of PTP1B.

FIGS. 62A-62E show an example of an evolutionary trajectory of a PTP1B inhibitor-synthesizing mutant. FIG. 62A shows the spectinomycin resistance conferred by mutants of GHS. Images show the growth of E. coli strains harboring pMBIS, pTS, and pB2H on agar plates seeded from drops of liquid culture (X denotes a B2H system with a Y/F mutation in the peptide substrate). A319Q/Y415F confers a fitness advantage over A319Q. FIG. 62B shows growth curves for E. coli strains overexpressing variants of GHS (note: pMBIS and pB2H are absent from these strains). Specific growth rates are shown in the plot inset. All mutants enhance specific growth rate, an indication of reduced enzyme toxicity. FIG. 62C shows titers of the three major products of A319Q/Y415F for different variants of GHS. FIG. 62D shows initial rates of PTP1B-catalyzed hydrolysis of pNPP in the presence of increasing concentrations of himachalol. Lines show the best-fit kinetic model of inhibition (Table 17). FIG. 62E shows intracellular titers of the major products from FIG. 62C in three variants of GHS. Error bars in FIG. 62B denote standard error for n>3 biological replicates, error bars in FIG. 62C and FIG. 62E denote standard deviation for n>3 biological replicates, and error bars in FIG. 62D denote standard deviation for n>6 technical replicates.

FIG. 63 shows that in some cases, mutations to Y415 shift production towards himachalanes. The screens uncovered several Y415 mutants that bias production towards himachalanes (primarily β-himachalene and himachalol). Solid lines denote mutants found through biological selection, and dashed lines denote rationally designed mutants.

FIGS. 64A-64B shows several GHSY415 mutants (black arrows) that produce large amounts of himachalanes. FIG. 64A shows Himachalol appears in grey; α-, β-, and γ-himachalene, in dark grey; and other components of the mutants in light grey. Light grey lines denote rationally designed mutants. The inset shows the same distributions scaled to total titer. Error bars denote the standard deviation of n≥3 biological replicates. Representative chromatograms appear in FIG. 70. FIG. 64B shows a reaction scheme for forming himachalane- or humulane-type sesquiterpenoids from a common precursor, according to some embodiments herein.

FIGS. 65A-65B show a GHS and variants of GHS. FIG. 65A shows a homology model of GHS (gray) shows six sites targeted for site saturation mutagenesis (SSM, circle) and 12 additional sites (squares). A substrate analogue (dashed rectangle) is positioned by aligning the crystal structure of 5-epi-aristolochene synthase (RCSB Protein Data Bank (pdb) entry “5EAT”). To identify the highlighted sites, the X-ray crystal structures of α-bisabolene synthase (ABS) and taxadiene synthase (TXS) are aligned, selecting all residues within 8 Å of the substrate analog of the class I active site of TXS, and identifying sites that differ between ABS and TXS (18 in total). FIG. 65B shows a multiple sequence alignment of EIS (CYC1_STRCO) in reference to SEQ ID NO: 55, DSS (TPSD4_ABIGR) in reference to SEQ ID NO: 56, GHS (TPSD5_ABIGR) in reference to SEQ ID NO: 57, ABS (TPSDV_ABIGR) in reference to SEQ ID NO: 58, and TXS (TASY_TAXBR) in reference to SEQ ID NO: 13. Highlights: The six highest-scoring sites selected for SSM (circles) and 12 additional sites (squares). FIG. 75 provides the scores for all 18 sites.

FIGS. 66A-66E show product profiles of mutants identified in a screen of a single-site library. FIG. 66A provide chromatograms for mutant terpene synthases compared to wild type terpene synthase that show an extracted ion (m/z=204) scaled to injection size, which was determined from the peak area of an internal standard (20 μg/mL methyl abietate, m/z=316). FIG. 66B shows chromatograms for mutants showing large shifts in product profile compared to the wild-type enzyme. Chromatograms show an extracted ion (m/z=204) scaled such that the largest peak height=1. FIG. 66C shows titers of dominant products. Compound numbering refers to the scheme in FIG. 1A. FIG. 66D shows the chemical structure of compound 21, α-bisabolol. FIG. 66E shows the spectinomycin resistance conferred by mutants of GHS. Images show the growth of E. coli harboring pMBIS, pTS, and pB2H on agar plates seeded from drops of liquid culture (D/A=D343A, a mutation that inactivates GHS). Error bars in C denote standard deviation of n≥3 biological replicates.

FIGS. 67A-67C show performance of Y415C and double mutant (A319Q/Y415C) terpene synthases. FIG. 67A shows the product profile of Y415C and a double-mutant that combines mutations identified in a single-site library (E. coli s1030+pTS+pMBIS+pB2H in 10-ml TB media). The chromatogram shows extracted ions (m/z=204) scaled to injection size, which were determined from the peak area of an internal standard (20 μg/mL methyl abietate, m/z=316). Compound numbering refers to the scheme in FIG. 1A. FIG. 67B shows titers of the two major products of Y415C (β-himachalene and himachalol) for variants of GHS. The double mutant has a similar product profile to Y415C but exhibits a 57% lower titer. FIG. 67C shows spectinomycin resistance conferred by mutants of GHS. Images show the growth of E. coli harboring pMBIS, pTS, and pB2H on agar plates seeded from drops of liquid culture. The antibiotic resistance conferred by the double mutant matches that of Y415C, where the antibiotic resistance conferred by Y415C and the double mutant (A319Q/Y415C) are lower than the antibiotic resistance conferred by A319Q. Error bars in B denote standard deviation for n≥3 biological replicates.

FIGS. 68A-68D shows analysis of cellular toxicity and terpenoid production for improved mutants. FIG. 68A shows growth curves of strains expressing GHS or mutants with improved survival from a pET vector (T7 promoter) in the absence of pMBIS and pB2H. Specific growth rates for each mutant are shown in the plot's inset (h−1). FIG. 68B shows total terpene titers and product distributions for mutants in an evolutionary trajectory. FIG. 68C shows terpenoid titers for A319Q/Y415F. Analysis was focused on compounds with titers >2 μM (dashed line), noting that (i) accumulation of these terpenoids may result in 10-20× higher intracellular concentrations (confirmed in FIG. 4D) and (ii) detection of terpenoids with IC50s close to 20 μM has been demonstrated with B2H1. Compound labels that include a decimal point did not have high-confidence matches in the NIST MS library; these labels correspond to the observed retention time. When a compound could not be identified, its molecular weight was assumed to be 204 g/mol. FIG. 68D shows soluble fractions (soluble protein signal/total protein signal) of each GHS mutant expressed with a HiBit tag on a pET vector. Error bars in FIG. 68A and FIG. 68D denote standard error of n>3 biological replicates. Error bars in FIG. 68B and FIG. 68C denote standard deviation of n>3 biological replicates.

FIGS. 69A-69F shows the inhibition of PTP1B by three major products of GHS_{A319Q/Y415F}. FIG. 69A shows GC-MS chromatograms of purified fractions of three major products of GHS_{A319Q/Y415F}: FIG. 69B shows γ-humulene, β-himachalene, and himachalol. FIG. 69C and FIG. 69D shows inhibition of PTP1B activity on pNPP by γ-humulene, β-himachalene, and himachalol (colors as in FIG. 69B) in the presence of 10% (FIG. 69C) and 2% DMSO (FIG. 69D). FIG. 69E and FIG. 69F show absorbance at 405 nm at the start of the kinetic measurements from FIG. 69C and FIG. 69D: 10% DMSO (FIG. 69E) and 2% DMSO (FIG. 69F). All reactions in FIGS. 69C-69F include 50 nM PTP1B, 5 mM pNPP, and the indicated amount of DMSO and inhibitor. Error bars denote standard error for n≥3 independent measurements.

FIG. 70 shows the product profiles of Y415 mutant terpene synthases and double mutants compared to wild type. Screens uncovered several Y415 mutants that produce large amounts of himachalanes, particularly himachalol. The influence of Y415 was probed further by examining the profiles generated by Y415S and Y415T, which were generated by site-directed mutagenesis. Highlights: wild-type GHS (black label), mutants identified in high-throughput screens (grey), and rationally designed mutants (light grey). The chromatograms show extracted ions (m/z=204) scaled such that the height of the largest peak=1. Compound 25 could not be identified with high confidence (e.g., R-match >900) in the NIST MS library.

FIG. 71 shows an analysis of the antibiotic resistance conferred by rationally designed mutants. The spectinomycin resistance conferred by a Y415S and Y415T, which were designed after observing several hits with mutations at Y415. Images show the growth of E. coli strains harboring pMBIS, pTS, and pB2H on agar plates seeded from drops of liquid culture (X denotes a B2H system with a Y/F mutation in the peptide substrate). Top: representative data. Bottom: biological replicates. Both Y415S and Y415T fail to improve antibiotic resistance over A319Q alongside both active and inactive B2H systems. The reduced resistance exhibited by A319Q (relative to FIG. 2 and FIG. 64) may reflect slight differences in plate preparation.

FIGS. 72A-72B shows the mass spectrum (FIG. 72A) and the 1H NMR spectrum (FIG. 72B) of the purified sample described in FIG. 69.

FIG. 73 shows the mass spectrum of β-himachalene.

FIG. 74 shows the mass spectrum of himachalol.

FIG. 75 shows sites selected for site-saturation mutagenesis (SSM) from FIGS. 56A-65B. The table shown in FIG. 75 describes the highest scoring residues (see Eq. 3-1) and highlights the subset selected for SSM (top 6 entries in the table).

FIG. 76 shows titers of terpenoid-producing pathways. The table shown in FIG. 76 provides measurements of titers, including error and sample sizes, for strains containing various TS-specific pathways for terpenoid biosynthesis. In the depicted table, data from rows 3-5 correspond FIGS. 2B and 66B; data from row 4 correspond to FIGS. 2B, 67B, and 68B; data from rows 6 through 26 correspond to FIG. 62C; data from rows 27 through 34 and 36 through 38 correspond to FIG. 66C; data from rows 35 and 39-40 correspond to FIGS. 66B and 66C; data from rows 41-44 correspond to FIG. 67B; data from rows 45-50 correspond to FIG. 14B; data from rows 51-60 correspond to FIG. 68B; data from rows 61-69 correspond to FIG. 68C; data from rows 70-78 correspond to FIG. 62D; and data from rows 79-110 correspond to FIG. 64A.

FIG. 77 shows analysis of antibiotic resistance. The table shown in FIG. 77 describes the growth conditions (e.g., antibiotic concentrations in solid media) and experimental replicates used in our analysis of antibiotic resistance.

FIG. 78 shows kinetics of terpenoid-mediated inhibition. The table shown in FIG. 78 provides the discrete kinetic measurements made in this study, including error and exact sample sizes. Given the lack of detectable rates for low-substrate, high-inhibitor conditions, rates were not measured when substrate was absent. These points were treated as 0 in model fitting.

FIGS. 79A-79B shows bacterial two-hybrid (B2H) system that links protease inhibition to the expression of a gene of interest (GOI), with major components including (i) a kinase substrate fused to the omega subunit of RNA polymerase, (ii) a protease recognition (PR) site, (iii) a Src Homology 2 (SH2) domain fused to the 434 phage cI repressor, (iv) an operator for 434cI, (v) a binding site for RNA polymerase, (vi) the other subunits of RNA polymerase, and (vii) the GOI. Src kinase and protein tyrosine phosphatase 1B (PTP1B), which can be encoded by the same plasmid, activate or inhibit the SH2-substrate interaction through phosphorylation and dephosphorylation, respectively. Proteolysis of the PR site disrupts activation.

FIG. 79B shows related PR sites of the B2H system shown in FIG. 79A that were examined for HIVpro in reference to SEQ ID NO: 35, SEQ ID NO: 59, and SEQ ID NO: 60; for PLpro in reference to SEQ ID NO: 37 and SEQ ID NO: 61; for 3CL pro in reference to SEQ ID NO: 36, SEQ ID ON: 49, and SEQ ID NO: 50; and for USP7 pro in reference to SEQ ID NO: 54.

FIGS. 80A-80C shows performance of B2H systems that link PTP1B inactivation (C215S) to a luminescent output (LuxAB expression). FIG. 80A shows performance of B2H systems that link PTP1B inactivation (C215S) to a luminescent output (LuxAB expression) as compared to wild type. Protease recognition (PR) sites flanked by alanine (A) residues were added to the linker that connects MidT to the omega subunit of RNA polymerase. The addition of these PR sites reduces the dynamic range (e.g., the difference in fluorescence between active and inactive variants of PTP1B). FIG. 80B shows data from the use of a pBad plasmid and arabinose to titrate active and inactive proteases alongside the constitutively active B2H systems from FIG. 80A (e.g., the C215S systems). The systems with four-alanine linkers exhibited the highest dynamic range. FIG. 80C shows data from the use of the two-plasmid system from FIG. 80B to screen proteases against different PR sites. The dynamic range corresponds to the difference in luminescence between 0 and 0.2 w/v % arabinose. The white squares show the PR sites of the final B2H systems. FIG. 80D shows data from B2H systems generated by modifying the PTP1B-containing B2H system from FIG. 80A by (i) swapping in proteases for PTP1B, (ii) adding the highlighted PR sites from FIG. 80C, and, where necessary, (iii) adjusting the ribosome binding sites (RBSs) for protease genes. Images show the growth of E. coli harboring protease-specific B2H systems on agar plates seeded from drops of liquid culture (E. coli S1030+pB2H; LB agar, pH 7.5). In all FIGS. 80A-D, X denotes inactive variants of each protease: 3CLpro (H41A), HIVpro (D25N), USP7 (C223S), and PLpro (C111S). Data points denote the mean and standard error of n≥6 technical replicates.

FIGS. 81A-81B show performance of a plasmid-borne pathway for terpenoid biosynthesis. FIG. 81A shows a schematic of the plasmid-borne pathway for terpenoid biosynthesis: (i) pIUP, which converts isoprenol to farnesyl diphosphate (FPP), and (ii) pTS, which encodes a terpene synthase (TS). Genes: choline kinase (CK) and isopentenyl diphosphate isomerase (IDI) from S. cerevisiae, FPP synthase (FPPS) from E. coli, and isopentenyl phosphate kinase (IPK) from A. thaliana. (B) We transformed E. coli with three plasmids—a protease-specific B2H system (pB2H), pIUP, and pTS—and we used transformed strains to screen 37 phylogenetically distinct TSs for their ability to enhance spectinomycin resistance. Dashed boxes denote terpenoid pathways that conferred the greatest resistance for each B2H. FIG. 14B shows original data, and FIGS. 68A-68D show a re-screen of the hits for 3CLpro. In the rescreen, Q41594 (orange box) conferred the most consistent survival advantage for the 3CLpro system. (E. coli S1030+pB2H+pIUP_FPPS+pTS; LB agar with 2% glycerol, 10 mM isoprenol, and 50 μM IPTG, pH 7.0). FIG. 82A shows Sesquiterpene production by E3W205 and Q41594 in liquid culture. Chromatograms show total ion counts for full scans (m/z=50-350). (E. coli DH5α+pAM45+pTS; TB liquid with 500 μM IPTG).

FIGS. 82A-82F show inhibitors generated by Q41594 mutant terpene synthase. FIG. 82A shows GC-MS chromatograms of purified fractions of two major products of GHS_{Q41594} and GHS_{E3W205}. FIGS. 82B-82C show a representative number of α-bisabolol, generated by GHS_{Q41594} which has several stereoisomers. FIG. 82D shows the inhibition of 3CLpro by various bisabolenes (fluorogenic substrate=TSAVLQ_AFC) by IC50s. Data show the mean and standard error for n≥3 independent estimates (N.D., not determinable). FIG. 82E shows intracellular titers of the major products of GHS_{E3W205} and GHS_{Q41594} in LB or TB liquid media: α-bisabolol (1) and β-bisabolene (2). Highlight includes the titer of α-bisabolol produced by Q41594 (12.9+/−3) in TB media, where the * symbol represents no detectable product. Data show the mean and standard deviation for n≥3 biological replicates. (E. coli S1030+pB2H_3CL+pIUP_FPPS+pTS; LB liquid with 50 μM IPTG and 10 mM isoprenol; TB liquid with 500 μM IPTG and 50 mM isoprenol. FIG. 82F shows growth curves for E. coli harboring pTS grown in LB liquid media. Q41594 reduces the specific growth rate. Data show the mean and standard error for n≥3 biological replicates. (E. coli S1030+pTS; LB liquid with 50 μM IPTG).

FIGS. 83A-83D show performance of a B2H system with phosphorylated peptide (MidT) that binds to a Src homology 2 (SH2) domain, activating transcription of a gene of interest (GOI). PTP1BX denotes catalytically inactive PTP1B (e.g., C215S). FIG. 83A shows a schematic of the B2H system. FIG. 83B shows a monobody (HA4) binds to an SH2 domain, activating transcription of a GOI. FIG. 83C shows versions of B2Hs from FIGS. 83A-B with LuxAB as the GOI. A pBad plasmid was used to titrate HIVpro and 3CLpro alongside the B2H of FIGS. 83A-B. For both systems, 3CLpro reduced luminescence, but the background signal was much higher for the B2H of FIG. 83B. Data points denote the mean and standard error of n≥6 technical replicates. FIG. 83D shows modified B2Hs of FIGS. 83A-83B that were modified by swapping out LuxAB for SpecR. Images show the growth of E. coli harboring protease-specific B2H systems on agar plates seeded from drops of liquid culture. The “X” denotes an inactive variant of HIVpro (D25N); the “*” denotes inactive PTP1B for the B2H of FIG. 83A or a missing SH2 domain for B2H of FIG. 83B.

FIGS. 84A-84B show a B2H system that links USP7 inactivation to the expression of a gene for spectinomycin resistance (SpecR) in the presence of a protease inhibitor, wherein the linker between the MidT and RPω is modified to contain the sequence AAAAUbiquitinAAAA (SEQ ID NO: 54). FIG. 84A shows a B2H system that links USP7 inactivation to the expression of a gene for spectinomycin resistance (SpecR). The peptide stretch that links the kinase substrate to the omega subunit of RNA polymerase contains a protease recognition (PR) site for USP7. FIG. 84B shows images depicting the growth of E. coli harboring the initial USP7-specific B2H system on agar plates seeded from drops of liquid culture. The “X” denotes an inactive variant of USP7 (C223S). The initial RBS and PR tested with the system afforded a dynamic range comparable to other protease-specific B2H systems.

FIGS. 85A-85C show a B2H system that links HIVpro inactivation to the expression of a gene for spectinomycin resistance (SpecR). FIG. 85A shows that the peptide stretch that links the kinase substrate to the omega subunit of RNA polymerase contains a protease recognition (PR) site for HIVpro in reference to SEQ ID NO: 35 or SEQ ID NO: 36. Aspects that were evaluated were (i) four ribosome binding sites (RBSs) for HIVpro and (ii) three PR sites (including no PR). FIG. 85B shows images depicting the growth of E. coli harboring HIVpro-specific B2H systems on agar plates seeded from drops of liquid culture. The “X” denotes an inactive variant of HIVpro (D25N). The inclusion of a PR (bottom) improves the sensitivity of E. coli to HIVpro expression (e.g., it reduces spectinomycin resistance). RBSs with different TIRs also affect this sensitivity, but with no obvious trends. FIG. 85C shows the RBS with an estimated TIR of 20k yields the highest dynamic range (e.g., a greater difference in spectinomycin resistance between active and inactive variants of HIVpro). The RBS and KARVL*AEAM were selected to construct additional embodiments of the B2H system.

FIGS. 86A-86B show B2H system that links 3CLpro inactivation to the expression of a gene for spectinomycin resistance (SpecR). FIG. 86A shows that the peptide stretch that links the kinase substrate to the omega subunit of RNA polymerase contains a protease recognition (PR) site for 3CLpro in reference to SEQ ID NO: 50. To modulate expression of 3CLpro, two ribosome binding sites (RBSs) were evaluated with different translation initiation rates (TIRs). FIG. 86B shows images depicting the growth of E. coli harboring 3CLpro-specific B2H systems on agar plates seeded from drops of liquid culture. The “X” denotes an inactive variant of 3CLpro (H41A). The RBS with the higher TIR confers a higher dynamic range (e.g., difference in spectinomycin resistance between active and inactive 3CLpro). The RBS was selected for the final system.

FIGS. 87A-87C show a B2H system that links PLpro inactivation to the expression of a gene for spectinomycin resistance (SpecR). FIG. 87A shows that the peptide stretch that links the kinase substrate to the omega subunit of RNA polymerase contains a protease recognition (PR) site for PLpro in reference to SEQ ID NO: 50. To modulate expression of PLpro, a library of 32 ribosome binding sites (RBSs) was evaluated with translation initiation rates (TIRs) ranging from 52 to 42,0000. FIG. 87B shows the results of a drop-based screen of 116 B2H systems with different RBSs for PLpro. The 116 systems contain a maximum diversity of 32. Three RBSs that conferred sensitivity to spectinomycin were selected were RBSs from sample 2, 13, and 18. Controls (bottom): B2H systems with active and inactive 3CLpro (H41A). FIG. 87C shows images depicting the growth of E. coli harboring PLpro-specific B2H systems with RBSs 2, 13, and 18 from B. The “X” denotes an inactive variant of PLpro (C111S). RBS 18, which confers the greatest sensitivity to spectinomycin, was selected for additional embodiments of the B2H.

FIG. 88 shows images depicting the growth of E. coli harboring protease-specific B2H systems on agar plates seeded from drops of liquid culture. The PTP1B-specific B2H, which guided the design of the protease systems, serves as a reference. The “X” denotes inactive variants of each enzyme: PTP1B (C215S) (with reference to SEQ ID NO: 6), 3CLpro (H41A) (with reference to SEQ ID NO: 69), HIVpro (D25N) (with reference to SEQ ID NO: 63), USP7 (C223S) (with reference to SEQ ID NO: 65), and PLpro (C111S) (with reference to SEQ ID NO: 67).

FIG. 89 shows the spectinomycin resistance conferred by different terpenoid pathways. Images show the growth of E. coli strains harboring protease-specific B2H systems, pIUP_FPPS, and pTS on LB agar plates (e.g., LB agar with 2% v/v glycerol, 10 mM isoprenol, 50 μM IPTG, and pH 7.0 supplemented with antibiotics) seeded from drops of TB liquid culture. This raw data was used to create FIG. 81B.

FIGS. 90A-90B show the spectinomycin resistance conferred by terpenoid pathways that emerged as hits in our initial screen (FIG. 81 and FIG. 89). These images show the growth of E. coli strains harboring pB2H_3CLpro, pIUP_FPPS, and pTS on LB agar plates (e.g., LB agar with 2% v/v glycerol, 10 mM isoprenol, 50 μM IPTG, and pH 7.0 supplemented with antibiotics) seeded from drops of liquid culture. FIG. 90A and FIG. 90B show that Q41594 conferred the most prominent survival advantage. FIG. 90B shows data from a repeat test as described with respect to FIG. 90A with biological replicates, only Q41594 improved antibiotic resistance over an empty vector (e.g., “Empty”, the pTS plasmid with no TS gene). In general, Q41594 yielded the most consistent survival advantage over all assays. Note: “Empty” denotes a pTS plasmid with no TS gene.

FIGS. 91A-91B shows the products and product profiles of terpene synthases that enhanced or failed to enhance the antibiotic resistance of E. coli harboring the 3CLpro-specific B2H (FIG. 3). FIG. 91A provide chromatograms that show total ion counts for full-scans (m/z=50-350). The * symbol denotes hits from the initial screen (FIG. 81B). O65504, which was a hit not examined in this figure, is a well-characterized γ-humulene synthase from Abies grandis; it produces a mixture of products. FIG. 91B shows the major products identified in FIG. 91A. Q41594, which conferred a consistent survival advantage in our repeat tests (FIG. 90), produces α-bisabolol.

FIGS. 92A-92F show products and product profiles of terpene synthases that enhanced antibiotic resistance. FIG. 92A shows the inhibition by 3CLpro by several bisabolenes generated by TSs examined. FIG. 92B shows a plot depicting the percent activity (e.g., the percent of the inhibitor-free initial rate on a model peptide) that remains after incubation with different concentrations of bisabolenes from FIG. 92A (colored as in FIG. 92A). The inhibition of 3CLpro by β-bisabolene and β-bisabolol was too weak to permit accurate IC50 estimates (≤50% inhibition at 1000 μM terpenoid). FIGS. 92C-92F show dose-response curves used to estimate IC50s for the four most inhibitor compounds. Data denote the mean, standard error, and independent measurements for n≥3 technical replicates.

FIGS. 93A-93B show products and their performance in conferring antibiotic resistance. FIG. 93A shows bisabolene products of previously characterized terpene synthases (TSs) not included in the first screen A0A118JX19 (in reference to SEQ ID NO: 15), A0A1L7NYG3 (in reference to SEQ ID NO: 17), J7LH11 (in reference to SEQ ID NO: 19), A0A386JV86 (in reference to SEQ ID NO: 70), D2YZP9 (in reference to SEQ ID NO: 9), WP_035857999 (in reference to SEQ ID NO: 71), and O81086 (in reference to SEQ ID NO: 72). FIG. 93B shows the spectinomycin resistance conferred by different bisabolene-producing terpene synthases. These images show the growth of E. coli strains harboring pB2H_3CLpro, pIUP_FPPS, and pTS on LB agar plates (e.g., LB agar with 2% v/v glycerol, 10 mM isoprenol, and 50 μM IPTG at pH 7.0 supplemented with antibiotics) seeded from drops of liquid culture. Several TSs from the first screen were included that can generate bisabolenes: including Sesquiterpene synthase 14b (Uniprot ID: G8H5N1), β-Bisabolene synthase in reference to SEQ ID NO: 11, and Amorpha-4, 11-diene synthase (Uniprot ID: Q9AR04), and a protein that makes amorphadiene, Taxadiene synthase (UniProt ID: Q41594). Numbers on the y axis of FIG. 93B denote UniProt ids except for WP_035857999 (NCBI).

FIGS. 94A-94G show products and their performance conferring survival advantage. FIG. 94A shows the product profiles of a subset of terpene synthases (TSs) that generate bisabolene. Chromatograms show total ion counts for full-scans (m/z=50-350). J7LH11 (*) conferred a survival advantage in our second screen (FIG. 93). FIG. 94B shows the major products identified in FIG. 94A. FIGS. 94C-94G show TSs and their major products, such as A0A386JV86 ((Z)-α-bisabolene) in FIG. 94C, WP_035857999 ((Z)-γ-bisabolene) in FIG. 94D, A0A118JXI9 (α-bisabolol) in FIG. 94E, J7LH11 (α-bisabolol), and (G) G8H5N1 (α-bisabolol) in FIG. 94F.

FIG. 95 shows the 1H NMR spectrum α-bisabolene at 300 MHz, CDCl3

FIGS. 96A-96B show GC-MS standard curves for two products. FIG. 96A shows the GC-MS standard curves for bisabolene. FIG. 96B shows the GC-MS standard curves for bisabolol quantification.

FIG. 97 shows the standard curve links the concentration of AFC to the fluorescence of this molecule (λex=400 nm, λem=505 nm) in 100 μL of buffer (25 mM HEPES, pH=7.3) in a 96-well plate.

FIGS. 98A-98D show structures of various compounds identified with the systems disclosed herein and their performance. FIG. 98A shows structures of amorphadiene (AD) as well as well-studied allosteric (BBR) and competitive (TCS401) inhibitors. FIG. 98B shows an X-ray crystal structure of PTP1B bound to AD (PDB entry 6W30) with the binding sites for BBR and TCS401 overlaid for reference (PDB entries 6W30, 1T4J, and 5K9 W). AD and BBR bind to the allosteric site, which includes residues from the α3, α6, and α7 helices. TCS401 binds to the active site, which is flanked by the WPD and P-loops. FIG. 98C shows fluorescence-based binding isotherms for BBR measured in the presence and absence of either AD or TCS401. Similar levels of binding by AD and TCS401 were ensured by using concentrations that produced similar levels of inhibition (˜50%). Binding parameters (+SE) included ΔF=(ΔFmax*L)/(Kd+L), where Kd=10.1±2.7 μM and ΔFmax=227000±13000 for BBR alone, where Kd=13.1±3.8 UM and ΔFmax=195000±12000 for BBR with AD, and where Kd=31.0±2.8 UM and ΔFmax=94000±2000 for BBR with TCS401. The insensitivity of the BBR binding isotherm to the presence of AD suggested that the two inhibitors can bind simultaneously. Error bars denote standard error for n=3 technical replicates. FIG. 98D shows melting temperatures determined with differential scanning fluorimetry. The data indicate that BBR and Ertiprotafib destabilize PTP1B, while AD and TCS401 do not. Error bars denote standard deviation for n=3 technical replicates.

FIG. 99 shows the kinetics of inhibition for various experiments as described with respect to FIGS. 82D and 92B-92F.

FIG. 100 shows titers of natural product pathways with respect to FIG. 82E, where sample size indicates the number of biological replicates used in the study (e.g., the number of distinct bacterial colonies grown up for the study), experimental sets indicates the number of times the experiment was run (e.g., with the indicated number of biological replicates), and N.D. stands for “not detected”.

FIG. 101A-101B show a schematic and results of the B2H system disclosed herein according to some embodiments. FIG. 101A shows a schematic of the inverted bacterial-two hybrid system. In this embodiment, the kinase activity enables SH2/MidT binding, which subsequently turns on expression of a repressor protein R. R binds to an operator sequence within a constitutive promoter expressing green fluorescent protein (GFP). The function of this system can be observed in DH10BΔRpoω cells (e.g., DH10B cells with the gene for the omega subunit of RNA polymerase knocked out) harboring either (i) the system depicted in FIG. 101 (“inverted B2H”), (ii) system depicted in FIG. 101 with the tyrosine residue of the MidT substrate mutated to a phenylalanine (“inverted B2Hx”), or (iii) a system lacking GFP. A composite image of these cells show that the inverted B2Hx system produces much more fluorescence than inverted B2H or “no GFP” systems, demonstrating phosphorylation-dependent transcriptional repression of the GFP. FIG. 101B depicts biological triplicate data of DH10BΔRpoω cells with plasmid-borne versions of B2H systems from FIG. 101A, where cells are seeded on agar plates from drops of liquid culture.

FIGS. 102A-102B shows a schematic of the B2H system encoding antibiotic resistance according to some embodiments herein. FIG. 102A shows an inverted B2H system that links kinase activity to the repression of a gene for spectinomycin resistance (inverted B2H). FIG. 102B shows DH10BΔRpoω cells harboring inverted B2H systems with different combinations of SpecR promoters (bla or J23110) and repressors (SrpR, AmeR, BetI, PsrA, PhiF). In all constructs, repressors were paired with their cognate operator sequences. The “No operator” construct contains an HlyII repressor (R) with no operator sequence in the SpecR promoter. This data suggests that the inverted two-hybrid system requires changes in expression of the repressor gene and/or the resistance gene.

FIG. 103 shows GFP signal from the B2H system. FIG. 103 is a histogram showing flow cytometry measurements of cells harboring three B2H systems: a negative control with no GFP (gray), an inverted B2H (light gray, an inverted two-hybrid system that links kinase activity to the repression of a gene for spectinomycin resistance), inverted B2Hx (dark gray, inverted B2H with the MidT Y/F mutation). Cells were gated to remove debris and to select for single cells. At least 10,000 events were collected for each measurement.

FIG. 104 shows a Src Kinase Inverted B2H system as an example of the system disclosed herein against a library of individual terpene synthase enzymes. FIG. 104 shows a schematic of an inverted B2H system in which fluorescence (GFP) increases in the presence of an inhibitor. In this embodiment, the terpene synthase synthesizes an inhibitor that blocks Src activation of the repressor, enabling expression of GFP. On agar plates containing spots of E. coli cells that contains (i) the inverted B2H system depicted in FIG. 104, (ii) a pathway that produces farnesyl pyrophosphate (pAM45), and/or (iii) a terpene synthase, fluorescent spots can be used to identify terpene synthases the produce inhibitors of Src kinase. Certain plates may contain a catalytically inactive variant of amorphadiene synthase in place of the terpene synthase.

FIGS. 105A-105B shows schematics of transcriptional systems described herein. FIG. 105A depicts the B2H with T7 as the GOI referred to as “T7opt”. In this embodiment, the B2H system detects phosphatase activity. In this embodiment, both B2H binding partners (cI-SH2, rpoZ-sub) are constitutively expressed from the prol promoter. In this embodiment, Src Kinase, Cdc37, and PTPB1 are expressed constitutively from the prod promoter. In other embodiments, the PTP1B is not expressed. In this embodiment, the T7 RNAP is expressed when PTPB1 is inactivated (by C215S inactivating mutation). FIG. 105B shows an auxiliary pET16b vector that provides GFPuv under control of the T7 operator. In this embodiment, the auxiliary pET16b vectors can be paired with expression of T7 RNAP via successful B2H partner binding to enable expression of GFPuv.

FIGS. 106A-106B shows 96 individual colonies expressing RBS variants (L2-GOI RBS library) quantified by fluorescence. OD600 quantifies potential toxic effects of T7 RNAP expression, which is differentially modulated by the different RBSs.

FIG. 107 shows quantification of fluorescence from an embodiment of a B2H systems. In this experiment, the cells contain (i) a first plasmid with a B2H system in which the GOI is a gene for T7 RNA polymerase, and the T7 RNA polymerase is modulated by an RBS chosen in the screen described by FIG. 106, and (ii) a secondary plasmid with a gene for a green fluorescent protein (GFP) under control of a T7 promoter such that expression of the T7 RNA polymerase from the first plasmid results enhanced GFP expression. The two versions of the B2H systems depicted contain a WT PTP1B or a mutated PTP1B (C215S).

FIG. 108 shows quantification of fluorescence from an embodiment of a B2H systems. In this example, the first plasmid encodes a B2H system that contains a gene for T7 RNA polymerase as the GOI and lacks genes for both (i) a PTP (ii) MidT fused to the omega subunit of RNA polymerase (RpoZ), the second plasmid encodes a gene for MidT fused to the omega subunit of RNA polymerase, and the third plasmid encodes a gene for GFP under control of a T7 promoter. Variants include versions in which the second plasmid contains MidT alternatives: substrate, mutated MidT (Y/F substitution), or WT MidT.

FIGS. 109A-109D shows quantification of luminescence from various embodiments of a B2H system. FIG. 109A shows the luminescence output form a B2H embodiment including a DNA Binding Protein CymR-AM, a DNA binding protein fused to an SH2 domain, and RpoZ fused to an HA4 monobody. In this embodiment, CymR is compared to a cI embodiment described previously. FIG. 109B shows the luminescence output form a B2H embodiment including a DNA Binding Protein PhlF, a DNA binding protein fused to an SH2 domain, and RpoZ fused to an HA4 monobody. In this embodiment, Ph 1F is compared to a cI embodiment described previously. FIG. 109C shows the luminescence output form a B2H embodiment including a Lambda Phage DNA Binding Protein Cro, a DNA binding protein fused to an SH2 domain, and RpoZ fused to an HA4 monobody. In this embodiment, DBP is compared to other embodiments described previously. In some embodiments, a system uses a phosphorylation-independent HA4-SH2 interaction instead of a phosphorylation-dependent MidT-SH2 interaction. FIG. 109D shows Cro and different numbers of operator and protein architecture. In place of OR1/OR2 operators for cI in the original system, either one or two copies of OR3 (Cro's operator) are encoded.

FIG. 110 shows an embodiment of a B2H system using iLID-SsrA/SspB binding partners. In this embodiment, red fluorescent protein is the GOI. In this embodiment, transcriptional activity was induced exposing cultures to 490 nm blue light for 24 hours to enable SsrA-SspB binding and localizing rpoZ or rpoA to the promoter site. In some embodiments, the SsrA-SspB binding is to the N-terminal region, which is a truncate portion of the alpha subunit of the RNA polymerase.

FIGS. 111A-111B shows an example of a next-generation sequencing from cells expressing a B2H system. FIG. 111A shows an example of a next generation sequencing from cells expressing a B2H system that links PTP1B inactivation to the expression of gene for spectinomycin resistance and (ii) a terpenoid pathway, comprising an isoprenoid pathway (pAM45), a terpene synthase, and a cytochrome P450 (CYP2A6). In some embodiments, the cells are seeded on agar plates with different concentrations of spectinomycin (μg/ml) NGS was used to assess the population fraction associated with different terpenoid pathways. This B2H system includes a “full-length” version of PTP1B (1-405). FIG. 111B shows an example of an analogous experiment in which the B2H linked TCPTP inactivation to the expression of a gene for spectinomycin resistance. This B2H system includes a “full-length” version of TC-PTP (1-287).

FIGS. 112A-112C shows a schematic of example embodiments of the workflow including plasmid construction, target enzyme combinations, and analysis. FIG. 112A shows an exemplary strategy for building plasmids that contain different combinations of terpene synthases and terpenoid-functionalizing enzymes, such as a P450. FIG. 112B shows an exemplary workflow for screening different target enzyme combinations for their ability to confer a survival advantage in the presence of a B2H system and the use of NGS to calculate the enrichment associated with different TS/P450 combinations. FIG. 112C shows an embodiment of a workflow for using PCR amplification to prepare for NGS and the subsequent use of NGS to demultiplex the results of a large screen. In some embodiments, the TS region (with P450 ID barcodes) may be amplified using universal TRC plasmid primers. In some embodiments, the oligo-based barcodes may be added to identify specific B2H conditions. In some embodiments, the barcodes may be used to bin data. In some embodiments, the number of reads for each terprene synthase with and without Spec selection may be counted to identify terpene synthases are enriched.

FIGS. 113A-113B shows embodiments of selection experiments using different B2H systems. FIG. 113A shows the population fraction belonging to each strain. The left panel in FIG. 113A show the strain containing B2H contains both a gene for GFP and a B2H system that links PTP1B inactivation to the expression of gene for spectinomycin resistance. The second strain (B2H*) contains an empty plasmid lacking GFP and a B2H system with a catalytically inactive (C215S) mutant of PTP1B. High concentrations of spectinomycin and longer growth times appear to enrich for the B2H* system. The right describes the complementary experiment in which the first strain (B2H) has both GFP and a B2H system with the C215S mutant of PTP1B, and the second strain has both an empty vector and a B2H system. In both experiments, the numbers above the bars show the population fraction of B2H* divided by the population fraction of B2H. FIG. 113B shows another embodiment quantifying colony count frequency comparing the B2H system to the B2Hx, a B2H system in which a Y/F mutation in the substrate domain (MidT) prevents its phosphorylation at residue that helps it bind to the receptor and amorphadiene synthase.

FIGS. 114A-114B shows a method and example of using next-generation sequencing to identify target enzymes that confer a survival advantage under selective conditions. FIG. 114A depicts a schematic describing the construction and screening of a target enzyme mutant library against a protein target of interest in a B2H experiment. FIG. 114B shows the results of an exemplary application of this workflow for mutagenesis and screening of a target enzyme. Target enzyme mutants identified from next generation sequencing are ranked. The mutants with the highest enrichment comparing selected versus unselected population are shown.

FIGS. 115A-115B shows an example embodiment of an NGS workflow. FIG. 115A shows a schematic of an NGS workflow for screening of a target enzyme. FIG. 115B shows an embodiment of the workflow depicted in FIG. 115A. was carried out in triplicate using a PTPRC-based B2H detection system. Genes with enrichment values >0 are labeled. Error bars in FIG. 115B denote standard error of N=3 biological replicates.

FIGS. 116A-116D show schematics of example B2H embodiments disclosed herein. FIG. 116A shows an embodiment of a system using a phosphorylation-independent HA4-SH2 interaction instead of a phosphorylation-dependent MidT-SH2 interaction. DNA binding protein cI is fused to an SH2 binding domain. The omega subunit to E. coli RNA polymerase is fused to the monobody HA4 domain to create a constitutively active transcriptional system. cI is able to bind cooperatively to its operators OR2 and OR3 and thereby localize RNA polymerase to the promoter via B2H interactions, transcribing reporter luxAB. FIG. 116B shows an embodiment of the repressor CymR and its cognate operator CuO. FIG. 116C shows an embodiment of the binding protein, PhlF, and its cognate operator PhlO. FIG. 116D shows an embodiment of the Cro repressor and its operator OR3.

FIGS. 117A-117D show schematics of example B2H embodiments disclosed herein. FIG. 117A shows an embodiment disclosed herein of a system using a phosphorylation-independent HA4-SH2 interaction instead of a phosphorylation-dependent MidT-SH2 interaction. FIG. 117B shows an embodiment of the Cro repressor and OR3 replace cI and its cognate operators OR1 and OR2. FIG. 117C shows an embodiment of one permutation of this system encodes another copy of OR3 upstream of the reporter gene promoter region. FIG. 117D shows an embodiment of a single chain Cro repressor was encoded which links two Cro proteins via a flexible 8 amino acid linker. This step was theorized to overcome the thermodynamic step associated with homodimer formation necessary for DNA binding.

FIGS. 118A-118B show schematic embodiments of a B2H system reliant on blue light. FIG. 118 A shows an embodiment of a B2H system reliant on a Blue Light inducible dimer. In blue light the LOV2 domain is excited causing disordering of the Ja helix which allows the SsrA peptide to be uncaged. The SsrA peptide can then bind with its partner SspB and allow localization of RNAP to the promoter via rpoZ recruitment. FIG. 118B shows an embodiment that uses the same system as FIG. 118A with a modified N-terminal domain of the alpha subunit (1-248) fused to the LOV2-SsrA domain. This system was assessed along with the 118A to determine the effect of the Alpha subunit on transcriptional activation.

### DETAILED DESCRIPTION

Disclosed herein are systems, methods, and compositions for the discovery of bioactive molecules with therapeutic potential that modulate the activity of a target enzyme. The disclosure also provides systems, methods and compositions for directed evolution of metabolic pathways that produce bioactive molecules that modulate target enzyme function. The systems and methods disclosed herein have been optimized for high-throughput screens of bioactive modulators of a target enzyme (e.g., terpenoids) that, in some cases, mimic or recreate natural processes of diversification and selection. For instance, the methods and systems for high-throughput screens may involve large numbers of metabolic pathways, target enzymes, or both, thereby increasing the diversity and number of bioactive molecules that can be discovered. In some embodiments, the system comprises one or more expression systems including without limitation (i) a two-hybrid system that, when expressed in a cell, links a detectable output (e.g., luminescence or cell growth) to the modulation of a target enzyme (e.g., therapeutic target), and (ii) a metabolic system that enables the biosynthesis of structurally varied bioactive molecules that modulate a target enzyme (e.g., potential therapeutic agent). In some embodiments, the cell is a microorganism, such as a bacterial cell (e.g., E. coli). In some embodiments, the detectable output is amplified by linking the activity of the target enzyme to a gene of interest (GOI) encoding an enzyme that drives expression of a detectable polypeptide, such as a fluorescent or bioluminescent polypeptide.

Some aspects of this disclosure provide systems, methods and compositions for identifying bioactive molecules that modulate the activity of proteases, protein phosphatases (e.g., protein tyrosine phosphatase), or combinations thereof. In some embodiments, the systems, methods and compositions described herein are capable of identifying bioactive molecules with therapeutic potential that modulate the activity of a various proteases utilizing a specific variety of the two-hybrid system that contains a protease cleavage recognition motif that, when cleaved by the protease, disrupts transcription of the GOI. In some embodiments, target enzymes may be an enzyme of a pathogen. For example, a target enzyme may be a functional protein of a virus (e.g., viral protease), such that an implementation of the systems and methods disclosed herein is used to discover a bioactive molecule (e.g., therapeutic molecule) that targets the functional protein of a virus. Similarly, in some embodiments, the target enzyme may be a functional protein of a bacterial pathogen, a prion pathogen, or any one of various pathogens where the functional protein is tied to the infectivity, severity, and/or progression of a disease associated with the pathogen. Utilizing the systems and methods disclosed herein may accelerate the discovery process of therapeutic molecules.

Some aspects of this disclosure provide systems, methods and compositions for identifying novel synthases that produce the bioactive molecules disclosed herein. In some embodiments, the novel synthases are terpene synthases or non-ribosomal peptide synthetases. The present disclosure provides numerous modified synthases that have undergone single site mutagenesis (SSM) to improve production of bioactive molecules of interest. In some embodiments, modified terpene synthases disclosed herein produce increased diversity novel terpenoids with therapeutic potential.

Aspects of this disclosure also provide cells (e.g., microorganisms) that are configured to guide the discovery and biosynthesis of the bioactive molecules as novel targeted therapeutics. In some embodiments the cells are semi-synthetic. In some embodiments the cell comprises the one or more expression systems disclosed herein. In some embodiments, the cells produce the bioactive molecules, such as metabolic products or modulators (e.g., activators or inhibitors) of a target enzyme, disclosed herein. In some cases, discovered metabolic products may exhibit single-digit micromolar half maximal inhibitory concentrations (IC₅₀s) or inhibitor constants (Kᵢs), or unusual modes of inhibition, or a combination thereof.

Drug design is an exceedingly difficult problem. Despite advances in structural biology and computational chemistry, the design of molecules that bind tightly to specific disease-relevant proteins can still be extremely difficult. Some drug development processes may begin with screens of large molecular libraries. A molecule, once identified, may be synthesized in quantities sufficient for subsequent analysis, optimization, and clinical evaluation—which is a challenging feat. The economics of pharmaceutical development for infectious diseases may disincentivize costly discovery efforts until after an outbreak has occurred—which may constrain the time available to search a given chemical space accessible with some screening methodologies.

Meanwhile, nature has endowed living systems with the catalytic machinery to build an enormous variety of biologically active molecules. These living systems evolved to synthesize various biologically active molecules to carry out important metabolic and ecological functions (e.g., the phytochemical recruitment of predators of herbivorous insects) which sometimes exhibit useful medicinal properties in humans. Over the years, screens of environmental extracts and natural product libraries—augmented, on occasion, with combinatorial (bio) chemistry—have uncovered a diverse set of therapeutics, from aspirin to paclitaxel. Unfortunately, these screens may be resource intensive, limited by low natural titers, and largely subject to serendipity. Bioinformatic tools, in turn, have permitted the identification of biosynthetic gene clusters, where co-localized resistance genes can reveal the biochemical function of their products. The therapeutic applications of many natural products, however, differ from their native functions, and many biosynthetic pathways can, when appropriately reconfigured, produce entirely new and, perhaps, more effective therapeutic molecules. Methods for identifying and evolving natural products that solve specific, therapeutically relevant challenges remain largely undeveloped; as a result, the biomedical potential of these molecules—and the enzymes that make them—has yet to be fully realized.

The system disclosed herein, in some embodiments, comprise a two-hybrid system (e.g., bacterial two-hybrid (B2H) system) that, when transfected into a cell, links survival or a detectable output of the cell to production of modulator of a target enzyme (e.g., therapeutic target) encoded by the two-hybrid system. In some embodiments, the system also comprises a one or more exogenous nucleic acid molecules encoding a metabolic pathway and a synthase responsible for expressing the bioactive molecules in the cell that modulate the target enzyme. In some embodiments, the cell is a genetically encoded microorganism (e.g., E. coli) engineered to express the two-hybrid system, the metabolic system, and the synthase under conditions sufficient to guide the cell to assemble various bioactive molecules that modulate the intended target enzyme. This approach has numerous important benefits over traditional drug discovery processes, including, but not limited to: (i) it can enable rapid, fermentation-based scale up for compound optimization, preclinical studies, and early human trials, and, thus, promises to accelerate the pace—and reduce the cost—of therapeutic development; (ii) it does not necessarily presuppose a specific molecular structure and thus facilitates the identification of nonintuitive relationships between modulators (e.g., inhibitors) and target enzymes (e.g., drug targets); (iii) it does not necessarily require the specification of a single binding site and thus permits the discovery of new sites; (iv) it can use cellular machinery (e.g., chaperones) to stabilize full-length drug targets; (v) it permits the construction of structurally varied leads, or “backups”, that can mitigate risk in drug development; (vi) it is compatible with DNA barcoding technology and next-generation sequencing and, thus, permits multiplexing across many pathways and many targets. The economics of the system are well suited for multi-target discovery campaigns designed to produce broad set of new, synthetically tractable lead compounds before a pandemic has occurred (or rapidly after it begins). The inventive concepts disclosed herein build on certain aspects of the systems disclosed in U.S. patent application Ser. Nos. 17/141,321 and 17/859,509, each of which is hereby incorporated by reference in its entirety.

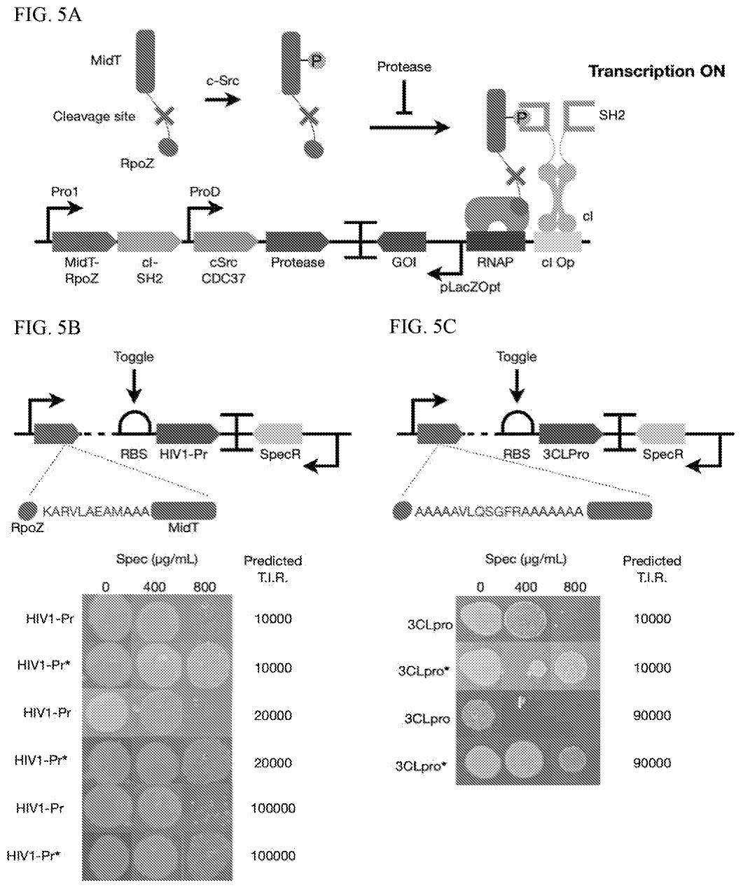

Provided herein, in some aspects, are genetically-encoded systems that have been modified to identify modulators of new target enzymes (e.g., therapeutic targets), such as proteases. In some embodiments, the two-hybrid system, the metabolic pathway, the synthase, or any combination thereof, of the genetically-encoded systems is modified. For example, referring to FIG. 5A, the two-hybrid system may be engineered to preferentially select cells that produce inhibitors of proteases by engineering the transcriptional machinery to turn on expression of a gene of interest (GOI) (e.g., reporting gene) that conferring a survival advantage during the selection process only when the cell produces a product that inhibitor of proteolysis of a cleavage site engineered in a linker coupled to a transcriptional activator of the two-hybrid system. Subcomponents of the two-hybrid system can also be modified extensively, as disclosed elsewhere herein, such as for example, the linker comprising the cleave site to enhance a survival advantage.

In some aspects, the systems are engineered to produce natural and unnatural protease inhibitors of a particular drug target by harnessing the endogenous biosynthetic pathways of the cell. In some embodiments, the proteases are human proteases, viral proteases, or a combination thereof. Discovery of viral protease inhibitors may be relevant to preventing or treating disease or conditions associated with pathogenic infections by disrupting the function(s) of a given virus (e.g., HIV-1 protease (HIV-1Pr) and 3-chymotrypsin-like protease (3ClPro) from SARS-COV-2). In some embodiments, the human proteases comprise Ubiquitin-specific-processing protease 7 (USP7). Discovery of human protease inhibitors may be relevant to preventing or treating diseases or a conditions associated with the overactivity or overexpression of proteases, including for example, vascular disease, cancer, and others.

Further, the optimal design of each protease system and workflow disclosed herein is adaptable to the development of similar tools for the discovery of modulators of other types of therapeutic targets. Using the evolved 74 terpenoid pathways and identified several enzyme combinations that show altered resistance phenotypes (implying biosynthesis of protease inhibitors). The system, which may encompass a bacterial two-hybrid system may enable the detection of biosynthetically accessible small molecules that inhibit proteases and other potential therapeutic targets.

Optimization of the systems disclosed herein to identify novel modulators of proteases has a profound implications for treating difficult-to-treat disease or conditions associated with protease activity. Proteases are centrally important to many biochemical processes and have provided a rich set of targets for treating human diseases. These enzymes, which catalyze the hydrolysis of peptide bonds, coordinate the dynamic remodeling—and functional rewiring—of the complex protein systems that underlie blood clotting, repair, and viral assembly, among other biochemical feats. Over the years, proteases have emerged as important targets for other viral diseases—notably, hepatitis C and Coronavirus disease of 2019 (COVID-19)—as well as cardiovascular disorders and cancer. Despite their therapeutic promise, proteases often evolve resistance mutations, which can emerge early in clinical trials, and remain subject to the same slow development timelines that plague other drugs. New approaches for discovering protease inhibitors could help address resistance mutations and accelerate drug development.

Natural products are a longstanding source of pharmaceuticals and bioactive compounds, including protease inhibitors, but have proven challenging to screen in high-throughput assays. Their low natural abundance and complex biological matrices (e.g., multicomponent extracts) tend to complicate compound detection and dereplication, while their chemical structures, which often include multiple stereocenters, tend to slow scale-up and hit optimization. Advances in microbial genetics and bioinformatics have led to an explosion of new biosynthetic gene clusters (BGCs) and uncovered enzymes capable of adding biochemically nonstandard functionalities (e.g., terminal alkynes, halogens, and hydrazines). The structures and biological activities of biosynthetic compounds, however, remain challenging to predict from sequence data alone, and functional characterization typically requires laborious extraction and purification steps.

The genetically encoded microorganisms disclosed herein, which are equipped with the systems disclosed herein, offer a promising means of accelerating the discovery of pharmaceutically relevant natural products. These in vivo systems link the inhibition of a heterologously expressed target enzyme to a biochemical output (e.g., growth, color formation, or fluorescence); they have several important advantages over in vitro assays: (i) they can screen DNA-encoded pathways, where library size is limited by transformation efficiency; (ii) they require only a small amount of target protein, which is maintained by a living cell, and can avoid the laborious protein purification and stabilization steps required for in vitro assays; (iii) they are designed to detect inhibitors within the cellular milieu and can thus provide an initial—if, largely, general—screen for inhibitor stability and toxicity; and (iv) they facilitate rapid scale-up of molecular synthesis via microbial fermentation.

Genetically encoded biosensors for enzyme inhibitors are sparse; to date, most have focused on controlling cell viability. Illustrative strategies for protease inhibitors include (i) the addition of protease recognition sites to antibiotic resistance proteins (e.g., the metal-tetracycline/H+ antiporter) or essential regulatory enzymes (e.g., adenylate cyclase, which synthesizes cyclic AMP), or (ii) the use of proteolyzable “pro” domains to cage toxic proteins (e.g., ribosomal protein S12, which restores the streptomycin sensitivity of streptomycin-resistant E. coli). Several of these systems have enabled the detection of peptide inhibitors synthesized in microbial hosts, but their direct modification of phenotype-specific proteins (e.g., the adenylate cyclase) tends to limit their rapid extension to other proteases or biochemical outputs.

Also provided, in some aspects, are modified synthase enzymes (e.g., terpene synthases) expressed by the genetically-encoded systems disclosed herein. In some embodiments, the system has been modified to increase the diversity of the modulators produced by the cell. In some embodiments, the nucleic acid molecules encoding the synthase (e.g., enzyme responsible for producing the therapeutic target, e.g., protease or a phosphatase) may be modified to produce mutant synthase enzymes in the cell that produce a more diverse range of therapeutic targets against which the cell produces a more diverse range of modulators. For example, as described herein, the synthase responsible for producing terpenoids (e.g., terpene synthase) may be modified to produce a wider range of terpenes or terpenoid. In some embodiments, γ-humulene synthase, a low-producing terpene synthase generating many products, is mutated at one or more (e.g., 2) amino acid positions under conditions sufficient produce a larger number of diverse terpenoid inhibitors. In some embodiments, the synthase variants produced at least two potential terpenoid inhibitors with titers increased 12- and 50-fold compared to the starting enzyme.

Also provided herein, in some aspects, are extensions of the system described here to screen large numbers of pathways and target enzymes. In some embodiments, molecular barcodes may be applied to one or more components of the genetically-encoded systems, such as the synthase, the metabolic pathway, the target enzyme, or any combination thereof. In some embodiments, the efficiency of the system is increased by pooling cells having barcoded components and analyzing them using multiplex sequencing analysis. Secondary sequence data analysis utilizing suitable computer programs demultiplexes the cells, and assigns the unique molecular barcode to the one or more components of the genetically-encoded systems.

In a pilot experiment described herein, the inventors of the instant disclosure combined (i) three isoprenoid pathways, (ii) 37 terpenoid pathways, and five protein tyrosine phosphatase (PTP)-specific B2H systems in a single screen. To overcome the challenge of screening with the drop-based plating the 555 possible combinations of these three sets of plasmids, barcoding both (i) the terpenoid pathways and (ii) the B2H systems were performed to reduce the required number of transformations to 15 (e.g., one for each precursor-B2H combination). In this pilot experiment, each transformation was plated on both selective and non-selective media, the pools were amplified from each plate with a PCR reaction that introduced a second barcode for the PTP of interest, and next generation sequencing was used to measure the enrichment of specific pathways. As disclosed herein, high quality, statistically significant data was obtained for a 10⁴-10⁵ variants when sequencing short amplicons, illustrating that the proposed strategy is compatible with very large biosynthetic libraries and/or numerous target two-hybrid systems. Without being bound by any particular theory, the high throughput extensions of the systems disclosed herein are applicable to systems configured to identify bioactive molecules that modulate any target enzyme, not just phosphatases as illustrated in this pilot study.

Also provided, in some aspects, are kits comprising the systems disclosed herein, and instructions for how to use the systems disclosed herein to identify novel modulators of an intended therapeutic target, or purify novel modulators of an intended therapeutic target, or a combination thereof. Such kits may comprise a container to store the system components and instructions.

### I. SYSTEMS

Provided herein are systems for identifying a novel modulator of a target enzyme, or identifying one or more metabolic pathways that produce bioactive molecules that modulate the activity of a target enzyme, or both. In some embodiments, the target enzyme is a therapeutic target (e.g., phosphatase, protease) disclosed herein. In some embodiments, systems comprise genetically encoded systems that, when introduced into a cell under suitable conditions, induces the cell to produce novel modulators of the target enzyme. The systems disclosed herein comprise the cell, which, in some cases, is referred to herein as a genetically-encoded microorganism, once it has been engineered to contain the genetically-encoded systems disclosed herein. Also provided are systems for expanding screens for the novel modulators of the target enzyme or metabolic pathways from the genetically-encoded systems using high throughput analysis, such as multiplex sequencing. To that end, certain computer systems are also encompassed in the systems disclosed herein, which store and are programmed to perform instructions for analyzing the multiplex sequencing results, such as demultiplexing, sequence alignment, and so forth.

### A. Genetically-Encoded Systems

Provided herein, in some aspects are genetically-encoded systems that comprise one or more system components, such as one or more nucleic acid molecules encoding a two-hybrid system, a metabolic pathway, an enzyme for producing the target enzyme, or any combination thereof. In some embodiments, the target enzyme comprises a protease. In some embodiments, the target enzyme comprises a phosphatase (e.g., tyrosine phosphatase). In some embodiments, the two-hybrid system comprises or is a bacterial two-hybrid system. In some embodiments, the enzyme for producing the target enzyme comprises a terpene synthase. In some embodiments, the metabolic pathway comprises a mevalonate pathway, a methylerthritol 4-phosphate pathway, or a combination thereof. In some embodiments, the one or more nucleic acid molecules encoding the metabolic pathway comprises one or more metabolic intermediates for terpene synthesis. In some embodiments, the system comprise a cell. In some embodiments, the cell comprises the one or more nucleic acid molecules encoding a two-hybrid system, a metabolic pathway, an enzyme for producing the target enzyme, or any combination thereof. In some embodiments, the cell is configured to express the gene expression products from the system to facilitate production of novel modulators of an intended target enzyme by the cell.

### 1. Cells

Provided herein are cells that may be engineered to contain or express one or more systems disclosed herein. In some embodiments, the cell comprises the two-hybrid system. In some embodiments, the cell comprises the metabolic pathway. In some embodiments, the cell comprises the enzyme for producing the target enzyme (e.g., therapeutic target). In some embodiments, the enzyme is a synthase (e.g., terpene synthase). In some embodiments, the cell comprises one or more nucleic acid molecules encoding two-hybrid system, the metabolic pathway, the enzyme for producing the target enzyme, or any combination thereof.

In some embodiments, the cell comprises a microbial cell. In some embodiments, the microbial cell comprises an Escherichia coli cell. In some embodiments, the microbial cell comprises a Bacillus subtilis cell. In some embodiments, the microbial cell comprises a Cupriavidus necator cell. In some embodiments, the microbial cell comprises a Streptomyces lividans cell. In some embodiments, the microbial cell comprises a Streptomyces reveromyceticus cell. In some embodiments, the microbial cell comprises a Streptomyces venezuelae cell. In some embodiments, the microbial cell comprises a Synechococcus leopoliencsis cell. In some embodiments, the microbial cell comprises a Saccharomyces cerevisiae cell. In some embodiments, the microbial cell comprises a Saccharomyces coelicolor cell. In some embodiments, the microbial cell comprises a Pichia pastoris cell. In some embodiments, the microbial cell comprises a Pichia guilliermondii cell. In some embodiments, the microbial cell comprises a Yarrowia lipolytica cell. In some embodiments, the microbial cell comprises a Rhodosporidium toruloides cell. In some embodiments, the microbial cell comprises a Metarhizium brunneum cell. In some embodiments, the microbial cell comprises a Aspergillus niger cell. In some embodiments, the microbial cell comprises a Rhizopus oryzae cell.

In some embodiments, the cell comprises a mammalian cell. In some embodiments, the mammalian cell comprises a Chinese hamster ovary cell. In some embodiments, the mammalian cell comprises a baby hamster kidney cell. In some embodiments, the mammalian cell comprises a HeLa cell (a cervical cancer cell derived from Henrietta Lacks). In some embodiments, the mammalian cell comprises a human embryonic kidney cell. In some embodiments, the mammalian cell comprises a human retinal cell. In some embodiments, the mammalian cell comprises a Sp2/0 mouse myeloma cell. In some embodiments, the mammalian cell comprises a NS0 mouse myeloma cell.

In some embodiments, the cell is wild-type. In some embodiments, the cell is modified relative to a wild-type cell of the same type. For example, the cell may be modified to express the metabolic pathway prior to introducing the two-hybrid system into the cell. In another example, the cell may be modified to express the two-hybrid system prior to introducing the metabolic pathway into the cell. In another example, the cell may lack one or more endogenous genes, such as for example, a gene to encode the target enzyme where applicable. In another example, the cell may lack a gene for a subunit of RNA polymerase or portions thereof, such as the omega subunit. In another example, the cell may lack one or more native genes that enhance the intracellular production or intracellular accumulation of a bioactive molecule that modulates the activity of a target enzyme in the cell. In another example, the cell may have a deletion or mutation that reduces homologous recombination events likely to disrupt plasmids, such as a deletion of the recA1 gene. In some cases, the cell may have a deletion or mutation that improves the titratability of certain inducible promoters such as an arabinose-inducible promoter. In some embodiments, the cell is a cell line. In some embodiments, the cell line is immortalized.

In some embodiments, the cell is stored in a medium, such as Luria-Bertani liquid medium, Luria-Bertani solid medium, terrific broth liquid medium, terrific broth solid medium, yeast extract peptone dextrose liquid medium, yeast extract peptone dextrose solid medium, yeast synthetic drop-out medium, yeast nitrogen base, modified minimum essential medium, Dulbecco's modified Eagle medium, Ham's F10 medium, Ham's F12 medium, Roswell Park Memorial Institute medium, Glasgow's modified minimum essential medium, or Leibovitz L-15 medium. In some embodiments, the cell is stored in a medium as a suspension or attached to a surface (e.g., flask, plate, or well). In some embodiments, the media comprises one or more media components, such as an energy source (e.g., glucose), protein, vitamins, inorganic salts, serum, growth factors, hormones, attachment factors, amino acids, peptone, carbohydrates, minerals, pH buffer system, pH indicators, metals, blood, gelling agents (e.g., agar or pectin), or any combination thereof. In some embodiments, the media is selection media that contains a means for selecting only the cells that produced a modulator of a target enzyme (e.g., terpenoid inhibitor, protease inhibitor). In some embodiments, such selection media may contain an antibiotic, antiseptic, peptone, carbohydrate, inorganic salt, chemical substances (e.g., bile salts, lithium chloride, irgasan, tamoxifen, or potassium tellurite), adenosine deaminase, cytosine deaminase, dihydrofolate reductase, dye, phage, or any combination thereof. In some embodiments, such selection media may lack an amino acid, nutrient, carbohydrate, nucleoside, inorganic salt, serum, growth factor, or any combination thereof. In some embodiments, the antibiotic comprises penicillin, streptomycin, ampicillin, carbenicillin, spectinomycin, bleomycin, novobiocin, doxycycline, tetracycline, neomycin, kanamycin, zeocin, puromycin, geneticin, amphotericin, gentamicin, polymyxin B, hygromycin B, blasticidin, vancomycin, erythromycin, chloramphenicol, ticarcillin, or cefixime. In some embodiments, the media is a growth cell medium. In some embodiments, the growth cell medium may comprise glycerol at a concentration between 0% and 2% (by volume). In some embodiments, the growth medium comprises mevalonate at a concentration between 0 mM and 20 mM. In some embodiments, the growth medium comprises isopropyl β-D-thiogalactopyranoside (iPTG) at a concentration between 0 mM and 0.5 mM. In some embodiments, the growth medium comprises 3-morpholinopropane-1-sulfonic acid (MOPS) at a concentration between 0 mM and 50 mM. In some embodiments, the growth medium comprises sucrose at a concentration between 0% and 5% weight/volume.

The cells disclosed here may be isolated or purified. Suitable methods of purifying or isolating a cell may be found in Invitrogen, Gibco. “Cell culture basics.” Life technologies (2014), Sivashanmugam, Arun, et al. “Practical protocols for production of very high yields of recombinant proteins using Escherichia coli.” Protein science 18.5 (2009): 936-948, and Clontech. “Yeast Protocols Handbook.” Takara Bio (2009), each of which is incorporated by reference in its entirety.

In some embodiments, the cell comprises a prokaryotic cell. In some embodiments, the cell is obtained from a unicellular organism. In some embodiments, the cell is or comprises a bacterial cell, an algae cell, an archaea cell, a protozoa cell, or a fungal cell. In some embodiments, the fungal cell may be a yeast cell. In some embodiments, the bacterial cell may be an E. coli cell. In some embodiments, the cell is isolated or purified. In some embodiments, the cell is in a cell line or cell culture. In some embodiments, a plurality of cells are provided, wherein each cell comprises a unique expression system disclosed herein.

### 2. Two-Hybrid Systems

Provided herein are improved systems for producing novel modulators of a target enzyme (e.g., therapeutic target) by linking expression of a gene of interest (GOI) with production of a novel modulator with a two-hybrid system. In some embodiments, the two-hybrid system comprises a bacterial two-hybrid (B2H) system. In some embodiments, the two-hybrid system comprises a yeast two-hybrid (Y2H) system. In some embodiments, the two-hybrid system is a fluorescent two-hybrid system. In some embodiments, the two-hybrid system is an enzymatic two-hybrid system. In some embodiments, the Y2H is a slit-ubiquitin Y2H system. In some embodiments, the GOI encodes a survival advantage (e.g., antibacterial resistance) for the cell such that the two-hybrid system utilizes cell survival as a selection pressure, to identify cells that produced the modulators of the target enzyme.

In some embodiments, the two-hybrid system comprises one or more nucleic acid molecules encoding a receptor (e.g. phosphorylated protein binding domain), a DNA binding protein (e.g., repressor element), a subunit of RNA polymerase or portions thereof, a ligand (e.g. kinase substrate), a target enzyme, an operator for the repressor element, or a combination thereof. In some embodiments, where the receptor is or comprises a phosphorylated protein binding domain and the ligand is or comprises a kinase substrate, then the one or more nucleic acid molecules also encode a kinase. In some embodiments, the one or more nucleic acid molecules comprises a binding site for the subunit for the RNA polymerase configured to bind to the subunit for RNA polymerase and initiate transcription of a gene of interest (GOI), such as a reporter gene. In some embodiments, the phosphorylated protein binding domain is a phosphorylated tyrosine binding domain. In some embodiments, the kinase substrate is a tyrosine kinase substrate. In some embodiments, the kinase is a tyrosine kinase. In some embodiments the GOI is a reporter gene. In some embodiments, the one or more nucleic acid molecules further encodes a chaperone polypeptide. In some embodiments, the one or more nucleic acid molecules is or comprises an expression vector. In some embodiments, the expression vector is or comprises a plasmid vector, a viral vector, a cosmid, an artificial chromosome, or a region of the host chromosome. In some embodiments, the two-hybrid system comprises less than or equal to 1, 2, 3, 4, 5, 6, 7, 8, 9, or 10 nucleic acid molecules encoding the two-hybrid system. In some embodiments, more than or equal to 1, 2, 3, 4, 5, 6, 7, 8, 9, or 10 nucleic acid molecules encode the two-hybrid system. In some embodiments, the two-hybrid system comprises two (2) nucleic acid molecules encoding the two-hybrid system. In the case of a two-hybrid system comprising or consisting of 2 nucleic acid molecules, in some embodiments, the first nucleic acid molecule encodes the receptor (e.g., phosphorylated tyrosine binding domain), a repressor element, a subunit of RNA polymerase or portions thereof, ligand (e.g., a tyrosine kinase substrate), tyrosine kinase, and the target enzyme; and the second nucleic acid molecule encodes the operator for the repressor element and comprises a binding site for the subunit for the RNA polymerase.

In some embodiments, the receptor comprises a polypeptide suitable for binding the ligand. In some embodiments, the receptor is or comprises a ligand-binding domain. In some embodiments, the receptor is or comprises an antibody, single-domain antibody, single-chain fragment (scFv), miniprotein, a phosphorylated protein binding protein or domain thereof, or a ligand-binding portion thereof. In some embodiments, the receptor and ligand binding (e.g., forming a receptor-ligand pair) is phosphorylation dependent. For example, the receptor is or comprises a phosphorylated protein binding domain and the ligand is or comprises a kinase substrate, such that when the kinase substrate is phosphorylated, it binds to the receptor. In some embodiments, the phosphorylated protein binding domain comprises a phosphorylated serine/threonine binding domain. In some embodiments the phosphorylated serine/threonine binding domain comprises a 14-3-3, polo box, FHA, FF, BRCT, WW, WD40, or MH2 domain. In some embodiments, the phosphorylated protein binding domain comprises or is a phosphorylated tyrosine binding domain. In some embodiments, the phosphorylated tyrosine binding domain comprises Src homology 2 (SH2) domain, a phosphotyrosine-binding domain (PTB), or phosphotyrosine-interaction (PI) domain. In some embodiments, the phosphorylated protein binding domain comprises a modified or truncated polypeptide. In some embodiments, the phosphorylated protein binding domain comprises a truncated SH2. In some embodiments, the receptor and ligand binding is not phosphorylation dependent. In some embodiments, the receptor is or comprises an antibody or antigen-binding fragment thereof. In some embodiments, the ligand comprises a monobody, such as the HA4 monobody. In some embodiments, the receptor comprises an SH2 domain that can bind to nonphosphorylated proteins. In some embodiments, the receptor comprises the SH2 domain from Abl kinase. In some embodiments the ligand comprises an SspA binding domain. In some embodiments, the SspA binding domain is coupled to a light oxygen voltage 2 (LOV2) domain from Avena sativa such that it is partially obscured when LOV2 is in its dark state. In some embodiments, the receptor comprises a SspB domain, which is capable of binding to the SspA domain.

In some embodiments, the DNA binding protein is suitable for binding to a transcriptional start site of a gene of interest disclosed here. In some embodiments, the DNA binding protein is or comprises a repressor element. In some embodiments, the repressor element functions to repress transcription of the gene of interest. In other embodiments, the repressor element does not function to repress transcription of the gene of interest. In such embodiments, virtually any DNA binding protein will work in the two-hybrid system disclosed herein. Non-limiting DNA binding proteins include enhancers, transcription factors, or repressors. In some embodiments, the repressor element comprises a cI repressor. In some embodiments, the repressor element is a CymR repressor. In some embodiments, the repressor element is a Cro repressor. In some embodiments, the repressor element is any protein that binds to DNA with an affinity sufficient to activate transcription of a nearby gene of interest when the repressor element is fused to a subunit of RNA polymerase or portions thereof such that it can localize RNA polymerase to the gene of interest. In some embodiments, the repressor element is a nuclease DNA binding element. In some embodiments, the repressor element is a Cas DNA binding element. In some embodiments, the repressor element is a transcription factor.

In some embodiments, the subunit of the RNA polymerase is derived from a prokaryotic organism. In some embodiments, the prokaryotic organism is a microbe, such as bacteria, archaea, protozoa, fungi, algae, lichens, slime molds, viruses, or prions. In some embodiments, the bacteria comprises Escherichia Coli, Bacillus Subtilis, Mycobacterium, Streptomyces, or Cyanobacteria. In some embodiments, the bacteria comprises E. Coli. In some embodiments, the subunit of the RNA polymerase is derived from a eukaryotic organism. In some embodiments, the eukaryotic organism is Arabidopsis thaliana, yeast, fly (e.g., Drosophila melanogaster), worm (e.g., Caenorhabditis elegans), zebrafish (e.g., Danio reiro), or mouse (e.g., Mus musculus). In some embodiments, the subunit of the RNA polymerase or portions thereof comprises an omega subunit of RNA polymerase (RPω, encoded by gene RpoZ). In some embodiments, RPω (RpoZ) may be identified with National Library of Medicine (NCBI) Gene ID: 12930353). In some embodiments, the subunit of RNA polymerase or portions thereof comprises an alpha subunit of RNA polymerase (RPα, encoded by gene rpoA). In some embodiments, the subunit or portions thereof is a sigma factor. In the case of eukaryotic RNA polymerase, in some embodiments, the RNA polymerase is or comprises RNA polymerase II. A portion of a subunit of an RNA polymerase disclosed herein may be, for example, the portion of the subunit that recruiting RNA polymerase to the transcriptional start site of a GOI disclosed herein. In some embodiments, the portion of the subunit of RNA polymerase comprises the N-terminus of the amino acid sequence of the subunit, the C-terminus of the amino acid sequence of the subunit, both the N-terminus and the C-terminus of the amino acid sequence of the subunit, or neither of the N-terminus and the C-terminus of the amino acid sequence of the subunit.

In some embodiments, the kinase comprises a serine/threonine kinase. In some embodiments, the kinase comprises or is a tyrosine kinase. In some embodiments, the tyrosine kinase comprises Src Kinase. In some embodiments, Src Kinase is derived from Homo sapiens (human), which may be identified with NCBI Gene ID: 6714. In some embodiments, the Src Kinase is derived from Mus musculus (Mouse), Gallus gallus (Chicken), Rattus norvegicus (Rat), or Bos taurus (Bovine). In some embodiments, Src Kinase comprises an amino acid sequence comprising SEQ ID NO 74. In some embodiments, Src Kinase comprises an amino acid sequence that is greater than or equal to 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 74. In some embodiments, the kinase is or comprises isopentenyl kinase. In some embodiments, isopentenyl kinase comprises an amino acid sequence provided in SEQ ID NO: 269. In some embodiments isopentenyl kinase comprises an amino acid sequence that is greater than or equal to 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 269. In some embodiments, the kinase is or comprises Choline kinase. In some embodiments, Choline kinase comprises an amino acid sequence provided in SEQ ID NO: 267. In some embodiments Choline kinase comprises an amino acid sequence that is greater than or equal to 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 267. In some embodiments, the kinase is a portion of a kinase enzyme, such as a truncated version of any one of SEQ ID NOS: 74, 269, or 267. In some embodiments, the truncation comprises a truncation of an N-terminus, a C-terminus, or both of the amino acid sequence. In some embodiments, the Src kinase comprises a truncation of amino acids 1-250, such as in SEQ ID NO: 246. In some embodiments, the Lck kinase comprises a truncation of amino acids 1-206 and 497-509, such as in SEQ ID NO: 247. In some embodiments, the kinase is or comprises lymphocyte-specific protein tyrosine kinase (Lck). In some embodiments, the kinase is or comprises Fyn kinase. In some embodiments, Fyn kinase comprises an amino acid sequence provided in SEQ ID NO: 248. In some embodiments, Fyn kinase comprises an amino acid sequence that is greater than or equal to 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 248. In some embodiments, the kinase is or comprises proto-oncogene tyrosine-protein kinase (Yes). In some embodiments, Yes kinase comprises an amino acid sequence provided in SEQ ID NO: 249. In some embodiments, Yes kinase comprises an amino acid sequence that is greater than or equal to 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 249. In some embodiments, the kinase is or comprises tyrosine kinase EphA2 (EphA2). In some embodiments, EphA2 comprises an amino acid sequence provided in SEQ ID NO: 250. In some embodiments, EphA2 comprises an amino acid sequence that is greater than or equal to 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 250. In some embodiments, the kinase is or comprises Bruton's tyrosine kinase (BTK). In some embodiments, BTK comprises an amino acid sequence provided in SEQ ID NO: 251. In some embodiments, BTK comprises an amino acid sequence that is greater than or equal to 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 251

In some embodiments, the chaperone polypeptide comprises Hsp90 co-chaperone Cdc37. In some embodiments, the chaperone polypeptide comprises the GroEL/GroES complex. In some embodiments, Cdc37 comprises an amino acid sequence comprising SEQ ID NO 76. In some embodiments, Cdc37 comprises an amino acid sequence that is greater than or equal to 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 76.

In some embodiments, the components above (e.g., kinase, chaperone, receptor, ligand, etc.) may be derived from a prokaryotic organism. In some embodiments, the prokaryotic organism is a microbe, such as bacteria, archaea, protozoa, fungi, algae, lichens, slime molds, viruses, or prions. In some embodiments, the bacteria comprises Escherichia Coli, Bacillus Subtilis, Mycobacterium, Streptomyces, or Cyanobacteria. In some embodiments, the bacteria comprises E. Coli. In some embodiments, the components above may be derived from a eukaryotic organism. In some embodiments, the eukaryotic organism is Arabidopsis thaliana, yeast, fly (e.g., Drosophila melanogaster), worm (e.g., Caenorhabditis elegans), zebrafish (e.g., Danio reiro), or mice (e.g., Mus musculus).

Two or more two-hybrid system components may be coupled to each other. In some embodiments two or more of the receptor (e.g., phosphorylated tyrosine binding domain), the DNA binding protein (e.g., repressor element), the subunit of RNA polymerase or portions thereof, the ligand (e.g., tyrosine kinase substrate), the tyrosine kinase, the target enzyme, the operator for the repressor element, are coupled to each other. In some embodiments, the receptor (e.g., phosphorylated tyrosine binding domain) is coupled to the DNA binding protein (e.g., repressor element). In some embodiments, the SH2 domain is coupled with the cI repressor. In some embodiments, the subunit of the RNA polymerase or portions thereof is coupled with the ligand (e.g., tyrosine phosphatase substrate). In some embodiments, the RpoZ is coupled to the ligand (e.g., tyrosine phosphatase substrate). In some embodiments, the receptor (e.g., phosphorylated tyrosine binding domain) is coupled to the ligand (e.g., tyrosine phosphatase substrate). In some embodiments, the SH2 domain is coupled to the tyrosine phosphatase substrate. In some embodiments, the repressor element is coupled to the subunit of the RNA polymerase or portions thereof. In some embodiments, the cI repressor is coupled to the RpoZ. In some embodiments, the two or more components of the two-hybrid system are coupled to each other by fusion (e.g., expression of a fusion protein). In some embodiments, the two or more components of the two-hybrid system are coupled to each other with a linker. In some embodiments, the linker comprises a chemical linker, a peptide linker, or both. In some embodiments, the peptide linker is an alanine linker. In some embodiments, the linker binds components through peptide bonds, covalent bonds, ionic bonds, hydrogen bonds, disulfide bonds, or hydrophilic or hydrophobic interactions. Non-limiting examples of peptide linkers can be found here Chen, Xiaoying, Jennica L. Zaro, and Wei-Chiang Shen. “Fusion protein linkers: property, design and functionality.” Advanced drug delivery reviews 65.10 (2013): 1357-1369, which is hereby incorporated by reference in its entirety.

In some embodiments, the RNA polymerase binding site is suitable for binding with an RNA polymerase disclosed herein. In some embodiments, the subunit of RNA polymerase or portions thereof encoded by the genetically-encoded system disclosed herein recruits RNA polymerase to the RNA polymerase binding site to initiate transcription of a gene of interest. In such embodiments, the RNA polymerase binding site may be in a transcriptional activation site or region of the gene of interest. In some embodiments, the binding site for the RNA polymerase is a binding site for the subunit of the RNA polymerase or portions thereof. In some embodiments, a sigma factor enables binding of RNA polymerase to a gene promoter.

In some embodiments, the gene of interest (GOI) is a reporter gene that encodes a reporter polypeptide. In some embodiments, the reporter polypeptide comprises a luciferase enzyme, a fluorescent polypeptide, alkaline phosphatase, ß-galactosidase, a fructosyltransferase (e.g., levansucrase), chloramphenicol acetyltransferase (CAT), or a polypeptide that confers resistance to an antibiotic. In some embodiments, the antibiotic is penicillin, streptomycin, ampicillin, carbenicillin, spectinomycin, bleomycin, novobiocin, doxycycline, tetracycline, neomycin, kanamycin, zeocin, puromycin, geneticin, amphotericin, gentamicin, polymyxin B, hygromycin B, blasticidin, vancomycin, erythromycin, chloramphenicol, ticarcillin, or cefixime. Non-limiting examples of reporter genes encoding resistance to an antibiotic include, beta-lactamases, bleomycin binding protein Ble-MBL, blasticidin S deaminase, aminoglycoside adenylyltransferase, aminoglycoside phosphotransferase, tetracycline efflux protein, puromycin N-acetyltransferase, chloramphenicol acetyltransferase, neomycin phosphotransferase II, sterol 24-C-methyltransferase, bifunctional enzyme AAC/APH, or mobilized colistin resistance. Non-limiting fluorescent polypeptides include, but are not limited to green fluorescent protein, enhanced green fluorescent protein, green fluorescent protein ultra violet, blue fluorescent protein, enhanced blue fluorescent protein yellow fluorescent protein, enhanced yellow fluorescent protein, red fluorescent protein, DsRed fluorescent protein, cyan fluorescent protein, enhanced cyan fluorescent protein, mCherry, m Turquoise, m Venus, mRuby mWasabi, mTagBFP, mCitrine, mBanana, mOrange, dTomato, and Emerald.

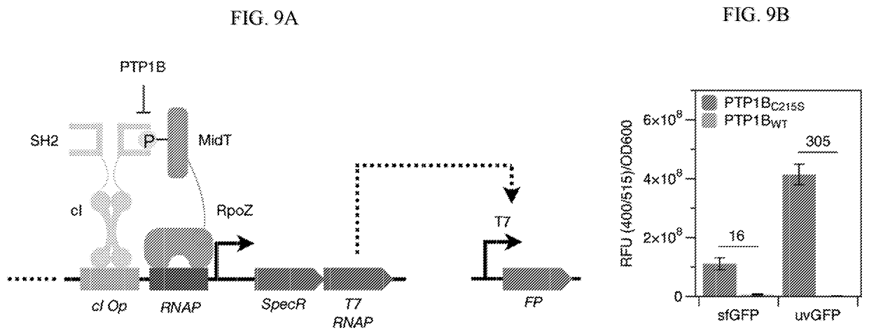

In some embodiments, the GOI encodes a polymerizing enzyme or transcriptional activator that, when expressed, binds to a promoter or enhancer operably linked to a gene encoding a reporter polypeptide to drive expression of the reporter polypeptide disclosed herein. In some embodiments, the GOI encodes a polymerizing enzyme or repressor that, when expressed, binds to a promoter or transcriptional start site operably linked to a gene encoding the reporter polypeptide to reduce expression of the reporter polypeptide disclosed herein. In either case, the variant expression of the reporter polypeptide (e.g., increased expression in the case of the polymerizing enzyme or activator; decreased expression in the case of the polymerizing enzyme or repressor) as compared to a reference expression of the reporter polypeptide may be a readout of the genetically-encoded systems disclosed herein. In some embodiments, the reporter polypeptide is a detectable polypeptide. In some embodiments, a detectable polypeptide comprises a fluorescent polypeptide, such as those disclosed herein. In some embodiments, the polymerizing enzyme comprises an RNA polymerase. In some embodiments, the RNA polymerase comprises a prokaryotic RNA polymerase. In some embodiments, the RNA polymerase comprises a eukaryotic RNA polymerase. In some embodiments, the RNA polymerase is derived from a virus or bacteriophage. In some embodiments, the RNA polymerase comprises T7 RNA Polymerase (T7 RNAP), SP6 RNA Polymerase, or T3 RNA Polymerase. In some embodiments, the prokaryotic RNA polymerase is derived from a bacterium, archaea, or algae. In some embodiments, the RNA polymerase comprises Escherichia coli RNA Polymerase, Escherichia coli RNA Polymerase core enzyme, Escherichia coli RNA Polymerase holoenzyme, Poly(A) Polymerase, or plastid-encoded RNA polymerase. In some embodiments, the eukaryotic RNA polymerase is derived from a yeast, mammal, or plant. In some embodiments, the eukaryotic RNA polymerase comprises RNA polymerase I, RNA polymerase II, RNA polymerase III, RNA polymerase IV, RNA polymerase V, or chloroplast-derived plastid-encoded polymerase. In some embodiments, the RNA polymerase is a modified version of the wild-type RNA polymerase. In some embodiments, the RNA polymerase comprises one or more mutations of an amino acid sequence to improve fidelity, affinity, or both. In some embodiments, a subunit of the RNA polymerase or portions thereof sufficient to induce expression of the gene of interest is used rather than the entire RNA polymerase. In some embodiments, when the GOI encodes a polymerizing enzyme or a transcriptional activator that induces expression (e.g., activates transcription) of a reporter polypeptide that is detectable, the detectable signal or readout from the detectable polypeptide is greater than if the GOI encoded the detectable polypeptide. In some embodiments, the signal or readout is greater than by about 1-fold, 2-fold, 3-fold, 4-fold, 5-fold, 6-fold, 7-fold, 8-fold, 9-fold, or 10-fold. In some embodiments, the signal or readout from the detectable polypeptide is greater than by about 50%, 55%, 60%, 65%, 70%, 75%, 80%, 85%, 90%, 95%, or 100%. In some embodiments, the signal or readout from the detectable polypeptide comprises from 1-fold to 10-fold, from 2-fold to 9-fold, from 3-fold to 8-fold, from 4-fold to 7-fold, or from 5-fold to 6-fold greater. In some embodiments, the signal or readout from the detectable polypeptide comprises from 50% to 100%, from 55% to 95%, from 60% to 90%, from 65% to 85%, or from 70% to 80% greater. In some embodiments, the extent of signal amplification cannot be quantified because the detectable polypeptide yields no detectable signal when included as the GOI, rather than as a gene regulated by an activator or polymerizing enzyme encoded by the GOI. As an example, the reporter gene may encode T7 RNA Polymerase (T7 RNAP), that when expressed in the presence of an inhibitor of the target enzyme, drives expression of a fluorescent protein (FP), as shown in FIG. 9. In such an example, expression of the fluorescent protein (e.g., green fluorescent protein, GFP) is over 4-fold greater than if GFP were encoded by the reporting gene in the two-hybrid system. In another example, expression of the fluorescent protein (e.g., green fluorescent protein, GFP) is over 2-fold greater than if GFP were encoded by the reporting gene in the two-hybrid system. In another example, expression of the fluorescent protein (e.g., green fluorescent protein, GFP) is over 3-fold greater than if GFP were encoded by the reporting gene in the two-hybrid system. In another example, expression of the fluorescent protein (e.g., green fluorescent protein, GFP) is over 1-fold greater than if GFP were encoded by the reporting gene in the two-hybrid system. In another example, expression of the fluorescent protein (e.g., green fluorescent protein, GFP) is over 5-fold greater than if GFP were encoded by the reporting gene in the two-hybrid system. In some embodiments, when the GOI encodes a polymerizing enzyme or a transcriptional repressor that induces expression (e.g., represses transcription) of a reporter polypeptide that is detectable, the difference in detectable signal or readout from the detectable polypeptide is greater than if the GOI encoded the detectable polypeptide. In some embodiments, the signal or readout is less than by about 1-fold, 2-fold, 3-fold, 4-fold, 5-fold, 6-fold, 7-fold, 8-fold, 9-fold, or 10-fold. In some embodiments, the signal or readout from the detectable polypeptide is less than by about 50%, 55%, 60%, 65%, 70%, 75%, 80%, 85%, 90%, 95%, or 100%. In some embodiments, the signal or readout from the detectable polypeptide comprises from 1-fold to 10-fold, from 2-fold to 9-fold, from 3-fold to 8-fold, from 4-fold to 7-fold, or from 5-fold to 6-fold less. In some embodiments, the signal or readout from the detectable polypeptide comprises from 50% to 100%, from 55% to 95%, from 60% to 90%, from 65% to 85%, or from 70% to 80% less. In some embodiments, the extent of signal amplification cannot be quantified because the detectable polypeptide yields no detectable signal when included as the GOI, rather than as a gene regulated by the repressor or polymerizing enzyme encoded by the GOI.

a. Target Enzymes

Disclosed herein are target enzymes. In some embodiments, the target enzymes disclosed herein are therapeutic targets. In some embodiments, the target enzymes are encoded by the two-hybrid systems described herein. In some embodiments, the target enzyme may be associated with, or cause, a disease or a condition disclosed herein, such as cancer. In some embodiments, the target enzyme may be associated with, or cause, an infection or a disease or a condition associated with an infection by a pathogen. In some embodiments, the pathogen may be a virus, a bacterium, a fungus, a parasite, or a prion. In some embodiments, the target enzyme may be an enzyme that is expressed by one or more cancer cells.

Non-limiting examples of diseases or conditions that are associated with, or caused by, an infection by a pathogen include the common cold or viral rhinitis, influenza, meningitis, herpes, warts, measles, viral gastroenteritis, toxoplasmosis, encephalitis, tuberculosis, certain types of cancer such as cervical cancer, pneumonia, sepsis, pre-term or still birth, Ebola virus disease, Zika virus disease, Coronavirus disease, Lassa fever, Crimean-Congo hemorrhagic fever, Cholera, Dengue, Hepatitis, HIV/AIDS, diarrhea, Echinococcosis, Malaria, Polio, Tetanus, Rabies, Monkeypox, or smallpox.

Non-limiting examples of diseases or conditions that are associated with, or caused by, aberrant protease activity include cancer, diabetes, cardiovascular disease, inflammation, neurological disease, atherosclerosis, thrombosis, aneurysm, pulmonary hypertension, arthritis, osteoporosis, and chronic obstructive pulmonary disease.

In some embodiments, the target enzyme comprises a wild-type sequence. In some embodiments, the target enzyme is derived from an animal (e.g., mammals, mollusks, or cnidarians), plant, bacteria, virus, bacteriophage, chromistan, protist, or fungus. In some embodiments, the mammal is a monkey, primate, or human. In some embodiments, the mammal is a human. In some embodiments, the target enzyme is modified relative to the wild-type target enzyme. In some embodiments, the modification is an insertion, a substitution, or a deletion of one or more amino acids with reference to the wild-type sequence. In some embodiments, the modification is at one or more amino acid positions of the wild-type sequence. In some embodiments, the target enzyme expressed by the genetically-encoded system comprises a truncation at an N terminus, a C terminus, or both of the amino acid sequence of the target enzyme. In some embodiments, the truncation comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids. In some embodiments, the truncation comprises fewer than or equal to about 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 amino acids. In some embodiments, the truncation comprises greater than or equal to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids. In some embodiments, the truncation comprises 1-40, 2-39, 3-38, 4-37, 5-36, 6-35, 7-34, 8-33, 9-32, 10-31, 11-30, 12-29, 13-28, 14-27, 15-26, 16-25, 17-24, 18-23, 19-22, 20-21 amino acids. Non-limiting examples of truncated target enzymes are provided in Table 28.

In some embodiments, the target enzyme comprises a phosphatase or another enzyme capable of removing a phosphate group from a substrate, such as a protein, or a catalytically active portion thereof. In some embodiments, the phosphatase is capable of dephosphorylating a histidine, isoleucine, leucine, lysine, methionine, phenylalanine, threonine, tryptophan, valine, alanine, asparagine, aspartic acid, glutamic acid, serine, arginine, cysteine, glutamine, glycine, proline, or tyrosine. In some embodiments, the phosphatase comprises or is a tyrosine phosphatase. Non-limiting examples of protein tyrosine phosphatases are provided in Tautz L, Critton D A, Grotegut S. Protein tyrosine phosphatases: structure, function, and implication in human disease. Methods Mol Biol. 2013; 1053:179-221, which is hereby incorporated by reference in its entirety. In some embodiments, the tyrosine phosphatase comprises Protein tyrosine phosphatase non-receptor type 1 (PTP1B), Protein tyrosine phosphatase non-receptor type 2 (TC-PTP), Protein tyrosine phosphatase non-receptor type 6 (SHP1), Protein tyrosine phosphatase non-receptor type 11 (SHP1), or Protein tyrosine phosphatase non-receptor type 12 (PTP-PEST). In some embodiments, the tyrosine phosphatase is a receptor tyrosine phosphatase. In some embodiments, the tyrosine phosphatase comprises a cysteine-specific protein tyrosine phosphatase. In some embodiments, the tyrosine phosphatase is derived from Homo sapiens (human). In some embodiments, human PTP1B can be identified by NCBI Gene ID: 5770. In some embodiments, human TCPTP can be identified by NCBI Gene ID: 5771. In some embodiments, human SHP1 can be identified by NCBI Gene ID: 5777. In some embodiments, human PTP-PEST can be identified by NCBI Gene ID: 5782. Non-limiting examples of tyrosine phosphatases include PTP1B (SEQ ID NOS: 6 and 236), TCPTP (SEQ ID NOS: 237-238), PTPRB (SEQ ID NO: 239), PTPRC (SEQ ID NO: 240), PTPN6 (SEQ ID NO: 241), PTPN22 (SEQ ID NO: 242), PTPRS (SEQ ID NO: 243), PTPRM (SEQ ID NO: 244), or PTPRZ (SEQ ID NO: 245). In some embodiments, the tyrosine phosphatase comprises an amino acid sequence provided in SEQ ID NO: 6. In some embodiments, the tyrosine phosphatase comprises an amino acid sequence that is greater than or equal to about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 6. In some embodiments, the tyrosine phosphatase comprises an amino acid sequence provided in SEQ ID NO: 235. In some embodiments, the tyrosine phosphatase comprises an amino acid sequence that is greater than or equal to about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 235. In some embodiments, the tyrosine phosphatase comprises an amino acid sequence provided in SEQ ID NO: 236. In some embodiments, the tyrosine phosphatase comprises an amino acid sequence that is greater than or equal to about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 236. In some embodiments, the tyrosine phosphatase comprises an amino acid sequence provided in SEQ ID NO: 237. In some embodiments, the tyrosine phosphatase comprises an amino acid sequence that is greater than or equal to about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 237. In some embodiments, the tyrosine phosphatase comprises an amino acid sequence provided in SEQ ID NO: 238. In some embodiments, the tyrosine phosphatase comprises an amino acid sequence that is greater than or equal to about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 238. In some embodiments, the tyrosine phosphatase comprises an amino acid sequence provided in SEQ ID NO: 239. In some embodiments, the tyrosine phosphatase comprises an amino acid sequence that is greater than or equal to about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 239. In some embodiments, the tyrosine phosphatase comprises an amino acid sequence provided in SEQ ID NO: 240. In some embodiments, the tyrosine phosphatase comprises an amino acid sequence that is greater than or equal to about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 240. In some embodiments, the tyrosine phosphatase comprises an amino acid sequence provided in SEQ ID NO: 241. In some embodiments, the tyrosine phosphatase comprises an amino acid sequence that is greater than or equal to about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 241. In some embodiments, the tyrosine phosphatase comprises an amino acid sequence provided in SEQ ID NO: 242. In some embodiments, the tyrosine phosphatase comprises an amino acid sequence that is greater than or equal to about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 242. In some embodiments, the tyrosine phosphatase comprises an amino acid sequence provided in SEQ ID NO: 243. In some embodiments, the tyrosine phosphatase comprises an amino acid sequence that is greater than or equal to about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 243. In some embodiments, the tyrosine phosphatase comprises an amino acid sequence provided in SEQ ID NO: 244. In some embodiments, the tyrosine phosphatase comprises an amino acid sequence that is greater than or equal to about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 244. In some embodiments, the tyrosine phosphatase comprises an amino acid sequence provided in SEQ ID NO: 245. In some embodiments, the tyrosine phosphatase comprises an amino acid sequence that is greater than or equal to about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 245.

In some embodiments, the tyrosine phosphatase is truncated. In some embodiments, the truncation is the N-terminus or the C-terminus, or both of the amino acid sequence. In some embodiments, the truncated tyrosine phosphatase is or comprise a catalytic domain of the phosphatase (e.g., a portion there cable of performing a phosphatase catalytic function). In some embodiments, the tyrosine phosphatase comprises an amino acid sequence provided in Table 27. In some embodiments, the catalytic domains of the tyrosine phosphatases described herein comprises an amino acid sequence provided in Table 28. In some embodiments, the tyrosine phosphatase comprises an amino acid sequence that is provided in any one of SEQ ID NOS: 235-245. In some embodiments, the tyrosine phosphatase comprises an amino acid sequence that is greater than or equal to about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOS: 235-245. In some embodiments, the tyrosine phosphatase comprises an amino acid sequence provided in SEQ ID NO: 235. In some embodiments, the tyrosine phosphatase comprises an amino acid sequence that is greater than or equal to about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 235. In some embodiments, the tyrosine phosphatase comprises an amino acid sequence provided in SEQ ID NO: 236. In some embodiments, the tyrosine phosphatase comprises an amino acid sequence that is greater than or equal to about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 236. In some embodiments, the tyrosine phosphatase comprises an amino acid sequence provided in SEQ ID NO: 237. In some embodiments, the tyrosine phosphatase comprises an amino acid sequence that is greater than or equal to about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 237. In some embodiments, the tyrosine phosphatase comprises an amino acid sequence provided in SEQ ID NO: 238. In some embodiments, the tyrosine phosphatase comprises an amino acid sequence that is greater than or equal to about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 238. In some embodiments, the tyrosine phosphatase comprises an amino acid sequence provided in SEQ ID NO: 239. In some embodiments, the tyrosine phosphatase comprises an amino acid sequence that is greater than or equal to about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 239. In some embodiments, the tyrosine phosphatase comprises an amino acid sequence provided in SEQ ID NO: 240. In some embodiments, the tyrosine phosphatase comprises an amino acid sequence that is greater than or equal to about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 240. In some embodiments, the tyrosine phosphatase comprises an amino acid sequence provided in SEQ ID NO: 241. In some embodiments, the tyrosine phosphatase comprises an amino acid sequence that is greater than or equal to about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 241. In some embodiments, the tyrosine phosphatase comprises an amino acid sequence provided in SEQ ID NO: 242. In some embodiments, the tyrosine phosphatase comprises an amino acid sequence that is greater than or equal to about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 242. In some embodiments, the tyrosine phosphatase comprises an amino acid sequence provided in SEQ ID NO: 243. In some embodiments, the tyrosine phosphatase comprises an amino acid sequence that is greater than or equal to about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 243. In some embodiments, the tyrosine phosphatase comprises an amino acid sequence provided in SEQ ID NO: 244. In some embodiments, the tyrosine phosphatase comprises an amino acid sequence that is greater than or equal to about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 244. In some embodiments, the tyrosine phosphatase comprises an amino acid sequence provided in SEQ ID NO: 245. In some embodiments, the tyrosine phosphatase comprises an amino acid sequence that is greater than or equal to about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 245.

In some embodiments, the phosphatase comprises or is a serine phosphatase. In some embodiments, the serine phosphatase is a threonine phosphatase. In some embodiments, the phosphatase is a serine threonine phosphatase. Non-limiting examples of serine threonine phosphatases include Phosphoprotein phosphatases, Phosphoprotein phosphatases activated by magnesium, serine/threonine protein phosphatase 5/retinal degeneration C (PP5/rdgC), protein phosphatase with EF-hand domain 2 (PPEF2), protein phosphatase 5 catalytic subunit (PPP5C), Carboxy Terminal Domain phosphatases. In some embodiments, the phosphatase comprises or is a tyrosine, serine, and threonine phosphatase. Non-limiting examples of protein tyrosine, serine, and threonine phosphatase include Lambda Protein Phosphatase.

In some embodiments, the target enzyme is a protein tyrosine phosphatase. In some embodiments, the protein tyrosine phosphatase is a nonreceptor protein tyrosine phosphatase. In some embodiments, the nonreceptor protein tyrosine phosphatase is PTP1B, PTPN2, or PTPN22. In some embodiments, the protein tyrosine phosphatase is a protein serine/threonine phosphatase. In some embodiments, the protein serine/threonine phosphatase is PP1, PP2A, or PP2B. In some embodiments, the protein tyrosine phosphatase is a dual specificity phosphatase. In some embodiments, the dual specificity phosphatase is a MAPK phosphatase, laforin, a PTEN-like phosphatase, or a Cdc14 phosphatase.

In some embodiments, the target enzyme is or comprises a proteolytic enzyme. In some embodiments, the proteolytic enzyme is a protease, peptidase or proteinase, or any other enzyme capable of hydrolyzing peptide bonds, or a catalytically active portion thereof. In some embodiments, the proteolytic enzyme hydrolyzes a peptide bond of a serine or a tyrosine. In some embodiments, the proteolytic enzyme hydrolyzes a peptide bond of a histidine, isoleucine, leucine, lysine, methionine, phenylalanine, threonine, tryptophan, valine, alanine, asparagine, aspartic acid, glutamic acid, serine, arginine, cysteine, glutamine, glycine, proline, or tyrosine. In some embodiments, the protease is derived from Homo sapiens (human) (e.g., a human protease), bacteria, archaea, algae, a virus, or a plant. In some embodiments, the protease is derived from a virus (e.g., a viral protease). In some embodiments, the human protease comprises ubiquitin specific peptidase 7 (USP7) (also referred to herein as Ubiquitin-specific-processing protease 7 (USP7)), which may be identified by NCBI Gene ID: 7874. Non-limiting examples of other human ubiquitin specific proteases include Ubiquitin-specific-processing protease 4 (USP4), Ubiquitin-specific-processing protease 11 (USP11), Ubiquitin-specific-processing protease 32 (USP32), Ubiquitin-specific-processing protease 15 (USP15), Ubiquitin-specific-processing protease 9X (USP9X), Ubiquitin carboxyl-terminal hydrolase 14 (USP14), or Ovarian tumor (OTU) domain-containing protein 7B. In some embodiments USP7 comprises an amino acid sequence comprising SEQ ID NO: 65. In some embodiments, USP7 comprises an amino acid sequence that is more than or equal to about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 65. In some embodiments, the ubiquitin specific protease comprises USP11. In some embodiments, the USP11 comprises an amino acid sequence comprising SEQ ID NOS: 288. In some embodiments, the USP11 comprises an amino acid sequence that is greater than or equal to about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NOS: 288. In some embodiments, the ubiquitin specific protease comprises USP14. In some embodiments USP14 comprises an amino acid sequence comprising SEQ ID NO: 289. In some embodiments, USP14 comprises an amino acid sequence that is greater than or equal to about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 289. In some embodiments, the ubiquitin specific protease comprises the Ovarian tumor (OTU) domain-containing protein 7B. In some embodiments, the OTU domain-containing protein 7B comprises an amino acid sequence comprising SEQ ID NO: 290. In some embodiments, the OTU domain-containing protein 7B comprises an amino acid sequence that is greater than or equal to about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 290.

In some embodiments, the protease may be 3CL protease (3CLpro), papain-like protease (PLpro), NS2B, NS3pro, NS2B-NS3pro fusion protein, 3C protease, K7L, I7L, OTU domain of L protein, NSP2. In some embodiments, the viral protease may be a protease in the family of Calciviridae, Coronaviridae, Flaviviridae, Picornaviridae, Poxviridase, Nairoviridae, or Togaviridae. In some embodiments, the viral protease comprises a protease from Norovirus GI.1, Norovirus GII.4, Severe acute respiratory syndrome (SARS), Middle East respiratory syndrome coronavirus (MERS-COV), Dengue Virus 1, Dengue Virus 2, Dengue Virus 3, Dengue Virus 4, West Nile Virus, Japanese encephalitis virus, St. Louis encephalitis virus, Yellow fever virus, Zika virus, Hepatitis A, Enterovirus 68, Enterovirus 71, Variola Major, small pox, Monkeypox virus, Crimean-Congo hemorrhagic fever orthonairovirus, Venezuelan equine encephalitis virus, Eastern equine encephalitis virus, Western equine encephalitis virus, or Chikungunya virus. In some embodiments, the viral protease is or comprises 3CLpro of severe acute respiratory syndrome coronavirus 2 (SARS-COV-2). In some embodiments 3CLpro 7 comprises an amino acid sequence comprising SEQ ID NO: 69. In some embodiments, 3CLpro comprises an amino acid sequence that is more than or equal to about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 69. In some embodiments, the viral protease is or comprises NS2B/NS3 protease of West Nile Virus. In some embodiments NS2B/NS3 protease comprises an amino acid sequence comprising SEQ ID NO: 78. In some embodiments, NS2B/NS3 protease comprises an amino acid sequence that is more than or equal to about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:78. In some embodiments, the viral protease is or comprises PLpro of SARS-COV-2. In some embodiments PLpro comprises an amino acid sequence comprising SEQ ID NO: 67. In some embodiments, PLpro comprises an amino acid sequence that is more than or equal to about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 67. In some embodiments, HIV protease (HIV-1Pr) comprises an amino acid sequence provided in SEQ ID NO: 63. In some embodiments, HIV-1Pr comprises an amino acid sequence that is more than or equal to about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 63. In some embodiments, USP7 protease comprises an amino acid sequence provided in SEQ ID NO: 65. In some embodiments, USP7 protease comprises an amino acid sequence that is more than or equal to about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 65.

In some embodiments, the target enzyme is encoded by the two-hybrid system disclosed herein. In some embodiments, the target enzyme is produced by the synthase enzyme encoded by the system disclosed herein. Certain trypsin-like serine proteases (e.g., NS3pro) may exhibit activity in the present of a cofactor (e.g., NS2B). In some embodiments, the trypsin-like serine protease and its cofactor (e.g., NS3pro and NS2B) are expressed as a protein-protein fusion or as separate proteins that forms a complex in the cell, as illustrated in FIG. 50. In some embodiments, the target enzyme may be expressed as a protein-protein fusion, a bivalent, or a polycistronic biomolecule. Bivalent or polycistronic genetic architectures, which enable independent expression of each functional component, can permit high yield expression of active protein.

In some embodiments, the target enzyme is a protein kinase. In some embodiments, the protein kinase is a protein tyrosine kinase. In some embodiments, the protein tyrosine kinase is a receptor tyrosine kinase. In some embodiments, the receptor tyrosine kinase is EGFR, HER2/ErbB2, PDGFR, FGFR, Insulin receptor, or MET. In some embodiments, the protein tyrosine kinase is a non-receptor tyrosine kinase. In some embodiments, the non-receptor tyrosine kinase is Janus kinase (JAK), focal adhesion kinase, Feline Sarcoma kinase, SYK, TEC, or Abl. In some embodiments, the protein kinase is a protein serine/threonine kinase. In some embodiments, the serine/threonine kinase is JNK, Protein Kinase B/AKT, Casein Kinase 2, Protein Kinase A, MAPKs, or mTOR, In some embodiments, the protein kinase is a Cyclin Dependent Kinase (CDK). In some embodiments, the protein kinase comprises Src Kinase, lymphocyte-specific protein tyrosine kinase (Lck), Fyn kinase Yes kinase, tyrosine kinase EphA2, or Bruton's tyrosine kinase (BTK). In some embodiments, the protein kinase is truncated. In some embodiments, the truncation is on the C-terminus, the N-terminus or a combination thereof. In some embodiments, the protein kinase comprises an amino acid sequence provided in any one of SEQ ID NOS: 246-251. In some embodiments, the protein kinase comprises an amino acid sequence that is greater than or equal to about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOS: 246-251.

b. Ligand

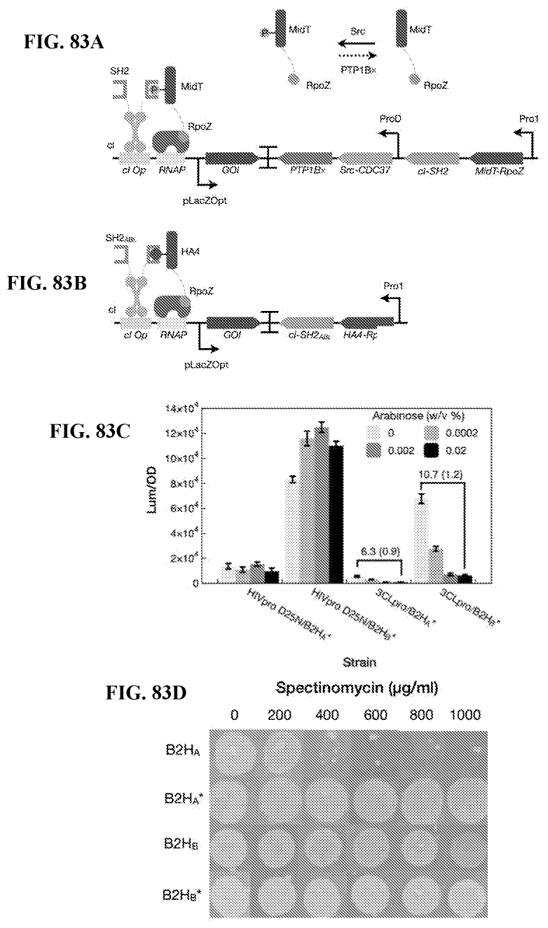

Disclosed herein are ligands capable of binding receptors encoded by the two-hybrid systems disclosed herein. In some embodiments, the ligand is a polypeptide that includes short hydrophobic peptide segments that can bind to a receptor (e.g. Hsp70, Hsp90, Per-Arnt-Sim repeats). In some embodiments, the ligand is a polypeptide with an amino acid sequence that is similar to, in part or in full, or identical to, the amino acid sequence of the receptor (e.g., homodimer cytochrome c). In some embodiments, the ligand binds to the receptor in a manner that is not phosphorylation dependent. In some embodiments, the ligand is a polypeptide that binds to the receptor through hydrogen bonds (e.g., estrogen receptor alpha/beta heterodimer). In some embodiments, the ligand interacts with the receptor through agglutination (e.g., antibody-antigen binding). In some embodiments, the ligand binds to the receptor in a manner that is phosphorylation dependent. In some embodiments, the ligand is a kinase substrate. In some embodiments, the kinase substrate may comprise a polypeptide with an amino acid residue that can be phosphorylated by a protein kinase, dephosphorylated by a protein phosphatase, bind to a phosphorylated protein binding domain (e.g., SH2 domain) in its phosphorylated state, and bind less strongly to phosphorylated protein binding domain (or not at all) when it is dephosphorylated. In some embodiments, the phosphorylated protein binding domain comprises or is a tyrosine kinase substrate. In some embodiments, the tyrosine kinase substrate may comprise a polypeptide with a tyrosine residue that can be phosphorylated by a protein tyrosine kinase, dephosphorylated by a protein tyrosine phosphatase, bind to a SH2 domain in its phosphorylated state, and bind less strongly to the SH2 domain (or not at all) when it is dephosphorylated. In some embodiments, where the binding between SH2 is not phosphorylation dependent, the kinase substrate can be SH2ABL/HA4, as shown FIG. 83B. In some embodiments, the tyrosine phosphatase substrate may comprise a substrate domain derived from the hamster polyomavirus middle T antigen (MidT).

c. Protease Cleavage Sites

Disclosed herein are protease cleavage sites that are defined by a protease recognition motif disclosed herein and configured to be cleaved by a proteolytic enzyme (e.g., a protease) disclosed herein. In some embodiments, the protease cleavage sites are engineered to in a linker region between one or more components of the two-hybrid system. In some embodiments, the protease cleavage site is located outside the linker region. In some embodiments, the two-hybrid system is the phosphorylation sensitive B2H system disclosed herein. In some embodiments, the protease cleavage site is positioned in a linker between the subunit of the RNA polymerase or portions thereof (e.g., RpoZ) and the kinase/phosphatase substrate (e.g., MidT), as shown in FIGS. 5A-5B. Referring to FIGS. 5A-5B, when the genetically encoded microorganism produces an inhibitor of the encoded protease, cleave at the cleave site does not occur, permitting recruitment of the subunit of RNA polymerase or portions thereof (e.g., RpoZ (RPω)) to bind to the RNAP binding region and inactivation of the repressor element (e.g., cI repressor) through interaction between the ligand (e.g., phosphorylated kinase/phosphatase substrate like MidT) and a phosphorylated protein binding domain (e.g., SH2) coupled to the repressor element. By contrast, in the presence of an inhibitor of the protease encoded by the system, successful cleavage of the cleavage site in the linker between the kinase/phosphatase substrate and the subunit of the RNA polymerase or portions thereof will prevent the interaction between the ligand (e.g., phosphorylated kinase/phosphatase substrate like MidT) and a phosphorylated protein binding domain (e.g., SH2) coupled to the repressor element, and thereby, prevent transcription of the reporter polypeptide (e.g., pLacZOpt).

In some embodiments, the protease recognition motif is specific to a protease disclosed herein. In some embodiments, the protease recognition motif is provided in Table 11. In some embodiments, the protease comprises HIVpro, 3CLpro of severe acute respiratory syndrome coronavirus 2 (SARS-COV-2), the papain-like protease (PLpro) of SARS-COV-2, or ubiquitin-specific-processing protease 7 (USP7). In some embodiments, these proteases are important targets for viral diseases (e.g., HIVpro, 3CLpro, and PLpro) and cancer (e.g., USP7), have protease recognition motifs that range from 4 to 75 amino acids and exhibit different yields when overexpressed in a cell (e.g., E. coli). In some embodiments, the protease is provided in Table 11.

In some embodiments, the protease recognition motifs comprise less than or equal to about 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, or 2 amino acids. In some embodiments, the recognition motifs comprise more than or equal to about 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, or 2 amino acids. In some embodiments, the recognition motifs comprise 3-100, 3-75, 3-50, 3-25, 4-100, 4-75, 4-50, 4-25, 5-100, 5-75, 5-50, 5-25, 6-100, 6-75, 6-50, 6-25, 7-100, 7-75, 7-50, 7-25, 8-100, 8-75, 8-50, 8-25, 9-100, 9-75, 9-50, 9-25, 10-100, 10-75, 10-50, or 10-25 amino acids. In some embodiments, the recognition motifs comprise 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, or 2 amino acids. In some embodiments, the amino acids are contiguous.

In some embodiments, the linker is or comprises a peptide linker. In some embodiments, the linker comprises an alanine linker. In some embodiments, the linker (not including the protease cleavage site) comprises less than or equal to about 10, 9, 8, 7, 6, 5, 4, 3, 2, or 1 amino acids. In some embodiments, the linker (not including the protease cleavage site) comprises more than or equal to about 10, 9, 8, 7, 6, 5, 4, 3, 2, or 1 amino acids. In some embodiments, the linker (not including the protease cleavage site) comprises 1, 2, 3, 4, 5, 6, 7, 8, 9, or 10 amino acids. In some embodiments, the linker (not including the protease cleavage site) comprises 1-10, 2-10, 3-10, 4-10, 5-10, 6-10, 7-10, 8-10, 9-10, 1-9, 2-9, 3-9, 4-9, 5-9, 6-9, 7-9, 8-9, 1-8, 2-8, 3-8, 4-8, 5-8, 6-8, 7-8, 1-7, 2-7, 3-7, 4-7, 5-7, 6-7, 1-6, 2-6, 3-6, 4-6, 5-6, 1-5, 2-5, 3- 5, 4-5, 1-4, 2-4, 3-4, 1-3, 2-3, or 1-2 amino acids. In some embodiments, the amino acids are contiguous. In some embodiments, the peptide linker comprises proline-rich sequences, polar residues (e.g., serine, glycine, threonine), stretches of glycine and serine residues. Non-limiting examples of peptide linkers can be found here Chen, Xiaoying, Jennica L. Zaro, and Wei-Chiang Shen. “Fusion protein linkers: property, design and functionality.” Advanced drug delivery reviews 65.10 (2013): 1357-1369, which is hereby incorporated by reference in its entirety.

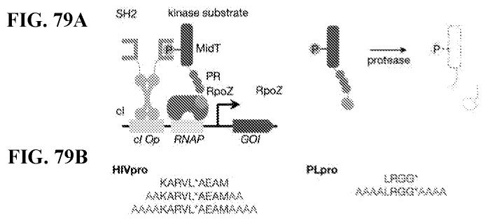

In some embodiments, protease recognition motif comprises an amino acid sequence that is capable of being hydrolyzed by the 3CL protease (3CLpro) of severe acute respiratory syndrome coronavirus 2 (SARS-COV-2). In some embodiments, the amino acid sequence comprises AVLQSGFR (SEQ ID NO: 1), which is a substrate recognition motif for 3CLsubs. In some embodiments, the amino acid sequence further comprises a linker sequence. In some embodiments, the linker sequence comprises at least about 1, 2, 3, or 4 alanine residues on the N- and/or C-terminal sides of the protease cleavage site. In some embodiments, the protease cleavage site comprises a modification relative to SEQ ID NO: 1. In some embodiments, the modification is an insertion, a substitution, or a deletion of one or more amino acids in SEQ ID NO: 1. In some embodiments, the modification is at an amino acid position 1, 2, 3, 4, 5, 6, 7, or 8 of SEQ ID NO: 1. In some embodiments, the protease cleave site is indicated by an “*”, such as for example, in FIG. 79B. In some embodiments, the linker or the protease cleave site or both comprises an insertion. In some embodiments, the insertion comprises 1-10, 2-10, 3-10, 4-10, 5-10, 6-10, 7-10, 8-10, 9-10, 1-9, 2-9, 3-9, 4-9, 5-9, 6-9, 7-9, 8-9, 1-8, 2-8, 3-8, 4-8, 5-8, 6-8, 7-8, 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, or 1-2 amino acids. In some embodiments, the insertion comprises 1, 2, 3, 4, 5, 6, 7, 8, 9, or 10 amino acids. In some embodiments, the insertion at the protease cleavage site enhances recognition by 3CLpro, thereby improving the sensitivity of the system to detect a presence of bioactive molecules modulating the protease in the cell.

In some embodiments, the protease recognition motif comprises an amino acid sequence capable of being hydrolyzed by human immunodeficiency virus 1 protease (HIV-1pro). In some embodiments, the amino acid sequence comprises KARVLAEAM (SEQ ID NO: 2), which is a substrate recognition motif for HIV-1pro. In some embodiments, the amino acid sequence further comprises a linker sequence. In some embodiments, the linker sequence comprises at least about 1, 2, 3, or 4 alanine residues on the N- and/or C-terminal sides of protease cleavage site. In some embodiments, the protease cleavage site comprises a modification relative to SEQ ID NO: 2. In some embodiments, the modification is an insertion, a substitution, or a deletion of one or more amino acids in SEQ ID NO: 2. In some embodiments, the modification is at an amino acid position 1, 2, 3, 4, 5, 6, 7, 8, or 9 of SEQ ID NO: 2. In some embodiments, the protease cleave site is indicated by an “*”, such as for example, in FIG. 79B. In some embodiments, the linker or the protease cleave site or both comprises an insertion. In some embodiments, the insertion comprises 1-10, 2-10, 3-10, 4-10, 5-10, 6-10, 7-10, 8-10, 9-10, 1-9, 2-9, 3-9, 4-9, 5-9, 6-9, 7-9, 8-9, 1-8, 2-8, 3-8, 4-8, 5-8, 6-8, 7-8, 1-7, 2-7, 3-7, 4-7, 5-7, 6-7, 1-6, 2- 6, 3-6, 4-6, 5-6, 1-5, 2-5, 3-5, 4-5, 1-4, 2-4, 3-4, 1-3, 2-3, or 1-2 amino acids. In some embodiments, the insertion comprises 1, 2, 3, 4, 5, 6, 7, 8, 9, or 10 amino acids. In some embodiments, the insertion at the protease cleavage site enhances recognition by HIV-1pro, thereby improving the sensitivity of the system to detect a presence of bioactive molecules modulating the protease in the cell. In some embodiments, the insertion comprises a native recognition site of HIV-1pro. In some embodiments, the insertion comprises a nonnative recognition site of HIV-1 pro.

In some embodiments, the protease recognition motif comprises an amino acid sequence capable of being hydrolyzed by papain-like protease (PLpro). In some embodiments, the amino acid sequence comprises LRGG (SEQ ID NO: 3), which is a substrate recognition motif for PLpro. In some embodiments, the amino acid sequence further comprises a linker sequence. In some embodiments, the linker sequence comprises at least about 1, 2, 3, or 4 alanine residues on the N- and/or C-terminal sides of protease cleavage site. In some embodiments, the protease cleavage site comprises a modification relative to SEQ ID NO: 3. In some embodiments, the modification is an insertion, a substitution, or a deletion of one or more amino acids in SEQ ID NO: 3. In some embodiments, the modification is at an amino acid position 1, 2, 3, or 4 of SEQ ID NO: 3. In some embodiments, the protease cleave site is indicated by an “*”, such as for example, in FIG. 79B. In some embodiments, the linker or the protease cleave site or both comprises an insertion. In some embodiments, the insertion comprises 1-10, 2-10, 3-10, 4-10, 5-10, 6-10, 7-10, 8-10, 9-10, 1-9, 2-9, 3-9, 4-9, 5-9, 6-9, 7-9, 8-9, 1-8, 2-8, 3-8, 4-8, 5-8, 6-8, 7-8, 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, or 1-2 amino acids. In some embodiments, the insertion comprises 1, 2, 3, 4, 5, 6, 7, 8, 9, or 10 amino acids. In some embodiments, the insertion at the protease cleavage site enhances recognition by PLpro, thereby improving the sensitivity of the system to detect a presence of bioactive molecules modulating the protease in the cell.

In some embodiments, the insertion comprises the ubiquitin protein. In some embodiments, the insertion comprises a native recognition site for PLpro. In some embodiments, the insertion comprises a nonnative recognition site for PLpro.

Thus, by adding protease recognition motifs to the phosphorylation sensitive B2H system disclosed herein, the inventors of the instant disclosure modified the system to detect inhibitors of proteases rather than phosphatases. In some embodiments, ribosomal binding sites (RBS) were added to the two-hybrid system to enhance ribosomal binding to the mRNA encoding the protease described elsewhere, which had the strongest influence on dynamic range. In some embodiments, the RBS sequences are provided in SEQ ID NOS: 38-42. In some embodiments, the RBS sequences are greater than or equal to about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOS: 38-42. In some embodiments, the RBS is engineered. In some embodiments, the RBS is located to induce transcription of the RNA polymerase described elsewhere. In some embodiments, the RBS is located in the untranslated region in the 5′ direction of the RNA polymerase described elsewhere. In some embodiments, a luminescence-based screen was used to facilitate a rapid evaluation of whether the RBS that were added improved translation of the protease. In some embodiments, a fluorescence-based assay is used to evaluate whether the RBS improved translation of the gene of interest. In some embodiments; growth-coupled assays were used to evaluate whether the two-hybrid system had successfully been modified to detect inhibitors of proteases rather than phosphatases. Methods for screening both components in combination—and, ideally, within the final two-hybrid system intended for use in high-throughput assays—could accelerate the optimization of new protease-specific two-hybrid systems.

In addition, it was discovered that phosphorylation sensitive B2H systems disclosed herein may not require a protease cleavage site to detect inhibitors of proteases given the promiscuity of proteases and the sensitivity of the B2H systems. Thus, in some embodiments, the linker does not comprise a protease cleavage site or recognition motif.

The two-hybrid (e.g., B2H) system described herein has several important advantages over previous biosensors for protease inhibitors, including but not limited to: (i) the substrate-RpoZ fusion being able to accommodate a large range of linker lengths (e.g., the addition of peptide stretches of 4-75 amino acids) and, thus, facilitating the incorporation of different protease cleavage sites; (ii) the system controls the transcription of user-defined GOIs (e.g., genes for luminescence, antibiotic resistance, or, perhaps, fluorescence) and thus, is compatible with a large variety of high-throughput screens; (iii) the system relies on a system of adjustable components—from the protease cleave site and protease RBS, which helped improve dynamic range in the systems, to the peptide substrate and kinase RBS, which can modulate the extent of protein-protein binding, and these components provide multiple routes to two-hybrid optimization. In general, the modularity of the two-hybrid system facilitates its extension to different targets, signals, and assay types.

The screen of terpenoid pathways highlights important challenges and opportunities for using genetically encoded detection systems. A previously unreported terpenoid inhibitor of 3CLpro, α-bisabolol, which has a reasonable IC50 (˜30-80 μM) for a 15-carbon hydrocarbon, was identified. The production of this terpenoid alone, however, was insufficient to enhance antibiotic resistance, which has two implications: (i) that simple comparisons of the product profiles of hits and non-hits can miss inhibitory products and, thus, highlights the importance of including multiple pathways that generate the same product in starting libraries, and (ii) that the survival advantage conferred by some pathways might peak at intermediate production levels—which could plausibly inhibit the target while avoiding off-target interactions—and, thus, motivates a systematic study of inhibitor-generating pathways under different levels of induction. Curiously, one hit identified in the screen (Q41594) produced small amounts of α-bisabolol in liquid culture, where intracellular titers were lower than the IC50, as described below with respect to Examples 12-14. These titers, which varied with media composition, motivate future efforts to screen and analyze pathways under identical growth conditions. By whittling down large pathway libraries such as those described herein to a small subset that generate inhibitors, they can reduce the throughput required for compound isolation and analysis.

d. Gene of Interest (GOI)

Provided herein, in some embodiments, are genes of interest (GOI), which refer to genes capable of producing a gene expression product that is detectable directly or indirectly. In some embodiments, the GOI encodes a detectable polypeptide, such as a fluorescent polypeptide, or an amplifying enzyme (e.g., T7 RNA polymerase). Non-limiting examples of fluorescent polypeptides comprise, but are not limited to green fluorescent protein, enhanced green fluorescent protein, green fluorescent protein ultra violet, blue fluorescent protein, enhanced blue fluorescent protein yellow fluorescent protein, enhanced yellow fluorescent protein, red fluorescent protein, DsRed fluorescent protein, cyan fluorescent protein, enhanced cyan fluorescent protein, mCherry, m Turquoise, m Venus, mRuby, mWasabi, mTagBFP, mCitrine, mBanana, mOrange, dTomato, and Emerald. In some embodiments, the GOI encodes an enzyme that produces a detectable signal when introduced to a substrate, such as for example, luciferase, β-galactosidase, or bacterial luminescence (lux). In some embodiments, the GOI encodes a gene expression product that confers antibiotic resistance. Non-limiting examples of GOI that confer antibiotic resistance include SpecR, beta-lactamases, bleomycin binding protein Ble-MBL, blasticidin S deaminase, aminoglycoside adenylyltransferase, aminoglycoside phosphotransferase, tetracycline efflux protein, puromycin N-acetyltransferase, chloramphenicol acetyltransferase, neomycin phosphotransferase II, sterol 24-C-methyltransferase, bifunctional enzyme AAC/APH, or mobilized colistin resistance. In some embodiments, the amino acid sequence for SpecR comprises SEQ ID NO: 79. In some embodiments, the amino acid sequence for SpecR is greater than or equal to 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 79. In some the GOI comprises LuxAB. In some embodiments, the amino acid sequence for LuxAB comprises SEQ ID NO: 34. In some embodiments, the amino acid sequence for LuxAB is greater than or equal to 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 34.

In some embodiments, the GOI encodes a transcriptional repressor. In some embodiments, the GOI encodes a catalytically dead Cas protein. In some embodiments, the GOI encodes transcription repressor such as tetracycline repressor, LexA repressor, lacI repressor, Centromere Binding Factor 1 (CBF1), Krüppel-associated box (KRAB). In some embodiments, the repressor encodes SrpR, AmeR, BetI, PsrA, PhiF or HlyII. In some embodiments, the repressor is derived from a bacteria, yeast, tetrapod, insect, plant, or mammal.

### 3. Bioactive Molecules

Provided herein are bioactive molecules produced by a genetically modified organism disclosed herein, which may or may not utilize a combination of complex metabolic pathways that work together to produce the bioactive molecule. In some embodiments, the bioactive molecule is a potential therapeutic agent, which may be useful for treating a disease or a condition disclosed herein.

In some embodiments, the bioactive molecule is a modulator of the target enzyme. In some embodiments, the modulator of the target enzyme is an inhibitor of the target enzyme. In some embodiments the inhibitor of the target enzyme is an allosteric modulator of the target enzyme. In some embodiments, the modulator of the target enzyme is an agonist of the target enzyme. In some embodiments the agonist of the target enzyme is an allosteric modulator of the target enzyme. In some embodiments, the modulator of the target enzyme binds the target enzyme directly or indirectly. Non-limiting examples of methods of analysis of protein-protein binding to determine whether the modulator binds the target enzyme include a co-immunoprecipitation (co-IP), pull-down, crosslinking protein interaction analysis, labeled transfer protein interaction analysis, or Far-western blot analysis, FRET based assay, including, for example FRET-FLIM, a yeast two-hybrid assay, BiFC, or split luciferase assay.

In some cases, the metabolic pathway may be known or unknown; the genetically engineered systems and methods of the present disclosure may be driven (e.g., through evolutionary selection) to find a combination of metabolic pathways to arrive at a desirable bioactive molecule. A bioactive molecule may comprise various classes of biologically produced molecules, where “classes” may refer to any named category that defines a group of molecules having a common characteristic (e.g., proteins, nucleic acids, carbohydrates, small molecule). In some cases, a bioactive molecule may undergo various modifications and/or transformations to its structure. For example, a bioactive protein molecule may be modified with various post-translational modifications and/or transform in conformation (which may be guided by other proteins such as chaperons, heat shock proteins, and any protein that serves a folding function).

A bioactive molecule may comprise one or a combination of molecular components from various biomolecule classes, for example, metabolites (e.g., terpenoids, peptides, or phenylpropanoids), amino acids, carbohydrates, nucleic acids, lipids, any monomeric forms thereof, any polymeric forms thereof, or any derivatives thereof. In some embodiments, a bioactive molecule may comprise one or more modifications. For example, a bioactive protein may comprise post-translation modifications, including, but not limited to: acylation, myristoylation, palmitoylation, isoprenylation, prenylation, farnesylation, geranylgeranylation, glypiation, glycosylphosphatidylinositol anchor formation, lipoylation, flavin functionalization, heme functionalization, phosphorylation, phosphopantetheinylation, retinylidene Schiff base formation, diphthamide formation, ethanolamine phosphoglycerol functionalization, hypusine formation, beta-Lysine addition, acetylation, formylation, alkylation, methylation, amidation, amide bond formation, butyrylation, gamma-carboxylation, glycosylation, polysialylation, malonylation, hydroxylation, iodination, nucleotide addition, phosphate ester formation, phosphoramidate formation, adenylation, uridylylation, propionylation, pyroglutamate formation, gluthathionylation, nitrosylation, sulfenylation, sulfinylation, sulfonylation, succinylation, sulfation, glycation, carbamylation, carbonylation, isopeptide bond formation, biotinylation, carbamylation, oxidation, pegylation, citrullination, deamidation, eliminylation, disulfide bond formation, proteolytic cleavage, isoaspartate formation, racemization, protein splicing, chaperon-assisted folding.

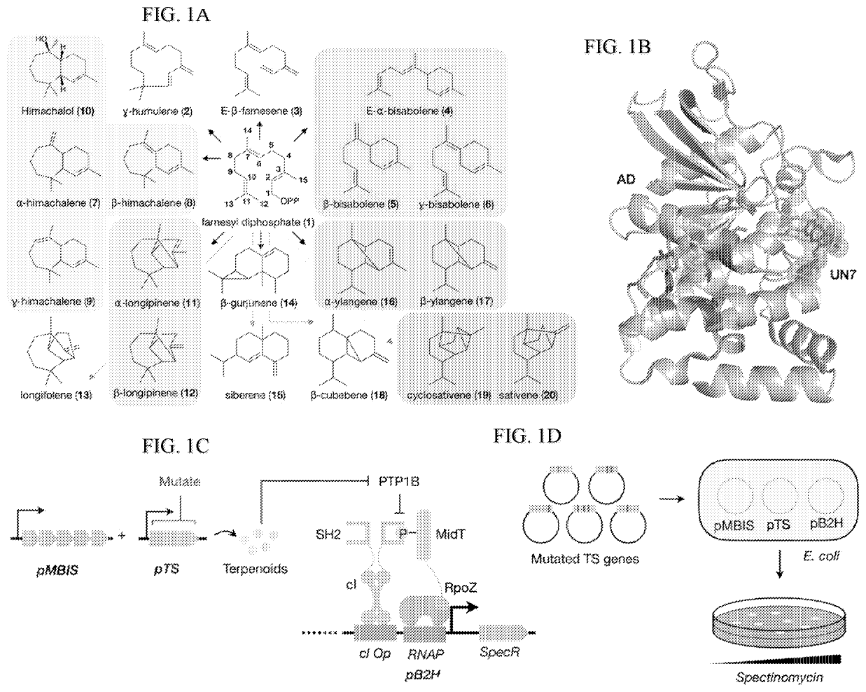

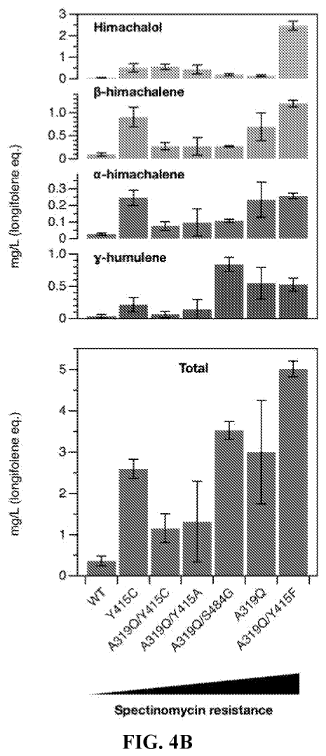

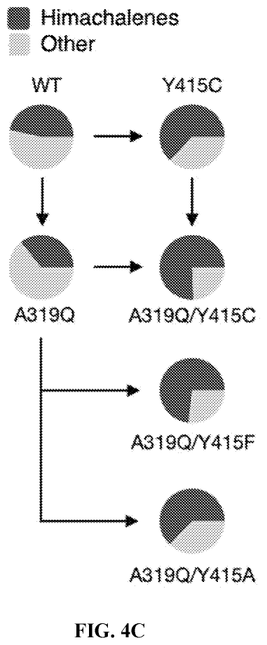

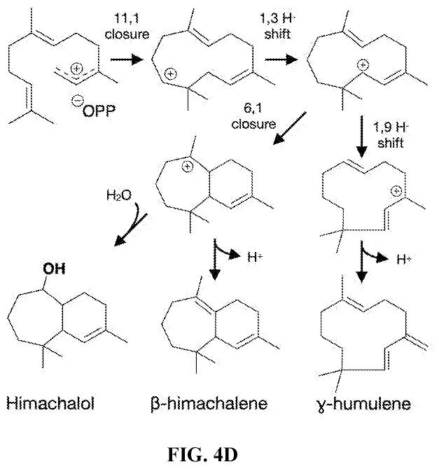

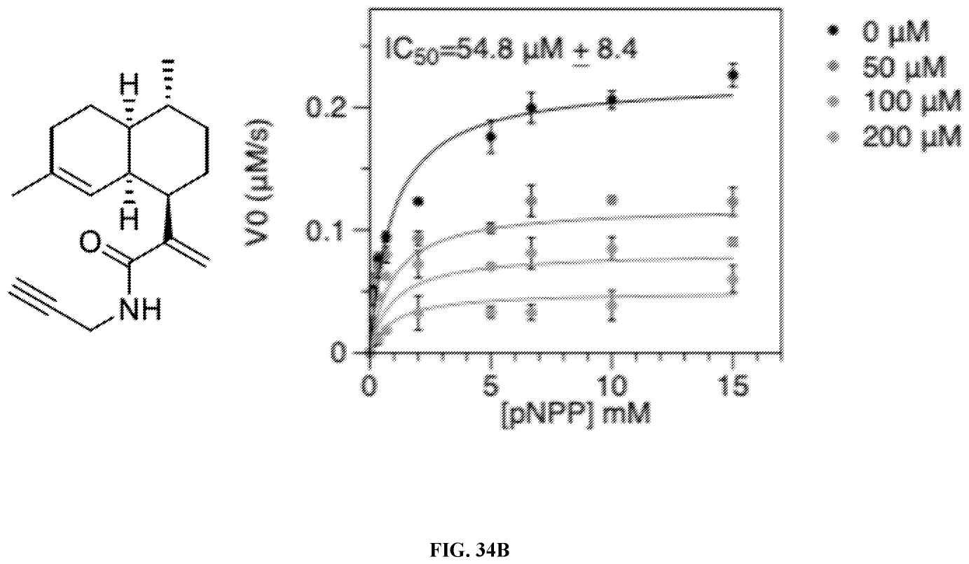

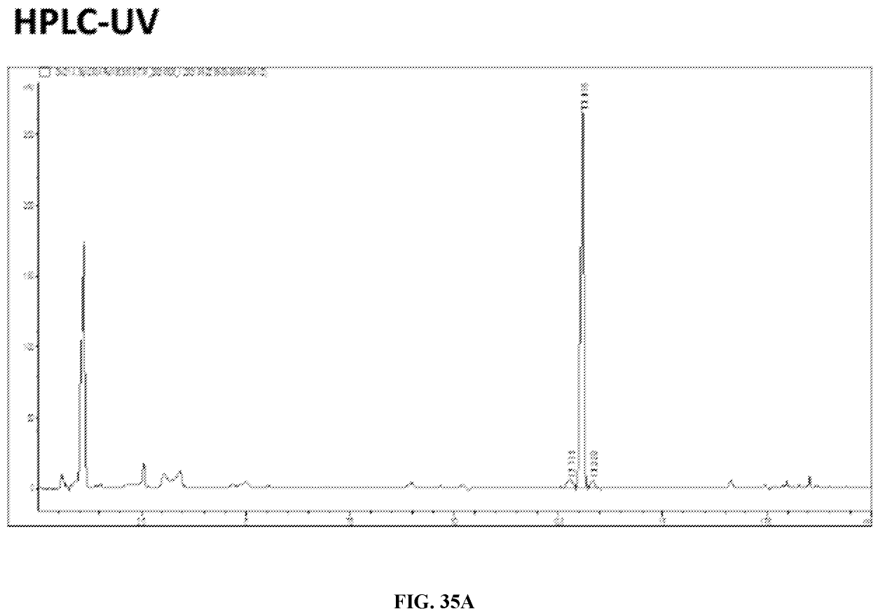

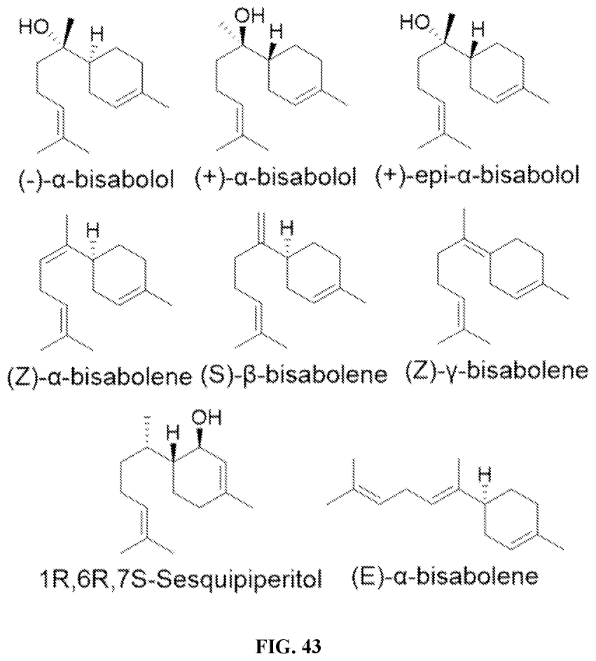

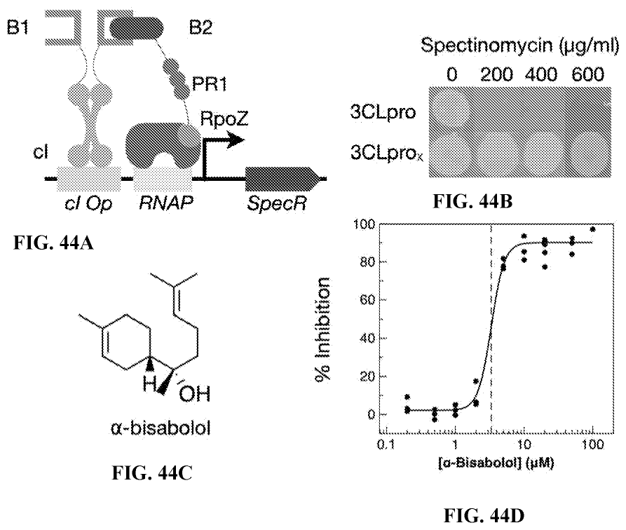

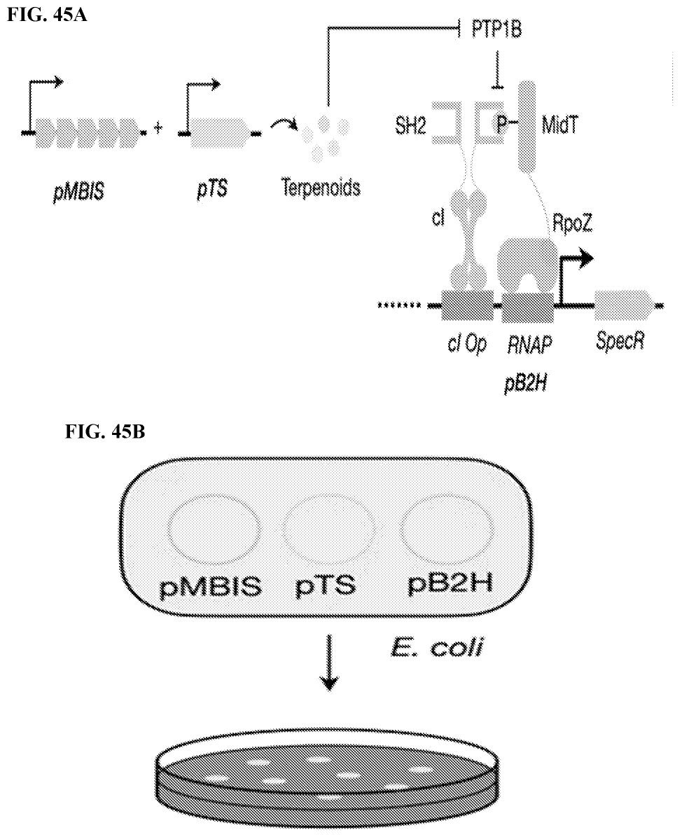

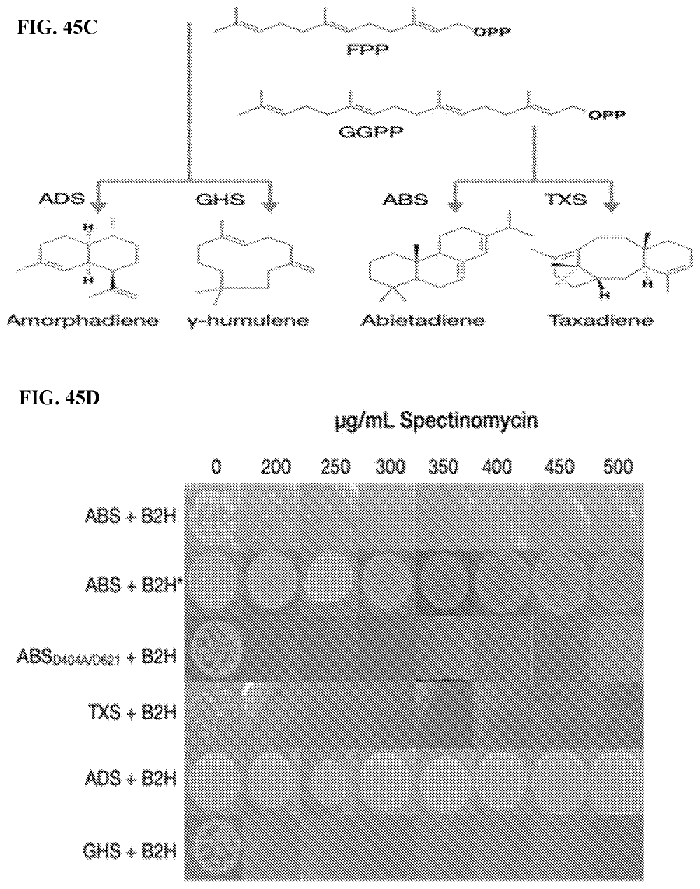

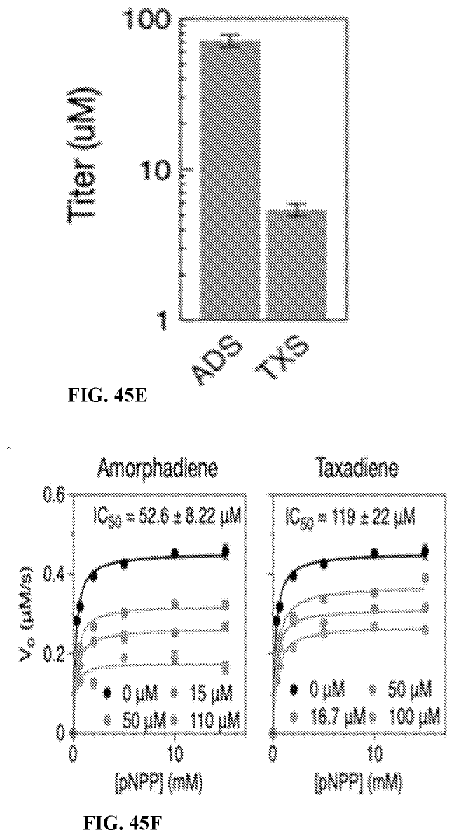

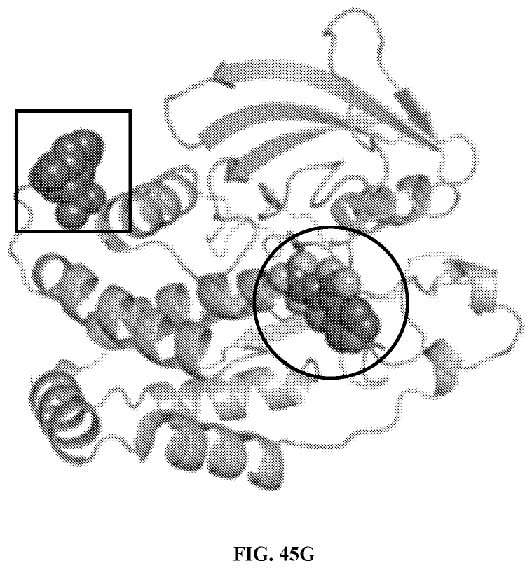

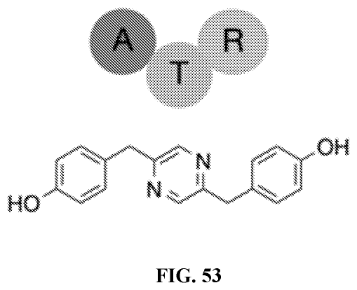

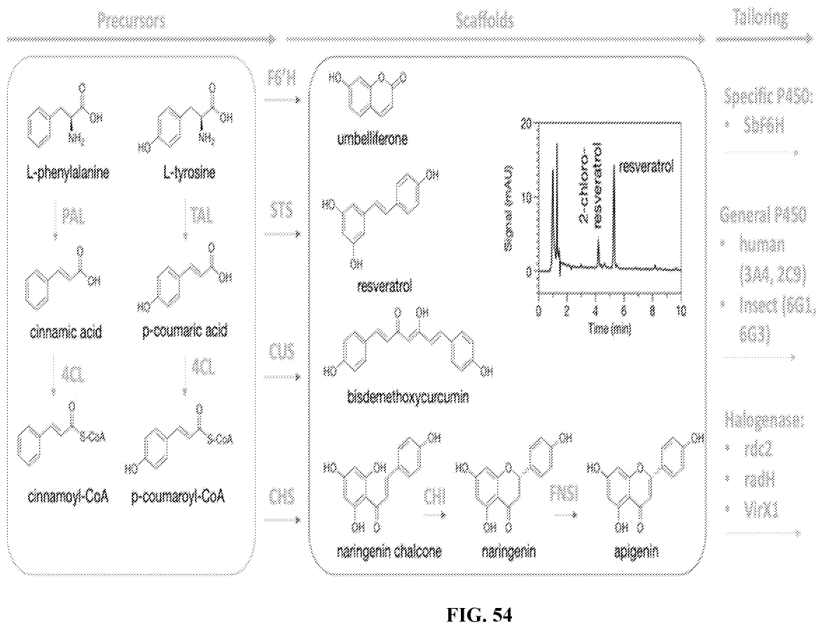

In some embodiments, the bioactive molecule comprises a chemical compound. In some embodiments, the bioactive molecule comprises an intermediate of a metabolic pathway, such for example, farnesyl diphosphate. In some embodiments, the bioactive molecule comprises a sesquiterpene. In some embodiments, the bioactive molecule comprises Himachalol, β-himachalene, γ-humulene, E-β-farnesene, E-α-bisabolene, β-bisabolene, γ-bisabolene, α-himachalene, γ-himachalene, α-longipinene, β-gurjunene, α-ylangene, β-ylangene, longifolene, β-longipinene, siberene, β-cubebene, cyclosativene, or sativene, or any combination thereof, as shown in FIG. 1. In some embodiments, the bioactive molecule comprises Himachalol, β-himachalene, γ-humulene, or any combination thereof, as shown in FIG. 4D. In some embodiments, the bioactive molecule comprises α-bisabolol, or a derivative thereof. In some embodiments, the bioactive molecule comprises amorphadiene, or a derivative thereof (e.g., a propargyl derivative of amorphadiene), as shown in FIG. 34A-34B. In some embodiments, the bioactive molecule comprises abietadiene, Taxadiene, γ-humulene, or amorphadiene. In some embodiments, the bioactive molecule comprises the structure provided in FIG. 43, or a derivative thereof. In some embodiments, the bioactive molecule comprises (−)-α-bisabolol, (+)-α-bisabolol, (+)-epi-α-bisabolol, (Z)-α-bisabolene, (S)-β-bisabolene, (Z)-γ-bisabolene, 1R,6R,7S-Sesquipiperitol, or (E)-α-bisabolene, or a derivative thereof. In some embodiments, the bioactive molecule comprises (−)-α-bisabolol, (+)-α-bisabolol, (+)-epi-α-bisabolol, or α-bisabolol, or a combination thereof. In some embodiments, the bioactive molecule comprises eucalyptol. In some embodiments, the bioactive molecule comprises a pyrazine dipeptide, such as for example, the pyrazine dipeptide in FIG. 53. In some embodiments, the bioactive molecule comprises a precursor, a scaffold, or a combination thereof, shown in FIG. 54. In some embodiments, the bioactive molecule comprises α-bisabolol, β-bisabolene, Eucalyptol, Indole, Amorphadiene, Amorphen-3-en-9-ol, trans-Nerolidol, or Zingiberol, or any combination thereof.

In some embodiments, the bioactive molecule is a flavonoid. In some embodiments the flavonoid is a phenylpropanoid. In some embodiments, the phenylpropanoid comprises L-phenylalanine, L-tyrosine, cinnamic acid, p-coumaric acid, coumarin, umbelliferone, pinosylvin, resveratrol, pinocembrin, naringenin chalcone, naringenin, pinocembrin, chrysin, apigenin, baicalein, scutellarein, or a combination thereof. In some embodiments, the bioactive molecule is a nonribosomal peptide. In some embodiments, the peptide is an aldehyde. In some embodiments, the peptide is a dipeptide. In some embodiments, the dipeptide has a dipeptide pyrazine core. In some embodiments the dipeptide is an aldehyde.

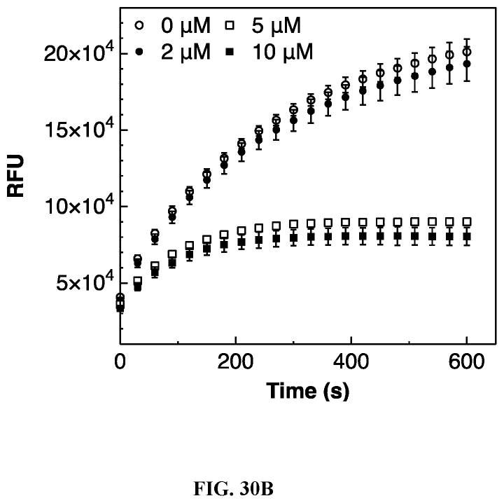

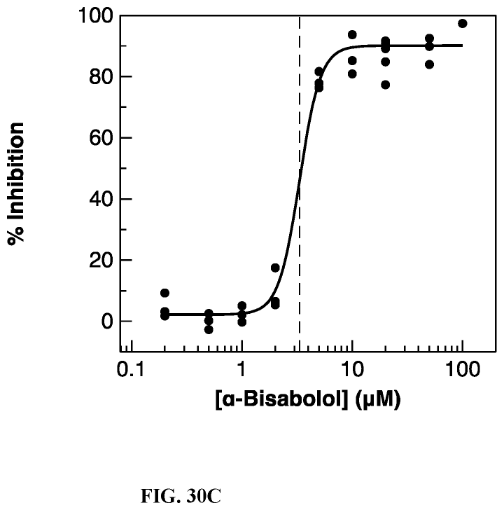

In some embodiments, the bioactive molecule inhibits the target enzyme with a percent (%) inhibition that is greater than or equal to about 90%. In some embodiments, the bioactive molecule inhibits the target enzyme with a percent (%) inhibition that is equal to about 90%, 91%, 92%, 93%, 94%, 95%, 96%, 97%, 98%, 99%, or 100%. In some embodiments, the bioactive molecule inhibits the target enzyme with a percent (%) inhibition that is greater than or equal to about 70%, 75%, 80%, 85%, 86%, 87%, 88%, 89%, 90%, 91%, 92%, 93%, 94%, 95%, 96%, 97%, 98%, or 99%. In some embodiments, the bioactive molecule inhibits the target enzyme with a percent (%) inhibition that is from 70%-100%, 75%-95%, or 80%-90%. In some embodiments, the bioactive molecule inhibits the target enzyme with a percent (%) inhibition that is from 80%-100%. In some embodiments, the bioactive molecule activates the target enzyme with a percent (%) inhibition that is greater than or equal to about 90%. In some embodiments, the bioactive molecule activates the target enzyme with a percent (%) inhibition that is equal to about 90%, 91%, 92%, 93%, 94%, 95%, 96%, 97%, 98%, 99%, or 100%. In some embodiments, the bioactive molecule activates the target enzyme with a percent (%) inhibition that is greater than or equal to about 70%, 75%, 80%, 85%, 86%, 87%, 88%, 89%, 90%, 91%, 92%, 93%, 94%, 95%, 96%, 97%, 98%, or 99%. In some embodiments, the bioactive molecule activates the target enzyme with a percent (%) inhibition that is from 70%-100%, 75%-95%, or 80%-90%. In some embodiments, the bioactive molecule activates the target enzyme with a percent (%) inhibition that is from 80%-100%. In some embodiments, the bioactive molecule is or comprises α-bisabolol, or a derivative thereof. In some embodiments, percent inhibition or percent activation may be measured using a fluorogenic peptide-based detection system, in which the proteolytic activity of the target enzyme liberates a fluorophore (7-Amino-4-trifluoromethylcoumarin, AFC, λₑₓ=400 nm, λₑₓ=505 nm) from a peptide substrate (TSAVLQ* SEQ ID NO: 81), as shown in FIG. 30B for α-bisabolol.

In some embodiments, the bioactive molecule is present in the cell at a concentration that matches or exceeds the half-maximal inhibitor concentration (IC50) when measured using an in vitro kinetic assay carried out in buffer with purified target enzyme and purified bioactive molecule. In some embodiments, the concentration exceeds the IC50 by about 10%, 20%, 30%, 40%, 50%, 60%, 70%, 80%, 90%, 100%, 200%, 250%, or 300%. In some embodiments, the concentration exceeds the IC50 by about 1-fold, 2-fold, 3-fold, 4-fold, 5-fold, 6-fold, 7-fold, 8-fold, 9-fold, or 10-fold. In some embodiments, the bioactive molecule is present in the cell at a concentration that matches or exceeds the half-maximal activation concentration (AC50) when measured using an in vitro kinetic assay carried out in buffer with purified target enzyme and purified bioactive molecule. In some embodiments, the concentration exceeds the AC50 by about 10%, 20%, 30%, 40%, 50%, 60%, 70%, 80%, 90%, 100%, 200%, 250%, or 300%. In some embodiments, the concentration exceeds the AC50 by about 1-fold, 2-fold, 3-fold, 4-fold, 5-fold, 6-fold, 7-fold, 8-fold, 9-fold, or 10-fold.

### 4. Metabolic Pathways

Disclosed herein, in some embodiments, are metabolic pathways that facilitate the production of bioactive molecules in a cell. In some embodiments, the metabolic pathway comprises a pathway for producing the synthase (e.g., terpene synthase). In some embodiments, the metabolic pathway further comprises a metabolic precursor pathway encoding certain enzymes responsible for producing metabolic precursors that serve as substrates for the synthase to produce the bioactive molecules (e.g., terpenoids). In some embodiments, the metabolic pathway is unknown (e.g., randomized mutagenesis of metabolic components). In some embodiments, the metabolic pathway is known. In some embodiments, the metabolic pathway comprises a mevalonate pathway, a methylerthritol 4-phosphate (MEP) pathway, a deoxyxylulose 5-phosphate (DXP) pathway, or a combination thereof. In some embodiments, the metabolic precursor pathway comprises enzymes that convert mevalonate to isopentyl pyrophosphate (IPP) and farnesyl pyrophosphate (FPP). In some metabolic precursor pathway generates geranyl pyrophosphate (GPP), farnesyl pyrophosphate (FPP), or geranylgeranyl pyrophosphate (GGPP), or any combination thereof. In some embodiments, the metabolic pathway and metabolic precursor pathway are exogenous to the cell. In some embodiments, the metabolic pathway and metabolic precursor pathway are derived from Homo sapiens (human), yeast (e.g., Saccharomyces Cerevisiae), a plant, algae, or bacteria.

In some embodiments, the metabolic pathways comprises isoprenoid precursors isopentenyl diphosphate (IPP), dimethylallyl diphosphate (DMAPP), or a combination thereof. In some embodiments, IPP and DMAPP are synthesized from either (i) acetyl-CoA through the mevalonate pathway (MVA) or (ii) pyruvate and glyceraldehyde 3-phosphate through the non-mevalonate pathway (MEP or DXP). Condensation of IPP and DMAPP generates longer isoprenoids, such as geranyl diphosphate (GPP, C₁₀), farnesyl diophosphate (FPP, C15), or geranylgeranyl diphosphate (GGPP, C₂₀), which are substrates for terpene synthases disclosed herein. In some embodiments, the enzymes encoded by the metabolic pathway comprise mevalonate kinase (ERG12) (NCBI Gene ID: 855248), phosphomevalonate kinase (ERG8) (NCBI Gene ID: 855260), or diphosphomevalonate decarboxylase MVD1 (MVD1) (NCBI Gene ID: 855779), or a combination thereof.

In some embodiments, the metabolic precursor pathway comprises precursors to convert isoprenol into farnesyl diphosphate (FPP) or geranylgeranyl diphosphate (GGPP). In some embodiments, the metabolic pathway further comprises GGPP synthase (GGPPS) that synthesis GGPP from FPP and IPP. In some embodiments, GGPP is a terpenoid precursor for certain terpene synthases disclosed herein, such as α-bisabolene synthase (ABS), or taxadiene synthase (TXS). In some embodiments, FFP is a terpenoid precursor for γ-humulene synthase (GHS), amorphadiene synthase (ADS). Non-limiting examples of encoded metabolic pathways and terpenoid biosynthesis precursors can be found in Martin V J, Pitera D J, Withers S T, Newman J D, Keasling J D. Engineering a mevalonate pathway in Escherichia coli for production of terpenoids. Nat Biotechnol. 2003 July; 21 (7): 796-802; and U.S. patent application Ser. Nos. 17/141,321 and 17/859,509, each of which are hereby incorporated by reference in its entirety.

In some embodiments, the metabolic pathway further includes an enzyme that selectively hydroxylates unactivated carbon-hydrogen bonds. In some embodiments, the enzyme comprises a cytochrome P450 enzyme, a cytochrome P450 reductase enzyme, a cytochrome b5 enzyme, an oxidase enzyme, an acyl transferase enzyme, a glycosyltransferase enzyme, a halogenase, or a peroxidase, or a combination thereof. Non-limiting examples of metabolic pathways that include these enzymes that selectively hydroxylate unactivated carbon-hydrogen bonds are provided in Chang M C, Eachus R A, Trieu W, Ro D K, Keasling J D. Engineering Escherichia coli for production of functionalized terpenoids using plant P450s. Nat Chem Biol. 2007 May; 3 (5): 274-7, which is hereby incorporated by reference in its entirety.

### 5. Synthases

Disclosed herein are synthase enzymes that are engineered to produce a bioactive molecule that modulates the activity or expression of a target enzyme disclosed herein. In some embodiments, the system further comprises a nucleic acid encoding a synthase described herein. In some embodiments, the synthase enzyme has been modified relative to a wild-type (or otherwise unmodified) synthase enzyme. In some embodiments, the modified synthases increase diversity of the bioactive molecules produced by the engineered organism in vivo that modulate the activity or expression of the target enzyme. In some embodiments, the synthase is a terpene synthase or a non-ribosomal peptide synthetase.

In some embodiments, the synthase is derived from a prokaryotic organism. In some embodiments, the prokaryotic organism comprises bacteria, archaea, a virus, or cyanobacteria. In some embodiments, the synthase is derived from a eukaryotic organism. In some embodiments, the eukaryotic organism comprises a plant (e.g., Arabidopsis thaliana), a fungus (e.g., Ascomycetes), algae (e.g., Chlorella, Chlamydomonas), human (Homo sapiens), mouse (Mus musculus), chicken (Gallus gallus), rat (Rattus norvegicus), bovine (Bos taurus), or yeast (e.g., Saccharomyces cerevisiae).

In some embodiments, the terpene synthases disclosed herein are modified to produce terpenoids that modulate a target enzyme disclosed herein as compared with an otherwise wild-type terpene synthases. In some embodiments, the terpene synthases converts GPP, FPP, and/or GGPP (generated by the metabolic precursor pathway) to one or more terpenoids. The modified terpene synthases disclosed herein produce novel terpenoids with therapeutic potential to target enzymes disclosed herein (e.g., protein tyrosine phosphatase, protease). In some embodiments, the terpenoids produced by the terpene synthase inhibit or activate the protein tyrosine phosphatase. In some embodiments, the terpenoids produced by the terpene synthases disclosed herein inhibit or activate a protease disclosed herein.

In some embodiments, the terpene synthase comprises γ-humulene synthase (GHS), amorphadiene synthase (ADS), α-bisabolene synthase (ABS), or taxadiene synthase (TXS). In some embodiments, a wild-type sequence for GHS is SEQ ID NO: 7. In some embodiments, a wild-type sequence for ADS is SEQ ID NO: 4. In some embodiments, a wild-type sequence for TXS is SEQ ID NO: 13. In some embodiments, ABS comprises an amino acid sequence provided in SEQ ID NO: 17.

In some cases, the terpene synthase may comprise a mutated form of GHS, ADS, ABS, or TXS, relative to a wild-type sequence. In some embodiments, the modified terpene synthase comprises a mutation in an amino acid sequence. In some embodiments, the mutation is a single amino acid mutation. In some embodiments, the mutation comprises two or more amino acid mutations. In some embodiments, the terpene synthase may comprise 2, 3, 4, 5, 6, 7, 8, 9, or 10 amino acid mutations. In some embodiments, the terpene synthase may comprise 1-10, 2-9, 3-8, 4-7, or 5-6 amino acid mutations. In some embodiments, the mutation comprises a substitution, insertion, of deletion of one or more amino acids. In some cases, the amino acid sequence comprise at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 7. In some embodiments, the mutation comprises A319Q with reference to SEQ ID NO: 7. In some embodiments, the mutation comprises Y415C with reference to SEQ ID NO: 7. In some embodiments, the mutation comprises a combination thereof. In some embodiments, the mutation comprises (a) A319Q and Y415F, (b) A319Q and S484G, or (c) A319Q and S484G, or a combination thereof, all with reference to SEQ ID NO: 7. In some embodiments, the mutation may comprise an amino acid mutation of an amino acid lacking a hydroxyl group.

In some embodiments, the terpene synthase is truncated such that only the catalytically active portion of the synthase is encoded. In some embodiments, the catalytic portion of GHS is SEQ ID NO: 295. In some embodiments, the catalytic portion of GHS comprises an amino acid sequence that is greater than or equal to about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 295. In some embodiments, a catalytic portion of ADS is SEQ ID NO: 293. In some embodiments, the catalytic portion of ADS comprises an amino acid sequence that is greater than or equal to about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 293. In some embodiments, a catalytic portion of TXS is SEQ ID NO: 297. In some embodiments, the catalytic portion of TXS comprises an amino acid sequence that is greater than or equal to about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 297.

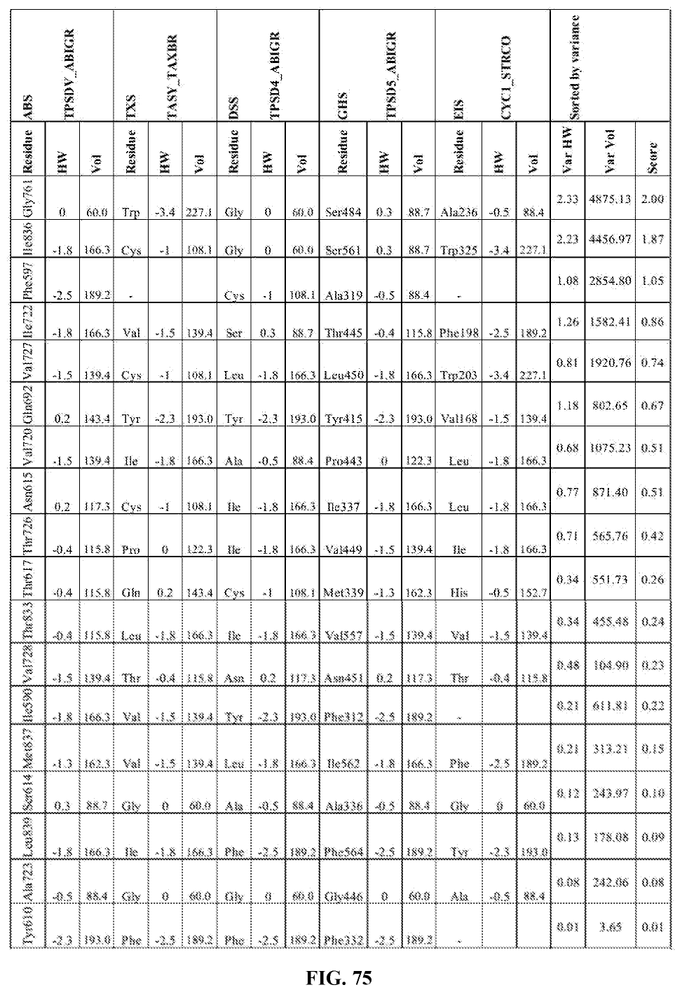

In some embodiments, the terpene synthase comprises one or more mutations provided in FIG. 75. In some embodiments, the terpene synthase comprises a mutation at amino acid positions 484, 561, 319, 445, 450,415, 443, 337, 449, 339, 557, 451, 312, 562, 336, 564, 446, or 332, or any combination thereof with reference to a wild-type sequence. In some embodiments, the GHS comprises a mutation at amino acid positions 484, 561, 319, 445, 450, 415, 443, 337, 449, 339, 557, 451, 312, 562, 336, 564, 446, or 332, or any combination thereof with reference to SEQ ID NO: 7. In some embodiments, the ADS comprises a mutation at amino acid positions 484, 561, 319, 445, 450,415, 443, 337, 449, 339, 557, 451, 312, 562, 336, 564, 446, or 332, or any combination thereof with reference to SEQ ID NO: 4. In some embodiments, the ABS comprises a mutation at amino acid positions 484, 561, 319, 445, 450,415, 443, 337, 449, 339, 557, 451, 312, 562, 336, 564, 446, or 332, or any combination thereof with reference to SEQ ID NO: 17. In some embodiments, the TXS comprises a mutation at amino acid positions 484, 561, 319, 445, 450,415, 443, 337, 449, 339, 557, 451, 312, 562, 336, 564, 446, or 332, or any combination thereof with reference to SEQ ID NO: 13. In some embodiments, the terpene synthase comprises two or more mutations at these amino acid positions. In some embodiments, the terpene synthase comprises 2, 3, 4, 5, 6, 7, 8, 9, or 10 mutations at these amino acid positions.

In some embodiments, the terpene synthase is a catalytically active portion thereof, such as those provided in Table 30. In some embodiments, the catalytically active portion of ADS comprises an amino acid sequence provided in SEQ ID NO: 293. In some embodiments, the catalytically active portion of ADS comprises an amino acid sequence that is greater than or equal to about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 293. In some embodiments, the catalytically active portion of GHS comprises an amino acid sequence provided in SEQ ID NO: 295. In some embodiments, the catalytically active portion of GHS comprises an amino acid sequence that is greater than or equal to about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 295. In some embodiments, the catalytically active portion of TXS comprises an amino acid sequence provided in SEQ ID NO: 297. In some embodiments, the catalytically active portion of TXS comprises an amino acid sequence that is greater than or equal to about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 297.

In some embodiments, the terpene synthase is provided in Table 31. (S)-β-Bisabolene synthase, β-Bisabolene synthase, Taxadiene synthase, Terpene synthase from Cynara cardunculus var, (+)-α-Bisabolol synthase, (+)-epi-α-Bisabolol synthase, γ-Humulene synthase, Sesquiterpene synthase 14b, Artemisia annua (Sweet wormwood) Amorpha-4,11-diene synthase, or a combination thereof. In some embodiments, (S)-β-Bisabolene synthase comprises an amino acid sequence provided in SEQ ID NO: 9. In some embodiments, (S)-β-Bisabolene synthase comprises an amino acid sequence that is greater than or equal to about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 9. In some embodiments, β-Bisabolene synthase comprises an amino acid sequence provided in SEQ ID NO: 11. In some embodiments, β-Bisabolene synthase comprises an amino acid sequence that is greater than or equal to about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 11. In some embodiments, Terpene synthase from Cynara cardunculus var comprises an amino acid sequence provided in SEQ ID NO: 15. In some embodiments, Terpene synthase from Cynara cardunculus var comprises an amino acid sequence that is greater than or equal to about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 15. In some embodiments, (+)-α-Bisabolol synthase comprises an amino acid sequence provided in SEQ ID NO: 17. In some embodiments, (+)-α-Bisabolol synthase comprises an amino acid sequence that is greater than or equal to about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 17. In some embodiments, (+)-epi-α-Bisabolol synthase comprises an amino acid sequence provided in SEQ ID NO: 19. In some embodiments, (+)-epi-α-Bisabolol synthase comprises an amino acid sequence that is greater than or equal to about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 19. In some embodiments, γ-Humulene synthase comprises an amino acid sequence provided in SEQ ID NO: 7. In some embodiments, γ-Humulene synthase comprises an amino acid sequence that is greater than or equal to about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 7. In some embodiments, Sesquiterpene synthase 14b comprises an amino acid sequence provided in SEQ ID NO: 23. In some embodiments, Sesquiterpene synthase 14b comprises an amino acid sequence that is greater than or equal to about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 23. In some embodiments, Artemisia annua (Sweet wormwood) Amorpha-4,11-diene synthase comprises an amino acid sequence provided in SEQ ID NO: 4. In some embodiments, Artemisia annua (Sweet wormwood) Amorpha-4,11-diene synthase comprises an amino acid sequence that is greater than or equal to about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 4.

In some embodiments, the non-ribosomal peptide synthetase comprises a carrier protein domain, an adenylation domain, a condensation domain, a thioesterase domain, or a reductase domain, or a combination thereof. Non-limiting examples of non-ribosomal peptide synthetases and their substrates are discussed in Miller B R, Gulick A M. “Structural Biology of Nonribosomal Peptide Synthetases.” Methods Mol Biol. 1401 (2016) 3-29, which is hereby incorporated by reference. In some embodiments, the non-ribosomal peptide synthetase comprises GupB, Nterp, or a combination thereof. In some embodiments, the non-ribosomal peptide synthetase is a dipeptide synthase. In some embodiments, the non-ribosomal peptide synthetase is a cyclodipeptide synthase. In some embodiments the non-ribosomal peptide synthetase comprises domains from one or more naturally occurring non-ribosomal peptide synthetases. In some embodiments the non-ribosomal peptide synthase has one or more mutations in one or more adenylation (A) domains. In some embodiments, the non-ribosomal peptide synthase includes one or more adenylation (A) domains from a different source organism than other domains in the non-ribosomal peptide synthase.

In some cases, the one or more products of the terpene synthase are isolated. In some embodiments, the one or more products of the terpene synthase are purified. In some embodiments, the terpene synthase or modified terpene synthase, or catalytically active portion thereof is isolated or purified.

Provided herein are methods of amplifying expression of a reporter in vivo that may be linked to inhibition of a target enzyme. In some embodiments, the GOI encodes an enzyme capable of inducing expression of a detectable polypeptide disclosed herein, such as a polymerase. In some embodiments, the GOI encodes T7 RNA polymerase. Other non-limiting examples of RNA polymerases include other viral RNA polymerases, such as T3 polymerase, SP6 polymerase, and Kl l polymerase; Eukaryotic RNA polymerases, such as such as RNA polymerase I, RNA polymerase II, RNA polymerase III, RNA polymerase IV, and RNA polymerase V; or Archaea RNA polymerases. This polymerase encoded by the GOI can then bind to the promoter driving expression of a detectable polypeptide, resulting in some cases, in amplification of the detectable signal by nearly 5-fold, as compared to the GOI encoding the detectable polypeptide itself.

### 6. Nucleic Acid Molecules Encoding the Genetically-Encoded Systems

Provided herein, in some embodiments, are one or more nucleic acid molecules encoding the systems disclosed herein. In some embodiments, the nucleic acid molecules comprise deoxyribonucleic acid (DNA) or ribonucleic acid (RNA). In some embodiments, the one or more nucleic acid molecules encoding the target enzymes comprise a plasmid vector, a viral vector, a cosmid, an artificial chromosome, or a region of the host chromosome. In some embodiments, the plasmid vector is derived from bacteria, archaea, yeast, or plants. In some embodiments, the viral vector is derived from adenovirus, adeno-associated virus, retrovirus, lentivirus, poxvirus, baculovirus, or herpes simplex virus. In some embodiments, the one or more nucleic acid molecules encode a phosphorylated protein binding domain, a kinase substrate, a repressor element, a subunit of RNA polymerase or portions thereof, a kinase, the kinase/phosphatase substrate, the target enzyme (e.g., protease, phosphatase), an operator for the repressor element, binding site for the subunit of RNA polymerase or portions thereof, a chaperone polypeptide, a metabolic pathway, synthase (e.g., terpene synthase), a gene of interest (GOI), or any combination thereof.

In some embodiments, the systems disclosed herein comprise a single nucleic acid molecule encoding the phosphorylated protein binding domain, the repressor element, the subunit of RNA polymerase or portions thereof, the kinase, the kinase/phosphatase substrate, the target enzyme (e.g., protease, phosphatase), the operator for the repressor element, binding site for the subunit of RNA polymerase or portions thereof, the chaperone polypeptide, the metabolic pathway, the synthase (e.g., terpene synthase), the GOI, or any combination thereof. In some embodiments, the systems disclosed herein comprise more than one nucleic acid molecule encoding the phosphorylated protein binding domain, the repressor element, the subunit of RNA polymerase or portions thereof, the kinase, the kinase/phosphatase substrate, the target enzyme (e.g., protease, phosphatase), the operator for the repressor element, binding site for the subunit of RNA polymerase or portions thereof, the chaperone polypeptide, the metabolic pathway, the synthase (e.g., terpene synthase), the GOI, or any combination thereof. In some embodiments, the systems disclosed herein comprise 2, 3, 4, 5, 6, 7, 8, 9, 10 or more nucleic acid molecules. In some embodiments, the two-hybrid system comprises two separate nucleic acid molecules. For example, the two-hybrid system may comprise a first nucleic acid molecule (e.g., plasmid vector) encoding the phosphorylated protein binding domain, a repressor element, a subunit of RNA polymerase or portions thereof, the chaperone polypeptide, and the target enzyme; and a second nucleic acid molecule encoding the gene of interest (GOI), and comprising the binding site for the subunit of RNA polymerase or portions thereof, an operator for the repressor element. In some embodiments, the first nucleic acid molecule comprises a ribosomal binding site (RBS) disclosed herein.

Provided herein, in some embodiments, are systems comprising: (1) a first nucleic acid sequence encoding a phosphorylated protein binding domain; (2) a second nucleic acid sequence encoding a repressor element; (3) a third nucleic acid sequence encoding a subunit of RNA polymerase or portions thereof; (4) a fourth nucleic acid sequence encoding a kinase/phosphatase substrate; (5) a fifth nucleic acid sequence encoding kinase; (6) a sixth nucleic acid encoding the target enzyme; (7) a seventh nucleic acid encoding an operator for the repressor element; (8) an eighth nucleic acid sequence comprising a binding site for the RNA polymerase; and (9) a ninth nucleic acid sequence encoding a polymerizing enzyme. In some embodiments, the kinase substrate is coupled to the subunit of the RNA Polymerase or portions thereof. In some embodiments, kinase substrate comprises MidT. In some embodiments, the subunit of the RNA polymerase or portions thereof comprises Rpoz. In some embodiments, there is a linker between the kinase substrate and the subunit of the RNA Polymerase or portions thereof. In some embodiments, the repressor element is coupled to the phosphorylated protein binding domain. In some embodiments the repressor element is or comprises cI repressor. In some embodiments, the phosphorylated protein binding domain is or comprises SH2. In some embodiments, the repressor element and the phosphorylated protein binding domain are coupled by a linker. In some embodiments, the target enzyme comprises a protease, such as those disclosed herein. In some embodiments, the systems further comprise a (10) tenth nucleic acid sequence encoding a metabolic pathway for producing the bioactive molecule described herein. In some embodiments, the systems further comprise (11) an eleventh nucleic acid sequence encoding a synthase enzyme for producing the bioactive molecule. In some embodiments, the eleventh nucleic acid sequence further encodes and enzyme for synthesizing geranylgeranyl diphosphate (GGPP) from metabolic intermediates (e.g., farnesyl diphosphate (FFP), and isopentenyl diphosphate (IPP), e.g., geranylgeranyl diphosphate synthase (GGPPS)). In some embodiments, the first, second, third, fourth, fifth, sixth, seventh and eighth nucleic acid sequences are on a single nucleic acid molecule. In some embodiments, the first, second, third, fourth, fifth, sixth, seventh and eighth nucleic acid sequences are on a single nucleic acid molecule. In some embodiments, the first, second, third, fourth, fifth, sixth and ninth nucleic acid sequences are comprised in a single nucleic acid molecule. In some embodiments, the seventh and eighth nucleic acid sequences are comprised in a single nucleic acid molecule. In some embodiments, the tenth and elevenths nucleic acid sequence may be comprised in a single nucleic acid molecule or more than one.

In some embodiments, the one or more nucleic acid molecules encoding the above genetically-encoded system components comprises a promoter sequence configured to drive expression of a gene expression product. In some embodiments, the gene expression produce comprises the phosphorylated protein binding domain, the repressor element, the subunit of RNA polymerase or portions thereof, the kinase, the kinase/phosphatase substrate, the target enzyme (e.g., protease, phosphatase), the operator for the repressor element, binding site for the subunit of RNA polymerase, the chaperone polypeptide, the metabolic pathway, the synthase (e.g., terpene synthase), the GOI, or any combination thereof. In some embodiments, the one or more nucleic acid molecules comprises an operator or an inducer of transcription of the gene expression product. In some embodiments, the one or more nucleic acid molecules comprises an enhancer, a response element, or a silencer. In some embodiments, one or more nucleic acid molecules comprises, in a 5′ to a 3′ direction, a promoter and a nucleic acid sequence encoding the gene expression product (e.g., a component of the system). In some embodiments, the one or more nucleic acid molecules comprises, in a 5′ to a 3′ direction, a promoter, an operator, and a nucleic acid sequence encoding the gene expression product (e.g., a component of the system). In some embodiments, the one or more nucleic acid molecules is comprised in an operon. In some embodiments, the promoter comprises a TATA Box for forming the transcription initiation complex in a eukaryotic cell. In some embodiments, the promoter comprises a Pribnow box for forming the transcription initiation complex in a bacterial cell.

In some embodiments, the promoter comprises a pBAD promoter, Prol promoter, placZopt promoter, ProD promoter, or any combination thereof. In some embodiments, the promoter comprises a nucleic acid sequence provided in any one of SEQ ID NOS: 82-85. In some embodiments, the promoter comprises a nucleic acid sequence that is greater than or equal to 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOS: 82-85.

Provided herein, in some embodiments, are one or more nucleic acid molecules encoding a repressor element. In some embodiments, the operator for the repressor element comprises a cI repressor. In some embodiments, the cI repressor can be identified with Primary Accession No. P03034 (UniProt) (SEQ ID NO: 86).

Provided herein, in some embodiments, are one or more nucleic acid molecules encoding chaperone polypeptide. In some embodiments, the chaperone polypeptide comprises CDC37. In some embodiments, the one or more nucleic acid molecules encoding CDC37 is provided in SEQ ID NO: 75. In some embodiments, the one or more nucleic acid molecules encoding CDC37 is greater than or equal to about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 75.

Provided herein, in some embodiments, are one or more nucleic acid molecules encoding a subunit of RNA polymerase or portions thereof. In some embodiments, the binding site for the RNA polymerase is a binding site for a subunit of the RNA polymerase or portions thereof (e.g., RpoZ) (SEQ ID NO: 88).

Disclosed herein are one or more nucleic acid molecules encoding a phosphorylated protein binding domain disclosed herein. In some embodiments, the phosphorylated protein binding domain comprises or is a phosphorylated tyrosine binding domain. In some embodiments, the phosphorylated tyrosine binding domain comprises Src homology 2 (SH2). In some embodiments, the one or more molecules comprises a nucleic acid sequence encoding SH2, such as for example SEQ ID NO: 90. In some embodiments, the one or more nucleic acid molecules encoding the SH2 comprises a nucleic acid sequence that is greater than or equal to about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 90.

In some embodiments, the one or more molecules comprises a nucleic acid sequence encoding HA4, such as for example SEQ ID NO: 94. In some embodiments, the one or more nucleic acid molecules encoding the HA4 comprises a nucleic acid sequence that is greater than or equal to about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 94. In some embodiments, the one or more molecules comprises a nucleic acid sequence encoding SH2ABL, such as for example SEQ ID NO:92. In some embodiments, the one or more nucleic acid molecules encoding the SH2ABL comprises a nucleic acid sequence that is greater than or equal to about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO:92.

Disclosed herein are one or more nucleic acid molecules encoding a kinase/phosphatase substrate. In some embodiments, the kinase/phosphatase substrate comprises hamster polyomavirus middle T antigen (MidT). In some embodiments, the one or more molecules comprises a nucleic acid sequence encoding MidT, such as for example SEQ ID NO: 96 or SEQ ID NO: 98. In some embodiments, the one or more nucleic acid molecules encoding the MidT comprises a nucleic acid sequence that is greater than or equal to about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 96 or SEQ ID NO: 98.

Provided herein, in some embodiments, are one or more nucleic acid molecules encoding kinase. In some embodiments, the kinase comprises or is Src Kinase. In some embodiments, the one or more molecules comprises a nucleic acid sequence encoding Src Kinase, such as for example SEQ ID NO:73. In some embodiments, the one or more nucleic acid molecules encoding the Src Kinase comprises a nucleic acid sequence that is greater than or equal to about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO:73. In some embodiments, the one or more nucleic acid molecules encodes a truncated Src Kinase. In some embodiments, the Src Kinase comprises an amino acid sequence that is greater than or equal to about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 246. In some embodiments, the one or more nucleic acid molecules encodes a Lck kinase. In some embodiments, the Lck kinase comprises an amino acid sequence that is greater than or equal to about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 247. In some embodiments, the one or more nucleic acid molecules encodes a Fyn kinase. In some embodiments, the Fyn Kinase comprises an amino acid sequence that is greater than or equal to about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 248. In some embodiments, the one or more nucleic acid molecules encodes a Yes kinase. In some embodiments, the Yes kinase comprises an amino acid sequence that is greater than or equal to about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 249. In some embodiments, the one or more nucleic acid molecules encodes an Epha2 kinase. In some embodiments, the Epha2 kinase comprises an amino acid sequence that is greater than or equal to about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 250. In some embodiments, the one or more nucleic acid molecules encodes a BTK. In some embodiments, the BTK comprises an amino acid sequence that is greater than or equal to about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 251.

Provided herein, in some embodiments, are one or more nucleic acid molecules encoding a target enzyme. In some embodiments, the one or more nucleic acid molecules encoding the target enzymes disclosed herein further comprise a ribosomal binding site (RBS), which enhances translation of the mRNA encoding the target enzyme. In some embodiments, the RBS comprises or is an internal ribosome entry site (IRES). In some embodiments, the RBS comprises 5′-AGGAGG-3′. In some embodiments, the RBS comprises 5′-GGTG-3′. In some embodiments, RBS is modified to further enhance ribosomal binding. In some embodiments, the RBS is engineered via a degenerate primer. In some embodiments, the RBS variants are screened as libraries. In some embodiments, the RBS variants are screened in conjunction with variants in other GOIs or operators (e.g., T7 RNAP, GFPuv). In some embodiments, the RBS is exogenous to the cell. In some embodiments, the RBS is endogenous to the cell. In some embodiments, the RBS is encoded by a nucleic acid sequence comprising any one of SEQ ID NOS: 100-108 or SEQ ID NOS: 39-42. In some embodiments, the RBS is or comprises a nucleic acid sequence that is more than or equal to about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOS: 100-108 or SEQ ID NOS: 39-42.

In some embodiments, the one or more nucleic acid molecules encoding the target enzyme comprises a deoxyribonucleic acid (DNA) sequence encoding the target enzyme. In some embodiments, the DNA sequence encoding PTP1B is provided in SEQ ID NO: 5. In some embodiments, the DNA sequence encoding PTP1B is greater than or equal to about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical SEQ ID NO: 5. In some embodiments, the one or more nucleic acid molecules encodes PTP1B₃₂₁. PTP1B₄₀₅. TCPTP₃₁₇, TCPTP₃₈₇, PEST (E57D)₃₀₆, STEP₂₈₂₋₅₆₃, or SHP₂₂₃₇₋₅₂₉. In some embodiments, the one or more nucleic acid molecules comprises a nucleic acid sequence provided in Table 28. In some embodiments, the one or more nucleic acid molecules comprises a nucleic acid sequence that is greater than or equal to about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of the sequences provided in Table 28. In some embodiments, the one or more nucleic acid molecules encodes a protein kinase. In some embodiments, the one or more nucleic acid molecules comprises a nucleic acid sequence provided in Table 28. In some embodiments, the one or more nucleic acid molecules comprises a nucleic acid sequence that is greater than or equal to about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of the sequences provided in Table 28.

In some embodiments, HIV protease (HIV-1Pr) is encoded by a DNA sequence provided in SEQ ID NO. 62. In some embodiments, HIV-1Pr is encoded by a DNA sequence that is greater than or equal to 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 62. In some embodiments, 3CLpro is encoded by a DNA sequence provided in SEQ ID NO. 68. In some embodiments, 3CLpro is encoded by a DNA sequence that is greater than or equal to 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 68. In some embodiments, NS2B/NS3 protease is encoded by a DNA sequence provided in SEQ ID NO.77. In some embodiments, NS2B/NS3 protease is encoded by a DNA sequence that is greater than or equal to 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 77. In some embodiments PLpro is encoded by a DNA sequence comprising SEQ ID NO: 66. In some embodiments, PLpro comprises is encoded by a DNA sequence that is more than or equal to about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 66. In some embodiments USP7 is encoded by a DNA sequence comprising SEQ ID NO: 64. In some embodiments, USP7 comprises is encoded by a DNA sequence that is more than or equal to about 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 64.

Provided herein, in some embodiments, are one or more nucleic acid molecules encoding a protease cleavage site. In some embodiments, the protease cleavage site is for recognition by 3CLpro. In some embodiments, the one or more nucleic acid molecules encoding the 3CLpro protease cleavage site is provided in SEQ ID NO: 109. In some embodiments, the protease cleavage site is for recognition by HIVpro. In some embodiments, the one or more nucleic acid molecules encoding the HIVpro protease cleavage site is provided in SEQ ID NO:110. In some embodiments, the protease cleavage site is for recognition by PLpro. In some embodiments, the one or more nucleic acid molecules encoding the PLpro protease cleavage site is provided in SEQ ID NO:111. In some embodiments, the protease cleavage site is for recognition by USP7. In some embodiments, the one or more nucleic acid molecules encoding the USP7 protease cleavage site is provided in SEQ ID NO:24.

Provided herein, in some embodiments, are one or more nucleic acid molecules encoding a gene of interest (GOI). In some embodiments, the GOI is or comprises LuxAB. In some embodiments, the one or more nucleic acid molecules encoding LuxAB comprises SEQ ID NO: 112. In some embodiments, the one or more nucleic acid molecules encoding LuxAB is greater than or equal to 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 112. In some embodiments, the GOI is or comprises SpecR. In some embodiments, the one or more nucleic acid molecules encoding SpecR comprises SEQ ID NO:79. In some embodiments, the one or more nucleic acid molecules encoding SpecR is greater than or equal to 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 79.

Provided herein, in some embodiments, are one or more nucleic acid molecules encoding an operator for the repressor element. In some embodiments, the one or more nucleic acid molecules encoding the operator comprises SEQ ID NOS: 113-117. In some embodiments, the one or more nucleic acid molecules encoding the operator is greater than or equal to 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NOS: 113-117.

Provided herein, in some embodiments, are one or more nucleic acid molecules encoding metabolic pathway. In some embodiments, one or more nucleic acid molecules encoding the metabolic pathway encodes an enzyme that catalyzes the condensation of isopentenyl diphosphate (IPP), or dimethylallyl diphosphate (DMAPP), such as a geranylgeranyl diphosphate synthase (GGPPS). In some embodiments, one or more nucleic acid molecules encoding the metabolic pathway further encodes an enzyme that selectively hydroxylates unactivated carbon-hydrogen bonds disclosed herein. In some embodiments, the enzyme comprises a cytochrome P450 enzyme, a cytochrome P450 reductase enzyme, a cytochrome b5 enzyme, an oxidase enzyme, an acyl transferase enzyme, a glycosyltransferase enzyme, a halogenase, and/or a peroxidase. In some embodiments, one or more nucleic acid molecules encoding the metabolic pathway further encodes mevalonate kinase (ERG12) (NCBI Gene ID: 855248), phosphomevalonate kinase (ERG8) (NCBI Gene ID: 855260), or diphosphomevalonate decarboxylase MVD1 (MVD1) (NCBI Gene ID: 855779), or a combination thereof. In some embodiments, the metabolic pathway is encoded by one nucleic acid molecule. In some embodiments, the metabolic pathway is encoded by two separate nucleic acid molecules. In some embodiments, the metabolic pathway is encoded by three separate nucleic acid molecules. In some embodiments, the metabolic pathway is encoded by four separate nucleic acid molecules. In some embodiments, the system comprises a first nucleic acid molecule encoding mevalonate kinase (ERG12), phosphomevalonate kinase (ERG8, or diphosphomevalonate decarboxylase MVD1 (MVD1), or a combination thereof; and a second nucleic acid molecule encoding a synthase disclosed herein. In some embodiments, the second nucleic acid molecule further encodes geranylgeranyl diphosphate synthase (GGPPS). In some embodiments, the first nucleic acid molecule and the second nucleic acid molecules are plasmid vectors in operable combination with one another. Alternatively, the first and second nucleic acid molecules may be on the same plasmid.

Provided herein are one or more nucleic acid molecules encoding the terpene synthases described herein. In some embodiments, the nucleic acid molecules encoding the terpene synthase comprise a plasmid vector, a viral vector, a cosmid, an artificial chromosome, or a region of the host chromosome. In some embodiments, the nucleic acid molecules encoding the terpene synthetases further encodes the metabolic pathway or metabolic precursor pathway disclosed herein. For example, the nucleic acid molecule encoding the terpene synthase may also encode an enzyme that catalyzes the condensation of isopentenyl diphosphate (IPP), or dimethylallyl diphosphate (DMAPP), such as a geranylgeranyl diphosphate synthase (GGPPS). In some embodiments, the nucleic acid encoding the terpene synthase described herein further encodes an enzyme that selectively hydroxylates unactivated carbon-hydrogen bonds disclosed herein. In some embodiments, the enzyme comprises a cytochrome P450 enzyme, a cytochrome P450 reductase enzyme, a cytochrome b5 enzyme, an oxidase enzyme, an acyl transferase enzyme, a glycosyltransferase enzyme, a halogenase, and/or a peroxidase. In some embodiments, the nucleic acid encoding the terpene synthase described herein further encodes mevalonate kinase (ERG12) (NCBI Gene ID: 855248), phosphomevalonate kinase (ERG8) (NCBI Gene ID: 855260), or diphosphomevalonate decarboxylase MVD1 (MVD1) (NCBI Gene ID: 855779), or a combination thereof. In some embodiments, the one or more nucleic acid molecules encoding the terpene synthases are provided in Table 30. In some embodiments, the one or more nucleic acid molecules comprises a nucleic acid sequence that is greater than or equal to about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of the sequences provided in Table 30.

In some embodiments, the system further encodes or comprises various transcription factors, transcription activators, or transcription repressors. In some embodiments, the cell comprises the various transcription factors. In some embodiments, the system further comprises one or more inducers of transcription, such as for example, a substance that binds to a repressor and prevents the repressor from inhibiting transcription. Also provided herein, in some aspects, are molecular barcodes capable of being added to the one or more nucleic acid molecules disclosed herein that enable identification a component of the system disclosed herein using multiplexed sequence analysis. In some embodiments, the nucleic acid molecules disclosed herein comprise a molecular barcode sequence unique to a target enzyme, a synthase, a metabolic pathway, or a combination thereof. In some embodiments, the nucleic acid molecule encoding the target enzyme also comprises a unique barcode sequence that enables identification of the target enzyme. In some embodiments, the nucleic acid molecule encoding the synthase also comprises a unique barcode sequence that enables identification of the synthase. In some embodiments, the barcode is sufficient to identify a target tyrosine phosphatase. In some embodiments, the target enzyme comprises or is a proteolytic enzyme disclosed herein. In some embodiments, the target enzyme comprises or is a protein phosphatase disclosed herein (e.g., tyrosine phosphatase).

In some embodiments, the molecular barcode comprises or is a unique molecular identifier (UMI) comprising a nucleic acid sequence coupled to a 5′ or a 3′ end (or both 5′ and 3′ end) of a nucleic acid sequence encoding a phosphorylated protein binding domain, a repressor element, a subunit of RNA polymerase or portions thereof, a kinase substrate, kinase, the target enzyme, an operator for the repressor element, a synthase (e.g., terpene synthase), or a metabolic pathway, or any combination thereof.

In some embodiments, the molecular barcode has a length comprising from about 5 nucleotides to 25 nucleotides, 6 nucleotides to 24 nucleotides, 7 nucleotides to 23 nucleotides, 8 nucleotides to 22 nucleotides, 9 nucleotides to 21 nucleotides, 10 nucleotides to 20 nucleotides, 11 nucleotides to 19 nucleotides, 12 nucleotides to 18 nucleotides, 13 nucleotides to 17 nucleotides, or 14 nucleotides to 16 nucleotides. In some embodiments, the length of a molecular barcode comprises less than or equal to 25 nucleotides. In some embodiments, the length of a molecular barcode comprises at least or equal to about 1, 2, 3, 4, 5, or 6 nucleotides. In some embodiments, the molecular barcode comprises at least or equal to about 6 nucleotides. In some embodiments, the nucleotides are contiguous.

In some embodiments, the nucleic acid molecules disclosed herein may comprise an adaptor. In some embodiments, the adaptor comprises one or more primer sites, such as a site for sequencing primer or an amplification primer. In some embodiments, the primer is a universal primer. In some embodiments, the adaptor comprises an index site comprising a nucleic acid sequence that may be capable of identifying the sample. In some embodiments, the index site comprise a nucleic acid sequence that has a length comprising from about 5 nucleotides to 25 nucleotides, 6 nucleotides to 24 nucleotides, 7 nucleotides to 23 nucleotides, 8 nucleotides to 22 nucleotides, 9 nucleotides to 21 nucleotides, 10 nucleotides to 20 nucleotides, 11 nucleotides to 19 nucleotides, 12 nucleotides to 18 nucleotides, 13 nucleotides to 17 nucleotides, or 14 nucleotides to 16 nucleotides. In some embodiments, the length of the index site comprises less than or equal to 25 nucleotides. In some embodiments, the length of the index site comprises at least or equal to about 1, 2, 3, 4, 5, or 6 nucleotides. In some embodiments, the index site comprises at least or equal to about 6 nucleotides. In some embodiments, the nucleotides are contiguous. In some embodiments, the adaptor comprises more than one index site. In some embodiments, the adaptor comprises 2, 3, 4, 5, 6, 7, 8, 9, or 10 index sites. In some embodiments, the adaptor comprises one or more of the UMI disclosed herein. A non-limiting example of an adaptor comprises xGen™ Dual Index UMI.

In some embodiments, the adaptors disclosed herein are designed for a specific next generation sequencing platform, such sequences that allow template molecules (for a sequencing reaction) to be immobilized to a solid surface. In some embodiments, the adaptor comprises P5 and P7 sequences suitable for sequencing using Illumina® sequencing-by-synthesis. Non-limiting sequencing platforms comprises bisulfite-free sequencing, bisulfite sequencing, TET-assisted bisulfite (TAB) sequencing, APOBEC-Coupled Epigenetic (ACE) sequencing, high-throughput sequencing, Maxam-Gilbert sequencing, massively parallel signature sequencing, Polony sequencing, 454 pyrosequencing, Sanger sequencing, sequencing-by-synthesis, SOLID sequencing, Ion Torrent™ semiconductor sequencing, DNA nanoball sequencing, Heliscope single molecule sequencing, single molecule real time (SMRT) sequencing, nanopore DNA sequencing, shot gun sequencing, RNA sequencing, Enzyme-assisted Identification of Genome Modification Assay (EnIGMA) sequencing, nanopore sequencing, sequencing-by-binding, or any combination thereof.

In some embodiments, a molecular barcode is not used to demultiplex sequencing data from a multiplex sequencing reaction. In such an embodiment, a nucleic acid sequence encoding a system component may be used to identify the sample, the terpene synthase, the metabolic pathway, or the target enzyme. For example the nucleic acid sequence encoding the terpene synthase may be used to identify the terpene synthase, and so on. In some embodiments, such implementation of the method is particularly suited to long-read sequencing, using platforms such as (but not limited to) SMRTR sequencing, or nanopore DNA sequencing (e.g., Oxford Nanopore) sequencing.

Provided herein are genetically-encoded system comprising gene of interest (GOI) that encodes a polymerizing enzyme, that when expressed, drives expression of a detectable polypeptide in the presence of an inhibitor of the target enzyme. In some embodiments, the genetically-encoded system disclosed herein comprises: (i) a first nucleic acid sequence encoding a phosphorylated protein binding domain (e.g., phosphorylated tyrosine binding domain); (ii) a second nucleic acid sequence encoding a repressor element; (iii) a third nucleic acid sequence encoding a subunit of RNA polymerase or portions thereof; (iv) a fourth nucleic acid sequence encoding a phosphatase substrate (e.g., tyrosine phosphatase substrate); (v) a fifth nucleic acid sequence encoding kinase (e.g., tyrosine kinase); (vi) a sixth nucleic acid encoding the target enzyme; (vii) a seventh nucleic acid encoding an operator for the repressor element; (viii) an eighth nucleic acid sequence comprising a binding site for the RNA polymerase; (ix) a ninth nucleic acid sequence encoding a polymerizing enzyme, that when expressed, drives expression of a detectable polypeptide in the presence of an inhibitor of the target enzyme. In some embodiments, the signal from the detectable polypeptide is amplified by at least 2-fold, 3-fold, 4-fold, 5-fold, 10-fold, or 100-fold, as compared with expression of the detectable polypeptide by the GOI itself. In some embodiments, the signal may be amplified by about 2-fold to 100-fold, 3-fold to 90-fold, 4-fold to 80-fold, 5-fold to 70-fold, 6-fold to 60-fold, 7-fold to 50-fold, 8-fold to 40-fold, 9-fold to 30-fold, 10-fold to 20-fold.

In some embodiments, the one or more nucleic acid molecules disclosed herein comprises molecular witch that enable precise control over the on/off state of the genetically-encoded system (e.g., the two-hybrid system). In some embodiments, the molecular switch is an optical switch. In some embodiments, the two-hybrid system comprises one or more nucleic acid molecules encoding (i) a variant of a light-oxygen-voltage 2 (LOV2) domain that contains a bacterial SsrA peptide and (ii) a modified SspB peptide in place of the substrate and phosphorylation binding domain (SH2 domains). Exposure of LOV2 to light causes a conformational change that exposes the SsrA peptide and enables an SsrA-SspB interaction that promotes transcription of a gene of interest (GOI). In some embodiments, the GOI is or comprises a gene for LuxAB. This type of photo-switchable system is valuable to control the dynamics of the two-hybrid system to improve the production and/or detection of inhibitors. In some embodiments, the GOI comprises a gene for a fluorescent protein. In some embodiments, the fluorescent protein comprises GFP. In some embodiments, the SsraA-SspB interaction is replaced by a different set of protein binding partners modulated by light. In some embodiments these binding partners are BphP1 and PpsR2.

### B. Computer Systems

The methods and systems may utilize or comprise one or more processors or computers. The processor may be a hardware processor such as a central processing unit (CPU), a graphic processing unit (GPU), a general-purpose processing unit, or a computing platform. The processor may be comprised of any of a variety of suitable integrated circuits, microprocessors, logic devices, field-programmable gate arrays (FPGAs) and the like. In some instances, the processor may be a single core or multi core processor, or a plurality of processors may be configured for parallel processing. Although the disclosure is described with reference to a processor, other types of integrated circuits and logic devices are also applicable. The processor may have any suitable data operation capability. For example, the processor may perform 512 bit, 256 bit, 128 bit, 64 bit, 32 bit, or 16 bit data operations. In some embodiments, such processors and computer systems are programmed to perform analysis of sequencing data from the multiplex sequencing analysis described herein. In some embodiments, the processors and computer systems are programed to demultiplex sequencing data, by assigning the one or molecular barcodes to the tepee synthase, the two-hybrid system, or both.

The computer system includes a central processing unit (CPU, also “processor” and “computer processor” herein), which can be a single core or multi core processor, or a plurality of processors for parallel processing. The computer system also includes memory or memory location (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. The memory, storage unit, interface and peripheral devices are in communication with the CPU through a communication bus (solid lines), such as a motherboard. The storage unit can be a data storage unit (or data repository) for storing data. The computer system can be operatively coupled to a computer network (“network”) with the aid of the communication interface. The network can be the Internet, an internet and/or extranet, or an intranet and/or extranet that is in communication with the Internet. The network in some cases is a telecommunication and/or data network. The network can include one or more computer servers, which can enable distributed computing, such as cloud computing. The network 6, in some cases with the aid of the computer system, can implement a peer-to-peer network, which may enable devices coupled to the computer system to behave as a client or a server.

The CPU can execute a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. Examples of operations performed by the CPU can include fetch, decode, execute, and writeback.

In some embodiments, the program or software is for primary sequence data analysis. In some embodiments, the program or software is for secondary sequence data analysis, such as DNA sequencing analysis or RNA sequencing analysis. In some embodiments, the secondary sequence data analysis comprises demultiplexing, trimming, read alignment, and UMI reference building. Non-limiting examples of programs or software include for performing secondary sequence data analysis include, but are not limited to Velvet, DRAGEN BioIT (Illumina®), SMRT (PacBio), MinKNOW and EPI2ME (Oxford Nanopore), or Burrows-Wheeler Alignment based algorithms (e.g., bowtie and SOAP2).

The CPU can be part of a circuit, such as an integrated circuit. One or more other components of the system can be included in the circuit. In some cases, the circuit is an application specific integrated circuit (ASIC).

The storage unit can store files, such as drivers, libraries and saved programs. The storage unit can store user data, e.g., user preferences and user programs. The computer system in some cases can include one or more additional data storage units that are external to the computer system, such as located on a remote server that is in communication with the computer system through an intranet or the Internet.

The computer system can communicate with one or more remote computer systems through the network. For instance, the computer system can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system via the network.

### II. METHODS

Disclosed herein, in some embodiments, are methods of utilizing the systems disclosed herein to identify bioactive molecules that modulate activity of a target enzyme (e.g., protease, phosphatase) or metabolic pathways that produce intermediates for producing the bioactive molecules, or both. The methods disclosed herein may be modified by applying molecular barcodes to the nucleic acid molecules encoded by the two-hybrid system or the metabolic system that can be used to demultiplex samples from multiplex sequencing analysis.

Provided herein are methods for performing multiplexed discovery of bioactive molecules that inhibit activity of a target enzyme, the method comprising: (a) providing a plurality of cells; (a) introducing into each of the plurality of cells a genetically-encoded system that links expression of a gene of interest to biosynthesis of a bioactive molecule by a cell of the plurality of cells, wherein the genetically-encoded system encodes the target enzyme, a synthase of the bioactive molecule, a ligand, and a receptor specific to the ligand under conditions sufficient for binding of the ligand to the receptor to produce a ligand-receptor pair, wherein the ligand-receptor pair induces expression of the gene of interest that is increased relative to a reference expression level when the bioactive molecule that inhibits the activity of the target enzyme is present in the cell, wherein the binding does not induce the expression of the gene of interest that is increased relative to the reference expression level when the bioactive molecule that inhibits the activity of the target enzyme is not present in the cell; (b) measuring the expression of the gene of interest that is increased relative to the reference expression level in a subset of the plurality of cells; and (c) performing multiplexed sequencing of the subset of the plurality of cells to discover the bioactive molecules that inhibit the activity of the target enzyme produced by the subset of the plurality of cells. In some embodiments, the reference expression level is obtained from an otherwise identical reference cell that does not comprise a functional version of the synthase, the ligand or the receptor. In some embodiments, the target enzyme comprises a proteolytic enzyme or a phosphatase. In some embodiments, the phosphatase comprises a tyrosine phosphatase. In some embodiments, inhibition of the target enzyme by the bioactive molecule disrupts binding between the receptor and the ligand, thereby preventing the ligand-receptor pair from inducing expression of the gene of interest. In some embodiments, the binding of the ligand to the receptor is phosphorylation dependent. In some embodiments, the synthase comprises a terpene synthase or a nonribosomal peptide synthetase. In some embodiments, the terpene synthase comprises γ-humulene synthase (GHS), amorphadiene synthase (ADS), α-bisabolene synthase (ABS), or taxadiene synthase (TXS). In some embodiments, the terpene synthase comprises an amino acid sequence that is greater than or equal to about 90% identical to any one of SEQ ID NO: 4, 7, 13, or 17. In some embodiments, the ligand comprises a kinase substrate that binds to the receptor in a phosphorylated state, and the receptor comprises a phosphorylated protein binding domain that binds to the kinase substrate when the kinase substrate is phosphorylated. In some embodiments, the ligand is coupled to an omega subunit of RNA polymerase and the receptor is coupled to a DNA binding domain, or the ligand is coupled to the DNA binding domain and the receptor is coupled to the omega subunit of the RNA polymerase. In some embodiments, the gene of interest encodes a reporter polypeptide comprising a luciferase enzyme, a fluorescent polypeptide, secreted alkaline phosphatase, ß-galactosidase levansucrase, chloramphenicol acetyltransferase (CAT), or antibiotic resistance. In some embodiments, the gene of interest encodes a polymerizing enzyme that, when expressed, binds to a promoter operably linked to a gene encoding the reporter polypeptide to drive expression of the reporter polypeptide. In some embodiments, the genetically-encoded system further encodes a metabolic pathway for biosynthesis of the bioactive molecule. In some embodiments, metabolic pathway is an isoprenoid pathway. In some embodiments, the isoprenoid pathway comprises a mevalonate pathway, a methylerthritol 4-phosphate (MEP) pathway, or a deoxyxylulose 5-phosphate (DXP) pathway. In some embodiments, the multiplex sequencing comprises long read sequencing. In some embodiments, the exogenous genetically-encoded system comprises one or more molecular barcode sequences that uniquely identifies the target enzyme, the synthase, or a combination thereof. In some embodiments, the multiplex sequencing further comprises performing demultiplexing, thereby assigning each of the one or more molecular barcodes with the target enzyme, the synthase, or the combination thereof, for each the subset of the plurality of cells.

Provided herein are methods of identifying a bioactive molecule that modulates a target enzyme disclosed herein. In some embodiments, the target enzyme is a protease or a phosphatase (e.g., tyrosine phosphatase). In some embodiments, the bioactive molecule is an inhibitor of the target enzyme. In some embodiments, the methods disclosed herein for identifying a bioactive molecule that inhibits a target enzyme, in some embodiments, comprise: (a) expressing in a cell an exogenous synthase for producing the bioactive molecule in the cell; (b) expressing in the cell a metabolic pathway under conditions suitable to provide metabolic intermediates for producing the bioactive molecule by the exogenous synthase; (c) introducing into the cell a two-hybrid system disclosed herein that links modulation of the target enzyme with expression of a gene of interest (GOI); and (d) measuring expression of the GOI. In some embodiments the exogenous synthase comprises a terpene synthase. In some embodiments, the target enzyme comprises a protease. In some embodiments, the target enzyme comprises a phosphatase. In some embodiments, the phosphatase is a tyrosine phosphatase. In some embodiments, the metabolic pathway comprises enzymes, metabolites, and/or intermediates of a mevalonate pathway, a methylerthritol 4-phosphate (MEP) pathway, a deoxyxylulose 5-phosphate (DXP) pathway, or a combination thereof. In some embodiments, the metabolic pathway results in isopentenyl diphosphate (IPP), or dimethylallyl diphosphate (DMAPP), or a combination thereof. In some embodiments, an increased expression of the gene of interest (GOI) as compared to a reference expression level indicates a presence of the inhibitor of the target enzyme produced by the cell. In some embodiments, a decreased expression of the GOI as compared to a reference expression level indicates an absence of an inhibitor of the target enzyme produced by the cell. In some embodiments, the reference expression level may be derived from a reference cell expressing a modified phosphatase/kinase substrate (e.g., MidT) containing a mutation that inhibits its binding to the phosphorylated protein binding domain (e.g., SH2). In some embodiments, the reference expression level may be derived from a reference cell expressing a modified synthase that contains a mutation that reduces the activity of the synthase. In some embodiments, the method further comprises comparing cell survival or growth, cell size, fluorescence, luminescence, or light absorption between the reference cell and a cell disclosed herein. In some embodiments, the method comprises repeating (a) to (d), wherein for each repetition, a new exogenous synthase may be used to identify a new bioactive molecule of the target enzyme. In some embodiments, the inhibitor of the target protease may be a terpene or terpenoid.

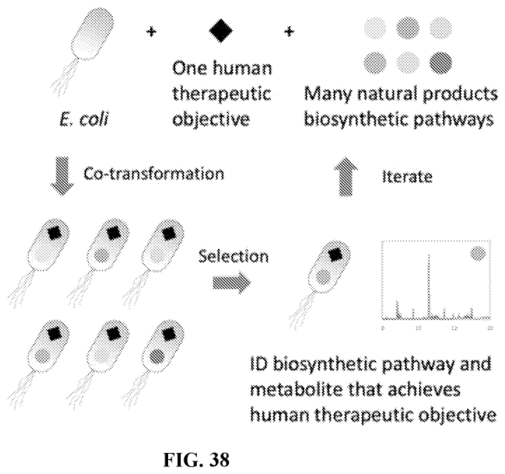

Also provided are methods of identifying metabolic pathways that produce bioactive molecules that modulate target enzymes. Referring to FIG. 38, a cell (e.g., a microbial cell) may be encoded with a bacterial two-hybrid system that links the modulation of a target enzyme to the expression of a gene of interest (GOI), and this encoded cell may be additionally transformed with a large library of biosynthetic pathways such that each transformed cell has a different pathway or set of pathways, and GOI expression enables the identification of the subset of pathways that produce a small-molecule that modulates target enzyme activity.

Provided herein are methods of expressing one or more heterologous nucleic acid molecules in a cell. In some embodiments, the heterologous nucleic acid may be introduced into the cell by transfection, transduction, or other suitable method. Suitable methods may be found in Chong Z X, Yeap S K, Ho W Y. Transfection types, methods and strategies: a technical review. Peer J. 2021 Apr. 21; 9:e11165, which is incorporated by reference in its entirety. In some embodiments, the transfection is transient. In some embodiments, the one or more heterologous nucleic acid molecules are comprised in one or more plasmid vectors. In some embodiments, the transfection is performed using electroporation, injection, nucleofection, sonoporation, magnetofection, or using a laser beam. In some embodiments, the transfection is performed using chemical to aid in the transfection, such as for example, lipid-based transfection. In some embodiments, another chemical approach is used, such as for example, using micro-/nano-particles, polymers, peptides/cations, calcium phosphate or dendrimers. In some embodiments, the one or more heterologous nucleic acid molecules may be introduced to the cell by transduction. In some embodiments, the one or more heterologous nucleic acid molecules are comprised in one or more viral vectors. In some embodiments, the transduction is transient. In some embodiments, the transient transduction may be performed using adenovirus, adeno-associated virus, lentivirus, or Herpes virus mediated transduction.

In some embodiments, methods comprise providing a cell described herein, and introducing one or more heterologous nucleic acid molecules encoding a synthase disclosed herein. In some embodiments, the synthase is a terpene synthase. In some embodiments, the terpene synthase is a modified terpene synthase relative to a wild-type terpene synthase. In some embodiments, the cell is a microbial cell, such as a bacterial cell (e.g., E. coli). In some embodiments, cell had been previously engineered to expresses the metabolic pathway under conditions suitable to provide metabolic intermediates for producing the bioactive molecule by the exogenous synthase. In other embodiments, the methods further comprise introducing one or more heterologous nucleic acid molecules encoding the metabolic pathway disclosed herein. In some embodiments, the methods further comprise introducing one or more heterologous nucleic acid molecules encoding the two-hybrid system disclosed herein. In some embodiments, the method of introducing the two-hybrid system into the cell is performed under conditions sufficient to cause the RNA polymerase omega subunit to recruit RNA polymerase to the binding site for RNA polymerase in the absence of a target enzyme (e.g., protease, phosphatase) inhibitor, thereby expressing the reporter gene.

In some embodiments, methods further comprise culturing the cell in a growth cell medium. In some embodiments the growth cell medium may comprise glycerol at a concentration between 0% and 2% (by volume). In some embodiments, the growth medium comprises mevalonate at a concentration between 0 mM and 20 mM. In some embodiments, the growth medium comprises iPTG at a concentration between 0 mM and 0.5 mM. In some embodiments, the growth medium comprises MOPS at a concentration between 0 mM and 50 mM. In some embodiments, the growth medium comprises sucrose at a concentration between 0% and 5% weight/volume.

In some embodiments, the cell is incubated for a certain length of time. In some embodiments, the length of time comprises more than 10 seconds and no more than 4 weeks, 1-10 minutes, 1-60 minutes, 0-24 hours, 0-48 hours, 0-72 hours, 1-5 days, 1-7 days, 0-4 weeks, or 1-4 weeks. In some embodiments, the length of time comprises about 10 seconds, 11 seconds, 12 seconds, 13 seconds, 14 seconds, 15 seconds, 16 seconds, 17 seconds, 18 seconds, 19 seconds, 20 seconds, 21 seconds, 22 seconds, 23 seconds, 24 seconds, 25 seconds, 26 seconds, 27 seconds, 28 seconds, 29 seconds, 30 seconds, 31 seconds, 32 seconds, 33 seconds, 34 seconds, 35 seconds, 36 seconds, 37 seconds, 38 seconds, 39 seconds, 40 seconds, 41 seconds, 42 seconds, 43 seconds, 44 seconds, 45 seconds, 46 seconds, 47 seconds, 48 seconds, 49 seconds, 50 seconds, 51 seconds, 52 seconds, 53 seconds, 54 seconds, 55 seconds, 56 seconds, 57 seconds, 58 seconds, 59 seconds, 60 seconds, 2 minutes, 3 minutes, 5 minutes, 6 minutes, 7 minutes, 8 minutes, 9 minutes, 10 minutes, 15 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 24 hours, 36 hours, 48 hours, 56 hours, 72 hours, 96 hours, 120 hours, 4 days, 5 days, 6 days, 7 days, 8 days, 10 days, 11 days, 12 days 13 days, 2 weeks, 3 weeks, or 4 weeks. In some embodiments, the cell is incubated at a temperature comprising no lower than 4 degrees Celsius and no higher than 40 degrees Celsius. In some embodiments, the cell is incubated at a temperature comprising about 4-40 degrees Celsius, 4-37 degrees Celsius, 20-40 degrees Celsius, 20-37 degrees Celsius, 20-30 degrees Celsius, 20-25 degrees Celsius, 30-35 degrees Celsius, or 25-37 degrees Celsius. In some embodiments, the cell is incubated at a temperature comprising about 4 degrees Celsius, 20 degrees Celsius, 21 degrees Celsius, 22 degrees Celsius, 23 degrees Celsius, 24 degrees Celsius, 25 degrees Celsius, 26 degrees Celsius, 27 degrees Celsius, 28 degrees Celsius, 29 degrees Celsius, 30 degrees Celsius, 31 degrees Celsius, 32 degrees Celsius, 33 degrees Celsius, 34 degrees Celsius, 35 degrees Celsius, 36 degrees Celsius, 37 degrees Celsius, 38 degrees Celsius, 39 degrees Celsius, or 40 degrees Celsius. In some embodiments, the cell is cultured in a suspension. In some embodiments, the cell is cultured in a solid medium, such as an agarose plate.

In some embodiments, the methods further comprise selecting the cell colonies containing the modulator of the target enzyme for further analysis by identifying the colonies that express the GOI. In some embodiments, where the GOI encodes for antibiotic resistance, the cells are plated and incubated on a solid medium containing an antibiotic (e.g., kanamycin, tetracycline, chloramphenicol) that is lethal to the cells that do not express the GOI. In some embodiments, where the GOI encodes an enzyme that produces a luminescent biomolecule (e.g., LuxAB), the cell colonies are expanded on solid medium, and introduced to a substrate of the enzyme, and cell colonies that produce the luminescent biomolecule are visible. In some embodiments, where the GOI encodes a fluorescent biomolecule (e.g., GFP) directly or indirectly, the cell colonies comprising the fluorescent biomolecule are visible. In some embodiments, the signal produced from the fluorescent biomolecule is increased by 2-fold, 3-fold, 4-fold, 5-fold, 10-fold, or 100-fold when it is expressed indirectly (e.g., when the GOI encodes an RNA polymerase that induced expression of the fluorescent biomolecule) than if expressly directly from the GOI. In some embodiments, the signal produced from the fluorescent biomolecule is increased by 2-fold to 100-fold, 3-fold to 90-fold, 4-fold to 80-fold, 5-fold to 70-fold, 6-fold to 60-fold, 7-fold to 50-fold, 8-fold to 40-fold, 9-fold to 30-fold, 10-fold to 20-fold when it is expressly indirectly rather than directly from the GOI. In some embodiments, the cell colonies that express the modulator of the target enzyme are cultured in suspension and expanded until they reach a certain optical density (OD) of about 600. In some embodiments, the cells are isolated from the liquid medium and pelleted using centrifugation, and stored as necessary before further analysis.

Provided herein are methods of determining a presence of a bioactive molecule that inhibits activity of a target enzyme, the method comprising: (a) introducing into a cell an exogenous genetically-encoded system that links expression of a gene of interest to biosynthesis of the bioactive molecule by the cell, wherein the exogenous genetically-encoded system encodes the target enzyme, a synthase of the bioactive molecule, a ligand and a receptor specific to the ligand under conditions sufficient for binding of the ligand to the receptor to form a ligand-receptor pair, wherein the ligand-receptor pair induces expression of the gene of interest that is increased relative to a reference expression level when the bioactive molecule that inhibits the activity of the target enzyme is present in the cell, wherein the binding does not induce the expression of the gene of interest that is increased relative to the reference expression level when the bioactive molecule that inhibits the activity of the target enzyme is not present in the cell; (b) measuring the expression of the reporter polypeptide; and (c) determining the presence of the bioactive molecule in the cell if the expression of the gene of interest is increased relative to the reference expression level. In some embodiments, the reference expression level is obtained from an otherwise identical reference cell that does not comprise a functional version of the synthase, the ligand or the receptor. In some embodiments, inhibition of the target enzyme by the bioactive molecule disrupts binding between the receptor and the ligand, thereby preventing the ligand-receptor pair from inducing expression of the gene of interest. In some embodiments, the binding of the ligand to the receptor is phosphorylation dependent. In some embodiments, the synthase comprises a terpene synthase or a nonribosomal peptide synthetase. In some embodiments, the terpene synthase comprises γ-humulene synthase (GHS), amorphadiene synthase (ADS), α-bisabolene synthase (ABS), or taxadiene synthase (TXS). In some embodiments, the terpene synthase comprises an amino acid sequence that is greater than or equal to about 90% identical to any one of SEQ ID NO: 4, 7, 13, or 17. In some embodiments, the terpene synthase is a catalytically active portion thereof. In some embodiments, the catalytically active portion of the terpene synthase comprises an amino acid sequence that is greater than or equal to about 90% identical to any one of the amino acid sequences provided in Table 30. In some embodiments, the ligand comprises a kinase substrate that binds to the receptor in a phosphorylated state, and the receptor comprises a phosphorylated protein binding domain that binds to the kinase substrate when the kinase substrate is phosphorylated. In some embodiments, the ligand is coupled to an omega subunit of RNA polymerase or portions thereof and the receptor is coupled to a DNA binding domain, or the ligand is coupled to the DNA binding domain and the receptor is coupled to the omega subunit or portions thereof of the RNA polymerase. In some embodiments, the gene of interest encodes a reporter polypeptide comprising a luciferase enzyme, a fluorescent polypeptide, secreted alkaline phosphatase, ß-galactosidase levansucrase, chloramphenicol acetyltransferase (CAT), or antibiotic resistance. In some embodiments, the gene of interest encodes a polymerizing enzyme that, when expressed, binds to a promoter operably linked to a gene encoding the reporter polypeptide to drive expression of the reporter polypeptide. In some embodiments, the genetically-encoded system further encodes a metabolic pathway for biosynthesis of the bioactive molecule. In some embodiments, metabolic pathway is an isoprenoid pathway. In some embodiments, the isoprenoid pathway comprises a mevalonate pathway, a methylerthritol 4-phosphate (MEP) pathway, or a deoxyxylulose 5-phosphate (DXP) pathway. In some embodiments, the signal produced from the reporter polypeptide is increased by 2-fold, 3-fold, 4-fold, 5-fold, 10-fold, or 100-fold when it is expressed indirectly (e.g., when the GOI encodes an RNA polymerase that induced expression of the reporter polypeptide) than if expressly directly from the GOI. In some embodiments, the signal produced from the fluorescent biomolecule is increased by 2-fold to 100-fold, 3-fold to 90-fold, 4-fold to 80-fold, 5-fold to 70-fold, 6-fold to 60-fold, 7-fold to 50-fold, 8-fold to 40-fold, 9-fold to 30-fold, 10-fold to 20-fold when it is expressly indirectly rather than directly from the GOI.

In some embodiments, the method may further comprise measuring the expression of said reporter polypeptide comprising a protein that confers antibiotic resistance by using drops of liquid culture to seed cells on solid media containing different concentrations of antibiotic such that cells that produce a bioactive molecule that modulates the activity of said target enzyme grow to higher concentrations of antibiotic than cells that do not produce that molecule or that produce less of it.

In some embodiments, the method may further comprise isolating a bioactive molecule that modulates the target enzyme (e.g., inhibitor of the protease or phosphatase). In some embodiments, the target enzyme comprises a target phosphatase disclosed herein In some embodiments, the target enzyme comprises a target protease. In some embodiments, the target protease may comprise a viral protease. In some embodiments, the viral protease may comprise HIV-1 protease (HIV-1Pr) or SARS-COV-2 main protease (3ClPro). Methods of isolating the bioactive molecule comprises (1) breaking the cells to release their chemical constituents; (2) extracting the sample using a suitable solvent (or through distillation or the trapping of compounds); (3) separating the desired bioactive molecule (e.g., terpene or terpenoid) from other undesired contents of the extracts that confound analysis and quantification; and (4) use an appropriate method of analysis (e.g. thin layer chromatography [TLC], gas chromatography [GC], or liquid chromatography [LC]), as discussed in Jiang Z, Kempinski C, Chappell J. “Extraction and Analysis of Terpenes/Terpenoids.” Curr Protoc Plant Biol. 1 (2016) 345-358, which is hereby incorporated by reference in its entirety. In some embodiments, Nuclear Magnetic Resonance (NMR) is also used to examine molecules. In some embodiments, only the cell cultures are spun down and only the culture supernatant is analyzed. In some embodiments, the cells are spun down, washed, and lysed, and only the intracellular molecules are analyzed. In some embodiments, both extracellular and intracellular molecules are analyzed.

Provided herein, are high throughput methods of identifying a plurality of modulators of a plurality of target enzymes using multiplex sequencing analysis. In some embodiments, the method may comprise: (a) introducing into a plurality of cells a nucleic acid sequence encoding exogenous synthases for producing the bioactive molecule in the cell; (b) introducing into the plurality of cells one or more nucleic acid sequences encoding a metabolic pathway under conditions suitable to provide metabolic intermediates for producing the bioactive molecule by the exogenous synthases; (c) introducing into the plurality of cells two-hybrid system that links the modulation of a target enzyme of the plurality of target enzymes with expression of a GOI, wherein the two-hybrid system comprises a nucleic acid sequence encoding a target enzyme, wherein the nucleic acid sequence comprises one or more molecular barcodes corresponding to the target enzyme, the metabolic pathway, the synthase, or a combination thereof; (d) measuring expression of the reporter gene in the plurality of cells; (e) performing multiplexed sequencing analysis to produce sequencing data; (f) demultiplexing the sequencing data by assigning the one or more molecular barcodes of each of the plurality of cells to the respective target enzyme produced by that cell. In some embodiments, the nucleic acid sequence encoding each of the exogenous synthases is also barcoded to enable to assignment of the synthase (or mutant thereof) and the resulting bioactive molecule produced by the cell. In some embodiments, methods further comprise detecting an increased expression of the GOI in a subset of the plurality of cells, as compared to a reference expression level, which is thereby indicative that the subset of the plurality of cells produced an inhibitor of the target enzyme. In some embodiments, multiplexed sequencing analysis is performed on the plurality of cells prior to (d) to measure baseline expression of a terpene synthase in each of the plurality of cells. In some embodiments, the multiplex sequencing in (e) is performed on a subset of the plurality of cells with an increase or a decrease in the expression for the reporter gene (e.g., indicating presence of a bioactive molecule modulating the activity of the target enzyme) to measure the expression of a terpene synthase. In some embodiments, enrichment of the terpene synthase is determined by comparing the expression of the terpene synthase to identify the subset of the plurality of cells that produced a bioactive molecule modulating the activity of the target enzyme.

Provided herein, are high throughput methods of identifying a plurality of modulators of a plurality of target enzymes using multiplex sequencing analysis. In some embodiments, the method may comprise: (a) introducing into a plurality of cells a nucleic acid sequence encoding exogenous synthases for producing the bioactive molecule in the cell; (b) introducing into the plurality of cells one or more nucleic acid sequences encoding a metabolic pathway under conditions suitable to provide metabolic intermediates for producing the bioactive molecule by the exogenous synthases, wherein the one or more nucleic acid sequences encoding the metabolic pathway comprises one or more molecular barcodes corresponding to the metabolic pathway; (c) introducing into the plurality of cells two-hybrid system that links the inhibition of a target enzyme of the plurality of target enzymes with expression of a GOI, wherein the two-hybrid system comprises a nucleic acid sequence encoding a target enzyme; (d) measuring expression of the reporter gene in the plurality of cells; (e) using multiplexed sequencing analysis to produce sequencing data; (f) demultiplexing the sequencing data by assigning the one or more molecular barcodes of each of the plurality of cells to the respective target enzyme produced by that cell. In some embodiments, the nucleic acid sequence encoding each of the exogenous synthases is also barcoded to enable to assignment of the synthase (or mutant thereof) and the resulting bioactive molecule produced by the cell. In some embodiments, methods further comprise detecting an increased expression of the GOI in a subset of the plurality of cells, as compared to a reference expression level, which is thereby indicative that the subset of the plurality of cells produced an inhibitor of the target enzyme. In some embodiments, the nucleic acid sequence encoding each of the target enzymes comprises a unique molecular barcode enabling the identification of the target enzyme with the bioactive molecule that is identified. In some embodiments, multiplexed sequencing analysis is performed on the plurality of cells prior to (d) to measure baseline expression of a terpene synthase in each of the plurality of cells. In some embodiments, the multiplex sequencing in (e) is performed on a subset of the plurality of cells with an increase or a decrease in the expression for the reporter gene (e.g., indicating presence of a bioactive molecule modulating the activity of the target enzyme) to measure the expression of a terpene synthase. In some embodiments, enrichment of the terpene synthase is determined by comparing the expression of the terpene synthase to identify the subset of the plurality of cells that produced a bioactive molecule modulating the activity of the target enzyme.

In some embodiments, the plurality of cells are pooled prior to measuring in (d), thereby reducing the time to perform the analysis. In this manner, from 10² to 10¹⁰ colony-forming cells may be analyzed in parallel, thereby drastically reducing the time of analysis for large screens. In some embodiments, multiple bioactive molecules may be identified in a single implementation of the method. In some embodiments, from 10² to 10¹⁰, 10³ to 10⁹, 10⁴ to 10⁸, 10⁵ to 10⁷ colony-forming cells may be analyzed in parallel. In some embodiments, more than or equal to about 10², 10³, 10⁴, 10⁵, 10⁶, 10⁷, 10⁸, 10⁹, or 10¹⁰ colony-forming cells may be analyzed in parallel. In some embodiments, fewer than or equal to about 10², 10³, 10⁴, 10⁵, 10⁶, 10⁷, 10⁸, 10⁹, or 10¹⁰ colony-forming cells may be analyzed in parallel.

In some embodiments, the multiplex sequencing comprises sequencing-by-synthesis, sequencing by transient binding, single-molecule real-time sequencing, ion semiconductor sequencing (Iron Torrent®), pyrosequencing, combinatorial probe anchor synthesis (cPAS), sequencing-by-ligation, nanopore sequencing, or semiconductor-based electronic sequencing (GenapSys™). In some embodiments, the assessing the enrichment of target enzymes within the subset of cells, as compared to the plurality of cells, is performed by a computer processor programmed to demultiplex the genetic information that was sequenced. In some embodiments, the primary or secondary sequencing data analysis is performed by a computer systems disclosed herein. In some embodiments, the secondary sequence data analysis comprises demultiplexing the molecular barcodes sufficient to identify the metabolic pathway, the synthase, or both that produced the bioactive molecule with therapeutic potential, or the target enzyme that the bioactive molecule inhibits, or a combination thereof.

In some embodiments, methods further comprise introducing into each cell of the plurality of cells a nucleic acid sequence encoding the unique terpene synthase. In some embodiments, the nucleic acid sequence comprises a barcode sufficient to identify the terpene synthase. In some embodiments, the method may further comprise: (a) identifying the second barcode in cells within each of (i) the plurality of cells and (ii) a subset of the plurality of cells with an increased expression level of the reporter gene, (b) assessing the enrichment of terpene synthases within the subset of cells, as compared to the plurality of cells, thereby identifying which of the unique exogenous terpene synthase in each cell produces the inhibitor of the target enzyme in that cell. In some embodiments, the one or more nucleic acid sequence encoding the metabolic pathway encodes an enzyme that catalyzes the condensation of isopentenyl diphosphate (IPP), dimethylallyl diphosphate (DMAPP), or a combination of IPP and DMAPP. In some embodiments, the enzyme comprises geranylgeranyl diphosphate synthase (GGPPS). In some embodiments, the one or more nucleic acid sequences further comprises a cytochrome P450 enzyme, a cytochrome P450 reductase enzyme, a cytochrome b5 enzyme, an oxidase enzyme, an acyl transferase enzyme, a glycosyltransferase enzyme, a halogenase, or a peroxidase, or a combination thereof. In some embodiments, the one or more nucleic acid sequences encode a metabolic pathway for IPP, DMAPP and/or molecules resulting from the condensation of IPP and/or DMAPP. In some embodiments, the GOI encodes a polymerizing enzyme that, when expressed, binds to a promoter operably linked to a gene encoding a detectable polypeptide to drive expression of the detectable polypeptide, wherein the detectable polypeptide optionally may comprise a fluorescent polypeptide. In some embodiments, the expression of the detectable polypeptide may be greater than an expression of the detectable polypeptide when its gene may be included as the reporter gene.

Also provided are methods of adding one or more molecular barcodes to one or more nucleic acids disclosed herein. In some embodiments, the one or more barcodes may be added by polymerase chain reaction (PCR), ligation, or transposition. Suitable techniques for attaching molecular barcodes to one or more nucleic acids disclosed herein are provided in Head, Steven R., et al. “Library construction for next-generation sequencing: overviews and challenges.” Biotechniques 56.2 (2014): 61-77; and in Hu, Taishan, et al. “Next-generation sequencing technologies: An overview.” Human Immunology 82.11 (2021): 801-811; and in Gkazi, Athina. “An Overview of Next-Generation Sequencing.” (2021), which is hereby incorporated by reference in its entirety

In some embodiments, the method may further comprise culturing the plurality of cells in a growth cell medium, provided in Section I (A) (1) herein. In some embodiments, the growth cell medium comprises (i) glycerol at a concentration between 1 and 2%, (ii) mevalonate at a concentration between 0 and 20 mM, (iii) or a combination of (i) and (ii).

Also provided are methods of determining a presence of a bioactive molecule that inhibits activity of a target enzyme, the method comprising: (a) introducing into a cell an exogenous genetically-encoded system that links expression of a gene of interest to biosynthesis of the bioactive molecule by the cell, wherein the exogenous genetically-encoded system encodes the target enzyme, a synthase of the bioactive molecule, a ligand and a receptor specific to the ligand under conditions sufficient for binding of the ligand to the receptor to form a ligand-receptor pair, wherein the ligand-receptor pair comprises a cleavage site recognized by the proteolytic enzyme, and induces expression (e.g., activates transcription) of the gene of interest that is increased relative to a reference expression level when the bioactive molecule that inhibits the activity of the target enzyme is present in the cell, wherein the binding does not induce the expression of the gene of interest that is increased relative to the reference expression level when the bioactive molecule that inhibits the activity of the target enzyme is not present in the cell, wherein the target enzyme comprises a proteolytic enzyme; (b) measuring the expression of the gene of interest; and (c) determining the presence of the bioactive molecule in the cell if the expression of the gene of interest is increased relative to the reference expression level. In some embodiments, the reference expression level is obtained from an otherwise identical reference cell that does not comprise a functional version of the synthase, the ligand or the receptor. In some embodiments, inhibition of the target enzyme by the bioactive molecule disrupts binding between the receptor and the ligand, thereby preventing the ligand-receptor pair from inducing expression of the gene of interest. In some embodiments, the binding of the ligand to the receptor is phosphorylation dependent. In some embodiments, the synthase comprises a terpene synthase or a nonribosomal peptide synthetase. In some embodiments, the terpene synthase comprises γ-humulene synthase (GHS), amorphadiene synthase (ADS), α-bisabolene synthase (ABS), or taxadiene synthase (TXS). In some embodiments, the terpene synthase comprises an amino acid sequence that is greater than or equal to about 90% identical to any one of SEQ ID NO: 4, 7, 13, or 17. In some embodiments, the ligand comprises a kinase substrate that binds to the receptor in a phosphorylated state, and the receptor comprises a phosphorylated protein binding domain that binds to the kinase substrate when the kinase substrate is phosphorylated. In some embodiments, the ligand is coupled to an omega subunit of RNA polymerase or portions thereof and the receptor is coupled to a DNA binding domain, or the ligand is coupled to the DNA binding domain and the receptor is coupled to the omega subunit or portions thereof of the RNA polymerase. In some embodiments, the gene of interest encodes a reporter polypeptide comprising a luciferase enzyme, a fluorescent polypeptide, secreted alkaline phosphatase, ß-galactosidase levansucrase, chloramphenicol acetyltransferase (CAT), or antibiotic resistance. In some embodiments, the gene of interest encodes a polymerizing enzyme that, when expressed, binds to a promoter operably linked to a gene encoding the reporter polypeptide to drive expression of the reporter polypeptide. In some embodiments, the genetically-encoded system further encodes a metabolic pathway for biosynthesis of the bioactive molecule. In some embodiments, metabolic pathway is an isoprenoid pathway. In some embodiments, the isoprenoid pathway comprises a mevalonate pathway, a methylerthritol 4-phosphate (MEP) pathway, or a deoxyxylulose 5-phosphate (DXP) pathway. In some embodiments, the signal produced from the reporter polypeptide is increased by 2-fold, 3-fold, 4-fold, 5-fold, 10-fold, or 100-fold when it is expressed indirectly (e.g., when the GOI encodes an RNA polymerase that induced expression of the reporter polypeptide) than if expressly directly from the GOI. In some embodiments, the signal produced from the fluorescent biomolecule is increased by 2-fold to 100-fold, 3-fold to 90-fold, 4-fold to 80-fold, 5-fold to 70-fold, 6-fold to 60-fold, 7-fold to 50-fold, 8-fold to 40-fold, 9-fold to 30-fold, 10-fold to 20-fold when it is expressly indirectly rather than directly from the GOI.

### III. KITS

Provided herein, in some aspects are kits comprising the one or more system components disclosed herein. In some embodiments, the kits comprise one or more components of the genetically-encoded system described herein. In some embodiments, the kits comprise the one or more nucleic acid molecules encoding the two-hybrid system (e.g., B2H system). In some embodiments, the kits comprise the one or more nucleic acid molecules encoding the metabolic pathway. In some embodiments, the kits comprise the one or more nucleic acid molecules encoding the terpene synthase. In some embodiments, the kits further comprise a cell, or a plurality of cells. In some embodiments, the kits further comprise cell media, such as growth media. In some embodiments, the kits further comprise additional constituents of the cell media, such as mevalonate, antibiotics, and so forth. The exact nature of the components configured in the inventive kit depends on its intended purpose. For example, some kits are configured for the purpose of producing a genetically encoded microorganism, isolating a bioactive molecule from a plurality of genetically encoded microorganisms, or investigating therapeutic potential of the one or more bioactive molecules.

Instructions for use may be included in the kit. “Instructions for use” typically include a tangible expression describing the technique to be employed in using the components of the kit to effect a desired outcome, e.g., producing a genetically encoded microorganism, isolating a bioactive molecule from a plurality of genetically encoded microorganisms, or investigating therapeutic potential of the one or more bioactive molecules. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the user stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging materials employed in the kit are those customarily utilized in gene expression assays and in the administration of treatments. As used herein, the term “package” refers to a suitable solid matrix or material such as glass, plastic, paper, foil, and the like, capable of holding the individual kit components. Thus, for example, a package can be a plastic vial or tube used to contain suitable quantities of the genetically-encoded system, and/or cells. The packaging material generally has an external label which indicates the contents and/or purpose of the kit and/or its components.

### IV. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

The term “about” is used herein with reference to a number refers to that number plus or minus 10% of that number. The term “about” a range refers to that range minus 10% of its lowest value and plus 10% of its greatest value.

The terms, “polynucleotide,” or “nucleic acid,” are used interchangeably herein to refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as, but not limited to methylated nucleotides and their analogs or non-nucleotide components. Modifications to the nucleotide structure may be imparted before or after assembly of the polymer. A polynucleotide may be further modified after polymerization, such as by conjugation with a labeling component.

The term “cell,” as used herein, generally refers to a biological cell.

The term “gene,” as used herein, refers to a segment of nucleic acid that encodes an individual protein or RNA (also referred to as a “coding sequence” or “coding region”), optionally together with associated regulatory region such as promoter, operator, terminator and the like, which may be located upstream or downstream of the coding sequence. A “genetic locus” referred to herein, is a particular location within a gene.

The terms “increased” or “increase” are used herein to generally mean an increase by a statically significant amount.

The terms “decreased” or “decrease” are used herein generally to mean a decrease by a statistically significant amount.

The terms “polypeptide,” “peptide” and “protein” may be used interchangeably herein in reference to a polymer of amino acid residues. A protein may refer to a full-length polypeptide as translated from a coding open reading frame, or as processed to its mature form, while a polypeptide or peptide may refer to a degradation fragment or a processing fragment of a protein that nonetheless uniquely or identifiably maps to a particular protein. A polypeptide may be a single linear polymer chain of amino acids bonded together by peptide bonds between the carboxyl and amino groups of adjacent amino acid residues. Polypeptides may be modified, for example, by the addition of carbohydrate, phosphorylation, etc.

The terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87:2264-2268, 1990, modified as in Proc. Natl. Acad. Sci. USA 90:5873-5877, 1993). Such a formula is incorporated into the basic local alignment search tool (BLAST) programs of Altschul et al. (J Mol Biol. 1990 Oct. 5; 215 (3): 403-10; Nucleic Acids Res. 1997 Sep. 1; 25 (17): 3389-402). Percent homology of sequences can be determined using the most recent version of BLAST, as of the filing date of this application. Percent identity of sequences can be determined using the most recent version of BLAST, as of the filing date of this application.

The term “percent (%) identity”, or “percent sequence identity,” with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. As used herein, the term “percent (%) identity”, or “percent sequence identity,” with respect to a reference nucleic acid sequence is the percentage of nucleotides in a candidate sequence that are identical with the nucleotides in the reference nucleic acid sequence, after aligning the sequences and introducing gaps, if necessary, to achieve the maximum percent sequence identity. Alignment for purposes of determining percent sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

The term “two-hybrid system”, as used herein, refers to a genetic system for identifying protein-protein interactions (PPIs) and protein-DNA interactions. In some embodiments, the two-hybrid system detects interactions between the target enzyme and the target enzyme substrate by measuring activity of the target enzyme on the substrate evidenced by a readout of the genetic system, such as fluorescence or cell survival.

The terms “gene of interest” or “GOI,” as used interchangeably herein, refer to a gene encoding a gene expression product that is detectable directly or indirectly.

Amino acids disclosed herein may be represented by a one letter or three letter code under the Internal Union of Pure and Applied Chemistry (IUPAC) naming convention, as set forth in Table 23A.

TABLE 23A
IUPAC Amino Acid Code

| IUPAC | Three | |
|---|---|---|
| amino | letter | Amino |
| acid code | code | acid |
| A | Ala | Alanine |
| C | Cys | Cysteine |
| D | Asp | Aspartic Acid |
| E | Glu | Glutamic Acid |
| F | Phe | Phenylalanine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Tryptophan |
| Y | Tyr | Tyrosine |

A nucleotide disclosed herein may be represented by a one letter or symbol under the IUPAC naming convention, as set forth in Tables 23B below.

TABLE 23B
IUPAC Nucleotide Code

| IUPAC nucleotide code | Base |
|---|---|
| A | Adenine |
| C | Cytosine |
| G | Guanine |
| T (or U) | Thymine (or Uracil) |
| R | A or G |
| Y | C or T |
| S | G or C |
| W | A or T |
| K | G or T |
| M | A or C |
| B | C or G or T |
| D | A or G or T |
| H | A or C or T |
| V | A or C or G |
| N | any base |
| . or - | gap |

The term “bioactive molecule” as used herein refers to a molecule having a biologic effect on a living organism, tissue or cell.

The term, “metabolic pathway” as used herein refers to one or more chemical reactions carried out by constituents (e.g., reactants, products, intermediates) of the metabolic pathway within a cell. The phrase, “encoding a metabolic pathway” with reference to a nucleic acid molecule refers to a nucleic acid molecule encoding one or more of the constituents of the metabolic pathway. For instance, the metabolic pathway may be an isoprenoid pathway involved in the synthesis of isoprenoids. Non-limiting examples of isoprenoid pathways are mevalonate pathway and non-mevalonate pathway (e.g., methylerthritol 4-phosphate (MEP) or deoxyxylulose 5-phosphate (DXP) pathways). The isoprenoid pathway may produce isopentenyl diphosphate (IPP) or dimethylallyl diphosphate (DMAPP), which are precursors of isoprenoid biosynthesis. The metabolic pathway may be naturally occurring. The metabolic pathway may be synthetic. In either case, the metabolic pathway may be exogenous to the cell. A non-limiting example of a synthetic metabolic pathway is the isopentenol utilization pathway (IUP) described in AO Chatzivasileiou et al., “Two-step pathway for isoprenoid synthesis.” Applied Biological Sciences. 116 (2) 506-511 (Dec. 24, 2018), which is hereby incorporated by reference.

The term, “synthase” or “synthetase,” as used interchangeably herein, refers to an enzyme that is capable of catalyzing synthesis of a molecule. In some embodiments, the molecule is a bioactive molecule disclosed herein.

The term, “ligand” a used herein refers to molecule that binds to another molecule, such as a receptor. In some cases, the ligand binds to a receptor disclosed herein to serve a biological purpose, such as for example, activate transcription of a gene of interest (GOI). In some embodiments, the ligand is a phosphorylated amino acid.

The term, “receptor” as used herein refers to a protein that binds to a molecule, such as a ligand disclosed herein.

The term, “transcription” as used herein refers to the process by which the information in a strand of DNA is copied into a new molecule of messenger RNA (mRNA).

The term “polymerizing enzyme” as used herein refers to an enzyme or catalytically active portion thereof capable of polymerizing the synthesis of a polymer, such as a nucleic acid molecule. In some cases, the polymerizing enzyme is a “DNA polymerase,” which refers to an enzyme or catalytically active portion thereof capable of polymerizing the synthesis of DNA. In some cases, the polymerizing enzyme is an “RNA polymerase,” which refers to an enzyme or catalytically active portion thereof capable of polymerizing the synthesis of RNA.

The term “terpene” as used herein refers to an organic compound that is a simple hydrocarbon. In some cases, the terpene has 1, 2, 3, 4, 5, 6, 7, 8, or more isoprene units. Non-limiting examples of terpenes include isoprene, monoterpenes, sesquiterpenes, diterpenes, sesterterpenes, triterpenes, tetraterpenes and polyterpenes.

The term “terpenoid” or “isoprenoid” as used interchangeably herein refers to a terpene that has been modified to contain one or more functional groups, oxidized methyl groups, or a combination thereof. In some cases, the terpene has 1, 2, 3, 4, 5, 6, 7, 8, or more isoprene units. Non-limiting examples of terpenoids include hemiterpenoids, monoterpenoids, sesquiterpenoids, diterpenoids, sesterterpenoids, triterpenoids, tetraterpenoids, and polyterpenoids.

The term “isoprene” referred to herein, refers to 2-methyl-1,3-butadiene (e.g., CH2=C (CH3)-CH═CH2).

The term “isoprenoid” as used herein refers to an organic molecule containing two or more isoprene units.

The term “multiplex sequencing,” as used herein, refers to sequencing genetic information from two or more samples in a single sequencing run. In some cases, the two or more samples each comprise cells harboring a distinct two-hybrid system, a metabolic pathway, or both. In some embodiments, the multiplex sequencing includes pooling two or more samples prior to sequencing.

The term, “proteolytic enzyme” as used herein is an enzyme or catalytically active portion thereof capable of proteolysis. In some embodiments, the proteolytic enzyme is a protease, a peptidase, or proteinase. In some embodiments, the proteolytic enzyme is an exopeptidase or an endopeptidase.

The section headings used herein are for organizational purposes only and are not to be construed as limiting the subject matter described.

### V. EXAMPLES

The following examples are included for illustrative purposes only and are not intended to limit the scope of the inventive concepts.

### Example 1: Evolution-Guided Biosynthesis of Terpenoid Inhibitors

The molecules produced in the natural world have been shaped, over millennia, by enzymes evolving under selective pressure. Secondary metabolites—compounds that are not essential to the survival of their producers—are a remarkable outcome of this process; this class of chemicals encompasses an enormous diversity of molecular structures capable of carrying out complex biological functions. Many of the enzymes comprising secondary metabolic pathways are highly evolvable: mutations can alter their substrate specificity and reactivity to dramatically change the structures of the products they produce. This plasticity has been exploited to produce various compounds by engineering terpene synthases, the class of enzymes responsible for producing terpenoids (a vast natural product family including many secondary metabolites); however, systems that pair product diversification with a selective pressure to observe evolutionary trajectories of a terpene synthase are lacking. Here, a genetically encoded bacterial two-hybrid system conferring antibiotic resistance in response to the inactivation of heterologously expressed protein tyrosine phosphatase 1B (an important drug target) can be used to evolve a terpene synthase in E. coli. Starting with γ-humulene synthase, a low-producing terpene synthase generating many products, the work described herein shows that 1-2 mutations are sufficient to enhance resistance to an antibiotic in the system—an indication of drug target inhibition. The best mutants are better tolerated by E. coli and increase total terpene production, and one of these variants exhibits a product profile shifted towards two potential terpenoid inhibitors with titers increased 12- and 50-fold compared to the starting enzyme. The results demonstrate the feasibility of using genetically encoded selection pressures to evolve biosynthetic enzymes towards the production of biologically active molecules.

Terpenoids are the largest and most structurally diverse group of natural products and include a striking variety of biologically active compounds, from flavors to medicines. Terpenoids play an outsized role in the evolution and adaptation of living systems. These secondary metabolites carry out a broad set of physiological functions in their native hosts (e.g., signaling, protein localization, and protection from abiotic stress) and mediate essential interactions between unlike organisms (e.g., plants and pollinators, microbial pathogens, and symbionts). For millennia, their sophisticated biological activities have found use in flavors, fragrances, and medicines. Despite this well-documented biochemical versatility, the evolutionary processes that generate new functional terpenoids are poorly understood and difficult to recapitulate in engineered systems. This study uses a synthetic biochemical objective—a transcriptional system that links the inhibition of protein tyrosine phosphatase 1B (PTP1B), a human drug target, to the expression of a gene for antibiotic resistance in E. coli—to evolve γ-humulene synthase (GHS) to build terpenoid inhibitors. Site-saturation mutagenesis of poorly conserved residues yielded mutants that improved fitness (e.g., the antibiotic resistance of E. coli) by reducing GHS toxicity and/or by increasing inhibitor production. Intriguingly, a combination of two mutations enhanced the titer of a minority product—a terpene alcohol that inhibits PTP1β—by over fifty-fold, and a comparison of similar mutants enabled the identification of a site where mutations permit efficient hydroxylation. Findings illustrate how the plasticity of terpene synthases enables an efficient sampling of structurally distinct starting points for building new functional molecules and provide an experimental framework for exploiting this plasticity in activity-guided screens.

All natural terpenoids have a common biosynthetic origin. Their assembly begins with two C5 isoprenoid precursors—isopentenyl diphosphate (IPP) and dimethylallyl diphosphate (DMAPP)—which are synthesized from either (i) acetyl-CoA through the mevalonate pathway (MVA) or (ii) pyruvate and glyceraldehyde 3-phosphate through the non-mevalonate pathway (MEP or DXP). Condensation of IPP and DMAPP generates longer isoprenoids, such as geranyl diphosphate (GPP, C₁₀), farnesyl diphosphate (FPP, C₁₅), or geranylgeranyl diphosphate (GGPP, C₂₀), which are substrates for terpene synthases, P₄₅₀ monooxygenases, and acyltransferases. Metabolic engineers have resolved the biosynthetic pathways of many important terpenoids (e.g., artemisinin, paclitaxel, and momilactone B); the evolutionary transformations that allow them to build new functional molecules, however, are difficult to probe without a framework for carrying out biosynthesis under selective pressures.

FIGS. 1A-1D show (A) a promiscuous terpene synthase: γ-humulene synthase (GHS) that binds to farnesyl diphosphate (1), releases the terminal diphosphate, and cyclizes the resulting trans- or cis-farnesyl cation into over 50 terpenoid products, a subset of which appear here. Highlights show terpenoids generated from a shared intermediate. (B) A crystal structure of PTP1B bound to amorphadiene, an allosteric inhibitor (AD, pdb entry 6W30). An overlay of a competitive inhibitor (UN7) highlights the active site (aligned pdb entry 2F71). (C) Genetically encoded systems for (left) terpenoid biosynthesis and (right) inhibitor detection. Plasmids: pMBIS, the mevalonate-dependent isoprenoid pathway of S. cerevisiae; pTS, a terpene synthase; and pB2H, a two-hybrid system in which the inhibition of PTP1B permits a phosphorylation-mediated protein-protein interaction that activates transcription of a gene for spectinomycin resistance. Note: pMBIS and pTS can generate sesquiterpenes at titers of 0.36±0.05 mg/L (longifolene equivalents). (D) A selection scheme for identifying GHS mutants that generate PTP1B inhibitors.

Terpene synthases are centrally important to terpenoid diversity. These enzymes convert a few linear substrates into hundreds of complex scaffolds (e.g., hydrocarbons with multiple fused rings and stereocenters), which form the core of more than 95,000 known natural products. These enzymes are intriguing because they share a small set of domain architectures (a, αβ, βγ, or αβγ) and catalytic motifs (e.g., DDXDD and NSE for class I cyclases, and DXDD for class II cyclases), given their diverse product profiles. In general, terpene synthases can act on a small set of linear substrates by initiating a carbocation cyclization cascade and controlling it by constraining the conformational space and termination steps accessible to intermediates; as a result, mutations that affect the volume, contour, and solvation structure of the active site tend to alter product profiles. As a case study, γ-humulene synthase (GHS) from Abies grandis converts FPP into over 50 sesquiterpenes (FIG. 1), but a few amino acid substitutions can yield variants with 1-3 major products. Epi-isozizaene synthase from Streptomyces coelicolor generates a handful of sesquiterpenes, but the addition of a single polar residue to its nonpolar active site can yield mutants that produce entirely new molecules. The plasticity of terpene synthases—the sensitivity of their product profiles to a small number of mutations (e.g., amino acid substitutions)—enables rapid sampling of diverse structures and may facilitate the evolution of new functional molecules; however, the extent to which mutations in terpene synthases, alone, can improve fitness of living systems under shifting evolutionary constraints remains unclear.

In this study, E. coli was modified with a synthetic biochemical objective (the inhibition of protein tyrosine phosphatase 1B (PTP1B) from Homo sapiens) and was used to evolve mutants of GHS that achieve this objective. PTP1B is an influential regulatory enzyme, an important model system for biophysical studies, and an elusive drug target; new inhibitors could find broad use. GHS has a diverse, mutation-sensitive product profile and does not generate potent inhibitors of PTP1B in its wild-type form; it is a promising starting point for directed evolution. Using the artificial objective as a guide, the mutants of GHS were uncovered in which 1-2 amino acid substitutions confer a survival advantage by reducing GHS toxicity and/or by generating PTP1B inhibitors, which summarizing the mechanisms by which mutants enhance antibiotic resistance. Findings illustrated how terpene synthases can evolve quickly under artificial selection pressures to build biologically active molecules. The best performing mutants exhibited altered product profiles with a shared major product that inhibits PTP1B. These mutants illustrate how TSs can evolve with heterologous hosts to build molecules that solve new challenges.

To begin, a selective pressure was engineered to guide terpenoid biosynthesis in E. coli. A bacterial two-hybrid (B2H) system that links the inhibition of PTP1B to the expression of a gene for antibiotic resistance was specifically chosen. (FIGS. 1A-1D). In this system, Src kinase phosphorylates a substrate domain, allowing it to bind to a Src homology 2 (SH2) domain; the substrate-SH2 complex activates transcription of a resistance gene by localizing RNA polymerase to its promoter. PTP1B dephosphorylates the substrate domain, preventing transcription, and the inactivation of PTP1B reenables it. This system is capable of screening terpene synthases for their ability to generate inhibitors of PTP1B. Both amorphadiene (AD) synthase and α-bisabolene (AB) synthase confer a significant survival advantage (e.g., growth on solid media with ˜800 μg/ml spectinomycin—a concentration sufficient to kill some strains with inactive variants of both terpene synthases); subsequent kinetic and biophysical analyses suggested that AD inhibits PTP1B by binding to an allosteric site (FIGS. 1A-1D). The resistance conferred by GHS, by contrast, barely exceeded the threshold set by a negative control (˜200 μg/ml Spec).

Early studies of enzyme evolution used GHS as a model system. In one seminal study, researchers mutated 19 residues that line the active site of GHS and used the product profiles of single mutants to design variants with very narrow—and very different—product profiles. In a follow-up study, the researchers showed that the rational redistribution of glycine and proline residues (e.g., rational mutations informed by residue conservation in a multiple sequence alignment) could improve terpenoid production in E. coli. Both studies suggested that the effects of mutations were additive (e.g., substitutions that enhanced terpenoid production by GHS also did so for mutants with different product profiles). This work provided a powerful framework for the rational redesign of terpene synthases, but it did not attempt to evolve them under selective pressures. The present disclosure provides evolving these enzymes to produce molecules that address a genetically encoded challenge in a heterologous host. The B2H system provides an opportunity for such studies (FIGS. 1A-1D).

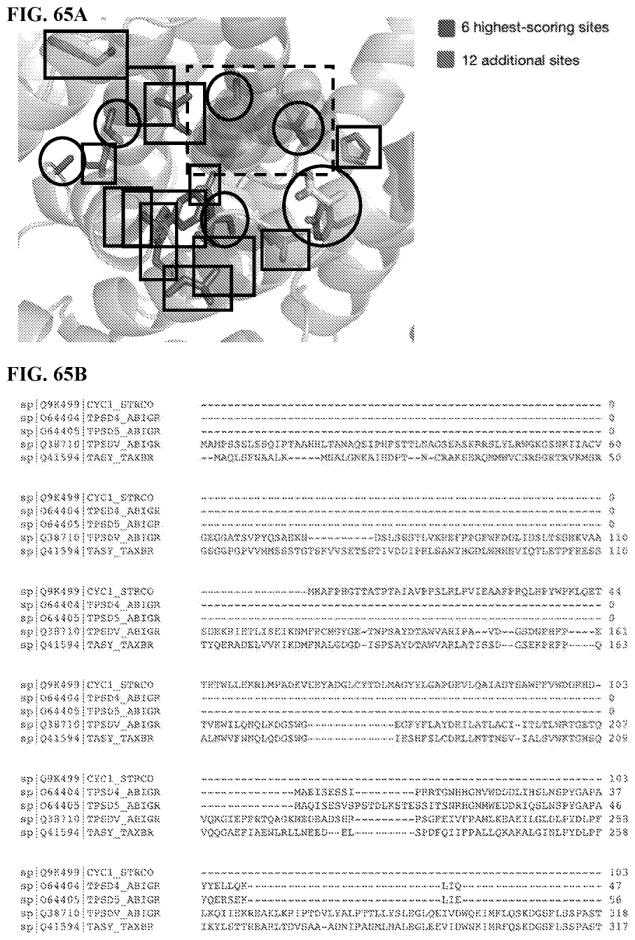

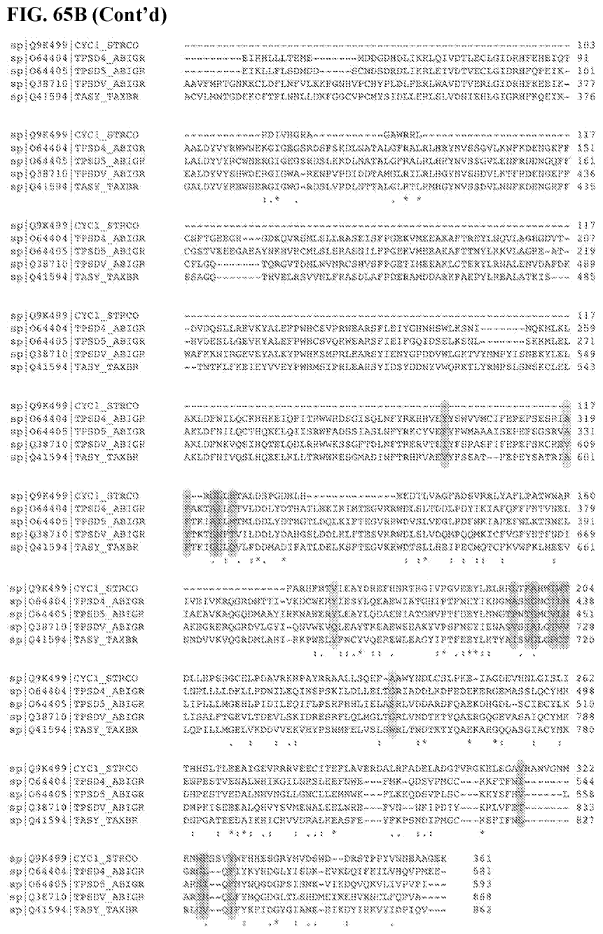

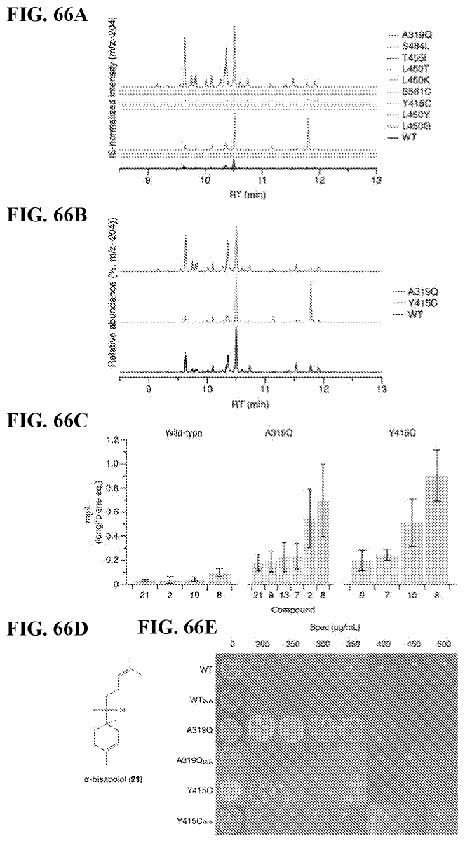

To search for evolutionarily accessible changes in the activity of GHS that might improve its ability to generate inhibitors of PTP1B, site-saturation mutagenesis was carried out at sites likely to influence the volume and/or hydration structure of the active site. The amino acids that line the active sites of terpene synthases may not be amenable to mutagenesis nor likely to shift product profiles. At notable extremes, mutations at catalytic residues (e.g., the DXDD motif) can inactivate the enzymes, while mutations at other sites can disrupt folding. Mutable, yet influential sites were searched by targeting poorly conserved residues that are likely to affect the volume or hydration structure of the active site. These features help dictate the conformation space, entropic constraints, and termination steps available to reacting intermediates. The following procedure was used to identify the residues for the site-saturation mutagenesis: (i) X-ray crystal structures of abietadiene synthase (ABS) from Abies grandis and taxadiene synthase (TXS) from Taxus brevifolia (GHS does not have a crystal structure) were aligned. (ii) All residues within 8 Å of the substrate analog (2-fluoro-geranylgeranyl diphosphate) of the class I active site of TXS were selected, and a subset of sites that differ between ABS and TXS were identified. (iii) The sequences of ABS, TXS, GHS, EIS, and 8-selenine synthase (DSS) from Abies grandis were aligned (FIG. 65). ABS and TXS were chosen as starting points because they are structurally similar enzymes with crystal structures; DSS, and EIS were chosen because they exhibit mutation-responsive product profiles. (iv) the following equation (Eq. 3-1) was used to score each site from step ii by its variability in volume and hydrophilicity across the five enzymes:

$$S = \frac{{σ}_{V}^{2}}{{n}_{v}} + \frac{{σ}_{HW}^{2}}{{n}_{HW}} \qquad Eq . V - 1$$

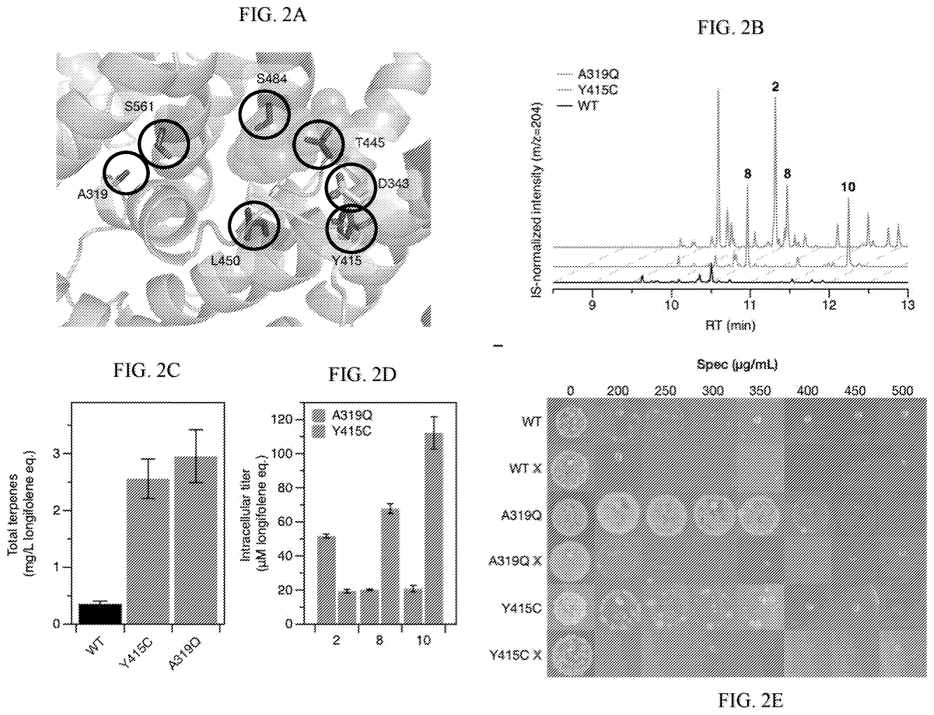

In this equation, σ_{V}² is the variance in volume, σ_{HW}² is the variance in Hopp-Woods index, and nᵥ and n_{HW} are normalization factors (e.g., the highest variances measured in this study). (v) Each site was ranked according to S and selected the six highest-scoring sites (FIGS. 2A-2E, FIGS. 65A-65E, FIG. 75). Two of the six sites identified with this approach (S484 and T445) had a strong influence on product profile of GHS in a previous analysis of single-site mutants; four shifted the products of EIS in separate analyses (Table 12). Consistency between the sites identified with the scoring function, S, provides a reasonable means of assessing the contribution of different residues to enzyme plasticity (e.g., finding residues that influence the product profiles of terpene synthases).

FIG. 2A shows a homology model for GHS shows residues targeted for site saturation mutagenesis (SSM). A substrate analogue (spheres) is positioned by aligning the crystal structure of 5-epi-aristolochene synthase (pdb entry 5eat). FIG. 2B shows Sesquiterpene production by GHS, GHS_{A319Q}, and GHS_{415C}. Chromatograms show the molecular ion (m/z=204), scaled to injection size as measured by the peak area of an internal standard (20 μg/mL methyl abietate, m/z=316). FIG. 2C shows Total terpene titers (mg/L, longifolene equivalents) for each strain. FIG. 2D shows Intracellular terpene titers of compounds 2, 8, and 10 (μM, longiolene equivalents). FIG. 2E shows The spectinomycin resistance conferred by mutants of GHS. Images show the growth of E. coli harboring pMBIS, pTS, and pB2H on agar plates seeded from drops of liquid culture. Mutations A319Q and Y415C enhance antibiotic resistance; X indicates inactive B2H (e.g., a substrate domain with a Y/F mutation). Error bars in B-D denote standard deviation for n≥3 biological replicates.

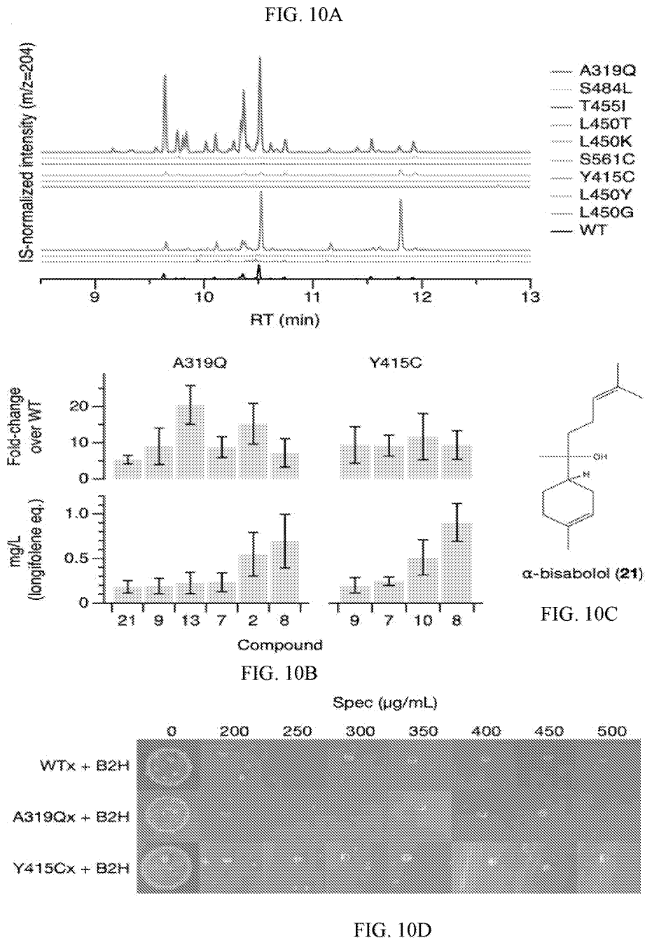

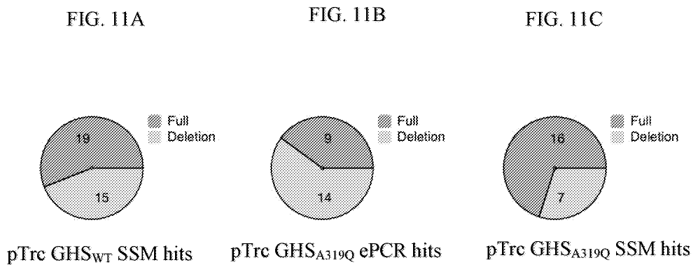

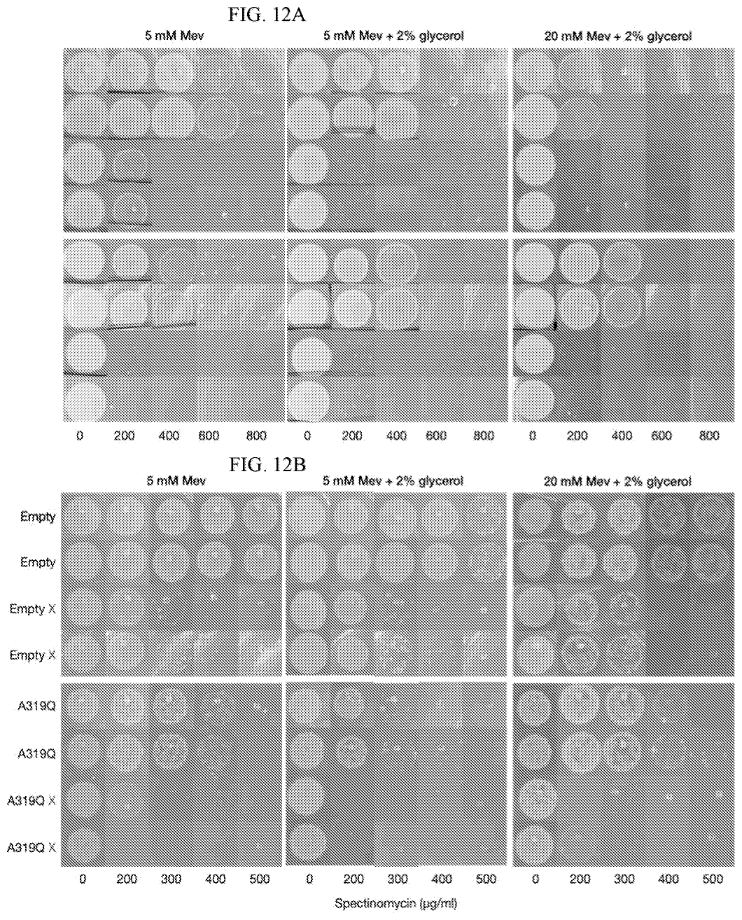

The B2H system was used to search for single-site mutants that confer a survival advantage. Briefly, the mutant library was transformed into cells harboring both the B2H system (pB2H) and the mevalonate-dependent pathway for FPP and IPP (pMBIS), picked colonies that grew at high concentrations of spectinomycin (e.g., 400-600 μg/ml), cloned identified mutations into a new plasmid (to reduce the effects of random mutations outside of the terpene synthase), and used GC-MS to examine the product profiles of the selected resistance-enhancing mutants. In the initial screen, two mutants were identified that generated major shifts in terpenoid production (FIGS. 2A-2E and FIGS. 10A-10D): A319Q has a similar product profile to the parent enzyme but achieved an eight-fold higher titer; Y415C has a more focused product profile (β-himachalene and himachalol are its major products) and affords a 7-fold higher titer (FIGS. 2A-2E) Intracellularly, A319Q produces β-himachalene and γ-humulene at concentrations sufficient to inhibit PTP1B at modest IC₅₀s (20 and 55 μM) and Y415C produces β-himachalene and himachalol at even higher concentrations (60 and 120 μM; FIG. 10); for comparison, previously identified sesquiterpene inhibitors with IC₅₀s ranging from 19-165 μM that improved spectinomycin resistance in the B2H (FIGS. 2A-2E).

FIGS. 10A-10D shows the product profiles of mutants identified in the screen of a single-site library (E. coli s1030+pTS+pMBIS+pB2H in 10-ml TB media). The chromatograms show extracted ions (m/z=204) scaled to injection size, measured by peak area of an internal standard (20 μg/mL methyl abietate, m/z=316).

A drop-based assay was used to examine the survival advantage conferred by both mutants (FIGS. 2A-2E). Each mutant enabled growth at higher concentrations of antibiotic than the wild-type enzyme; of the two, A319Q conferred the greatest survival advantage by improving antibiotic resistance significantly. Y415C yielded a modest improvement. Importantly, for both mutants, maximal resistance was associated with both an active GHS and an active B2H system (FIGS. 2A-2E, FIGS. 10A-10D); this suggests that cell survival under selection benefits from activate terpene synthases that generate PTP1B inhibitors (e.g., activators of the B2H system). Maximal resistance associating with the active B2H system indicates that PTP1B inhibition enhances resistance. Maximal resistance associating with the active terpene synthase indicates that GHS activity can enable PTP1B inhibition. In the presence of an inactive B2H system, the A319Q mutation also showed improved resistance compared to the WT or Y415C enzymes, indicating that this mutation improves growth independent of PTP1B inhibition; this could arise from improved tolerance for GHS expression in the host (e.g., improved solubility or optimized codon usage). This survival benefit was small compared to A319Q with an active B2H system, though, and combining A319Q with an inactivating D343A mutation led to no observed spectinomycin resistance ((FIGS. 10A-10D). Taken together, these findings suggest that A319Q and Y415C produce terpenoids that modulate the B2H (presumably, through PTP1B inhibition).

FIGS. 11A-11C shows defining gene deletions as (i) all or part of the terpene synthase missing in a Sanger sequencing result or (ii) no band, a band of incorrect size, or multiple bands in a colony PCR, the frequency of incomplete genes was quantified in (A) the site saturation mutagenesis screen using GHS WT as a template (FIG. 11A), (B) the error-prone PCR screen using GHS A319Q as a template (FIG. 11B), and (C) the site saturation mutagenesis screen using A319Q as a template (FIG. 11C). Labels in all charts indicate counts of full or incomplete gene.

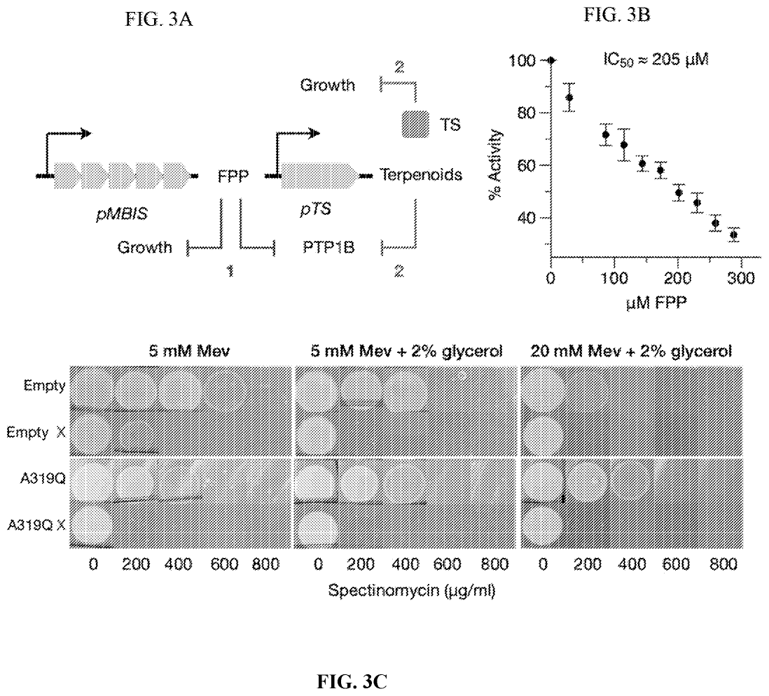

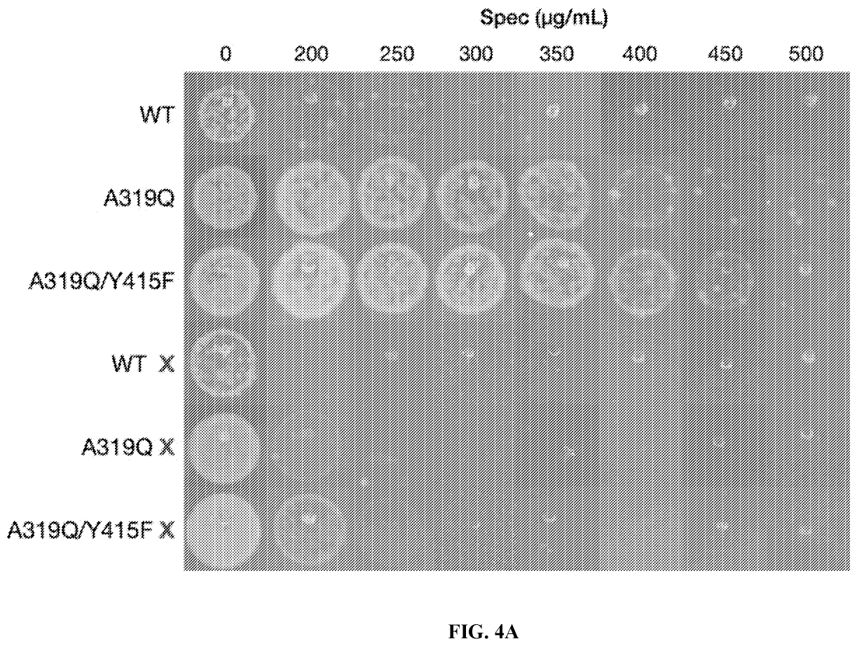

In the initial screen, many hits (e.g., cells that survived at high concentrations of spectinomycin) had an incomplete or missing GHS gene (44%, FIGS. 11A-11C). It was speculated that terpene synthase plasmids (pTS) lacking a full gene-potential cloning artifacts-might confer a survival advantage by promoting the accumulation of FPP. Low concentrations of this phosphate-containing hydrocarbon might activate the B2H system by inhibiting PTP1B, which binds to phosphorylated peptides, even though high concentrations can be toxic to E. coli (FIG. 3). Indeed, the in vitro kinetic data indicate that FPP can inhibit PTP1B with an IC50 of ˜200 μM (FIG. 3B), which is lower than previously reported intracellular concentrations for E. coli strains harboring the FPP-producing plasmid, pMBIS.

To bias the screen against B2H activation by FPP, which does not require terpene synthase activity, media conditions were searched that would disfavor this mode of survival by increasing FPP concentrations to toxic levels. In brief, concentrations of glycerol and mevalonate were increased—modifications known to enhance terpenoid production in E. coli—and evaluated the influence of these media conditions on antibiotic resistance. These conditions reduced the resistance conferred by incomplete TS plasmids (here, plasmids that lacked a GHS gene) but not the resistance afforded by A319Q (FIGS. 3A-3C and FIGS. 12A-12B). This finding suggests that media conditions (and associated changes in metabolic flux) can tune the solution space explored in our high-throughput screens.

FIGS. 3A-3B show the terpenoid pathway produces two potential inhibitors of PTP1B: FPP and terpenoids. Both routes to B2H activation have potential disadvantages: FPP is toxic to E. coli at high concentrations, and terpene synthases can misfold and inhibit growth. Initial rates of PTP1B-catalyzed hydrolysis of pNPP in the presence of increasing concentrations of FPP ([PTP1B]=50 nM; [pNPP]=5 mM). A linear fit provides a rough estimate of IC50 (inset). (C) The spectinomycin resistance conferred by an empty vector (e.g., pTS without a TS gene) and A319Q in different media. Media compositions that increase intracellular FPP concentrations (left to right) reduce the fitness advantage of the empty vector but leave the resistance conferred by GHSA319Q unaltered (X=a B2H system with a Y/F mutation in the peptide substrate). Images show the growth of E. coli strains harboring pMBIS, pTS, and pB2H on agar plates seeded from drops of liquid culture. Compositions previously shown to increase intracellular FPP concentration (left to right) reduce the fitness advantage of the empty vector but not that of GHSA319Q. Note: In the presence of 5 mM mevalonate and an inactive B2H system, the empty vector confers a greater survival advantage than the terpene synthase, suggesting that the terpene synthase can cause some cellular stress. Error bars in B denote standard error for n=3 independent measurements.

FIG. 12 shows replicates of a drop-plating experiment comparing empty vector to A319Q survival. In both cases, the empty vector shows reduced survival as glycerol and mevalonate concentrations increase; A319Q does not show this trend. The window of survival shifts based on OD₆₀₀ at the time of plating (A: OD₆₀₀ of all strains at time of plating ˜5.0, B: OD₆₀₀ of all strains at time of plating ˜2.0)

Bias of the screen against FPP accumulation was sought by creating a penalty for this mechanism of B2H activation. In brief, the effects of higher glycerol and mevalonate concentrations were tested in solid media on empty vector survival. In studies using similar terpenoid pathways (e.g., pMBIS and a terpene synthase expressed from the pTrc99 plasmid), these modifications have increased terpenoid production, suggesting they may cause higher flux through the MBIS pathway. As expected, these media changes reduced the antibiotic resistance conferred by the empty vector but left the resistance conferred by A319Q unchanged (FIG. 3 and FIG. 12). This result illustrates how changes in metabolic flux can rebalance different biological activities of small molecules in growth-coupled assays and alter the distribution of solutions to genetically encoded objectives.

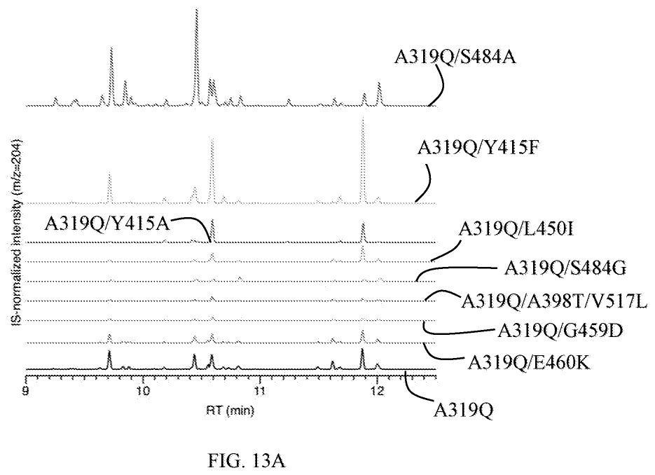

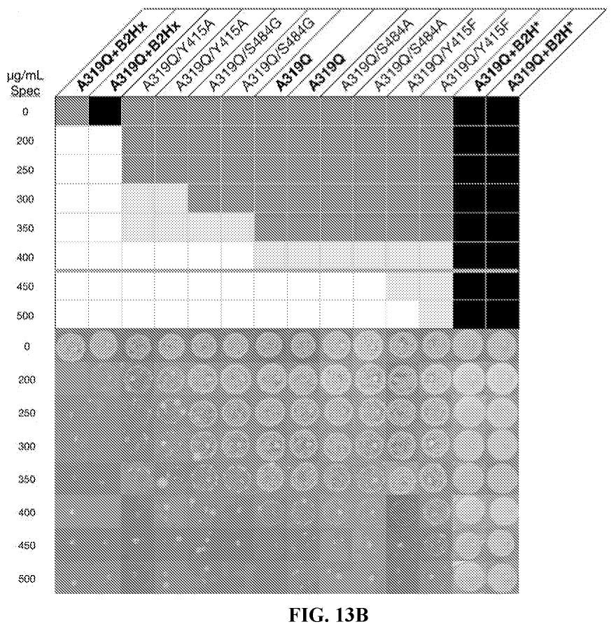

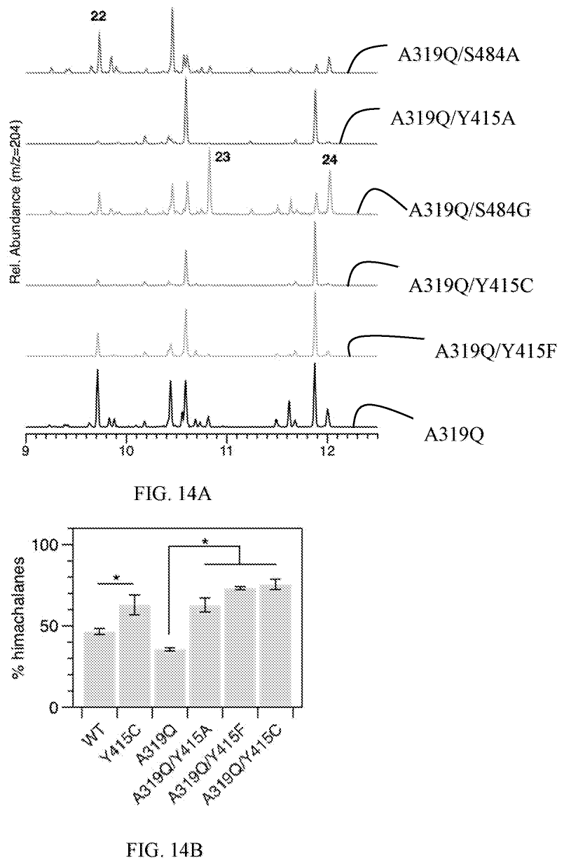

FIG. 13 shows the product profiles of mutants found in site-saturation mutagenesis and error-prone PCR screens using GHSA319Q as the parent template (E. coli s1030+pTS+pMBIS+pB2H in 10-ml TB media). The chromatograms show extracted ions (m/z=204) scaled to injection size, measured by peak area of an internal standard (20 μg/mL methyl abietate, m/z=50-500). A319Q/Y415C was not found in the screen, rather, it was manually constructed to combine the two best mutations from the single-site library

To evolve the terpenoid pathway further, the additional rounds of mutagenesis were carried out on GHS. Mutations were searched for that might improve upon A319Q by using SSM and error-prone PCR (ePCR). For SSM, we selected the five remaining sites identified with Eq. 3-1; for ePCR, a homology model was generated all residues within 8 Å of the substrate analog used to select SSM sites were targeted (FIG. 2A). The resulting libraries were screened as before using solid media conditions expected to bias against FPP accumulation. Hits identified under the new media conditions exhibited a lower frequency of incomplete GHS genes for the SSM library but not the ePCR library (FIG. 11B and FIG. 11C). Rates of incomplete or missing GHS genes was observed from the error-prone PCR screen that were higher than in the initial screen (61%, FIG. 11), while the site saturation mutagenesis screen showed reduced frequency of this phenomenon (30%, FIG. 11); this discrepancy could be related the different homology regions used for cloning or different proportions of variants that outcompete the empty vector in each library. Media that increases FPP accumulation thus appears to reduce the incidence of incomplete pTS plasmids but does not eliminate them. These hits may be more common in ePCR libraries, where non-functional genes are typically more abundant, though differences in library preparation (e.g., homology regions used for cloning) cannot be ruled out as a possible cause. The eight different mutations found in colonies growing on high concentrations of spectinomycin (500-800 μg/ml) re-cloned and analyzed: four of these were error-prone PCR hits showing no major changes in product profile compared to A319Q (in fact, two of them appear to reduce terpenoid production), and the remaining four were saturation mutagenesis hits, including three with notable changes in terpenoid profiles—A319Q/Y415F, A319Q/S484G, and A319Q/S484A, (FIGS. 13A-13B).

The eight hits that grew at high concentrations of spectinomycin (400-600 μg/ml) were examined: five from SSM and three from ePCR (FIG. 13A). Two SSM mutants and all three ePCR mutants left product profiles unchanged and/or reduced terpenoid titer (relative to A319Q). The three remaining SSM mutants exhibited major shifts in terpenoid production: A319Q/Y415F, A319Q/S484G, and A319Q/S484A. Drop-based plating was used to confirm their survival advantage (FIG. 13B). Among the mutants experimented with, A319Q/Y415F enhanced antibiotic resistance (relative to A319Q). Significant enhancement of antibiotic resistance was not detected with the other mutants experimented with (e.g., A319Q/S484A). One mutant reduced antibiotic resistance slightly (A319Q/S484G). A319Q/S484G produced new terpenoids (FIG. 13A). This mutant highlights the potential for neutral or mildly deleterious evolutionary steps to access new activities, which can serve as starting points for alternative routes to improved fitness.

Certain features of mutated synthases enhance antibiotic resistance, while others do not. For example, some mutated terpene synthases enhance antibiotic resistance because they produce inhibitors that activate the B2H (inhibitors of PTP1B in our paper), while in general, mutants that do not make an inhibitor do not enhance resistance as much as those that do. Whether a mutated synthase enhances antibiotic resistance is also impacted by other sources of toxicity. For example, mutants might form toxic aggregates, produce bactericidal metabolites or metabolites that disrupt cellular function, or that compete more effectively for essential metabolic intermediates than other enzymes in the cell (a sort of siphoning off effect). The resistance as described herein is likely a non-linear combination of multiple biochemical properties, which are challenging to predict from structure or sequence data alone. Properties that have been examined include inhibitor production (the goal of our genetically encoded system is to find mutants that produce inhibitors), enzyme toxicity (some mutations also make the GHS enzyme less toxic, perhaps, for one of the reasons shown above), and enzyme solubility (some mutations appear to improve stability).

FIGS. 4A-4D show a drop-based plating of sequential mutations that improve fitness. “X” indicates an inactive B2H. Total terpenoid titers and product distribution of variants in; only the two most abundant products in each strain are shown in the pie charts in FIG. 4C for simplicity. The fold-change in titers of the indicated products for A319Q/Y415F compared to the enzymes indicated by “X”. Mutants tested were all found through evolution except A319Q/Y415C, which was a rational combination of mutations from the single-site screen. Top: himachalol, bottom: β-himachalene. Dashed lines indicate a fold-change of 1. Error bars in FIG. 4B denote propagated standard deviation for n≥3 biological replicates. Mutations to the Y415 residue shift production towards himachalane-type sesquiterpenes. Hypothesized mechanism for conversion of FPP to humulane or himachalane-type sesquiterpenes.

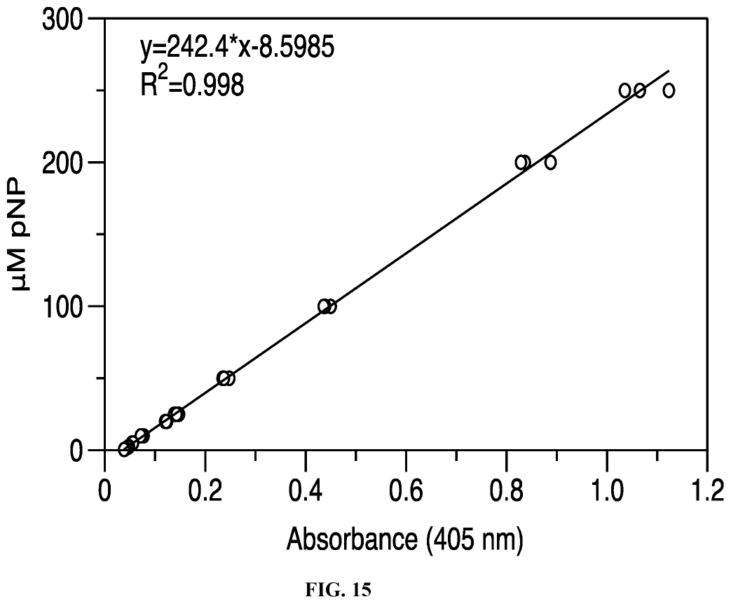

FIG. 15 shows a standard curve shows the absorbance (405 nm) of difference concentrations of p-nitrophenol (pNP). This standard curve links the concentration of pNP to the absorbance of this molecule (405 nm) in 100 μL of buffer (50 mM HEPES, pH=7.3) in a 96-well plate.

FIGS. 14A-14B show extracted ion chromatograms (m/z=204) normalized such that the largest peak height=1. Unidentified compounds are labeled (22-24). Compounds 23 and 24 are unique to A319Q/S484G. Total terpenes produced by interesting single-site and multi-site mutants.

The drop-based plating of these mutants was performed, as before. A319Q/Y415F conferred a survival advantage and exceeded the growth of A319Q (FIGS. 4A-4D). This mutant's product profile resembles Y415C (A319Q/Y415F's major products are also β-himachalene and himachalol) but improves total terpene titer 1.7× over A319Q and 2× over Y415C (FIG. 4 and FIG. 14). Interestingly, in the presence of an inactive B2H, A319Q/Y415F conferred more resistance than both the WT enzyme and A319Q, suggesting it provides a B2H-independent growth benefit beyond that of A319Q (although, again, this benefit was small compared to the mutant paired with an active B2H, FIG. 4). A319Q/S484A was a fitness neutral mutant despite improving total terpene production in a 4 mL culture, including a large increase in γ-humulene and an unidentified compound (22, FIGS. 13A-13B and FIGS. 14A-14B). A319Q/S484G conferred slightly less resistance to spectinomycin than A319Q (this hit was picked from a plate with the least stringent selection condition tested: 500 μg/ml) despite a 1.5× increase in total terpenoids, including two unidentified compounds unique to this strain (23 & 24, FIGS. 13A-13B, FIG. 15, and FIGS. 14A-14B). Importantly, the major differences between the profiles of the S484 mutants and A319Q/Y415F were in the production of β-himachalene and himachalol; these compounds were much more abundant in A319Q/Y415F. Compared to other, less spectinomycin-resistant, mutants the procedure found through evolution, A319Q/Y415F consistently produced greater amounts of himachalol; titers of β-himachalene, however, were comparable between A319Q/Y415F, A319Q, and Y415C (FIG. 4). Taken together, these results suggest (i) A319Q/Y415F confers a survival advantage above both the WT and A319Q enzymes through B2H modulation, and (ii) himachalol and, to a lesser extent, β-himachalene are likely the molecules responsible for this survival advantage and merit further investigation.

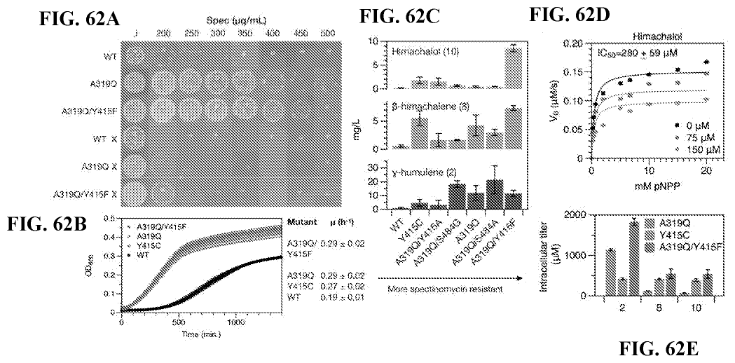

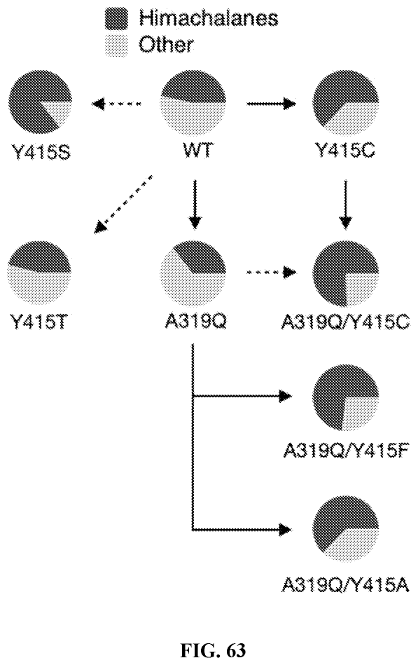

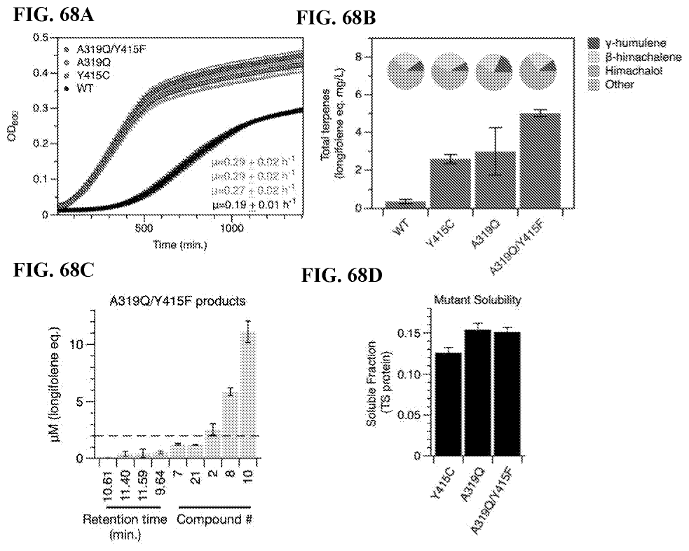

Mutants A319Q and A319Q/Y415F enhanced antibiotic resistance (albeit, mildly) in the presence of an inactive B2H system (FIG. 4A). Significant enhancement of antibiotic resistance was not detected with Y415C. To evaluate the influence of these mutations on enzyme toxicity, E. coli was transformed with plasmids harboring the wild-type, Y415C, A319Q, and A319Q/Y415F variants of GHS and grew the transformed strains in liquid culture. These strains did not contain the FPP pathway or the B2H system, so neither FPP toxicity nor PTP1B inhibition affected growth. Interestingly, all three mutants reduced the lag phase and improved the specific growth rate of transformed strains (relative to GHS; see FIG. 62B); this effect suggests that all three protein mutants exhibited lower toxicity than GHS. In addition to A319Q/Y415F's enhanced production of the inhibitor, himachalol, both A319Q and A319Q/Y415F show improved solubility compared to Y415C (as shown with respect to FIG. 68D). Broadly, analysis of different mutants indicates that a reduction in enzyme toxicity in E. coli is a measurable property of GHS mutants that allows some mutants to enhance antibiotic resistance; they also illustrate how growth-coupled screens can improve the compatibility of nonnative enzymes with heterologous expression hosts.

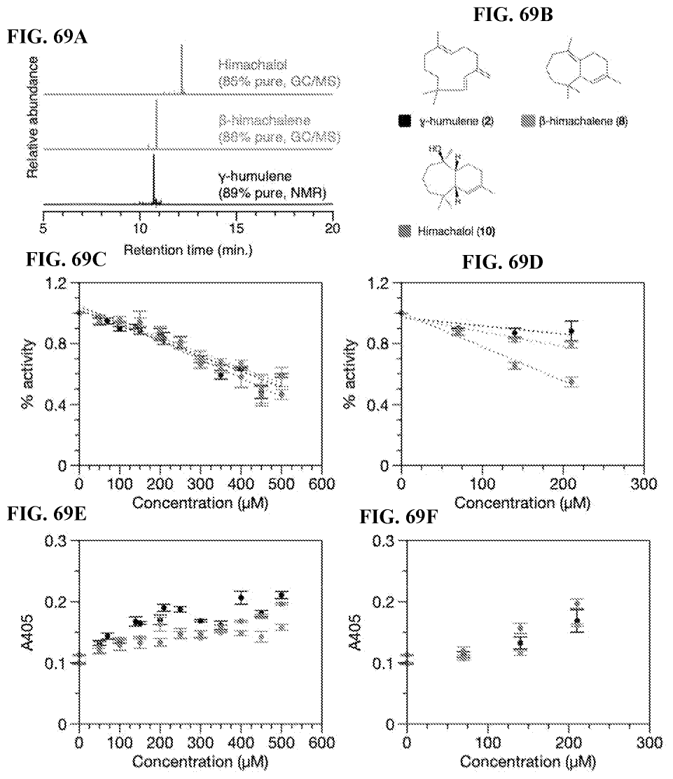

Mutant A319Q/Y415F afforded the most spectinomycin resistance of any variant (FIG. 13B) and used an active B2H for maximal resistance (FIG. 4A)—an indication that it generates an inhibitor of PTP1B. Inhibitory terpenoids were searched by comparing the product profiles and fitness advantages of various mutants, starting with A319Q/Y415F and A319Q. The double mutant has three major products: γ-humulene, β-himachalene, and himachalol. To assess the inhibitory effects of these molecules, they were purified (>85% purity) from cell cultures (γ-humulene and himachalol) or a commercial preparation (β-himachalene) and their influence on PTP1B activity was examined (e.g., their ability to inhibit PTP1B-catalyzed hydrolysis of p-nitrophenyl phosphate were measured). Intriguingly, himachalol, which A319Q/Y415F generates at a uniquely high titer, had an IC₅₀ that was about two-fold lower than its intracellular concentration (e.g., an IC50 of 280±59 μM and a titer of 543±106 μM; FIGS. 62C-62E and FIGS. 68B-C). By contrast, γ-humulene and β-himachalene, which A319Q/Y415F generates at similar titers to mutants that confer less resistance, were less inhibitory (though, their low solubilities precluded IC₅₀ estimates; FIG. 69). Our analysis of himachalol suggests that enhanced inhibitor production is a measurable property of GHS mutants that allows some mutants to enhance the antibiotic resistance of B2H-encoded E. coli cells.

Y415C also generates large amounts of himachalol (e.g., intracellular concentrations of 389±33 μM) and merits further discussion. Like A319Q/Y415F, Y415C improved the specific growth rate of E. coli in liquid culture; however, unlike the double mutant, it failed to improve antibiotic resistance when paired with an inactive B2H system in our selection assay. At first glance, these results seem contradictory, but they probably reflect the different cellular stresses imposed by the two experiments. To collect growth curves, E. coli strains were used that lack both the FPP pathway and the B2H system; for selection experiments, both were included. As a result, the selection experiments place four additional stresses on the cell: (i) the isoprenoid pathway, which generates FPP, a toxic intermediate, (ii) the B2H system, which has no apparent toxicity but requires cellular resources for plasmid maintenance and constitutive protein expression, (iii) the antibiotics required to maintain pMBIS and pB2H, and (iv) spectinomycin (the variable selection pressure used in our assay). It is speculated that these stresses may accentuate differences in the toxicity of GHS mutants. This theory was explored, in part, by comparing the soluble fractions of Y415C, A319Q, and A319Q/Y415F overexpressed in E. coli (FIG. 68D). Indeed, A319Q and A319Q/Y415F had a 20% higher soluble fraction than Y415C, a finding consistent with their potential to exhibit reduced toxicity under some growth conditions. Our analysis of the solubility of different mutants suggests that enhanced enzyme solubility (or stability) is a measurable property of GHS mutants that allows some mutants, but not others.

Comparisons of GHS mutants, taken together, indicate that the pronounced fitness advantage of A319Q/Y415F results from both (i) its ability to overproduce himachalol, a PTP1B inhibitor, and (ii) its reduced cellular toxicity. Importantly, himachalol titer of A319Q/Y415F is over fifty-fold higher than that of the wild-type GHS; this mutant illustrates the efficiency with which terpene synthases can adapt to produce new biologically active molecules.

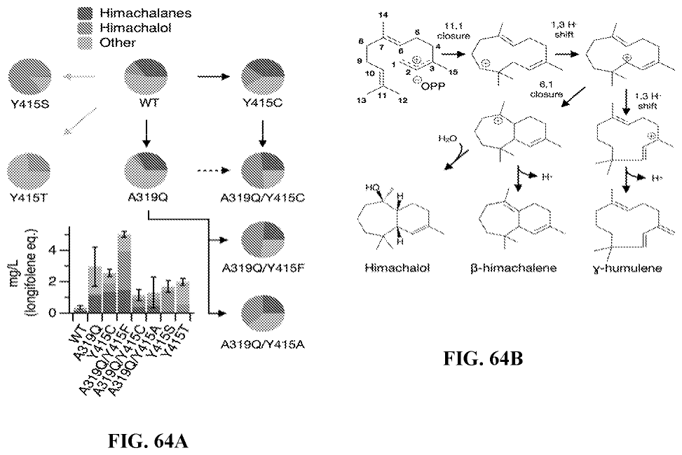

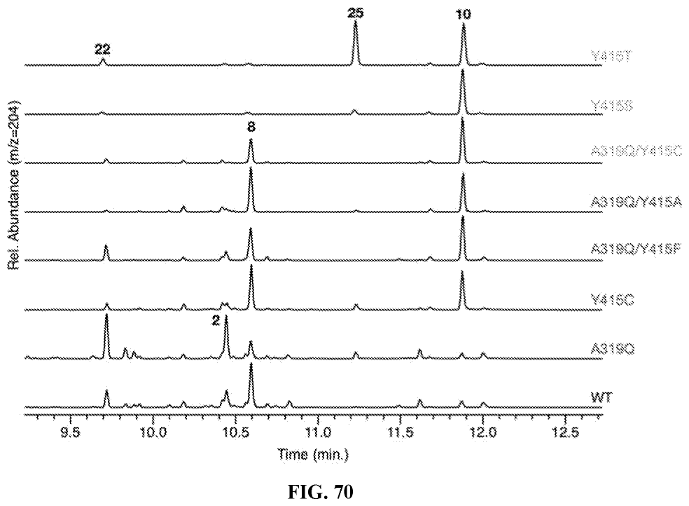

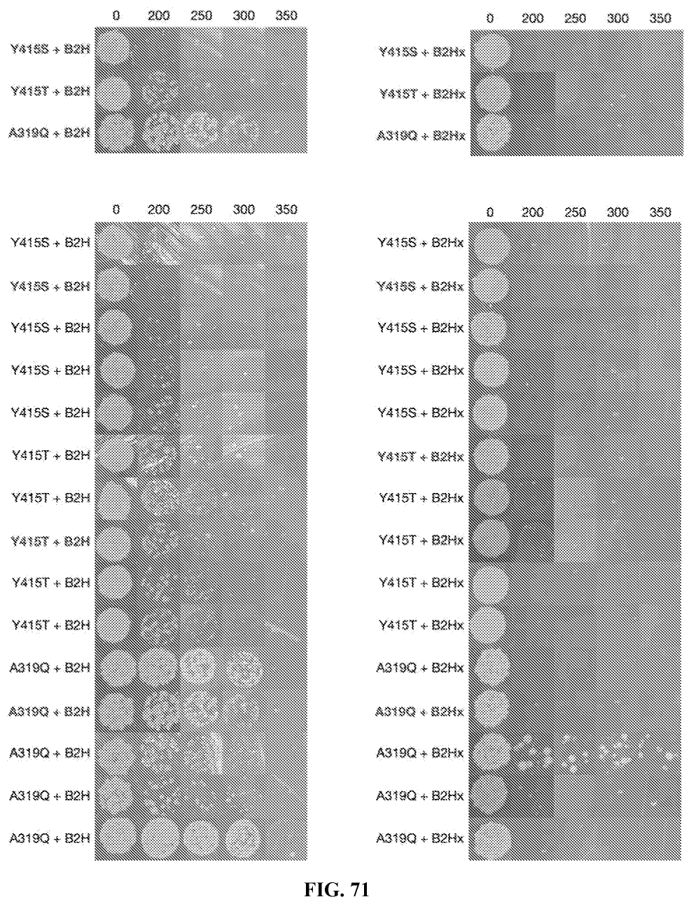

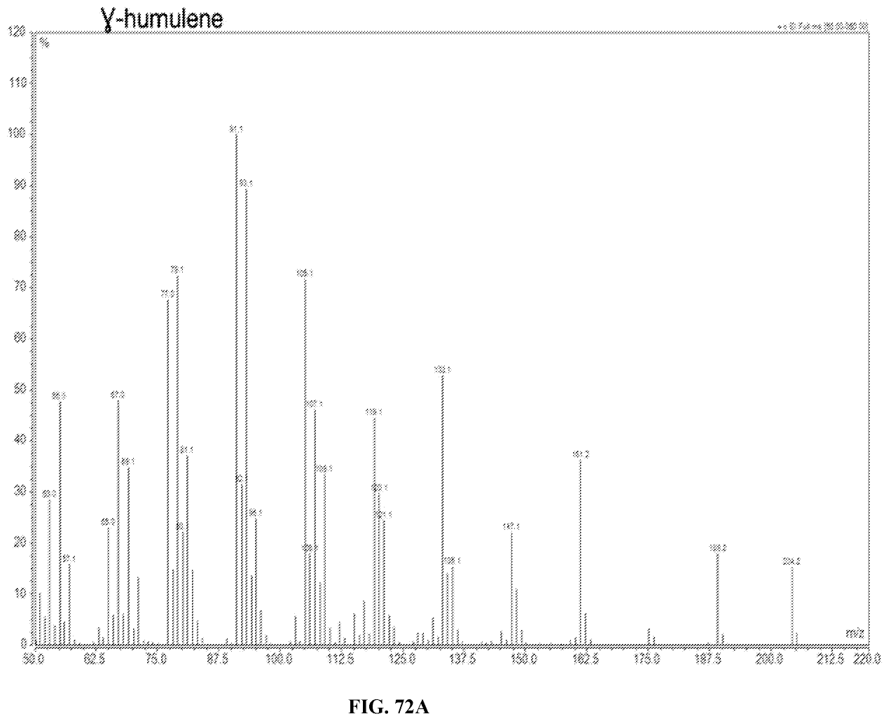

A single carbocation intermediate can undergo either (i) a 6,1-ring closure to form himachalanes or (ii) a 1,3-hydride shift to form humulanes (FIG. 64B) in GHS. In a screen of GHS libraries, three mutations were identified at Y415 that shift the product profile toward himachalane-type sesquiterpenoids (7-10): Y415A, Y415C, and Y415F (FIG. 64A and FIG. 14B). The mutant residues exhibit different sizes and chemical functionalities, but all lack a hydroxyl group. It is speculated that the removal of this group might promote 6,1-ring closure and immediate deprotonation or quenching by a water molecule; rapid quenching is consistent with the enhanced himachalol titers generated by Y415 mutants (FIG. 64A and FIG. 70). The hypothesis was tested by examining the product profiles of Y415S and Y415T, which were prepared with site-directed mutagenesis. Interestingly, Y415S produced mainly himachalol, while Y415T generated both himachalol and an unidentified product (FIG. 64A). Both mutants produced less himachalol than A319Q/Y415F. Both mutants did not significantly improve antibiotic resistance compared to A319Q when paired with active and inactive B2H systems (FIG. 71); this may be due to solubility issues similar to those exhibited by Y415C, which can help explain the lack of these mutants emerging as a dominant population in our selection assay. Overall, the enhanced himachalol production afforded by chemically varied mutations at Y415 suggest that this residue occludes or constrains (via hydrogen-bonding) a water molecule that, with the additional volume or freedom afforded by smaller side chains, quenches the carbocation precursor of himachalol. Previous studies of class II terpene synthases have observed a similar effect upon mutating a conserved histidine to alanine. Additional biophysical analyses (e.g., X-ray crystal structures and molecular simulations) may be used to elucidate the detailed mechanism by which Y415 restricts himachalol production, as a single mutation can affect the size, contour, flexibility, and hydration of the active site. Nonetheless, the Y415 mutants illustrate the early insights afforded by high-throughput screens that can find multiple mutants with similar product profiles.

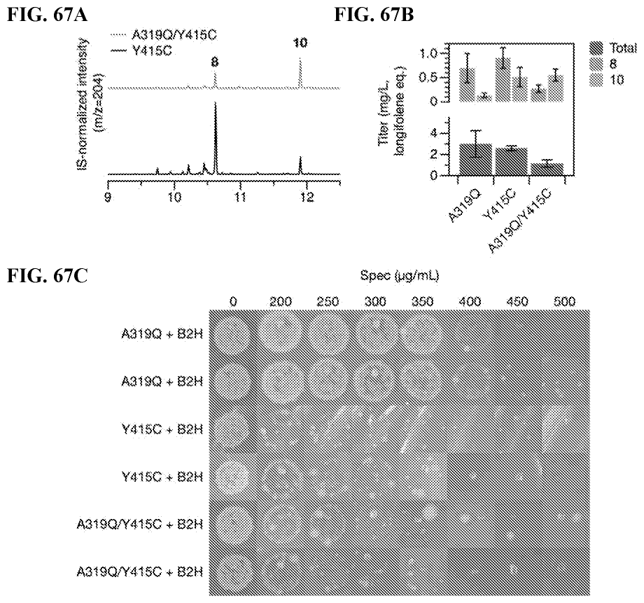

Rational combination of the two best-performing mutants from the single-site screen was investigated. Unlike previous studies, combining mutations did not yield additive effects: compared to Y415C, the A319Q/Y415C mutant showed similar survival characteristics and an ˜57% reduction in terpenoid titers (the profiles, though, were shifted to β-himachalene and himachalol in both strains, FIGS. 10A-10D, and FIGS. 14A-14B) and antibiotic resistance was left unchanged (FIGS. 67A-67C); compared to A319Q, the mutant showed less spectinomycin resistance along with a decrease in titer. Similar survival phenotypes between the double mutant and Y415C, despite a decrease in titer, suggests the addition of A319Q may simultaneously impact catalysis negatively and enzyme tolerance positively, but further analysis is required to fully characterize the effects of each mutation alone and together. These results do not necessarily disagree with previous reports of mutations being additive in GHS: synergistic effects have only been reported for mutations that are either (i) near the active site and shift the product profile towards the same terpenes or (ii) far from the active site and enhance enzyme tolerance. Because A319Q and Y415C are both in the active site and affect the product profile in distinct ways, it is perhaps less surprising that their combination does not lead to improvements in production or growth in the system.

Throughout the evolution effort, three different mutations were identified at residue Y415: alanine, cystine, and phenylalanine. Curiously, each mutation at this position shifted the product profile of the enzyme towards himachalane-type sesquiterpenoids (products 7-10, FIGS. 4A-4D). The mutated residues all lack the hydroxyl group present in the native tyrosine residue; they otherwise span a wide range of sizes and functional groups. Prior work suggests a single carbocation intermediate undergoes either a 1,6-ring closure to form himachalanes or a 1,9-hydride shift to form humulanes (FIGS. 4A-4D). The findings suggest a wide range of substitutions at Y415 favor 1,6-ring closure; thus, the hydroxyl group of the tyrosine may be important for producing sesquiterpenes that proceed through the 1,9-hydride shifted cation.

This study evolved γ-humulene synthase to solve a genetically encoded problem in E. coli: inhibition of a medicinally relevant enzyme. This is the first use of a growth-coupled selection to guide terpene synthase evolution towards production of a biologically active molecule. Potential inhibitors of PTP1B were identified that merit further investigation: himachalol and β-himachalene. Importantly, the final mutant showed a 50-fold increase in himachalol production over the wild-type enzyme. This remarkable improvement in titer of a minor product through only two rounds of evolution reveals a powerful feature of the approach for molecular discovery: mutations that enhance the production of compounds that activate the two-hybrid system will be enriched, making their isolation for characterization easier (isolating these compounds from wild-type γ-humulene synthase, which produces much lower titers, would be much more difficult). Many other terpene synthases, as well as functionalizing enzymes like cytochrome P450s, produce different products with various titers in response to mutations, suggesting the approach could be used to evaluate large and diverse sets of molecules by evolving a minimal number of starting pathways to solve genetically encoded problems.

There were challenges to evolving a metabolic pathway under a selection pressure that were identified. Farnesyl pyrophosphate, an intermediate in sesquiterpene biosynthesis, was found to be an inhibitor of PTP1B; its production enriched variants in which the evolving terpene synthase gene (which, for GHS, is slightly toxic to E. coli) was removed from the cell. Strategies for reducing the viability of “uninteresting” solutions, like a promoter that responds to FPP accumulation by expressing a toxic gene, could further bias the cell towards producing a more interesting molecule. Intermediates may trigger selection systems in other natural product pathways as well; systematically characterizing their effects may be necessary to ensure the target(s) for directed evolution will be retained following selection.

Many natural terpenoid pathways evolved to improve the fitness of living systems in response to specific biochemical challenges (e.g., cellular responses to both biotic and abiotic stresses). In this study, a B2H system was used to define an artificial challenge—the inhibition of PTP1β—and evolved a terpene synthase to address it. The screen of a relatively small library of GHS mutants (e.g., SSM at 6 sites) identified single and double mutants that improved the fitness of B2H-encoded E. coli cells by reducing GHS toxicity and/or by increasing inhibitor production. These distinct biochemical traits highlight the multi-objective optimization problems that guide the evolution of specialized secondary metabolites in biological systems.

Terpene synthases have been the subject of a myriad of detailed enzymological studies, but they remain challenging to engineer. Mutations that alter their product profiles often reduce catalytic activity, and substitutions required to generate specific products are challenging to predict de novo. The growth-coupled assays disclosed herein identified a combination of mutations in GHS that improve the titer of a minority product—a terpene alcohol that inhibits PTP1β—by over fifty-fold, and enabled the isolation of a residue where mutations can improve water capture—a historically challenging feat, given the complexity of the carbocation cyclization cascade and the contributions of water. Sesquiterpene synthases that generate a single hydroxylated product are rare, but the analysis described herein allowed building one: Y415S, which produces mainly himachalol. The findings suggest that activity-guided screens—and, perhaps in the future, screens carried out with generalist biosensors for specific classes of terpenoids—can accelerate the discovery of active, functionally distinct variants of terpene synthases, which are valuable starting points for structure-function studies and protein engineering.

A genetically encoded objective has several important differences from some complex biochemical challenges encountered in nature (e.g., inter-organism communication). First, the target of inhibition is located within the same cell—and within the same cellular region, the cytosol—as the terpenoid pathway, so terpenoid transport between cells is not a selection criterion. Second, two system properties—an overabundance of terpenoid precursor and inefficient terpenoid export—lead to high intracellular concentrations that make potent inhibitors unnecessary. Notably, the analysis culminated in a double mutant with major products that were easy to purify; mutants with potent, low-abundant inhibitors may have been overlooked. New approaches to reduce intracellular titer (e.g., a reduction in precursor supply) or to survey minority products could yield more potent molecules. Finally, the E. coli cells used in this study lack P450 monooxgenases and other terpenoid-functionalizing enzymes that could generate more soluble or potent molecules. Future efforts to integrate these enzymes into terpenoid pathways could expand the solution space explored in activity-guided screens.

The study of evolution focused on a single enzyme in a terpenoid pathway, but in nature, enzymes do not evolve one at a time and the intermediate metabolites are not strictly acted upon by individual enzymes in a sequence. As an example, plant diterpenes are often produced through two cyclization steps (in distinct active sites) that can be carried out by multiple enzymes, and the resulting diterpenoids can then be acted upon by multiple cytochrome P450s (whose products can sometimes react even further with the same enzymes). Each of these enzymes can simultaneously undergo random mutation/recombination to yield pathways producing different final products. Extending the work in this study to multiple component pathways like those of plant diterpenoids (within the limits of heterologous expression in E. coli) could be a powerful approach for enlarging the chemical space being searched for inhibitors of PTP1B (or another genetically encoded objective). Multicomponent pathways could also be used with the two-hybrid system to explicitly investigate the propensity for a pathway to produce a biologically active compound when it is evolved sequentially (e.g., one enzyme at a time) versus when multiple components are evolved at once.

E. coli DH10B, chemically competent NEB Turbo, and electrocompetent One Shot Top10 (Invitrogen) cells were used for cloning and library preparation. E. coli BL2 (DE3) cells were used to express proteins for in vitro studies, and E. coli s1030 for all B2H analyses, and DH5α for terpenoid isolation. When necessary, the chemically competent and electrocompetent cells were generated with well documented protocols (RbCl and washing, respectively).

Farnesyl pyrophosphate (FPP) and methyl abietate from Santa Crua Biotechnology; tris(2-carboxyethyl) phosphine (TCEP), bovine serum albumin (BSA), M9 minimal salts, phenylmethylsulfonyl fluoride (PMSF), and DMSO (dimethyl sulfoxide) from Millipore Sigma; longifolene, glycerol, bacterial protein extraction reagent II (B-PERII), and lysozyme from VWR; cloning reagents from New England Biolabs; and α-bisabolol and all other reagents (e.g., antibiotics and media components) were purchased from Thermo Fisher. Cedarwood oil (for β-himachalene isolation) was purchased from King Soopers. The mevalonate was prepared by mixing 1 volume of 2 M DL-mevalanolactone with 1.05 volumes of 2 M KOH, followed by incubation at 37° C. for 30 minutes. Vanillin-sulfuric acid solution was prepared by adding 7 g of vanillin and 1.3 mL of concentrated H₂SO₄ to 200 mL of methanol. Gene sources for new plasmids are outlined in Table 13.

All plasmids were constructed by using Gibson Assembly. Table 2 describes the composition, antibiotic resistance, and availability of all final plasmids. Table 3 and Table 4 list the primers used for plasmid assembly. NEB Turbo was used for all cloning, BL21 (DE3) for all protein expression, DH10B for large-scale terpenoid production, and s1030 for all B2H experiments.

A homology model of GHS was constructed by using SWISS-MODEL with α-bisabolene synthase (pdb entry 3SAE) as a template. This software package uses ProMod3 to build models from a target-template alignment, which preserves the structures of conserved regions and remodels insertions and deletions with a fragment library.

Multiple sequence alignment were carried out for the amino acid sequences of ABS, TXS, GHS, EIS, and DSS by using Clustal Omega (FIGS. 65A-65B). This program uses seeded guide trees and HMM profile-profile techniques to generate alignments between three or more sequences.

Libraries of enzyme mutants were prepared by using site-saturation mutagenesis (SSM) and error-prone PCR (ePCR). For SSM, the procedure performed the following steps: (i) The genes were amplified with primers containing degenerate codons (NNK) at the residues of interest. (ii) The amplified genes were digested with DpnI, purified them with gel electrophoresis, and used circular polymerase extension cloning (CPEC) to integrate them into plasmids (e.g., pTS). (iii) Heat shock was used to transform the fully assembled plasmids (10 μL) into chemically competent NEB Turbo cells (100 μL). (iv) After 1 hour of shaking (37° C., 225 RPM) in 1 mL SOC, serial dilutions were used on LB agar plates (20 g/L agar, 10 g/L tryptone, 10 g/L sodium chloride, 5 g/L yeast extract, 50 μg/ml carbenicillin) to ensure library size was greater than 10-fold the maximum number of transformants required for full coverage of all possible codons (e.g., greater than 2,240 transformants for a single site saturation mutagenesis library using NNK codons), and all remaining cells were plated over several plates for overnight growth (37° C.). (v) 3-5 colonies were sequenced to verify the presence of mutated genes. If over 50% of the colonies were missing mutations, the library was remade. (vi) The plates were scraped into LB media (10 g/L tryptone, 10 g/L sodium chloride, 5 g/L yeast extract) and miniprepped the final transformants to recover the DNA Library (E.Z.N.A. Plasmid DNA Mini Kit, Omega). (vii) All final libraries were frozen in MilliQ water at −20° C.

For ePCR, the same procedure was carried out with the following modifications: (i) The structures of γ-humulene synthase (modeled) and 5-epi-aristocholene synthase (PDB entry 5EAT) were aligned (ii) The Genemorph II kit (Agilent) was used to amplify residues 304-593 (comprising all amino acids within 8 Å of the substrate analog from PDB structure 5EAT aligned to the homology model) with a high error rate (50 ng template DNA: predicted 9-16 mutations/kb), and the procedure dialyzed the final plasmid mixture into MilliQ water for 2 hours. (ii) Two 100-μL aliquots of electrocompetent One Shot Top10 cells were transformed with 10 μL of the dialyzed CPEC reaction, and each aliquot was recovered in 900 μl SOC for 1 hour. (iii) The outgrowths were pooled, plated serial dilutions on 100 mm petri dishes, and plated the remaining cells on a single large bioassay dish (245 mm×245 mm×25 mm with 20 g/L agar, 10 g/L tryptone, 10 g/L sodium chloride, 5 g/L yeast extract, and 100 μg/ml carbenicillin). The cells were grown at 30° C. overnight to minimize lawn formation, scraped the resulting colonies, and froze the final libraries as above.

The SSM libraries were screened in eight steps: (i) 100 ng of each frozen DNA library (one per site) was pooled and the pooled library was dialyzed into MilliQ water for two hours. (ii) 10 μL was electroporated of the dialyzed library into a 100-μL aliquot of E. coli s1030 cells harboring a mevalonate-dependent isoprenoid pathway producing IPP and FPP (pMBIS) and the two-hybrid system (pB2H), and the cells were recovered in 900 μL SOC for 1 hour (37° C., 225 RPM). (iii) The serial dilutions of each transformation reaction were plated on LB agar supplemented with antibiotics for plasmid maintenance (50 μg/ml kanamycin, 50 μg/ml carbenicillin, 10 μg/ml tetracycline, and 34 μg/ml chloramphenicol) to estimate screening coverage. (iv) The remaining transformants were grown in 50 mL Terrific Broth (TB: 12 g/L tryptone, 24 g/L yeast extract, 20 mL/L glycerol, 2.28 g/L KH2PO4, 12.53 g/L K2HPO4, plasmid antibiotics) overnight (37° C., 225 RPM). (v) An aliquot of each culture was diluted in 1:75 in 4.5 mL TB (pH=7.0 with plasmid antibiotics) and grew this dilution to an OD₆₀₀ of 0.3-0.6 (37° C. and 225 RPM). (vi) 500 μM IPTG and 20 mM mevalonate were added, and each induced culture grew for 20 hours (22° C. and 225 RPM). (vii) Each culture was diluted to an OD₆₀₀ of 0.001 and spread 100 μL on LB agar plates (pH=7.0) supplemented with 20 mM mevalonate, 500 μM iPTG, 20 mL/L glycerol (omitted in single-site library screens), plasmid antibiotics, and varying concentrations of spectinomycin. For steps ii-vii, a plasmid harboring the parent terpene synthase was included into each library as a control. (viii) The cells were grown at 22° C., checking for colony growth every 24 hours. The hits were picked from plates for which the library produced a greater number of colonies than the control (e.g., the parent template used for mutagenesis). The ePCR libraries were screened in an analogous fashion (steps ii-viii).

To identify hits meriting further analysis, the terpene synthase gene from either a plasmid extraction or PCR amplifications were sequenced. The mutations identified by this process were introduced into a new pTrc vector harboring GHS (to minimize the impact of random mutations occurring outside of the targeted gene). The re-cloned mutants were transformed into s1030 cells harboring pB2H and pMBIS and plated on LB agar supplemented with antibiotics for plasmid maintenance (50 μg/ml kanamycin, 50 μg/ml carbenicillin, 10 μg/ml tetracycline, and 34 μg/ml chloramphenicol). Colonies were picked to determine product profiles in 4 mL cultures (see below); mutants producing different or greater amounts of products were subjected to drop-based plating to measure spectinomycin resistance.

For the SSM libraries, it was aimed to screen library sizes of at least ten times the maximum number of variants. The first SSM library was constructed by pooling six single-site libraries in an equimolar ratio; it had a maximum diversity of 120, and 15,000 and 9,000 mutants were screened in two separate screens. The second SSM library had a maximum diversity of 100, and 58,500 mutants were screened in one screen. Both library sizes were estimated by counting colonies generated by transforming the SSM reaction. For ePCR, 18,900 transformants, or 1% of the total library of 1.8×10⁶ (and well below the maximum number of 20²⁷⁶ variants, which is experimentally inaccessible) were screened. Larger mutant libraries may be screened. In a typical screen, over 100 colonies on both the wild-type and library plates were observed in the absence of spectinomycin, and 0-100 colonies were observed on plates that contained spectinomycin (≥400 μg/ml).

The spectinomycin resistance of B2H-containing strains was examined by following these steps: (i) The S1030 cells were transformed with pMBIS and variants of pTS and pB2H (Table 2), plated the transformed cells on LB agar supplemented with antibiotics for plasmid maintenance (50 μg/ml kanamycin, μg/ml carbenicillin, 10 μg/ml tetracycline, and 34 μg/ml chloramphenicol), and grew them overnight (37° C.) (to minimize the influence of mutations outside of the terpene synthase, the terpene synthase gene was recloned from all hits.). (ii) The single colonies were used to inoculate 1-2 mL TB (pH=7.0 supplemented with plasmid antibiotics) and the cells grew overnight (37° C. and 225 RPM). (iii) An aliquot of each culture was diluted in 1:75 in 4.5 mL TB (as above) and this dilution grew to an OD₆₀₀ of 0.3-0.6 (37° C. and 225 RPM). (iv) 500 μM IPTG and 20 mM mevalonate was added to each liquid culture and the induced cultures grew for 20 hours (22° C. and 225 RPM). (v) Fresh TB (no antibiotics) was used to dilute each culture to an OD₆₀₀ of 0.5 unless specified otherwise, and the 5-10 μL of the dilution was plated on LB agar plates (pH=7.0) supplemented, unless otherwise specified, with 20 mM mevalonate, 500 μM iPTG, 20 mL/L glycerol, antibiotics for plasmid maintenance (as above), and with varying concentrations of spectinomycin. (vi) The cells were grown at 22° C. for at least 48-72 hours before photographing them.

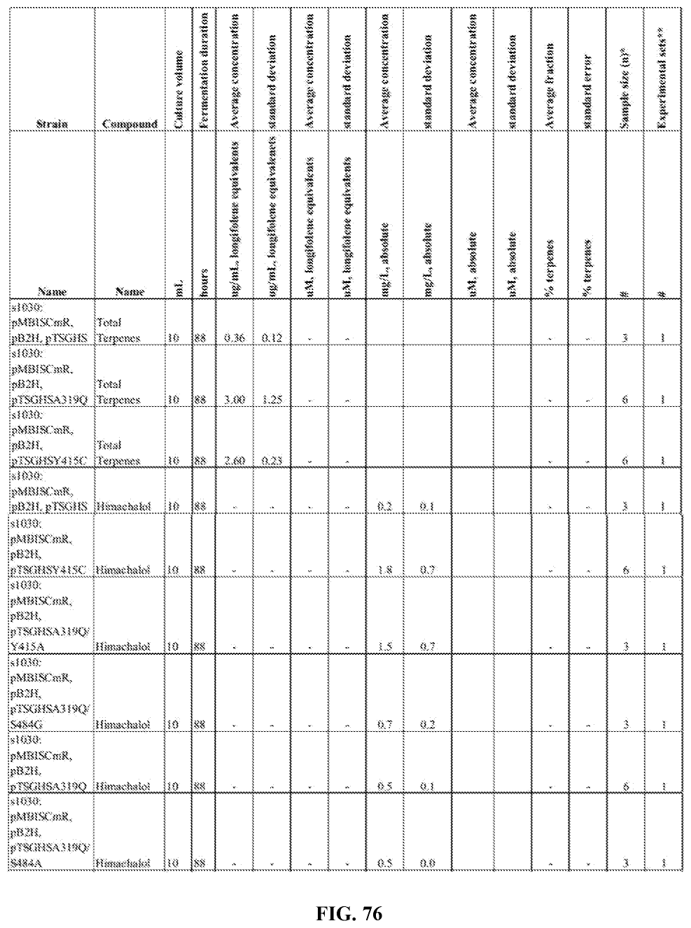

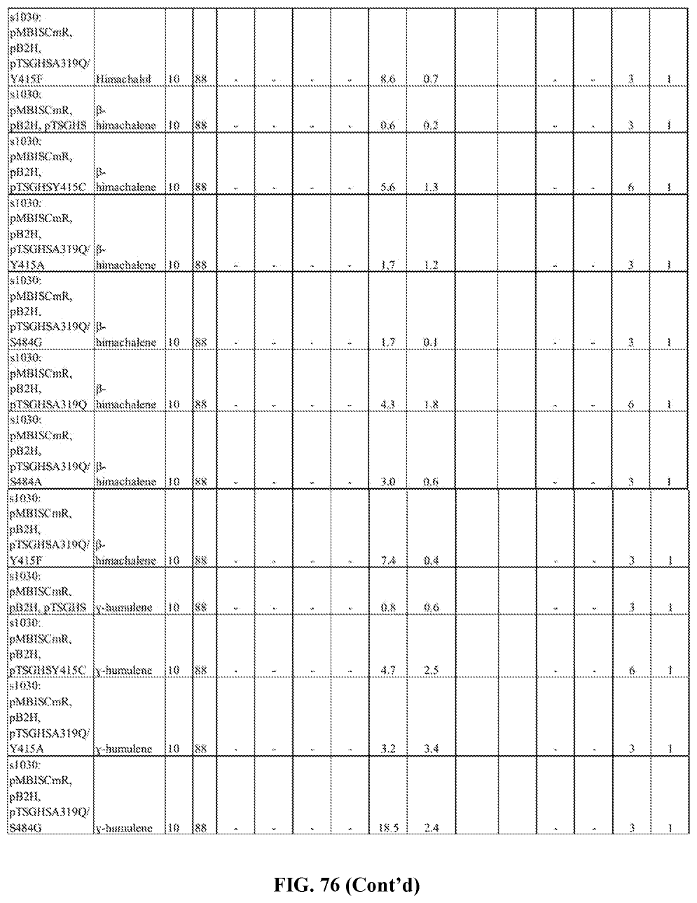

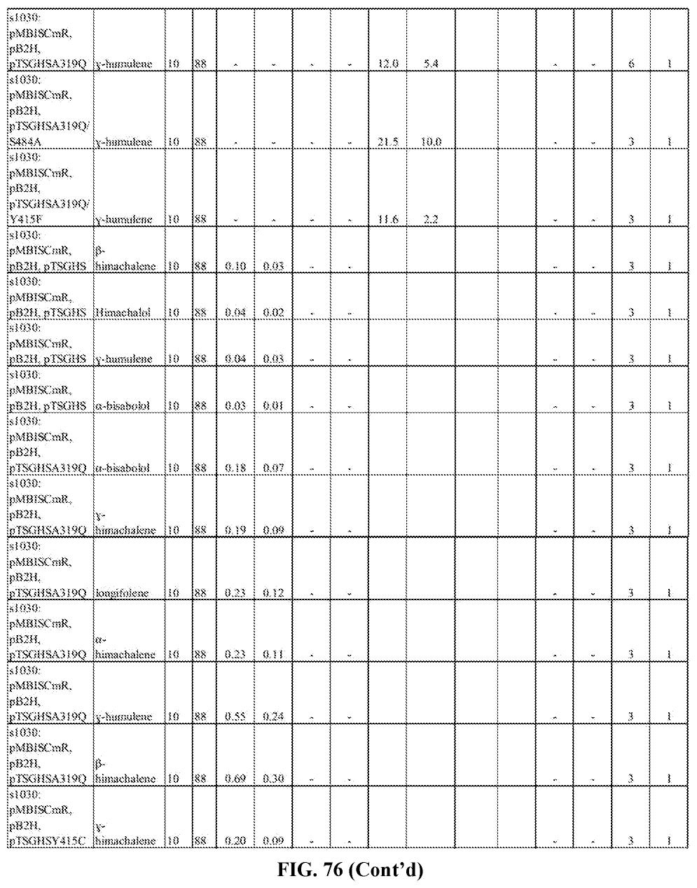

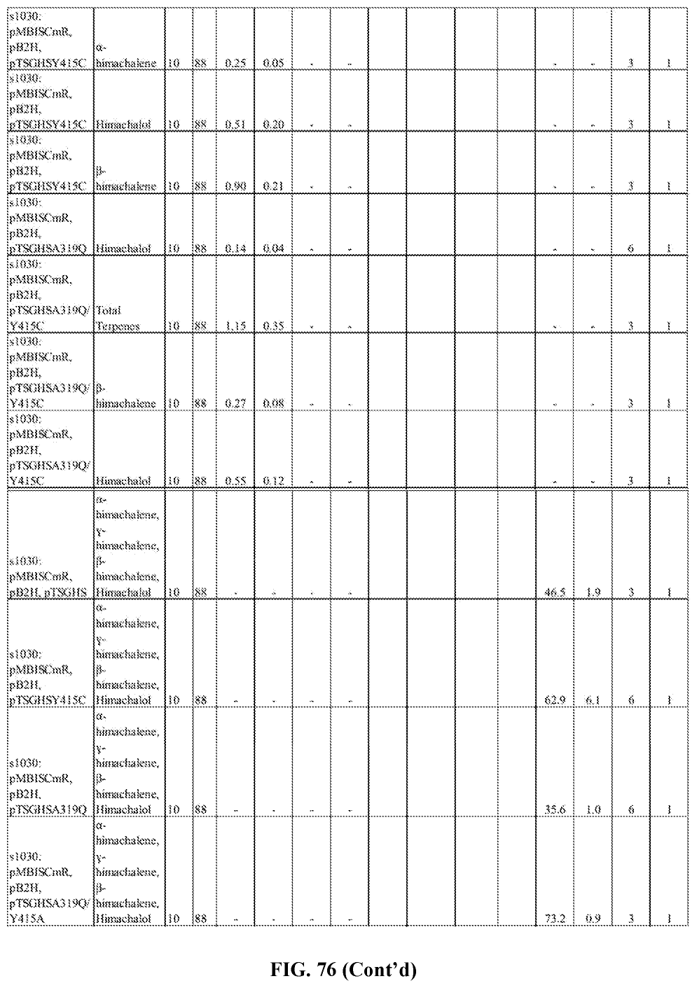

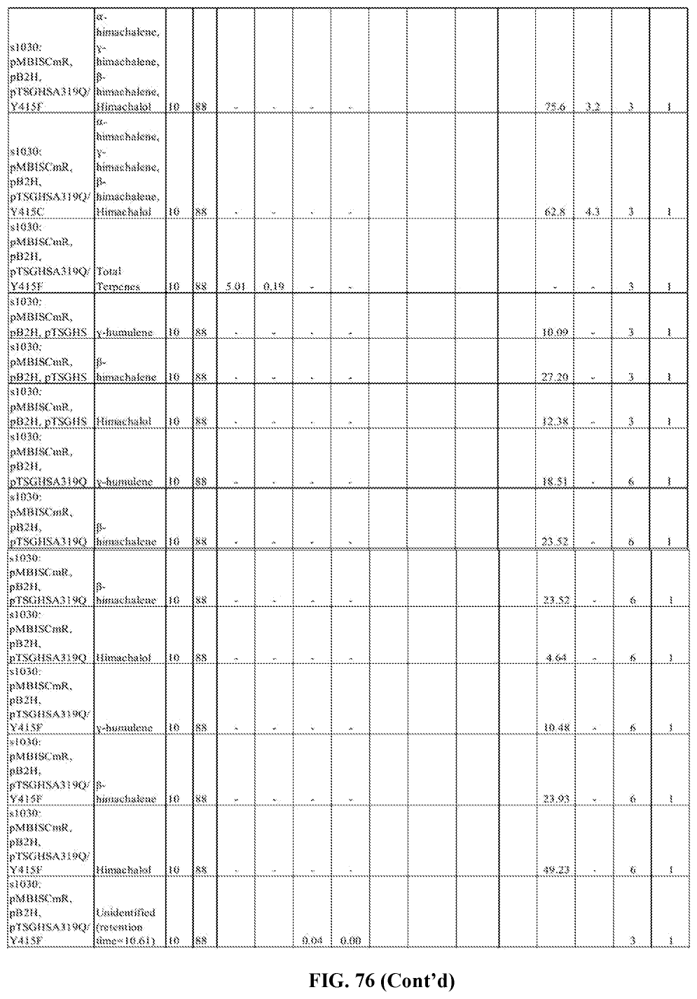

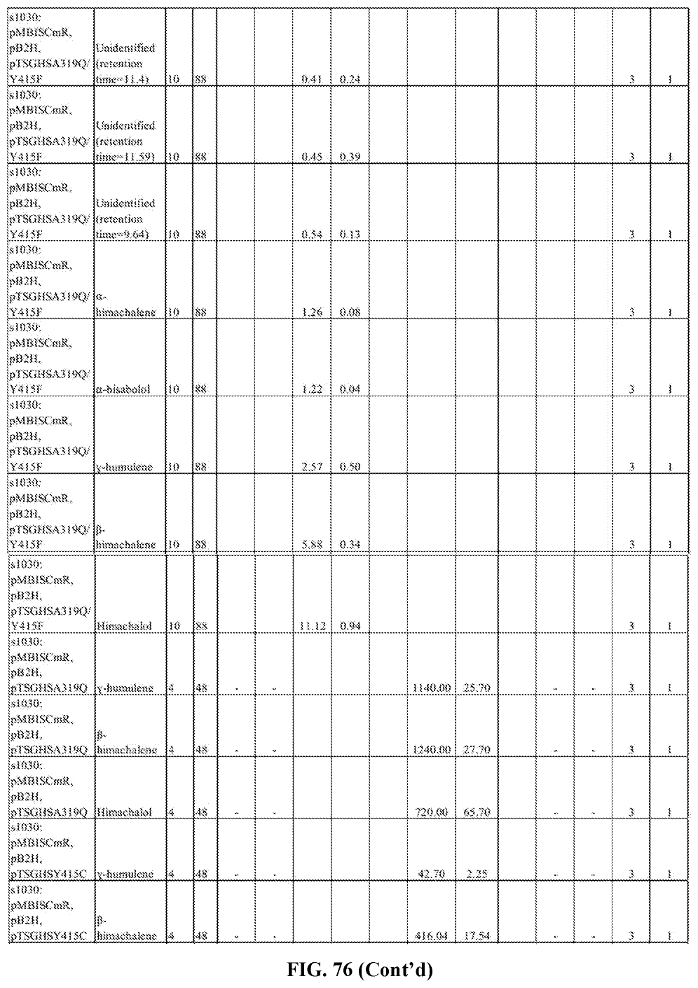

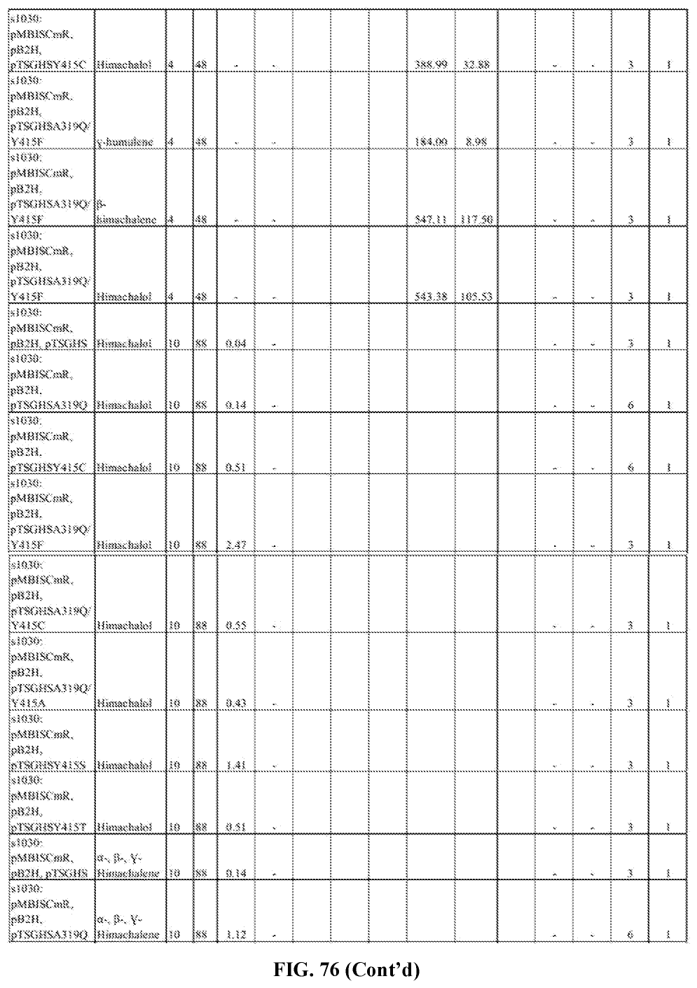

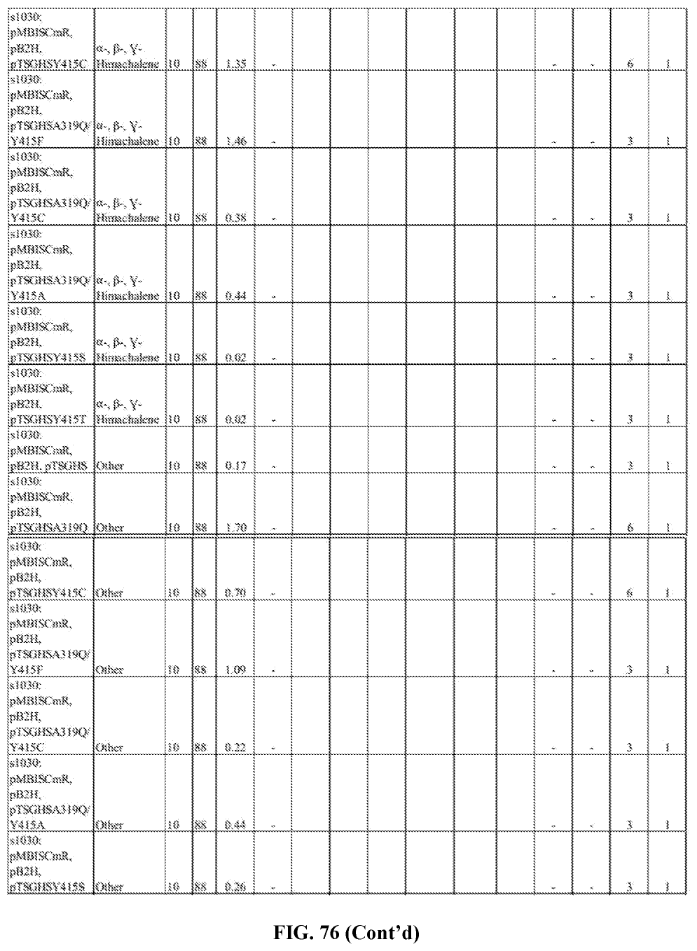

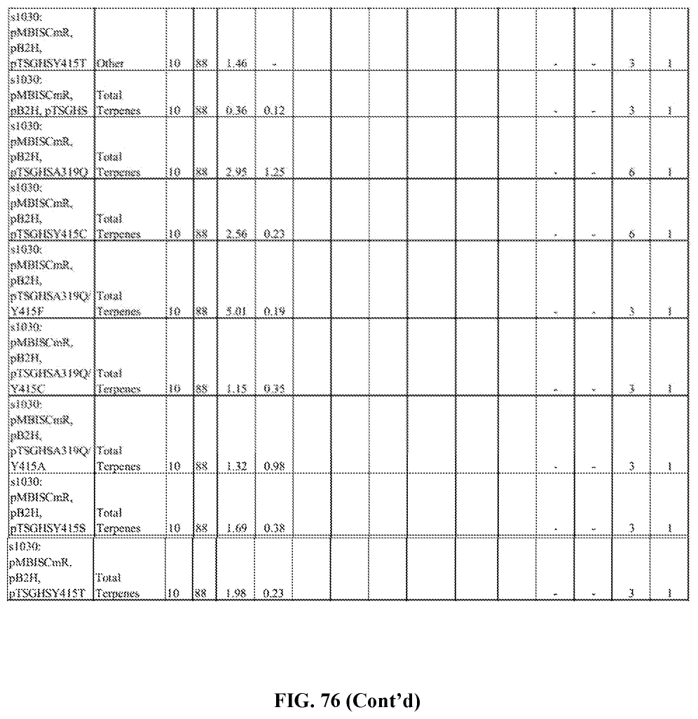

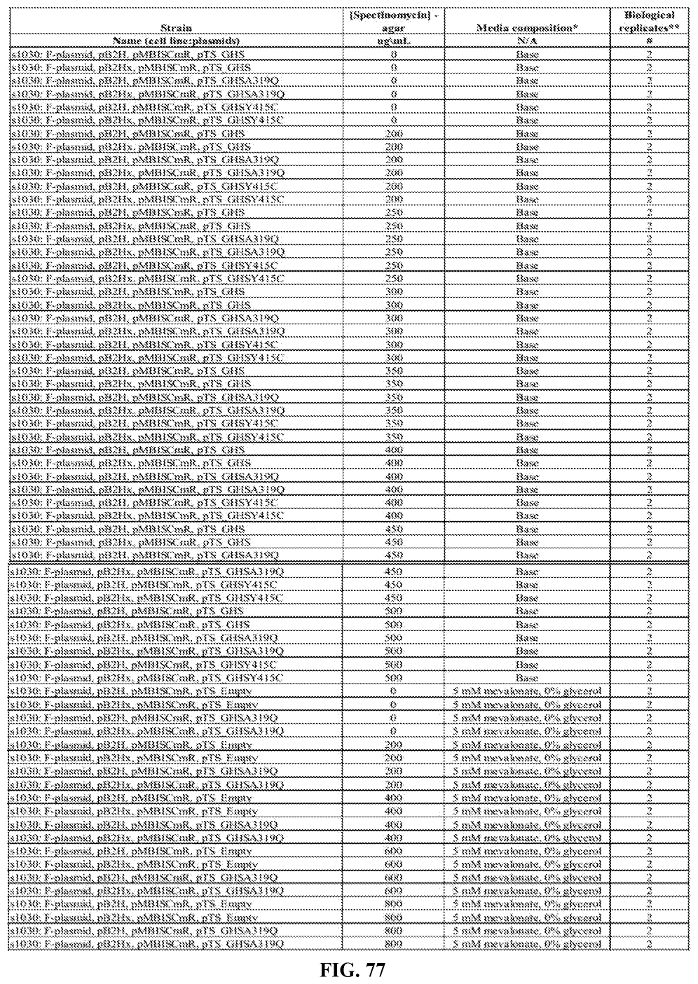

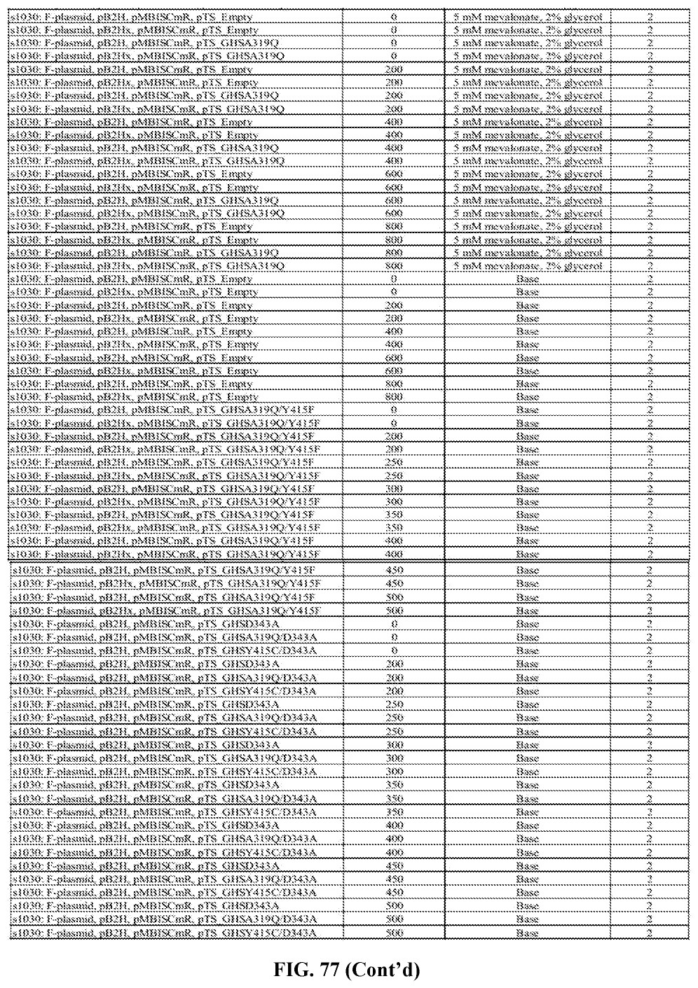

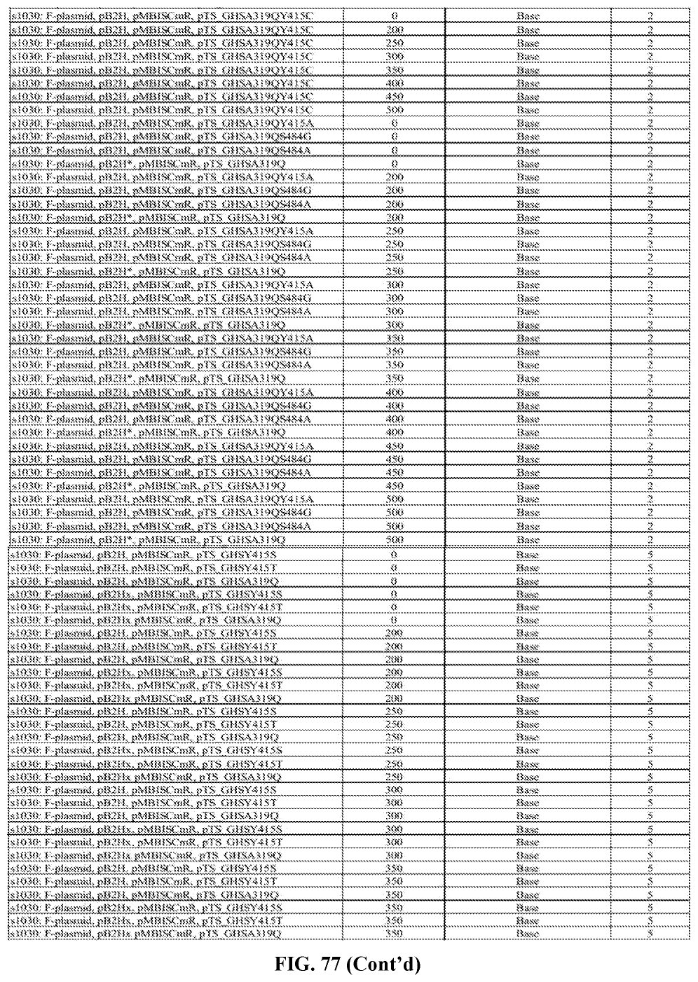

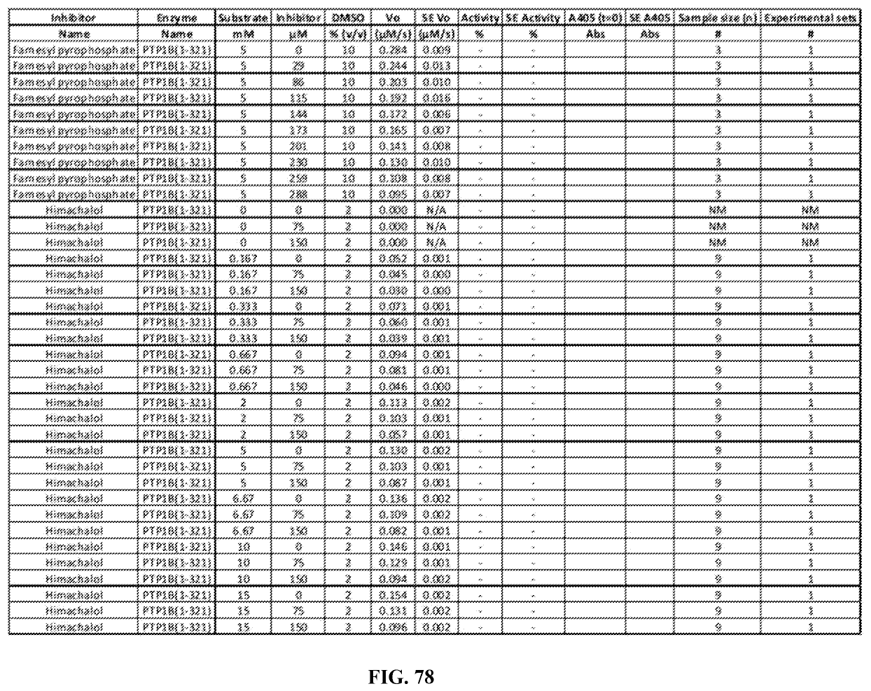

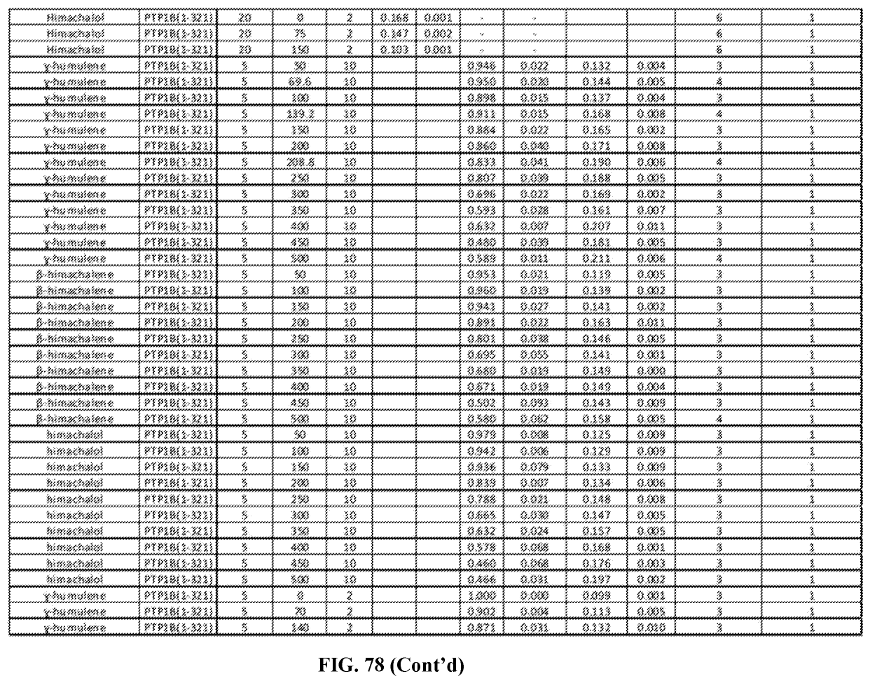

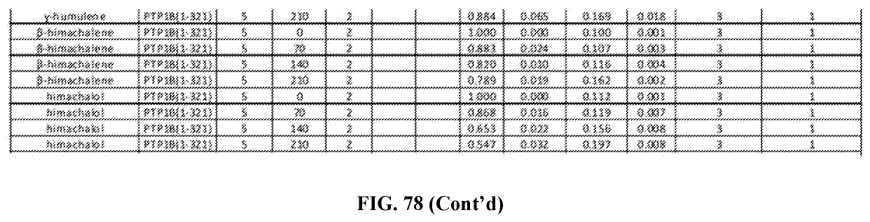

Small-scale terpenoid production was carried in TB (pH=7.0 with plasmid maintenance antibiotics: 50 μg/ml kanamycin, 50 μg/ml carbenicillin, 10 μg/ml tetracycline, and 34 μg/ml chloramphenicol). Briefly, the s1030 cells harboring pMBIS and pB2H were transformed with pTS, the cells were plated on LB agar supplemented with plasmid antibiotics, and grew overnight (37° C.). On the following day, the colonies were picked to inoculate 2 mL of TB (see above), which grew overnight (37° C., 225 RPM). On the subsequent morning, the culture was diluted with TB at a ratio of 1:75 in either 4 mL or 10 mL TB (as above) and grew it to an OD₆₀₀ of 0.3-0.6 (37° C., 225 RPM). After it reached the desired OD₆₀₀, the culture was induced with 20 mM mevalonate and 500 μM iPTG, and grew it at 22° C. for 48-88 hours. FIG. 76 shows exact fermentation times.

DH5α cells were used to carry out large-scale terpenoid production. Briefly, these cells were transformed with pTS and pAM45 (a plasmid that enables mevalonate biosynthesis and conversion to IPP/FPP⁵⁹), plated on LB agar, and grew overnight (37° C.). On the following day, isolated colonies were picked to inoculate 4 mL TB (pH=7.0) supplemented with 20 mL/L glycerol, and grown overnight (37° C., 225 RPM). On the next morning, the culture was diluted with TB at a ratio of 1:50 into Difco TB mix supplemented with 20 ml/L glycerol, and this dilution grew to an OD₆₀₀ of 0.3-0.6 (37° C., 225 RPM). The culture was included by adding 500 μM IPTG and grown at 22° C. for at least 84 hours. Table 2 describes the antibiotics added to LB and TB media for plasmid maintenance.

Hexane was used to extract terpenoids from liquid culture, which varied by culture volume: For 4 mL cultures, 0.6 mL hexane was added to 1.0 mL of culture, vortexed for 3 minutes, centrifuged at 13,300 RPM for 2 minutes, and 0.4 mL of hexane was extracted for analysis. Intracellular terpenoids (always collected from 4 mL cultures) were extracted by: (1) Recording the OD₆₀₀ of each culture at the time of extraction (for determining total intracellular volume per mL of culture) (2) removing 1 mL culture and centrifuging at 4,000×g for 3 minutes (3) discarding the supernatant and adding 100 μL disruptor beads (Chemglass, CLS-1835-BG1)+600 μL hexane (4) and vortexing the bead/hexane mixture for 3 minutes. Samples were centrifuged and stored as before.

For −10 mL cultures, 14 mL of hexane was added to 10 mL of culture, shook at 100 RPM (room temperature) for 30 minutes, transferred to a 50-mL falcon tube, centrifuged at ˜5,000×g for 5-10 minutes, and the hexane layer was removed for analysis.

For large (e.g., 1.0-2.0 L) cultures, hexane was added to 16.7% v/v and mixed by stirring at room temperature for at least 2 hours. The organic layer was recovered with a separation funnel and centrifuged it at 5,000×g for 5-10 minutes. The final hexane layer was removed for further analysis.

Intracellular concentrations of terpenoids were examined by extracting these compounds from cells grown in 4-mL cultures. Briefly, at 48 hours, 1 mL of cell culture was removed, centrifuged for 3 minutes (4000×g), and the supernatant was discarded. Terpenoids were extracted from the cell pellet by adding 600 μL hexane and 100-μL of 0.1-mm disrupter beads (Chemglass, CLS-1835-BG1) and vortexing the suspension for 3 minutes. The resulting lysate was centrifuged at 17,000×g for 2 minutes and the resulting hexane layer was analyzed using GC/MS as described below. Finally, intracellular concentrations of each terpenoid (C_{cell}) was determined per below:

$${C}_{cell} = \frac{{C}_{culture} \cdot {V}_{hexane}}{η \cdot {OD}_{600} \cdot {C}_{OD} \cdot {V}_{cell}}$$

by using the above equation, where C_{culture} is the concentration of terpenoids in the hexane, Vₕₑₓₐₙₑ is 600 μL, η is the extraction efficiency, C_{OD} is the OD-specific cell concentration (8.2×10⁸ cells ml⁻¹ OD⁻¹), and V_{cell} is the volume of a single cell (3.9 fL/cell)⁶⁰. For initial estimates, an extraction efficiency of 1 was assumed, which assumes both complete cell lysis and complete partitioning of terpenoids from the aqueous to the organic layer; accordingly, this approach may underestimate intracellular terpenoid concentrations.

All samples were analyzed with a gas chromatograph/mass spectrometer (GC-MS; a Trace 1310 GC fitted with a TG5-SilMS column and an ISQ 7000 MS; Thermo Fisher Scientific). All samples were prepared by adding 20 μg/ml of methyl abietate as an internal standard, except when estimating purity. When the peak area of the internal standard exceeded ±50% of the average area of all samples containing that standard, the corresponding samples were re-analyzed. For all runs, the following GC method was used: hold at 80° C. (3 min), increase to 250° C. (15° C./min), hold at 250° C. (6 min), increase to 280° C. (30° C./min), and hold at 280° C. (3 min). To identify various analytes, m/z ratios were scanned from 50 to 550. The molecules were identified by using the NIST MS library and, when necessary, confirmed this identification with mass spectra reported in the literature. When displaying chromatograms, the peak for methyl abietate or himachalol was aligned if necessary (due to shifting retention times arising from column trimming carried out as part of routine maintenance). Purity was estimated as the fraction of the total chromatogram area comprised by the peak of interest.

Sesquiterpenes were quantified by using select ion mode (SIM) to scan for the molecular ion (m/z=204) and an ion common to both sesquiterpenes and methyl abietate, the internal standard (m/z=121). The peaks that made up <1% of the total integrated area in the m/z=204 chromatogram were ignored. The remaining peaks were quantified using the common ion m/z=121 and Eq. 3-2, where Aᵢ is the

$${C}_{i} = {C}_{std} * \frac{{A}_{t}}{{A}_{std}} * R \qquad Eq . V - 2$$

$$R = \frac{{A}_{std , o} / {C}_{std , o}}{{A}_{ref , o} / {C}_{ref , o}} \qquad Eq . V - 3$$

area of the peak produced by the analyte i, A_{std} is the area of the peak produced by a standard concentration (C_{std}) of methyl abietate in the sample, and R is the ratio of response factors for longifolene (a commercially available product of GHS) and methyl abietate in a reference sample. Table 5 provides the concentrations of all standards and reference compounds used in this study.

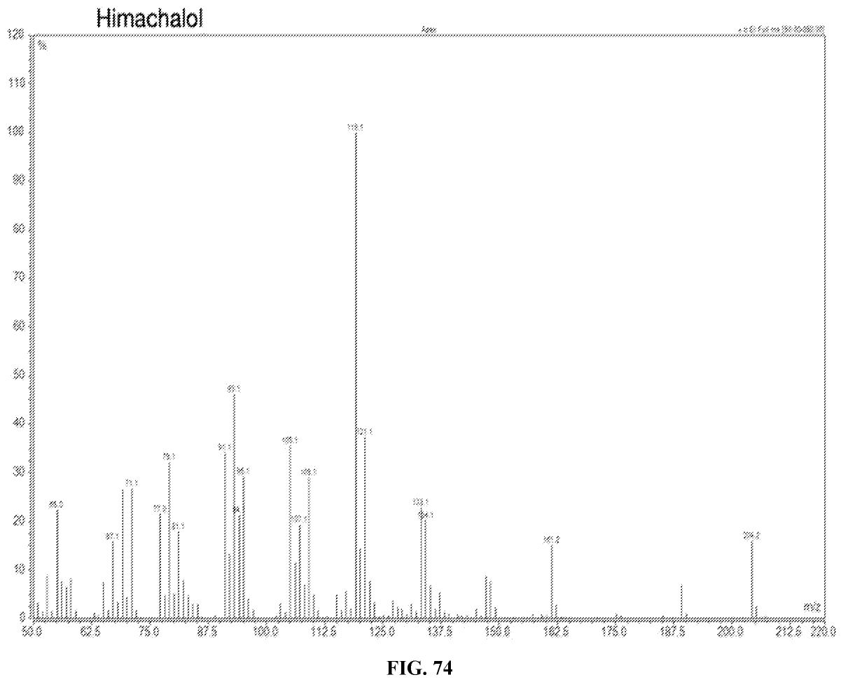

Himachalol was isolated from two 2-L cultures of GHS A319Q/Y415F grown in 4-L Erlenmeyer flasks. Terpenoid biosynthesis and extraction were carried out as described above. The hexane extract was dried to ˜500 μL with a rotary evaporator and dry loaded the sample onto a 12 g C18 column (Biotage Sfar HC Duo). Indole was removed from the terpenoids using C18 chromatography with a Biotage Selekt (5 CVs 70% acetonitrile in water, 5 CV's 85% acetonitrile in water, 5 CV's 100% acetonitrile in water; 10 mL fractions). The terpenoid content of various fractions were checked by using thin layer chromatography (TLC, 3:7 ethyl acetate/hexane) supplemented with a vanillin/sulfuric acid detection method (heating at 125° C. for ˜30 seconds). Himachalol was identified using the NIST MS library (FIG. 74). Indole appeared as an orange spot and himachalol appeared as a purple spot on TLC plates.

Himachalol-containing fractions were pooled and dried using a rotary evaporator, using ethanol to form an azeotrope for removing water. The dried material was resuspended in 200 μL hexane and loaded onto a 5 g silica column (Biotage Sfar HC Duo) for normal phase purification. Using a Biotage Selekt system, the compound of interest was isolated using an isocratic gradient (10% ethyl acetate in hexane), collecting 5 mL fractions. TLC was used with vanillin/sulfuric acid charring to identify himachalol-containing fractions; himachalol appeared on the TLC plates as a purple spot. One 85% pure himachalol fraction (GC/MS) was obtained.

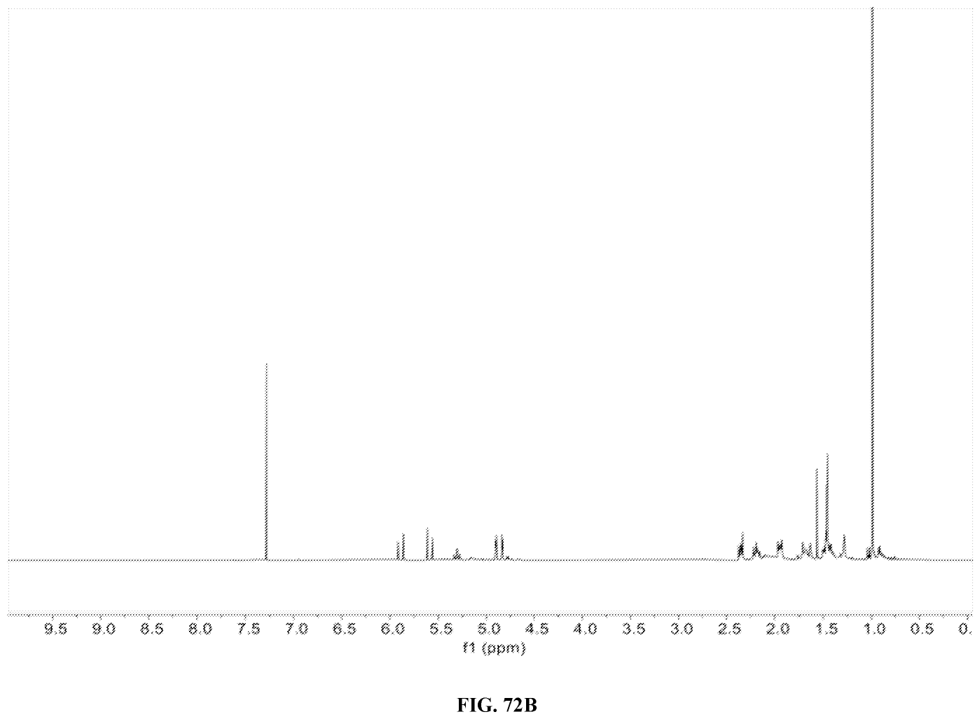

γ-humulene was isolated from two 2-L cultures of GHS A319Q grown in 4-L Erlenmeyer flasks. Terpenoid biosynthesis and extraction were carried out as described above. The hexane extract was dried to ˜500 μL with a rotary evaporator. The material was loaded onto a 5 g silica column (Sigma) and gamma humulene was isolated using vacuum liquid chromatography (isocratic 100% hexane gradient, 3 mL fractions). The terpenoid content of various fractions were checked by using thin layer chromatography (TLC, 3:7 ethyl acetate/hexane) supplemented with a vanillin/sulfuric acid detection method. A single fraction containing >85% pure γ-humulene was obtained. γ-humulene appeared as a purple spot on the TLC plates. The composition of terpenoid-containing fractions were analyzed with GC-MS and, owing to its thermal instability, estimated the purity of γ-humulene using 1H NMR (FIG. 72).

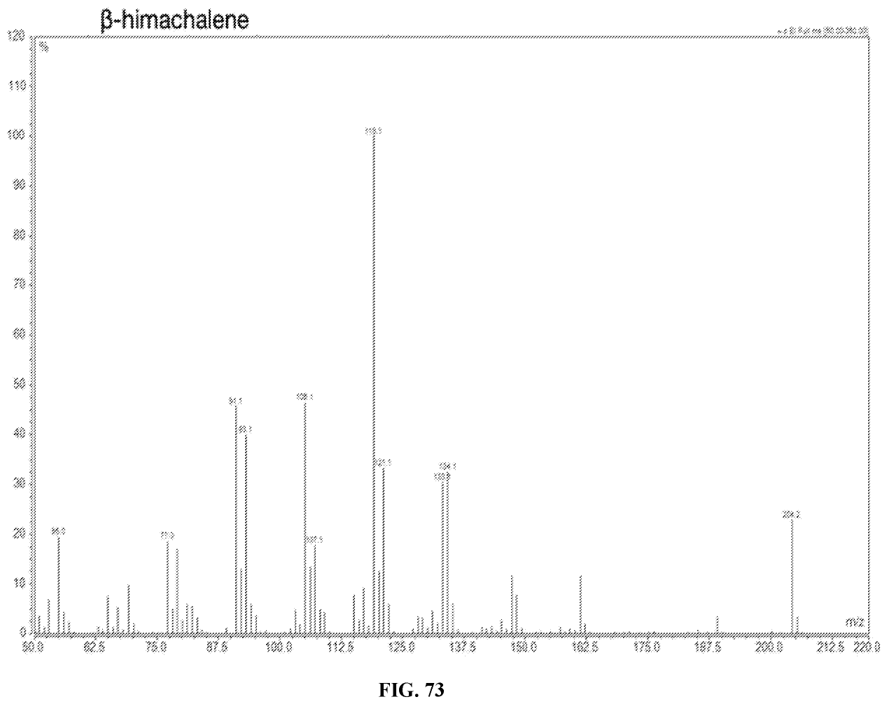

β-himachalene was isolated from cedarwood oil (King Soopers). 502 mg of the oil was loaded onto a 20 g silica column (Sigma) and the non-himachalene components were removed using VLC (10 fractions, 0% ethyl acetate in hexanes; 5 fractions, 5% ethyl acetate in hexanes; 5 fractions, 10% ethyl acetate in hexane; 10 mL fractions). The fractions were analyzed using the vanillin acid-sulfuric acid detection method and GC/MS, obtaining a fraction enriched in α-, β-, and γ-himachalene. β-himachalene was identified using the NIST MS library (FIG. 73). This fraction was dried using a rotary evaporator, and was resuspended in ˜300 μL hexane, and was loaded the resuspended terpenoids onto a 10-g silica column (Biotage Sfar HC Duo). Using a Biotage Selekt system, β-himachalene was isolated using the following gradient: 10 column volumes of 5% ethyl acetate in hexanes, 1 column volume of 5%-10% ethyl acetate in hexanes, 10 column volumes of 10% ethyl acetate in hexanes. The composition of terpenoid-containing fractions were analyzed with GC-MS. The terpenoid content of various fractions were checked by using thin layer chromatography (TLC, 3:7 ethyl acetate/hexane) supplemented with a vanillin/sulfuric acid detection method. β-himachalene appeared as a purple spot on the TLC plates. A single fraction containing 86% pure β-himachalene (GC/MS) was obtained.

PTP1B was purified as described previously. Briefly, the E. coli BL21 (DE3) cells were transformed with a pET21b vector containing the catalytic domain of PTP1B (residues 1-321) modified with a 6× polyhistidine tag on its C-terminus. The cells were grown in 1-L cultures to an OD₆₀₀ of 0.3-0.6 (37° C., 225 RPM), induced with 500 μM IPTG, and grown at 22° C. for 20 hours. The cells were lysed with B-PERII, and purified PTP1B by using desalting, nickel affinity, and anion exchange chromatography (HiPrep 26/10, HisTrap HP, and HiPrep Q HP, respectively; GE Healthcare). The final protein was stored (50 μM) in HEPES buffer (50 mM, pH 7.5, 0.5 mM TCEP) in 20% glycerol at −80° C.

The soluble fraction of several GHS variants were measured by using the Nano-Glo HiBit Lytic Detection System (Promega). E. coli BL21 was transformed with a pET28a vector containing Y415C, A319Q, or A319Q/Y415F with a HiBit tag on the N-terminus. Individual colonies were used to inoculate 2 mL of TB media, which grew overnight (37° C., 225 RPM). Each overnight culture was diluted 1:50 in 4 mL TB in a 24-deep well block and grown (37° C., 225 RPM) to an OD₆₀₀ of 0.5-0.9, at which point 500 μM IPTG was added and the cultures were grown for an additional 24 hours (37° C., 225 RPM).

Following protein expression, 200 μL of each culture was transferred to a 96-deep well block and cells were lysed by adding 200 μL of the HiBit Lytic reagent (prepared and incubated according to the manufacturer's instructions). In preparation for measuring total concentrations of terpene synthase, 100 μL of each lysis reaction was transferred to a 96-well white microplate (Nunc). In preparation for measuring soluble protein concentrations, the remaining volume of each lysed culture was centrifuged (3,000 RPM, 10 minutes) and 100 μL of each supernatant was transferred to the same microplate. For all wells, the luminescent signal of the total and soluble samples were measured with a Spectramax M5 plate reader, and the soluble fraction of terpene synthase was determined by dividing the soluble signal by the total signal.

Inhibition was examined by FPP by measuring its influence on PTP1B-catalyzed hydrolysis of p-nitrophenylphosphate (pNPP). Briefly, 100-μL reactions were prepared comprising 50 nM PTP1B, 0.167-20 mM pNPP, and 75-150 μM terpenoids in 50 mM HEPES (pH=7.3) with 50 mM TCEP, 50 μg/mL BSA, and 2-10% DMSO. The reactions were initiated by adding pNPP, and the production of p-nitrophenol (pNP) was monitored by measuring absorbance at 405 nm every 10 s for 5 min (SpectraMax iD3 plate reader). When necessary, the solubility of the terpenoids in individual wells were assessed by plotting the A405 values for each well—including a no-inhibitor well—in a single read.

Kinetic data was analyzed by using a custom Matlab script supplemented with a user-generated standard curve (e.g., a plot of absorbance at 405 nm vs. pNP concentration in μM, FIG. 15). This script removes datapoints outside of (i) the linear range of the standard curve and/or (ii) the initial rate regime, and it excludes datasets that contain fewer than 10 datapoints after these processing steps. The all datasets were fit using linear regression with Matlab's backslash operator. To determine the half maximal inhibitory concentration (IC₅₀) of himachalol, kinetic models were evaluated by fitting them to standard models of inhibition (e.g., uncompetitive, noncompetitive, uncompetitive, and mixed inhibition). Briefly, the procedure included: (i) fitting an initial-rate measurements of pNPP hydrolysis with and without inhibitors, (ii) using the Akaike's Information Criterion (AIC) to compare the best-fit single parameter model to each alternative single-parameter model and accepting the “best-fit” model when the difference in AIC (Δᵢ) exceeded 5 for all comparisons (when this criterion was not met, it was deemed that two or more single-parameter models may be indistinguishable), and (iii) using an F-test to compare a mixed inhibition model to the best-fit single-parameter model and accepting the mixed model if p<0.05. The IC₅₀ was estimated by using the best-fit kinetic models to determine the concentration of inhibitor required to reduce initial rates of PTP-catalyzed hydrolysis of 20 mM of pNPP by 50%. The MATLAB function “nlparci” was used to determine the confidence intervals of kinetic parameters, and those intervals were propagated to estimate confidence intervals for each IC₅₀.

Inhibition by FPP, a costly reagent, was examined with three modifications of the above assay: (i) pNPP concentration was held constant (5 mM). (ii) The 10% DMSO was replaced with 10% of a mixture of methanol: 10 mM NH₄OH (7:3). (FPP was purchased as a 1.1 mg/mL solution in methanol: 10 mM NH₄OH (7:3)). (ii) IC₅₀ was estimated by using a linear fit to the initial rate data; 95% confidence intervals were propagated on the regression parameters generated using Matlab's “coefCI function”. This approach, which reflects the limited number of measurements afforded by the FPP stock (measurements which include very high and very low initial rates), may result in a greater error than a more standard approach for estimating IC₅₀ (e.g., 4-parameter logistic curves) but nonetheless provides an order of magnitude estimate of potency.

The influence of GHS mutants on E. coli growth were examined by expressing them with pET29b vectors (including a C-terminal Hibit tag: GSSGGSSGVSGWRLFKKIS; Promega). These plasmids were transformed into BL21 cells plated on LB agar supplemented with 50 μg/mL kanamycin and grown overnight at 37° C. The resulting colonies were used to inoculate 2-mL liquid cultures of each transformation (Difco TB mix supplemented with kanamycin), which were grown overnight (37° C. and 225 RPM). The next morning, each culture was diluted 1:100 in 200 μL liquid media (Difco TB mix supplemented with kanamycin and 50 μM) in a clear 96-well plate (Costar flat bottom). Growth curves were measured using a SpectraMax iD3 plate reader (OD₆₀₀, measurements every 15 minutes after 5 seconds of shaking). When analyzing data, wells with OD₆₀₀>0.04 at t=0 were ignored, an indication of cell aggregates.

Specific growth rate was determined by determining the exponential growth region for each curve (e.g., the span of time over which instantaneous growth rate was constant). \

$$ln ( \frac{{OD}_{t}}{{OD}_{t 0}} ) = μ$$

The data was transformed and plotted the data according to the above equation, where OD, is the OD₆₀₀ at time t, ODₜ₀ is the OD₆₀₀ at the beginning of the exponential growth phase, and μ is the specific growth rate. “μ” was determined as the slope of each transformed plot (using the fitlm function in Matlab) and the error in μ was determined from the 95% confidence intervals for each slope (using the coefCI function in Matlab).

Statistical significance was determined with a one-tailed Welch's t-test (Table 18), and an F-test was used to compare one- and two-parameter models of inhibition (Table 17).

### Example 2: Bacterial Two-Hybrid Systems for the Discovery of Viral Protease Inhibitors

All drug discovery efforts begin by identifying functional molecules. Many small-molecule discovery programs rely on expensive and laborious high-throughput screens of large compound libraries; in contrast, biological systems (e.g., the natural world) are constantly discovering functional molecules through natural selection. Discovery approaches that emulate nature by introducing genetically encoded selection pressures into microbes that can produce structurally diverse compounds could be useful for the efficient discovery of novel molecules with pharmaceutically relevant activities. This study used a bacterial two-hybrid architecture to encode selection pressures that gene expression to the activity of important drug targets: HIV-1 protease (HIV-1Pr) and 3-chymotrypsin-like protease (3ClPro) from SARSCOV2. The bacterial two-hybrid architecture identified differences in the optimal design of each protease system and present a workflow that should be adaptable to the development of similar tools. The bacterial two-hybrid architecture screened each protease B2H against 74 terpenoid pathways and identified several enzyme combinations that show altered resistance phenotypes (implying biosynthesis of protease inhibitors). These results expand on the early work by showing that bacterial two-hybrid systems enable the detection of biosynthetically accessible small molecules that inhibit proteases and, more broadly, suggest that these systems provide a particularly versatile means of screening biosynthetic pathways that produce medicinally relevant natural products.

Nature is replete with enzymes that produce an enormous variety of biologically active molecules. Over millennia of evolution, compounds carrying out specific biological activities (pheromones, pest repellants, toxins, etc.) have been enriched—or “discovered”—through selective pressures. Many of these natural products exhibit useful medicinal activities in humans, but these properties are often discovered serendipitously or through screens of chemical libraries. Unfortunately, these screens usually require compound isolation from natural sources—a laborious and expensive endeavor. Microbial systems have excelled at producing terpenoids, alkaloids, peptides, and other natural products in laboratories; however, identifying functionally valuable molecules still requires non-trivial purification schemes followed by in vitro assays. Consequently, microbial systems producing drug-like molecules have been limited to the production of single compounds with known value (e.g. the pharmaceutical precursors dihydroartemisinic acid or taxadiene) or many diverse compounds that lack functional characterization.

Genetically encoded systems that connect the activity of compounds in a cell to easily measurable outputs (e.g. fluorescence, luminescence, or growth) could be useful for purification-free, functional characterization of microbially-produced molecules. Unfortunately, linking the activity of a compound to transcription of such signals is not straightforward, especially if the desired activity is modulating a drug target completely orthogonal to microbial gene expression. A limited number of systems have been developed to respond to a biosynthesized molecule's activity against a medicinally relevant enzyme in a cell (e.g. rho bacterial termination factor, HIV-1 protease, and protein tyrosine phosphatase 1B), but these strategies have not been generalized to other targets or used with large biosynthetic libraries (e.g. >50 pathways).

In this work, the bacterial two-hybrid architecture expanded on the previously reported phosphatase-based system by developing genetically encoded bacterial two-hybrids that respond to the activity of HIV1-Pr and 3ClPro. To date, the associated viruses (HIV and SARS-COV-2) are responsible for >40 million deaths worldwide. While no 3ClPro inhibitors are approved for use today, 10 HIV-1Pr inhibitors have been. Even so, resistance to HIV-1Pr drugs frequently emerges (especially in the developing world) and they often require suboptimal dosing/delivery strategies due to their poor pharmacokinetic properties. Thus, new inhibitors of both enzymes could be useful for drug development. To screen for such molecules, the bacterial two-hybrid architecture developed and optimized luminescent systems responding to the activity of each protease and used the best constructs to inform the design of growth-coupled systems. The bacterial two-hybrid architecture used these tools to screen >100 metabolic pathway/inhibitor targets with a simple drop-plating assay, allowing us to quickly identify pathways producing molecules with different survival phenotypes alongside each protease B2H (implying varying levels of inhibitory activity.) The findings suggest that these tools can quickly screen biosynthetic pathways for molecules with broad or specific inhibitory activities through parallel screens of two-hybrid systems harboring different drug targets. Coupled with large biosynthetic pathway libraries, these designs should prove useful in the discovery of novel viral protease inhibitors.

FIG. 5A shows General architecture for a protease-inhibited bacterial two-hybrid system. Components include (i) a phosphotyrosine substrate (MidT) fused to the omega subunit of RNA polymerase or portions thereof (RpoZ) with a linker containing a protease cleavage site (CS), (ii) a superbinder Src homology 2 domain (SH2) fused to a DNA-binding protein (cI), (iii) a kinase (cSrc) and a chaperone to aid in kinase folding (CDC37), (iv) a protease, (v) an optimized two-hybrid promoter (pLacZopt) driving expression of a gene of interest (GOI), and (vi) binding sites for RNA polymerase (RNAP) and cI (cI op). Src kinase phosphorylates MidT, enabling binding to SH2 and localization of RNAP to drive transcription of the GOI. An active protease should cleave the MidT/RpoZ fusion, preventing this localization and, thus, GOI expression. The introduced proteases HIV-1Pr and 3CLpro with computationally designed RBSs, the indicated cleavage sites in the MidT/RpoZ linker, and a spectinomycin resistance gene (aadA, “denoted as SpecR”). “*” indicates an inactive protease (HIV1-PR: D25N mutation, 3ClPro: H41A mutation).

To screen metabolic pathways for protease inhibitors, a genetically encoded system that links protease activity to a selective pressure was sought. To this end, a bacterial two-hybrid (B2H) system was developed to control the expression of an essential gene. A system was previously created in which MidT (a phosphotyrosine substrate) and a superbinder v-Src SH2 domain were fused to the omega subunit of RNA polymerase or portions thereof (RpoZ) and a DNA-binding cI repressor protein, respectively. Adding Src kinase phosphorylates the substrate, enabling binding to the SH2 domain, localization of RNA polymerase, and transcription of an antibiotic resistance gene from an optimized B2H promoter, pLacZopt. It was hypothesized that cleavage sites could be encoded in the MidT-RpoZ linker to make a protease-responsive B2H: active protease would cleave the fusion, preventing RpoZ from localizing RNA polymerase, and protease inactivation would restore localization and, thus, transcription (FIGS. 5A-5C). This design should be compatible with multiple output signals (e.g. luminescence-which could be useful for rapid characterization of system performance-instead of resistance) as it can control transcription of any gene by using protein fusions with flexible linkers that should readily accept insertions. Prior systems for detecting in vivo activity of heterologous proteases in E. coli relied on essential proteins compatible with inserted cleavage sequences and, thus, could only be used with growth-coupled assays.

In some embodiments, a protease recognition sequence was added to the MidT-RpoZ linker. The protease recognition sequences reduced luminescence but maintained a 4 to 5-fold dynamic range. In those embodiments, E. coli was transformed with a protease induction system and a bacterial 2-hybrid system modified with an inactive PTP1B and a protease-specific cleavage site, which allowed for monitoring of changes caused by protease expression. Monitoring the changes indicated that two HIVpro systems and one 3CLpro system exhibited a decrease in luminescence in response to protease expression, and inactive proteases showed a small decrease in luminescence, which may have been an effect resulting from weak substrate binding and/or a general cellular stress response to protease overexpression. Additional proteases and protease-specific cleavage sites were then screened by adding recognition sites for the papin-like protease of SARS-COV-2 (PLpro), the NS2B/NS3 proteases of the West Nile and Dengue Viruses, WNVpro and DVpro, respectively, and ubiquitin-specific protease 7 (USP7). The proteases and protease-specific cleavage sites were screened alongside bacterial 20 hybrid systems. For USP7, a catalytic domain with and without a C-terminal extension required for activity was included. As a result of the screen, 3CLpro reduced luminescence for multiple recognition sites, indicating that one or more components of the underlying bacterial 2-hybrid system contained a cleavage site for 3CLpro, which was later confirmed to be a site in RpoZ.

In some embodiments, new bacterial 2-hybrid systems promoting spectinomycin resistance were creating. To build survival-modulating systems for 3CLpro and HIVpro, earlier bacterial 2-hybrid systems including a gene for spectinomycin resistance were changed by swapping a protease with PTP1B and adding the best-performing cleavage site from previous screens.

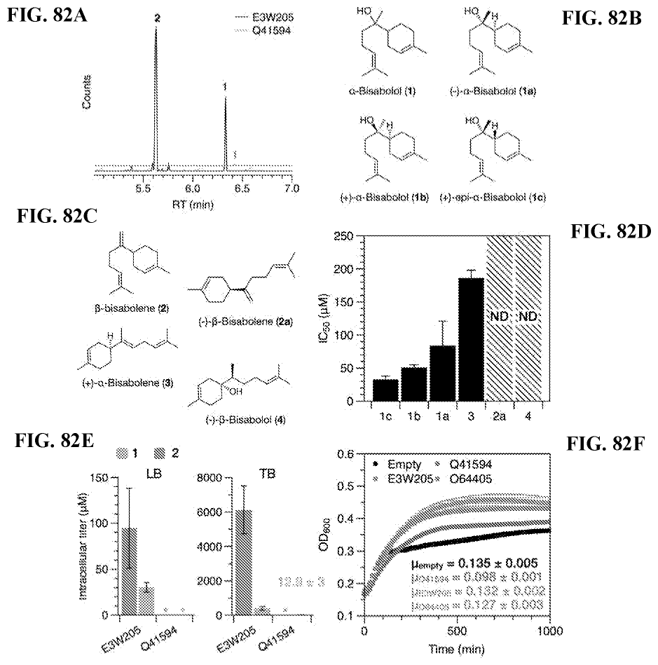

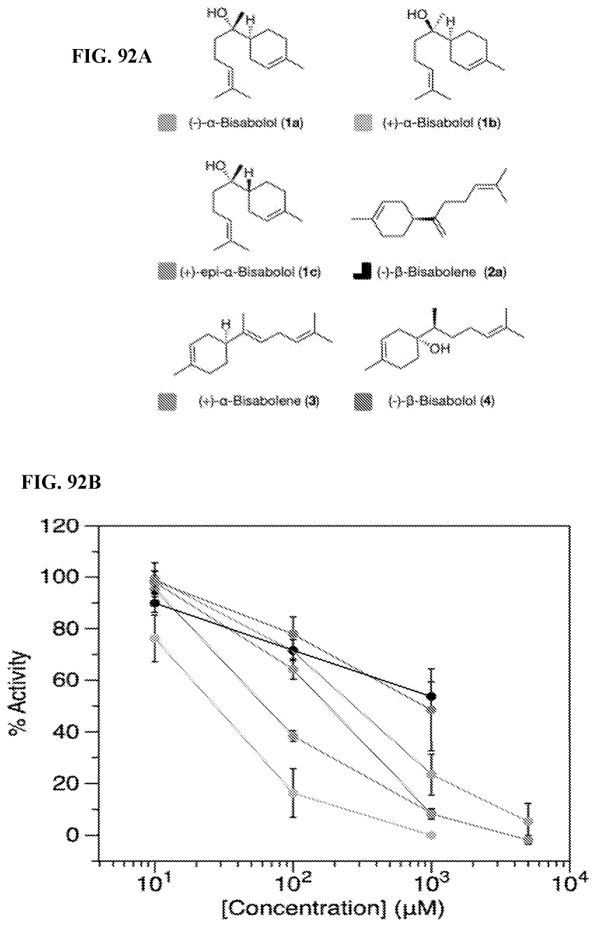

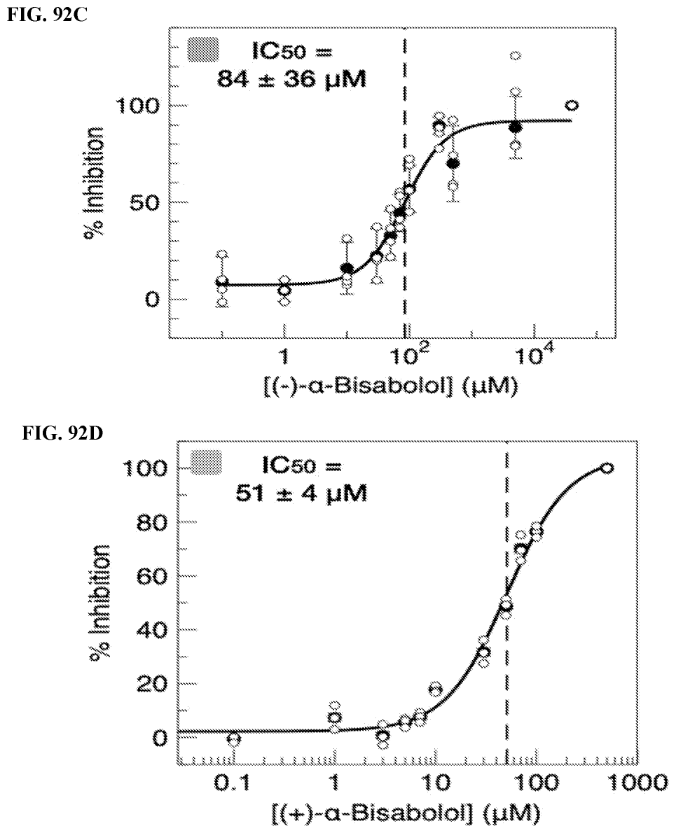

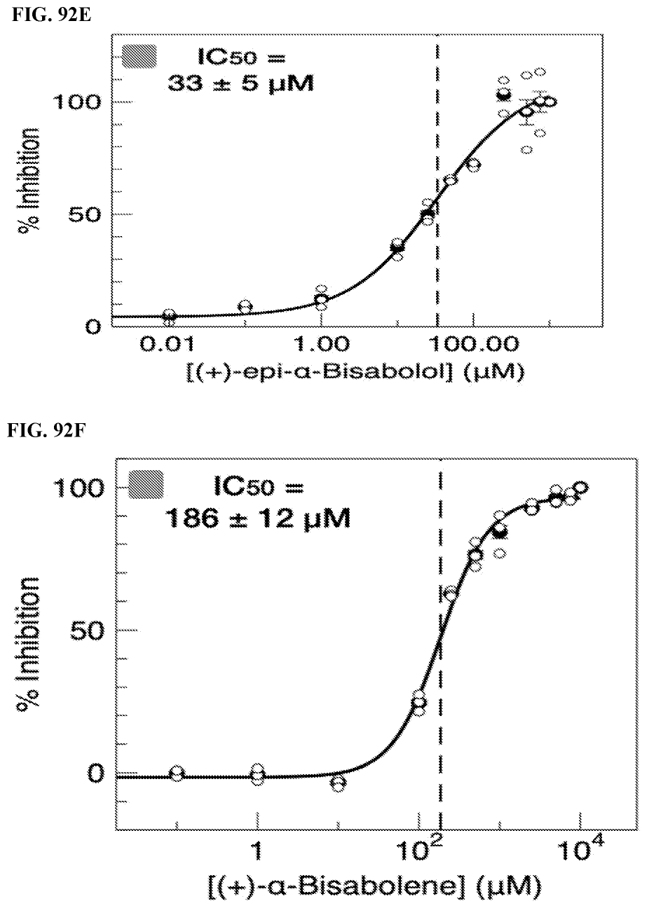

A kinetic characterization of α-bisabolol based on the embodiments described above suggests that α-bisabolol is an inhibitor of 3CLpro. FIG. 82D and FIGS. 92A-92D depict α-bisabolol's inhibition of 3CLpro activity on a model substrate, a fluorogenic peptide. α-bisabolol was particularly interesting for multiple reasons. First, α-bisabolol's IC50 is reasonably good (IC50=30±5 μM to 80±37 μM), relative to the IC50s of lead compounds identified in a previously reported screen of 10,000 compounds—including approved drugs, drug candidates in clinical trials, and other pharmacologically active compounds. Second, kinetic data suggests that α-bisabolol is an inhibitor, and its mode of inhibition could be valuable for building broad-spectrum antivirals for coronavirus diseases. Third, α-bisabolol is particularly small and has a high ligand efficiency.

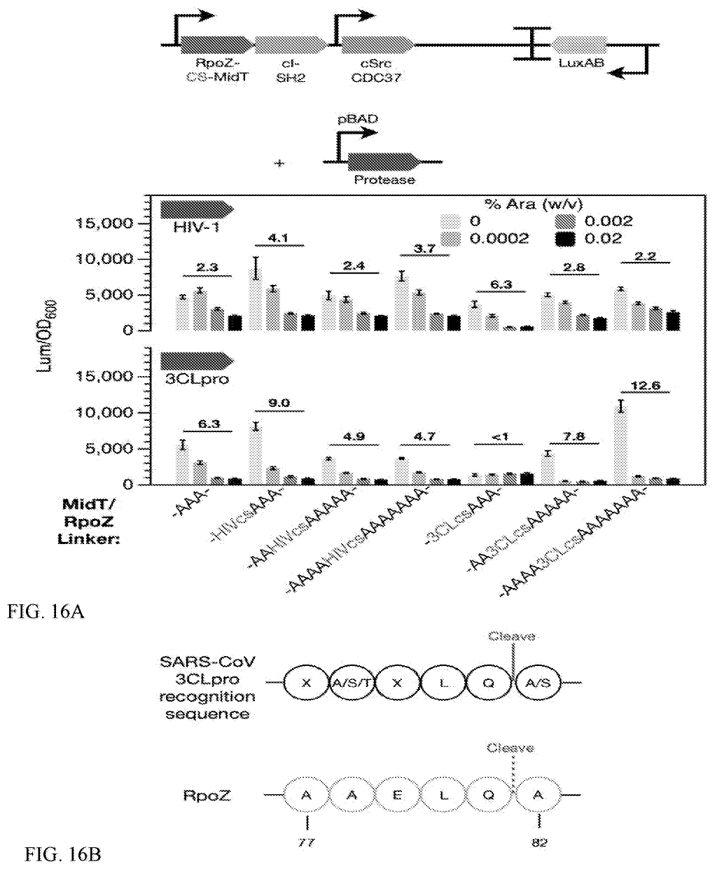

FIGS. 16A-16B show effect of protease-recognition substrate combinations on B2H performance. The B2H systems was created using LuxAB as a reporter gene and the MidT/RpoZ linkers shown and introduced each protease through arabinose induction from a pBAD promoter. HIVcs=KARVLAEAM and 3CLcs=AVLQSFGR. Labels above bars indicate fold-change in luminescence (0% arabinose vs 0.02% arabinose). A potential 3CLpro cleavage site in RpoZ. The black sequence shows a consensus recognition sequence for SARS-COV 3CLpro (96% sequence identity to SARS-COV2 3CLpro) and the blue sequence shows a similar region of RpoZ. The dashed line indicates a potential SARS-COV2 3CLpro cleavage site in RpoZ. RpoZ residues 77 and 82 are labeled. Similar analyses for HIV1-Pr are complicated by the broad range of sequences it can cleave. Error bars denote standard error of n=6 biological replicates.

To begin, the B2H systems that respond to the activity of HIV-1 protease (HIV-1Pr) or SARS-COV-2's 3-chymotrypsin like protease (3ClPro) were created. A cleavage sequence was inserted for each protease into the MidT/RpoZ linker, testing constructs with different numbers of alanine residues around the insertion. Designs lacking cleavage sequences were also tested. To measure transcription from the B2H promoter with a wide range of protease expression levels, HIV-1Pr or 3CLpro were introduced on an arabinose-inducible plasmid and placed a luciferase gene, LuxAB, under control of the B2H promoter (FIGS. 16A-16B). In both cases the optimal constructs (e.g. those with a high luminescent signal in the absence of protease and large reduction in luminescent signal in the presence of protease) included the expected recognition sites and resulted in 4-fold (HIV-1Pr) to 12-fold (3CLpro) reductions in transcription following protease induction.

3CLpro showed significant reductions in luminescence (6-fold) even in the absence of a cleavage sequence in the MidT/RpoZ linker (FIGS. 16A-16B). Comparison of the consensus recognition sequence for SARS-COV 3CLpro (96% sequence identity to SARS-COV2 3CLpro) with the B2H components revealed a potential cleavage site near the C-terminus of RpoZ (FIGS. 16A-16B). Proteolysis at this site would explain the reduction in luminescence observed without a protease recognition sequence. This prompted us to screen each protease against linkers containing non-cognate recognition sites (e.g. HIV1-Pr cleavage sequence against 3CLpro protease, FIGS. 16A-16B). As expected, 3CLpro showed high reductions in luminescence (>4-fold) for all linkers containing the HIV sequence, confirming that 3CLpro can reduce GOI expression independent of the added 3CLpro-specific cleavage site. The largest change in signal was still obtained using a linker containing the 3CLpro sequence; thus, the B2H system included this cleavage site in the development of a selection system.

When using non-cognate or no recognition sites, HIV1-Pr showed smaller reductions in luminescence (2-3-fold). This effect could be consistent with low-level proteolysis; unfortunately, HIV-1Pr can act on a broad range of recognition sequences, precluding simple predictions of cleavage sites in the system. One exception to this trend was a 6-fold change observed with the 3CLpro cleavage sequence and a three-alanine linker. Although the observed effect on transcription was high in this system, the basal signal (e.g., without arabinose induction) was lower compared to the HIV-1Pr plus three-alanine linker. Therefore, the HIV1-Pr site was chosen to be used in the growth-coupled system, hypothesizing that it would afford better survival characteristics due to higher expression of an essential gene in the absence of an active protease.

Next, B2H systems were created that are compatible with selection. The procedure (i) introduced each protease and the best performing linker identified in the luminescence screen into the B2H system; and (ii) introduced aadA (indicated as “SpecR”), a gene encoding resistance to the antibiotic spectinomycin, in place of the LuxAB. The arabinose screen suggested high levels of protease would be important for maximal reduction in aadA expression, so the procedure tested multiple ribosome binding sites (RBS) to achieve high translation initiation rates (TIR) of each protease (FIGS. 5A-5C).

The RBS Calculator was used to design sequences with a wide range of predicted TIR's for each protease, at least 2 of which were tested with each system. To confirm functionality, both WT and inactive enzymes (D25N mutation in HIV-1 Pr, H41A mutation in 3CLpro) were tested. These systems were plated on solid media containing spectinomycin and identified RBS's with TIR's of 20,000 (HIV-1Pr) and 90,000 (3CLpro) that showed poor growth when the proteases were active and robust growth when they were inactive. In agreement with the observed fold-changes with the luminescent system, the B2H systems saw more striking growth differences with 3ClPro than with HIV1-Pr. The B2H designs were used to screen metabolic pathways.

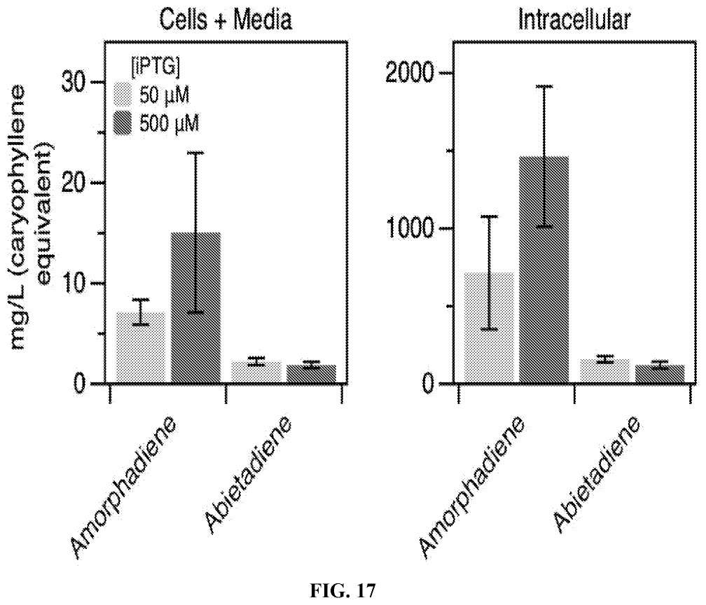

FIG. 17 shows measurements of terpene production in s1030 cells harboring pIUP+FPPS or GGPPS, pB2Hopt₄₀₅, and pTS_Q9AR04 (amorphadiene synthase) or pTS_O64405 (abietadiene synthase). For simplicity, only the major product titers are shown. Error bars denote standard deviation of at least 3 biological replicates.

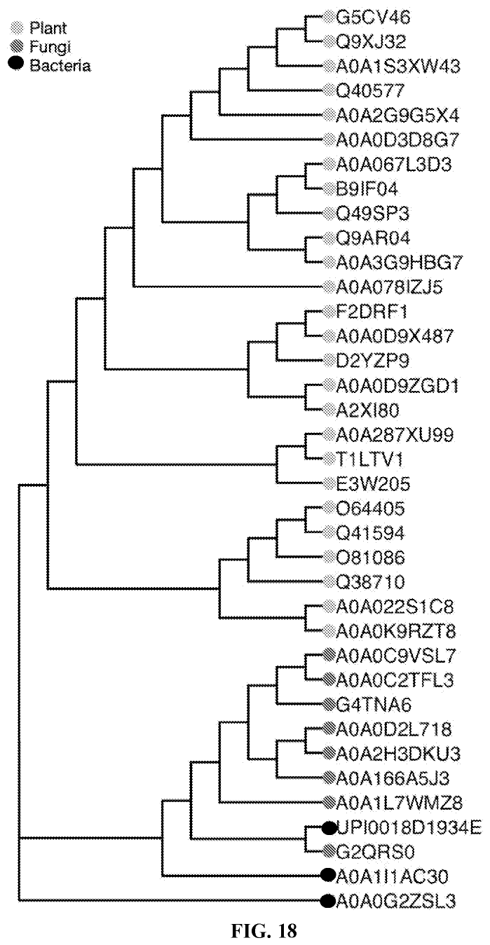

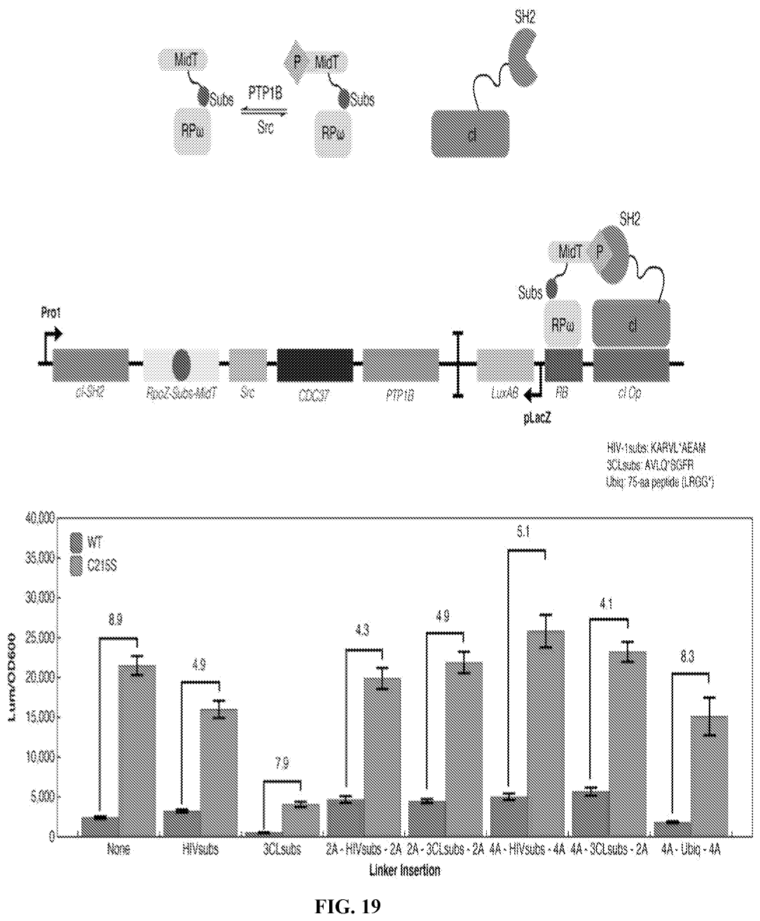

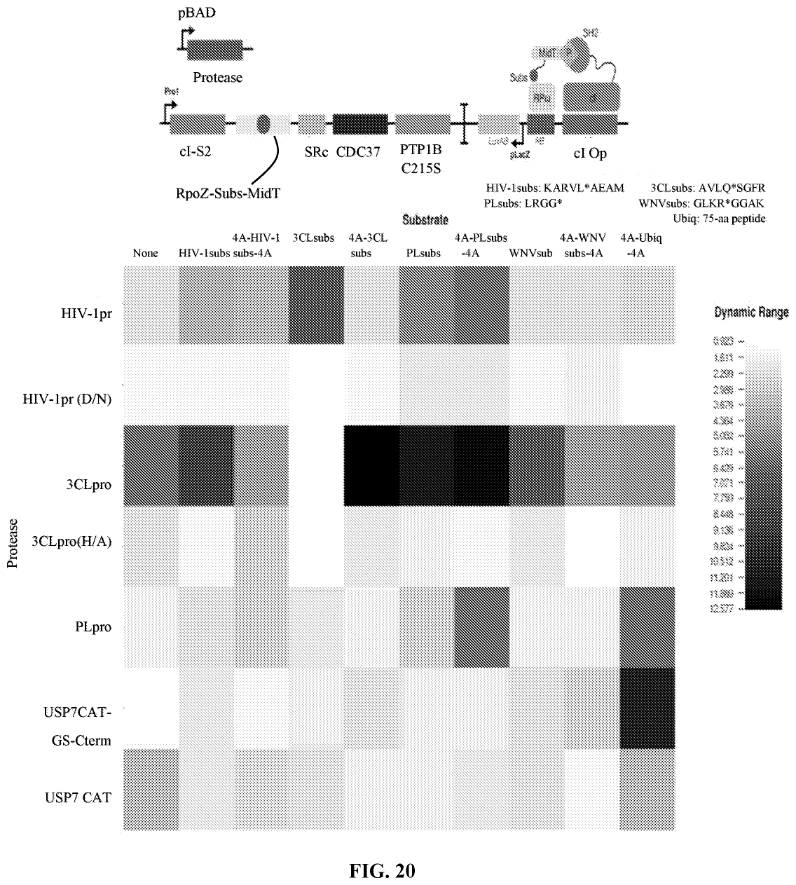

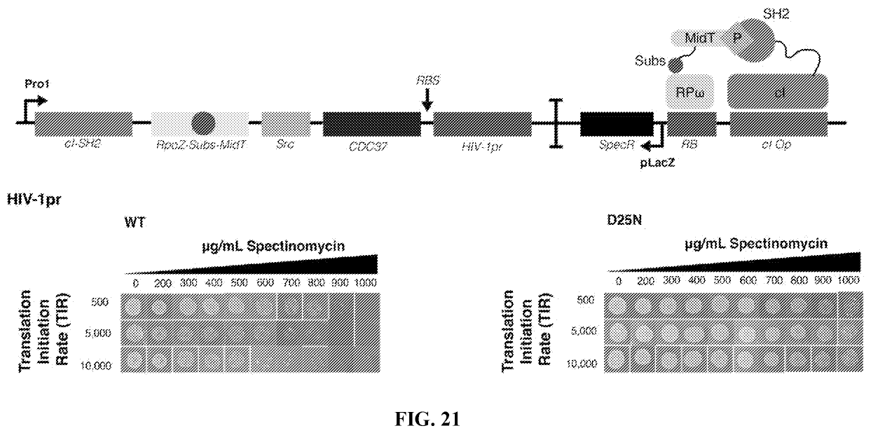

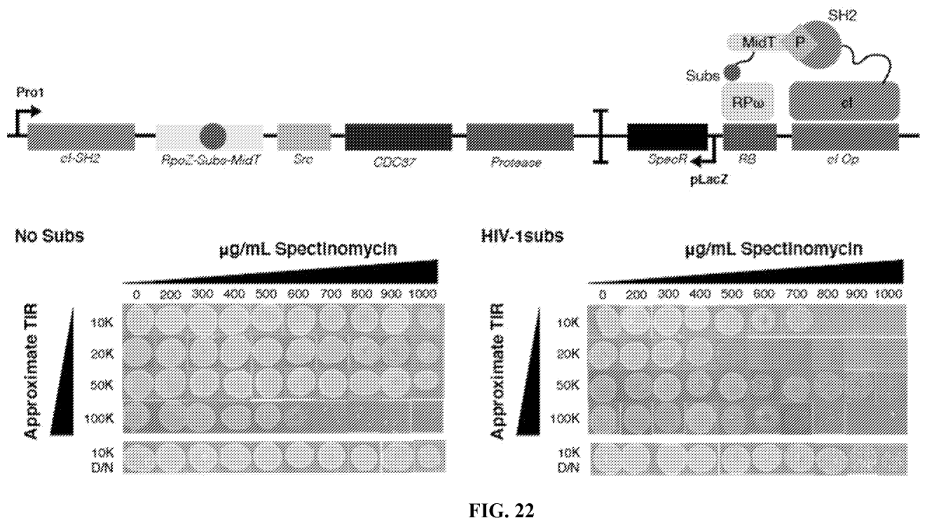

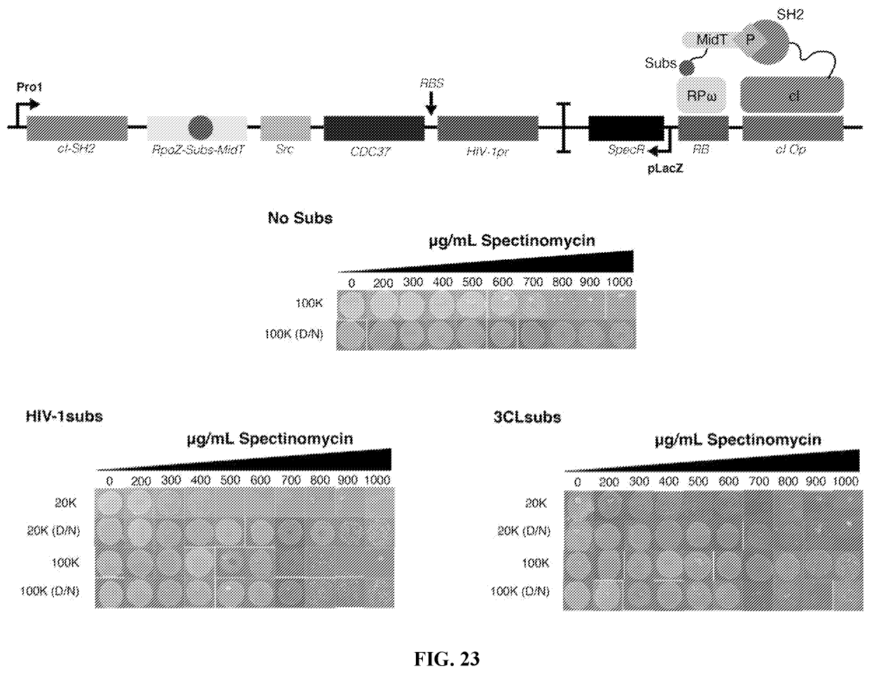

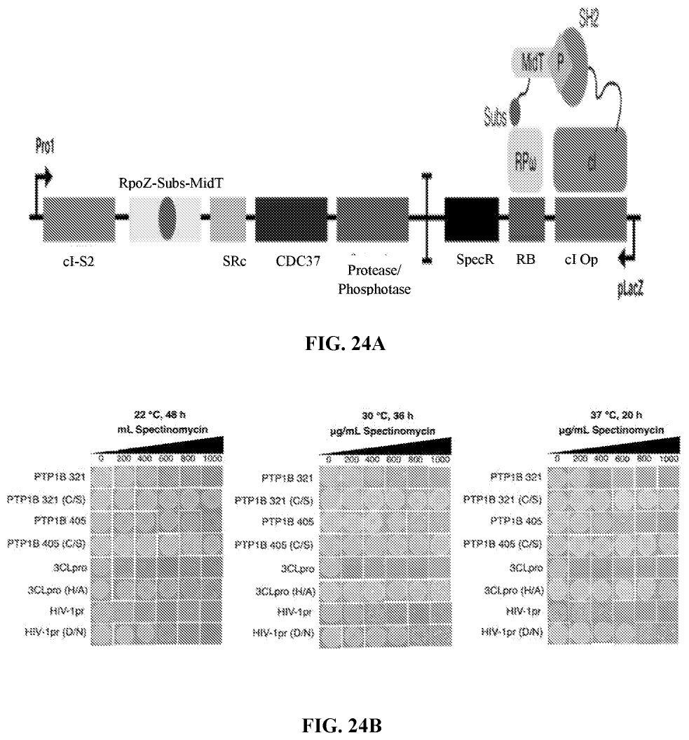

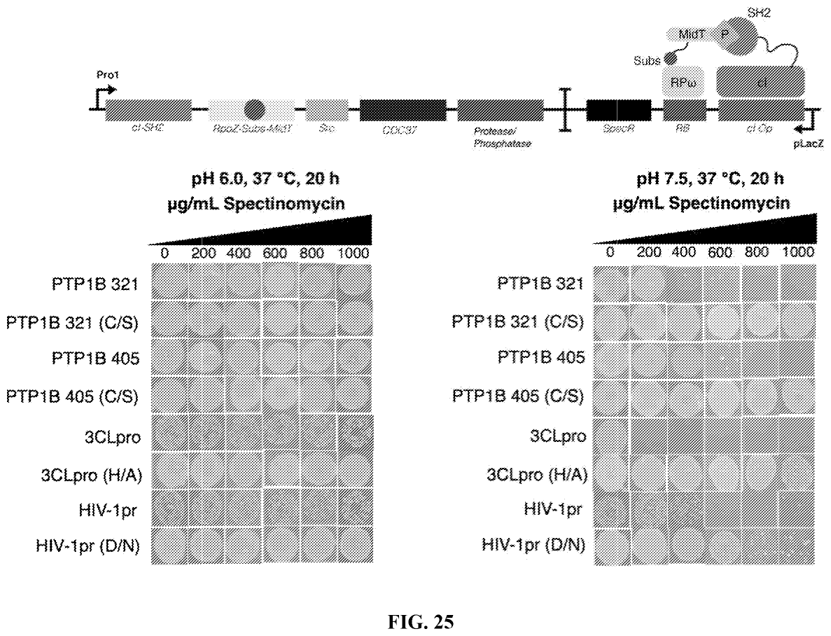

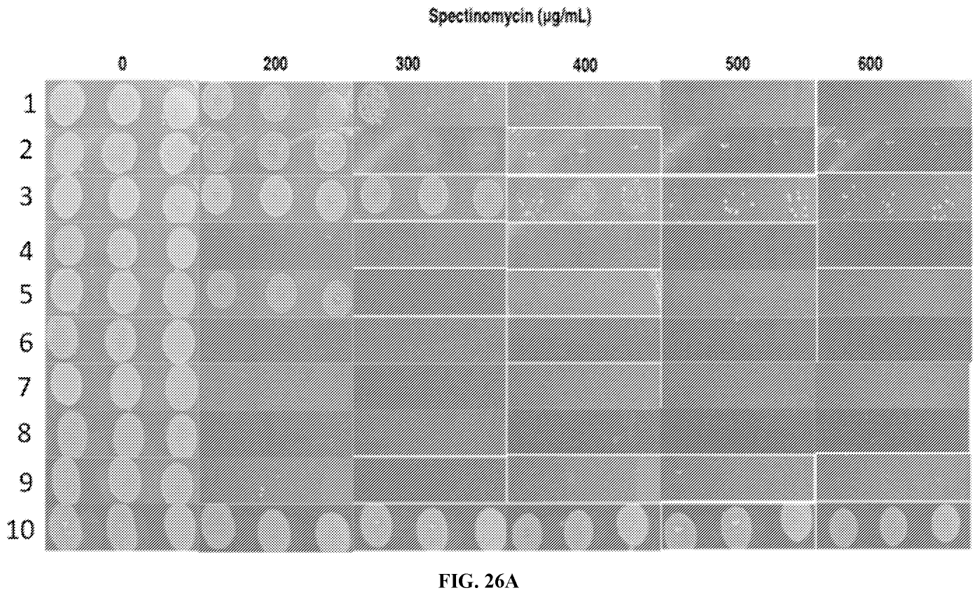

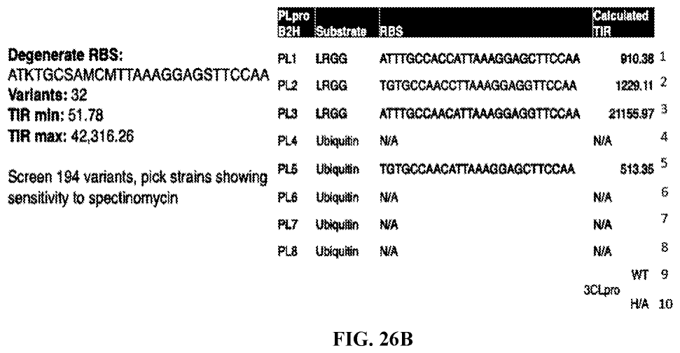

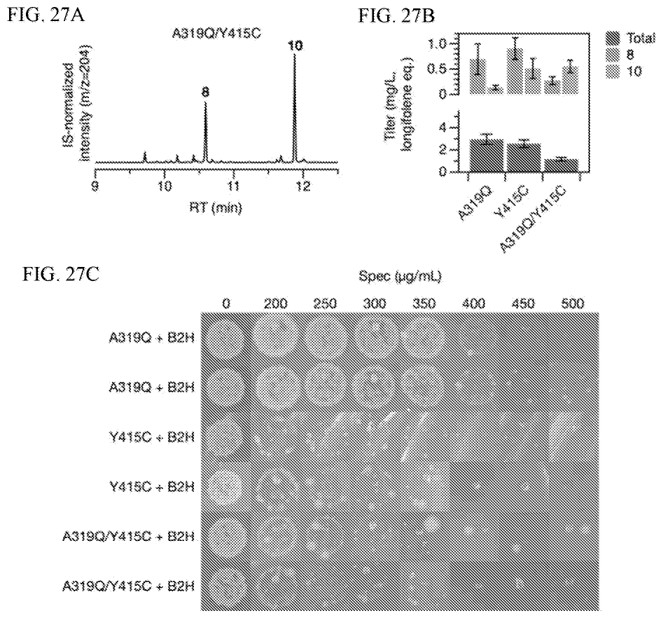

FIG. 18 shows a cladogram of terpene synthases screened for inhibitor production.

To search for biosynthetic pathways producing protease inhibitors, the B2H systems were paired with terpenoid pathways. These molecules and their derivatives have been shown to inhibit viral proteases and the construction of many diverse terpenes in E. coli can be achieved by exchanging just 1-2 genes in a biosynthetic pathway (a terpene synthase and/or prenyltransferase). To produce terpenes in E. coli, the B2H systems coupled the isopentenol utilization pathway (pIUP, which produces IPP and DMAPP from the cheap precursor, isoprenol) with an in-house terpene synthase library including 37 genes from a diverse set of organisms (FIG. 17, FIG. 18, and Table 7). To this pathway, the B2H systems added prenyltransferases producing two different sized terpene precursors: farnesyl pyrophosphate synthase (FPPS, producing C₁₅ precursors) and geranylgeranyl pyrophosphate synthase (GGPPS, producing C₂₀ precursors). When paired with a plasmid containing a terpene synthase gene (pTS), these pathways produced amorphadiene (C₁₅), and abietadiene (C₂₀) in titers ranging from 1.88-15.05 mg/L in lysates and 121.16-1463.01 mg/L intracellularly (measured as caryophyllene equivalents, FIG. 17). Inclusion of the prenyltransferase on pIUP minimized the number of plasmids required to test each terpene synthase with each precursor length (e.g. 74 different pathways). There is growing evidence that many terpene synthases are able to cyclize multiple precursors (e.g., a single terpene synthase can generate as many as 50 products); thus, it was hypothesized that testing each combination would maximize the molecular diversity of the screen.

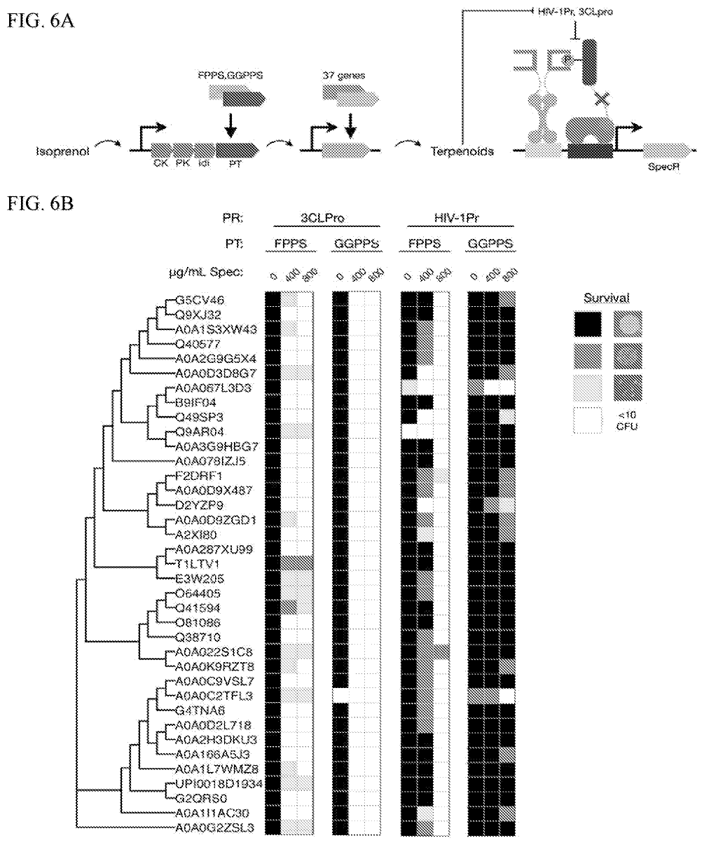

FIGS. 6A-6B show a strategy for combinatorial pathway screening. The terpenoid pathways were introduced on two plasmids containing (i) the IUP precursor pathway to convert isoprenol into FPP or GGPP and (ii) a terpene synthase pathway containing one of 37 genes from an in-house library. These pathway combinations were combined with the HIV1-Pr and 3ClPro B2H systems.

To search for terpenoid inhibitors of viral proteases, the pathways were paired with each protease responsive B2H. In the presence of a GGPP producing precursor pathway, pathways conferring survival against 3ClPro were not observed. Nearly all pathways, however, did provide a survival advantage with HIV-1Pr, suggesting (i) the GGPP precursor may be inhibiting this enzyme or (ii) the stringency of the HIV1-Pr selection against GGPP pathways needs to be increased (e.g. the active HIV1-Pr should die at lower concentrations of spectinomycin). In the presence of an FPP producing precursor pathway, several terpene synthases were observed conferring high levels of resistance (e.g. growth at 800 μg/mL spectinomycin) with one or both protease targets (FIGS. 6A-6B). Interestingly, 10 of the pathways showing robust survival did so for only one protease. These results suggest the production of terpenoids that specifically target either HIV1-Pr or 3ClPro. Furthermore, two canonical diterpene synthases (064405 and Q41594) and one canonical monoterpene synthase (UPI0018D1934E) were observed conferring resistance when paired with a sesquiterpene precursor. Truncations of Q41594, taxadiene synthase, and O64404, abietadiene synthase, have been shown to act on FPP in vitro to produce bisabolene-type sesquiterpenes or the acyclic molecule farnesene, respectively. 1,8-cineole synthase (UPI0018D1934E) has not been reported to act on FPP. These results justify further investigation of these pathways and indicate that the system can efficiently identify molecules with interesting properties being produced by unexpected enzyme combinations.

Nature excels in the development and production of functional molecules. Over millennia, random mutations and recombination events have produced myriad enzymatic pathways which, challenged by natural selection, yield useful compounds. In this study, the expression systems disclosed herein were engineered to apply artificial selection pressures to recapitulate this process in engineered E. coli. Using a bacterial two-hybrid architecture, the procedure developed and/or characterized systems for detecting inhibitors of HIV1-Pr and 3ClPro, proteases necessary for the infectiousness of two epidemic-causing viruses, by inserting cleavage sites into the B2H's flexible linkers. Using this strategy allowed for creation of systems producing either luminescence or spectinomycin resistance as output signals. The luminescent output allowed for quantification of system performance and identify optimal linker constructs, streamlining the development of the final antibiotic resistance-based system. This optimization revealed that the residues flanking the inserted cleavage site affect system performance depending on the protease used. It also helped identify a putative protease recognition site within RpoZ. Fortunately, a functional B2H system was still able to be developed, but non-targeted proteolysis of different B2H components (e.g. Src, CDC37, or the luciferase/spectinomycin resistance proteins) could complicate other designs.

To demonstrate the utility of the B2H, 74 terpene synthase pathways were screened against the HIV1-Pr and 3ClPro systems. Using a drop-plating assay, several terpene synthase pathways were identified conferring resistance to spectinomycin (implying protease inhibition) in the presence of one or both proteases. These pathways and their products merit follow up, both for evaluation of biosynthesized inhibitors (some of which may exhibit target selectivity) and investigations into the functionality of unexpected prenyltransferase/terpene synthase combinations (e.g., FPPS and 1,8-cineole synthase). Larger screens would benefit from improvements in assay throughput, such as barcoding strategies that allow pooling of pathways and measurements of fitness differences with next generation sequencing. Similar approaches have streamlined genome-wide studies of fitness-enhancing or reducing alterations, suggesting comparable improvements in the throughput of fitness measurements using plasmid-borne systems-like the terpenoid pathways and B2H systems—could be possible.

Bacterial two-hybrid systems were developed that detects the activity of two important disease-relevant proteases in E. coli, and the B2H systems used them to screen 74 terpenoid pathways for potential inhibitors. Several pathways were identified that improve resistance in the presence of HIV1-Pr, 3ClPro, or both—an indication of inhibitor biosynthesis. The findings described herein show that the B2H architecture can be adapted to other classes of drug targets. When paired with existing biosynthetic pathways for building diverse compounds in E. coli, these B2H systems could accelerate the development of drugs against challenging targets. Chemically competent NEB Turbo cells was used to carry out cloning and E. coli s1030 for all B2H analyses.

Methyl abietate was purchased from Santa Cruz Biotechnology. Tris(2-carboxyethyl) phosphine (TCEP), bovine serum albumin (BSA), M9 minimal salts, phenylmethylsulfonyl fluoride (PMSF), and DMSO (dimethyl sulfoxide) were purchased from Millipore Sigma; glycerol from VWR; cloning reagents from New England Biolabs; and all other reagents (e.g., antibiotics and media components) from Thermo Fisher.

All plasmids were constructed using Gibson assembly. Table 6 describes the source of each gene; Table 7 describe the composition of all final plasmids. In all cases, LB indicates the LB Miller recipe. Agar concentration was 2% for all solid media. When necessary, chemically competent cells were generated for cloning with the standard RbCl protocol, and electrocompetent cells with a washing protocol as previously described. For screening terpene synthases against proteases, the chemically competent cells were generated as follows: (i) From a glycerol stock, the s1030 cells harboring the pIUP and pB2H variant of interest were streaked and grew them on LB agar with plasmid antibiotics (kanamycin, tetracycline, chloramphenicol, concentrations listed in Table 7) at 37° C. (ii) The following day, the a single colony was picked to inoculate 2 mL LB with the same antibiotics and grew the culture at 37° C. and 225 RPM for 16 hours. (iii) A 1:100 dilution in 50 mL LB was created with the same antibiotics and grew the culture at 37° C. and 225 RPM until the OD₆₀₀ reached 0.3-0.6. (iv) The cells were pelleted at 5,000 RPM for 5 minutes. (v) The cells were resuspended in 500 μL ice cold 100 mM CaCl₂)+7% (v/v) DMSO and froze 100 μL aliquots at −80° C.

Preliminary B2H systems (which contained LuxAB as the GOI) were characterized with luminescence assays. Plasmids were transformed into s1030, plated the transformed cells onto LB agar plates+plasmid antibiotics (Table 7), and incubated all plates overnight at 37° C. The following day, colonies were picked to inoculate 1 mL LB cultures with the same antibiotics and grew the culture at 37° C. and 225 RPM for 16 hours. The following morning, each culture was diluted by 100-fold into 1 ml of TB media and incubated these cultures in individual wells of a deep 96-well plate for 5.5 hours (37° C., 225 RPM), including arabinose when a pBAD plasmid was present. 100 μL of each culture was transferred into a single well of a standard 96-well clear plate and measured both OD₆₀₀ and luminescence on a Spectramax iD3 plate reader (standard luminescence settings). Cell-free media was measured and subtracted the signals from each measurement prior to calculating OD-normalized luminescence (e.g., Lum/OD₆₀₀).

Drop-plating of E. coli cells lacking a metabolic pathway was carried out as follows: (i) The B2H plasmid was transformed into s1030 cells (electroporation) and plated on LB agar+plasmid maintenance antibiotics (kanamycin and tetracycline, antibiotic concentrations listed in Table 7) and grown overnight at 37° C. (ii) Colonies were picked to inoculate 1 mL TB (12 g/L tryptone, 24 g/L yeast extract, 12 mL/L 100% glycerol, 2.28 g/L KH₂PO₄, 12.53 g/L K₂HPO₄), pH=7.0+plasmid maintenance antibiotics and shaken overnight (225 RPM, 37° C.). (iii) The following morning, each culture was diluted to OD₆₀₀=0.1 in 1 mL TB, pH=7.0 (no antibiotics), 5-7 μL of each dilution was drop-plated onto LB agar (pH=7.5+plasmid maintenance antibiotics and increasing concentrations of spectinomycin), and the plates were grown at 37° C. (iv) The following morning, plates were photographed.

Drop-plating of E. coli cells producing terpenoids was carried out as follows: (i) pB2H, pIUP, and pTS were transformed into s1030 cells (electroporation) and plated on LB agar containing kanamycin, tetracycline, chloramphenicol, and carbenicillin. (ii) Colonies were picked to inoculate 2 mL TB, pH=7.0+plasmid maintenance antibiotics and shaken overnight (225 RPM, 37° C.). (iii) The following morning, each culture was diluted as before and each dilution was drop-plated on LB agar (pH=7.0)+2% glycerol, 10 mM isoprenol, 50 μM iPTG, plasmid maintenance antibiotics, and increasing amounts of spectinomycin, and the plates were grown at 22° C. (v) plates were photographed after 72 hours.

Terpenes were produced by transforming all necessary plasmids into E. coli cells (see Table 7 for strain/plasmid details) and plating on LB agar plates containing antibiotics for plasmid maintenance. Following overnight growth at 37° C., colonies were picked to inoculate 2 mL TB+antibiotics and grown overnight in an incubator shaker (37° C., 225 RPM). The following morning, cultures were diluted 1:75 into TB+antibiotics and grown (37° C., 225 RPM) until the OD₆₀₀=0.3-0.6. Once reaching the required OD₆₀₀, cultures were induced by adding isoprenol (50 mM) and iPTG (50 μM or 500 μL) and then transferred to an incubator shaker at 22° C., 225 RPM for 48 hours.

Terpenoids were produced in vivo in 4 mL cultures as described above. At the completion of each fermentation, the OD₆₀₀ was measured and the total cellular volume in 1 mL of the culture was determined from the specific cellular volume for complex media containing glycerol and amino acids (assumed to be similar to TB). Lysate terpenoids (cells+media) were extracted by: (1) adding 1 mL culture to 600 μL hexane (2) vortexing hexane/cell mixture for 3 minutes (3) centrifuging the mixture at 17,000×g for 2 minutes (4) retaining 400 μL of the resulting hexane layer and storing at −20° C. for further analysis. Intracellular terpenoids were extracted by: (1) removing an additional 1 mL culture and centrifuging at 4,000×g for 3 minutes (2) discarding the supernatant and adding 100 μL disruptor beads (Chemglass, CLS-1835-BG1)+600 μL hexane and (3) vortexing the bead/hexane mixture for 3 minute. Samples were centrifuged and stored as before.

Terpene titers and compound identity were analyzed by using a Trace 1310 GC fitted with a TG5-SilMS column and an ISQ 7000 MS with the following GC method: hold at 80° C. (3 min), increase to 250° C. (15° C./min), hold at 250° C. (6 min), increase to 280° C. (30° C./min), and hold at 280° C. (3 min). For compound identification, the m/z ratios from 50-550 were scanned and assigned ID's (when possible) using comparisons to compounds in the NIST MS library. For compound quantification, the single ions (m/z=121 for sesquiterpenes, and m/z=93 for diterpenes) were scanned. Samples included an internal standard, caryophyllene, at a constant 20 μg/mL. Injections where the internal standard area was greater than ±50% different from the average of all samples from a given day were repeated. Terpene titers for compounds i were determined as caryophyllene equivalents using equation 4-1, where sid=caryophyllene:

$${C}_{i} = {C}_{std} * \frac{{A}_{t}}{{A}_{std}} \qquad Eq . V - 4$$

Multiple sequence alignments were created for all cladograms using the Muscle algorithm in MegaX. Following alignment, the maximum-likelihood tree using MegaX was created with default settings. Tree visualization was carried out in R studio using the ggtree package.

To align 3ClPro from SARSCOV and SARsCoV2, EMBOSS Needle was used.

In some embodiments, a protease recognition sequence

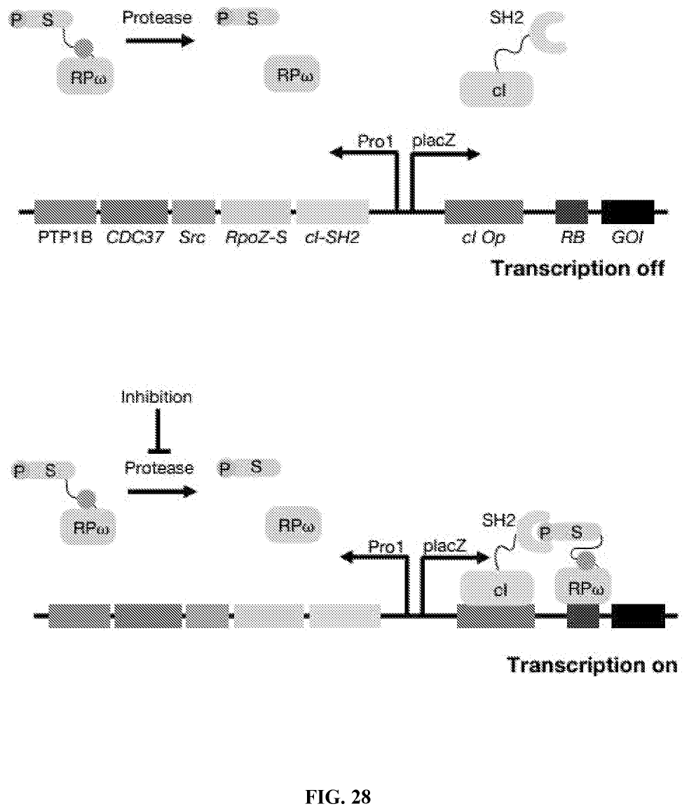

FIG. 28 shows an embodiment of a bacterial two-hybrid system that detects protease inhibitors. The components include (i) a substrate domain fused to the omega subunit of RNA polymerase or portions thereof, (ii) an SH2 domain fused to the phage cI repressor, (iii) an operator for cI, (iv) a binding site for RNA polymerase, (v) SRC kinase, (vi) a protease cleavage site, and (vii) a protease. SRC-catalyzed phosphorylation of the substrate domain enables a substrate-SH2 interaction that activates transcription of a gene of interest. Protease-catalyzed hydrolysis of the protease cleavage site prevents the transcription of the gene of interest. A protease inhibitor would reduce the protease-catalyzed hydrolysis and increase the transcription of the gene of interest.

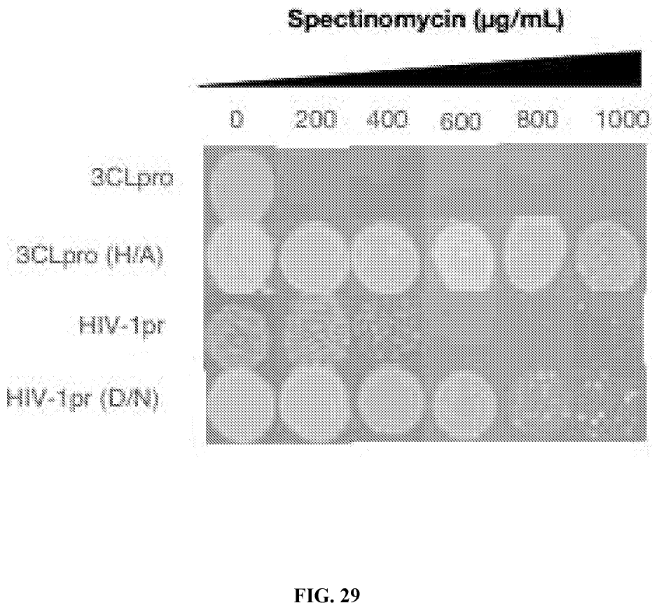

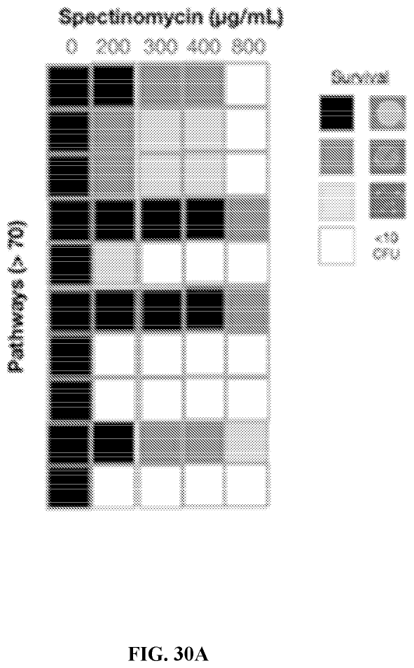

FIG. 29 shows an analysis of functional bacterial two-hybrid systems that detect inhibitors of 3CLpro or HIV-1pr and that contain a gene for antibiotic resistance as the gene of interest (GOI). The analysis shows that bacterial systems with a functional protease (first and third rows with 3CLpro and HIV-1pr, respectively) do not confer survival at high concentrations of antibiotic. No cells survive above 0-400 μg/mL of the antibiotic spectinomycin. However, if the protease activity is reduced, in this case by mutation (second and fourth rows with 3CLpro H/A and HIV-1pr D/N, respectively), transcription of the antibiotic resistance GOI is increased, and survival is conferred at higher concentrations of spectinomycin.

FIGS. 30A-30C shows the results of a screen for inhibitors of 3CLpro. FIG. 30A shows that several pathways confer survival in the presence of a bacterial two-hybrid system that detects inhibitors of 3CLpro. FIG. 30B shows the inhibition of 3CLpro-mediated cleavage of a FRET peptide by different levels of a mixture containing α-bisabolol. FIG. 30C shows a dose-response curve for the inhibition of 3CLpro by a mixture containing α-bisabolol.

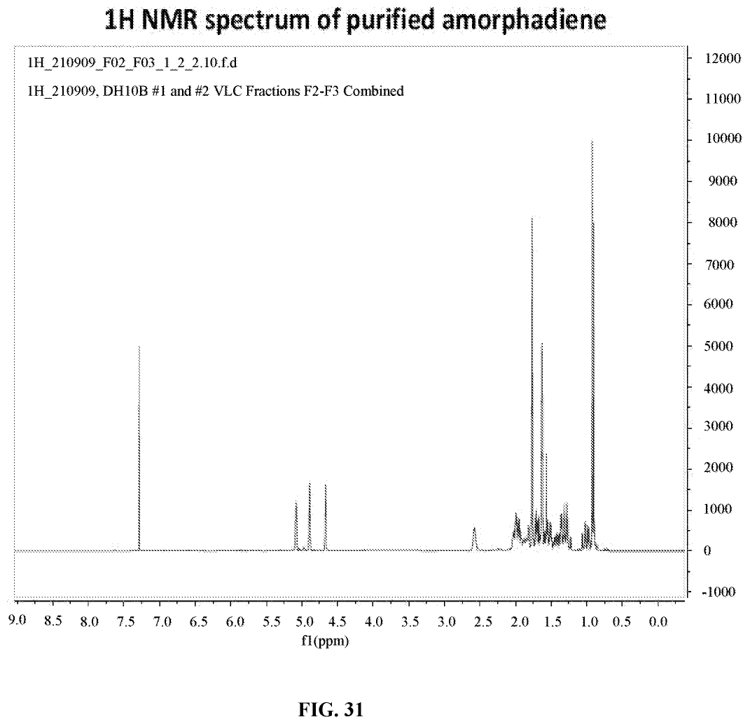

FIG. 31 shows a 1H NMR spectrum of purified amorphadiene. The amorphadiene was produced from a lab-scale fermentation and purified with vacuum liquid chromatography, yielding >200 mg/L amorphadiene. The large yield, simple purification, good purity, and clear optimization path (FIGS. 34A-34B) demonstrate that amorphadiene is a good starting compound for optimization of a phosphatase inhibitor.

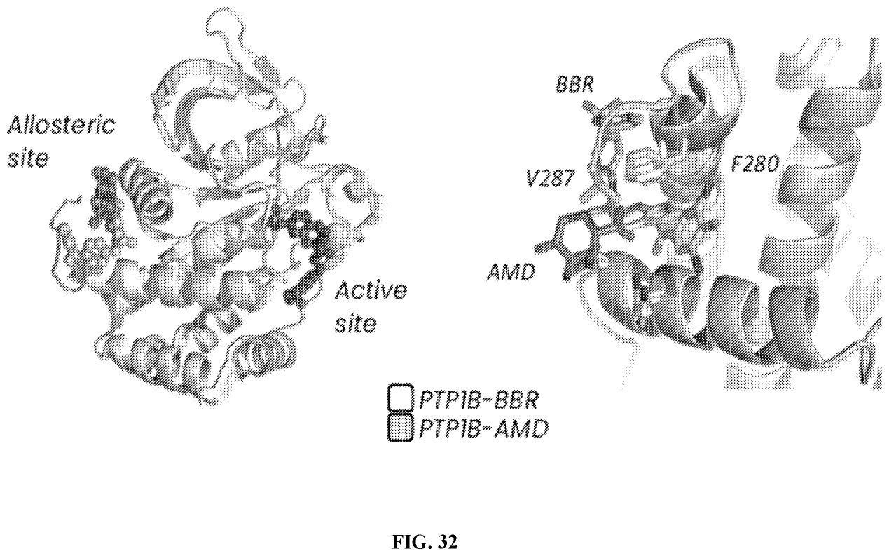

FIG. 32 shows aligned crystal structures where amorphadiene and BBR bind to the same allosteric site but do not overlap. The crystal structures include both an active site and an allosteric site. Both BBR and amorphadiene bind at the allosteric site.

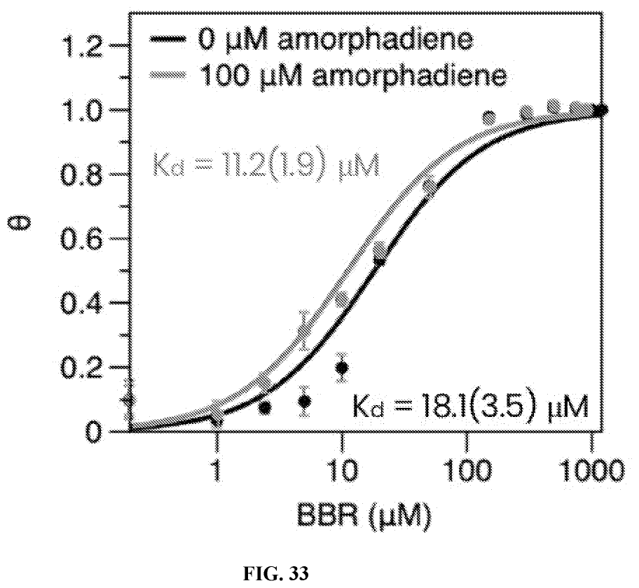

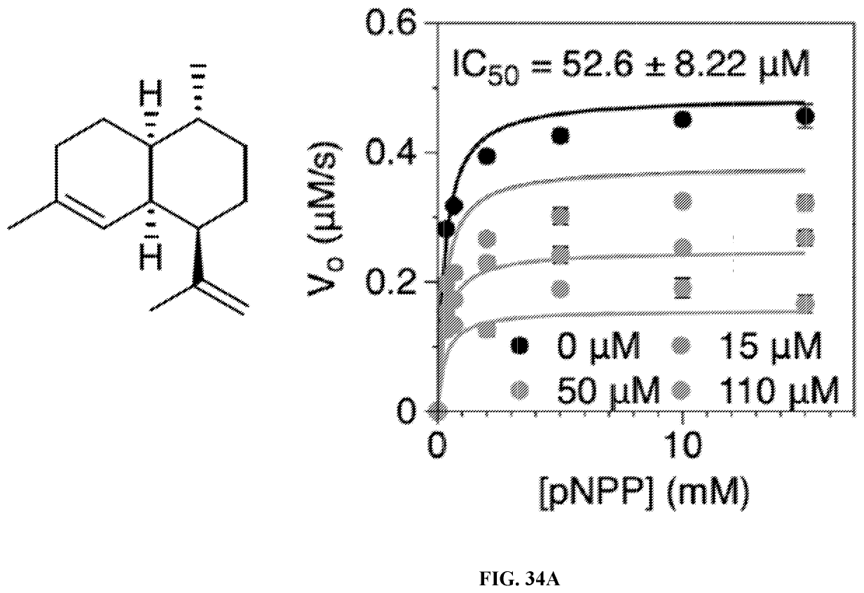

FIG. 33 shows that binding of BBR to PTP1B is not significantly disrupted by the presence of 100 M amorphadiene.

FIG. 34A-34B shows the inhibition of PTP1B by amorphadiene (FIG. 34A) and by a propargyl derivative of amorphadiene (FIG. 34B). The propargyl derivative is more water soluble than amorphadiene, and is thus more ‘drug-like’, while the two compounds have similar inhibitory potencies. Based on the crystal structure of amrophadiene bound to PTP1B, the propargyl functional group likely projects into the allosteric binding site. The addition of the propargyl group demonstrates that amorphadiene can be modified with functional groups directed into the binding site with no loss in potency. Further, the propargyl group is an ideal group for further modifications (e.g., with BBR-like functional groups) to improve the potency of the amorphadiene starting compound.

### Example 3: Expanded Screens

An approach for using genetically encoded systems was developed to guide the discovery of targeted, biologically active molecules in microbial hosts. The work began with the development of a bacterial two-hybrid (B2H) system that links the activity of protein tyrosine phosphatase 1B (PTP1B), an elusive drug target, to the expression of an antibiotic resistance gene to in E. coli. This system was used to screen 29 terpenoid pathways and identified two inhibitors with surprising potency and binding modes. Building on these results, the same system was used to evolve a terpene synthase to confer a survival advantage in the presence of the PTP1B-focused B2H system; in this effort, the B2H system identified mutants that increase the production of total terpenes in E. coli and/or shift its product profile to enhance the titers of minor components. This study also revealed a previously unreported residue important for directing 6,11 ring closure during catalysis; removal of the hydroxyl functionality at this site yielded significant shifts in product profile toward bicyclic molecules. This work concluded with the development of two-hybrid systems that detect the activity of viral proteases; using these systems, a combinatorial screen of 74 biosynthetic pathways was carried out, identifying enzyme combinations that modulate the activity of each protease system in distinct ways. These findings demonstrate the compatibility of the two-hybrid architecture with other classes of diseases-relevant enzymes.

The work with a PTP1B-specific bacterial two-hybrid motivates screens of other PTP-based systems. Combining the two-hybrid system harboring PTP1B with terpenoid pathways led to the discovery of molecules with surprising degrees of specificity against other phosphatases—a property that has eluded past drug development efforts. Motivated by these results, the approach showed that the two-hybrid system could incorporate other PTPs of medicinal relevance without further optimization and that the responses of these systems are consistent with the selectivity of biosynthesized inhibitors (e.g., the pathway for amorphadiene, which is a more potent inhibitor of PTP1B than TCPTP, conferred a better survival advantage alongside the PTP1B-specific B2H system than it did for the TCPTP-specific system). The approach envisioned using alternative PTP-specific B2H systems not only for identifying inhibitors of alternative PTPs, but also for carrying out high-throughput screens that enable the identification of metabolic pathways for selective inhibitors. A screen of biosynthetic libraries against multiple PTP-specific B2H systems, for example, should enable the identification of pathways that produce selective inhibitors.

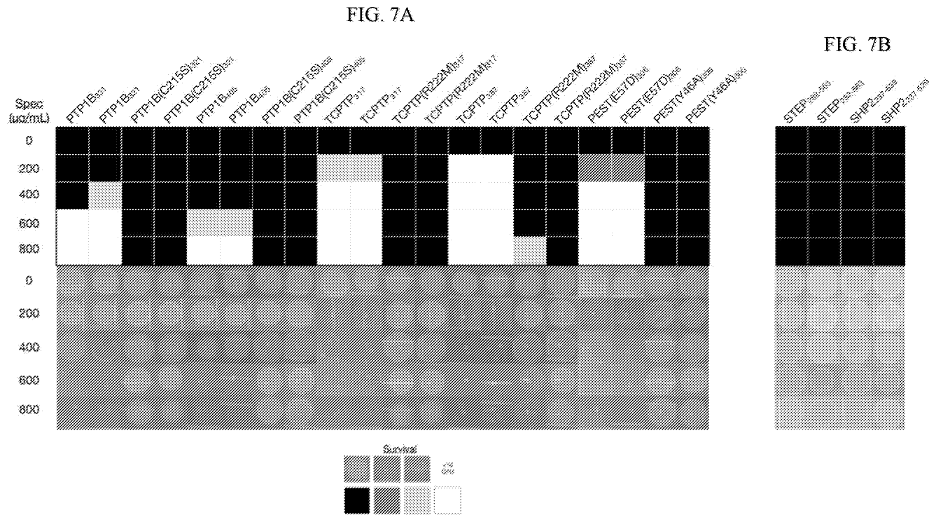

FIGS. 7A-7B show functional systems (defined as reduced growth with an active PTP vs. an inactive PTP). Subscripts indicate the truncation used for each enzyme. PTP1B₄₀₅ and TCPTP₃₈₇ include C-terminal regions beyond the conserved catalytic PTP domain. All mutations in parentheses are inactivating except for PEST (E57D), which is a cancer-associated variant.

Long isoforms of PTP1B and TCPTP (harboring disordered and/or hydrophobic domains) are also compatible with the B2H design (FIGS. 7A-7B). These full-length (or near full-length) enzymes may contain poorly conserved allosteric sites that enable selective inhibition; importantly, full-length PTPs are difficult to evaluate with classical in vitro screens because their disordered regions tend to misfold or aggregate in solution. The simultaneous use of multiple B2H systems with different PTP isoforms to screen large biosynthetic pathways could enable the identification of molecules that target poorly conserved domains—and that act through allosteric binding modes that are likely to be enzyme-specific. A similar in vitro screen against Src-homology region 2-containing region phosphatase-2 (SHP2) revealed an allosteric mode of inhibition and led to an explosion in inhibitor development and clinical studies.

Unfortunately, not all PTPs are easy to incorporate into the B2H design. For example, striatal-enriched phosphatase (STEP, a potential target for neurological diseases) and SHP2 (a validated cancer target) did not yield functional two-hybrid systems, most likely due to low activity against the MidT substrate (FIGS. 7A-7B). For these and other non-functional constructs, improvements in PTP expression or substrate compatibility (e.g., the use of artificial spacers or structured proteins that better mimic the native targets of phosphatases) could improve B2H performance. Notably, some PTPs exhibit large differences in activity on different substrate sequences; thus, alternative, PTP-specific substrates may be necessary to yield functional systems. Finding usable substrates is non-trivial, as they must be compatible with Src kinase and the SH2 domain. B2H systems, however, could help identify substrates with the required properties because compatible substrates yield a measurable signal (e.g. luminescence or growth).

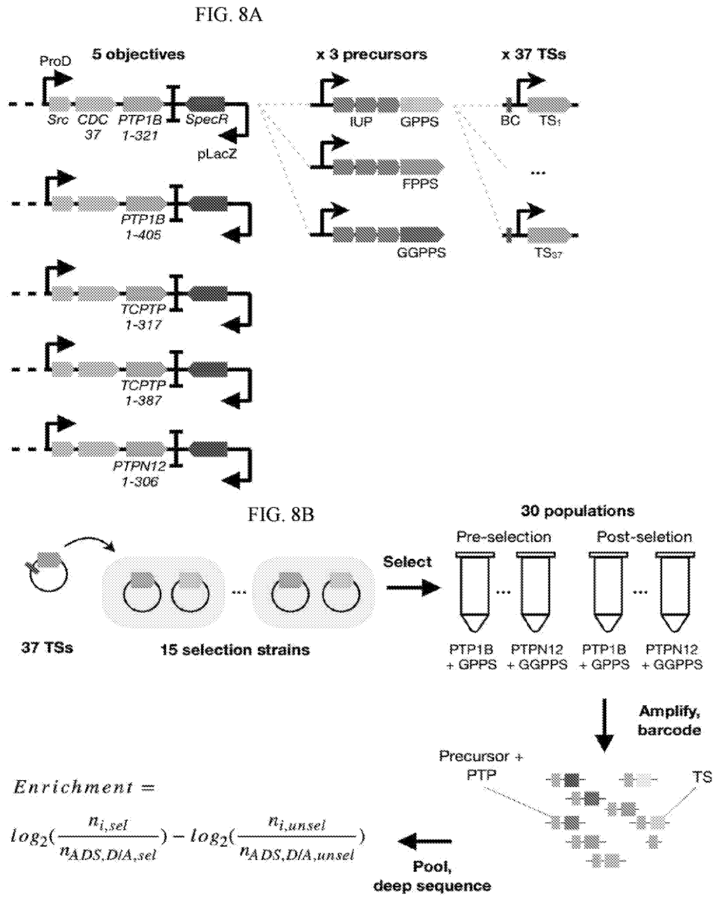

FIGS. 8A-8B show a hypothetical screen of five target PTPs (“objectives”) and 111 terpenoid pathways (3 precursors×37 terpene synthases with barcodes “bc”). Barcoded terpene synthases are pooled and transformed into selection strains harboring every possible precursor/objective combination. These transformations are plated on selective and non-selective media and each resulting population's DNA is recovered, amplified with barcodes specific to the precursor/objective/selection condition, pooled, and sequenced. The equation shown can be used to calculate enrichment of a given terpene synthase plasmid (n, barcode count in selected or unselected population, N_{ADS,D/A}=barcode count an inactive variant of a terpene synthase that does not significantly impact growth of E. coli, but also does not confer a significant survival advantage in the system.

The extension of the B2H screens to large numbers of pathways and protein targets will require enhanced throughput. Pooling of barcoded biosynthetic pathways followed by next generation sequencing measurements of barcode abundance could improve screening efficiency. In a pilot experiment, the approach combined (i) three isoprenoid pathways, (ii) 37 terpenoid pathways, and five PTP-specific B2H systems in a single screen. The 555 possible combinations of these three sets of plasmids would be challenging to screen with drop-based plating (FIG. 8). By barcoding both (i) the terpenoid pathways and (ii) the B2H systems, the required number of transformations could be reduced to 15 (e.g., one for each precursor-B2H combination); the consolidation of biosynthetic pathways on a single plasmid can reduce this number further. In the pilot experiment, each transformation was plated on both selective and non-selective media, the pools from each plate were amplified with a PCR reaction that introduces a second barcode for the PTP of interest, and next generation sequencing was used to measure the enrichment of specific pathways (FIG. 8). High quality, statistically significant data can usually be obtained for a maximum 10-10 variants when sequencing short amplicons, suggesting that the proposed strategy should be compatible with very large biosynthetic libraries and/or numerous target two-hybrid systems.

FIGS. 9A-9B show B2H design for T7-based amplification of a fluorescent protein (FP). Src kinase (not shown) phosphorylates the MidT substrate, enabling binding to an SH2 domain followed by RNAP localization and expression of SpecR+T7 RNAP. T7 RNAP drives expression of a fluorescent protein (FP) from a plasmid borne T7 promoter. PTP1B prevents MidT/SH2 binding and transcription by dephosphorylating the substrate. SpecR is included as a secondary selection marker if needed. Fluorescent signal where FP=superfolder GFP (sfGFP) or α-GFP (uvGFP). Error bars denote standard deviation for n=4 biological replicates. WT=wild-type, C215S-catalytically inactivating mutation.

In this study, the approach demonstrated that the detection systems are compatible with large mutagenesis libraries, in addition to large pathway libraries. The first growth-coupled assay used to carry out directed evolution of a terpene synthase was reported to improve its ability to generate a biologically active molecule. This assay allowed us to screen thousands of enzyme variants on selective media to identify variants with improved titers, shifted product profiles, and lowered burdens on cell growth. Although growth-based selections are valuable for their throughput, their reliance on survival can be confounded by other fitness effects, such as the toxicity or metabolic burden of heterologously expressing many genes. Screening mutagenesis libraries of a poorly tolerated terpene synthase in E. coli against the PTP1B-based two-hybrid system yielded strains with improved growth that was partially independent of B2H modulation. Using T7 RNAP as the gene expressed by the B2H, amplification systems were built that, following PTP1B inactivation, show large increases in fluorescent protein expression from a plasmid encoded T7 promoter (FIGS. 9A-9B). The approach envisioned using this system to screen or evolve enzymes with significant fitness impacts on E. coli, such as terpenoid pathways harboring cytochrome P450s (which enable oxygenation) from plants.

The approach modified the two-hybrid architecture to accommodate viral proteases. The resulting systems demonstrate how the original detection system can be extended to other drug targets. Although the targets were screened with the terpene synthase library, the structures of some previously reported protease inhibitors resemble those of other natural products (e.g., flavonoids or non-ribosomal peptides); incorporating pathways responsible for their production may also yield compounds with pharmaceutically relevant properties. To take better advantage of more natural product classes, hosts other than E. coli may be important. Organisms like those of the Streptomyces genus are capable of producing more complex molecules, and genome minimized versions of certain species are available for heterologous biosynthesis with minimal background natural product production. Intriguingly, the RNA polymerase structure of Streptomyces coelicolor (previously engineered to produce non-native molecules), could be compatible with a bacterial two-hybrid system similar to the one developed in this thesis. Specifically, the α-subunit (rpoA) shares ˜60% sequence identity with E. coli's rpoA and is functional in both organisms. In E. coli, RpoA can play a similar role as rpoZ in the bacterial two-hybrid system without any genomic modification (rpoZ requires a scarless deletion); Initial systems will likely focus on detecting proteases or peptidases—several of these enzymes are known to express in the Streptomyces genus. The resulting systems can then be screened with biosynthetic pathways producing a wide range of natural product classes.

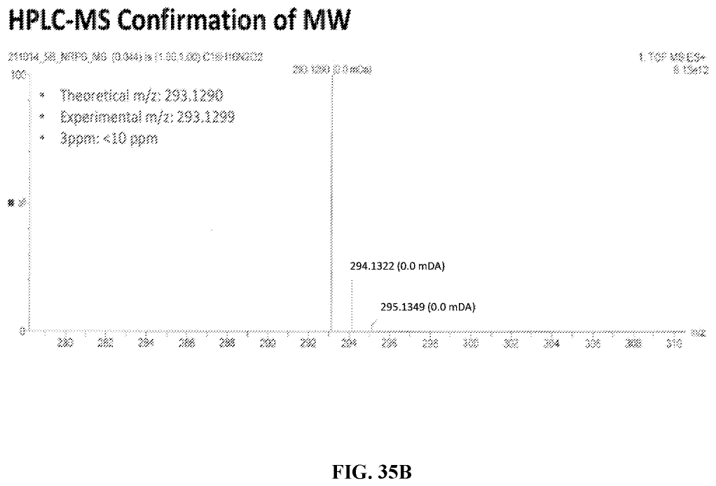

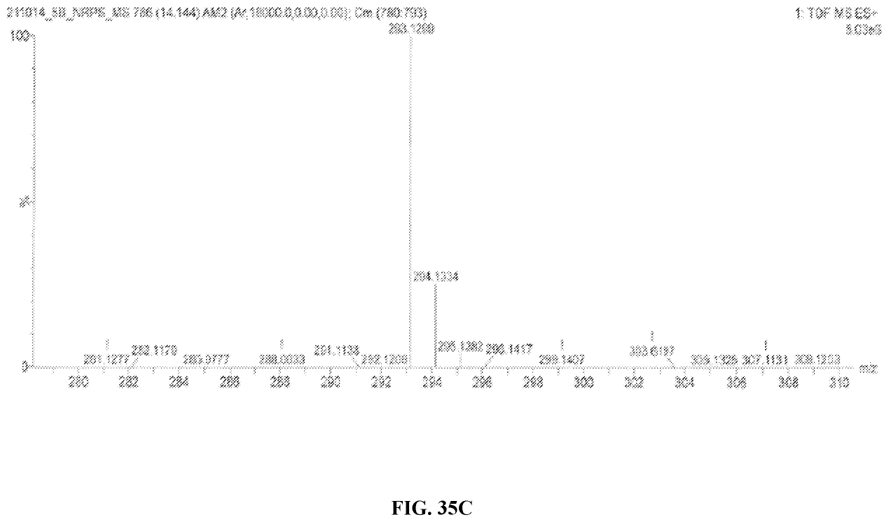

FIG. 35A-35C shows the implementation of biosynthetic pathways that produce non-ribosomal peptides and the confirmation of the production of a particular non-ribosomal peptide. A fermentation extract was analyzed by HPLC-UV and HPLC-MS to confirm the presence of a dipeptide pyrazine.

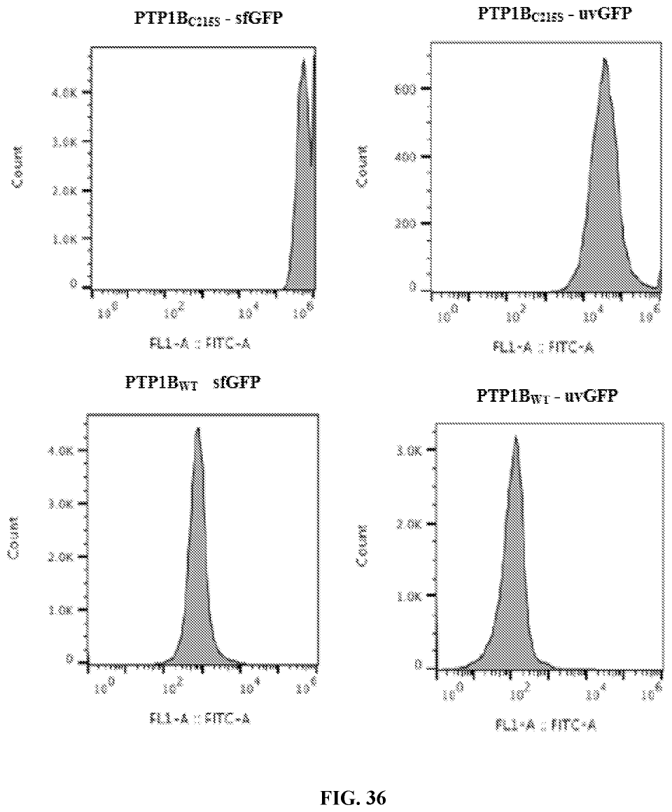

FIG. 36 shows the analysis of fluorogenic B2H systems by fluorescence-activated cell sorting. These fluorogenic B2H systems have a T7 RNA polymerase as a gene of interest and are accompanied by a gene for GFP under control of a T7 promoter. In this embodiment, one plasmid harbors the B2H system, and a second plasmid harbors the gene for GFP under control of a T7 promoter. Fluorescence-based screens can be advantageous compared to survival- or growth-coupled screens in some instances. Two fluorogenic B2H systems were constructed with different variants of green fluorescent protein (GFP). In each case, the B2H system with active PTP1B has a reduced fluorescence (e.g., a reduced transcription of the fluorescent protein GOI) compared to the same system with an inactive PTP1B. Therefore, these fluorogenic B2H systems are capable of detecting PTP1B inhibitors, and FACS can be used to sort and recover individual microbial cells harboring biosynthetic pathways conferring different levels of PTP1B inhibition.

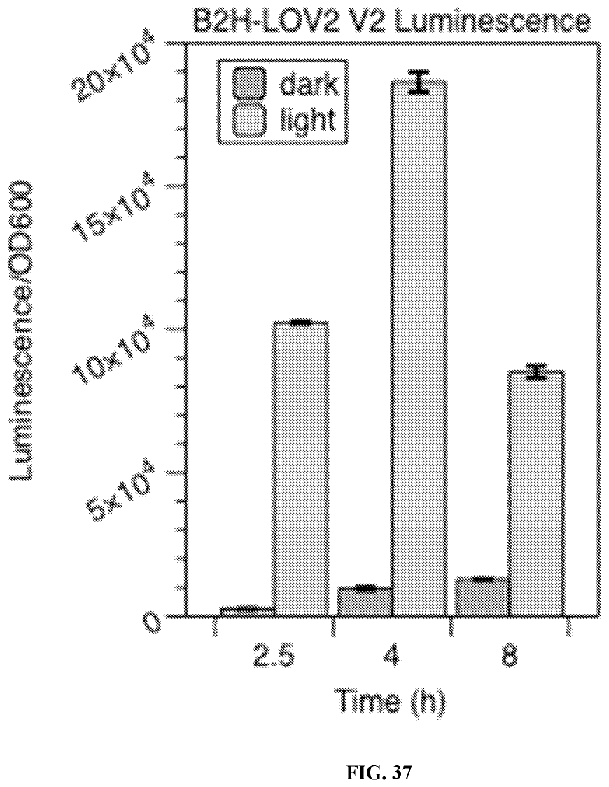

FIG. 37 shows the use of optical switches to enable precise control over the on/off state of a luminescent B2H system. A bacterial two-hybrid system was constructed with (i) a variant of a light-oxygen-voltage 2 (LOV2) domain that contains a bacterial SsrA peptide and (ii) a modified SspB peptide in place of the substrate and SH2 domains that are contained in other B2H designs. Exposure of LOV2 to light causes a conformational change that exposes the SsrA peptide and enables an SsrA-SspB interaction that promotes transcription of a gene of interest (GOI). The GOI is LuxAB in this embodiment. This type of photo-switchable system is valuable to control the dynamics of the B2H system to improve the production and/or detection of inhibitors.

### Example 4: B2H System with a Protease Recognition Sequence in the Linker

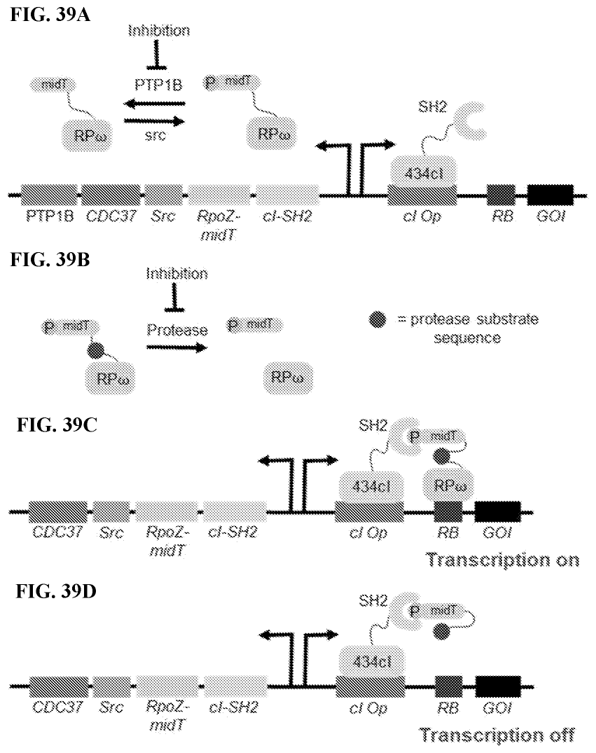

This example describes a B2H system that includes a protease recognition sequence in a linker that connects MidT to RpoZ (FIG. 39B). With this addition, an active protease prevents transcriptional activation by “breaking” the MidT-RpoZ fusion; inactivation of the protease reenables transcription (FIGS. 39B-39D). The design uses two additions to the PTP1B-based B2H system: (i) a protease-specific cleavage site between RpoZ and midT and (ii) the protease itself.

This works begins with a B2H system that links the inactivation of PTP1B to the expression of a gene of interest (GOI). In this system, Src kinase phosphorylates a substrate domain, causing it to bind to a Src homology 2 (SH2) domain, and the substrate-SH2 complex activates transcription of the GOI. PTP1B dephosphorylates the substrate domain, preventing transcription; the inactivation of PTP1B reenables it. Protease-specific detection systems do not require phosphorylation, but it was speculated that the substrate-SH2 interaction could be modified to detect proteases through the addition of protease-specific cleavage sites.

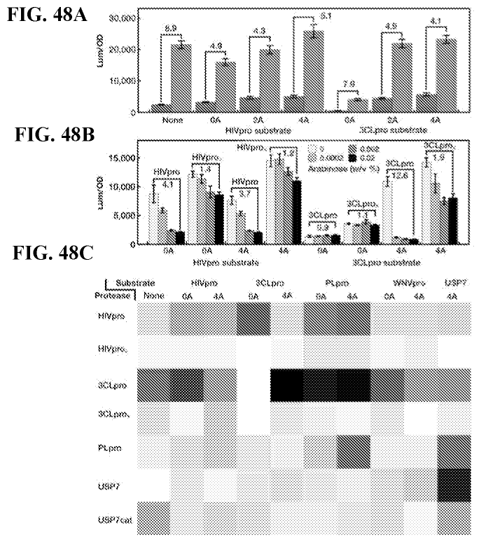

It was determined how protease-specific cleavage sites affect B2H function. In brief, recognition sequences for 3CLpro and HIVpro were added to the linker that connects the substrate domain to RpoZ (the omega subunit of RNA polymerase); these sites with 0-4 alanine residues (which were speculated to modulate protease access); and the output afforded by active and inactive PTP1B were measured, as shown in FIG. 48A. Cleavage sites reduced dynamic range up to two-fold but retained the desired B2H activity (e.g., PTP1B inactivation increased luminescence) at a 4- to 5-fold dynamic range. Cleavage sites flanked by four alanine residues tended to afford the highest luminescent signal.

Next, the sensitivity of the luminescent systems to protease overexpression was assessed. Here, we used B2H systems modified to contain both (i) protease-specific cleavage sites flanked by 0- or 4-alanine segments and (ii) an inactive PTP1B. In brief, we transformed E. coli with two plasmid-borne modules—(i) a B2H system and (ii) a protease induction system (an arabinose-inducible protease)—and we monitored changes in luminescence caused by protease expression (e.g., arabinose titration; FIGS. 48A-48C). Both HIV systems (e.g., 0- and 4-alanine variants) and one 3CLpro system exhibited a decrease in luminescence in response to protease expression. Importantly, the largest response was observed for the active protease. Inactive proteases caused a small decrease in luminescence, an effect that may result from weak substrate binding and/or a general cellular stress response to protease overexpression. The small response afforded by inactive proteases could reflect its mild affinity for substrate sequences, or, alternatively, a cellular response to protein overexpression.

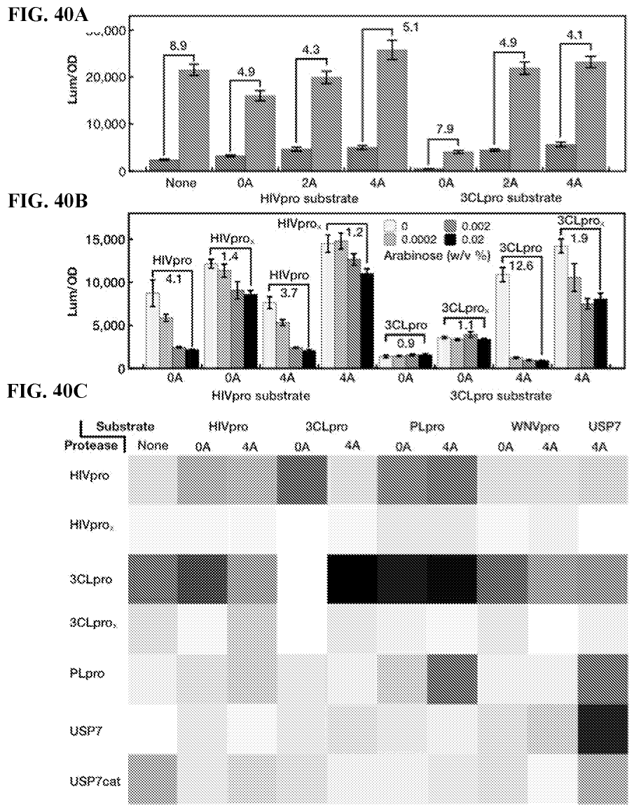

Additional proteases and protease-specific cleavage sites were screened. In short, recognition sites were added for the papain-like protease of SARS-COV-2 (PLpro), the NS2B/NS3 proteases of West Nile and Dengue Viruses (WNVpro and DVpro, respectively), and ubiquitin-specific protease 7 (USP7). These B2H systems were screened alongside the associated proteases (FIG. 40C). For USP7, the catalytic domain was included both with and without a C-terminal extension required for activity (to serve as positive and negative controls, respectively). Intriguingly, 3CLpro reduced luminescence for multiple recognition sites—which is an indication that one or more components of the underlying B2H system contains a cleavage site for 3CLpro (a site in RpoZ was in confirmed). PLpro and the extended version of USP7 were most active on their native recognition sequence; PLpro also showed activity on the ubiquitin-encoding sequence. This protease targets ubiquitin-like interferon-stimulated gene 15 protein. WNVpro and DNVpro showed no activity in initial screens.

Table 10 provides a non-limited list of viral proteases. In this example, 30 viral proteases were considered on the basis that associated viruses contribute to viral diseases with significant unmet medical need, high epidemic potential, and/or relevance to US biodefense. These diseases are listed as (i) priority pathogens by the National Institute of Allergy and Infectious Diseases (NIAID) 19 and/or (ii) priority emerging infectious diseases by the World Health Organization (WHO) 20. The disclosed of viral proteases of Table 10 includes 25 enzymes; each selected protein (or a close homologue) has at least one crystal structure and has been expressed in an active form in E. coli.

These proteases are considered for several reasons: (i) they may complement the modularity of the systems and methods disclosed (e.g., the platforms and/or workflows) for integrating new targets into the B2H system; (ii) data generated in screens may be used to prioritize hits based on unmet medical need, commercial opportunity, and molecular progressivity (e.g., ‘drug-likeness’ or synthetic tractability); and (iii) studying these proteases may inform about inhibitor specificity that could be used to further inform the design of broad-spectrum antivirals or shift focus away from non-selective inhibitors with potential toxicity issues.

### Example 5: B2H System Linking Target Inactivation to Cell Survival

To build survival-modulating systems for 3CLpro and HIVpro, two changes were made to a B2H system that includes a gene for spectinomycin resistance as the GOI: (i) a protease was swapped for PTP1B, and (ii) the best-performing cleavage site from our luminescence-based screen was added. To optimize these systems, ribosome binding sites (RBSs) were screened with different translation initiation rates (TIRs) and selected RBSs that enhanced sensitivity to spectinomycin.

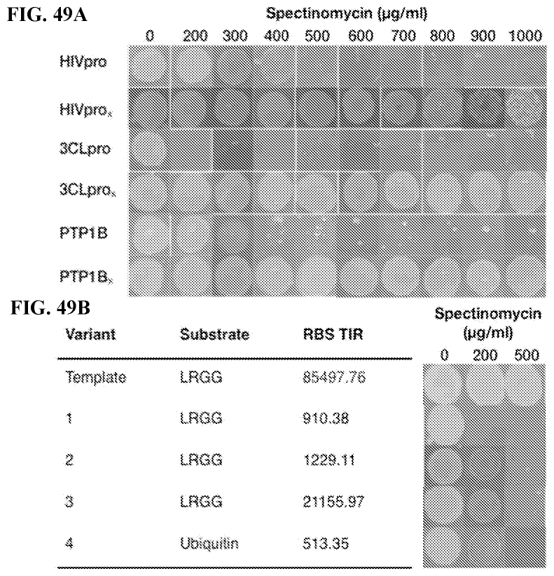

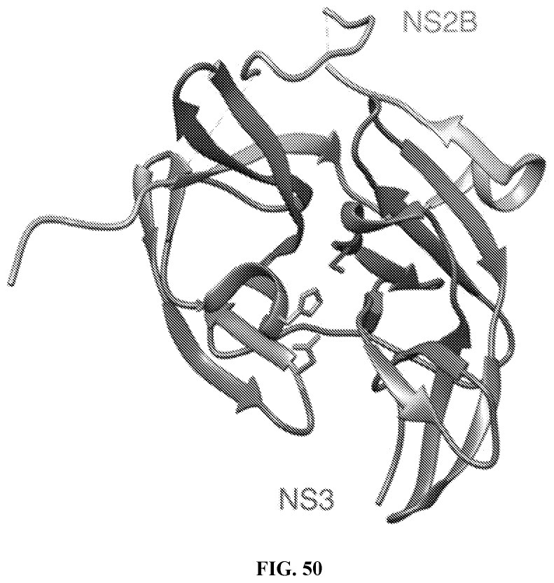

The analysis of luminescence-based systems suggests that protein expression is an important adjustable parameter for B2H development. To sample different expression levels without adding an inducer, the ribosome binding site (RBS) calculator, developed by the Salis Lab 174, was used to design RBSs with different translation initiation rates (TIRs), and these sites were screened with drop-based plating. This screen allowed the identification of RBSs for 3CLpro and HIVpro that link protease inactivation to an increase in spectinomycin resistance (FIG. 49A).

B2H development was continued by focusing on PLpro. To reduce the cloning required to sample different RBSs, degenerate primers were used to screen a small (˜200 member) library of TIRs spanning several orders of magnitude (50-100,000). This rapid screen uses drop-based plating to identify RBSs that confer sensitivity to spectinomycin (e.g., the approach assumes that a reduction in spectinomycin resistance reflects the expression of an active enzyme). Several “hits” were found (e.g., RBSs that confer sensitivity to spectinomycin) for two recognition sequences (FIG. 49B). This result will be followed by testing the newly identified RBSs in a side-by-side comparison of B2H systems with active and inactive variants of PLpro.

### Example 6: Biosynthesis of Targeted Protease Inhibitors

This example describes using microbial systems to guide the discovery and biosynthesis of natural products that inhibit therapeutic protease targets. One to three pathways that confer a survival advantage by producing inhibitors for each of two disease-relevant proteases may be used. By way of example, natural products were formed that inhibit 3CLpro, PLpro, HIV1pro, WNVpro, DVpro, and USP7.

### Terpenoids

Screening of protease inhibitors began by focusing on terpenoids. This class of natural products was chosen for a number of reasons: (i) Terpenoids include over 80,000 known compounds and represent nearly one-third of all characterized natural products (the basis of approximately 50% of FDA approved drugs); they define a rich molecular landscape for the discovery of bioactive molecules. (ii) Terpenoids can be synthesized and functionalized in E. coli. (iii) A docking study of 3CLpro suggests that it may bind to terpenoids. (iv) Many allosteric sites are only partially solvent exposed and include large nonpolar patches (it was hypothesized that these terpenoids, which are largely nonpolar, might be well suited for finding cryptic allosteric sites, the allosteric site on PTP1B providing a validating example).

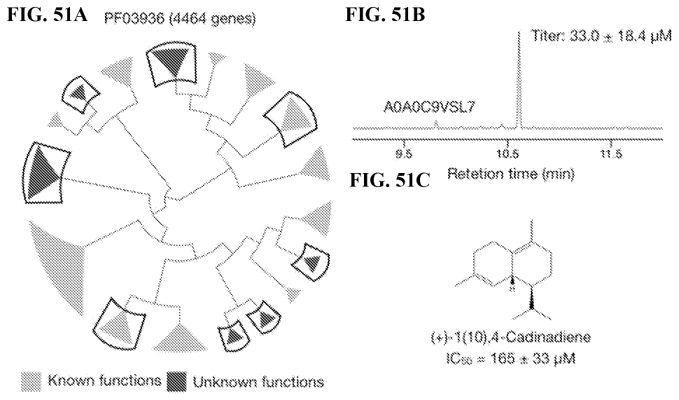

Engineered microbial systems provide a powerful tool for screening genes for their ability to generate enzyme inhibitors. For example, most terpenoids are not commercially available, and even when their metabolic pathways are known, their biosynthesis, purification, and in vitro analysis is a resource-intensive process that is difficult to parallelize with existing methods. The B2H systems offer a potential solution: They can identify inhibitor-synthesizing genes with a simple growth-coupled assay. A PTP1B-specific B2H system was used to screen a diverse set of uncharacterized biosynthetic genes. Briefly, a bioinformatic analysis of the largest terpene synthase family (PF03936) was carried out by building and annotating a cladogram of its 4,464 constituent members; from here, they synthesized three uncharacterized genes from each of eight clades: six with no characterized genes and two with some characterized genes (FIG. 51A). It was reasoned that these 24 phylogenetically diverse genes (8 from fungi, 13 from plants, and 3 from bacteria) might encode enzymes that generate distinct product profiles and, perhaps, novel sesquiterpene scaffolds. Guided by an initial screen, which identified two sesquiterpene inhibitors of PTP1B, similar inhibitors were searched for by pairing each of the uncharacterized genes with the FPP pathway. Intriguingly, six genes conferred a significant survival advantage, and maximal resistance required an active B2H system. One terpene synthase produced mostly (+)-8-cadinene, a structural analogue of an earlier hit with a weaker potency (IC50=165+33 μM; FIGS. 51B-51C). Their ability to detect a weak inhibitor is important indicates that B2H-guided screens have a modest potency threshold, which helps cast a wide net around different starting scaffolds.

In a screen of over 70 terpenoid pathways, several pathways were identified to generate inhibitors of 3CLpro. In other applications, pathways that produce inhibitors of other target enzymes (e.g., HIVpro, PLpro, and USP7) may be used.

The library of biosynthetic pathways was expanded to include a larger set of terpenoid pathways, as well as pathways for non-ribosomal peptides (which include many potent, cell permeable protease inhibitors) and phenylpropanoids (which include inhibitors of flavivirus and coronavirus proteases). In brief, the isopentenol utilization pathway (IUP) was coupled with (i) two prenyltransferases (e.g., farnesyl pyrophosphate synthase [FPPS] or geranylgeranyl pyrophosphate synthase [GGPPS]) and (ii) 37 terpene synthases (e.g., the above 24 genes supplemented with 13 others known to generate structurally distinct products). This library includes 74 pathways and—as estimated—at least several hundred structurally distinct terpenoids (e.g., a single terpene synthase can generate as many as 50 products). IUP was chosen over the mevalonate-dependent pathway because it can generate terpenoids from a cheap precursor (e.g., isoprenol), rather than mevalonate; in liquid culture, it produced amorphadiene (C15) and abietadiene (C20) at titers of 1.88-15.05 mg/L and 121.16-1463.01 mg/L intracellularly (caryophyllene equivalents). These titers are sufficient for the intracellular detection of compounds with IC50s less than or equal to 440 μM (it was assumed that the intracellular concentration must be greater than or equal to the IC50).

The protease inhibitor discovery effort began by focusing on 3CLpro and HIVpro. For each target, the B2H system was used to assess the antibiotic resistance conferred by different pathways (FIGS. 6A-6B). For 3CLpro, several FPP pathways—but no GGPP pathways—conferred a survival advantage. Paradoxically, for HIVpro, two FPP pathways and all GGPP pathways enhanced survival. The surprising influence of the GGPP pathways suggested that either (i) GGPP was an inhibitor of HIVpro or (ii) GGPP caused a stress cellular response that reduces HIVpro activity. Altogether, ten FPP pathways from the first screen enhanced antibiotic resistance for only one B2H system. The target specificity of these pathways suggested that they produce protease-specific inhibitors (as opposed to nonspecific inhibitors, general denaturants, or a general protease-inactivating cellular response).

The large set of pathways identified in the screen of GGPP pathways against HIVpro was intriguing. This result was followed up by (i) measuring the inhibition of HIVpro by GGPP, a potential inhibitor common to all GGPP pathways, (ii) investigating the stress response associated with GGPP production, and (iii) attempting to stabilize HIV protease. HIVpro does not have a positively charged active site, so it was not expected for GGPP to inhibit it. It was hypothesized that a stress response was a more likely cause. Briefly, GGPP can slow the growth of E. coli, and a stress response might inactive HIVpro, which is prone to aggregation. Quantitative proteomics will be performed to compare difference in protein levels between GGPPS-harboring and GGPPS-free strains of E. Coli. Additionally, an attempt to stabilize HIVpro inside the cell by attaching it to fusion partners (e.g., thioredoxin and glutathione-S transferase) was performed; these fusion partners can improve the expression of active soluble protein in E. coli, and they do not interfere with inhibition because they are cleaved off by the protease in the cell.

Intriguingly, two diterpene synthases—O64405 and Q41594 (taxadiene synthase and abietadiene synthase, respectively)—and one monoterpene synthase—UPI0018D1934E (1,8-cineole synthase) conferred resistance when paired with a sesquiterpene precursor. Previous biochemical studies of the two diterpene synthases have shown that they can act on FPP to produce bisabolene- and farnesene-type sesquiterpenes; however, the FPP activity of the monoterpene synthase was unexpected. This finding highlights the value of pairing terpene synthases, which are highly promiscuous, with nonnative precursors (a feat unachievable in screens of natural libraries).

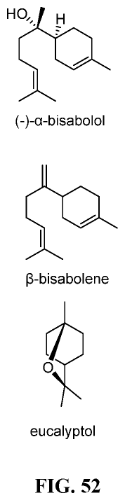

The first screen was followed up by focusing on FPP pathways. In brief, two sets of experiments were performed: (i) Drop-based plating to confirm the survival advantage conferred by each hit (e.g., the terpene synthase and associated precursor pathway). (ii) 10-30 ml cultures to examine the product profiles of each hit. Intriguingly, all ten terpene synthase genes afforded a reproducible survival advantage, but many failed to generate terpenoids in liquid culture. This apparent discrepancy between the results of screens on solid media and terpenoid production in liquid culture may have resulted from differences in strains, precursor pathways, or culture conditions (see below). Nonetheless, for 3CLpro, the three pathways that conferred the greatest survival advantage generated α-bisabolol, β-bisabolene, or eucalyptol as major products (FIG. 52). It was confirmed that α-bisabolol is a potent inhibitor of 3CLpro (IC50=3.33+0.14 μM). In conclusion, the screening methodology was sufficient to identify novel—and in the case of α-bisabolol, unexpected—protease inhibitors.

### Nonribosomal Peptides and Phenylpropanoids

To expand the molecular search space explored in our high-throughput screens, pathways were assembled for nonribosomal peptides and phenylpropanoids. These pathways facilitated the incorporation of heteroatoms (e.g., oxygen, nitrogen, and halogens) at early stages of inhibitor biosynthesis (for terpenoid pathways, the first cyclic molecule is typically a hydrocarbon scaffold). Both sets of molecules also include numerous potent, cell-permeable protease inhibitors (including inhibitors of 3CLpro).

Plasmid-borne biosynthetic routes were chosen for building each new class of natural product. For nonribosomal peptides, nonribosomal peptide synthetases (NRPSs) are identified in bioinformatic analyses of large genomic databanks (e.g., antiSMASH or the NIH Human Microbiome Project). NRPSs are assembly-line enzymes encoded by large gene clusters; they are compatible with expression in E. coli. For phenylpropanoids, one or two plasmids encoding 1-7 bacterial and/or plant genes that convert L-tyrosine or L-phenylalanine to different products were used. Unlike NRPSs, these pathways include discrete enzymes that can be reconfigured to produce different products via combinatorial biocatalysis. Altogether, it was planned to build eight NRPSs and fourteen phenylpropanoid genes that, in various combinations, should have generated over 40 distinct products.

Two carboxylic acid reductases were chosen to study in detail: GupB and Nterp. These enzymes activate two L-tyrosine molecules and reduce them to amino aldehydes, which react to form an unstable imine product that generates a dipeptide pyrazine core (FIG. 53). The enzymes were used to establish a workflow for NRPS assembly, expression, and analysis. Fragments of the Nterp and GupB gene clusters were combined into the final pathway by using Gibson assembly. Like other NRPSs, GupB and Nterp have a thioesterase domain that requires a phosphopantetheinyl group, so these genes were co-expressed with a plasmid harboring the 4′-phosphopantetheinyl transferase (Sfp) from E. coli. A methanol extraction of the cell pellet was used to isolate final products, and the presence of the pyrazine dipeptide was confirmed with HPLC and LCMS (FIGS. 35B-35C). Both gene clusters are functional.

Pathways were assembled for a structurally diverse set of compounds produced from L-phenylalanine or L-tyrosine (FIG. 54). Restriction sites were added to fully assembled apigenin pathway to modularize steps for precursor assembly and scaffold synthesis. Alternative modules were assembled by using the parent plasmid and a small set of additional gene-specific plasmids as templates. Plasmids were constructed with phenylpropanoid-active halogenases to facilitate compound diversification.

### Example 7: Biochemical Characterization of Protease Inhibitors

This example describes using kinetic assays, X-ray crystallography, and in vitro cell studies to characterize new protease inhibitors. Detailed biochemical studies of inhibitors will inform compound optimization efforts that focus on improving potency, solubility, and other drug-like properties. Crystallographic data and cell-based studies of one or more inhibitors may demonstrate a potency supportive of compound optimization (e.g., IC50<5 μM).

### Purification of Proteins and Small Molecules

Terpenoid biosynthesis were scaled up by coupling large-scale liquid cultures with flash chromatography. Amorphadiene (an early indication of an inhibitor of PTP1B) was produced with greater than >200 mg/L from shake flasks and complete purification (>95% purity) within one week.

### Kinetic Studies

Kinetic characterization of α-bisabolol suggested that it was an inhibitor of 3CLpro. FIGS. 30B-30C show the inhibition of 3CLpro activity on a model substrate, a fluorogenic peptide, by a mixture containing α-bisabolol. This terpene alcohol is interesting for three reasons: (i) The IC50 sits at the low end (IC50=3.33±0.14 μM) of lead compounds identified in a previously reported screen of 10,000 compounds-including approved drugs, drug candidates in clinical trials, and other pharmacologically active compounds. This attribute highlights the ability of some embodiments of the methods and systems in this disclosure to sample a novel molecular landscape that may not be effectively explored in other screening methods. (ii) The molecule is a simple terpenoid alcohol and could be valuable for building broad-spectrum antivirals for coronavirus diseases. (iii) It is a relatively small molecule, such that its ligand efficiency is high, and thus it was a promising starting point for lead optimization.

### Biostructural Analyses of 3CLpro

Recombinant 3CLpro was produced in a lab to grow crystals of this protein. X-ray diffraction data was obtained to help complete structural refinement. A 2.1-Å crystal structure of 3CLpro was obtained. Co-crystallization and ligand soaking is used to prepare crystals of the protein-ligand complex. Both approaches have been used in the past, but co-crystallization may be more effective for α-bisabolol, which is nonpolar and may have trouble diffusing to the active site without disrupting the crystal. Crystals of the protein-inhibitor complex may help resolve the mode of inhibition. Proteomics experiments may be performed test a hypothesis that α-bisabolol forms a covalent complex with the catalytic cysteine, of the specific binding site for α-bisabolol.

### Example 8: Assembly of B2H Systems

This example describes a prophetic example for designing B2H systems that incorporate various proteases, in analogous systems disclosed in Example 4: “B2H System with a Protease Recognition Sequence in the Linker”. Two elements integrates into the B2H systems: (i) new protease-specific cleavage sites, and (ii) new viral proteases. Luminescent systems are used, which allow the assessment of both the influence of new cleavage sites on B2H function and the susceptibility of these sites to proteolysis. Starting with functional and operable systems, for example, systems disclosed elsewhere in the present application, the luminescence gene is swapped with a gene for antibiotic resistance. Problematic designs will be screened by alternative RBSs, cleavage sites, and protease expression strategies (e.g., chaperones and/or partial truncations).

It is assessed whether proteolysis of the MidT-RpoZ fusion inhibits B2H activation. The protein-protein interaction that controls expression of the GOI occurs between (i) the kinase substrate (MidT), which is fused to the omega subunit or portions thereof of the RNA polymerase (MidT-RpoZ), and (ii) an SH2 domain, which is fused to 434cI, as shown in FIGS. 39A-39D. Proteolysis of phosphorylated MidT-RpoZ could create MidT fragments that persist in the cell, even after the production of protease inhibitors; if present at a sufficiently high concentration, the proteolyzed MidT domains could inhibit SH2 binding to the intact MidT-RpoZ fusion. Though this has not observed in B2H systems thus far, it may be possible. Without being bound to a particular theory, the lack of observation regarding this effect may be a result of dilution in growing cultures and/or the proteolytic susceptibility of small peptides. If this effect is observed in experimental systems described in this example, a degradation tag72 may be added to the MidT-RpoZ linker, which can reduce the cytosolic lifetime of the MidT domain after proteolysis.

It is assessed whether native E. coli proteases act on the protease recognition sequences. E. coli has native proteases that could act on the cleavage sites present in our B2H systems. This interaction was not observed in B2H systems so far. Without being bound to a particular theory, the lack of observation regarding this effect may be because of the uniqueness of the chosen sites. If the interaction is observed in experimental systems described in this example, alternative protease-specific cleavage sites may be screened or evolved. For the screening methodology, 3-5 alternative sites may be identified from literature. For the evolution methodology, B2H systems that (i) lack a target protease, (ii) contain SpecR as the GOI, and (iii) include sequences with alternative residues flanking the cut site, will be used. First, B2H systems will be screened for growth on spectinomycin to identify sequences that are stable in E. coli. Then, these hits will be paired with target protease in a luminescence-based screen (such as the one depicted in FIG. 3C) to identify target-compatible cleavage sequences.

### Example 9: Multiplexed Screening of Multiple Targets and Many Pathways in Parallel

This example describes a prophetic example using DNA barcoding and next-generation sequencing to parallelize screens of multiple targets and pathways. This example discloses (i) screening ten targets against 100 pathways in a single experiment, and (ii) a set of three potent inhibitors (e.g., IC50<10 μM) for each of five viral proteases.

### Biosynthesis of Protease Inhibitors.

Natural products represent a longstanding source of pharmaceuticals and medicinal preparations. Without being bound to a particular theory, natural products, as a result of their biological origin, tend to exhibit favorable pharmacological properties (e.g., bioavailability and “metabolite-likeness”) and exert a striking variety of therapeutic effects (e.g., analgesic, antiviral, antineoplastic, anti-inflammatory, immunosuppressive, and immunostimulatory). This example describes adding new targets and pathways, and by enhancing the throughput screens.

Disclosed herein are a broad set of modular metabolic pathways for terpenoids, nonribosomal peptides, and phenylpropanoids. These classes include some natural products and protease inhibitors. Also disclosed herein is a library that includes (i) 40 terpene synthases (each of which can be paired with one of three precursor pathways), (ii) 8 nonribosomal peptide synthetases (NRPSs, which can be reconfigured to generate alternative products), (iii) and 23 phenylpropanoid-generating enzymes (e.g., three precursor-enzymes, nine phenylpropanoid enzymes, and 10 tailoring enzymes). This set includes over 100 biosynthetic pathways. The precise number and diversity of possible products is difficult to quantify (a single terpene synthase can produce over 50 terpenoids), but 1,000 is a conservative estimate. This number may seem small in comparison to drug discovery campaigns that begin with libraries having ten million molecules (or more). However, the library disclosed herein are influenced by historical successes and failures, includes only a fraction of potential biologically active molecules, and are typically whittled down to libraries of ˜10,000 likely inhibitors early in the discovery process. The libraries of the present disclosure include a unique set of molecules that are both (i) absent in contemporary libraries (even existing libraries of natural products include molecules pre-optimized by living systems for their own ends) and (ii) biased to be biologically active (e.g., living systems typically use these classes of molecular structures for defense and inter-species signaling). The ability for systems and methods to find a novel inhibitor of 3CLpro, one of the most widely screened enzymes in the world, highlights the advantages of our approach, even when used with relatively small libraries as disclosed herein.

Large-Scale Screens with Many Targets and Many Pathways.

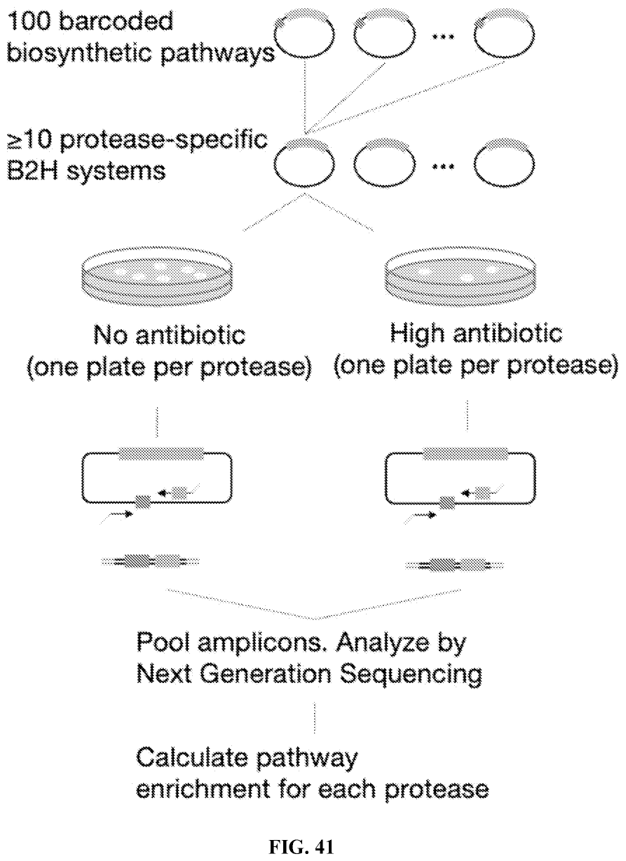

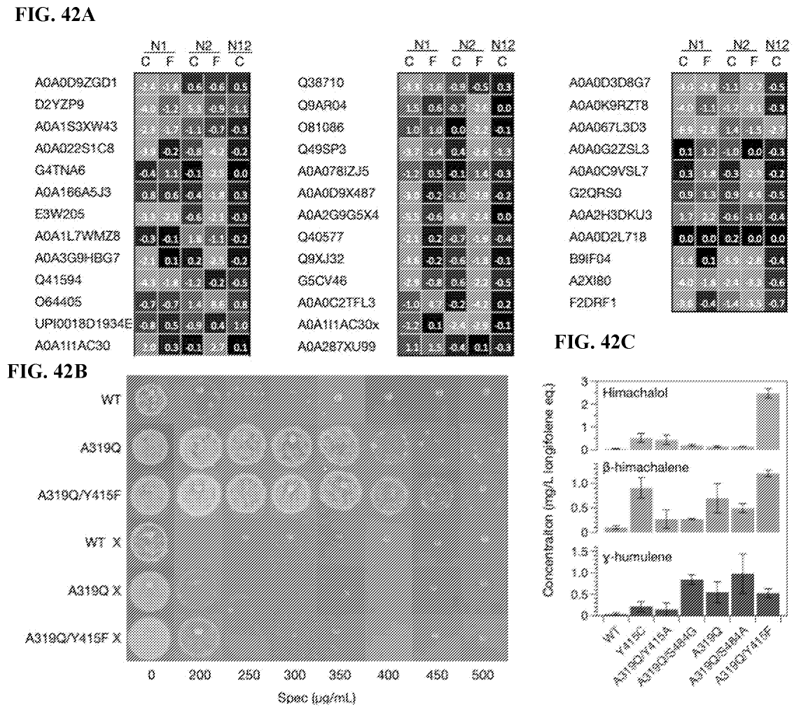

A high-throughput method for screening many targets and pathways in parallel accelerates the discovery of early hits and provide insights about hit selectivity and off-target activity before kinetic assays. The following describes an approach for combining at least ten targets and 100 metabolic pathways into a single screen, as shown in FIG. 41. Each screen includes some or all of the following steps: (i) E. coli is transformed with ten protease-specific B2H systems to create protease-specific strains. (ii) Each strain will be modified with a mixture of at least 100 barcoded biosynthetic pathways. In some cases, a barcode may refer to a unique sequence of at least six bases. (iii) Each transformed strain is cultured on plates with and without spectinomycin. (iv) Each protease-specific plate will be scraped for cells, the cells are lysed, and their barcodes are amplified with primers that contain a second barcode that is specific to both the protease target and the antibiotic condition. All amplification reaction products are combined and next-generation sequencing methods are performed at once on the combination. (vi) The enrichment of each pathway for each target is calculated, similar to the preliminary data shown in FIG. 42A.

### Diversification of Protease Inhibitors.

Enzymes from secondary metabolism facilitate evolutionary adaptation by enabling rapid changes in enzyme function; a single mutation can dramatically alter their substrate specificities and product profiles. This example describes using directed evolution and combinatorial biosynthesis to diversify biosynthetic pathways and broaden early screens (e.g., a barcode could represent a collection of mutated pathways).

Terpenoid pathways are diversified by using directed evolution. For background, the active sites of terpene synthases contain constellations of amino acids that guide catalysis by controlling the conformational space and solvation environment available to reacting substrates. These attributes are modified by using (i) random mutagenesis and (ii) site-saturation mutagenesis (SSM). For SSM, poorly conserved residues located near (<8 Å) the active site are mutated. Resulting mutant libraries will be screened in six steps: (i) The mutant libraries are transformed into B2H-containing E. coli cells. (ii) The transformed cells are plated on solid media with different concentrations of spectinomycin. (iii) Colonies are picked that grow on plates with concentrations of spectinomycin at which the wild-type enzymes do not permit growth. (iv) The terpene synthase genes are sequenced. (v) All hits are verified, and potential background mutations are removed by reintroducing the associated mutations into the starting (e.g., non-surviving) pathway, and by carrying out drop-based plating to retest resistance. The final products are analyzed, purified, and tested as described above. This effort will focus on γ-humulene synthase and epi-isozizaene synthase, which produce many products, FIGS. 42B-42C.

Non-ribosomal peptide and phenylpropanoid pathways are diversified by using domain shuffling and combinatorial biosynthesis. Both efforts focus on the incorporation of non-native tailoring enzymes (e.g., halogenases and cytochrome P450s). Note: Cytochrome P450s, which are membrane-bound, can be challenging to express in bacterial systems. Eukaryotic P450s are expressed in bacterial hosts (e.g., engineering the N-terminal transmembrane helix, co-expression of an appropriate reductase enzyme).

### Characterization of Hits

Compounds generated by pathways that confer a survival advantage are identified and purified by using any one of the relevant method or system disclosed herein. Briefly, flash chromatography and high-performance liquid chromatography (HPLC) purify compounds, and gas chromatography-mass spectrometry (GC-MS) and nuclear magnetic resonance (NMR) spectroscopy are used to identify them. Note: In some cases, compounds may be identified by sampling crude extract (e.g., identification is not dependent on purification).

Potential inhibitors are characterized, and their mode of inhibition is investigated by combining in vitro kinetic assays, X-ray crystallography, and mutational analyses. Briefly, viral proteases are expressed, purified, and crystallized with methods (Table 10). IC50 curves are constructed and crystallographic and mutational studies will be conducted on verified inhibitors.

On-target activity is assessed by using cell-based assays. A wide range of antiviral assays may be employed (e.g., assessment of microscopic cytopathic effects and plaque reduction neutralization tests).

### Library Size

It is assessed if the number of metabolic pathways is sufficient to generate protease inhibitors. It is difficult to estimate the library size required to find an inhibitor of a given target a priori, given the importance of compatibility between library diversity and target structure. A library of the present disclosure produced at least one novel inhibitor of 3CLpro, and the growing collection includes NRPSs that generate peptide aldehydes, a class of molecules that includes potent (IC50˜ 10 nM) inhibitors of serine and cysteine proteases. A peptide aldehyde served as the basis for Bortezomib, an FDA-approved proteasome inhibitor. If initial screening efforts do not yield potent protease inhibitors, the library of biosynthetic pathways may be expanded by adding new genes.

It is assessed if some pathways generate too many products. Highly promiscuous terpene synthases can generate many products but some tend to synthesize only 2-3 major ones (˜50-75% of total). Some examples are 8-selinene synthase and γ-humulene synthase, which convert farnesyl pyrophosphate into ˜30 and ˜50 detectable products, respectively, but only three major products. Inhibitors are isolated from mixtures by using dereplication methods with proteases and phosphatases, which is disclosed elsewhere in the present application.

Strategies for finding inhibitors with improved potencies are assessed. Pathways that generate 1-200 mg/L of natural products are used. At the higher high titers, pathways could produce weak inhibitors at sufficient quantities to inhibit target proteases inside the cell. To improve the stringency of the screen, lower inducer or precursor concentrations can be lowered to reduce inhibitor biosynthesis during drop-based plating. This condition biases the search toward potent inhibitors that function at low concentrations.

### Example 10: Develop a Potent Lead Candidate for Treating COVID-19

Natural products are sometimes considered difficult starting points for pharmaceutical development, in some cases because of their limited natural availability and high synthetic complexity (e.g., compounds with multiple stereocenters). This example describes an approach for identifying molecules having improved potency and drug-like properties over α-bisabolol—and, perhaps, over other 3CLpro inhibitors identified—for the treatment of COVID-19. Contemplated herein is an inhibitor having an IC50 of <100 nM. This IC50 can be sufficient for some animal studies and exhibits a 30-fold improvement over the initial IC50. This example also describes a workflow for the (bio) synthetic optimization of hits identified with a platform disclosed herein. This work seeks to develop a general (bio) synthetic workflow for progressing early-stage hits into drug-like compounds.

### Mechanistic Role of 3CLpro and PLpro in Coronavirus

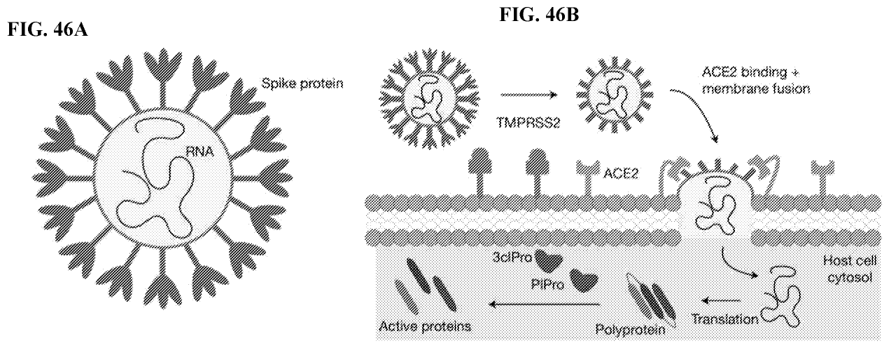

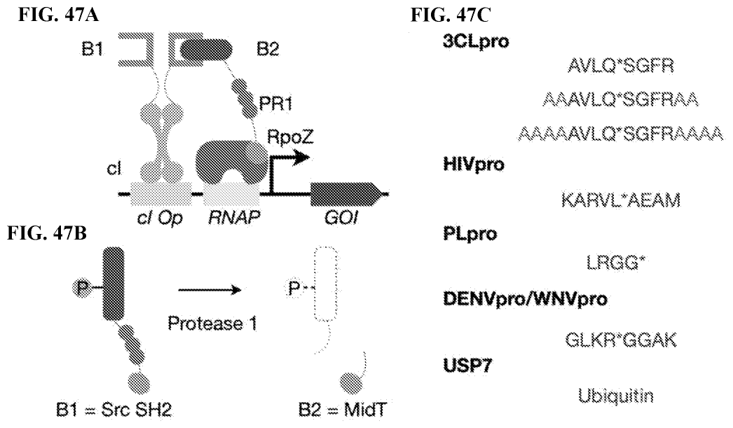

Coronaviruses contain a single-stranded positive-sense RNA genome encased in a membrane envelope, as illustrated in FIG. 46A. Glycoprotein spikes, which sit on the surface of the envelope, give it a crown-like appearance. There are four genera of coronaviruses: alpha, beta, gamma, and delta. The β-coronaviruses include severe acute respiratory syndrome virus (SARS-CoV), Middle East respiratory syndrome virus (MERS-COV), and SARS-COV-2. SARS-COV-2, like the others, attacks the lower respiratory system, causing viral pneumonia. It can also impact the gastrointestinal system, heart, liver, kidney and central nervous system.

The SARS-COV-2 genome encodes 16 non-structural proteins, 9 accessory factors, and 4 structural proteins. As illustrated in FIG. 46B, infection begins when a serine protease (TMPRSS2) of host origin primes the glycosylated spike(S) protein of the virus for binding to angiotensin-converting enzyme 2 (ACE2), a host receptor required for viral invasion. Upon entering a cell, the virus releases its RNA genome, which is translated into viral proteins by cell machinery. The coronavirus main protease (3CLpro) and the papain-like protease (PLpro)—both cysteine proteases—cleave viral polyproteins into functional subunits, and the viral RNA-dependent RNA polymerase (RdRp) synthesizes new copies of genomic RNA. The activities of TMPRSS2, 3CLpro, and PLpro are important for viral infection and promising targets for antiviral therapeutics.

### Improvement of the Potency and Drug-Like Properties of a Sesquiterpene Alcohol.

As disclosed elsewhere herein, α-bisabolol was found to inhibit 3CLpro. Briefly, α-bisabolol is an unusual—and, possibly, covalent—inhibitor; whereas some of the other 3CLpro inhibitors in clinical development are peptide mimics. A crystal structure of 3CLpro bound to α-bisabolol will be collected, evaluated for off-target activity against human cysteine proteases (e.g., Cathepsin L and B), and be tested for cell-based antiviral activity with IAR.

Clinical candidates may be developed in stages: (i) hit identification, (ii) hit-to-lead optimization, and (iii) lead optimization (in broad terms). A promising hit often possesses at least five features to enter hit-to-lead optimization: (i) single-digit micromolar potency or less, (ii) a crystal structure of the protein-inhibitor complex to guide synthetic chemistry, (iii) a strategy for chemical functionalization, (iv) a readily synthesizable core structure, and (v) several alternative structures as backups. Features (iii)-(v) may hinder the progression of natural products into promising candidates; natural products can have low natural titers and complex chemistries.

Though α-bisabolol is commercially available, most structural variants are not. Some examples of structural variants are shown in FIG. 43. The ability of terpene synthases to form asymmetric carbon-carbon bonds to produce structural variants in E. coli (and purify them as described above) are assessed. This structure series illuminates the molecular determinants of binding, guide compound optimization, and demonstrate the value of fermentation for the rapid production of alternative cores.

Synthetic chemistry and enzymatic functionalization are combined to improve the potency and drug-like properties of potent cores, starting with α-bisabolol. Inspired by plant secondary metabolism, cytochrome P450 enzymes will be used to selectively hydroxylate unactivated carbon-hydrogen bonds. An enzyme panel that includes human, insect, and plant P450s is screened. Note: Human P450s can selectively functionalize (+)-epi-α-bisabolol and likely accept α-bisabolol as a substrate. For the screening effort, three strategies are pursued: (i) a B2H screen, (ii) whole cell biocatalysis, and (iii) biocatalysis with purified membrane fractions. The first approach could identify potency-enhancing functional groups in growth-coupled assays (e.g., selection); the latter two will use GC-MS and LC-MS to identify functionalized molecules. This work may resolve enzymatic schemes for the diversification and optimization of terpenoids.

Improved inhibitors will be characterized (e.g., more potent or more soluble inhibitors) with cell-based assays and in vitro absorption, distribution, metabolism, and excretion (ADME) studies. Briefly, cell-based assays and ADME studies may be conducted.

Upon identifying an inhibitor with a potency less than 100 nM, an animal study is used to evaluate bioavailability, pharmacokinetics, and basic toxicity.

### Example 11: Solid-Media Testing Methodology

In some embodiments of high-throughput screens, S1030 cells are transformed with a B2H system, an isopentenol utilization pathway, and a terpene synthase, and are grown on solid media (LB agar plates). The S1030 cells lacked the RpoZ subunit of RNA polymerase; the isopentenol utilization pathway allowed for modulation of terpenoid production by controlling the concentration of isoprenol, an essential precursor; and the LB agar improves the stringency of the screen. To follow up on interesting hits, DH5α or DH10B cells were transformed with a complete mevalonate-dependent isoprenoid pathway (e.g., one that generates FPP or GGPP from acetyl-CoA) and a terpene synthase and were grown in 0.01-1 L of liquid TB media. The complete isoprenoid pathway reduces the cost of high-titer expression by avoiding the use of exogenously added substrates, and the strains (e.g., DH5α and DH10B) and media improve titers. Moving forward, an intermediary step may be added in which the terpenoid profile will be analyzed directly from the solid media (e.g., the screen).

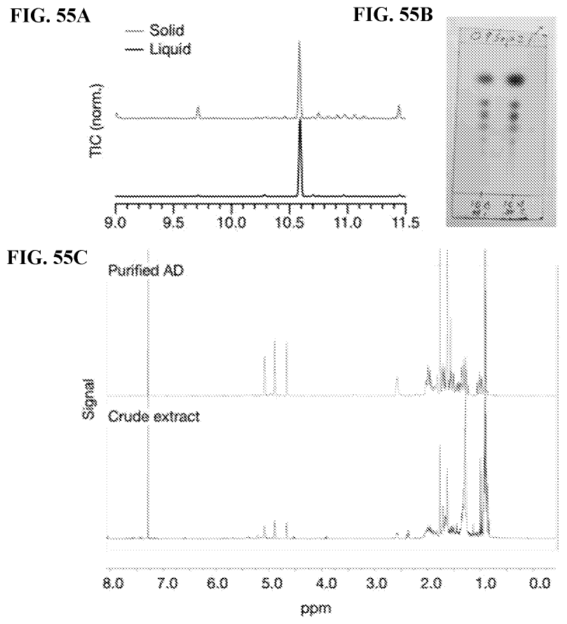

A procedure for testing solid media was developed: A small section of the agar was removed, cells were lysed, and hexane overlay was used to extract a sample for GC-MS; this approach yielded detectable terpenoids (FIG. 55A). To permit use of the same strain in the screen and scale-up, two approaches are pursued: (i) The RpoZ subunit of RNA polymerase were removed from the DH5α and DH10B strains, and (ii) the B2H systems were modified by swapping in the alpha subunit of RNA polymerase in place of the omega subunit (α-based systems do not require a genomic deletion). Both approaches allow for the use of DH5α and DH10B strains of E. coli in the screen. Note: Media conditions between the screen and scale-up may still differ, but the intermediary analysis step will allow the determination if those differences affect product profile.

### Example 12: Verifying Target Inhibition by Pathways

The screening approach allows identifying pathways that confer a survival advantage in the presence of a B2H system. This advantage may result from target inhibition, but this connection may be tested in additional ways. In vitro kinetic assays can be used to carry out such tests.

### Purification of Proteins and Small Molecules

In brief, 3Clpro was expressed with both (i) an N-terminal GST tag (which is cleaved by the protein during expression) and (ii) a C-terminal polyhistidine tag (which facilitates purification with nickel-affinity chromatography). A precision protease was used to remove the C-terminal tag prior to anion exchange. This protocol yielded titers of purified protein (˜10 mg/L) sufficient for in vitro kinetic assays and X-ray crystallography. Notably, similar protocols, which yield proteins without expression artifacts (e.g., tag or linker), are compatible with the other target proteases explored in this proposal.

Purified terpenoids were produced by coupling large-scale liquid cultures with chromatographic separation. 1-L liquid cultures in high-yield flasks were cultured for 2-4 days, after which (i) a hexane overlay was used to extract all terpenoids, (ii) vacuum liquid chromatography was performed for initial separation, and (iii) flash chromatography (normal or reverse phase silica) was performed to isolate products of interest. At various steps in this process, 1H NMR, GC-MS, and thin layer chromatography (TLC) was performed to monitor purification. Analysis of amorphadiene highlights the steps the results afforded by these methods (FIGS. 55A-55C).

### Kinetic Studies

Förster resonance energy transfer (FRET) peptides provide a facile means of assaying protease inhibitors. These peptides contain a fluorophore and a quencher separated by a protease recognition domain; peptide cleavage separates the fluorophore and quencher and increases fluorescence.

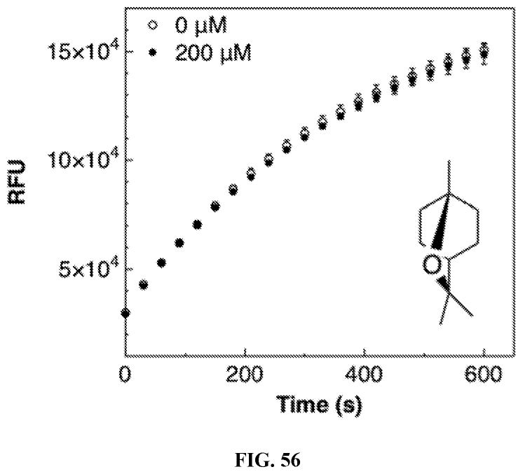

For 3CLpro, a commercially available substrate was used: Mca-AVLQSGFRK(Dnp)K (SEQ ID NO: 1), where Mca ((methoxycoumarin-4-yl) acetyl) was the fluorophore and 2,4-dinitrophenyl (Dnp) was the quencher. Using this assay, inhibition by eucalyptol and α-bisabolol (FIGS. 30B-30C, FIG. 56) was examined. Intriguingly, eucalyptol showed no inhibitory effect—an example of a false positive; however, α-bisabolol was a potent inhibitor (IC50=3.33±0.14 M; FIG. 30C). This molecule is extremely interesting for three reasons: (i) its IC50 was at the low end of lead compounds identified in a previously reported screen of 10,000 compounds—including approved drugs, drug candidates in clinical trials, and other pharmacologically active compound, (ii). the molecule is a simple terpenoid alcohol and could be valuable for building broad-spectrum antivirals for coronavirus diseases, and (iii) It is small (e.g., its ligand efficiency is high) and thus a promising starting point for lead optimization.

### Biostructural Analyses of Inhibitors

The inhibitory mechanisms of newly discovered hits are analyzed by collecting X-ray crystal structures of protein-inhibitor complexes. These structures can reveal (i) the contribution of protein-ligand contacts (hydrogen bonds, halogen bonds, and van der Waals contacts) to differences in binding affinity and (ii) new modes of covalent inhibition.

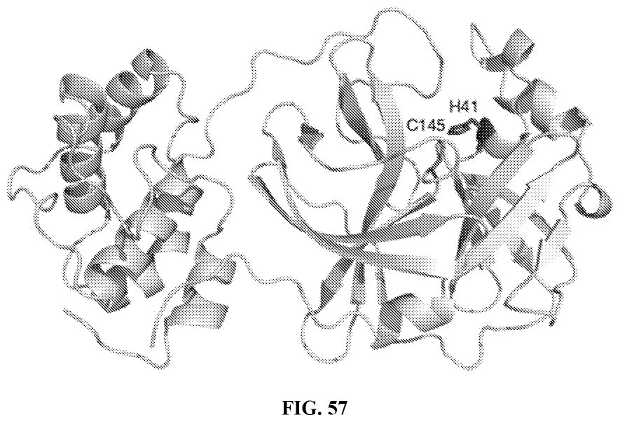

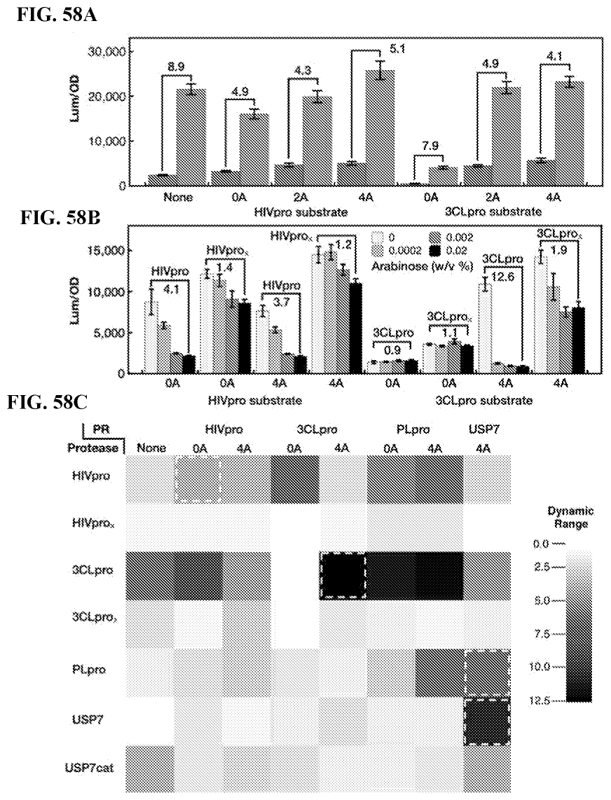

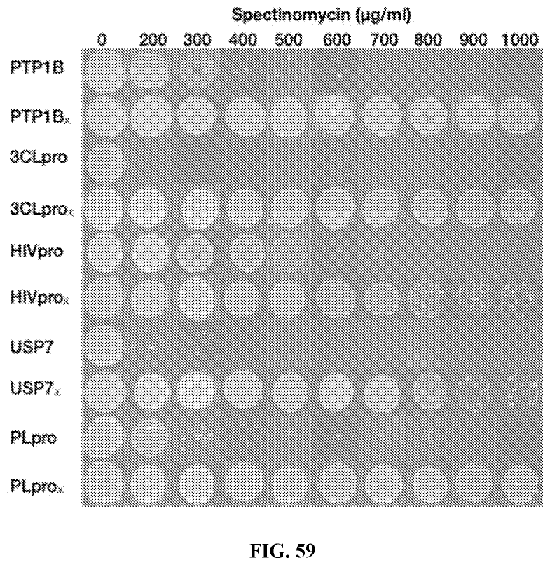

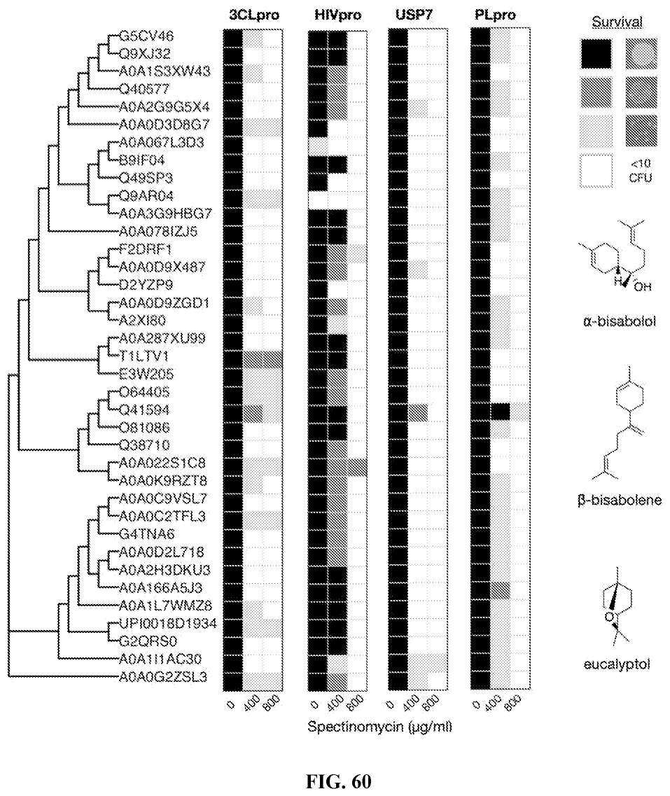

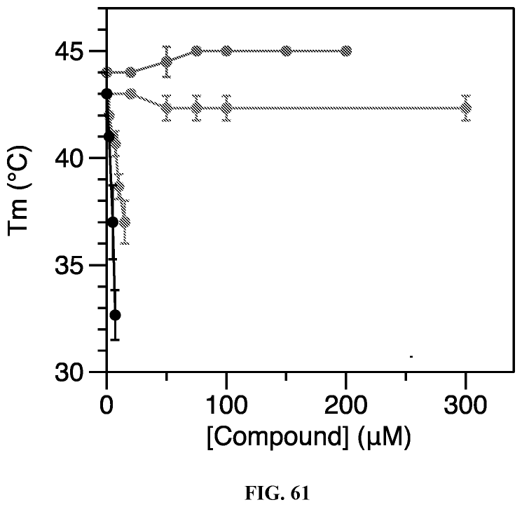

Biostructural analyses began with 3CLpro. The asymmetric unit of this enzyme forms one polypeptide, which associates with another polypeptide to form a crystallographic two-fold axis of symmetry. Inhibitors of 3CLpro can be co-crystallized or soaked into ligand-free crystals. This enzyme has 3 domains: domain I (8-101), domain II (102-184), and domain III (201-303). The Cys-His catalytic dyad and substrate-binding cleft sit between domains I and II. Previous covalent inhibitors of 3CLpro have involved the formation a covalent adduct with the catalytic cysteine (C₁₄₅); however, unlike N3—a well characterized covalent inhibitor—α-bisabolol is not a Michael acceptor. A variety of crystallization buffers were screened and a structure of 3CLpro was collected, shown in FIG. 57. Co-crystallization and ligand soaking will be used to prepare crystals of various protein ligand complexes for beamtime.

### In Vitro Cell Studies.

Cell-based assays of 3CLpro inhibitors is described herein. It begins with a plaque assay, which quantifies the plaques formed in cell culture upon infection with serial dilutions of a virus, which is the standard methodology for quantifying concentrations of replication-competent lytic virions. Neutral red is used to stain monolayers of mammalian cells to look for differences in plaque formation associated with α-bisabolol treatment. Next, real time quantitative PCR (RT-qPCR) is be used to measure viral yield reduction in cells treated with α-bisabolol. For both studies, Vero E6 cells is used.

Cell-based assays for USP7 inhibitors are described herein. For background, USP7 is an important regulator of MDM2, an E3 ligase that promotes proteosomal degradation of the tumor suppressor p53. Human colon cancer cells (HCT 116) are treated with increasing concentrations of inhibitor, lyse them, and use a ubiquitin-propargylamine (Ub-PA) probe is used to measure on-target engagement (e.g., this probe should compete with the inhibitor binding to USP7 but not USP47 or other off-target USPs212). Next, a similar experiment is performed, but Western Blots are performed to examine the influence of inhibitors on downstream signaling targets. In particular, concentration-dependent decrease in MDM2 and increase in p53 and p21 will be examined. This analysis will help to establish on-target activity in mammalian cells.

### Example 13: Selection of an Initial Bacterial Two-Hybrid (B2H) System

A bacterial two-hybrid (B2H) system in which a phosphorylation-mediated binding event activates transcription of a GOI (as shown with respect to FIG. 79A) was developed and used to detect phosphatase inhibitors. The B2H system described above was used to select an initial Transcription-based detection system. Transcription-based detection systems are advantageous because transcription, translation, and biocatalytic production of a signal (if the gene product is an enzyme) are amplification reactions; at each step, a single biomolecule (e.g., one gene, mRNA, or enzyme) can generate many more, allowing a single transcriptional activator to produce a signaling cascade.

A general architecture to detect protease inhibitors was selected. Two protein fusions formed the core of the base B2H: (i) a Src homology 2 (SH2) domain fused to the cI repressor, and (ii) a kinase substrate domain (MidT) fused to the omega subunit of RNA polymerase (RpoZ). Src-mediated phosphorylation of the substrate domain allowed the substrate domain to bind to the SH2 domain, and the resulting substrate-SH2 complex activated transcription of the GOI by localizing RNA polymerase to its promoter; PTP1B-mediated dephosphorylation of the substrate, in turn, prevents activation. 3CLpro overexpression was found to reduce GOI transcription (as shown with respect to FIGS. 83A-83D). 3CLpro was used to evaluate two different binding pairs: a phosphorylation-dependent pair (SH2/MidT, as shown with respect to FIG. 83A) and a phosphorylation-independent pair (SH2ABL/HA4, as shown with respect to FIG. 83B). Briefly, 3CLpro was placed on an arabinose-inducible plasmid and each pair-specific B2H system was tested with a luminescent reporter (GOI=LuxAB). For both systems, overexpression of an active protease—but not an inactive protease—reduced luminescence. The phosphorylation-independent pair yielded a larger dynamic range (e.g., DR, the change in luminescence caused by protease expression; 10.7±1.2 vs. 6.3±0.9, as shown with respect to FIG. 83C) but a higher background signal. When the GOI was swapped with a resistance gene (SpecR), this high background allowed the inactive B2H to confer substantial antibiotic resistance (with respect to FIG. 83D). Sensor development with the phosphorylation-dependent binding pair was continued.

### Example 14: Development of B2H Systems that Detect Protease Inhibition

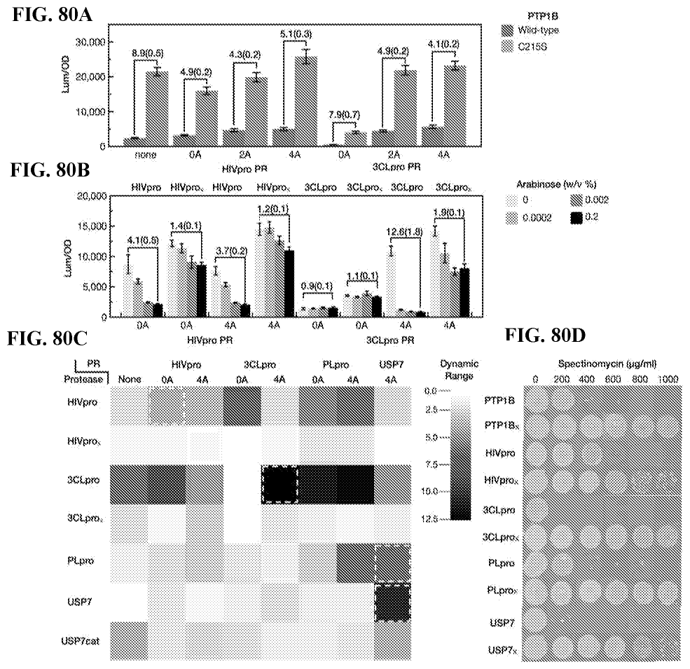

A fusion of a protease recognition (PR) site to the substrate-RpoZ fusion by adding PR sites for HIVpro and 3CLpro, each flanked by 0-4 alanine residues (as shown with respect to FIG. 79) was used in order to render the fusion susceptible to proteolysis and enhance the DR of the B2H system of Example 13. These sites reduced the DR of luminescent systems by up to two-fold but retained the desired B2H activity (e.g., PTP1B inactivation increased luminescence; FIG. 2A). Active proteases reduced luminescence for both HIVpro systems tested (0- and 4-alanine versions) but only one 3CLpro system (the 4-alanine version; FIG. 80B), and the inclusion of PR sites improved DR (as shown with respect to FIG. 80C). Inactive proteases also caused a slight reduction in luminescence (as shown with respect to FIGS. 80B and 80C), which could reflect binding of these proteases to PR sites (an interaction that could disrupt B2H function) or a general cellular response to protein overexpression.

Luminescent B2H systems were used to screen different combinations of proteases and protease-specific cleavage sites (as shown with respect to FIG. 80C) by adding PR sites for HIVpro, 3CLpro, PLpro, and USP7 to the substrate-RpoZ fusion and evaluating the response of each system to protease overexpression. While both PLpro and USP7 remove ubiquitin, PLpro recognizes its terminal LRGG sequence, 57 and thus, PLpro was included as a separate site. Additionally, for USP7, the catalytic domain was included with and without a C-terminal extension that stabilizes its catalytically competent conformation. 3CLpro, PLpro, and USP7 reduced luminescence most significantly when paired with cognate PR sites, while HIVpro appeared functional with all sites, which was behavior consistent with its reported promiscuity. The extended version of USP7 yielded the highest dynamic range. The native PR sites (with 0- or 4-alanine flanking regions) were selected to complete B2H development.

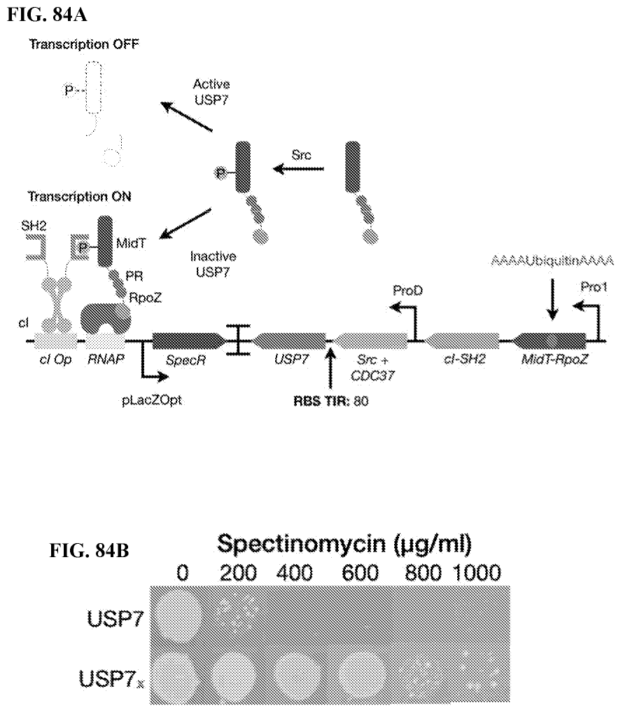

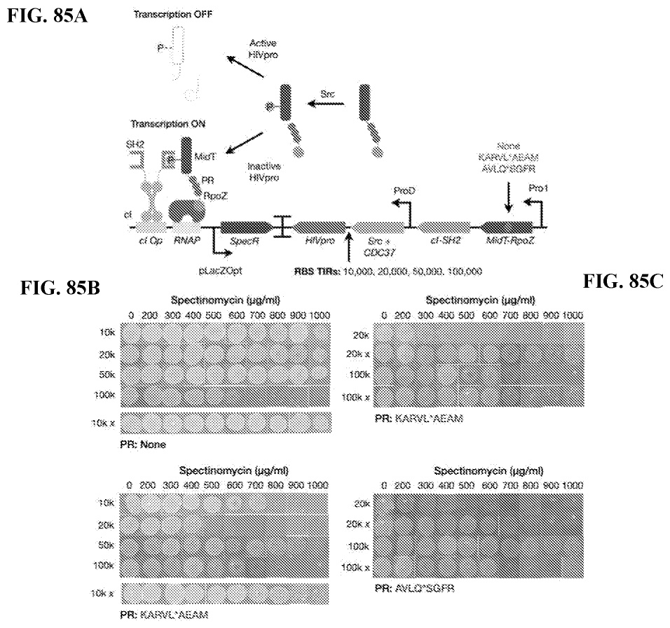

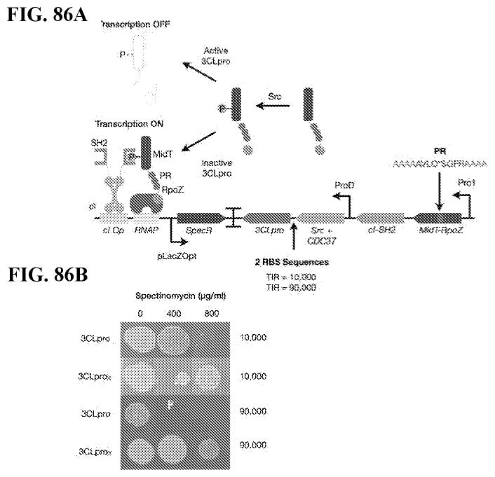

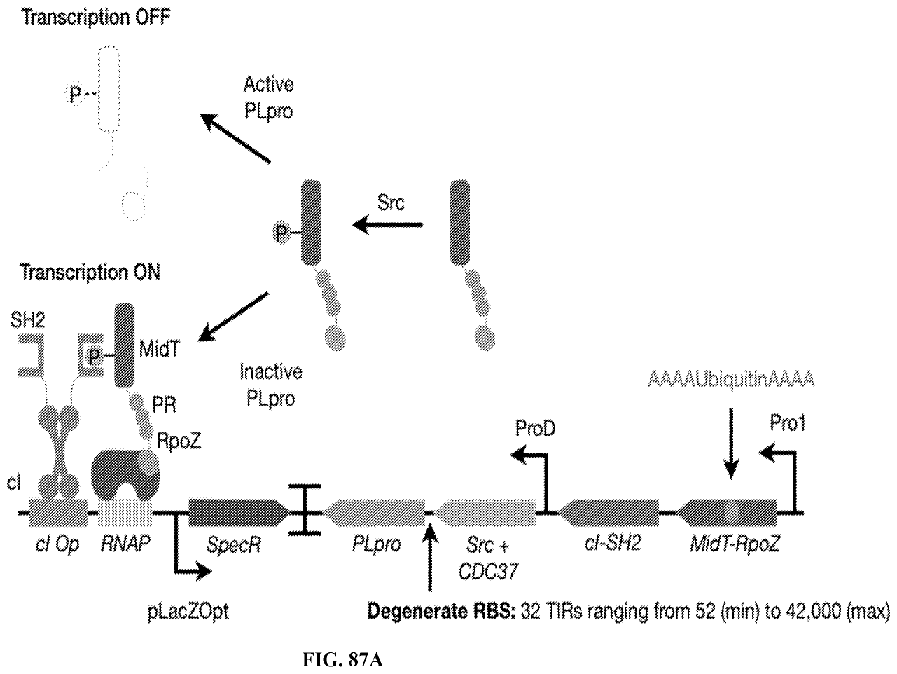

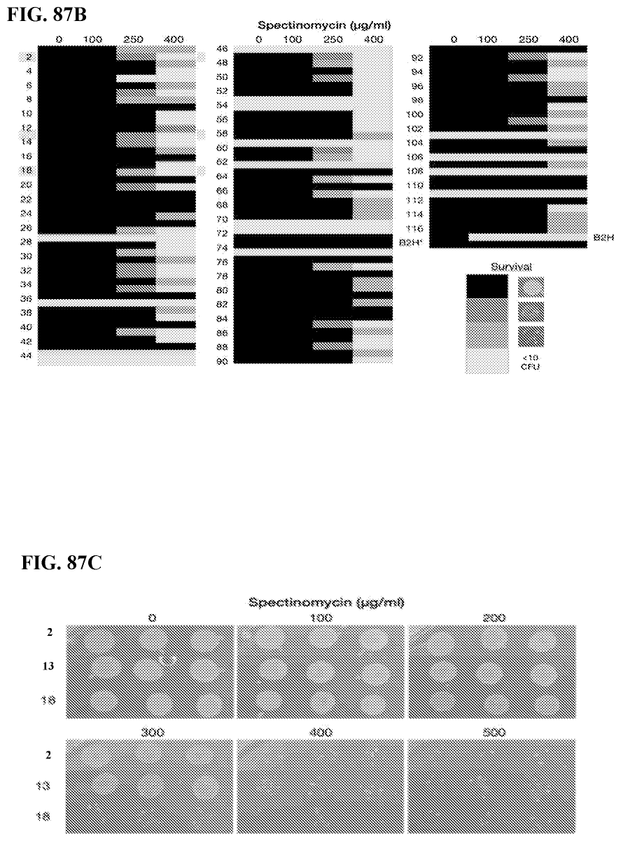

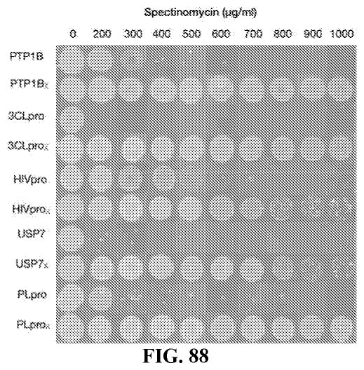

Single-plasmid B2H systems were constructed by making three modifications to the base system (i) exchanging the gene for PTP1B with protease genes, (ii) adding the PR sites selected in the luminescent screen, and (iii) exchanging the luciferase gene (LuxAB) for a spectinomycin resistance gene (SpecR). The B2H for USP7 worked immediately (FIGS. 84A-84B), as active USP7 conferred sensitivity to spectinomycin (e.g., death at 200 μg/ml spectinomycin), and inactive USP7 yielded resistance (e.g., growth at 800 μg/ml spectinomycin). By contrast, B2Hs for the remaining proteases conferred mild sensitivity to spectinomycin. The insensitivity may have resulted from poor protease expression, and thus, the ribosome binding sites (RBS) were modified for protease genes. Relatedly, HIVpro and 3CLpro, a screen of several RBSs with different translation initiation rates (TIRs) yielded variants that enhanced sensitivity to spectinomycin (as shown with respect to FIGS. 85-86). For PLpro, the screen of alternative RBSs was streamlined by using degenerate primers to build a library of 32 variants in a single amplification reaction (FIGS. 87A-87C). The final B2H systems, equipped with optimized RBSs, linked protease inactivation to a major improvement in spectinomycin resistance (as shown with respect to FIGS. 80D and 88).

### Example 15: Biosynthesis of Terpenoids

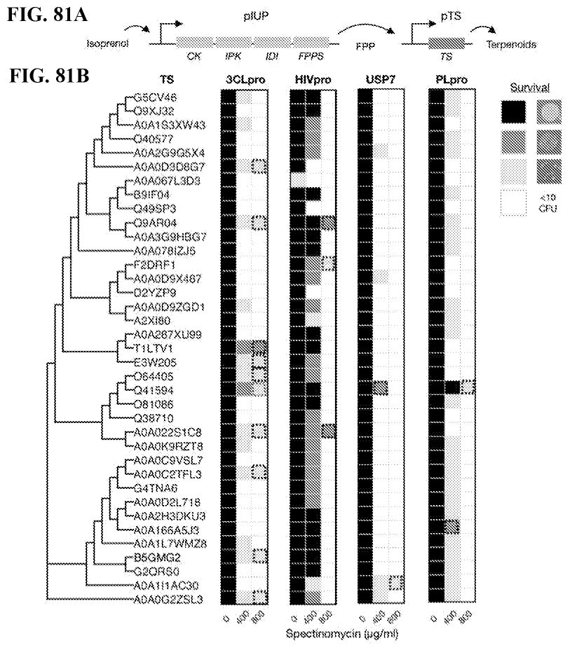

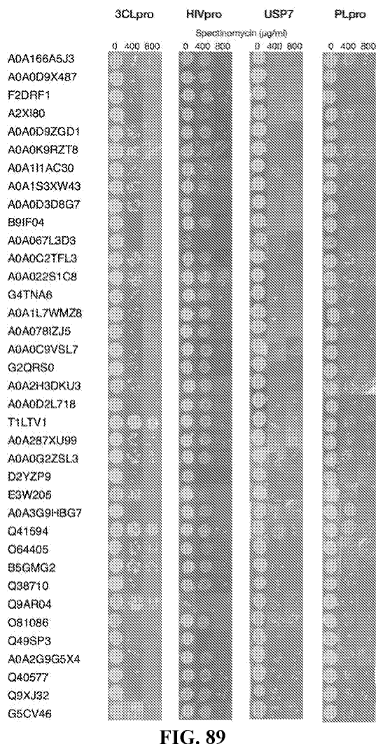

The B2H systems of example 13 were evaluated to find unexpected inhibitors by using them to screen terpenoid pathways, as the pathways generate mixtures of products that are challenging to purify. As nonpolar molecules, terpenoids are also scaffolds for building protease inhibitors, which are typically peptide mimics. Briefly, each terpenoid pathway was assembled with two plasmid-borne modules, (1) pIUP, which converts isoprenol to farnesyl pyrophosphate (FPP), and (2) pTS, which encodes a terpene synthase (TS, as shown with respect to FIG. 81A). By swapping out the second module, 37 phylogenetically diverse TSs were examined (as shown with respect to FIG. 81B). This set of 37 TSs was selected based on analysis of eight clades from the largest TS gene family (PF03936) and includes TSs shown to act on C10, C15, and C20 linear isoprenoids (geranyl pyrophosphate, or GPP; FPP; and geranylgeranyl pyrophosphate, or GGPP, respectively), as well as uncharacterized TSs. Due to TSs being notoriously promiscuous, it was speculated that some of the enzymes might generate products from nonnative substrates (e.g., FPP). Pathways that improved the antibiotic resistance of B2H-encoded E. coli were searched for in order to carry out the screen. In the first screen, the 3CLpro-specific B2H yielded the most hits (as shown with respect to FIGS. 81 and 89). Since 3CLpro is an important target for treating COVID-19 and other coronavirus diseases, 3CLpro was focused on for the remainder of the analysis.

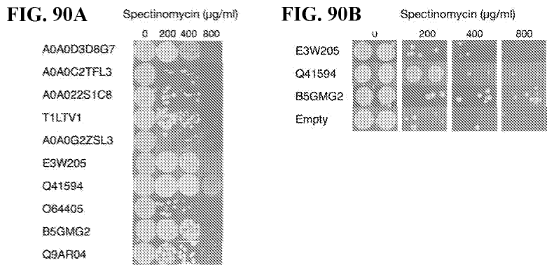

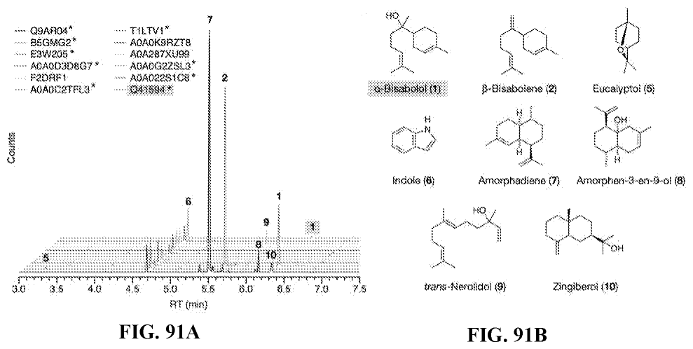

The initial hits were refined through two steps. First, product profiles in liquid culture were examined by pairing each TS with a plasmid harboring the mevalonate-dependent isoprenoid pathway from Saccharomyces cerevisiae (pAM45), which pathway afforded high titers of sesquiterpenes in E. coli and, thus, facilitated TS characterization. Of nine initial hits, five generated products detectable with GC-MS (with respect to FIGS. 91A-91B). The unproductive TSs were either false positives or have in vivo productivities that are highly sensitive to culture conditions. Next, all hits were re-screened in biological triplicate (regarding FIGS. 90A-90B). One yielded a particularly consistent survival advantage (e.g., reproducible growth at higher antibiotic concentrations than other TSs): Q41594, a taxadiene synthase from Taxus brevifolia. Q41594 natively converts GGPP to taxadiene, but it can generate bisabolenes from FPP. With this system, it produced a small amount of α-bisabolol (FIGS. 82A-82B).

In vitro kinetic assays allowed for examination of the inhibitory effect of α-bisabolol. The small amount of α-bisabolol produced by Q41594 in liquid culture was difficult to purify, so three commercially available diastereomers ((−)-α-bisabolol, (+)-α-bisabolol, and (+)-epi-α-bisabolol) were tested. All three diastereomers had similar IC50s, which ranged from 30±5 μM to 80±37 μM, a range consistent with the Kis of compounds identified with previous genetic screens (regarding FIGS. 82D and 92). These IC50s confirm that Q41594 can generate inhibitors of 3CLpro but were surprising for two reasons: (i) they were higher than the intracellular titer of α-bisabolol produced by Q41594 in liquid culture, and (ii) they were similar to—or lower than—the intracellular titer afforded by E3W205, a β-bisabolene synthase that confers less antibiotic resistance than Q41594 (FIG. 82E). Further, intracellular titers can vary with media conditions (as observed between LB and TB liquid media, as shown with respect to FIG. 82E) and may differ on agar plates. Nonetheless, the lack of a simple correlation between α-bisabolol production and antibiotic resistance indicates that α-bisabolol, alone, is insufficient to explain the survival advantage afforded by Q41594.

TSs may exhibit different toxicities in E. coli, even in the absence of isoprenoid pathways and/or active B2H systems, which may be a result of differences in protein expression or solubility. To evaluate the contribution of TS toxicity to the fitness advantage conferred by Q41594, E. coli was transformed with plasmids harboring Q41594, E3W205, and O64405 (e.g., a hit and two non-hits) and grew the transformed strains in liquid culture (regarding FIG. 82F). The Q41594 strain exhibited the lowest specific growth rate, an indication that this TS does not improve antibiotic resistance by reducing protein toxicity.

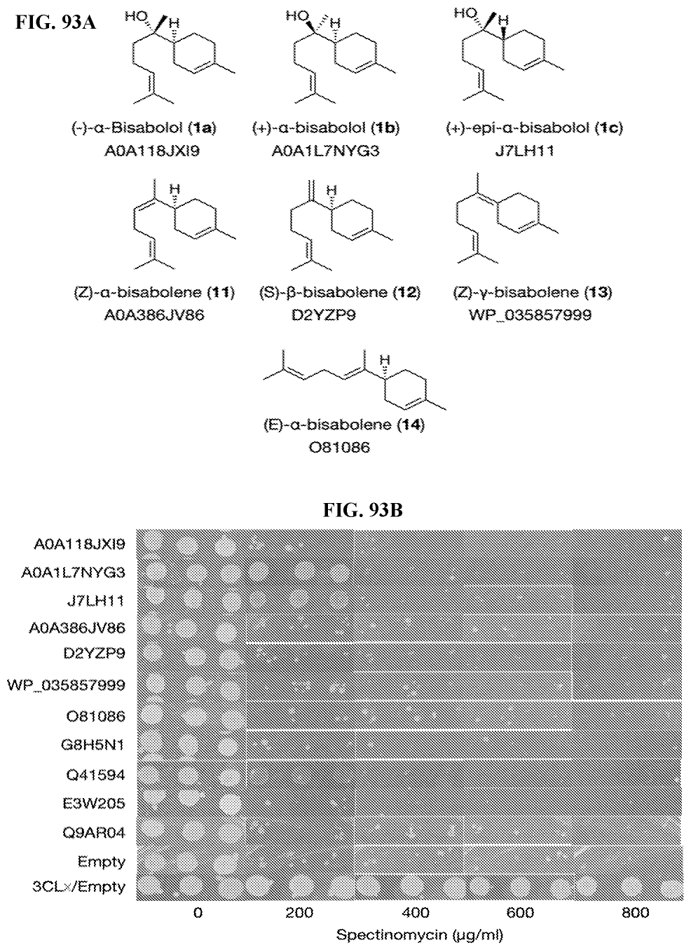

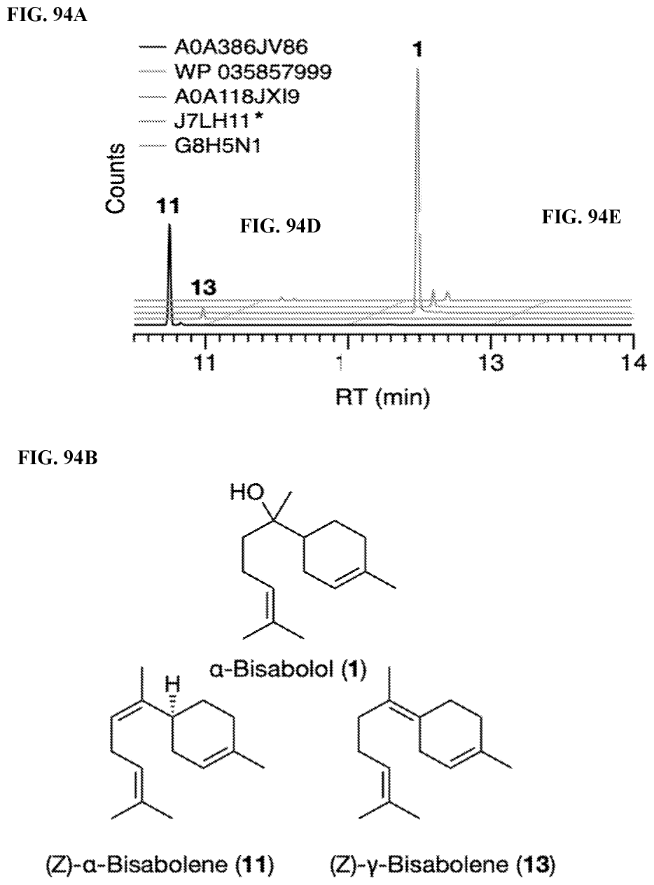

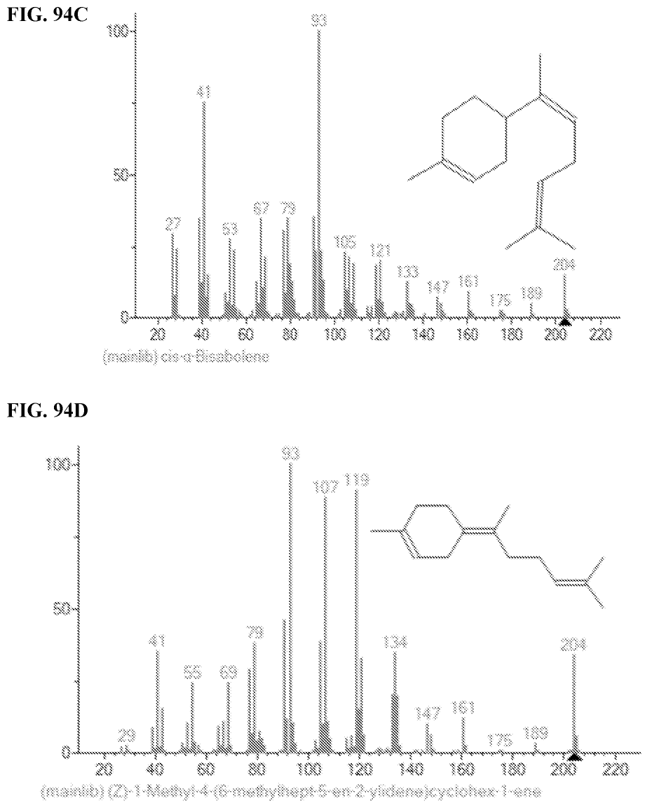

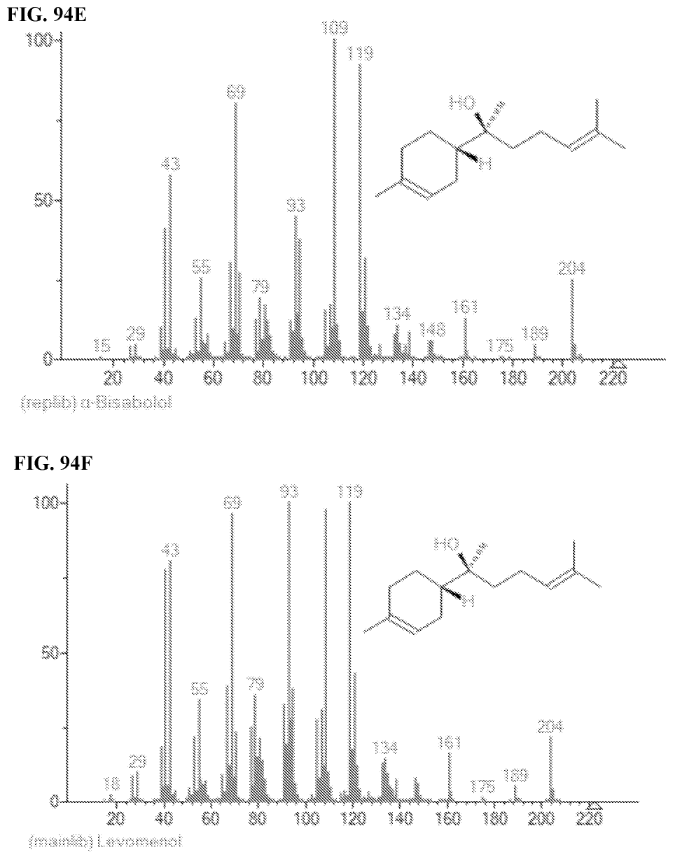

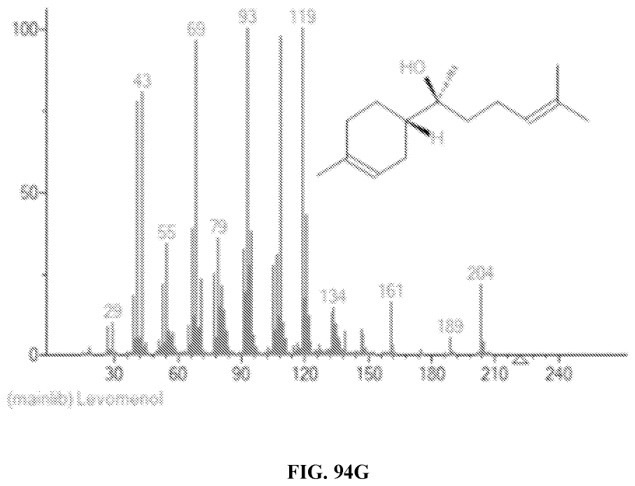

A handful of well-characterized TSs produce α-bisabolenes. These enzymes were used to carry out a systematic analysis of the link between α-bisabolol production and antibiotic resistance. In a B2H screen of seven additional TSs, two α-bisabolol producers emerged as hits: (i) A0A1L7NYG3, a (+)-α-bisabolol synthase from Artemisia kurramensis, and (ii) J7LH11, a (+)-epi-α-bisabolol synthase from Phyla dulcis (FIG. 93). Similar to the first screen, however, the product profile was an imperfect predictor of survival advantage. Two other α-bisabolol producers failed to improve resistance: A0A118JX19, a (−)-α-bisabolol synthase from Cynara cardunculus, generated more α-bisabolol than J7lH11, and (ii) G8H5N1, a sesquiterpene synthase from Solanum habrochaites, produced less (FIGS. 94A-94G). The discrepancy in resistance afforded by TSs that produce α-bisabolol suggests that several of these TSs make an undetectable product that inhibits 3CLpro. This mechanism seems unlikely, given the necessary potency of a minor product (e.g., a low intracellular concentration requires a low IC50) and the requirement that it is generated only by TSs that also make α-bisabolol. Alternatively, survival might require enough α-bisabolol to inhibit 3CLpro but not so much that α-bisabolol inhibits other enzymes in the cell and disrupts growth. This theory assumes that α-bisabolol is a general inhibitor, a behavior consistent with both the emergence of Q41594 as a hit for multiple proteases (FIGS. 81A-81B) and the inhibitory effects of other terpenoid alcohols; it also implies that the production of genera inhibitors as side products could be problematic. This theory is consistent with the improved resistance afforded by Q41594 and J7LH11 over A0A118JX19, which produces more α-bisabolol, and E3W205, which has a prominent side product. A rigorous test of this theory, however, requires an analysis of a large number of α-bisabolol synthases with different productivities and product profiles.

In vitro analysis was completed by examining the inhibitory effects of three other bisabolenes produced by TSs included in the screen (as shown with respect to FIG. 82C): α-bisabolene, β-bisabolene, and β-bisabolol. For this test, stereoisomers that were purchasable or easily purify from liquid culture were selected. Intriguingly, α-bisabolene was less inhibitory than α-bisabolol (IC50=186±12 μM), and the inhibitory effects of β-bisabolene and β-bisabolol were too weak to yield accurate IC50 estimates (≤50% inhibition at 1000 μM in our initial screen, regarding FIG. 92). These results are consistent with the ability of α-bisabolol, a major product of all three hits identified in our screen, to improve the antibiotic resistance of B2H-encoded cells by inhibiting 3CLpro.

Materials for carrying out the methods as described in Examples 13-14 may include M9 minimal salts, tris(2-carboxyethyl) phosphine (TCEP), bovine serum albumin (BSA), phenylmethylsulfonyl fluoride (PMSF), 3-methyl-2-buten-1-ol (prenol), dimethyl sulfoxide (DMSO), isopropyl-D-thiogalactopyranoside (IPTG), (−)-α-bisabolol, 3CLpro fluorogenic peptide substrate (TSAVLQ_AFC), 7-Amino-4-trifluoromethylcoumarin (AFC), BugBuster® 10× Protein Extraction Reagent, Steriflip filters, and ACS grade hexane from Millipore Sigma; glycerol and lysozyme from VWR; deuterated chloroform from Cambridge Isotope Laboratories (99.8% D); cloning reagents from New England Biolabs; BL21 (DE3) pLysS competent cells from Novagen; pGEX-4T-1 GST vector from GenScript; 2.5-liter Ultra Yield Flasks from Thomson Instrument Company; antibiotics, media components, pre-made HEPES buffer (1 M pH 7.3), and Human Rhinovirus (HRV) 3C protease from Thermo Fisher; lysozyme from Thermo Scientific; imidazole from Teknova; 30-kDa Spin-X UF spin columns from Corning; HisTrap HP and HiTrap Q-HP columns from Cytiva; glycerol, bacterial protein extraction reagent II (B-PERII), and lysozyme from VWR; and (+)-α-bisabolol, (+)-epi-α-bisabolol, (−)-β-bisabolol and (−)-β-bisabolene from Toronto Research Chemicals. A vanillin-sulfuric acid solution was prepared by adding 7 g of vanillin and 1.3 mL of concentrated H2SO4 to 200 mL of methanol for TLC visualization. Certain bacterial strains were also used in the methods, such as E. coli. Chemically competent NEB Turbo cells were used for molecular cloning, chemically competent or electrocompetent S1030 cells (Addgene #105063) for luminescence studies and drop-based plating, DH5α for terpenoid production, and E. coli NEB BL21 (DE3) for protein overexpression.

Chemically competent cells were generated in six steps: (i) cells were plated on LB agar plates with the requisite antibiotics (listed in FIG. 84); (ii) individual colonies were used to inoculate 5 mL of LB media (25 g/L LB with antibiotics) in glass cultures tubes and grew these cultures overnight (37° C., 225 rpm); (iii) 100 μL of overnight culture were used to inoculate 10 mL of LB media (25 g/L LB with antibiotics) in 125-mL shake flasks, which we incubated for several hours (37° C., 225 rpm); (iv) when the OD600 reached 0.5-0.8, the cells were centrifuged (4000×g, 3 min, room temperature), removed the supernatant, and placed the pellets on ice; (v) the pellets were resuspended in 1 mL of ice-cold solution of 100 mM CaCl2 and 7% DMSO (sterile filtered); and (vi) cells were split into 100 μL aliquots and froze them at −80° C. for further use.

Electrocompetent cells were generated by following an approach similar to the one above. In step iv, the cells were resuspended in 1 mL of ice-cold Milli-Q water, then recentrifuged and resuspended in sterile ice-cold 20% glycerol twice. The pellets were frozen as before.

Luminescence assays were carried out in seven steps: (i) S1030 cells were transformed with protease-free B2H systems with and without pBad plasmids listed in FIG. 84; (ii) for each experiment, six colonies were used to inoculate six 1-mL cultures (terrific broth, or TB, at 2%, or 12 g/L tryptone, 24 g/L yeast extract, 12 mL/L 100% glycerol, 2.28 g/L KH2PO4, 12.53 g/L K2HPO4, pH=7.3, and antibiotics described in FIG. 84) in 96-well deep-well blocks, and the cultures grew overnight (37° C., 225 rpm); (iii) each overnight culture was diluted 1:100 in fresh TB (as in ii) with the following arabinose concentrations (% w/v): 0, 0.0002, 0.002, 0.02; (iv) each culture was incubated for 5.5 hours (37° C., 225 rpm); (v) 100 μL of each culture was added to a clear flat-bottom 96-well plate and measured both OD600 (absorbance at 600 nm) and luminescence (578 nm wavelength, 1000 ms integration, 1.0 mm read height) with a SpectraMax iD3 multi-mode plate reader (Molecular Devices).

The spectinomycin resistance of B2H-containing strains was examined through six steps: (i) S1030 cells were transformed with pIUP_FPP and variants of pTS and pB2H (Table S2), the transformed cells were plated on LB agar supplemented with antibiotics for plasmid maintenance (50 μg/ml kanamycin, 100 μg/ml carbenicillin, 10 μg/ml tetracycline, and 34 μg/ml chloramphenicol), and grown overnight (37° C.); (ii) single colonies were used to inoculate 1 mL TB (pH=7.0 supplemented with plasmid antibiotics) and grew the cells overnight (37° C. and 225 rpm); (iii) an aliquot of each culture was diluted 1:100 in TB (as above), and 3 μL of the dilution was plated on LB agar plates (pH=7.0) supplemented with 10 mM isoprenol, 50 μM IPTG, 20 mL/L glycerol, antibiotics for plasmid maintenance (as above), and varying concentrations of spectinomycin, unless otherwise specified in the figures; and (v) the cells were grown at 22° C. for at least 48-72 hours before photographing them.

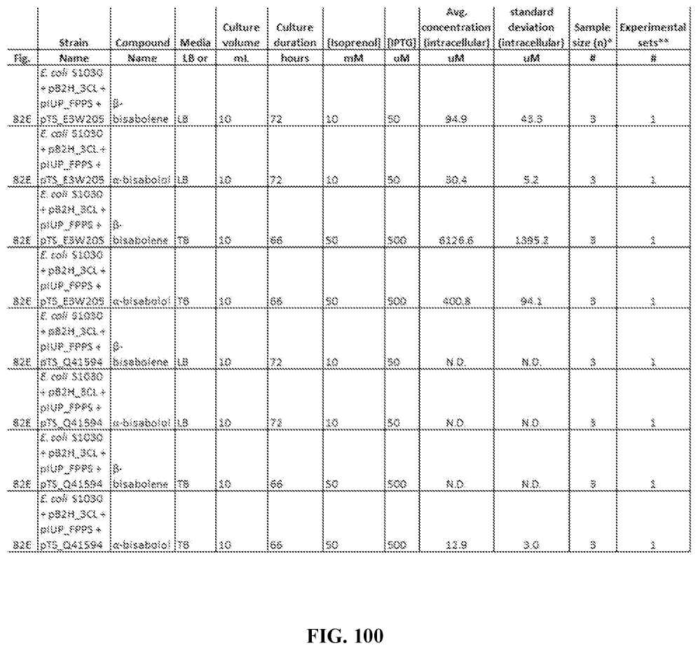

Small-scale terpenoid production was carried out in TB (pH=7.0) supplemented with antibiotics (FIG. 84). Briefly, S1030 cells harboring pIUP_FPPS and pTS were transformed, the cells were plated on LB agar, and grown overnight (37° C.). Colonies were used to inoculate 2 mL of TB, which were grown overnight (37° C., 225 rpm), and on the next morning, the culture was diluted with TB at a ratio of 1:75 in 10 mL TB and grown to an OD₆₀₀ of 0.3-0.6 (37° C., 225 rpm). The culture was induced by adding 10 mM isoprenol and 500 μM IPTG and grew it at 22° C. for 72 hours. FIG. 100 shows exact growth times for the cultures used to estimate intracellular titers (FIGS. 82A and 82E).

Terpenoids generated in liquid culture were measured with a gas chromatograph/mass spectrometer (GC-MS; a Trace 1310 GC fitted with a TG5-SilMS column, 15 m×0.25 mm, film thickness 0.25 μm and an ISQ 7000 MS; Thermo Fisher Scientific). All samples were prepared in hexane and diluted highly concentrated samples 10-20 times prior to bring concentrations within the MS detection limit. For full scans, the following GC method was used: hold at 40° C. (1 min), increase to 250° C. (30° C./min), hold at 250° C. (10 min). For the select-ion scans (SIM; FIG. 15), a 30 m column was used and the GC method was modified to hold at 80° C. (3 min), increase to 250° C. (15° C./min), hold at 250° C. (4 min), increase to 300° C. (60° C./min), hold at 300° C. (1 min). The m/z ratios were scanned from 50 to 350 and molecules were identified by using the NIST MS library. When necessary, this identification was confirmed with analytical standards or mass spectra reported in the literature. When quantifying terpenoids, m/z=204, a mass/charge ratio with a high signal for sesquiterpenes, was scanned.

To quantify α-bisabolol and β-bisabolene, GC/MS standard curves were built of structurally similar molecules. Store-bought (−)-α bisabolol was used, and, in the absence of a highly pure analytical standard of β-bisabolene, α-bisabolene isolated from bacterial cultures was used. A series of stocks of both standards in hexane was created and analyzed with GC/MS as outlined above.

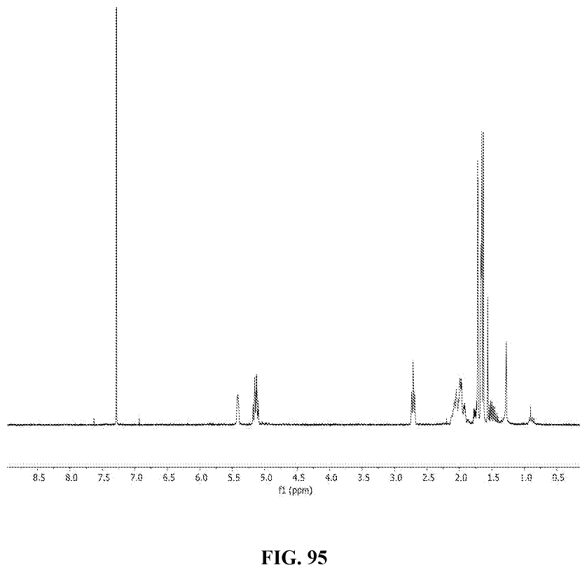

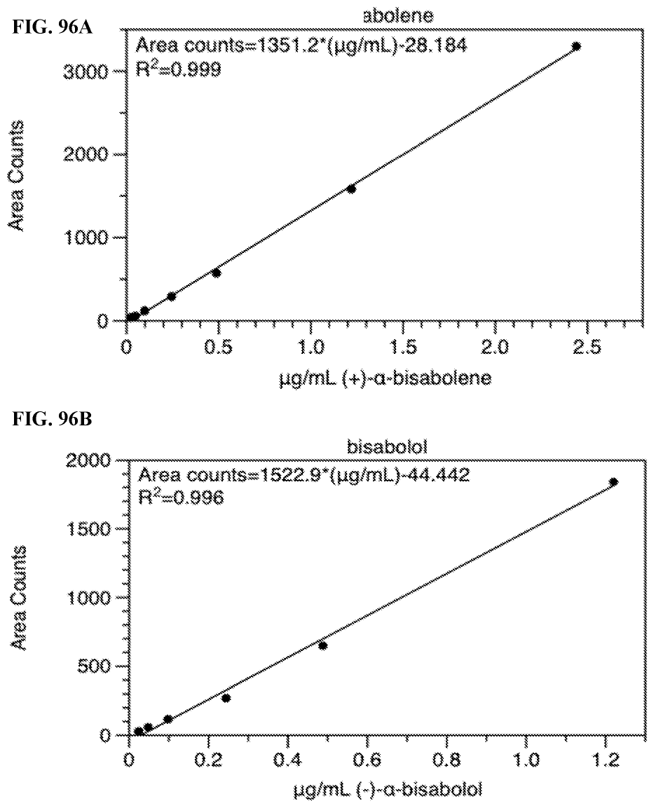

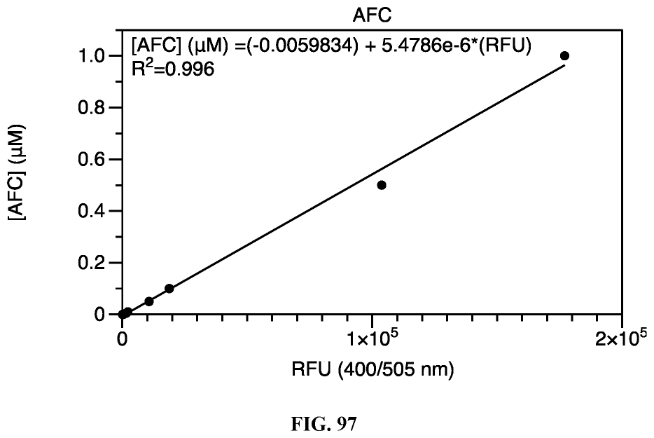

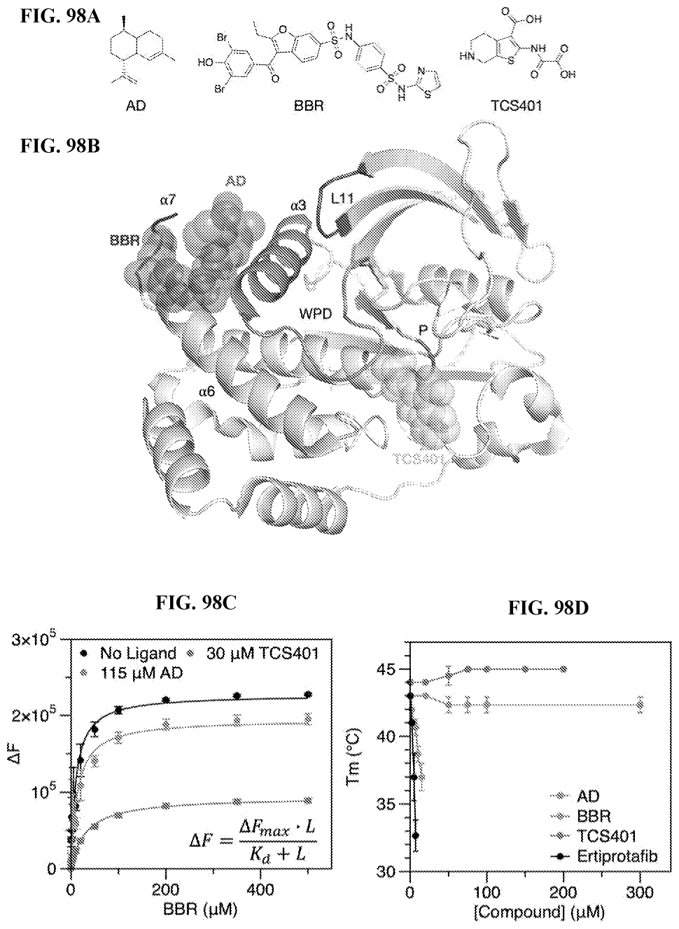

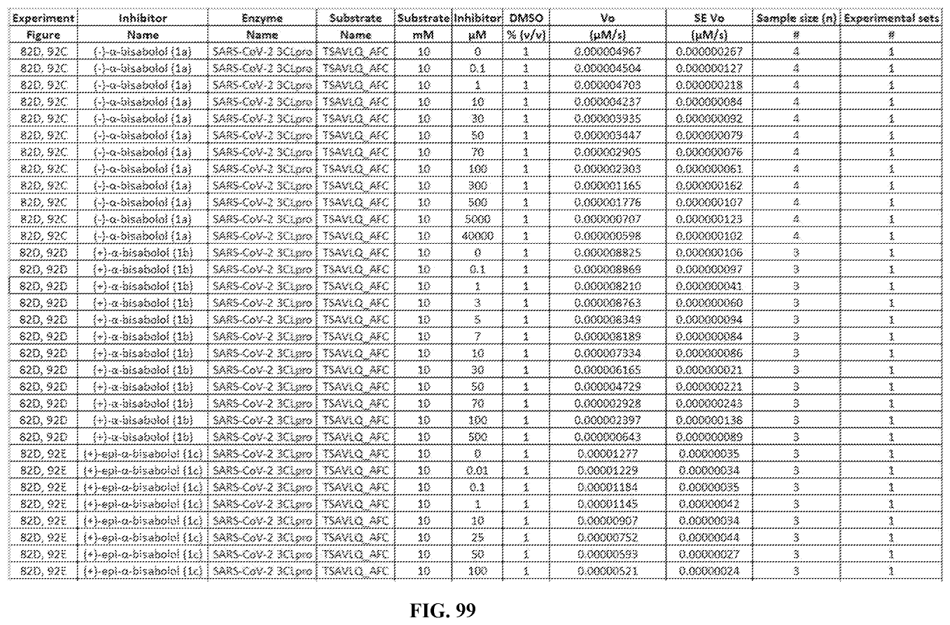

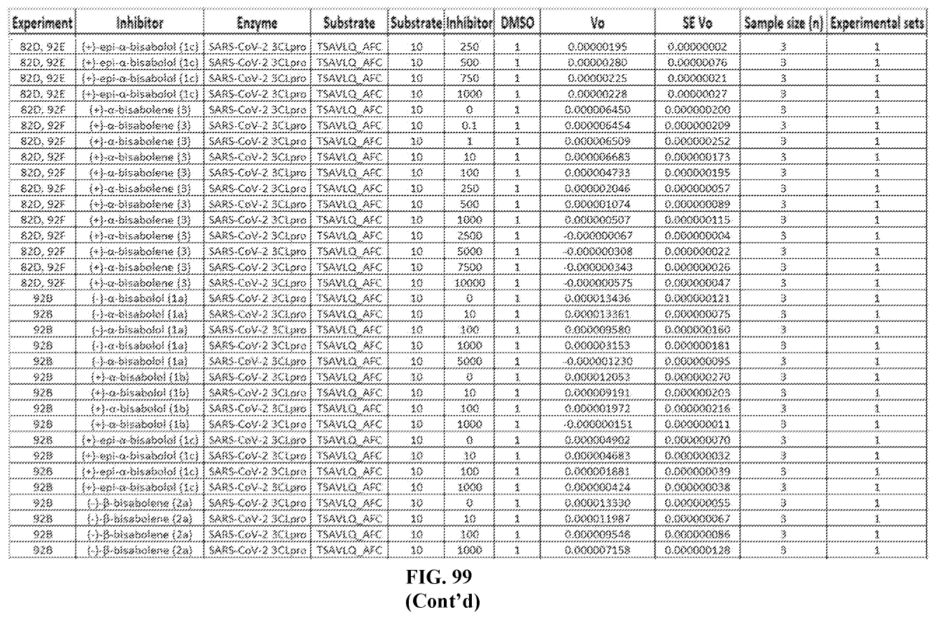

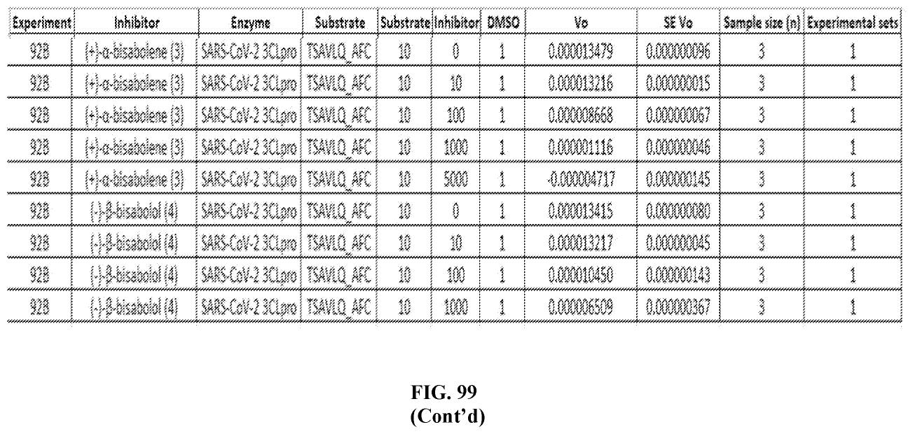

α-bisabolene was produced by carrying out the following steps: (i) E. coli DH5α was transformed with pAM45 and pTS containing α-bisabolene synthase (Uniprot ID: O81086) and used individual colonies to inoculate six 20-mL starter cultures (TB, pH-7.0 supplemented with plasmid antibiotics); (ii) each starter culture was used to inoculate a 50-mL culture (e.g., a 1:50 dilution in TB, pH=7.0), which was grown to an OD of 0.3-0.6 (37° C., 225 rpm), induced with 500 μM IPTG, and then grown for 144 hours (22° C., 225 rpm); (iii) the six 50-mL cultures were combined with 90 mL hexanes and agitated at room temperature for 30 minutes (vortexer); (iv) a separatory funnel was used to remove the hexanes and added them to a 500-mL centrifuge tube, which was spun at 4000 rpm for 20 minutes; (v) the supernatant was moved to a round bottom flask and evaporated the hexanes under vacuum to produce crude oil; (vi) 71.4 mg of crude oil was loaded onto a 5-g silica column (Sigma) and the non-α-bisabolene components were removed with vacuum liquid chromatography (VLC). This method yielded 25 5-ml fractions: 15 fractions with 0% ethyl acetate in hexanes, 5 fractions with 5% ethyl acetate in hexanes, and 5 fractions with 20% ethyl acetate in hexane; (vii) the fractions were analyzed with thin layer chromatography (TLC, 3:7 ethyl acetate/hexane) using vanillin acid-sulfuric acid as the detection method, where α-bisabolene appears as a dark blue spot on the TLC plates. Three fractions were enriched in α-bisabolene; (viii) these fractions were combined and dried with a rotary evaporator; (ix) the final composition was confirmed with ¹H NMR in CDCl3 (300 MHz, FIG. 95), with final spectrum being consistent with the published literature values. The combined fractions contained >95% pure α-bisabolene (1H NMR).

NMR spectroscopy was carried out at the BioFrontiers Nuclear Magnetic Resonance Facility at CU Boulder. All experiments were completed at 25° C. with a Bruker Accent 300 MHz spectrometer equipped with a Bruker 5 mm Smart Broadband Observe solution probe (BBFO), and final spectra were processed with MestReNova 14.2 software.

The stereochemistry of (−)-α-bisabolol, (+)-α-bisabolol, (+)-epi-α-bisabolol, (−)-β-bisabolol, and (−)-β-bisabolene reflect stereochemistry or specific rotation values reported in vendor certificates. The specific rotation of (+)-α-bisabolene was determined by using an Anton Paar MCP-200 polarimeter. In brief, the sodium D line was used at 589 nm with a cell path length of 100 mm. For α-bisabolene, 12.18 mg of the colorless oil was dissolved in 3.0 mL CHCl3 (0.406 g/100 mL CHCl3), placed the resulting solution inside the cell, and allowed the temperature to equilibrate to 25° C. before collecting a reading.

Intracellular concentrations of terpenoids were examined by extracting these compounds from cells grown in 4-mL cultures. Briefly, at 72 hours, 1 mL of cell culture was removed and centrifuged for 3 minutes (4000×g), and the supernatant was discarded. Terpenoids were extracted from the cell pellet by adding 600 μL hexane and 100-μL of 0.1-mm disrupter beads (Chemglass, CLS-1835-BG1) and vortexing the suspension for 30 minutes. The resulting lysate was centrifuged at 17,000×g for 10 minutes and analyzed the resulting hexane layer using GC/MS as described above. Finally, intracellular concentrations of each terpenoid (Ccell) were determined by using Eq. 1:

$${C}_{cell} = \frac{{C}_{culture} \cdot {V}_{hexane}}{η \cdot {OD}_{600} \cdot {C}_{OD} \cdot {V}_{cell}} ,$$

where C_{culture} is the concentration of terpenoids in the hexane, Vₕₑₓₐₙₑ is 600 μL, n is the extraction efficiency, C_{OD} is the OD-specific cell concentration (7.8×108 cells ml-1 OD-1), and V_{cell} is the volume of a single cell (4.4 fL/cell) 72. For initial estimates, η=1 was used, which assumes both complete cell lysis and complete partitioning of terpenoids from the aqueous to the organic layer; accordingly, the approach may underestimate intracellular terpenoid concentrations.

3CLpro was overexpressed in E. coli. In brief, BL21 (DE3) pLysS competent cells was transformed with a pGEX-4T-1 GST vector containing full-length 3CLpro with a 6× polyhistidine tag and a Human Rhinovirus (HRV) 3C protease site on its C-terminus (e.g., Q*GPHHHHHH (SEQ ID NO: 291), where Q is the C-terminal residue of the protein and * is the protease cleavage site). Two colonies were used to inoculate two 10-ml liquid cultures (LB supplemented with 50 μg/ml carbenicillin and 34 μg/ml chloramphenicol), which was grown overnight in an incubator shaker (37° C. and 200 rpm). These starter cultures were used to inoculate two one-liter cultures in 2.5-liter Ultra Yield Flasks, which were placed in an incubator shaker (37° C. and 200 rpm). At an OD600 of 0.65, the temperature was lowered to 16° C., protein expression was induced by adding 0.5 mM dioxane-free isopropyl-D-thiogalactopyranoside (IPTG), and grew the cultures for 18 hours. Final cultures were centrifuged, the pellets resuspended in 20 mL of Lysis Buffer (50 mM Tris, 1% Triton X-100, 300 mM NaCl, pH 8.0), and stored at −80° C.

3CLpro was purified from cell pellets by using fast protein liquid chromatography (FPLC). To begin, the frozen cell pellets were lysed by adding a solution containing 120 μl of Bond Breaker with 500 mM TCEP (Thermo Scientific), 100 μg lyophilized Lysozyme (Thermo Scientific), 2 mL BugBuster® 10× Protein Extraction Reagent (EMD Millipore), and 20 μl of 25 U/μl Benzonase (Millipore Sigma) to each pellet. These samples were rocked at room temperature for 1 hour and spun them down at 16000×g for 25 minutes. The supernatant from each lysis reaction was combined, with imidazole (Teknova) was added for a final concentration of 5 mM, and the final solution was filtered with 0.22 μm Steriflip filter (Millipore Sigma). Filtered solution was loaded onto a 5-mL HisTrap HP column (Cytiva) using a GE Akta Purifier 10, the column was washed with five column volumes of Tris buffer (50 mM Tris, 300 mM NaCl, 50 mM Imidazole, 0.5 mM TCEP, pH 8.0), and the protein was eluted with imidazole (50 mM to 200 mM imidazole). A 30-kDa Spin-X UF spin column (Corning) was used to concentrate the final protein to 10 mg/mL in cold HRV 3C cleavage buffer (50 mM Tris pH 7.0, 150 mM NaCl, 1 mM EDTA, 0.5 mM TCEP). Rhinovirus 3C Protease (Thermo Pierce) was added at a ratio of 1 mg HRV 3C for every 3 mg of 3CLpro and the proteolysis reaction was incubated at 4° C. for 16 hours. To remove the his-tagged HRV 3C protease and unproteolyzed 3CLpro, the final sample was diluted in Tris buffer (50 mM Tris, 300 mM NaCl, 0.5 mM TCEP, pH 8.0) to lower the imidazole concentration below 10 mM, loaded it onto 5-mL HisTrap HP column, and the flowthrough was collected. The final protein was filtered with a 0.45-μm filter, diluted 20-fold into Tris buffer (25 mM pH 8.0), and loaded onto an equilibrated 5-mL HiTrap Q-HP column (Cytiva). The loaded column was washed with five column volumes of Tris buffer (25 mM, pH 8.0) and eluted with salt (25 mM Tris, 500 mM NaCl, pH 8.0). The fractions were pooled with 3CLpro and exchanged into cold Tris buffer (50 mM Tris, 1 mM EDTA, 0.5 mM TCEP, pH 7.3), and the protein was concentrated to >10 mg/mL with a 30 kDa cutoff Spin-X UF spin column prior to freezing at −80° C.

The inhibitory effects of various compounds were characterized by measuring their influence on 3CLpro-catalyzed proteolysis of a fluorogenic peptide substrate (TSAVLQ_AFC). Briefly, 100-μL reactions were prepared consisting of 5 μg/mL SARS-COV-2 3CLpro, 10 μg/mL TSAVLQ_AFC, and 0.01-10,000 μM terpenoid in HEPES buffer (25 mM, pH=7.3) with 1% DMSO. These reactions were initiated by adding peptide substrate, and the proteolysis of fluorogenic peptide was monitored by measuring fluorescence (λex=400 nm, λem=505 nm) every 10 s for 10 min (SpectraMax iD3 plate reader).

While preferred embodiments of the present inventive concepts have been shown and described herein, it will be obvious to those skilled in the art that such embodiments are provided by way of example only. Numerous variations, changes, and substitutions will now occur to those skilled in the art without departing from the inventive concepts. It should be understood that various alternatives to the embodiments of the inventive concepts described herein may be employed in practicing the inventive concepts. It is intended that the following claims define the scope of the inventive concepts and that methods and structures within the scope of these claims and their equivalents be covered thereby.

TABLE 1
Gene Sources

| Component | Organism | Plasmid | Source |
|---|---|---|---|
| MevT_MBIS | Multiple | pAM45 | JBEI |

TABLE 2
Plasmids used in this study

| Plasmid | Description | Antibiotic* |
|---|---|---|
| F-plasmid | The F-plasmid from the S1030 strain of E. coli. | T |
| B2H | Bacterial two-hybrid system. Contains cI_SH2, | K |
| | RpoZ_S, c-Src, CDC37, PTP1B321, SpecR | |
| B2Hx | Bacterial two-hybrid system. Contains cI_SH2, | K |
| | RpoZ_S (Y/F mutation in substrate), c-Src, CDC37, | |
| | PTP1B321, SpecR | |
| pMBIS_{CmR} | A plasmid that harbors a mevalonate-dependent | P |
| | pathway for FPP production in E. coli and a | |
| | chloramphenicol resistance marker | |
| pAM45 | A plasmid that harbors the MevT and MBIS | P |
| | pathways (used for producing terenes for | |
| | purification) | |
| pTS_GHS | A plasmid that harbors GHS | C |
| pTS_GHS_{D343A} | A plasmid that harbors GHS (D343A mutation) | C |
| pTS_GHS_{A319Q} | A plasmid that harbors GHS (A319Q mutation) | C |
| pTS_GHS_{Y415C} | A plasmid that harbors GHS (Y415C mutation) | C |
| pTS_GHS_{A319Q, D343A} | A plasmid that harbors GHS (A319Q and | C |
| | inactivating D343A mutation) | |
| pTS_GHS_{Y415C, D343A} | A plasmid that harbors GHS (Y415C and | C |
| | inactivating D343A mutation) | |
| pTS_GHS_{S484L} | A plasmid that harbors GHS (S484L mutation) | C |
| pTS_GHS_{T455I} | A plasmid that harbors GHS (T455I mutation) | C |
| pTS_GHS_{L450T} | A plasmid that harbors GHS (L450T mutation) | C |
| pTS_GHS_{L450K} | A plasmid that harbors GHS (L450K mutation) | C |
| pTS_GHS_{S561C} | A plasmid that harbors GHS (S561C mutation) | C |
| pTS_GHS_{L450Y} | A plasmid that harbors GHS (L450Y mutation) | C |
| pTS_GHS_{L450G} | A plasmid that harbors GHS (L450G mutation) | C |
| pTS_GHS_{A319Q, Y415C} | A plasmid that harbors GHS (A319Q and Y415C | C |
| | mutations) | |
| pTS_GHS_{A319Q, Y415F} | A plasmid that harbors GHS (A319Q and Y415F | C |
| | mutations) | |
| pTS_GHS_{A319Q, S484A} | A plasmid that harbors GHS (A319Q and S484A | C |
| | mutations) | |
| pTS_GHS_{A319Q, S484G} | A plasmid that harbors GHS (A319Q and S484G | C |
| | mutations) | |
| pTS_GHS_{A319Q, L450I} | A plasmid that harbors GHS (A319Q and L450I | C |
| | mutations) | |
| pTS_GHS_{A319Q, Y415A} | A plasmid that harbors GHS (A319Q and Y415A | C |
| | mutations) | |
| pTS_GHS_{A319Q, A387T, V517L} | A plasmid that harbors GHS (A319Q, A387T, and | C |
| | V517L mutations) | |
| pTS_GHS_{A319Q, G459D} | A plasmid that harbors GHS (A319Q and G459D | C |
| | mutations) | |
| pTS_Empty | A plasmid with a pTrc promoter and no gene insert | C |
| pET21B_GHS | A plasmid with a T7 promoter and GHS including a | C |
| | C-terminal Hibit tag. | |
| pET21B_GHS_{A319Q} | A plasmid with a T7 promoter and GHS (A319Q | C |
| | mutation) including a C-terminal Hibit tag. | |

Antibiotic resistance: carbenicillin (C, 50 μg/ml), kanamycin (K, 50 μg/ml), tetracycline (T, 10 μg/ml), chloramphenicol (P, 34 μg/ml), and spectinomycin (S, conditional).
AG = Addgene accession # (Addgene.com).

TABLE 3
Primers used for mutagenesis

| | | | | SEQ |
|---|---|---|---|---|
| Mutant | F Primer | SEQ ID | R Primer | ID |
| GHS | CCCATGCGTGTCGTATA | 118 | CGATCTTGATGACAATGTTA | 119 |
| (D343A) | AGTCCGCTAACATTGTC | | GCGGACTTATACGACACGC | |
| | ATCAAGATCG | | ATGGG | |
| GHS | CCACTAAATTCTGGTTC | 120 | ATTTTACTTCTGGATGGCCG | 121 |
| (A319Q) | TGAAATCTGCGCGGCCA | | CGCAGATTTCAGAACCAGA | |
| | TCCAGAAGTAAAAT | | ATTTAGTGG | |
| GHS | GTTATACATAAACTGAA | 122 | CCACGTCCTGGCGCGGTGCA | 123 |
| (S561C) | TGCACCGCGCCAGGACG | | TTCAGTTTATGTATAAC | |
| | TGG | | | |
| GHS | CCTGCAAATACGCTTCC | 124 | AAAAACGCTTGGGAACGCT | 125 |
| (Y415C) | AGGCAGCGTTCCCAAGC | | GCCTGGAAGCGTATTTGCAG | |
| | GTTTTT | | G | |
| GHS | CAGCAACGGGATCAGAT | 126 | CAACACCGGTATGTGTGTAT | 127 |
| (L450Y) | TATATACACACATACCG | | ATAATCTGATCCCGTTGCTG | |
| | GTGTTG | | | |
| GHS | AGCAGCAACGGGATCA | 128 | CCAACACCGGTATGTGTGTA | 129 |
| (L450G) | GATTGCCTACACACATA | | GGCAATCTGATCCCGTTGCT | |
| | CCGGTGTTGG | | GCT | |
| GHS | AAGCAGCAACGGGATC | 130 | CCCAACACCGGTATGTGTGT | 131 |
| (L450K) | AGATTTTTTACACACAT | | AAAAAATCTGATCCCGTTGC | |
| | ACCGGTGTTGGG | | TGCTT | |
| GHS | AAGCAGCAACGGGATC | 132 | CCCAACACCGGTATGTGTGT | 133 |
| (L450T) | AGATTGGTTACACACAT | | AACCAATCTGATCCCGTTGC | |
| | ACCGGTGTTGGG | | TGCTT | |
| GHS | ATTAAGTACACACATAC | 134 | CTGAACAATGGCACCCCCA | 135 |
| (T445I) | CAATGTTGGGGGTGCCA | | ACATTGGTATGTGTGTACTT | |
| | TTGTTCAG | | AAT | |
| GHS | CGCATCATCGACCAGTC | 136 | CACCATCTGATTGAACTGGC | 137 |
| (S484L) | GCAGAGCCAGTTCAATC | | TCTGCGACTGGTCGATGATG | |
| | AGATGGTG | | CG | |

TABLE 4
Primers used for Gibson assembly of terpene
synthase hits

| Primer | Sequence | SEQ ID |
|---|---|---|
| Forward | AACAATTTCACACAGGAAACAGACC | 138 |
| Reverse | GCCTGCAGGTCGACTCTAGA | 139 |

TABLE 5
Scaling factor for longifolene/methyl abietate (m/z = 121)

| Technical | A_{std} | A_{ref} | C_{std} | C_{ref} | |
|---|---|---|---|---|---|
| Replicate | (counts*min) | (counts*min) | (μg/mL) | (μg/mL) | R |
| 1 | 2803042 | 5745500 | 20 | 6.12 | 0.15 |
| 2 | 2191084 | 4645592 | 20 | 6.12 | 0.14 |
| 3 | 1813136 | 3614250 | 20 | 6.12 | 0.15 |
| | | | | Avg R | 0.15 (0.004) |

TABLE 6
Gene Sources

| Component | Organism | Plasmid | Source |
|---|---|---|---|
| HIV-1Pr | Human immunodeficiency virus | Synthetic | Integrated DNA |
| | | | Technologies |
| 3CLpro | Severe acute respiratory | | |
| | syndrome coronavirus 2 | | |
| IUP | Multiple | pSEVA228-pro4IUPi | Addgene #122018 |
| GPPS | A. grandis | JBEI-15060 | Addgene #100962 |
| FPPS | E. coli | pMBIS | Addgene #17817 |
| GGPPS | T. Canadensis | pTS_TXS | Addgene #163839 |

TABLE 7
Plasmids

| Plasmid | Description | Antibiotic* |
|---|---|---|
| F-plasmid | The F-plasmid from the S1030 strain of E. coli. | T |
| pB2Hₙₒₙₑ | An early version of B2H that lacks a protease and a | K |
| | protease recognition sequence and includes LuxAB as the | |
| | GOI | |
| pB2H_{HIVcs} | A version of B2H that (i) lacks a protease, (ii) includes an | K |
| | HIV-1Pr recognition sequence, and (iii) contains LuxAB. | |
| pB2H_{2A-HIVcs} | A version of pB2H_{HIVcs} including two alanine residues | K |
| | flanking the recognition sequence | |
| pB2H_{4A-HIVcs} | A version of pB2H_{HIVcs} including four alanine residues | K |
| | flanking the recognition sequence | |
| pB2H_{3CLprocs} | A version of B2H that (i) lacks a protease, (ii) includes a | K |
| | 3CLpro recognition sequence, and (iii) contains LuxAB. | |
| pB2H_{2A-3CLprocs} | A version of pB2H_{3CLprocs} including two alanine residues | K |
| | flanking the recognition sequence | |
| pB2H_{4A-3CLprocs} | A version of pB2H_{HIVcs} including four alanine residues | K |
| | flanking the recognition sequence | |
| pBAD_{HIV-1Pr} | Arabinose-inducible expression of HIV-1Pr | P |
| pBAD_{HIV-1Pr} | Arabinose-inducible expression of 3ClPro | P |
| pB2H_{HIV}_{_{—}}_{10K} | A version of B2H that (i) includes HIV-1Pr with an | K |
| | engineered RBS (predicted TIR = 10,000) (ii) includes the | |
| | HIV-1Pr recognition sequence + three alanine residues, in | |
| | the MidT/RpoZ linker and (iii) contains SpecR. | |
| pB2H_{HIV}_{_{—}}_{10K}* | A version of B2H that (i) includes an inactive HIV-1Pr | K |
| | (D25N) with an engineered RBS (predicted TIR = 10,000) | |
| | (ii) includes the HIV-1Pr recognition sequence + three | |
| | alanine residues, in the MidT/RpoZ linker and (iii) | |
| | contains SpecR. | |
| pB2H_{HIV}_{_{—}}_{20K} | A version of B2H that (i) includes HIV-1Pr with an | K |
| | engineered RBS (predicted TIR = 20,000) (ii) includes the | |
| | HIV-1Pr recognition sequence + three alanine residues, in | |
| | the MidT/RpoZ linker and (iii) contains SpecR. | |
| pB2H_{HIV}_{_{—}}_{20K}* | A version of B2H that (i) includes an inactive HIV-1Pr | K |
| | (D25N) with an engineered RBS (predicted TIR = 20,000) | |
| | (ii) includes the HIV-1Pr recognition sequence + three | |
| | alanine residues, in the MidT/RpoZ linker and (iii) | |
| | contains SpecR. | |
| pB2H_{HIV}_{_{—}}_{100K} | A version of B2H that (i) includes HIV-1Pr with an | K |
| | engineered RBS (predicted TIR = 100,000) (ii) includes the | |
| | HIV-1Pr recognition sequence + three alanine residues, in | |
| | the MidT/RpoZ linker and (iii) contains SpecR. | |
| pB2H_{HIV}_{_{—}}_{100K}* | A version of B2H that (i) includes an inactive HIV-1Pr | K |
| | (D25N) with an engineered RBS (predicted TIR = 100,000) | |
| | (ii) includes the HIV-1Pr recognition sequence + three | |
| | alanine residues, in the MidT/RpoZ linker and (iii) | |
| | contains SpecR. | |
| pB2H_{3CLpro}_{_{—}}₁₀ | A version of B2H that (i) includes 3CLPro with an | K |
| | engineered RBS (predicted TIR = 10,000) (ii) includes the | |
| | 3CLpro recognition sequence with four additional alanine | |
| | residues flanking either side, in the MidT/RpoZ linker and | |
| | (iii) contains SpecR. | |
| pB2H_{3CLpro}_{_{—}}₁₀* | A version of B2H that (i) includes an inactive 3CLPro | K |
| | (H41A) with an engineered RBS (predicted TIR = 10,000) | |
| | (ii) includes the 3CLpro recognition sequence with four | |
| | additional alanine residues flanking either side, in the | |
| | MidT/RpoZ linker and (iii) contains SpecR. | |
| pB2H_{3CLpro}_{_{—}}₉₀ | A version of B2H that (i) includes 3CLPro with an | K |
| | engineered RBS (predicted TIR = 90,000) (ii) includes the | |
| | 3CLpro recognition sequence with four additional alanine | |
| | residues flanking either side, in the MidT/RpoZ linker and | |
| | (iii) contains SpecR. | |
| pB2H_{3CLpro}_{_{—}}₉₀* | A version of B2H that (i) includes an inactive 3CLPro | K |
| | (H41A) with an engineered RBS (predicted TIR = 90,000) | |
| | (ii) includes the 3CLpro recognition sequence with four | |
| | additional alanine residues flanking either side, in the | |
| | MidT/RpoZ linker and (iii) contains SpecR. | |
| pB2H_{3CLpro}_{_{—}}_{90, X} | A version of B2H that (i) includes an active 3CLPro | K |
| | (H41A) with an engineered RBS (ii) includes the 3CLpro | |
| | recognition sequence with four additional alanine residues | |
| | flanking either side, in the MidT/RpoZ linker (iii) includes | |
| | a Y/F mutation in the tyrosine substrate to inactive the | |
| | B2H, and (iv) contains SpecR. | |
| pIUP_{FPPS} | A plasmid that harbors ScCk, AtlPK, idi, and FPPS; all | P |
| | controlled by the LacUV5 promoter | |
| pIUP_{GGPPS} | A plasmid that harbors ScCk, AtlPK, idi, and GGPPS; all | P |
| | controlled by the LacUV5 promoter | |
| pB2Hopt | A version of B2H that (i) includes an active PTP1B | K |
| | catalytic domain and contains SpecR. | |
| pB2Hopt* | A version of B2H that (i) includes an inactive PTP1B | K |
| | catalytic domain (C215S) and contains SpecR. | |
| pB2Hopt₄₀₅ | A version of B2H that (i) includes an active PTP1B | K |
| | (residues 1-405) and contains SpecR. | |
| pB2Hopt₄₀₅* | A version of B2H that (i) includes an inactive PTP1B | K |
| | (C215S, residues 1-405) and contains SpecR. | |
| pB2H₂ | A version of B2H that (i) includes an active TCPTP | K |
| | catalytic domain and contains SpecR. | |
| pB2H₂* | A version of B2H that (i) includes an inactive TCPTP | K |
| | catalytic domain (R222M) and contains SpecR. | |
| pB2H_{2,387} | A version of B2H that (i) includes an active TCPTP | K |
| | (residues 1-387) and contains SpecR. | |
| pB2H_{2,387}* | A version of B2H that (i) includes an inactive TCPTP | K |
| | (R222M, residues 1-387) and contains SpecR. | |
| pB2H₁₂ | A version of B2H that (i) includes an active PEST | K |
| | catalytic domain (E57D) and contains SpecR. | |
| pB2H₁₂* | A version of B2H that (i) includes an inactive PEST | K |
| | catalytic domain (Y64A) and contains SpecR. | |
| pTS_A0A166A5J3 | A plasmid that harbors A0A166A5J3 | C |
| pTS_A0A0D9X487 | A plasmid that harbors A0A0D9X487 | C |
| pTS_F2DRF1 | A plasmid that harbors F2DRF1 | C |
| pTS_A2XI80 | A plasmid that harbors A2XI80 | C |
| pTS_A0A0D9ZGD1 | A plasmid that harbors A0A0D9ZGD1 | C |
| pTS_A0A0K9RZT8 | A plasmid that harbors A0A0K9RZT8 | C |
| pTS_A0A1I1AC30 | A plasmid that harbors A0A1I1AC30 | C |
| pTS_A0A1S3XW43 | A plasmid that harbors A0A1S3XW43 | C |
| pTS_A0A0D3D8G7 | A plasmid that harbors A0A0D3D8G7 | C |
| pTS_B9IF04 | A plasmid that harbors B9IF04 | C |
| pTS_A0A067L3D3 | A plasmid that harbors A0A067L3D3 | C |
| pTS_A0A0C2TFL3 | A plasmid that harbors A0A0C2TFL3 | C |
| pTS_A0A022S1C8 | A plasmid that harbors A0A022S1C8 | C |
| pTS_G4TNA6 | A plasmid that harbors G4TNA6 | C |
| pTS_A0A1L7WMZ8 | A plasmid that harbors A0A1L7WMZ8 | C |
| pTS_A0A078IZJ5 | A plasmid that harbors A0A078IZJ5 | C |
| pTS_A0A0C9VSL7 | A plasmid that harbors A0A0C9VSL7 | C |
| pTS_G2QRS0 | A plasmid that harbors G2QRS0 | C |
| pTS_A0A2H3DKU3 | A plasmid that harbors A0A2H3DKU3 | C |
| pTS_A0A0D2L718 | A plasmid that harbors A0A0D2L718 | C |
| pTS_T1LTV1 | A plasmid that harbors T1LTV1 | C |
| pTS_A0A287XU99 | A plasmid that harbors A0A287XU99 | C |
| pTS_A0A0G2ZSL3 | A plasmid that harbors A0A0G2ZSL3 | C |
| pTS_D2YZP9 | A plasmid that harbors D2YZP9 | C |
| pTS_E3W205 | A plasmid that harbors E3W205 | C |
| pTS_A0A3G9HBG7 | A plasmid that harbors A0A3G9HBG7 | C |
| pTS_Q41594 | A plasmid that harbors Q41594 | C |
| pTS_O64405 | A plasmid that harbors O64405 | C |
| pTS_UPI0018D1934E | A plasmid that harbors UPI0018D1934E | C |
| pTS_Q38710 | A plasmid that harbors Q38710 | C |
| pTS_Q9AR04 | A plasmid that harbors Q9AR04 | C |
| pTS_O81086 | A plasmid that harbors 081086 | C |
| pTS_Q49SP3 | A plasmid that harbors Q49SP3 | C |
| pTS_A0A2G9G5X4 | A plasmid that harbors A0A2G9G5X4 | C |
| pTS_Q40577 | A plasmid that harbors Q40577 | C |
| pTS_Q9XJ32 | A plasmid that harbors Q9XJ32 | C |
| pTS_G5CV46 | A plasmid that harbors G5CV46 | C |

Antibiotic resistance: carbenicillin (C, 50 μg/ml), kanamycin (K, 50 μg/ml), tetracycline (T, 10 μg/ml), chloramphenicol (P, 34 μg/ml), and spectinomycin (S, conditional).
AG = Addgene accession # (Addgene.com).

TABLE 8
Components of B2H systems.

| | | | SEQ | | SEQ |
|---|---|---|---|---|---|
| Component | Name | DNA | ID | Amino Acid | ID |
| HIV | HIV-1Pr | ATGGCGGATCGCC | 62 | MADRQGTVS | 63 |
| protease | | AGGGCACCGTGAG | | FNFPQITLWQ | |
| | | CTTTAACTTTCCGC | | RPLVTIKIGG | |
| | | AGATTACCCTGTG | | QLKEALLDTG | |
| | | GCAGCGCCCGCTG | | ADDTVLEEM | |
| | | GTGACCATTAAAA | | SLPGRWKPK | |
| | | TTGGCGGCCAGCT | | MIGGIGGFIK | |
| | | GAAAGAAGCGCTG | | VRQYDQILIEI | |
| | | CTGGATACCGGCG | | CGHKAIGTVL | |
| | | CGGATGATACCGT | | VGPTPVNIIG | |
| | | GCTGGAAGAAATG | | RNLLTQIGCT | |
| | | AGCCTGCCGGGCC | | LNF* | |
| | | GCTGGAAACCGAA | | | |
| | | AATGATTGGCGGC | | | |
| | | ATTGGCGGCTTTA | | | |
| | | TTAAAGTGCGCCA | | | |
| | | GTATGATCAGATT | | | |
| | | CTGATTGAAATTT | | | |
| | | GCGGCCATAAAGC | | | |
| | | GATTGGCACCGTG | | | |
| | | CTGGTGGGCCCGA | | | |
| | | CCCCGGTGAACAT | | | |
| | | TATTGGCCGCAAC | | | |
| | | CTGCTGACCCAGA | | | |
| | | TTGGCTGCACCCT | | | |
| | | GAACTTTTAA | | | |
| SARS CoV2 | 3CLpro | ATGTCGGGGTTCC | 68 | MSGFRKMAF | 69 |
| chymotrypsin- | | GTAAAATGGCTTT | | PSGKVEGCM | |
| like | | CCCCAGTGGCAAG | | VQVTCGTTTL | |
| protease | | GTAGAGGGATGTA | | NGLWLDDVV | |
| | | TGGTCCAAGTGAC | | YCPRHVICTS | |
| | | CTGTGGAACGACC | | EDMLNPNYE | |
| | | ACGTTAAATGGGT | | DLLIRKSNHN | |
| | | TGTGGCTTGATGA | | FLVQAGNVQ | |
| | | TGTAGTTTATTGTC | | LRVIGHSMQ | |
| | | CTCGCCACGTTATT | | NCVLKLKVD | |
| | | TGCACAAGTGAGG | | TANPKTPKY | |
| | | ATATGTTGAATCC | | KFVRIQPGQT | |
| | | TAATTATGAGGAT | | FSVLACYNGS | |
| | | CTGTTAATCCGTA | | PSGVYQCAM | |
| | | AATCGAATCATAA | | RPNFTIKGSFL | |
| | | TTTTCTTGTCCAAG | | NGSCGSVGF | |
| | | CGGGAAATGTTCA | | NIDYDCVSFC | |
| | | ATTGCGTGTTATC | | YMHHMELPT | |
| | | GGACACTCTATGC | | GVHAGTDLE | |
| | | AGAACTGCGTCCT | | GNFYGPFVD | |
| | | GAAGTTGAAAGTT | | RQTAQAAGT | |
| | | GATACGGCCAATC | | DTTITVNVLA | |
| | | CGAAGACGCCTAA | | WLYAAVING | |
| | | GTACAAGTTTGTG | | DRWFLNRFT | |
| | | CGCATTCAACCTG | | TTLNDFNLVA | |
| | | GACAGACATTTTC | | MKYNYEPLT | |
| | | TGTACTGGCGTGC | | QDHVDILGPL | |
| | | TACAACGGCAGCC | | SAQTGIAVLD | |
| | | CCAGCGGTGTATA | | MCASLKELL | |
| | | TCAGTGTGCAATG | | QNGMNGRTI | |
| | | CGCCCGAACTTTA | | LGSALLEDEF | |
| | | CAATCAAAGGGTC | | TPFDVVRQCS | |
| | | GTTTTTGAATGGT | | GVTFQ* | |
| | | AGTTGCGGCTCAG | | | |
| | | TTGGTTTCAACATT | | | |
| | | GATTATGATTGTG | | | |
| | | TCTCCTTTTGTTAC | | | |
| | | ATGCACCATATGG | | | |
| | | AGCTGCCAACCGG | | | |
| | | CGTGCATGCCGGC | | | |
| | | ACGGATTTGGAGG | | | |
| | | GCAATTTTTACGG | | | |
| | | ACCCTTTGTGGAC | | | |
| | | CGCCAAACAGCCC | | | |
| | | AGGCCGCAGGTAC | | | |
| | | TGATACCACTATC | | | |
| | | ACCGTCAACGTGC | | | |
| | | TTGCTTGGCTGTAC | | | |
| | | GCGGCGGTGATCA | | | |
| | | ATGGAGACCGCTG | | | |
| | | GTTCCTTAATCGTT | | | |
| | | TTACCACAACACT | | | |
| | | TAATGACTTCAAC | | | |
| | | TTGGTAGCAATGA | | | |
| | | AATACAACTACGA | | | |
| | | GCCTCTTACGCAG | | | |
| | | GACCATGTTGACA | | | |
| | | TCTTGGGTCCGCT | | | |
| | | GTCTGCACAGACT | | | |
| | | GGGATTGCTGTAC | | | |
| | | TTGATATGTGTGC | | | |
| | | AAGCTTAAAGGAA | | | |
| | | CTTCTTCAAAACG | | | |
| | | GTATGAATGGACG | | | |
| | | TACTATCCTTGGGT | | | |
| | | CGGCCTTATTAGA | | | |
| | | AGACGAGTTCACA | | | |
| | | CCGTTTGACGTTGT | | | |
| | | CCGCCAATGTAGC | | | |
| | | GGCGTAACTTTCC | | | |
| | | AATAA | | | |
| HIV-1Pr | HIV- | AAAGCTCGCGTAC | 110 | KARVLAEAM | 2 |
| cleavage | 1Prcs | TGGCCGAAGCCAT | | | |
| sequence | | G | | | |
| 3CLpro | 3CLprocs | GCAGTTTTACAAT | 109 | AVLQSGFR | 1 |
| cleavage | | CAGGGTTCCGT | | | |
| sequence | | | | | |
| RBS | 10kHIV | CTCAGAACTTTGC | 100 | N/A | |
| | | AAGGAGGTATTG | | | |
| RBS | 20kHIV | TATCCACAGTAAC | 101 | N/A | |
| | | ATAGGGGAGGATT | | | |
| | | AAT | | | |
| RBS | 100kHIV | AGGTAATTTATTT | 102 | N/A | |
| | | AAGATACACATAA | | | |
| | | GGAGGATATTAA | | | |
| RBS | 10K | TCGACAGCAGCCA | 103 | N/A | |
| | 3CLpro | ATAAGGAGGTATT | | | |
| | | A | | | |
| RBS | 90K | TCGACAGCAGCGG | 104 | N/A | |
| | 3CLpro | ATAAGGAGGTATT | | | |
| | | A | | | |

RBS designed computationally using the Ribosome Binding Site Calculator.

TABLE 9
Primers used for mutagenesis.

| Compo- | | SEQ | | SEQ |
|---|---|---|---|---|
| nent | F Primer | ID | R Primer | ID |
| HIVpr | AGCTGAAAGAAGCG | 140 | ACGGTATCATCCGCGCCGGT | 141 |
| D25N | CTGCTGAACACCGGC | | GTTCAGCAGCGCTTCTTTCA | |
| | GCGGATGATACCGT | | GCT | |
| 3CLpro | TATCCTCACTTGTGC | 142 | TGATGTAGTTTATTGTCCTC | 143 |
| H41A | AAATAACTGCGCGA | | GCGCAGTTATTTGCACAAGT | |
| | GGACAATAAACTAC | | GAGGATA | |
| | ATCA | | | |
| PTP1B | ACGGGCCCGTTGTGG | 144 | CAGACCTGCCGATGCCTGCA | 145 |
| C215S | TGCACAGCAGTGCA | | CTGCTGTGCACCACAACGGG | |
| | GGCATCGGCAGGTCT | | CCCGT | |
| | G | | | |
| TCPTP | CAGAGAGAAGGTGC | 146 | TGTAGTGCAGGCATTGGGAT | 147 |
| R222M | CAGACATCCCAATGC | | GTCTGGCACCTTCTCTCTG | |
| | CTGCACTACA | | | |
| PEST | GCTGTGATCAAATGG | 148 | GAAAAAGAAGAAAATGTTA | 149 |
| Y64A | CAGTATGTCCTTCGC | | AAAAGAACAGAGCGAAGGA | |
| | TCTGTTCTTTTTAAC | | CATACTGCCATTTGATCACA | |
| | ATTTTCTTCTTTTTC | | GC | |

TABLE 10
Terpene Synthases

| Component | Amino Acid | SEQ ID |
|---|---|---|
| FASTA Sequence: | MAQISESVSPSTDLKSTESSITSNRHGNMWEDDRIQ | 7 |
| >sp\|O64405\|TPSD5_ | SLNSPYGAPAYQERSEKLIEEIKLLFLSDMDDSCND | |
| ABIGR Gamma- | SDRDLIKRLEIVDTVECLGIDRHFQPEIKLALDYVYR | |
| humulene synthase | CWNERGIGEGSRDSLKKDLNATALGFRALRLHRYN | |
| OS = Abies grandis | VSSGVLENFRDDNGQFFCGSTVEEEGAEAYNKHVR | |
| OX = 46611 | CMLSLSRASNILFPGEKVMEEAKAFTTNYLKKVLA | |
| GN = ag5 PE = 1 | GREATHVDESLLGEVKYALEFPWHCSVQRWEARS | |
| SV = 1 | FIEIFGQIDSELKSNLSKKMLELAKLDFNILQCTHQK | |
| | ELQIISRWFADSSIASLNFYRKCYVEFYFWMAAAIS | |
| | EPEFSGSRVAFTKIAILMTMLDDLYDTHGTLDQLKI | |
| | FTEGVRRWDVSLVEGLPDFMKIAFEFWLKTSNELIA | |
| | EAVKAQGQDMAAYIRKNAWERYLEAYLQDAEWI | |
| | ATGHVPTFDEYLNNGTPNTGMCVLNLIPLLLMGEH | |
| | LPIDILEQIFLPSRFHHLIELASRLVDDARDFQAEKD | |
| | HGDLSCIECYLKDHPESTVEDALNHVNGLLGNCLL | |
| | EMNWKFLKKQDSVPLSCKKYSFHVLARSIQFMYN | |
| | QGDGFSISNKVIKDQVQKVLIVPVPI | |

TABLE 11
Viral Protease Targets for B2H Development.

| | | | Soluble | | Representative | |
|---|---|---|---|---|---|---|
| | Virus/Viral | | E. coli | Crystal | Substrate | SEQ |
| Family | Disease | Protease | Expression | Structure | Sequence | ID |
| Calciviridae | Norovirus | 3CLpro | Y | Y | FHLQ*GPED | 214 |
| | GI. 1 ^{B} | | | | | |
| | Norovirus | 3CLpro | Y | Y | FELQ*GPED | 215 |
| | GII. 4 ^{B} | | | | | |
| Coronaviridae | SARS-CoV ^{C, W} | 3CLpro | Y | Y | AVLQ*SGFR | 216 |
| | | PLpro | Y | Y | Deubiquitinase/ | |
| | | | | | deISGylase | |
| | MERS-CoV ^{C, W} | 3CLpro | Y | Y | GVLQ*SGLV | 217 |
| | | PLpro | Y | Y | Deubiquitinase/ | |
| | | | | | deISGylase | |
| Flaviviridae | Dengue | NS2B- | Y | Y | AGRR*SVSG | 218 |
| | Virus 1 ^{A} | NS3 | | | | |
| | Dengue | NS2B- | Y | Y | AGRK*SLTL | 219 |
| | Virus 2 ^{A} | NS3 | | | | |
| | Dengue | NS2B- | Y | Y | AGRK*SIAL | 220 |
| | Virus 3 ^{A} | NS3 | | | | |
| | Dengue | NS2B- | Y | Y | SGRK*SITL | 221 |
| | Virus 4 ^{A} | NS3 | | | | |
| | West Nile | NS2B- | Y | Y | SGKR*SQIG | 222 |
| | Virus ^{B} | NS3 | | | | |
| | Japanese | NS2B- | Y | Y | AGKR*SAVS | 223 |
| | encephalitis | NS3 | | | | |
| | virus ^{B} | | | | | |
| | St. Louis | NS2B- | Y | N | HSKR*GGAL | 224 |
| | encephalitis | NS3 | | | | |
| | virus ^{B} | | | | | |
| | Yellow | NS2B- | Y | Y | EGRR*GAAE | 225 |
| | fever | NS3 | | | | |
| | virus ^{B} | | | | | |
| | Zika | NS2B- | Y | Y | AGKR*GAAF | 226 |
| | Virus ^{B, W} | NS3 | | | | |
| Picornaviridae | Hepatitis | 3C | Y | Y | LRTQ*SFSN | 227 |
| | A ^{B} | protease | | | | |
| | Enterovirus | 3C | Y | Y | AKVQ*GPGF | 228 |
| | 68 ^{C} | protease | | | | |
| | Enterovirus | 3C | Y | Y | ATVQ*GPSL | 229 |
| | 71 ^{C} | protease | | | | |
| Poxviridase | Variola | K7L | Y | N | YTAG*NKVD | 230 |
| | Major | | | | | |
| | (smallpox) ^{A} | | | | | |
| | Monkeypox | I7L | N | N | YIAG/NKID | 231 |
| | virus ^{A} | | | | | |
| Nairoviridae | Crimean- | OTU | Y | Y | Deubiquitinase/ | |
| | Congo | domain | | | deISGylase | |
| | hemorrhagic | of L | | | | |
| | fever | protein | | | | |
| | orthonairo- | | | | | |
| | virus ^{A, W} | | | | | |
| Togaviridae | Venezuelanequine | NSP2 | Y | Y | EAGA*GSVE | 232 |
| | encephalitis | | | | | |
| | virus ^{B} | | | | | |
| | Eastern | NSP2 | N | N | EAGA*GSVE | 232 |
| | equine | | | | | |
| | encephalitis | | | | | |
| | virus ^{B} | | | | | |
| | Western | NSP2 | N | N | EAGA*GSVE | 232 |
| | equine | | | | | |
| | encephalitis | | | | | |
| | virus ^{B} | | | | | |
| | Chikungunya | NSP2 | Y | Y | RAGA*GIIE | 233 |
| | Virus ^{B} | | | | | |

^{A} denotes NIAID Priority Category A;
^{B} denotes NIAID Priority Category B;
^{C} denotes NIAID Priority Category C; and
^{W} denotes WHO Priority Emerging Infectious Disease.
*denotes the protease cleavage site.

TABLE 12
Sites selected for site-saturation mutagenesis (SSM). The accompanying
Excel file describes the highest scoring residues (see Eq.
1) and highlights the subset selected for SSM.

| GHS Site | EIS | Validating Data |
|---|---|---|
| S484 | A236 | Altered GHS and EIS profiles |
| S561 | W325 | Altered EIS profile |
| A319 | — | none |
| T445 | F198 | Altered GHS and EIS profiles |
| L450 | W203 | Altered EIS profile |
| Y415 | V168 | none |

TABLE 13
Gene Sources

| Component | Organism | Plasmid | Source |
|---|---|---|---|
| MevT_MBIS | Multiple | pAM45 | JBEI |

TABLE 14
Scaling factor for γ-humulene/methyl abietate (m/z = 121)

| Technical | A_{std} | A_{ref} | C_{std} | C_{ref} | |
|---|---|---|---|---|---|
| Replicate | (counts*min) | (counts*min) | (μg/mL) | (μg/mL) | R |
| 1 | 633426 | 212638 | 20 | 20.4 | 3.04 |
| 2 | 601844 | 199828 | 20 | 20.4 | 3.07 |
| 3 | 659373 | 659373 | 20 | 20.4 | 3.86 |
| | | | | Avg R | 3.3 (0.47) |

TABLE 15
Scaling factor for β-himachalene/methyl abietate (m/z = 121)

| Technical | A_{std} | A_{ref} | C_{std} | C_{ref} | |
|---|---|---|---|---|---|
| Replicate | (counts*min) | (counts*min) | (μg/mL) | (μg/mL) | R |
| 1 | 739567 | 4188031 | 20 | 100 | 0.88 |
| 2 | 662685 | 3574670 | 20 | 100 | 0.93 |
| 3 | 665655 | 3485161 | 20 | 100 | 0.95 |
| | | | | Avg R | 0.92 (0.034) |

TABLE 16
Scaling factor for himachalol/methyl abietate (m/z = 121)

| Technical | A_{std} | A_{ref} | C_{std} | C_{ref} | |
|---|---|---|---|---|---|
| Replicate | (counts*min) | (counts*min) | (μg/mL) | (μg/mL) | R |
| 1 | 546766 | 3495552 | 20 | 64 | 0.50 |
| 2 | 574092 | 3269965 | 20 | 64 | 0.54 |
| 3 | 545003 | 3354016 | 20 | 64 | 0.52 |
| | | | | Avg R | 0.52 (0.02) |

TABLE 17
Analysis of the inhibition of PTP1B₁₋₃₂₁ by himachalol.

| Model | SSE (μM²/s²) | DF | Criteria | Reference | Fit par. (μM) |
|---|---|---|---|---|---|
| Competitive | 0.0096 | 27 | Δᵢ = 28.0 | noncompetitive | Kᵢ = 24.4 |
| Uncompetitive | 0.0041 | 27 | Δᵢ = 5.12 | noncompetitive | Kᵢ = 252.8 |
| Noncompetitive* | 0.0034 | 27 | | | Kᵢ = 279.5* |
| Mixed | 0.0033 | 26 | F = 0.91 | noncompetitive | K_{i, c} = 160.7 |
| | | | p = 0.60 | | K_{i, u} = 305.4 |

TABLE 18
Details of hypothesis testing

| | Null | | | | | 95% confidence | P- |
|---|---|---|---|---|---|---|---|
| Figure | hypothesis | Δμ | Test | DF | t | intervals | value |
| 13B | Y415C − WT = 0 | 16.4 | One-tailed t- | 5.84 | 2.67 | (3.5, 29.3) | 0.02 |
| | | | test, unequal | | | | |
| | | | variance | | | | |
| 13B | A319Q/Y415A − | 37.6 | One-tailed t- | 5.89 | 39.8 | (34.8, 40.3) | 9.4*10⁻⁸ |
| | A319Q = 0 | | test, unequal | | | | |
| | | | variance | | | | |
| 13B | A319Q/Y415F − | 40.0 | One tailed t- | 2.39 | 17.0 | (30.2, 49.7) | 1.7*10⁻³ |
| | A319Q = 0 | | test, unequal | | | | |
| | | | variance | | | | |
| 13B | A319Q/Y415C − | 27.2 | One tailed t- | 2.21 | 7.0 | (14.3, 40.1) | 9.9*10⁻³ |
| | A319Q = 0 | | test, unequal | | | | |
| | | | variance | | | | |

TABLE 19
(S1). Gene Sources

| Component | Organism | Plasmid | Source |
|---|---|---|---|
| Src | H. sapiens | pB2Hopt | Addgene: 163830 |
| CDC37 | H. sapiens | pB2Hopt | Addgene: 163830 |
| LuxAB | P. luminescens | pB2Hopt | Addgene: 163830 |
| RpoZ | Escherichia coli | pB2Hopt | Addgene: 163830 |
| cI434 | Escherichia virus Lambda | pB2Hopt | Addgene: 163830 |
| SH2 | Rous sarcoma virus | pB2Hopt | Addgene: 78302 |
| SH2_{ABL} | H. sapiens | pB2H_{S1}_{_{—}}_{LuxAB} | Fox Lab: AKS1084 |
| MidT | Hamster polyoma virus | pB2Hopt | Addgene: 163830 |
| HA4 | H. sapiens | pB2H_{S1}_{_{—}}_{LuxAB} | Fox Lab: AKS1084 |
| IUP | S. cerevisiae, A. thaliana, | pSEVA228-pro4IUPi | Addgene: 122018 |
| | E. coli | | |
| FPPS | E. coli | pMBIS_CmR | Fox Lab: AKS1279 |
| Q9AR04 | Artemisia annua | pTS_Q9AR04 | Addgene: 19040 |
| O64405 | Abies grandis | pTS_O66405 | Addgene: 19003 |
| Q38710 | Abies grandis | pTS_Q38710 | Addgene: 163840 |
| Q41594 | Taxus brevifola | pTS_Q41594 | Addgene 163839 |
| O81086 | Abies grandis | pTS_O81086 | Addgene: 35153 |
| A0A166A5J3 | S. Suecicum HHB10207 ss-3 | pTS_A0A166A5J3 | Fox Lab¹³: AKS1513 |
| A0A0D9X487 | L. perrieri | pTS_A0A0D9X487 | Fox Lab: AKS1516 |
| F2DRF1 | H. vulgare | pTS_F2DRF1 | Fox Lab: AKS1519 |
| A2XI80 | O. sativa | pTS_A2XI80 | Fox Lab: AKS1522 |
| A0A0D9ZGD1 | O. glumipatula | pTS_A0A0D9ZGD1 | Fox Lab: AKS1525 |
| A0A0K9RZT8 | S. olaracea | pTS_A0A0K9RZT8 | Fox Lab: AKS1528 |
| A0A1I1AC30 | A. aquimarinus | pTS_A0A111AC30 | Fox Lab: AKS1531 |
| A0A1S3XW43 | N. tabacum | pTS_A0A1S3XW43 | Fox Lab: AKS1534 |
| A0A0D3D8G7 | B. oleracea | pTS_A0A0D3D8G7 | Fox Lab: AKS1514 |
| B9IF04 | P. trichocarpa | pTS_B9IF04 | Fox Lab: AKS1517 |
| A0A067L3D3 | J. curcas | pTS_A0A067L3D3 | Fox Lab: AKS1520 |
| A0A0C2TFL3 | A. Muscaria Koide BX008 | pTS_A0A0C2TFL3 | Fox Lab: AKS1523 |
| A0A022S1C8 | E. guttata | pTS_A0A022S1C8 | Fox Lab: AKS1537 |
| G4TNA6 | S. indica | pTS_G4TNA6 | Fox Lab: AKS1526 |
| A0A1L7WMZ8 | P. subalpine | pTS_A0A1L7WMZ8 | Fox Lab: AKS1529 |
| A0A078IZJ5 | B. napus | pTS_A0A078IZJ5 | Fox Lab: AKS1532 |
| A0A0C9VSL7 | S. stellatus SS14 | pTS_A0A0C9VSL7 | Fox Lab: AKS1535 |
| G2QRS0 | T. terrestris ATCC 38088 | pTS_G2QRS0 | Fox Lab: AKS1515 |
| A0A2H3DKU3 | A. gallica | pTS_A0A2H3DKU3 | Fox Lab: AKS1518 |
| A0A0D2L718 | H. sublateritium FD-334 | pTS_A0A0D2L718 | Fox Lab: AKS1521 |
| | SS-4 | | |
| T1LTV1 | T. urartu | pTS_T1LTV1 | Fox Lab: AKS1527 |
| A0A287XU99 | H. vulgare | pTS_A0A287XU99 | Fox Lab: AKS1530 |
| A0A0G2ZSL3 | A. gephyra | pTS_A0A0G2ZSL3 | Fox Lab: AKS1533 |
| Q49SP3 | P. cablin | pTS_Q49SP3 | Addgene: 108953 |
| D2YZP9 | Z. officinale | pTS_D2YZP9 | IDT DNA |
| E3W205 | S. austrocaledonicum | pTS_E3W205 | IDT DNA |
| B5GMG2 | S. clavuligerus | ptrc99a-GPPS-CSstr- | Addgene: 100962 |
| | | ispA*[JBEI-15060] | |
| A0A2G9G5X4 | H. impetigenosus | pTS_A0A2G9G5X4 | Twist Bioscience |
| A0A3G9HBG7 | A. maritima | pTS_A0A3G9HBG7 | Twist Bioscience |
| Q40577 | N. tabacum | pTS_Q40577 | Twist Bioscience |
| Q9XJ32 | S. tuberosum | pTS_Q9XJ32 | Twist Bioscience |
| G5CV46 | S. lycopersicum | pTS_G5CV46 | Twist Bioscience |
| 3CLpro | Severe acute respiratory | pBAD₂_{_{—}}_{3CL} | Twist Bioscience |
| (UNIPROT) | syndrome 2 (SARS-CoV-2) | | |
| HIVpro | Human immunodeficiency | pBAD₂_{_{—}}_{HIV} | IDT DNA |
| | virus (HIV) | | |
| PLpro | Severe acute respiratory | pBAD₂_{_{—}}_{PLpro} | Twist Bioscience |
| | syndrome 2 (SARS-CoV-2) | | |
| USP7 | H. sapiens | pBAD₂_{_{—}}_{USP7} | Twist Bioscience |
| 3CLpro | Severe acute respiratory | pB2H₂_{_{—}}_{0A}_{_{—}}_{3CL} | IDT DNA (primers |
| substrate | syndrome 2 (SARS-CoV-2) | | below) |
| HIVpro | Human immunodeficiency | pB2H₂_{_{—}}_{0A}_{_{—}}_{HIV} | IDT DNA (primers |
| substrate | virus | | below) |
| PLpro | Severe acute respiratory | pB2H₂_{_{—}}_{0A}_{_{—}}_{PL} | IDT DNA (primers |
| substrate | syndrome 2 (SARS-CoV-2) | | below) |
| USP7 | H. sapiens | pB2H₂_{_{—}}_{4A}_{_{—}}_{USP} | Twist Bioscience |
| substrate | | | |

TABLE 20
Plasmids.

| Plasmid | Description | Antibiotic* | Availability** |
|---|---|---|---|
| pB2H₂_{_{—}}_{None} | This bacterial two-hybrid (B2H) system | K | Fox Lab: AKS1254 |
| | links PTP1B inactivation to the | | |
| | expression of LuxAB. | | |
| pB2H_{S1}_{_{—}}_{LuxAB} | This B2H system links an SH2-HA4 | K | Fox Lab: AKS1084 |
| | interaction to the expression of LuxAB. | | |
| pB2H_{S1}_{_{—}}_{SpecR} | This B2H system links an SH2-HA4 | K | Fox Lab: pLK124 |
| | interaction to the expression of SpecR. | | |
| pB2H_{S1}_{_{—}}ₓ_{_{—}}_{LuxAB} | pB2H_{S1}_{_{—}}_{LuxAB} derivative with SH2 | K | Fox Lab: AKS1087 |
| | deletion. | | |
| pB2H_{S1}_{_{—}}ₓ_{_{—}}_{SpecR} | pB2H_{S1}_{_{—}}_{SpecR} derivative with SH2 | K | Fox Lab: pLK125 |
| | deletion. | | |
| pB2H₂_{_{—}}_{0A}_{_{—}}_{HIV} | pB2H₂_{_{—}}_{None} with KARVLAEAM added | K | Fox Lab: pLK7 |
| | to the linker of the substrate-RpoZ fusion | | |
| pB2H₂_{_{—}}_{2A}_{_{—}}_{HIV} | pB2H₂_{_{—}}_{None} with AAKARVLAEAMAA | K | Fox Lab: pLK18 |
| | added to the linker of the substrate-RpoZ | | |
| | fusion | | |
| pB2H₂_{_{—}}_{4A}_{_{—}}_{HIV} | pB2H₂_{_{—}}_{None} with | K | Fox Lab: pLK22 |
| | AAAAKARVLAEAMAAAA added to | | |
| | the linker of the substrate-RpoZ fusion | | |
| pB2H₂_{_{—}}_{0A}_{_{—}}_{3CL} | pB2H₂_{_{—}}_{None} with AVLQSGFR added to | K | Fox Lab: pLK8 |
| | the linker of the substrate-RpoZ fusion | | |
| pB2H₂_{_{—}}_{2A}_{_{—}}_{3CL} | pB2H₂_{_{—}}_{None} with AAAVLQSGFRAA | K | Fox Lab: pLK20 |
| | added to the linker of the substrate-RpoZ | | |
| | fusion | | |
| pB2H₂_{_{—}}_{4A}_{_{—}}_{3CL} | pB2H₂_{_{—}}_{None} with | K | Fox Lab: pLK23 |
| | AAAAAVLQSGFRAAAA added to the | | |
| | linker of the substrate-RpoZ fusion | | |
| pBAD₂_{_{—}}_{HIV} | A plasmid with HIVpro under control of | P | Fox Lab: AKS1123 |
| | the pBAD promoter | | |
| pBAD₂_{_{—}}_{HIVx} | A plasmid with catalytically inactive | P | Fox Lab: pLK31 |
| | HIVpro (or HIVpro D25N XXX) under | | |
| | control of the pBAD promoter | | |
| pBAD₂_{_{—}}_{3CL} | A plasmid with 3CLpro under control of | P | Fox Lab: pLK24 |
| | the pBAD promoter | | |
| pBAD₂_{_{—}}_{3CLx} | A plasmid with catalytically inactive | P | Fox Lab: pLK40 |
| | 3CLpro (or 3Clpro H41AXXX) under | | |
| | control of the pBAD | | |
| pBAD₂_{_{—}}_{PLpro} | A plasmid with PLpro under control of | P | Fox Lab: pLK39 |
| | the pBAD | | |
| pBAD₂_{_{—}}_{USP7} | A plasmid with an active USP7 chimeric | P | Fox Lab: pLK33 |
| | construct under control of the pBAD. | | |
| pBAD₂_{_{—}}_{USP7}_{_{—}}_{cat} | A plasmid with USP7 catalytic domain | P | Fox Lab: pLK97 |
| | under control of the pBAD | | |
| pB2H₂_{_{—}}_{0A}_{_{—}}_{PLP} | B2Hopt with LRGG added to the linker | K | Fox Lab: pLK44 |
| | of the substrate-RpoZ fusion | | |
| pB2H₂_{_{—}}_{4A}_{_{—}}_{PLP} | B2Hopt with AAAALRGGAAAA added | K | Fox Lab: pLK46 |
| | to the linker of the substrate-RpoZ fusion | | |
| pB2H₂_{_{—}}_{4A}_{_{—}}_{USP} | B2Hopt with AAAA_Ubiqutin_AAAA | K | Fox Lab: pLK85 |
| | added to the linker of the substrate-RpoZ | | |
| | fusion | | |
| pB2H2_{_{—}}_{PTP1B} | B2H opt from our prior study | K | AG: 163830 |
| pB2H2_{_{—}}_{PTP1Bx} | B2H opt with a catalytically inactive | K | AG: 163831 |
| | PTP1B (C215S) | | |
| pB2H2_{_{—}}_{3CL} | The B2H system for 3CLpro with | K | Fox Lab: AKS1719 |
| | AAAAAVLQS | | |
| | GFRAAAA added to the linker of the | | |
| | substrate-RpoZ fusion and 3CLpro in | | |
| | place of PTP1B. | | |
| pB2H2_{_{—}}_{3CLx} | pB2H2_3CL with 3CLpro (H41A) | K | Fox Lab: AKS1721 |
| pB2H2_{_{—}}_{HIV} | The B2H system for HIVpro with | K | Fox Lab: pLK48 |
| | KARVL | | |
| | AEAM added to the linker of the | | |
| | substrate-RpoZ fusion and HIVpro in | | |
| | place of PTP1B. | | |
| pB2H2_{_{—}}_{HIVx} | pB2H2_{_{—}}_{HIV} with HIVpro (D25N) | K | Fox Lab: pLK59 |
| pB2H2_{_{—}}_{PLP} | The B2H system for PLpro with | K | Fox Lab: pLK131 |
| | AAAA_Ubiquitin_LRG | | |
| | GAAAA added to the linker of the | | |
| | substrate-RpoZ fusion and PLpro in | | |
| | place of PTP1B. | | |
| pB2H2_{_{—}}_{PLPx} | pB2H2_{_{—}}_{PLP} with PLpro (C111S) | K | Fox Lab: pLK133 |
| F-plasmid | The F-plasmid from the S1030 strain of | T | AG 105063 |
| | E. coli. | | |
| pTS_{Q9AR04} | A plasmid that harbors Q9AR04 under | C | AG: 19040 |
| | control of the trc promoter. In the entries | | |
| | below, we refer to the base plasmid as | | |
| | pTS. | | |
| pTS_{O64405} | A plasmid that harbors O64405 | C | AG: 19003 |
| pTS_{O81086} | A plasmid that harbors O81086 | C | AG: 35153 |
| pTS_{A0A166A5J3} | A plasmid that harbors A0A166A5J3 | C | Fox Lab: AKS1513 |
| pTS_{A0A0D9X487} | A plasmid that harbors A0A0D9X487 | C | Fox Lab: AKS1516 |
| pTS_{F2DRF1} | A plasmid that harbors F2DRF1 | C | Fox Lab: AKS1519 |
| pTS_{A2XI80} | A plasmid that harbors A2XI80 | C | Fox Lab: AKS1522 |
| pTS_{A0A0D9ZGD1} | A plasmid that harbors A0A0D9ZGD1 | C | Fox Lab: AKS1525 |
| pTS_{A0A0K9RZT8} | A plasmid that harbors A0A0K9RZT8 | C | Fox Lab: AKS1528 |
| pTS_{A0A1I1AC30} | A plasmid that harbors A0A1I1AC30 | C | Fox Lab: AKS1531 |
| pTS_{A0A1S3XW43} | A plasmid that harbors A0A1S3XW43 | C | Fox Lab: AKS1534 |
| pTS_{A0A0D3D8G7} | A plasmid that harbors A0A0D3D8G7 | C | Fox Lab: AKS1514 |
| pTS_{B9IF04} | A plasmid that harbors B9IF04 | C | Fox Lab: AKS1517 |
| pTS_{A0A067L3D3} | A plasmid that harbors A0A067L3D3 | C | Fox Lab: AKS1520 |
| pTS_{A0A0C2TFL3} | A plasmid that harbors A0A0C2TFL3 | C | Fox Lab: AKS1523 |
| pTS_{A0A022S1C8} | A plasmid that harbors A0A022S1C8 | C | Fox Lab: AKS1537 |
| pTS_{G4TNA6} | A plasmid that harbors G4TNA6 | C | Fox Lab: AKS1526 |
| pTS_{A0A1L7WMZ8} | A plasmid that harbors A0A1L7WMZ8 | C | Fox Lab: AKS1529 |
| pTS_{A0A078IZJ5} | A plasmid that harbors A0A078IZJ5 | C | Fox Lab: AKS1532 |
| pTS_{A0A0C9VSL7} | A plasmid that harbors A0A0C9VSL7 | C | AG: 163841 |
| pTS_{G2QRS0} | A plasmid that harbors G2QRS0 | C | Fox Lab: AKS1515 |
| pTS_{A0A2H3DKU3} | A plasmid that harbors A0A2H3DKU3 | C | Fox Lab: AKS1518 |
| pTS_{A0A0D2L718} | A plasmid that harbors A0A0D2L718 | C | Fox Lab: AKS1521 |
| pTS_{T1LTV1} | A plasmid that harbors T1LTV1 | C | Fox Lab: AKS1527 |
| pTS_{A0A287XU99} | A plasmid that harbors A0A287XU99 | C | Fox Lab: AKS1530 |
| pTS_{A0A0G2ZSL3} | A plasmid that harbors A0A0G2ZSL3 | C | Fox Lab: AKS1533 |
| pTS_{G5CV46} | A plasmid that harbors G5CV46. | C | Fox Lab: AKS1672 |
| pTS_{Q9XJ32} | A plasmid that harbors Q9XJ32 | C | Fox Lab: AKS1671 |
| pTS_{Q40577} | A plasmid that harbors Q40577 | C | Fox Lab: AKS1673 |
| pTS_{A0A2G9G5X4} | A plasmid that harbors A0A2G95X4 | C | Fox Lab: AKS1670 |
| pTS_{Q49SP3} | A plasmid that harbors Q49SP3 | C | Fox Lab: AKS1422 |
| pTS_{A0A3G9HBG7} | A plasmid that harbors A0A3G9HBG7 | C | Fox Lab: AKS1650 |
| pTS_{D2YZP9} | A plasmid that harbors D2YZP9 | C | Fox Lab: AKS1430 |
| pTS_{E3W205} | A plasmid that harbors E3W205 | C | Fox Lab: AKS1431 |
| pTS_{Q41594} | A plasmid that harbors Q41594 | C | Fox Lab: EYK965 |
| pTS_{Q38710} | A plasmid that harbors Q38710 | C | Fox Lab: EYK949 |
| pTS_{B5GMG2} | A plasmid that harbors B5GMG2 | C | Fox Lab: AKS1655 |
| pTS_{empty} | A plasmid identical to all pTS plasmids, | C | Fox Lab: EYK953 |
| | but with no terpene synthase gene. | | |
| pIUP_FPPS | A plasmid that harbors the isoprenol- | P | Fox Lab: AKS1676 |
| | dependent terpenoid pathway and ispA, | | |
| | under control of the LacUV5 promoter. | | |
| pAM45 | A plasmid that harbors MevT, which | P | TB: pThink79 |
| | produces mevalonate, and MBIS. | | |

*Antibiotic resistance: carbenicillin (C, 50 μg/ml), kanamycin (K, 50 μg/ml), tetracycline (T, 10 μg/ml), chloramphenicol (P, 34 μg/ml), and spectinomycin (S, conditional).
**AG = Addgene accession # (Addgene.com). TB = Think Bioscience plasmid number.

TABLE 21
Components of Various B2H Systems

| | | | SEQ | | SEQ |
|---|---|---|---|---|---|
| | | | ID | Amino | ID |
| Component | Name | DNA | NO. | Acid | NO. |
| Kinase | c-Src | ATGGGCTCCAAG | 73 | MGSKPQTQ | 74 |
| | | CCGCAGACTCAG | | LAKDAWEI | |
| | | GGCCTGGCCAAG | | PRESLRLE | |
| | | GATGCCTGGGAG | | VKLGQGCF | |
| | | ATCCCTCGGGAG | | GEVWMGTW | |
| | | TCGCTGCGGCTG | | NGTTRVAI | |
| | | GAGGTCAAGCTG | | KTLKPGTM | |
| | | GGCCAGGGCTGC | | SPEAFLQE | |
| | | TTTGGCGAGGTG | | AQVMKKLR | |
| | | TGGATGGGGACC | | HEKLVQLY | |
| | | TGGAACGGTACC | | AVVSEEPI | |
| | | ACCAGGGTGGCC | | YIVTEYMS | |
| | | ATCAAAACCCTG | | KGSLLDFL | |
| | | AAGCCTGGCACG | | KGETGKYL | |
| | | ATGTCTCCAGAG | | RLPQLVDM | |
| | | GCCTTCCTGCAG | | AAQIASGM | |
| | | GAGGCCCAGGTC | | AYVERMNY | |
| | | ATGAAGAAGCTG | | VHRDLRAA | |
| | | AGGCATGAGAAG | | NILVGENL | |
| | | CTGGTGCAGTTG | | VCKVADFG | |
| | | TATGCTGTGGTT | | LARLIEDN | |
| | | TCAGAGGAGCCC | | EYTARQGA | |
| | | ATTTACATCGTC | | KFPIKWTA | |
| | | ACGGAGTACATG | | PEAALYGR | |
| | | AGCAAGGGGAGT | | FTIKSDVW | |
| | | TTGCTGGACTTT | | SFGILLTE | |
| | | CTCAAGGGGGAG | | LTTKGRVP | |
| | | ACAGGCAAGTAC | | YPGMVNRE | |
| | | CTGCGGCTGCCT | | VLDQVERG | |
| | | CAGCTGGTGGAC | | YRMPCPPE | |
| | | ATGGCTGCTCAG | | CPESLHDL | |
| | | ATCGCCTCAGGC | | MCQCWRKE | |
| | | ATGGCGTACGTG | | PEERPTFE | |
| | | GAGCGGATGAAC | | YLQAFLED | |
| | | TACGTCCACCGG | | YFTSTEPQ | |
| | | GACCTTCGTGCA | | YQPGENL | |
| | | GCCAACATCCTG | | | |
| | | GTGGGAGAGAAC | | | |
| | | CTGGTGTGCAAA | | | |
| | | GTGGCCGACTTT | | | |
| | | GGGCTGGCTCGG | | | |
| | | CTCATTGAAGAC | | | |
| | | AATGAGTACACG | | | |
| | | GCGCGGCAAGGT | | | |
| | | GCCAAATTCCCC | | | |
| | | ATCAAGTGGACG | | | |
| | | GCTCCAGAAGCT | | | |
| | | GCCCTCTATGGC | | | |
| | | CGCTTCACCATC | | | |
| | | AAGTCGGACGTG | | | |
| | | TGGTCCTTCGGG | | | |
| | | ATCCTGCTGACT | | | |
| | | GAGCTCACCACA | | | |
| | | AAGGGACGGGTG | | | |
| | | CCCTACCCTGGG | | | |
| | | ATGGTGAACCGC | | | |
| | | GAGGTGCTGGAC | | | |
| | | CAGGTGGAGCGG | | | |
| | | GGCTACCGGATG | | | |
| | | CCCTGCCCGCCG | | | |
| | | GAGTGTCCCGAG | | | |
| | | TCCCTGCACGAC | | | |
| | | CTCATGTGCCAG | | | |
| | | TGCTGGCGGAAG | | | |
| | | GAGCCTGAGGAG | | | |
| | | CGGCCCACCTTC | | | |
| | | GAGTACCTGCAG | | | |
| | | GCCTTCCTGGAG | | | |
| | | GACTACTTCACG | | | |
| | | TCCACCGAGCCC | | | |
| | | CAGTACCAGCCC | | | |
| | | GGGGAGAACCTC | | | |
| | | TAA | | | |
| Chaperone | CDC37 | ATGGTGGACTAC | 75 | MVDYSVWD | 76 |
| | | AGCGTGTGGGAC | | HIEVSDDE | |
| | | CACATTGAGGTG | | DETHPNID | |
| | | TCTGATGATGAA | | TASLFRWR | |
| | | GACGAGACGCAC | | HQARVERM | |
| | | CCCAACATCGAC | | EQFQKEKE | |
| | | ACGGCCAGTCTC | | ELDRGCRE | |
| | | TTCCGCTGGCGG | | CKRKVAEC | |
| | | CATCAGGCCCGG | | QRKLKELE | |
| | | GTGGAACGCATG | | VAEGGKAE | |
| | | GAGCAGTTCCAG | | LERLQAEA | |
| | | AAGGAGAAGGAG | | QQLRKEER | |
| | | GAACTGGACAGG | | SWEQKLEE | |
| | | GGCTGCCGCGAG | | MRKKEKSM | |
| | | TGCAAGCGCAAG | | PWNVDTLS | |
| | | GTGGCCGAGTGC | | KDGFSKSM | |
| | | CAGAGGAAACTG | | VNTKPEKT | |
| | | AAGGAGCTGGAG | | EEDSEEVR | |
| | | GTGGCCGAGGGC | | EQKHKTFV | |
| | | GGCAAGGCAGAG | | EKYEKQIK | |
| | | CTGGAGCGCCTG | | HFGMLRRW | |
| | | CAGGCCGAGGCA | | DDSQKYLS | |
| | | CAGCAGCTGCGC | | DNVHLVCE | |
| | | AAGGAGGAGCGG | | ETANYLVI | |
| | | AGCTGGGAGCAG | | WCIDLEVE | |
| | | AAGCTGGAGGAG | | EKCALMEQ | |
| | | ATGCGCAAGAAG | | VAHQTIVM | |
| | | GAGAAGAGCATG | | QFILELAK | |
| | | CCCTGGAACGTG | | SLKVDPRA | |
| | | GACACGCTCAGC | | CFRQFFTK | |
| | | AAAGACGGCTTC | | IKTADRQY | |
| | | AGCAAGAGCATG | | MEGFNDEL | |
| | | GTAAATACCAAG | | EAFKERVR | |
| | | CCCGAGAAGACG | | GRAKLRIE | |
| | | GAGGAGGACTCA | | KAMKEYEE | |
| | | GAGGAGGTGAGG | | EERKKRLG | |
| | | GAGCAGAAACAC | | PGGLDPVE | |
| | | AAGACCTTCGTG | | VYESLPEE | |
| | | GAAAAATACGAG | | LQKCFDVK | |
| | | AAACAGATCAAG | | DVQMLQDA | |
| | | CACTTTGGCATG | | ISKMDPTD | |
| | | CTTCGCCGCTGG | | AKYHMQRC | |
| | | GATGACAGCCAA | | IDSGLWVP | |
| | | AAGTACCTGTCA | | NSKASEAK | |
| | | GACAACGTCCAC | | EGEEAGPG | |
| | | CTGGTGTGCGAG | | DPLLEAVP | |
| | | GAGACAGCCAAT | | KTGDEKDV | |
| | | TACCTGGTCATT | | SV | |
| | | TGGTGCATTGAC | | | |
| | | CTAGAGGTGGAG | | | |
| | | GAGAAATGTGCA | | | |
| | | CTCATGGAGCAG | | | |
| | | GTGGCCCACCAG | | | |
| | | ACAATCGTCATG | | | |
| | | CAATTTATCCTG | | | |
| | | GAGCTGGCCAAG | | | |
| | | AGCCTAAAGGTG | | | |
| | | GACCCCCGGGCC | | | |
| | | TGCTTCCGGCAG | | | |
| | | TTCTTCACTAAG | | | |
| | | ATTAAGACAGCC | | | |
| | | GATCGCCAGTAC | | | |
| | | ATGGAGGGCTTC | | | |
| | | AACGACGAGCTG | | | |
| | | GAAGCCTTCAAG | | | |
| | | GAGCGTGTGCGG | | | |
| | | GGCCGTGCCAAG | | | |
| | | CTGCGCATCGAG | | | |
| | | AAGGCCATGAAG | | | |
| | | GAGTACGAGGAG | | | |
| | | GAGGAGCGCAAG | | | |
| | | AAGCGGCTCGGC | | | |
| | | CCCGGCGGCCTG | | | |
| | | GACCCCGTCGAG | | | |
| | | GTCTACGAGTCC | | | |
| | | CTCCCTGAGGAA | | | |
| | | CTCCAGAAGTGC | | | |
| | | TTCGATGTGAAG | | | |
| | | GACGTGCAGATG | | | |
| | | CTGCAGGACGCC | | | |
| | | ATCAGCAAGATG | | | |
| | | GACCCCACCGAC | | | |
| | | GCAAAGTACCAC | | | |
| | | ATGCAGCGCTGC | | | |
| | | ATTGACTCTGGC | | | |
| | | CTCTGGGTCCCC | | | |
| | | AACTCTAAGGCC | | | |
| | | AGCGAGGCCAAG | | | |
| | | GAGGGAGAGGAG | | | |
| | | GCAGGTCCTGGG | | | |
| | | GACCCATTACTG | | | |
| | | GAAGCTGTTCCC | | | |
| | | AAGACGGGCGAT | | | |
| | | GAGAAGGATGTC | | | |
| | | AGTGTGTAA | | | |
| Phosphatase | PTP1B | ATGGAGATGGAA | 5 | MEMEKEFE | 6 |
| | | AAGGAGTTCGAG | | QIDKSGSW | |
| | | CAGATCGACAAG | | AAIYQDIR | |
| | | TCCGGGAGCTGG | | HEASDFPC | |
| | | GCGGCCATTTAC | | RVAKLPKN | |
| | | CAGGATATCCGA | | KNRNRYRD | |
| | | CATGAAGCCAGT | | VSPFDHSR | |
| | | GACTTCCCATGT | | IKLHQEDN | |
| | | AGAGTGGCCAAG | | DYINASLI | |
| | | CTTCCTAAGAAC | | KMEEAQRS | |
| | | AAAAACCGAAAT | | YILTQGPL | |
| | | AGGTACAGAGAC | | PNTCGHFW | |
| | | GTCAGTCCCTTT | | EMVWEQKS | |
| | | GACCATAGTCGG | | RGVVMLNR | |
| | | ATTAAACTACAT | | VMEKGSLK | |
| | | CAAGAAGATAAT | | CAQYWPQK | |
| | | GACTATATCAAC | | EEKEMIFE | |
| | | GCTAGTTTGATA | | DTNLKLTL | |
| | | AAAATGGAAGAA | | ISEDIKSY | |
| | | GCCCAAAGGAGT | | YTVRQLEL | |
| | | TACATTCTTACC | | ENLTTQET | |
| | | CAGGGCCCTTTG | | REILHFHY | |
| | | CCTAACACATGC | | TTWPDFGV | |
| | | GGTCACTTTTGG | | PESPASFL | |
| | | GAGATGGTGTGG | | NFLFKVRE | |
| | | GAGCAGAAAAGC | | SGSLSPEH | |
| | | AGGGGTGTCGTC | | GPVVVHCS | |
| | | ATGCTCAACAGA | | AGIGRSGT | |
| | | GTGATGGAGAAA | | FCLADTCL | |
| | | GGTTCGTTAAAA | | LLMDKRKD | |
| | | TGCGCACAATAC | | PSSVDIKK | |
| | | TGGCCACAAAAA | | VLLEMRKF | |
| | | GAAGAAAAAGAG | | RMGLIQTA | |
| | | ATGATCTTTGAA | | DQLRFSYL | |
| | | GACACAAATTTG | | AVIEGAKF | |
| | | AAATTAACATTG | | IMGDSSVQ | |
| | | ATCTCTGAAGAT | | DQWKELSH | |
| | | ATCAAGTCATAT | | EDLEPPPE | |
| | | TATACAGTGCGA | | HIPPPPRP | |
| | | CAGCTAGAATTG | | PKRILEPH | |
| | | GAAAACCTTACA | | N | |
| | | ACCCAAGAAACT | | | |
| | | CGAGAGATCTTA | | | |
| | | CATTTCCACTAT | | | |
| | | ACCACATGGCCT | | | |
| | | GACTTTGGAGTC | | | |
| | | CCTGAATCACCA | | | |
| | | GCCTCATTCTTG | | | |
| | | AACTTTCTTTTC | | | |
| | | AAAGTCCGAGAG | | | |
| | | TCAGGGTCACTC | | | |
| | | AGCCCGGAGCAC | | | |
| | | GGGCCCGTTGTG | | | |
| | | GTGCACTGCAGT | | | |
| | | GCAGGCATCGGC | | | |
| | | AGGTCTGGAACC | | | |
| | | TTCTGTCTGGCT | | | |
| | | GATACCTGCCTC | | | |
| | | TTGCTGATGGAC | | | |
| | | AAGAGGAAAGAC | | | |
| | | CCTTCTTCCGTT | | | |
| | | GATATCAAGAAA | | | |
| | | GTGCTGTTAGAA | | | |
| | | ATGAGGAAGTTT | | | |
| | | CGGATGGGGCTG | | | |
| | | ATCCAGACAGCC | | | |
| | | GACCAGCTGCGC | | | |
| | | TTCTCCTACCTG | | | |
| | | GCTGTGATCGAA | | | |
| | | GGTGCCAAATTC | | | |
| | | ATCATGGGGGAC | | | |
| | | TCTTCCGTGCAG | | | |
| | | GATCAGTGGAAG | | | |
| | | GAGCTTTCCCAC | | | |
| | | GAGGACCTGGAG | | | |
| | | CCCCCACCCGAG | | | |
| | | CATATCCCCCCA | | | |
| | | CCTCCCCGGCCA | | | |
| | | CCCAAACGAATC | | | |
| | | CTGGAGCCACAC | | | |
| | | AATTGA | | | |
| Protease | 3CLpro | ATGTCGGGGTTC | 68 | MSGFRKMA | 69 |
| | | CGTAAAATGGCT | | FPSGKVEG | |
| | | TTCCCCAGTGGC | | CMVQVTCG | |
| | | AAGGTAGAGGGA | | TTTLNGLW | |
| | | TGTATGGTCCAA | | LDDVVYCP | |
| | | GTGACCTGTGGA | | RHVICTSE | |
| | | ACGACCACGTTA | | DMLNPNYE | |
| | | AATGGGTTGTGG | | DLLIRKSN | |
| | | CTTGATGATGTA | | HNFLVQAG | |
| | | GTTTATTGTCCT | | NVQLRVIG | |
| | | CGCCACGTTATT | | HSMQNCVL | |
| | | TGCACAAGTGAG | | KLKVDTAN | |
| | | GATATGTTGAAT | | PKTPKYKF | |
| | | CCTAATTATGAG | | VRIQPGQT | |
| | | GATCTGTTAATC | | FSVLACYN | |
| | | CGTAAATCGAAT | | GSPSGVYQ | |
| | | CATAATTTTCTT | | CAMRPNFT | |
| | | GTCCAAGCGGGA | | IKGSFLNG | |
| | | AATGTTCAATTG | | SCGSVGFN | |
| | | CGTGTTATCGGA | | IDYDCVSF | |
| | | CACTCTATGCAG | | CYMHHMEL | |
| | | AACTGCGTCCTG | | PTGVHAGT | |
| | | AAGTTGAAAGTT | | DLEGNFYG | |
| | | GATACGGCCAAT | | PFVDRQTA | |
| | | CCGAAGACGCCT | | QAAGTDTT | |
| | | AAGTACAAGTTT | | ITVNVLAW | |
| | | GTGCGCATTCAA | | LYAAVING | |
| | | CCTGGACAGACA | | DRWFLNRF | |
| | | TTTTCTGTACTG | | TTTLNDFN | |
| | | GCGTGCTACAAC | | LVAMKYNY | |
| | | GGCAGCCCCAGC | | EPLTQDHV | |
| | | GGTGTATATCAG | | DILGPLSA | |
| | | TGTGCAATGCGC | | QTGIAVLD | |
| | | CCGAACTTTACA | | MCASLKEL | |
| | | ATCAAAGGGTCG | | LQNGMNGR | |
| | | TTTTTGAATGGT | | TILGSALL | |
| | | AGTTGCGGCTCA | | EDEFTPFD | |
| | | GTTGGTTTCAAC | | VVRQCSGV | |
| | | ATTGATTATGAT | | TFQ | |
| | | TGTGTCTCCTTT | | | |
| | | TGTTACATGCAC | | | |
| | | CATATGGAGCTG | | | |
| | | CCAACCGGCGTG | | | |
| | | CATGCCGGCACG | | | |
| | | GATTTGGAGGGC | | | |
| | | AATTTTTACGGA | | | |
| | | CCCTTTGTGGAC | | | |
| | | CGCCAAACAGCC | | | |
| | | CAGGCCGCAGGT | | | |
| | | ACTGATACCACT | | | |
| | | ATCACCGTCAAC | | | |
| | | GTGCTTGCTTGG | | | |
| | | CTGTACGCGGCG | | | |
| | | GTGATCAATGGA | | | |
| | | GACCGCTGGTTC | | | |
| | | CTTAATCGTTTT | | | |
| | | ACCACAACACTT | | | |
| | | AATGACTTCAAC | | | |
| | | TTGGTAGCAATG | | | |
| | | AAATACAACTAC | | | |
| | | GAGCCTCTTACG | | | |
| | | CAGGACCATGTT | | | |
| | | GACATCTTGGGT | | | |
| | | CCGCTGTCTGCA | | | |
| | | CAGACTGGGATT | | | |
| | | GCTGTACTTGAT | | | |
| | | ATGTGTGCAAGC | | | |
| | | TTAAAGGAACTT | | | |
| | | CTTCAAAACGGT | | | |
| | | ATGAATGGACGT | | | |
| | | ACTATCCTTGGG | | | |
| | | TCGGCCTTATTA | | | |
| | | GAAGACGAGTTC | | | |
| | | ACACCGTTTGAC | | | |
| | | GTTGTCCGCCAA | | | |
| | | TGTAGCGGCGTA | | | |
| | | ACTTTCCAATAA | | | |
| Protease | HIVpro | ATGGCGGATCGC | 62 | MADRQGTV | 63 |
| | | CAGGGCACCGTG | | SFNFPQIT | |
| | | AGCTTTAACTTT | | LWQRPLVT | |
| | | CCGCAGATTACC | | IKIGGQLK | |
| | | CTGTGGCAGCGC | | EALLDTGA | |
| | | CCGCTGGTGACC | | DDTVLEEM | |
| | | ATTAAAATTGGC | | SLPGRWKP | |
| | | GGCCAGCTGAAA | | KMIGGIGG | |
| | | GAAGCGCTGCTG | | FIKVRQYD | |
| | | GATACCGGCGCG | | QILIEICG | |
| | | GATGATACCGTG | | HKAIGTVL | |
| | | CTGGAAGAAATG | | VGPTPVNI | |
| | | AGCCTGCCGGGC | | IGRNLLTQ | |
| | | CGCTGGAAACCG | | IGCTLNF* | |
| | | AAAATGATTGGC | | | |
| | | GGCATTGGCGGC | | | |
| | | TTTATTAAAGTG | | | |
| | | CGCCAGTATGAT | | | |
| | | CAGATTCTGATT | | | |
| | | GAAATTTGCGGC | | | |
| | | CATAAAGCGATT | | | |
| | | GGCACCGTGCTG | | | |
| | | GTGGGCCCGACC | | | |
| | | CCGGTGAACATT | | | |
| | | ATTGGCCGCAAC | | | |
| | | CTGCTGACCCAG | | | |
| | | ATTGGCTGCACC | | | |
| | | CTGAACTTTTAA | | | |
| Protease | PLpro | ATGGAGGTTCGT | 66 | MEVRTIKV | 67 |
| | | ACTATTAAGGTT | | FTTVDNIN | |
| | | TTTACCACAGTA | | LHTQVVDM | |
| | | GACAACATTAAT | | SMTYGQQF | |
| | | CTGCATACGCAG | | GPTYLDGA | |
| | | GTAGTAGATATG | | DVTKIKPH | |
| | | AGTATGACGTAC | | NSHEGKTF | |
| | | GGACAACAATTC | | YVLPNDDT | |
| | | GGGCCTACCTAC | | LRVEAFEY | |
| | | TTAGACGGAGCC | | YHTTDPSF | |
| | | GACGTAACGAAG | | LGRYMSAL | |
| | | ATTAAGCCACAC | | NHTKKWKY | |
| | | AATAGTCATGAG | | PQVNGLTS | |
| | | GGAAAGACATTT | | IKWADNNC | |
| | | TATGTCCTTCCT | | YLATALLT | |
| | | AATGACGACACT | | LQQIELKF | |
| | | CTGCGTGTAGAG | | NPPALQDA | |
| | | GCTTTCGAATAT | | YYRARAGE | |
| | | TACCACACGACC | | AANFCALI | |
| | | GACCCAAGTTTC | | LAYCNKTV | |
| | | TTGGGACGCTAT | | GELGDVRE | |
| | | ATGTCGGCCCTT | | TMSYLFQH | |
| | | AACCATACCAAG | | ANLDSCKR | |
| | | AAATGGAAGTAC | | VLNVVCKT | |
| | | CCGCAAGTCAAC | | CGQQQTTL | |
| | | GGGCTGACAAGC | | KGVEAVMY | |
| | | ATTAAATGGGCT | | MGTLSYEQ | |
| | | GATAATAATTGT | | FKKGVQIP | |
| | | TATCTGGCTACA | | CTCGKQAT | |
| | | GCATTATTAACA | | KYLVQQES | |
| | | TTGCAACAGATC | | PFVMMSAP | |
| | | GAACTTAAATTC | | PAQYELKH | |
| | | AATCCACCCGCT | | GTFTCASE | |
| | | CTTCAAGACGCC | | YTGNYQCG | |
| | | TACTACCGTGCC | | HYKHITSK | |
| | | CGCGCCGGTGAA | | ETLYCIDG | |
| | | GCAGCCAATTTC | | ALLTKSSE | |
| | | TGCGCTTTAATC | | YKGPITDV | |
| | | TTAGCTTATTGT | | FYKENSYT | |
| | | AATAAAACTGTT | | TTIK | |
| | | GGGGAACTTGGG | | | |
| | | GACGTACGTGAG | | | |
| | | ACGATGTCGTAC | | | |
| | | TTGTTTCAGCAT | | | |
| | | GCAAATCTGGAC | | | |
| | | TCGTGCAAACGT | | | |
| | | GTTCTGAACGTG | | | |
| | | GTGTGTAAGACG | | | |
| | | TGCGGACAGCAA | | | |
| | | CAAACTACTTTG | | | |
| | | AAGGGCGTCGAG | | | |
| | | GCTGTCATGTAT | | | |
| | | ATGGGCACGCTT | | | |
| | | AGCTACGAACAA | | | |
| | | TTTAAGAAGGGA | | | |
| | | GTTCAAATTCCT | | | |
| | | TGTACTTGCGGG | | | |
| | | AAGCAGGCAACA | | | |
| | | AAATATCTGGTT | | | |
| | | CAACAGGAAAGT | | | |
| | | CCGTTCGTTATG | | | |
| | | ATGTCTGCCCCA | | | |
| | | CCAGCACAATAC | | | |
| | | GAGCTTAAACAT | | | |
| | | GGAACCTTTACC | | | |
| | | TGCGCGAGTGAA | | | |
| | | TACACGGGAAAT | | | |
| | | TATCAATGTGGC | | | |
| | | CACTACAAGCAC | | | |
| | | ATTACGTCCAAG | | | |
| | | GAAACTTTATAC | | | |
| | | TGTATCGATGGT | | | |
| | | GCCCTGTTGACT | | | |
| | | AAGTCGTCGGAG | | | |
| | | TATAAAGGTCCG | | | |
| | | ATTACAGATGTA | | | |
| | | TTCTACAAAGAG | | | |
| | | AACTCTTACACC | | | |
| | | ACGACGATCAAG | | | |
| | | TAA | | | |
| Protease | USP7 | ATGAGTAAAAAG | 64 | MSKKHTGY | 65 |
| | | CATACAGGGTAC | | VGLKNQGA | |
| | | GTGGGCTTAAAA | | TCYMNSLL | |
| | | AACCAGGGCGCT | | QTLFFTNQ | |
| | | ACATGTTATATG | | LRKAVYMM | |
| | | AATTCGCTGTTA | | PTEGDDSS | |
| | | CAGACTTTATTT | | KSVPLALQ | |
| | | TTCACTAATCAG | | RVFYELQH | |
| | | TTACGTAAAGCG | | SDKPVGTK | |
| | | GTGTACATGATG | | KLTKSFGW | |
| | | CCTACGGAGGGG | | ETLDSFMQ | |
| | | GATGACTCGTCT | | HDVQELCR | |
| | | AAAAGCGTCCCG | | VLLDNVEN | |
| | | CTTGCCTTGCAA | | KMKGTCVE | |
| | | CGTGTCTTTTAC | | GTIPKLFR | |
| | | GAGTTGCAGCAC | | GKMVSYIQ | |
| | | TCGGACAAACCT | | CKEVDYRS | |
| | | GTAGGGACTAAA | | DRREDYYD | |
| | | AAGTTAACTAAA | | IQLSIKGK | |
| | | AGTTTTGGCTGG | | KNIFESFV | |
| | | GAAACTCTTGAC | | DYVAVEQL | |
| | | TCTTTCATGCAG | | DGDNKYDA | |
| | | CATGACGTTCAA | | GEHGLQEA | |
| | | GAACTTTGCCGT | | EKGVKFLT | |
| | | GTGCTGTTGGAC | | LPPVLHLQ | |
| | | AATGTAGAGAAC | | LMRFMYDP | |
| | | AAAATGAAAGGA | | QTDQNIKI | |
| | | ACATGCGTAGAA | | NDRFEFPE | |
| | | GGAACCATCCCG | | QLPLDEFL | |
| | | AAGTTGTTCCGC | | QKTDPKDP | |
| | | GGTAAAATGGTG | | ANYILHAV | |
| | | TCATATATTCAA | | LVHSGDNH | |
| | | TGTAAGGAAGTT | | GGHYVVYL | |
| | | GACTACCGCTCG | | NPKGDGKW | |
| | | GACCGTCGCGAA | | CKFDDDVV | |
| | | GATTACTATGAT | | SRCTKEEA | |
| | | ATCCAGCTGAGC | | IEHNYGGH | |
| | | ATTAAAGGGAAG | | DDDLSVRH | |
| | | AAAAACATTTTC | | CTNAYMLV | |
| | | GAGTCTTTCGTT | | YIRESKLS | |
| | | GATTACGTCGCG | | EVLQAVTD | |
| | | GTGGAGCAACTG | | HDIPQQLV | |
| | | GACGGAGATAAC | | ERLQEEKR | |
| | | AAGTATGACGCA | | IEAQKRKE | |
| | | GGGGAGCATGGT | | RQEGGGGG | |
| | | CTTCAAGAGGCC | | SGGGGGKA | |
| | | GAGAAGGGCGTT | | PKRSRYTY | |
| | | AAATTTTTAACA | | LEKAIKIH | |
| | | CTTCCCCCCGTC | | N | |
| | | CTGCATCTGCAG | | | |
| | | TTAATGCGCTTC | | | |
| | | ATGTACGATCCC | | | |
| | | CAGACGGATCAA | | | |
| | | AACATTAAAATC | | | |
| | | AACGATCGCTTT | | | |
| | | GAATTTCCAGAA | | | |
| | | CAGTTACCTTTG | | | |
| | | GACGAATTTTTG | | | |
| | | CAAAAAACAGAC | | | |
| | | CCAAAGGATCCG | | | |
| | | GCAAACTACATT | | | |
| | | TTACATGCAGTT | | | |
| | | TTAGTTCACTCT | | | |
| | | GGCGACAATCAC | | | |
| | | GGAGGGCACTAC | | | |
| | | GTTGTTTATTTA | | | |
| | | AACCCTAAAGGT | | | |
| | | GACGGTAAGTGG | | | |
| | | TGTAAGTTCGAC | | | |
| | | GACGACGTAGTC | | | |
| | | TCTCGTTGCACG | | | |
| | | AAGGAGGAGGCG | | | |
| | | ATTGAGCATAAT | | | |
| | | TATGGAGGGCAT | | | |
| | | GATGACGACCTT | | | |
| | | TCAGTTCGTCAT | | | |
| | | TGTACCAATGCG | | | |
| | | TACATGTTAGTG | | | |
| | | TATATCCGTGAA | | | |
| | | AGCAAGTTGTCA | | | |
| | | GAGGTACTGCAA | | | |
| | | GCTGTGACAGAT | | | |
| | | CATGACATCCCG | | | |
| | | CAACAACTGGTT | | | |
| | | GAACGCCTTCAG | | | |
| | | GAGGAAAAACGC | | | |
| | | ATCGAAGCACAG | | | |
| | | AAGCGCAAAGAA | | | |
| | | CGTCAAGAAGGT | | | |
| | | GGAGGAGGTGGT | | | |
| | | AGTGGAGGAGGA | | | |
| | | GGTGGGAAAGCG | | | |
| | | CCGAAGCGCAGC | | | |
| | | CGTTATACGTAC | | | |
| | | CTGGAAAAGGCT | | | |
| | | ATCAAAATTCAC | | | |
| | | AACTAA | | | |
| Protease | Non- | ATGGGTACAGAC | 77 | MGTDMWIE | 78 |
| | struct- | ATGTGGATCGAG | | RTADISWE | |
| | ural | CGTACCGCGGAC | | SDAEITGS | |
| | protein | ATTTCATGGGAA | | SERVDVRL | |
| | 2B-3 | AGTGACGCGGAG | | DDDGNFQL | |
| | protease | ATTACTGGCTCG | | MNDPGAGG | |
| | complex | TCAGAACGTGTT | | GGSGGGGG | |
| | from | GATGTTCGCCTT | | VLWDTPSP | |
| | West | GATGACGATGGC | | KEYKKGDT | |
| | Nile | AACTTTCAGTTG | | TTGVYRIM | |
| | Virus | ATGAACGACCCC | | TRGLLGSY | |
| | (WNV | GGTGCAGGGGGA | | QAGAGVMV | |
| | NS2B- | GGAGGCAGTGGC | | EGVFHTLW | |
| | NS3) | GGTGGGGGTGGG | | HTTKGAAL | |
| | | GTGTTGTGGGAT | | MSGEGRLD | |
| | | ACTCCGTCACCT | | PYWGSVKE | |
| | | AAAGAATACAAA | | DRLCYGGP | |
| | | AAAGGCGATACC | | WKLQHKWN | |
| | | ACTACTGGCGTG | | GQDEVQMI | |
| | | TACCGCATCATG | | VVEPGKNV | |
| | | ACCCGCGGGCTG | | KNVRTKPG | |
| | | CTTGGGTCATAT | | VFKTPEGE | |
| | | CAAGCCGGGGCA | | IGAVTLDF | |
| | | GGCGTAATGGTC | | PTGTSGSP | |
| | | GAGGGAGTATTT | | IVDKNGDV | |
| | | CATACTTTGTGG | | IGLYGNGV | |
| | | CACACGACTAAG | | IMPNGSYI | |
| | | GGGGCGGCTCTT | | SAIVQGKR | |
| | | ATGTCTGGCGAA | | MDEPIPAG | |
| | | GGTCGCTTAGAT | | FEPEMLGS | |
| | | CCGTATTGGGGA | | RS | |
| | | AGCGTCAAAGAG | | | |
| | | GATCGCTTATGC | | | |
| | | TACGGGGGACCT | | | |
| | | TGGAAATTACAA | | | |
| | | CATAAGTGGAAC | | | |
| | | GGTCAGGACGAA | | | |
| | | GTCCAAATGATC | | | |
| | | GTGGTTGAACCC | | | |
| | | GGCAAGAACGTG | | | |
| | | AAAAATGTTCGC | | | |
| | | ACAAAGCCTGGG | | | |
| | | GTGTTCAAGACG | | | |
| | | CCCGAGGGCGAA | | | |
| | | ATCGGTGCGGTG | | | |
| | | ACATTGGATTTC | | | |
| | | CCTACTGGCACC | | | |
| | | TCTGGATCACCA | | | |
| | | ATCGTTGATAAA | | | |
| | | AACGGCGATGTA | | | |
| | | ATCGGGTTATAC | | | |
| | | GGAAATGGGGTT | | | |
| | | ATTATGCCTAAT | | | |
| | | GGATCATATATC | | | |
| | | AGTGCCATTGTT | | | |
| | | CAGGGCAAACGT | | | |
| | | ATGGATGAGCCT | | | |
| | | ATCCCCGCCGGT | | | |
| | | TTCGAACCGGAA | | | |
| | | ATGTTAGGCTCA | | | |
| | | CGTTCTTAA | | | |
| Protease | Non- | ATGGGGTCGCAT | 150 | MGSHMLEA | 151 |
| | struct- | ATGCTTGAGGCG | | DLELERAA | |
| | ural | GACTTGGAGCTT | | DVRWEEQA | |
| | protein | GAACGCGCCGCT | | EISGSSPI | |
| | 2B-3 | GATGTACGTTGG | | LSITISED | |
| | protease | GAGGAGCAAGCA | | GSMSIKNE | |
| | complex | GAGATCAGCGGG | | EEEQTLGG | |
| | from | TCCTCTCCTATT | | GGSGGGGA | |
| | Dengue | CTGAGCATTACG | | GVLWDVPS | |
| | virus 2 | ATCAGCGAGGAT | | PPPVGKAE | |
| | (DENV2 | GGTTCAATGAGT | | LEDGAYRI | |
| | NS2B- | ATCAAGAATGAA | | KQKGILGY | |
| | NS3) | GAGGAGGAGCAA | | SQIGAGVY | |
| | | ACTTTGGGAGGC | | KEGTFHTM | |
| | | GGCGGATCGGGT | | WHVTRGAV | |
| | | GGGGGAGGCGCT | | LMHKGKRI | |
| | | GGTGTATTGTGG | | EPSWADVK | |
| | | GATGTGCCGTCA | | KDLISYGG | |
| | | CCCCCTCCGGTT | | GWKLEGEW | |
| | | GGAAAAGCGGAA | | KEGEEVQV | |
| | | CTTGAGGATGGC | | LALEPGKN | |
| | | GCTTATCGCATC | | PRAVQTKP | |
| | | AAGCAAAAAGGC | | GLFKTNTG | |
| | | ATCTTAGGATAC | | TIGAVSLD | |
| | | AGCCAAATTGGG | | FSPGTSGS | |
| | | GCTGGAGTATAC | | PIVDKKGK | |
| | | AAGGAGGGCACG | | VVGLYGNG | |
| | | TTCCATACGATG | | VVTRSGAY | |
| | | TGGCATGTTACC | | VSAIANTE | |
| | | CGTGGCGCGGTA | | KSIEDNPE | |
| | | TTAATGCACAAG | | IEDDIFRK | |
| | | GGAAAACGTATC | | | |
| | | GAGCCTTCGTGG | | | |
| | | GCAGATGTGAAA | | | |
| | | AAGGATTTGATT | | | |
| | | TCATATGGCGGT | | | |
| | | GGTTGGAAATTG | | | |
| | | GAGGGTGAATGG | | | |
| | | AAGGAAGGTGAG | | | |
| | | GAGGTTCAGGTT | | | |
| | | CTTGCCTTAGAA | | | |
| | | CCCGGAAAAAAC | | | |
| | | CCCCGCGCCGTT | | | |
| | | CAGACAAAACCT | | | |
| | | GGGTTATTTAAA | | | |
| | | ACTAACACTGGT | | | |
| | | ACGATTGGGGCC | | | |
| | | GTATCGTTAGAT | | | |
| | | TTCAGCCCTGGG | | | |
| | | ACTAGCGGTTCT | | | |
| | | CCGATCGTCGAC | | | |
| | | AAAAAGGGAAAA | | | |
| | | GTAGTGGGCTTA | | | |
| | | TATGGGAATGGG | | | |
| | | GTAGTGACTCGT | | | |
| | | TCTGGGGCATAC | | | |
| | | GTAAGCGCAATT | | | |
| | | GCAAACACTGAG | | | |
| | | AAATCGATTGAG | | | |
| | | GATAATCCTGAG | | | |
| | | ATCGAGGATGAC | | | |
| | | ATTTTCCGTAAG | | | |
| | | TAA | | | |
| Protease | PR_{3CLpro} | GCAGTTTTACAA | 109 | AVLQSGFR | 1 |
| recognition | | TCAGGGTTCCGT | | | |
| site | | | | | |
| Protease | PR_{HIVpro} | AAAGCTCGCGTA | 110 | KARVLAEA | 2 |
| recognition | | CTGGCCGAAGCC | | M | |
| site | | ATG | | | |
| Protease | PR_{PLpro} | TTACGTGGGGGG | 111 | LRGG | 3 |
| recognition | | | | | |
| site | | | | | |
| Protease | PR_{USP7} | CAAATCTTTGTC | 24 | QIFVKTLT | 25 |
| recognition | | AAGACATTAACA | | GKTITLEV | |
| site | | GGTAAGACCATC | | ESSDTIDN | |
| | | ACGTTGGAGGTA | | VKAKIQDK | |
| | | GAATCGAGTGAT | | EGIPPDQQ | |
| | | ACTATCGACAAT | | RLIFAGKQ | |
| | | GTAAAAGCAAAA | | LEDGRTLA | |
| | | ATCCAAGACAAG | | DYNIQKES | |
| | | GAGGGCATTCCC | | TLHLVLRL | |
| | | CCAGACCAGCAA | | RGG | |
| | | CGCTTGATTTTT | | | |
| | | GCGGGAAAGCAA | | | |
| | | CTTGAGGATGGC | | | |
| | | CGTACTTTAGCG | | | |
| | | GACTATAATATC | | | |
| | | CAGAAAGAATCT | | | |
| | | ACATTGCACTTA | | | |
| | | GTGTTGCGCCTG | | | |
| | | CGTGGGGGC | | | |
| Substrate | midT | GAACCGCAGTAT | 96 | EPQYEEIP | 97 |
| | | GAAGAAATTCCG | | IYL | |
| | | ATTTATCTG | | | |
| Substrate | midT | GAACCGCAGTTT | 98 | EPQFEEIP | 99 |
| | Y/F | GAAGAAATTCCG | | IYL | |
| | | ATTTATCTG | | | |
| Substrate | HA4 | GAACAAAAGCTT | 94 | EQKLISEE | 95 |
| | | ATTTCTGAAGAG | | DLGSSVSS | |
| | | GACTTGGGCAGC | | VPTKLEVV | |
| | | TCTGTGAGTAGC | | AATPTSLL | |
| | | GTTCCGACCAAA | | ISWDAPMS | |
| | | CTGGAAGTGGTT | | SSSVYYYR | |
| | | GCAGCAACCCCG | | ITYGETGG | |
| | | ACGAGCCTGCTG | | NSPVQEFT | |
| | | ATTTCTTGGGAT | | VPYSSSTA | |
| | | GCCCCGATGTCT | | TISGLSPG | |
| | | AGTAGCTCTGTG | | VDYTITVY | |
| | | TATTACTATCGT | | AWGEDSAG | |
| | | ATCACCTACGGT | | YMFMYSPI | |
| | | GAAACGGGCGGT | | SINYRTC | |
| | | AACAGCCCGGTG | | | |
| | | CAGGAATTTACG | | | |
| | | GTTCCGTATAGT | | | |
| | | AGCTCTACCGCG | | | |
| | | ACGATTAGTGGC | | | |
| | | CTGAGCCCGGGT | | | |
| | | GTGGATTACACC | | | |
| | | ATCACGGTTTAT | | | |
| | | GCATGGGGCGAA | | | |
| | | GATAGCGCGGGT | | | |
| | | TACATGTTCATG | | | |
| | | TATTCTCCGATT | | | |
| | | AGTATCAATTAC | | | |
| | | CGCACCTGC | | | |
| SH2 | SH2 | TGGTATTTTGGG | 90 | WYFGKITR | 91 |
| | | AAGATCACTCGT | | RESERLLL | |
| | | CGGGAGTCCGAG | | NPENPRGT | |
| | | CGGCTGCTGCTC | | FLVRESET | |
| | | AACCCCGAAAAC | | VKGAYALS | |
| | | CCCCGGGGAACC | | VSDFDNAK | |
| | | TTCTTGGTCCGG | | GLNVKHYL | |
| | | GAGAGCGAGACG | | IRKLDSGG | |
| | | GTAAAAGGTGCC | | FYITSRTQ | |
| | | TATGCCCTCTCC | | FSSLQQLV | |
| | | GTTTCTGACTTT | | AYYSKHAD | |
| | | GACAACGCCAAG | | GLCHRLTN | |
| | | GGGCTCAATGTG | | VC | |
| | | AAACACTACCTG | | | |
| | | ATCCGCAAGCTG | | | |
| | | GACAGCGGCGGC | | | |
| | | TTCTACATCACC | | | |
| | | TCACGCACACAG | | | |
| | | TTCAGCAGCCTG | | | |
| | | CAGCAGCTGGTG | | | |
| | | GCCTACTACTCC | | | |
| | | AAACATGCTGAT | | | |
| | | GGCTTGTGCCAC | | | |
| | | CGCCTGACCAAC | | | |
| | | GTCTGC | | | |
| SH2 | SH2_{ABL} | AGTCTGGAAAAA | 92 | SLEKHSWY | 93 |
| | | CACAGCTGGTAT | | HGPVSRNA | |
| | | CATGGCCCTGTG | | AEYLLSSG | |
| | | AGCCGTAACGCG | | INGSFLVR | |
| | | GCCGAATACCTG | | ESESSPGQ | |
| | | CTGAGCTCTGGC | | RSISLRYE | |
| | | ATTAATGGTTCT | | GRVYHYRI | |
| | | TTTCTGGTTCGT | | NTASDGKL | |
| | | GAAAGTGAAAGT | | YVSSESRF | |
| | | AGCCCGGGCCAG | | NTLAELVH | |
| | | CGCAGCATTTCT | | HHSTVADG | |
| | | CTGCGTTATGAA | | LITTLHYP | |
| | | GGTCGCGTGTAT | | APKR | |
| | | CACTACCGTATC | | | |
| | | AACACCGCCAGC | | | |
| | | GATGGCAAACTG | | | |
| | | TACGTTTCTAGT | | | |
| | | GAATCTCGCTTC | | | |
| | | AATACCCTGGCA | | | |
| | | GAACTGGTGCAT | | | |
| | | CACCATAGCACG | | | |
| | | GTTGCGGATGGT | | | |
| | | CTGATCACCACG | | | |
| | | CTGCATTATCCG | | | |
| | | GCGCCGAAACGC | | | |
| Promoter | pBAD | AGAAACCAATTG | 82 | N/A | |
| | | TCCATATTGCAT | | | |
| | | CAGACATTGCCG | | | |
| | | TCACTGCGTCTT | | | |
| | | TTACTGGCTCTT | | | |
| | | CTCGCTAACCAA | | | |
| | | ACCGGTAACCCC | | | |
| | | GCTTATTAAAAG | | | |
| | | CATTCTGTAACA | | | |
| | | AAGCGGGACCAA | | | |
| | | AGCCATGACAAA | | | |
| | | AACGCGTAACAA | | | |
| | | AAGTGTCTATAA | | | |
| | | TCACGGCAGAAA | | | |
| | | AGTCCACATTGA | | | |
| | | TTATTTGCACGG | | | |
| | | CGTCACACTTTG | | | |
| | | CTATGCCATAGC | | | |
| | | ATTTTTATCCAT | | | |
| | | AAGATTAGCG | | | |
| Promoter | Pro1¹⁴ | TTCTAGAGCACA | 83 | N/A | |
| | | GCTAACACCACG | | | |
| | | TCGTCCCTATCT | | | |
| | | GCTGCCCTAGGT | | | |
| | | CTATGAGTGGTT | | | |
| | | GCTGGATAACTT | | | |
| | | TACGGGCATGCA | | | |
| | | TAAGGCTCGGTA | | | |
| | | TCTATATTCAGG | | | |
| | | GAGACCACAACG | | | |
| | | GTTTCCCTCTAC | | | |
| | | AAATAATTTTGT | | | |
| | | TTAACTTTTACT | | | |
| | | AGAG | | | |
| Promoter | plac- | CATTAGGCACCC | 84 | N/A | |
| | Zopt¹⁵ | CGGGCTTTACTC | | | |
| | | GTAAAGCTTCCG | | | |
| | | GCGCGTATGTTG | | | |
| | | TGTCGACCG | | | |
| Promoter | ProD¹⁶ | TTCTAGAGCACA | 85 | N/A | |
| | | GCTAACACCACG | | | |
| | | TCGTCCCTATCT | | | |
| | | GCTGCCCTAGGT | | | |
| | | CTATGAGTGGTT | | | |
| | | GCTGGATAACTT | | | |
| | | TACGGGCATGCA | | | |
| | | TAAGGCTCGGTA | | | |
| | | TCTATATTCAGG | | | |
| | | GAGACCACAACG | | | |
| | | GTTTCCCTCTAC | | | |
| | | AAATAATTTTGT | | | |
| | | TTAACTTTTACT | | | |
| | | AGAG | | | |
| RBS_{3CL} | TIR_90K | TCGACAGCAGCG | 104 | N/A | |
| | | GATAAGGAGGTA | | | |
| | | TTA | | | |
| RBS_{HIV} | TIR_20K | TATCCACAGTAA | 101 | N/A | |
| | | CATAGGGGAGGA | | | |
| | | TTAAT | | | |
| RBS_{USP} | TIR_80 | ACAATTCATATC | 105 | N/A | |
| | | CTAAGCGCTTCT | | | |
| | | TAA | | | |
| RBS_{PL} | TIR_10K | ATTTGCCACCAT | 106 | N/A | |
| | | TAAAGGAGGTTC | | | |
| | | CAA | | | |
| RBS_{T7opt} | GOI | GTGCAGTAAGGA | 107 | N/A | |
| (Pre- | | GGAAAAAAAA | | | |
| optimized) | | | | | |
| RBS_{T7opt} | GOI_{L2-93} | GGGCCGACTTGC | 108 | N/A | |
| (optimized) | | GGTATAATAA | | | |
| cI | cI N- | ATGAGTATCAGC | 86 | MSISSRVK | 87 |
| | terminal | AGCAGGGTAAAA | | SKRIQLGL | |
| | domain | AGCAAAAGAATT | | NQAELAQK | |
| | | CAGCTTGGACTT | | VGTTQQSI | |
| | | AACCAGGCTGAA | | EQLENGKT | |
| | | CTTGCTCAAAAG | | KRPRFLPE | |
| | | GTGGGGACTACC | | LASALGVS | |
| | | CAGCAGTCTATA | | VDWLLNGT | |
| | | GAGCAGCTCGAA | | SDSNVRFV | |
| | | AACGGTAAAACT | | GHVEPKGK | |
| | | AAGCGACCACGC | | YPLISMVR | |
| | | TTTTTACCAGAA | | ARSWCEAC | |
| | | CTTGCGTCAGCT | | EPYDIKDI | |
| | | CTTGGCGTAAGT | | DEWYDSDV | |
| | | GTTGACTGGCTG | | NLLGNGFW | |
| | | CTCAATGGCACC | | LKVEGDSM | |
| | | TCTGATTCGAAT | | TSPVGQSI | |
| | | GTTAGATTTGTT | | PEGHMVLV | |
| | | GGGCACGTTGAG | | DTGREPVN | |
| | | CCCAAAGGGAAA | | GSLVVAKL | |
| | | TATCCATTGATT | | TDANEATF | |
| | | AGCATGGTTAGA | | KKLVIDGG | |
| | | GCTCGTTCGTGG | | QKYLKGLN | |
| | | TGTGAAGCTTGT | | PSWPMTPI | |
| | | GAACCCTACGAT | | NGNCKIIG | |
| | | ATCAAGGACATT | | VVVEARVK | |
| | | GATGAATGGTAT | | FVD | |
| | | GACAGTGACGTT | | | |
| | | AACTTATTAGGC | | | |
| | | AATGGATTCTGG | | | |
| | | CTGAAGGTTGAA | | | |
| | | GGTGATTCCATG | | | |
| | | ACCTCACCTGTA | | | |
| | | GGTCAAAGCATC | | | |
| | | CCTGAAGGTCAT | | | |
| | | ATGGTGTTAGTA | | | |
| | | GATACTGGACGG | | | |
| | | GAGCCAGTGAAT | | | |
| | | GGAAGCCTTGTT | | | |
| | | GTAGCCAAACTG | | | |
| | | ACTGACGCGAAC | | | |
| | | GAAGCAACATTC | | | |
| | | AAGAAACTGGTC | | | |
| | | ATAGATGGCGGT | | | |
| | | CAGAAGTACCTG | | | |
| | | AAAGGCCTGAAT | | | |
| | | CCTTCATGGCCT | | | |
| | | ATGACTCCTATC | | | |
| | | AACGGAAACTGC | | | |
| | | AAGATTATCGGT | | | |
| | | GTTGTCGTGGAA | | | |
| | | GCGAGGGTAAAA | | | |
| | | TTCGTAGAC | | | |
| cI operator | cI | acaagaaagttt | 113 | | |
| | operator | gt | | | |
| | 2 (OR2) | | | | |
| cI operator | cI | acaagatacatt | 114 | | |
| | operator | gt | | | |
| | 3 (OR3) | | | | |
| CymR DNA | CymR | ATGAGCCCGAAA | 152 | MSPKRRTQ | 153 |
| Binding | | CGTCGTACCCAG | | AERAMETQ | |
| Protein | | GCAGAACGTGCA | | GKLIAAAL | |
| | | ATGGAAACCCAG | | GVLREKGY | |
| | | GGTAAACTGATT | | AGFRIADV | |
| | | GCAGCAGCACTG | | PGAAGVSR | |
| | | GGTGTTCTGCGT | | GAQSHHFP | |
| | | GAAAAAGGTTAT | | TKLELLLA | |
| | | GCAGGTTTTCGT | | TFEWLYEQ | |
| | | ATTGCAGATGTT | | ITERSRAR | |
| | | CCGGGTGCAGCC | | LAKLKPED | |
| | | GGTGTTAGCCGT | | DVIQQMLD | |
| | | GGTGCACAGAGC | | DAAEFFLD | |
| | | CATCATTTTCCG | | DDFSIGLD | |
| | | ACCAAACTGGAA | | LIVAADRD | |
| | | CTGCTGCTGGCA | | PALREGIQ | |
| | | ACCTTTGAATGG | | RTVERNRF | |
| | | CTGTATGAGCAG | | VVEDMWLG | |
| | | ATTACCGAACGT | | VLVSRGLS | |
| | | AGCCGTGCACGT | | RDDAEDIL | |
| | | CTGGCAAAACTG | | WLIFNSVR | |
| | | AAACCGGAAGAT | | GLVVRSLW | |
| | | GATGTTATTCAG | | QKDKERFE | |
| | | CAGATGCTGGAT | | RVRNSTLE | |
| | | GATGCAGCAGAA | | IARERYAK | |
| | | TTTTTTCTGGAT | | FKR | |
| | | GATGATTTTAGC | | | |
| | | ATCGGCCTGGAT | | | |
| | | CTGATTGTTGCA | | | |
| | | GCAGATCGTGAT | | | |
| | | CCGGCACTGCGT | | | |
| | | GAAGGTATTCAG | | | |
| | | CGTACCGTTGAA | | | |
| | | CGTAATCGTTTT | | | |
| | | GTTGTTGAAGAT | | | |
| | | ATGTGGCTGGGT | | | |
| | | GTGCTGGTGAGC | | | |
| | | CGTGGTCTGAGC | | | |
| | | CGTGATGATGCC | | | |
| | | GAAGATATTCTG | | | |
| | | TGGCTGATTTTT | | | |
| | | AACAGCGTTCGT | | | |
| | | GGTCTGGTAGTT | | | |
| | | CGTAGCCTGTGG | | | |
| | | CAGAAAGATAAA | | | |
| | | GAACGTTTTGAA | | | |
| | | CGTGTGCGTAAT | | | |
| | | AGCACCCTGGAA | | | |
| | | ATTGCACGTGAA | | | |
| | | CGTTATGCAAAA | | | |
| | | TTCAAACGT | | | |
| CymR | CuO | aacaaacagaca | 115 | N/A | |
| operator | | atctggtctgtt | | | |
| | | tgta | | | |
| Ph1F DNA | Ph1F | ATGGCACGTACC | 154 | MARTPSRS | 155 |
| binding | | CCGAGCCGTAGC | | SIGSLRSP | |
| protein | | AGCATTGGTAGC | | HTHKAILT | |
| | | CTGCGTAGTCCG | | STIEILKE | |
| | | CATACCCATAAA | | CGYSGLSI | |
| | | GCAATTCTGACC | | ESVARRAG | |
| | | AGCACCATTGAA | | AGKPTIYR | |
| | | ATCCTGAAAGAA | | WWTNKAAL | |
| | | TGTGGTTATAGC | | IAEVYENE | |
| | | GGTCTGAGCATT | | IEQVRKFP | |
| | | GAAAGCGTGGCA | | DLGSFKAD | |
| | | CGTCGCGCCGGT | | LDFLLHNL | |
| | | GCAGGCAAACCG | | WKVWRETI | |
| | | ACCATTTATCGT | | CGEAFRCV | |
| | | TGGTGGACCAAC | | IAEAQLDP | |
| | | AAAGCAGCACTG | | VTLTQLKD | |
| | | ATTGCCGAAGTG | | QFMERRRE | |
| | | TATGAAAATGAA | | IPKKLVED | |
| | | ATCGAACAGGTA | | AISNGELP | |
| | | CGTAAATTTCCG | | KDINRELL | |
| | | GATTTGGGTAGC | | LDMIFGFC | |
| | | TTTAAAGCCGAT | | WYRLLTEQ | |
| | | CTGGATTTTCTG | | LTVEQDIE | |
| | | CTGCATAATCTG | | EFTFLLIN | |
| | | TGGAAAGTTTGG | | GVCPGTQC | |
| | | CGTGAAACCATT | | | |
| | | TGTGGTGAAGCA | | | |
| | | TTTCGTTGTGTT | | | |
| | | ATTGCAGAAGCA | | | |
| | | CAGTTGGACCCT | | | |
| | | GTAACCCTGACC | | | |
| | | CAACTGAAAGAT | | | |
| | | CAGTTTATGGAA | | | |
| | | CGTCGTCGTGAG | | | |
| | | ATACCGAAAAAA | | | |
| | | CTGGTTGAAGAT | | | |
| | | GCCATTAGCAAT | | | |
| | | GGTGAACTGCCG | | | |
| | | AAAGATATCAAT | | | |
| | | CGTGAACTGCTG | | | |
| | | CTGGATATGATT | | | |
| | | TTTGGTTTTTGT | | | |
| | | TGGTATCGCCTG | | | |
| | | CTGACCGAACAG | | | |
| | | TTGACCGTTGAA | | | |
| | | CAGGATATTGAA | | | |
| | | GAATTTACCTTC | | | |
| | | CTGCTGATTAAT | | | |
| | | GGTGTTTGTCCG | | | |
| | | GGTACACAGTGT | | | |
| Ph1F | Ph10 | atgatacgaaac | 116 | N/A | |
| Operator | | gtaccgtatcgt | | | |
| | | taaggt | | | |
| Cro Binding | Cro | ATGGAACAACGC | 156 | MEQRITLK | 157 |
| Protein | | ATAACCCTGAAA | | DYAMRFGQ | |
| | | GATTATGCAATG | | TKTAKDLG | |
| | | CGCTTTGGGCAA | | VYQSAINK | |
| | | ACCAAGACAGCT | | AIHAGRKI | |
| | | AAAGATCTCGGC | | FLTINADG | |
| | | GTATATCAAAGC | | SVYAEEVK | |
| | | GCGATCAACAAG | | PFPSNKKT | |
| | | GCCATTCATGCA | | TA | |
| | | GGCCGAAAGATT | | | |
| | | TTTTTAACTATA | | | |
| | | AACGCTGATGGA | | | |
| | | AGCGTTTATGCG | | | |
| | | GAAGAGGTAAAG | | | |
| | | CCCTTCCCGAGT | | | |
| | | AACAAAAAAACA | | | |
| | | ACAGCA | | | |
| scCro | | ATGGAACAACGC | 158 | MEQRITLK | 159 |
| Binding | | ATAACCCTGAAA | | DYAMRFGQ | |
| Protein | | GATTATGCAATG | | TKTAKDLG | |
| | | CGCTTTGGGCAA | | VYQSAINK | |
| | | ACCAAGACAGCT | | AIHAGRKI | |
| | | AAAGATCTCGGC | | FLTINADG | |
| | | GTATATCAAAGC | | SVYAEEVK | |
| | | GCGATCAACAAG | | PFPSNKKT | |
| | | GCCATTCATGCA | | TAAGTGGS | |
| | | GGCCGAAAGATT | | GGMEQRIT | |
| | | TTTTTAACTATA | | LKDYAMRF | |
| | | AACGCTGATGGA | | GQTKTAKD | |
| | | AGCGTTTATGCG | | LGVYQSAI | |
| | | GAAGAGGTAAAG | | NKAIHAGR | |
| | | CCCTTCCCGAGT | | KIFLTINA | |
| | | AACAAAAAAACA | | DGSVYAEE | |
| | | ACAGCAGCCGGT | | VKPFPSNK | |
| | | ACCGGTGGCTCT | | KTTA | |
| | | GGCGGCATGGAA | | | |
| | | CAACGCATAACC | | | |
| | | CTGAAAGATTAT | | | |
| | | GCAATGCGCTTT | | | |
| | | GGGCAAACCAAG | | | |
| | | ACAGCTAAAGAT | | | |
| | | CTCGGCGTATAT | | | |
| | | CAAAGCGCGATC | | | |
| | | AACAAGGCCATT | | | |
| | | CATGCAGGCCGA | | | |
| | | AAGATTTTTTTA | | | |
| | | ACTATAAACGCT | | | |
| | | GATGGAAGCGTT | | | |
| | | TATGCGGAAGAG | | | |
| | | GTAAAGCCCTTC | | | |
| | | CCGAGTAACAAA | | | |
| | | AAAACAACAGCA | | | |
| Cro Operator | OR3 | TATCACCGCAAG | 117 | N/A | |
| 3 | | GGATA | | | |
| RpoA N- | RpoA | ATGCAGGGTTCT | 88 | MQGSVTEF | 89 |
| Terminal | | GTGACAGAGTTT | | LKPRLVDI | |
| Domain | | CTAAAACCGCGC | | EQVSSTHA | |
| | | CTGGTTGATATC | | KVTLEPLE | |
| | | GAGCAAGTGAGT | | RGFGHTLG | |
| | | TCGACGCACGCC | | NALRRILL | |
| | | AAGGTGACCCTT | | SSMPGCAV | |
| | | GAGCCTTTAGAG | | TEVEIDGV | |
| | | CGTGGCTTTGGC | | LHEYSTKE | |
| | | CATACTCTGGGT | | GVQEDILE | |
| | | AACGCACTGCGC | | ILLNLKGL | |
| | | CGTATTCTGCTC | | AVRVQGKD | |
| | | TCATCGATGCCG | | EVILTLNK | |
| | | GGTTGCGCGGTG | | SGIGPVTA | |
| | | ACCGAGGTTGAG | | ADITHDGD | |
| | | ATTGATGGTGTA | | VEIVKPQH | |
| | | CTACATGAGTAC | | VICHLTDE | |
| | | AGCACCAAAGAA | | NASISMRI | |
| | | GGCGTTCAGGAA | | KVQRGRGY | |
| | | GATATCCTGGAA | | VPASTRIH | |
| | | ATCCTGCTCAAC | | SEEDERPI | |
| | | CTGAAAGGGCTG | | GRLLVDAC | |
| | | GCGGTGAGAGTT | | YSPVERIA | |
| | | CAGGGCAAAGAT | | YNVEAARV | |
| | | GAAGTTATTCTT | | EQRTDLDK | |
| | | ACCTTGAATAAA | | LVIEMETN | |
| | | TCTGGCATTGGC | | GTIDPEEA | |
| | | CCTGTGACTGCA | | IRRAATIL | |
| | | GCCGATATCACC | | AEQLEAFV | |
| | | CACGACGGTGAT | | DLRDVRQP | |
| | | GTCGAAATCGTC | | EVKEEKPE | |
| | | AAGCCGCAGCAC | | | |
| | | GTGATCTGCCAC | | | |
| | | CTGACCGATGAG | | | |
| | | AACGCGTCTATT | | | |
| | | AGCATGCGTATC | | | |
| | | AAAGTTCAGCGC | | | |
| | | GGTCGTGGTTAT | | | |
| | | GTGCCGGCTTCT | | | |
| | | ACCCGAATTCAT | | | |
| | | TCGGAAGAAGAT | | | |
| | | GAGCGCCCAATC | | | |
| | | GGCCGTCTGCTG | | | |
| | | GTCGACGCATGC | | | |
| | | TACAGCCCTGTG | | | |
| | | GAGCGTATTGCC | | | |
| | | TACAATGTTGAA | | | |
| | | GCAGCGCGTGTA | | | |
| | | GAACAGCGTACC | | | |
| | | GACCTGGACAAG | | | |
| | | CTGGTCATCGAA | | | |
| | | ATGGAAACCAAC | | | |
| | | GGCACAATCGAT | | | |
| | | CCTGAAGAGGCG | | | |
| | | ATTCGTCGTGCG | | | |
| | | GCAACCATTCTG | | | |
| | | GCTGAACAACTG | | | |
| | | GAAGCTTTCGTT | | | |
| | | GACTTACGTGAT | | | |
| | | GTACGTCAGCCT | | | |
| | | GAAGTGAAAGAA | | | |
| | | GAGAAACCAGAG | | | |
| iLID LOV2- | iLID | GGGGAGTTTCTG | 43 | GEFLATTL | 44 |
| SsrA | | GCAACCACACTG | | ERIEKNFV | |
| | | GAACGGATCGAG | | ITDPRLPD | |
| | | AAAAATTTCGTG | | NPIIFASD | |
| | | ATTACTGATCCG | | SFLQLTEY | |
| | | AGACTGCCTGAC | | SREEILGR | |
| | | AACCCAATCATT | | NCRFLQGP | |
| | | TTTGCGAGCGAT | | ETDRATVR | |
| | | TCCTTCCTGCAG | | KIRDAIDN | |
| | | CTGACAGAATAT | | QTEVTVQL | |
| | | TCTCGGGAAGAG | | INYTKSGK | |
| | | ATCCTGGGGCGC | | KFWNVFHL | |
| | | AATTGCCGTTTT | | QPMRDYKG | |
| | | CTGCAGGGACCC | | DVQYFIGV | |
| | | GAGACAGACCGT | | QLDGTERL | |
| | | GCCACTGTTCGG | | HGAAEREA | |
| | | AAAATCAGAGAT | | VCLIKKTA | |
| | | GCTATTGACAAC | | FQIAEAAN | |
| | | CAGACTGAAGTG | | DENYF | |
| | | ACCGTTCAGCTG | | | |
| | | ATCAATTATACC | | | |
| | | AAGAGCGGCAAG | | | |
| | | AAGTTCTGGAAC | | | |
| | | GTGTTCCACCTG | | | |
| | | CAGCCGATGCGC | | | |
| | | GATTATAAGGGC | | | |
| | | GACGTCCAGTAC | | | |
| | | TTCATTGGCGTG | | | |
| | | CAGCTGGATGGC | | | |
| | | ACCGAACGTCTT | | | |
| | | CATGGCGCCGCT | | | |
| | | GAGCGTGAGGCG | | | |
| | | GTCTGCCTGATC | | | |
| | | AAAAAGACAGCC | | | |
| | | TTTCAGATTGCT | | | |
| | | GAGGCAGCGAAC | | | |
| | | GACGAAAATTAC | | | |
| | | TTTTAA | | | |
| SsrA Motif | SsrA | GAGGCAGCGAAC | 45 | EAANDENY | 46 |
| | | GACGAAAATTAC | | F | |
| | | TTT | | | |
| iLID SspB | SspB- | TCCAGCTCCCCG | 47 | SSSPKRPK | 48 |
| | nano | AAACGCCCTAAG | | LLREYYDW | |
| | | CTGCTGCGTGAA | | LVDNSFTP | |
| | | TATTACGATTGG | | YLVVDATY | |
| | | CTGGTTGATAAC | | LGVNVPVE | |
| | | AGCTTTACCCCA | | YVKDGQIV | |
| | | TATCTGGTGGTG | | LNLSASAT | |
| | | GATGCCACATAC | | GNLQLTND | |
| | | CTGGGCGTGAAC | | FIQFNARF | |
| | | GTGCCCGTGGAG | | KGVSRELY | |
| | | TATGTGAAAGAC | | IPMGAALA | |
| | | GGTCAGATCGTG | | IYARENGD | |
| | | CTGAATCTGTCT | | GVMFEPEE | |
| | | GCAAGTGCGACC | | IYDELNIG | |
| | | GGCAACCTGCAA | | | |
| | | CTGACAAATGAT | | | |
| | | TTTATCCAGTTC | | | |
| | | AACGCCCGCTTT | | | |
| | | AAGGGCGTGTCT | | | |
| | | CGTGAACTGTAT | | | |
| | | ATCCCGATGGGT | | | |
| | | GCCGCTCTGGCC | | | |
| | | ATTTACGCTCGC | | | |
| | | GAGAACGGCGAT | | | |
| | | GGTGTGATGTTC | | | |
| | | GAACCAGAAGAA | | | |
| | | ATCTATGACGAG | | | |
| | | CTGAATATTGGT | | | |
| | | TAA | | | |
| Ubiquitin | Ubi- | CAAATCTTTGTC | 53 | QIFVKTLT | 54 |
| | quitin | AAGACATTAACA | | GKTITLEV | |
| | | GGTAAGACCATC | | ESSDTIDN | |
| | | ACGTTGGAGGTA | | VKAKIQDK | |
| | | GAATCGAGTGAT | | EGIPPDQQ | |
| | | ACTATCGACAAT | | RLIFAGKQ | |
| | | GTAAAAGCAAAA | | LEDGRTLA | |
| | | ATCCAAGACAAG | | DYNIQKES | |
| | | GAGGGCATTCCC | | TLHLVLRL | |
| | | CCAGACCAGCAA | | RGG | |
| | | CGCTTGATTTTT | | | |
| | | GCGGGAAAGCAA | | | |
| | | CTTGAGGATGGC | | | |
| | | CGTACTTTAGCG | | | |
| | | GACTATAATATC | | | |
| | | CAGAAAGAATCT | | | |
| | | ACATTGCACTTA | | | |
| | | GTGTTGCGCCTG | | | |
| | | CGTGGGGGC | | | |
| GOI | LuxAB | ATGAAATTTGGA | 112 | MKFGNFLL | 34 |
| | | AACTTTTTGCTT | | TYQPPQFS | |
| | | ACATACCAACCT | | QTEVMKRL | |
| | | CCCCAATTTTCC | | VKLGRISE | |
| | | CAAACAGAGGTA | | ECGFDTVW | |
| | | ATGAAACGTTTG | | LLEHHFTE | |
| | | GTTAAATTAGGT | | FGLLGNPY | |
| | | CGCATCTCTGAG | | VAAAYLLG | |
| | | GAGTGTGGTTTT | | ATKKLNVG | |
| | | GATACCGTATGG | | TAAIVLPT | |
| | | TTACTGGAGCAT | | AHPVRQLE | |
| | | CATTTCACGGAG | | DVNLLDQM | |
| | | TTTGGTTTGCTT | | SKGRFRFG | |
| | | GGTAACCCTTAT | | ICRGLYNK | |
| | | GTCGCTGCTGCA | | DFRVFGTD | |
| | | TATTTACTTGGC | | MNNSRALA | |
| | | GCGACTAAAAAA | | ECWYGLIK | |
| | | TTGAATGTAGGA | | NGMTEGYM | |
| | | ACTGCCGCTATT | | EADNEHIK | |
| | | GTTCTTCCCACA | | FHKVKVNP | |
| | | GCCCATCCAGTA | | AAYSRGGA | |
| | | CGCCAACTTGAA | | PVYVVAES | |
| | | GATGTGAATTTA | | ASTTEWAA | |
| | | TTGGATCAAATG | | QFGLPMIL | |
| | | TCAAAAGGACGA | | SWIINTNE | |
| | | TTTCGGTTTGGT | | KKAQLELY | |
| | | ATTTGCCGAGGG | | NEVAQEYG | |
| | | CTTTACAACAAG | | HDIHNIDH | |
| | | GACTTTCGCGTA | | CLSYITSV | |
| | | TTCGGCACAGAT | | DHDSIKAK | |
| | | ATGAATAACAGT | | EICRKFLG | |
| | | CGCGCCTTAGCG | | HWYDSYVN | |
| | | GAATGCTGGTAC | | ATTIFDDS | |
| | | GGGCTGATAAAG | | DQTRGYDF | |
| | | AATGGCATGACA | | NKGQWRDF | |
| | | GAGGGATATATG | | VLKGHKDT | |
| | | GAAGCTGATAAT | | NRRIDYSY | |
| | | GAACATATCAAG | | EINPVGTP | |
| | | TTCCATAAGGTA | | QECIDIIQ | |
| | | AAAGTAAACCCC | | KDIDATGI | |
| | | GCGGCGTATAGC | | SNICCGFE | |
| | | AGAGGTGGCGCA | | ANGTVDEI | |
| | | CCGGTTTATGTG | | IASMKLFQ | |
| | | GTGGCTGAATCA | | SDVMPFLK | |
| | | GCTTCGACGACT | | EKQRSLLY | |
| | | GAGTGGGCTGCT | | YGGGGSGG | |
| | | CAATTTGGCCTA | | GGSGGGGS | |
| | | CCGATGATATTA | | GGGGSKFG | |
| | | AGTTGGATTATA | | LFFLNFIN | |
| | | AATACTAACGAA | | STTVQEQS | |
| | | AAGAAAGCACAA | | IVRMQEIT | |
| | | CTTGAGCTTTAT | | EYVDKLNF | |
| | | AATGAAGTGGCT | | EQILVYEN | |
| | | CAAGAATATGGG | | HFSDNGVV | |
| | | CACGATATTCAT | | GAPLTVSG | |
| | | AATATCGACCAT | | FLLGLTEK | |
| | | TGCTTATCATAT | | IKIGSLNH | |
| | | ATAACATCTGTA | | IITTHHPV | |
| | | GATCATGACTCA | | RIAEEACL | |
| | | ATTAAAGCGAAA | | LDQLSEGR | |
| | | GAGATTTGCCGG | | FILGFSDC | |
| | | AAATTTCTGGGG | | EKKDEMHF | |
| | | CATTGGTATGAT | | FNRPVEYQ | |
| | | TCTTATGTGAAT | | QQLFEECY | |
| | | GCTACGACTATT | | EIINDALT | |
| | | TTTGATGATTCA | | TGYCNPDN | |
| | | GACCAAACAAGA | | DFYSFPKI | |
| | | GGTTATGATTTC | | SVNPHAYT | |
| | | AATAAAGGGCAG | | PGGPRKYV | |
| | | TGGCGTGACTTT | | TATSHHIV | |
| | | GTATTAAAAGGA | | EWAAKKGI | |
| | | CATAAAGATACT | | PLIFKWDD | |
| | | AATCGCCGTATT | | SNDVRYEY | |
| | | GATTACAGTTAC | | AERYKAVA | |
| | | GAAATCAATCCC | | DKYDVDLS | |
| | | GTGGGAACGCCG | | EIDHQLMI | |
| | | CAGGAATGTATT | | LVNYNEDS | |
| | | GACATAATTCAA | | NKAKQETR | |
| | | AAAGACATTGAT | | AFISDYVL | |
| | | GCTACAGGAATA | | EMHPNENF | |
| | | TCAAATATTTGT | | ENKLEEII | |
| | | TGTGGATTTGAA | | AENAVGNY | |
| | | GCTAATGGAACA | | TECITAAK | |
| | | GTAGACGAAATT | | LAIEKCGA | |
| | | ATTGCTTCCATG | | KSVLLSFE | |
| | | AAGCTCTTCCAG | | PMNDLMSQ | |
| | | TCTGATGTCATG | | KNVINIVD | |
| | | CCATTTCTTAAA | | DNIKKYHT | |
| | | GAAAAACAACGT | | EYT* | |
| | | TCGCTATTATAT | | | |
| | | TATGGCGGTGGC | | | |
| | | GGTAGCGGCGGT | | | |
| | | GGCGGTAGCGGC | | | |
| | | GGTGGCGGTAGC | | | |
| | | GGCGGTGGCGGT | | | |
| | | AGCAAATTTGGA | | | |
| | | TTGTTCTTCCTT | | | |
| | | AACTTCATCAAT | | | |
| | | TCAACAACTGTT | | | |
| | | CAAGAACAGAGT | | | |
| | | ATAGTTCGCATG | | | |
| | | CAGGAAATAACG | | | |
| | | GAGTATGTTGAT | | | |
| | | AAGTTGAATTTT | | | |
| | | GAACAGATTTTA | | | |
| | | GTGTATGAAAAT | | | |
| | | CATTTTTCAGAT | | | |
| | | AATGGTGTTGTC | | | |
| | | GGCGCTCCTCTG | | | |
| | | ACTGTTTCTGGT | | | |
| | | TTTCTGCTCGGT | | | |
| | | TTAACAGAGAAA | | | |
| | | ATTAAAATTGGT | | | |
| | | TCATTAAATCAC | | | |
| | | ATCATTACAACT | | | |
| | | CATCATCCTGTC | | | |
| | | CGCATAGCGGAG | | | |
| | | GAAGCTTGCTTA | | | |
| | | TTGGATCAGTTA | | | |
| | | AGTGAAGGGAGA | | | |
| | | TTTATTTTAGGG | | | |
| | | TTTAGTGATTGC | | | |
| | | GAAAAAAAAGAT | | | |
| | | GAAATGCATTTT | | | |
| | | TTTAATCGCCCG | | | |
| | | GTTGAATATCAA | | | |
| | | CAGCAACTATTT | | | |
| | | GAAGAGTGTTAT | | | |
| | | GAAATCATTAAC | | | |
| | | GATGCTTTAACA | | | |
| | | ACAGGCTATTGT | | | |
| | | AATCCAGATAAC | | | |
| | | GATTTTTATAGC | | | |
| | | TTCCCTAAAATA | | | |
| | | TCTGTAAATCCC | | | |
| | | CATGCTTATACG | | | |
| | | CCAGGCGGACCT | | | |
| | | CGGAAATATGTA | | | |
| | | ACAGCAACCAGT | | | |
| | | CATCATATTGTT | | | |
| | | GAGTGGGCGGCC | | | |
| | | AAAAAAGGTATT | | | |
| | | CCTCTCATCTTT | | | |
| | | AAGTGGGATGAT | | | |
| | | TCTAATGATGTT | | | |
| | | AGATATGAATAT | | | |
| | | GCTGAAAGATAT | | | |
| | | AAAGCCGTTGCG | | | |
| | | GATAAATATGAC | | | |
| | | GTTGACCTATCA | | | |
| | | GAGATAGACCAT | | | |
| | | CAGTTAATGATA | | | |
| | | TTAGTTAACTAT | | | |
| | | AACGAAGATAGT | | | |
| | | AATAAAGCTAAA | | | |
| | | CAAGAGACGCGT | | | |
| | | GCATTTATTAGT | | | |
| | | GATTATGTTCTT | | | |
| | | GAAATGCACCCT | | | |
| | | AATGAAAATTTC | | | |
| | | GAAAATAAACTT | | | |
| | | GAAGAAATAATT | | | |
| | | GCAGAAAACGCT | | | |
| | | GTCGGAAATTAT | | | |
| | | ACGGAGTGTATA | | | |
| | | ACTGCGGCTAAG | | | |
| | | TTGGCAATTGAA | | | |
| | | AAGTGTGGTGCG | | | |
| | | AAAAGTGTATTG | | | |
| | | CTGTCCTTTGAA | | | |
| | | CCAATGAATGAT | | | |
| | | TTGATGAGCCAA | | | |
| | | AAAAATGTAATC | | | |
| | | AATATTGTTGAT | | | |
| | | GATAATATTAAG | | | |
| | | AAGTACCACACG | | | |
| | | GAATATACCTAA | | | |
| GOI | SpecR | ATGAGGGAAGCG | 79 | MREAVIAE | 80 |
| | | GTGATCGCCGAA | | VSTQLSEV | |
| | | GTATCGACTCAA | | VGVIERHL | |
| | | CTATCAGAGGTA | | EPTLLAVH | |
| | | GTTGGCGTCATC | | LYGSAVDG | |
| | | GAGCGCCATCTC | | GLKPHSDI | |
| | | GAACCGACGTTG | | DLLVTVTV | |
| | | CTGGCCGTACAT | | RLDETTRR | |
| | | TTGTACGGCTCC | | ALINDLLE | |
| | | GCAGTGGATGGC | | TSASPGES | |
| | | GGCCTGAAGCCA | | EILRAVEV | |
| | | CACAGTGATATT | | TIVVHDDI | |
| | | GATTTGCTGGTT | | IPWRYPAK | |
| | | ACGGTGACCGTA | | RELQFGEW | |
| | | AGGCTTGATGAA | | QRNDILAG | |
| | | ACAACGCGGCGA | | IFEPATID | |
| | | GCTTTGATCAAC | | IDLAILLT | |
| | | GACCTTTTGGAA | | KAREHSVA | |
| | | ACTTCGGCTTCC | | LVGPAAEE | |
| | | CCTGGAGAGAGC | | LFDPVPEQ | |
| | | GAGATTCTCCGC | | DLFEALNE | |
| | | GCTGTAGAAGTC | | TLTLWNSP | |
| | | ACCATTGTTGTG | | PDWAGDER | |
| | | CACGACGACATC | | NVVLTLSR | |
| | | ATTCCGTGGCGT | | IWYSAVTG | |
| | | TATCCAGCTAAG | | KIAPKDVA | |
| | | CGCGAACTGCAA | | ADWAMERL | |
| | | TTTGGAGAATGG | | PAQYQPVI | |
| | | CAGCGCAATGAC | | LEARQAYL | |
| | | ATTCTTGCAGGT | | GQEEDRLA | |
| | | ATCTTCGAGCCA | | SRADQLEE | |
| | | GCCACGATCGAC | | FVHYVKGE | |
| | | ATTGATCTGGCT | | ITKVVGK* | |
| | | ATCTTGCTGACA | | | |
| | | AAAGCAAGAGAA | | | |
| | | CATAGCGTTGCC | | | |
| | | TTGGTAGGTCCA | | | |
| | | GCGGCGGAGGAA | | | |
| | | CTCTTTGATCCG | | | |
| | | GTTCCTGAACAG | | | |
| | | GATCTATTTGAG | | | |
| | | GCGCTAAATGAA | | | |
| | | ACCTTAACGCTA | | | |
| | | TGGAACTCGCCG | | | |
| | | CCCGACTGGGCT | | | |
| | | GGCGATGAGCGA | | | |
| | | AATGTAGTGCTT | | | |
| | | ACGTTGTCCCGC | | | |
| | | ATTTGGTACAGC | | | |
| | | GCAGTAACCGGC | | | |
| | | AAAATCGCGCCG | | | |
| | | AAGGATGTCGCT | | | |
| | | GCCGACTGGGCA | | | |
| | | ATGGAGCGCCTG | | | |
| | | CCGGCCCAGTAT | | | |
| | | CAGCCCGTCATA | | | |
| | | CTTGAAGCTAGA | | | |
| | | CAGGCTTATCTT | | | |
| | | GGACAAGAAGAA | | | |
| | | GATCGCTTGGCC | | | |
| | | TCGCGCGCAGAT | | | |
| | | CAGTTGGAAGAA | | | |
| | | TTTGTCCACTAC | | | |
| | | GTGAAAGGCGAG | | | |
| | | ATCACCAAGGTA | | | |
| | | GTCGGCAAATGA | | | |
| GOI | GFPuv | N/A | | MSKGEELF | 160 |
| | | | | TGVVPILV | |
| | | | | ELDGDVNG | |
| | | | | HKFSVSGE | |
| | | | | GEGDATYG | |
| | | | | KLTLKFIC | |
| | | | | TTGKLPVP | |
| | | | | WPTLVTTF | |
| | | | | SYGVQCFS | |
| | | | | RYPDHMKR | |
| | | | | HDFFKSAM | |
| | | | | PEGYVQER | |
| | | | | TISFKDDG | |
| | | | | NYKTRAEV | |
| | | | | KFEGDTLV | |
| | | | | NRIELKGI | |
| | | | | DFKEDGNI | |
| | | | | LGHKLEYN | |
| | | | | YNSHNVYI | |
| | | | | TADKQKNG | |
| | | | | IKANFKIR | |
| | | | | HNIEDGSV | |
| | | | | QLADHYQQ | |
| | | | | NTPIGDGP | |
| | | | | VLLPDNHY | |
| | | | | LSTQSALS | |
| | | | | KDPNEKRD | |
| | | | | HMVLLEFV | |
| | | | | TAAGITHG | |
| | | | | MDELYK | |
| GOI | T7 RNA | | | MNTINIAK | 161 |
| | Polymer- | | | NDFSDIEL | |
| | ase | | | AAIPFNTL | |
| | | | | ADHYGERL | |
| | | | | AREQLALE | |
| | | | | HESYEMGE | |
| | | | | ARFRKMFE | |
| | | | | RQLKAGEV | |
| | | | | ADNAAAKP | |
| | | | | LITTLLPK | |
| | | | | MIARINDW | |
| | | | | FEEVKAKR | |
| | | | | GKRPTAFQ | |
| | | | | FLQEIKPE | |
| | | | | AVAYITIK | |
| | | | | TTLACLTS | |
| | | | | ADNTTVQA | |
| | | | | VASAIGRA | |
| | | | | IEDEARFG | |
| | | | | RIRDLEAK | |
| | | | | HFKKNVEE | |
| | | | | QLNKRVGH | |
| | | | | VYKKAFMQ | |
| | | | | VVEADMLS | |
| | | | | KGLLGGEA | |
| | | | | WSSWHKED | |
| | | | | SIHVGVRC | |
| | | | | IEMLIEST | |
| | | | | GMVSLHRQ | |
| | | | | NAGVVGQD | |
| | | | | SETIELAP | |
| | | | | EYAEAIAT | |
| | | | | RAGALAGI | |
| | | | | SPMFQPCV | |
| | | | | VPPKPWTG | |
| | | | | ITGGGYWA | |
| | | | | NGRRPLAL | |
| | | | | VRTHSKKA | |
| | | | | LMRYEDVY | |
| | | | | MPEVYKAI | |
| | | | | NIAQNTAW | |
| | | | | KINKKVLA | |
| | | | | VANVITKW | |
| | | | | KHCPVEDI | |
| | | | | PAIEREEL | |
| | | | | PMKPEDID | |
| | | | | MNPEALTA | |
| | | | | WKRAAAAV | |
| | | | | YRKDKARK | |
| | | | | SRRISLEF | |
| | | | | MLEQANKF | |
| | | | | ANHKAIWF | |
| | | | | PYNMDWRG | |
| | | | | RVYAVSMF | |
| | | | | NPQGNDMT | |
| | | | | KGLLTLAK | |
| | | | | GKPIGKEG | |
| | | | | YYWLKIHG | |
| | | | | ANCAGVDK | |
| | | | | VPFPERIK | |
| | | | | FIEENHEN | |
| | | | | IMACAKSP | |
| | | | | LENTWWAE | |
| | | | | QDSPFCFL | |
| | | | | AFCFEYAG | |
| | | | | VQHHGLSY | |
| | | | | NCSLPLAF | |
| | | | | DGSCSGIQ | |
| | | | | HFSAMLRD | |
| | | | | EVGGRAVN | |
| | | | | LLPSETVQ | |
| | | | | DIYGIVAK | |
| | | | | KVNEILQA | |
| | | | | DAINGTDN | |
| | | | | EVVTVTDE | |
| | | | | NTGEISEK | |
| | | | | VKLGTKAL | |
| | | | | AGQWLAYG | |
| | | | | VTRSVTKR | |
| | | | | SVMTLAYG | |
| | | | | SKEFGFRQ | |
| | | | | QVLEDTIQ | |
| | | | | PAIDSGKG | |
| | | | | LMFTQPNQ | |
| | | | | AAGYMAKL | |
| | | | | IWESVSVT | |
| | | | | VVAAVEAM | |
| | | | | NWLKSAAK | |
| | | | | LLAAEVKD | |
| | | | | KKTGEILR | |
| | | | | KRCAVHWV | |
| | | | | TPDGFPVW | |
| | | | | QEYKKPIQ | |
| | | | | TRLNLMFL | |
| | | | | GQFRLQPT | |
| | | | | INTNKDSE | |
| | | | | IDAHKQES | |
| | | | | GIAPNFVH | |
| | | | | SQDGSHLR | |
| | | | | KTVVWAHE | |
| | | | | KYGIESFA | |
| | | | | LIHDSFGT | |
| | | | | IPADAANL | |
| | | | | FKAVRETM | |
| | | | | VDTYESCD | |
| | | | | VLADFYDQ | |
| | | | | FADQLHES | |
| | | | | QLDKMPAL | |
| | | | | PAKGNLNL | |
| | | | | RDILESDF | |
| | | | | AFA | |

TABLE 22
Primers.

| | | SEQ | | SEQ |
|---|---|---|---|---|
| | | ID | | ID |
| Component | F Primer | NO. | R Primer | NO. |
| HIVpro_{0A} | TACTGGCCGAAGCCA | 170 | TGCCATGGCTTCG | 171 |
| substrate | TGGCAGCTGCGGAAC | | GCCAGTACGCGAG | |
| | CGCAGTATGAAGA | | CTTTACGACGACC | |
| | | | TTCAGCAATA | |
| 3CLpro_{0A} | TACAATCAGGGTTCC | 172 | AGCTGCACGGAAC | 173 |
| substrate | GTGCAGCTGCGGAAC | | CCTGATTGTAAAA | |
| | CGCAGTATGAAGAAA | | CTGCACGACGACC | |
| | TTC | | TTCAGCAATA | |
| HIVpro_{2A} | TGGCAGCTGCAGCTG | 174 | TGGCTTCGGCCAG | 175 |
| substrate | CGGAACCGCAGTATG | | TACGCGAGCTTTA | |
| (step 1) | AAGAAATTCCGATTT | | GCTGCACGACGAC | |
| | ATCT | | CTTCAGCAATA | |
| HIVpro_{2A} | TCGCGTACTGGCCGA | 176 | GTTCCGCAGCTGC | 177 |
| substrate | AGCCATGGCAGCTGC | | AGCTGCCATGGCT | |
| (step 2) | AGCTGCGGA | | TCGGCCAGTACGC | |
| | | | GAG | |
| 3CLpro_{2A} | CAGCTGCAGCTGCGG | 178 | CACGGAACCCTGA | 179 |
| substrate | AACCGCAGTATGAAG | | TTGTAAAACTGCA | |
| (step 1) | AAATTCCGATTTATC | | GCTGCACGACGAC | |
| | T | | CTTCAGCAATA | |
| 3CLpro_{2A} | TGCAGTTTTACAATC | 180 | ACTGCGGTTCCGC | 181 |
| substrate | AGGGTTCCGTGCAGC | | AGCTGCAGCTGCA | |
| (step 2) | TGCAGCTGCGGAACC | | CGGAACCCTGATT | |
| | GCAGT | | GTAAAACTGCA | |
| HIVpro_{4A} | ATGGCAGCTGCAGCT | 182 | CGGCCAGTACGCG | 183 |
| substrate | GCAGCTGCGGAACCG | | AGCTTTAGCTGCA | |
| (step 1) | CAGTATGAAGAAAT | | GCTGCACGACGAC | |
| | | | CTTCAGCAATA | |
| HIVpro_{4A} | TGCAGCTAAAGCTCG | 184 | TGCAGCTGCAGCT | 185 |
| substrate | CGTACTGGCCGAAGC | | GCCATGGCTTCGG | |
| (step 2) | CATGGCAGCTGCAGC | | CCAGTACGCGAGC | |
| | TGCA | | TTTAGCTGCA | |
| 3CLpro_{4A} | GTTCCGTGCAGCTGC | 186 | ACCCTGATTGTAA | 187 |
| substrate | AGCTGCAGCTGCGGA | | AACTGCAGCTGCA | |
| (step 1) | ACCGCAGTATGAAGA | | GCTGCACGACGAC | |
| | AAT | | CTTCAGCAATA | |
| 3CLpro_{4A} | AGCTGCAGTTTTACA | 188 | AGCTGCAGCTGCA | 189 |
| substrate | ATCAGGGTTCCGTGC | | CGGAACCCTGATT | |
| (step 2) | AGCTGCAGCT | | GTAAAACTGCAGC | |
| | | | T | |
| HIVpro | AGCTGAAAGAAGCGC | 140 | ACGGTATCATCCG | 141 |
| D25N | TGCTGAACACCGGCG | | CGCCGGTGTTCAG | |
| | CGGATGATACCGT | | CAGCGCTTCTTTC | |
| | | | AGCT | |
| 3CLpro | TGATGTAGTTTATTG | 143 | TATCCTCACTTGT | 142 |
| H41A | TCCTCGCGCAGTTAT | | GCAAATAACTGCG | |
| | TTGCACAAGTGAGGA | | CGAGGACAATAAA | |
| | TA | | CTACATCA | |
| USP7 | GCTTAAAAAACCAGG | 190 | GTAGCGCCCTGGT | 191 |
| C223S | GCGCTACAAGCTATA | | TTTTTAAGC | |
| | TGAATTCGCTGTTAC | | | |
| | AG | | | |
| PLpro | CTGACAAGCATTAAA | 192 | CAGCCCATTTAAT | 193 |
| C111S | TGGGCTGATAATAAT | | GCTTGTCAG | |
| | AGCTATCTGGCTACA | | | |
| | GCATTATTAACATTG | | | |
| pBAD₂_HIV | ATATGGTCTCACATG | 194 | ATATGGTCTCATT | 195 |
| | GCGGATCGCCAGG | | TAAAAGTTCAGGG | |
| | | | TGCAGCC | |
| pBAD₂_3CL | TCGAGCTCTTAAAGA | 196 | ATCCGCCAAAACA | 197 |
| | GGAGAAAGGTCATGT | | GCCAAGCTTTTAT | |
| | CGGGGTTCCGTAAAA | | TGGAAAGTTACGC | |
| | T | | CGCTACA | |
| pBAD₂_USP7 | AGCTCTTAAAGAGGA | 198 | CAATGATGATGAT | 199 |
| | GAAAGGTCATGAGTA | | GATGATGGTTGTG | |
| | AAAAGCATACAGGGT | | AATTTTGATAGCC | |
| | | | TTTTC | |
| pBAD₂_PL | AGCTCTTAAAGAGGA | 200 | TCCGCCAAAACAG | 201 |
| | GAAAGGTCATGGAGG | | CCAAGCTTTTACT | |
| | TTCGTACTATTAAGG | | TGATCGTCGTGG | |
| | T | | TGTAAGAGT | |
| PLpro_{0A} | GGAACCGCAGTATGA | 202 | TCTTCATACTGCG | 203 |
| substrate | AGAAATTCCGATTTA | | GTTCCGCAGCTGC | |
| | TCTGT | | CCCCCCACGTAAA | |
| | | | CGACGACCTTCAG | |
| | | | CAATAG | |
| PLpro_{4A} | CATTACGTGGTGGAG | 204 | TGCTCCACCACGT | 205 |
| substrate | CAGCAGCAGCAGCAG | | AATGCTGCTGCTG | |
| | CTGCGGAACCGCAGT | | CACGACGACCTT | |
| | A | | CAGCAAT | |
| RBS_{3CL} | CGATGAGAAGGATGT | 206 | TTTTTTTAGGGCC | 207 |
| | CAGTGTGTAATCGAC | | CTACTGACTGTTA | |
| | AGCAGCGGATAAGGA | | TTGGAAAGTTACG | |
| | GGTATTAATGTCGGG | | CCGCTACATTG | |
| | GTTCCGTAAAATGG | | | |
| RBS_{HIV} | ACAGTAACATAGGGG | 208 | ATTAATCCTCCCC | 209 |
| | AGGATTAATATGGCG | | TATGTTACTGTGG | |
| | GATCGCCAGGGCACC | | ATATTACACACT | |
| | GTGA | | GACATCCTTCT | |
| RBS_{USP} | CATATCCTAAGCGCT | 210 | GTTTTTTTTTAGG | 211 |
| | TCTTAAATGAGTAAA | | GCCCTACTGACTG | |
| | AAGCATACAGGGTAC | | TTAGTTGTGAATT | |
| | GTGG | | TTGATAGCCTTTT | |
| | | | CCAG | |
| RBS_{PL}_library | CGATGAGAAGGATGT | 212 | TAGGGCCCTACTG | 213 |
| | CAGTGTGTAAATKTG | | ACTGTTACTTGAT | |
| | CSAMCMTTAAAGGAG | | CGTCGTGGTGTAA | |
| | STTCCAAATGGAGGT | | G | |
| | TCGTACTATTAAGG | | | |

TABLE 24
RBS Library

| RBS | | SEQ | Vari- | min | max |
|---|---|---|---|---|---|
| library | Sequence | ID | ants | TIR | TIR |
| “L2-GOI | RGRCCGACDGW | | 48 | 67.63 | 172057.1 |
| RBS” | MGGTATAATAA | | | | |
| pET/T7 | KSAACTYGSAG | | 96 | 3071 | 70645.25 |
| RBS | BAGGAK | | | | |
| “L1- | | | | | |
| T7RBS” | | | | | |

TABLE 25
RBS sequences and predicted TIR

| | | SEQ | | Screen RFU/ |
|---|---|---|---|---|
| RBS | Sequence | ID | TIR (au) | OD600 |
| Wt | GTGCAGTAAGGA | | 142911.82 | N/A |
| GOI | GGAAAAAAAA | | | |
| L2- | GGACCGACAAGC | | 1022.05 | 834027740 |
| 60 | GGTATAATAA | | | |
| L2- | GGGCCGACAAGC | | 217.33 | 1168187617 |
| 71 | GGTATAATAA | | | |
| L2- | AGACCGACTTGC | | 239.54 | 1059852943 |
| 74 | GGTATAATAA | | | |
| L2- | GGGCCGACTTGC | | 205.90 | 960016718.3 |
| 93 | GGTATAATAA | | | |
| L2- | GGGCCGACTGGA | | 11717.20 | 765599354 |
| 94 | GGTATAATAA | | | |
| L2- | GGGCCGACTGGA | | 11717.20 | 1078302799 |
| 96 | GGTATAATAA | | | |

TABLE 26
Vector/RBS Starting TIR

| Vector/RBS | Starting TIR (au) |
|---|---|
| pET16b-GFPuv/T7RBS | 25904 |
| T7optC215S/GOI RBS | 142911 |

TABLE 27
Modified enzyme sequences

| | Modifications | | |
|---|---|---|---|
| | (Residues | | |
| Enzyme | removed) | WT sequence | SEQ ID |
| PTP1B | Truncate 322- | MEMEKEFEQIDKSGSWAAIYQDIRHEASDFPC | 235 |
| | 435 | RVAKLPKNKNRNRYRDVSPFDHSRIKLHQED | |
| | | NDYINASLIKMEEAQRSYILTQGPLPNTCGHF | |
| | | WEMVWEQKSRGVVMLNRVMEKGSLKCAQY | |
| | | WPQKEEKEMIFEDTNLKLTLISEDIKSYYTVRQ | |
| | | LELENLTTQETREILHFHYTTWPDFGVPESPAS | |
| | | FLNFLFKVRESGSLSPEHGPVVVHCSAGIGRSG | |
| | | TFCLADTCLLLMDKRKDPSSVDIKKVLLEMRK | |
| | | FRMGLIQTADQLRFSYLAVIEGAKFIMGDSSV | |
| | | QDQWKELSHEDLEPPPEHIPPPPRPPKRILEPHN | |
| | | GKCREFFPNHQWVKEETQEDKDCPIKEEKGSP | |
| | | LNAAPYGIESMSQDTEVRSRVVGGSLRGAQA | |
| | | ASPAKGEPSLPEKDEDHALSYWKPFLVNMCV | |
| | | ATVLTAGAYLCYRFLFNSNT | |
| PTP1B | Truncate 406- | MEMEKEFEQIDKSGSWAAIYQDIRHEASDFPC | 236 |
| | 435 | RVAKLPKNKNRNRYRDVSPFDHSRIKLHQED | |
| | | NDYINASLIKMEEAQRSYILTQGPLPNTCGHF | |
| | | WEMVWEQKSRGVVMLNRVMEKGSLKCAQY | |
| | | WPQKEEKEMIFEDTNLKLTLISEDIKSYYTVRQ | |
| | | LELENLTTQETREILHFHYTTWPDFGVPESPAS | |
| | | FLNFLFKVRESGSLSPEHGPVVVHCSAGIGRSG | |
| | | TFCLADTCLLLMDKRKDPSSVDIKKVLLEMRK | |
| | | FRMGLIQTADQLRFSYLAVIEGAKFIMGDSSV | |
| | | QDQWKELSHEDLEPPPEHIPPPPRPPKRILEPHN | |
| | | GKCREFFPNHQWVKEETQEDKDCPIKEEKGSP | |
| | | LNAAPYGIESMSQDTEVRSRVVGGSLRGAQA | |
| | | ASPAKGEPSLPEKDEDHALSYWKPFLVNMCV | |
| | | ATVLTAGAYLCYRFLFNSNT | |
| TCPTP | Truncate 318- | MPTTIEREFEELDTQRRWQPLYLEIRNESHDYP | 237 |
| | 415 | HRVAKFPENRNRNRYRDVSPYDHSRVKLQNA | |
| | | ENDYINASLVDIEEAQRSYILTQGPLPNTCCHF | |
| | | WLMVWQQKTKAVVMLNRIVEKESVKCAQY | |
| | | WPTDDQEMLFKETGFSVKLLSEDVKSYYTVH | |
| | | LLQLENINSGETRTISHFHYTTWPDFGVPESPA | |
| | | SFLNFLFKVRESGSLNPDHGPAVIHCSAGIGRS | |
| | | GTFSLVDTCLVLMEKGDDINIKQVLLNMRKY | |
| | | RMGLIQTPDQLRFSYMAIIEGAKCIKGDSSIQK | |
| | | RWKELSKEDLSPAFDHSPNKIMTEKYNGNRIG | |
| | | LEEEKLTGDRCTGLSSKMQDTMEENSESALRK | |
| | | RIREDRKATTAQKVQQMKQRLNENERKRKR | |
| | | WLYWQPILTKMGFMSVILVGAFVGWTLFFQQ | |
| | | NAL | |
| TCPTP | Truncate 388- | MPTTIEREFEELDTQRRWQPLYLEIRNESHDYP | 238 |
| | 415 | HRVAKFPENRNRNRYRDVSPYDHSRVKLQNA | |
| | | ENDYINASLVDIEEAQRSYILTQGPLPNTCCHF | |
| | | WLMVWQQKTKAVVMLNRIVEKESVKCAQY | |
| | | WPTDDQEMLFKETGFSVKLLSEDVKSYYTVH | |
| | | LLQLENINSGETRTISHFHYTTWPDFGVPESPA | |
| | | SFLNFLFKVRESGSLNPDHGPAVIHCSAGIGRS | |
| | | GTFSLVDTCLVLMEKGDDINIKQVLLNMRKY | |
| | | RMGLIQTPDQLRFSYMAIIEGAKCIKGDSSIQK | |
| | | RWKELSKEDLSPAFDHSPNKIMTEKYNGNRIG | |
| | | LEEEKLTGDRCTGLSSKMQDTMEENSESALRK | |
| | | RIREDRKATTAQKVQQMKQRLNENERKRKR | |
| | | WLYWQPILTKMGFMSVILVGAFVGWTLFFQQ | |
| | | NAL | |
| PTPRB | Truncate 1- | MLSHGAGLALWITLSLLQTGLAEPERCNFTLA | 239 |
| | 1662 | ESKASSHSVSIQWRILGSPCNFSLIYSSDTLGAA | |
| | | LCPTFRIDNTTYGCNLQDLQAGTIYNFRIISLDE | |
| | | ERTVVLQTDPLPPARFGVSKEKTTSTSLHVWW | |
| | | TPSSGKVTSYEVQLFDENNQKIQGVQIQESTS | |
| | | WNEYTFFNLTAGSKYNIAITAVSGGKRSFSVY | |
| | | TNGSTVPSPVKDIGISTKANSLLISWSHGSGNV | |
| | | ERYRLMLMDKGILVHGGVVDKHATSYAFHGL | |
| | | TPGYLYNLTVMTEAAGLQNYRWKLVRTAPM | |
| | | EVSNLKVTNDGSLTSLKVKWQRPPGNVDSYNI | |
| | | TLSHKGTIKESRVLAPWITETHFKELVPGRLYQ | |
| | | VTVSCVSGELSAQKMAVGRTFPDKVANLEAN | |
| | | NNGRMRSLVVSWSPPAGDWEQYRILLFNDSV | |
| | | VLLNITVGKEETQYVMDDTGLVPGRQYEVEVI | |
| | | VESGNLKNSERCQGRTVPLAVLQLRVKHANE | |
| | | TSLSIMWQTPVAEWEKYIISLADRDLLLIHKSL | |
| | | SKDAKEFTFTDLVPGRKYMATVTSISGDLKNS | |
| | | SSVKGRTVPAQVTDLHVANQGMTSSLFTNWT | |
| | | QAQGDVEFYQVLLIHENVVIKNESISSETSRYS | |
| | | FHSLKSGSLYSVVVTTVSGGISSRQVVVEGRT | |
| | | VPSSVSGVTVNNSGRNDYLSVSWLLAPGDVD | |
| | | NYEVTLSHDGKVVQSLVIAKSVRECSFSSLTPG | |
| | | RLYTVTITTRSGKYENHSFSQERTVPDKVQGV | |
| | | SVSNSARSDYLRVSWVHATGDFDHYEVTIKN | |
| | | KNNFIQTKSIPKSENECVFVQLVPGRLYSVTVT | |
| | | TKSGQYEANEQGNGRTIPEPVKDLTLRNRSTE | |
| | | DLHVTWSGANGDVDQYEIQLLFNDMKVFPPF | |
| | | HLVNTATEYRFTSLTPGRQYKILVLTISGDVQQ | |
| | | SAFIEGFTVPSAVKNIHISPNGATDSLTVNWTP | |
| | | GGGDVDSYTVSAFRHSQKVDSQTIPKHVFEHT | |
| | | FHRLEAGEQYQIMIASVSGSLKNQINVVGRTV | |
| | | PASVQGVIADNAYSSYSLIVSWQKAAGVAER | |
| | | YDILLLTENGILLRNTSEPATTKQHKFEDLTPG | |
| | | KKYKIQILTVSGGLFSKEAQTEGRTVPAAVTD | |
| | | LRITENSTRHLSFRWTASEGELSWYNIFLYNPD | |
| | | GNLQERAQVDPLVQSFSFQNLLQGRMYKMVI | |
| | | VTHSGELSNESFIFGRTVPASVSHLRGSNRNTT | |
| | | DSLWFNWSPASGDFDFYELILYNPNGTKKEN | |
| | | WKDKDLTEWRFQGLVPGRKYVLWVVTHSGD | |
| | | LSNKVTAESRTAPSPPSLMSFADIANTSLAITW | |
| | | KGPPDWTDYNDFELQWLPRDALTVFNPYNNR | |
| | | KSEGRIVYGLRPGRSYQFNVKTVSGDSWKTYS | |
| | | KPIFGSVRTKPDKIQNLHCRPQNSTAIACSWIPP | |
| | | DSDFDGYSIECRKMDTQEVEFSRKLEKEKSLL | |
| | | NIMMLVPHKRYLVSIKVQSAGMTSEVVEDSTI | |
| | | TMIDRPPPPPPHIRVNEKDVLISKSSINFTVNCS | |
| | | WFSDTNGAVKYFTVVVREADGSDELKPEQQH | |
| | | PLPSYLEYRHNASIRVYQTNYFASKCAENPNS | |
| | | NSKSFNIKLGAEMESLGGKCDPTQQKFCDGPL | |
| | | KPHTAYRISIRAFTQLFDEDLKEFTKPLYSDTFF | |
| | | SLPITTESEPLFGAIEGVSAGLFLIGMLVAVVAL | |
| | | LICRQKVSHGRERPSARLSIRRDRPLSVHLNLG | |
| | | QKGNRKTSCPIKINQFEGHFMKLQADSNYLLS | |
| | | KEYEELKDVGRNQSCDIALLPENRGKNRYNNI | |
| | | LPYDATRVKLSNVDDDPCSDYINASYIPGNNF | |
| | | RREYIVTQGPLPGTKDDFWKMVWEQNVHNIV | |
| | | MVTQCVEKGRVKCDHYWPADQDSLYYGDLI | |
| | | LQMLSESVLPEWTIREFKICGEEQLDAHRLIRH | |
| | | FHYTVWPDHGVPETTQSLIQFVRTVRDYINRS | |
| | | PGAGPTVVHCSAGVGRTGTFIALDRILQQLDS | |
| | | KDSVDIYGAVHDLRLHRVHMVQTECQYVYL | |
| | | HQCVRDVLRARKLRSEQENPLFPIYENVNPEY | |
| | | HRDPVYSRH | |
| PTPRC | Truncate 1- | MTMYLWLKLLAFGFAFLDTEVFVTGQSPTPSP | 240 |
| | 602 | TGLTTAKMPSVPLSSDPLPTHTTAFSPASTFER | |
| | | ENDFSETTTSLSPDNTSTQVSPDSLDNASAFNT | |
| | | TGVSSVQTPHLPTHADSQTPSAGTDTQTFSGS | |
| | | AANAKLNPTPGSNAISDVPGERSTASTFPTDPV | |
| | | SPLTTTLSLAHHSSAALPARTSNTTITANTSDA | |
| | | YLNASETTTLSPSGSAVISTTTIATTPSKPTCDE | |
| | | KYANITVDYLYNKETKLFTAKLNVNENVECG | |
| | | NNTCTNNEVHNLTECKNASVSISHNSCTAPDK | |
| | | TLILDVPPGVEKFQLHDCTQVEKADTTICLKW | |
| | | KNIETFTCDTQNITYRFQCGNMIFDNKEIKLEN | |
| | | LEPEHEYKCDSEILYNNHKFTNASKIIKTDFGS | |
| | | PGEPQIIFCRSEAAHQGVITWNPPQRSFHNFTL | |
| | | CYIKETEKDCLNLDKNLIKYDLQNLKPYTKYV | |
| | | LSLHAYIIAKVQRNGSAAMCHFTTKSAPPSQV | |
| | | WNMTVSMTSDNSMHVKCRPPRDRNGPHERY | |
| | | HLEVEAGNTLVRNESHKNCDFRVKDLQYSTD | |
| | | YTFKAYFHNGDYPGEPFILHHSTSYNSKALIAF | |
| | | LAFLIIVTSIALLVVLYKIYDLHKKRSCNLDEQ | |
| | | QELVERDDEKQLMNVEPIHADILLETYKRKIA | |
| | | DEGRLFLAEFQSIPRVFSKFPIKEARKPFNQNK | |
| | | NRYVDILPYDYNRVELSEINGDAGSNYINASYI | |
| | | DGFKEPRKYIAAQGPRDETVDDFWRMIWEQK | |
| | | ATVIVMVTRCEEGNRNKCAEYWPSMEEGTRA | |
| | | FGDVVVKINQHKRCPDYIIQKLNIVNKKEKAT | |
| | | GREVTHIQFTSWPDHGVPEDPHLLLKLRRRVN | |
| | | AFSNFFSGPIVVHCSAGVGRTGTYIGIDAMLEG | |
| | | LEAENKVDVYGYVVKLRRQRCLMVQVEAQY | |
| | | ILIHQALVEYNQFGETEVNLSELHPYLHNMKK | |
| | | RDPPSEPSPLEAEFQRLPSYRSWRTQHIGNQEE | |
| | | NKSKNRNSNVIPYDYNRVPLKHELEMSKESEH | |
| | | DSDESSDDDSDSEEPSKYINASFIMSYWKPEV | |
| | | MIAAQGPLKETIGDFWQMIFQRKVKVIVMLTE | |
| | | LKHGDQEICAQYWGEGKQTYGDIEVDLKDTD | |
| | | KSSTYTLRVFELRHSKRKDSRTVYQYQYTNW | |
| | | SVEQLPAEPKELISMIQVVKQKLPQKNSSEGN | |
| | | KHHKSTPLLIHCRDGSQQTGIFCALLNLLESAE | |
| | | TEEVVDIFQVVKALRKARPGMVSTFEQYQFLY | |
| | | DVIASTYPAQNGQVKKNNHQEDKIEFDNEVD | |
| | | KVKQDANCVNPLGAPEKLPEAKEQAEGSEPTS | |
| | | GTEGPEHSVNGPASPALNQGS | |
| PTPN6 | Truncate 1- | MVRWFHRDLSGLDAETLLKGRGVHGSFLARP | 241 |
| | 220 and | SRKNQGDFSLSVRVGDQVTHIRIQNSGDFYDL | |
| | 544-595 | YGGEKFATLTELVEYYTQQQGVLQDRDGTIIH | |
| | | LKYPLNCSDPTSERWYHGHMSGGQAETLLQA | |
| | | KGEPWTFLVRESLSQPGDFVLSVLSDQPKAGP | |
| | | GSPLRVTHIKVMCEGGRYTVGGLETFDSLTDL | |
| | | VEHFKKTGIEEASGAFVYLRQPYYATRVNAA | |
| | | DIENRVLELNKKQESEDTAKAGFWEEFESLQK | |
| | | QEVKNLHQRLEGQRPENKGKNRYKNILPFDHS | |
| | | RVILQGRDSNIPGSDYINANYIKNQLLGPDENA | |
| | | KTYIASQGCLEATVNDFWQMAWQENSRVIVM | |
| | | TTREVEKGRNKCVPYWPEVGMQRAYGPYSVT | |
| | | NCGEHDTTEYKLRTLQVSPLDNGDLIREIWHY | |
| | | QYLSWPDHGVPSEPGGVLSFLDQINQRQESLP | |
| | | HAGPIIVHCSAGIGRTGTIIVIDMLMENISTKGL | |
| | | DCDIDIQKTIQMVRAQRSGMVQTEAQYKFIYV | |
| | | AIAQFIETTKKKLEVLQSQKGQESEYGNITYPP | |
| | | AMKNAHAKASRTSSKHKEDVYENLHTKNKR | |
| | | EEKVKKQRSADKEKSKGSLKRK | |
| PTPN22 | Truncate 307- | MDQREILQKFLDEAQSKKITKEEFANEFLKLK | 242 |
| | 807 | RQSTKYKADKTYPTTVAEKPKNIKKNRYKDIL | |
| | | PYDYSRVELSLITSDEDSSYINANFIKGVYGPK | |
| | | AYIATQGPLSTTLLDFWRMIWEYSVLIIVMAC | |
| | | MEYEMGKKKCERYWAEPGEMQLEFGPFSVSC | |
| | | EAEKRKSDYIIRTLKVKFNSETRTIYQFHYKN | |
| | | WPDHDVPSSIDPILELIWDVRCYQEDDSVPICI | |
| | | HCSAGCGRTGVICAIDYTWMLLKDGIIPENFSV | |
| | | FSLIREMRTQRPSLVQTQEQYELVYNAVLELF | |
| | | KRQMDVIRDKHSGTESQAKHCIPEKNHTLQA | |
| | | DSYSPNLPKSTTKAAKMMNQQRTKMEIKESSS | |
| | | FDFRTSEISAKEELVLHPAKSSTSFDFLELNYSF | |
| | | DKNADTTMKWQTKAFPIVGEPLQKHQSLDLG | |
| | | SLLFEGCSNSKPVNAAGRYFNSKVPITRTKSTP | |
| | | FELIQQRETKEVDSKENFSYLESQPHDSCFVEM | |
| | | QAQKVMHVSSAELNYSLPYDSKHQIRNASNV | |
| | | KHHDSSALGVYSYIPLVENPYFSSWPPSGTSSK | |
| | | MSLDLPEKQDGTVFPSSLLPTSSTSLFSYYNSH | |
| | | DSLSLNSPTNISSLLNQESAVLATAPRIDDEIPP | |
| | | PLPVRTPESFIVVEEAGEFSPNVPKSLSSAVKV | |
| | | KIGTSLEWGGTSEPKKFDDSVILRPSKSVKLRS | |
| | | PKSELHQDRSSPPPPLPERTLESFFLADEDCMQ | |
| | | AQSIETYSTSYPDTMENSTSSKQTLKTPGKSFT | |
| | | RSKSLKILRNMKKSICNSCPPNKPAESVQSNNS | |
| | | SSFLNFGFANRFSKPKGPRNPPPTWNI | |
| PTPRS | Truncate 1- | MAPTWGPGMVSVVGPMGLLVVLLVGGCAAE | 243 |
| | 1343 and | EPPRFIKEPKDQIGVSGGVASFVCQATGDPKPR | |
| | 1927-1948 | VTWNKKGKKVNSQRFETIEFDESAGAVLRIQP | |
| | | LRTPRDENVYECVAQNSVGEITVHAKLTVLRE | |
| | | DQLPSGFPNIDMGPQLKVVERTRTATMLCAAS | |
| | | GNPDPEITWFKDFLPVDPSASNGRIKQLRSETF | |
| | | ESTPIRGALQIESSEETDQGKYECVATNSAGVR | |
| | | YSSPANLYVRELREVRRVAPRFSILPMSHEIMP | |
| | | GGNVNITCVAVGSPMPYVKWMQGAEDLTPE | |
| | | DDMPVGRNVLELTDVKDSANYTCVAMSSLG | |
| | | VIEAVAQITVKSLPKAPGTPMVTENTATSITIT | |
| | | WDSGNPDPVSYYVIEYKSKSQDGPYQIKEDIT | |
| | | TTRYSIGGLSPNSEYEIWVSAVNSIGQGPPSESV | |
| | | VTRTGEQAPASAPRNVQARMLSATTMIVQWE | |
| | | EPVEPNGLIRGYRVYYTMEPEHPVGNWQKHN | |
| | | VDDSLLTTVGSLLEDETYTVRVLAFTSVGDGP | |
| | | LSDPIQVKTQQGVPGQPMNLRAEARSETSITLS | |
| | | WSPPRQESIIKYELLFREGDHGREVGRTFDPTT | |
| | | SYVVEDLKPNTEYAFRLAARSPQGLGAFTPVV | |
| | | RQRTLQSKPSAPPQDVKCVSVRSTAILVSWRP | |
| | | PPPETHNGALVGYSVRYRPLGSEDPEPKEVNGI | |
| | | PPTTTQILLEALEKWTQYRITTVAHTEVGPGPE | |
| | | SSPVVVRTDEDVPSAPPRKVEAEALNATAIRV | |
| | | LWRSPAPGRQHGQIRGYQVHYVRMEGAEAR | |
| | | GPPRIKDVMLADAQWETDDTAEYEMVITNLQ | |
| | | PETAYSITVAAYTMKGDGARSKPKVVVTKGA | |
| | | VLGRPTLSVQQTPEGSLLARWEPPAGTAEDQV | |
| | | LGYRLQFGREDSTPLATLEFPPSEDRYTASGVH | |
| | | KGATYVFRLAARSRGGLGEEAAEVLSIPEDTP | |
| | | RGHPQILEAAGNASAGTVLLRWLPPVPAERNG | |
| | | AIVKYTVAVREAGALGPARETELPAAAEPGAE | |
| | | NALTLQGLKPDTAYDLQVRAHTRRGPGPFSPP | |
| | | VRYRTFLRDQVSPKNFKVKMIMKTSVLLSWE | |
| | | FPDNYNSPTPYKIQYNGLTLDVDGRTTKKLIT | |
| | | HLKPHTFYNFVLTNRGSSLGGLQQTVTAWTA | |
| | | FNLLNGKPSVAPKPDADGFIMVYLPDGQSPVP | |
| | | VQSYFIVMVPLRKSRGGQFLTPLGSPEDMDLE | |
| | | ELIQDISRLQRRSLRHSRQLEVPRPYIAARFSVL | |
| | | PPTFHPGDQKQYGGFDNRGLEPGHRYVLFVL | |
| | | AVLQKSEPTFAASPFSDPFQLDNPDPQPIVDGE | |
| | | EGLIWVIGPVLAVVFIICIVIAILLYKNKPDSKR | |
| | | KDSEPRTKCLLNNADLAPHHPKDPVEMRRINF | |
| | | QTPDSGLRSPLREPGFHFESMLSHPPIPIADMA | |
| | | EHTERLKANDSLKLSQEYESIDPGQQFTWEHS | |
| | | NLEVNKPKNRYANVIAYDHSRVILQPIEGIMGS | |
| | | DYINANYVDGYRCQNAYIATQGPLPETFGDF | |
| | | WRMVWEQRSATIVMMTRLEEKSRIKCDQYW | |
| | | PNRGTETYGFIQVTLLDTIELATFCVRTFSLHK | |
| | | NGSSEKREVRQFQFTAWPDHGVPEYPTPFLAF | |
| | | LRRVKTCNPPDAGPIVVHCSAGVGRTGCFIVID | |
| | | AMLERIKPEKTVDVYGHVTLMRSQRNYMVQT | |
| | | EDQYSFIHEALLEAVGCGNTEVPARSLYAYIQ | |
| | | KLAQVEPGEHVTGMELEFKRLANSKAHTSRFI | |
| | | SANLPCNKFKNRLVNIMPYESTRVCLQPIRGV | |
| | | EGSDYINASFIDGYRQQKAYIATQGPLAETTED | |
| | | FWRMLWENNSTIVVMLTKLREMGREKCHQY | |
| | | WPAERSARYQYFVVDPMAEYNMPQYILREFK | |
| | | VTDARDGQSRTVRQFQFTDWPEQGVPKSGEG | |
| | | FIDFIGQVHKTKEQFGQDGPISVHCSAGVGRTG | |
| | | VFITLSIVLERMRYEGVVDIFQTVKMLRTQRPA | |
| | | MVQTEDEYQFCYQAALEYLGSFDHYAT | |
| PTPRM | | MRGLGTCLATLAGLLLTAAGETFSGGCLFDEP | 244 |
| | | YSTCGYSQSEGDDFNWEQVNTLTKPTSDPWM | |
| | | PSGSFMLVNASGRPEGQRAHLLLPQLKENDTH | |
| | | CIDFHYFVSSKSNSPPGLLNVYVKVNNGPLGN | |
| | | PIWNISGDPTRTWNRAELAISTFWPNFYQVIFE | |
| | | VITSGHQGYLAIDEVKVLGHPCTRTPHFLRIQN | |
| | | VEVNAGQFATFQCSAIGRTVAGDRLWLQGID | |
| | | VRDAPLKEIKVTSSRRFIASFNVVNTTKRDAG | |
| | | KYRCMIRTEGGVGISNYAELVVKEPPVPIAPPQ | |
| | | LASVGATYLWIQLNANSINGDGPIVAREVEYC | |
| | | TASGSWNDRQPVDSTSYKIGHLDPDTEYEISV | |
| | | LLTRPGEGGTGSPGPALRTRTKCADPMRGPRK | |
| | | LEVVEVKSRQITIRWEPFGYNVTRCHSYNLTV | |
| | | HYCYQVGGQEQVREEVSWDTENSHPQHTITN | |
| | | LSPYTNVSVKLILMNPEGRKESQELIVQTDEDL | |
| | | PGAVPTESIQGSTFEEKIFLQWREPTQTYGVITL | |
| | | YEITYKAVSSFDPEIDLSNQSGRVSKLGNETHF | |
| | | LFFGLYPGTTYSFTIRASTAKGFGPPATNQFTT | |
| | | KISAPSMPAYELETPLNQTDNTVTVMLKPAHS | |
| | | RGAPVSVYQIVVEEERPRRTKKTTEILKCYPVP | |
| | | IHFQNASLLNSQYYFAAEFPADSLQAAQPFTIG | |
| | | DNKTYNGYWNTPLLPYKSYRIYFQAASRANG | |
| | | ETKIDCVQVATKGAATPKPVPEPEKQTDHTVK | |
| | | IAGVIAGILLFVIIFLGVVLVMKKRKLAKKRKE | |
| | | TMSSTRQEMTVMVNSMDKSYAEQGTNCDEA | |
| | | FSFMDTHNLNGRSVSSPSSFTMKTNTLSTSVPN | |
| | | SYYPDETHTMASDTSSLVQSHTYKKREPADVP | |
| | | YQTGQLHPAIRVADLLQHITQMKCAEGYGFK | |
| | | EEYESFFEGQSAPWDSAKKDENRMKNRYGNII | |
| | | AYDHSRVRLQTIEGDTNSDYINGNYIDGYHRP | |
| | | NHYIATQGPMQETIYDFWRMVWHENTASIIM | |
| | | VTNLVEVGRVKCCKYWPDDTEIYKDIKVTLIE | |
| | | TELLAEYVIRTFAVEKRGVHEIREIRQFHFTGW | |
| | | PDHGVPYHATGLLGFVRQVKSKSPPSAGPLVV | |
| | | HCSAGAGRTGCFIVIDIMLDMAEREGVVDIYN | |
| | | CVRELRSRRVNMVQTEEQYVFIHDAILEACLC | |
| | | GDTSVPASQVRSLYYDMNKLDPQTNSSQIKEE | |
| | | FRTLNMVTPTLRVEDCSIALLPRNHEKNRCMD | |
| | | ILPPDRCLPFLITIDGESSNYINAALMDSYKQPS | |
| | | AFIVTQHPLPNTVKDFWRLVLDYHCTSVVML | |
| | | NDVDPAQLCPQYWPENGVHRHGPIQVEFVSA | |
| | | DLEEDIISRIFRIYNAARPQDGYRMVQQFQFLG | |
| | | WPMYRDTPVSKRSFLKLIRQVDKWQEEYNGG | |
| | | EGRTVVHCLNGGGRSGTFCAISIVCEMLRHQR | |
| | | TVDVFHAVKTLRNNKPNMVDLLDQYKFCYE | |
| | | VALEYLNSG | |
| PTPRZ | Truncate 1- | MRILKRFLACIQLLCVCRLDWANGYYRQQRK | 245 |
| | 1678 | LVEEIGWSYTGALNQKNWGKKYPTCNSPKQS | |
| | | PINIDEDLTQVNVNLKKLKFQGWDKTSLENTFI | |
| | | HNTGKTVEINLTNDYRVSGGVSEMVFKASKIT | |
| | | FHWGKCNMSSDGSEHSLEGQKFPLEMQIYCF | |
| | | DADRESSFEEAVKGKGKLRALSILFEVGTEENL | |
| | | DFKAIIDGVESVSRFGKQAALDPFILLNLLPNST | |
| | | DKYYIYNGSLTSPPCTDTVDWIVFKDTVSISES | |
| | | QLAVFCEVLTMQQSGYVMLMDYLQNNFREQ | |
| | | QYKFSRQVFSSYTGKEEIHEAVCSSEPENVQA | |
| | | DPENYTSLLVTWERPRVVYDTMIEKFAVLYQ | |
| | | QLDGEDQTKHEFLTDGYQDLGAILNNLLPNM | |
| | | SYVLQIVAICTNGLYGKYSDQLIVDMPTDNPE | |
| | | LDLFPELIGTEEIIKEEEEGKDIEEGAIVNPGRDS | |
| | | ATNQIRKKEPQISTTTHYNRIGTKYNEAKTNRS | |
| | | PTRGSEFSGKGDVPNTSLNSTSQPVTKLATEK | |
| | | DISLTSQTVTELPPHTVEGTSASLNDGSKTVLR | |
| | | SPHMNLSGTAESLNTVSITEYEEESLLTSFKLD | |
| | | TGAEDSSGSSPATSAIPFISENISQGYIFSSENPE | |
| | | TITYDVLIPESARNASEDSTSSGSEESLKDPSME | |
| | | GNVWFPSSTDITAQPDVGSGRESFLQTNYTEIR | |
| | | VDESEKTTKSFSAGPVMSQGPSVTDLEMPHYS | |
| | | TFAYFPTEVTPHAFTPSSRQQDLVSTVNVVYS | |
| | | QTTQPVYNGETPLQPSYSSEVFPLVTPLLLDNQ | |
| | | ILNTTPAASSSDSALHATPVFPSVDVSFESILSS | |
| | | YDGAPLLPFSSASFSSELFRHLHTVSQILPQVTS | |
| | | ATESDKVPLHASLPVAGGDLLLEPSLAQYSDV | |
| | | LSTTHAASETLEFGSESGVLYKTLMFSQVEPPS | |
| | | SDAMMHARSSGPEPSYALSDNEGSQHIFTVSY | |
| | | SSAIPVHDSVGVTYQGSLFSGPSHIPIPKSSLITP | |
| | | TASLLQPTHALSGDGEWSGASSDSEFLLPDTD | |
| | | GLTALNISSPVSVAEFTYTTSVFGDDNKALSKS | |
| | | EIIYGNETELQIPSFNEMVYPSESTVMPNMYDN | |
| | | VNKLNASLQETSVSISSTKGMFPGSLAHTTTK | |
| | | VFDHEISQVPENNFSVQPTHTVSQASGDTSLKP | |
| | | VLSANSEPASSDPASSEMLSPSTQLLFYETSAS | |
| | | FSTEVLLQPSFQASDVDTLLKTVLPAVPSDPIL | |
| | | VETPKVDKISSTMLHLIVSNSASSENMLHSTSV | |
| | | PVFDVSPTSHMHSASLQGLTISYASEKYEPVLL | |
| | | KSESSHQVVPSLYSNDELFQTANLEINQAHPPK | |
| | | GRHVFATPVLSIDEPLNTLINKLIHSDEILTSTK | |
| | | SSVTGKVFAGIPTVASDTFVSTDHSVPIGNGHV | |
| | | AITAVSPHRDGSVTSTKLLFPSKATSELSHSAK | |
| | | SDAGLVGGGEDGDTDDDGDDDDDDRGSDGL | |
| | | SIHKCMSCSSYRESQEKVMNDSDTHENSLMD | |
| | | QNNPISYSLSENSEEDNRVTSVSSDSQTGMDRS | |
| | | PGKSPSANGLSQKHNDGKEENDIQTGSALLPL | |
| | | SPESKAWAVLTSDEESGSGQGTSDSLNENETS | |
| | | TDFSFADTNEKDADGILAAGDSEITPGFPQSPT | |
| | | SSVTSENSEVFHVSEAEASNSSHESRIGLAEGL | |
| | | ESEKKAVIPLVIVSALTFICLVVLVGILIYWRKC | |
| | | FQTAHFYLEDSTSPRVISTPPTPIFPISDDVGAIPI | |
| | | KHFPKHVADLHASSGFTEEFETLKEFYQEVQS | |
| | | CTVDLGITADSSNHPDNKHKNRYINIVAYDHS | |
| | | RVKLAQLAEKDGKLTDYINANYVDGYNRPKA | |
| | | YIAAQGPLKSTAEDFWRMIWEHNVEVIVMITN | |
| | | LVEKGRRKCDQYWPADGSEEYGNFLVTQKSV | |
| | | QVLAYYTVRNFTLRNTKIKKGSQKGRPSGRV | |
| | | VTQYHYTQWPDMGVPEYSLPVLTFVRKAAYA | |
| | | KRHAVGPVVVHCSAGVGRTGTYIVLDSMLQQ | |
| | | IQHEGTVNIFGFLKHIRSQRNYLVQTEEQYVFI | |
| | | HDTLVEAILSKETEVLDSHIHAYVNALLIPGPA | |
| | | GKTKLEKQFQLLSQSNIQQSDYSAALKQCNRE | |
| | | KNRTSSIIPVERSRVGISSLSGEGTDYINASYIM | |
| | | GYYQSNEFIITQHPLLHTIKDFWRMIWDHNAQ | |
| | | LVVMIPDGQNMAEDEFVYWPNKDEPINCESF | |
| | | KVTLMAEEHKCLSNEEKLIIQDFILEATQDDYV | |
| | | LEVRHFQCPKWPNPDSPISKTFELISVIKEEAAN | |
| | | RDGPMIVHDEHGGVTAGTFCALTTLMHQLEK | |
| | | ENSVDVYQVAKMINLMRPGVFADIEQYQFLY | |
| | | KVILSLVSTRQEENPSTSLDSNGAALPDGNIAE | |
| | | SLESLVT | |
| Src | Truncate 1- | MGSNKSKPKDASQRRRSLEPAENVHGAGGGA | 246 |
| | 250 | FPASQTPSKPASADGHRGPSAAFAPAAAEPKL | |
| | | FGGFNSSDTVTSPQRAGPLAGGVTTFVALYDY | |
| | | ESRTETDLSFKKGERLQIVNNTEGDWWLAHSL | |
| | | STGQTGYIPSNYVAPSDSIQAEEWYFGKITRRE | |
| | | SERLLLNAENPRGTFLVRESETTKGAYCLSVS | |
| | | DFDNAKGLNVKHYKIRKLDSGGFYITSRTQFN | |
| | | SLQQLVAYYSKHADGLCHRLTTVCPTSKPQTQ | |
| | | GLAKDAWEIPRESLRLEVKLGQGCFGEVWMG | |
| | | TWNGTTRVAIKTLKPGTMSPEAFLQEAQVMK | |
| | | KLRHEKLVQLYAVVSEEPIYIVTEYMSKGSLL | |
| | | DFLKGETGKYLRLPQLVDMAAQIASGMAYVE | |
| | | RMNYVHRDLRAANILVGENLVCKVADFGLAR | |
| | | LIEDNEYTARQGAKFPIKWTAPEAALYGRFTIK | |
| | | SDVWSFGILLTELTTKGRVPYPGMVNREVLDQ | |
| | | VERGYRMPCPPECPESLHDLMCQCWRKEPEE | |
| | | RPTFEYLQAFLEDYFTSTEPQYQPGENL | |
| Lck | Truncate 1- | MGCGCSSHPEDDWMENIDVCENCHYPIVPLD | 247 |
| | 206 and | GKGTLLIRNGSEVRDPLVTYEGSNPPASPLQD | |
| | 497-509 | NLVIALHSYEPSHDGDLGFEKGEQLRILEQSGE | |
| | | WWKAQSLTTGQEGFIPFNFVAKANSLEPEPWF | |
| | | FKNLSRKDAERQLLAPGNTHGSFLIRESESTAG | |
| | | SFSLSVRDFDQNQGEVVKHYKIRNLDNGGFYI | |
| | | SPRITFPGLHELVRHYTNASDGLCTRLSRPCQT | |
| | | QKPQKPWWEDEWEVPRETLKLVERLGAGQF | |
| | | GEVWMGYYNGHTKVAVKSLKQGSMSPDAFL | |
| | | AEANLMKQLQHQRLVRLYAVVTQEPIYIITEY | |
| | | MENGSLVDFLKTPSGIKLTINKLLDMAAQIAE | |
| | | GMAFIEERNYIHRDLRAANILVSDTLSCKIADF | |
| | | GLARLIEDNEYTAREGAKFPIKWTAPEAINYG | |
| | | TFTIKSDVWSFGILLTEIVTHGRIPYPGMTNPEV | |
| | | IQNLERGYRMVRPDNCPEELYQLMRLCWKER | |
| | | PEDRPTFDYLRSVLEDFFTATEGQYQPQP | |
| Fyn | Truncate 1- | MGCVQCKDKEATKLTEERDGSLNQSSGYRYG | 248 |
| | 248 | TDPTPQHYPSFGVTSIPNYNNFHAAGGQGLTV | |
| | | FGGVNSSSHTGTLRTRGGTGVTLFVALYDYEA | |
| | | RTEDDLSFHKGEKFQILNSSEGDWWEARSLTT | |
| | | GETGYIPSNYVAPVDSIQAEEWYFGKLGRKDA | |
| | | ERQLLSFGNPRGTFLIRESETTKGAYSLSIRDW | |
| | | DDMKGDHVKHYKIRKLDNGGYYITTRAQFET | |
| | | LQQLVQHYSERAAGLCCRLVVPCHKGMPRLT | |
| | | DLSVKTKDVWEIPRESLQLIKRLGNGQFGEVW | |
| | | MGTWNGNTKVAIKTLKPGTMSPESFLEEAQIM | |
| | | KKLKHDKLVQLYAVVSEEPIYIVTEYMNKGSL | |
| | | LDFLKDGEGRALKLPNLVDMAAQVAAGMAY | |
| | | IERMNYIHRDLRSANILVGNGLICKIADFGLAR | |
| | | LIEDNEYTARQGAKFPIKWTAPEAALYGRFTIK | |
| | | SDVWSFGILLTELVTKGRVPYPGMNNREVLEQ | |
| | | VERGYRMPCPQDCPISLHELMIHCWKKDPEER | |
| | | PTFEYLQSFLEDYFTATEPQYQPGENL | |
| Yes | Truncate 1- | MGCIKSKENKSPAIKYRPENTPEPVSTSVSHYG | 249 |
| | 257 | AEPTTVSPCPSSSAKGTAVNFSSLSMTPFGGSS | |
| | | GVTPFGGASSSFSVVPSSYPAGLTGGVTIFVAL | |
| | | YDYEARTTEDLSFKKGERFQIINNTEGDWWEA | |
| | | RSIATGKNGYIPSNYVAPADSIQAEEWYFGKM | |
| | | GRKDAERLLLNPGNQRGIFLVRESETTKGAYS | |
| | | LSIRDWDEIRGDNVKHYKIRKLDNGGYYITTR | |
| | | AQFDTLQKLVKHYTEHADGLCHKLTTVCPTV | |
| | | KPQTQGLAKDAWEIPRESLRLEVKLGQGCFGE | |
| | | VWMGTWNGTTKVAIKTLKPGTMMPEAFLQE | |
| | | AQIMKKLRHDKLVPLYAVVSEEPIYIVTEFMS | |
| | | KGSLLDFLKEGDGKYLKLPQLVDMAAQIADG | |
| | | MAYIERMNYIHRDLRAANILVGENLVCKIADF | |
| | | GLARLIEDNEYTARQGAKFPIKWTAPEAALYG | |
| | | RFTIKSDVWSFGILQTELVTKGRVPYPGMVNR | |
| | | EVLEQVERGYRMPCPQGCPESLHELMNLCWK | |
| | | KDPDERPTFEYIQSFLEDYFTATEPQYQPGENL | |
| Epha2 | Truncate 1- | MELQAARACFALLWGCALAAAAAAQGKEVV | 250 |
| | 503 and | LLDFAAAGGELGWLTHPYGKGWDLMQNIMN | |
| | 923-976 | DMPIYMYSVCNVMSGDQDNWLRTNWVYRGE | |
| | | AERIFIELKFTVRDCNSFPGGASSCKETFNLYY | |
| | | AESDLDYGTNFQKRLFTKIDTIAPDEITVSSDFE | |
| | | ARHVKLNVEERSVGPLTRKGFYLAFQDIGACV | |
| | | ALLSVRVYYKKCPELLQGLAHFPETIAGSDAP | |
| | | SLATVAGTCVDHAVVPPGGEEPRMHCAVDGE | |
| | | WLVPIGQCLCQAGYEKVEDACQACSPGFFKFE | |
| | | ASESPCLECPEHTLPSPEGATSCECEEGFFRAP | |
| | | QDPASMPCTRPPSAPHYLTAVGMGAKVELRW | |
| | | TPPQDSGGREDIVYSVTCEQCWPESGECGPCE | |
| | | ASVRYSEPPHGLTRTSVTVSDLEPHMNYTFTV | |
| | | EARNGVSGLVTSRSFRTASVSINQTEPPKVRLE | |
| | | GRSTTSLSVSWSIPPPQQSRVWKYEVTYRKKG | |
| | | DSNSYNVRRTEGFSVTLDDLAPDTTYLVQVQ | |
| | | ALTQEGQGAGSKVHEFQTLSPEGSGNLAVIGG | |
| | | VAVGVVLLLVLAGVGFFIHRRRKNQRARQSPE | |
| | | DVYFSKSEQLKPLKTYVDPHTYEDPNQAVLKF | |
| | | TTEIHPSCVTRQKVIGAGEFGEVYKGMLKTSS | |
| | | GKKEVPVAIKTLKAGYTEKQRVDFLGEAGIM | |
| | | GQFSHHNIIRLEGVISKYKPMMIITEYMENGAL | |
| | | DKFLREKDGEFSVLQLVGMLRGIAAGMKYLA | |
| | | NMNYVHRDLAARNILVNSNLVCKVSDFGLSR | |
| | | VLEDDPEATYTTSGGKIPIRWTAPEAISYRKFT | |
| | | SASDVWSFGIVMWEVMTYGERPYWELSNHE | |
| | | VMKAINDGFRLPTPMDCPSAIYQLMMQCWQQ | |
| | | ERARRPKFADIVSILDKLIRAPDSLKTLADFDP | |
| | | RVSIRLPSTSGSEGVPFRTVSEWLESIKMQQYT | |
| | | EHFMAAGYTAIEKVVQMINDDIKRIGVRLPG | |
| | | HQKRIAYSLLGLKDQVNTVGIPI | |
| BTK | Truncate 1- | MAAVILESIFLKRSQQKKKTSPLNFKKRLFLLT | 251 |
| | 381 | VHKLSYYEYDFERGRRGSKKGSIDVEKITCVE | |
| | | TVVPEKNPPPERQIPRRGEESSEMEQISIIERFPY | |
| | | PFQVVYDEGPLYVFSPTEELRKRWIHQLKNVI | |
| | | RYNSDLVQKYHPCFWIDGQYLCCSQTAKNAM | |
| | | GCQILENRNGSLKPGSSHRKTKKPLPPTPEEDQ | |
| | | ILKKPLPPEPAAAPVSTSELKKVVALYDYMPM | |
| | | NANDLQLRKGDEYFILEESNLPWWRARDKNG | |
| | | QEGYIPSNYVTEAEDSIEMYEWYSKHMTRSQA | |
| | | EQLLKQEGKEGGFIVRDSSKAGKYTVSVFAKS | |
| | | TGDPQGVIRHYVVCSTPQSQYYLAEKHLFSTIP | |
| | | ELINYHQHNSAGLISRLKYPVSQQNKNAPSTA | |
| | | GLGYGSWEIDPKDLTFLKELGTGQFGVVKYG | |
| | | KWRGQYDVAIKMIKEGSMSEDEFIEEAKVMM | |
| | | NLSHEKLVQLYGVCTKQRPIFIITEYMANGCLL | |
| | | NYLREMRHRFQTQQLLEMCKDVCEAMEYLES | |
| | | KQFLHRDLAARNCLVNDQGVVKVSDFGLSRY | |
| | | VLDDEYTSSVGSKFPVRWSPPEVLMYSKFSSK | |
| | | SDIWAFGVLMWEIYSLGKMPYERFTNSETAEH | |
| | | IAQGLRLYRPHLASEKVYTIMYSCWHEKADER | |
| | | PTFKILLSNILDVMDEES | |

TABLE 28
Components of B2H Systems

| | | | SEQ | | SEQ |
|---|---|---|---|---|---|
| | | | ID | Amino | ID |
| Component | Name | DNA | NO. | Acid | NO. |
| Protein | PTP1B₃₂₁ | ATGGAGATGGAAA | 252 | MEMEKE | 253 |
| tyrosine | | AGGAGTTCGAGCA | | FEQIDK | |
| phospha- | | GATCGACAAGTCCG | | SGSWAA | |
| tase | | GGAGCTGGGCGGC | | IYQDIR | |
| 1B | | CATTTACCAGGATA | | HEASDF | |
| catalytic | | TCCGACATGAAGCC | | PCRVAK | |
| domain | | AGTGACTTCCCATG | | LPKNKN | |
| | | TAGAGTGGCCAAG | | RNRRDY | |
| | | CTTCCTAAGAACAA | | VSPFDH | |
| | | AAACCGAAATAGG | | SRIKLH | |
| | | TACAGAGACGTCA | | QEDNDY | |
| | | GTCCCTTTGACCAT | | INASLI | |
| | | AGTCGGATTAAACT | | KMEEAQ | |
| | | ACATCAAGAAGAT | | RSYILT | |
| | | AATGACTATATCAA | | QGPLPN | |
| | | CGCTAGTTTGATAA | | TCGHFW | |
| | | AAATGGAAGAAGC | | EMVWEQ | |
| | | CCAAAGGAGTTAC | | KSRGVV | |
| | | ATTCTTACCCAGGG | | MLNRVM | |
| | | CCCTTTGCCTAACA | | EKGSLK | |
| | | CATGCGGTCACTTT | | CAQYWP | |
| | | TGGGAGATGGTGTG | | QKEEKE | |
| | | GGAGCAGAAAAGC | | MIFEDT | |
| | | AGGGGTGTCGTCAT | | NLKLTL | |
| | | GCTCAACAGAGTG | | ISEDIK | |
| | | ATGGAGAAAGGTT | | SYYTVR | |
| | | CGTTAAAATGCGCA | | QLELEN | |
| | | CAATACTGGCCACA | | LTTQET | |
| | | AAAAGAAGAAAAA | | REILHF | |
| | | GAGATGATCTTTGA | | HYTTWP | |
| | | AGACACAAATTTGA | | DFGVPE | |
| | | AATTAACATTGATC | | SPASFL | |
| | | TCTGAAGATATCAA | | NFLFKV | |
| | | GTCATATTATACAG | | RESGSL | |
| | | TGCGACAGCTAGA | | SPEHGP | |
| | | ATTGGAAAACCTTA | | VVVHCS | |
| | | CAACCCAAGAAAC | | AGIGRS | |
| | | TCGAGAGATCTTAC | | GTFCLA | |
| | | ATTTCCACTATACC | | DTCLLL | |
| | | ACATGGCCTGACTT | | MDKRKD | |
| | | TGGAGTCCCTGAAT | | PSSVDI | |
| | | CACCAGCCTCATTC | | KKVLLE | |
| | | TTGAACTTTCTTTT | | MRKFRM | |
| | | CAAAGTCCGAGAGT | | GLIQTA | |
| | | CAGGGTCACTCAGC | | DQLRFS | |
| | | CCGGAGCACGGGC | | YLAVIE | |
| | | CCGTTGTGGTGCAC | | GAKFIM | |
| | | TGCAGTGCAGGCAT | | GDSSVQ | |
| | | CGGCAGGTCTGGA | | DQWKEL | |
| | | ACCTTCTGTCTGGC | | SHEDLE | |
| | | TGATACCTGCCTCT | | PPPEHI | |
| | | TGCTGATGGACAAG | | PPPPRP | |
| | | AGGAAAGACCCTTC | | PKRILE | |
| | | TTCCGTTGATATCA | | PHN* | |
| | | AGAAAGTGCTGTTA | | | |
| | | GAAATGAGGAAGT | | | |
| | | TTCGGATGGGGCTG | | | |
| | | ATCCAGACAGCCG | | | |
| | | ACCAGCTGCGCTTC | | | |
| | | TCCTACCTGGCTGT | | | |
| | | GATCGAAGGTGCC | | | |
| | | AAATTCATCATGGG | | | |
| | | GGACTCTTCCGTGC | | | |
| | | AGGATCAGTGGAA | | | |
| | | GGAGCTTTCCCACG | | | |
| | | AGGACCTGGAGCC | | | |
| | | CCCACCCGAGCATA | | | |
| | | TCCCCCCACCTCCC | | | |
| | | CGGCCACCCAAAC | | | |
| | | GAATCCTGGAGCCA | | | |
| | | CACAATTGA | | | |
| Protein | PTP1B₄₀₅ | ATGGAGATGGAAA | 254 | MEMEKE | 255 |
| tyrosine | | AGGAGTTCGAGCA | | FEQIDK | |
| phospha- | | GATCGACAAGTCCG | | SGSWAA | |
| tase | | GGAGCTGGGCGGC | | IYQDIR | |
| 1B 1-405 | | CATTTACCAGGATA | | HEASDF | |
| | | TCCGACATGAAGCC | | PCRVAK | |
| | | AGTGACTTCCCATG | | LPKNKN | |
| | | TAGAGTGGCCAAG | | RNRYRD | |
| | | CTTCCTAAGAACAA | | VSPFDH | |
| | | AAACCGAAATAGG | | SRIKLH | |
| | | TACAGAGACGTCA | | QEDNDY | |
| | | GTCCCTTTGACCAT | | INASLI | |
| | | AGTCGGATTAAACT | | KMEEAQ | |
| | | ACATCAAGAAGAT | | RSYILT | |
| | | AATGACTATATCAA | | QGPLPN | |
| | | CGCTAGTTTGATAA | | TCGHFW | |
| | | AAATGGAAGAAGC | | EMVWEQ | |
| | | CCAAAGGAGTTAC | | KSRGVV | |
| | | ATTCTTACCCAGGG | | MLNRVM | |
| | | CCCTTTGCCTAACA | | EKGSLK | |
| | | CATGCGGTCACTTT | | CAQYWP | |
| | | TGGGAGATGGTGTG | | QKEEKE | |
| | | GGAGCAGAAAAGC | | MIFEDT | |
| | | AGGGGTGTCGTCAT | | NLKLTL | |
| | | GCTCAACAGAGTG | | ISEDIK | |
| | | ATGGAGAAAGGTT | | SYYTVR | |
| | | CGTTAAAATGCGCA | | QLELEN | |
| | | CAATACTGGCCACA | | LTTQET | |
| | | AAAAGAAGAAAAA | | REILHF | |
| | | GAGATGATCTTTGA | | HYTTWP | |
| | | AGACACAAATTTGA | | DFGVPE | |
| | | AATTAACATTGATC | | SPASFL | |
| | | TCTGAAGATATCAA | | NFLFKV | |
| | | GTCATATTATACAG | | RESGSL | |
| | | TGCGACAGCTAGA | | SPEHGP | |
| | | ATTGGAAAACCTTA | | VVVHCS | |
| | | CAACCCAAGAAAC | | AGIGRS | |
| | | TCGAGAGATCTTAC | | GTFCLA | |
| | | ATTTCCACTATACC | | DTCLLL | |
| | | ACATGGCCTGACTT | | MDKRKD | |
| | | TGGAGTCCCTGAAT | | PSSVDI | |
| | | CACCAGCCTCATTC | | KKVLLE | |
| | | TTGAACTTTCTTTT | | MRKFRM | |
| | | CAAAGTCCGAGAGT | | GLIQTA | |
| | | CAGGGTCACTCAGC | | DQLRFS | |
| | | CCGGAGCACGGGC | | YLAVIE | |
| | | CCGTTGTGGTGCAC | | GAKFIM | |
| | | TGCAGTGCAGGCAT | | GDSSVQ | |
| | | CGGCAGGTCTGGA | | DQWKEL | |
| | | ACCTTCTGTCTGGC | | SHEDLE | |
| | | TGATACCTGCCTCT | | PPPEHI | |
| | | TGCTGATGGACAAG | | PPPPRP | |
| | | AGGAAAGACCCTTC | | PKRILE | |
| | | TTCCGTTGATATCA | | PHNGKC | |
| | | AGAAAGTGCTGTTA | | REFFPN | |
| | | GAAATGAGGAAGT | | HQWVKE | |
| | | TTCGGATGGGGCTG | | ETQEDK | |
| | | ATCCAGACAGCCG | | DCPIKE | |
| | | ACCAGCTGCGCTTC | | EKGSPL | |
| | | TCCTACCTGGCTGT | | NAAPYG | |
| | | GATCGAAGGTGCC | | IESMSQ | |
| | | AAATTCATCATGGG | | DTEVRS | |
| | | GGACTCTTCCGTGC | | RVVGGS | |
| | | AGGATCAGTGGAA | | LRGAQA | |
| | | GGAGCTTTCCCACG | | ASPAKG | |
| | | AGGACCTGGAGCC | | EPSLPE | |
| | | CCCACCCGAGCATA | | KDEDHA | |
| | | TCCCCCCACCTCCC | | LSY* | |
| | | CGGCCACCCAAAC | | | |
| | | GAATCCTGGAGCCA | | | |
| | | CACAATGGGAAAT | | | |
| | | GCAGGGAGTTCTTC | | | |
| | | CCAAATCACCAGTG | | | |
| | | GGTGAAGGAAGAG | | | |
| | | ACCCAGGAGGATA | | | |
| | | AAGACTGCCCCATC | | | |
| | | AAGGAAGAAAAAG | | | |
| | | GAAGCCCCTTAAAT | | | |
| | | GCCGCACCCTACGG | | | |
| | | CATCGAAAGCATG | | | |
| | | AGTCAAGACACTG | | | |
| | | AAGTTAGAAGTCG | | | |
| | | GGTCGTGGGGGGA | | | |
| | | AGTCTTCGAGGTGC | | | |
| | | CCAGGCTGCCTCCC | | | |
| | | CAGCCAAAGGGGA | | | |
| | | GCCGTCACTGCCCG | | | |
| | | AGAAGGACGAGGA | | | |
| | | CCATGCACTGAGTT | | | |
| | | ACTAA | | | |
| T-cell | TCPTP₃₁₇ | ATGCCCACCACCAT | 256 | MPTTIE | 257 |
| protein | | CGAGCGGGAGTTC | | REFEEL | |
| tyrosine | | GAAGAGTTGGATA | | DTQRRW | |
| phospha- | | CTCAGCGTCGCTGG | | QPLYLE | |
| tase | | CAGCCGCTGTACTT | | IRNESH | |
| catalytic | | GGAAATTCGAAAT | | DYPHRV | |
| domain | | GAGTCCCATGACTA | | AKFPEN | |
| | | TCCTCATAGAGTGG | | RNRNRY | |
| | | CCAAGTTTCCAGAA | | RDVSPY | |
| | | AACAGAAATCGAA | | DHSRVK | |
| | | ACAGATACAGAGA | | LQNAEN | |
| | | TGTAAGCCCATATG | | DYINAS | |
| | | ATCACAGTCGTGTT | | LVDIEE | |
| | | AAACTGCAAAATG | | AQRSYI | |
| | | CTGAGAATGATTAT | | LTQGPL | |
| | | ATTAATGCCAGTTT | | PNTCCH | |
| | | AGTTGACATAGAA | | FWLMVW | |
| | | GAGGCACAAAGGA | | QQKTKA | |
| | | GTTACATCTTAACA | | VVMLNR | |
| | | CAGGGTCCACTTCC | | IVEKES | |
| | | TAACACATGCTGCC | | VKCAQY | |
| | | ATTTCTGGCTTATG | | WPTDDQ | |
| | | GTTTGGCAGCAGAA | | EMLFKE | |
| | | GACCAAAGCAGTT | | TGFSVK | |
| | | GTCATGCTGAACCG | | LLSEDV | |
| | | CATTGTGGAGAAA | | KSYYTV | |
| | | GAATCGGTTAAATG | | HLLQLE | |
| | | TGCACAGTACTGGC | | NINSGE | |
| | | CAACAGATGACCA | | TRTISH | |
| | | AGAGATGCTGTTTA | | FHYTTW | |
| | | AAGAAACAGGATT | | PDFGVP | |
| | | CAGTGTGAAGCTCT | | ESPASF | |
| | | TGTCAGAAGATGTG | | LNFLFK | |
| | | AAGTCGTATTATAC | | VRESGS | |
| | | AGTACATCTACTAC | | LNPDHG | |
| | | AATTAGAAAATATC | | PAVIHC | |
| | | AATAGTGGTGAAA | | SAGIGR | |
| | | CCAGAACAATATCT | | SGTFSL | |
| | | CACTTTCATTATAC | | VDTCLV | |
| | | TACCTGGCCAGATT | | LMEKGD | |
| | | TTGGAGTCCCTGAA | | DINIKQ | |
| | | TCACCAGCTTCATT | | VLLNMR | |
| | | TCTCAATTTCTTGT | | KYRMGL | |
| | | TTAAAGTGAGAGAA | | IQTPDQ | |
| | | TCTGGCTCCTTGAA | | LRFSYM | |
| | | CCCTGACCATGGGC | | AIIEGA | |
| | | CTGCGGTGATCCAC | | KCIKGD | |
| | | TGTAGTGCAGGCAT | | SSIQKR | |
| | | TGGGCGCTCTGGCA | | WKELSK | |
| | | CCTTCTCTCTGGTA | | EDLSPA | |
| | | GACACTTGTCTTGT | | FDHSPN | |
| | | TTTGATGGAAAAAG | | KIMTEK | |
| | | GAGATGATATTAAC | | YNGNR* | |
| | | ATAAAACAAGTGTT | | | |
| | | ACTGAACATGAGA | | | |
| | | AAATACCGAATGG | | | |
| | | GTCTTATTCAGACC | | | |
| | | CCAGATCAACTGAG | | | |
| | | ATTCTCATACATGG | | | |
| | | CTATAATAGAAGG | | | |
| | | AGCAAAATGTATA | | | |
| | | AAGGGAGATTCTA | | | |
| | | GTATACAGAAACG | | | |
| | | ATGGAAAGAACTTT | | | |
| | | CTAAGGAAGACTTA | | | |
| | | TCTCCTGCCTTTGA | | | |
| | | TCATTCACCAAACA | | | |
| | | AAATAATGACTGA | | | |
| | | AAAATACAATGGG | | | |
| | | AACAGATAA | | | |
| T-cell | TCPTP₃₈₇ | ATGCCCACCACCAT | 258 | MPTTIE | 259 |
| protein | | CGAGCGGGAGTTC | | REFEEL | |
| tyrosine | | GAAGAGTTGGATA | | DTQRRW | |
| phospha- | | CTCAGCGTCGCTGG | | QPLYLE | |
| tase | | CAGCCGCTGTACTT | | IRNESH | |
| 1-387 | | GGAAATTCGAAAT | | DYPHRV | |
| | | GAGTCCCATGACTA | | AKFPEN | |
| | | TCCTCATAGAGTGG | | RNRNRY | |
| | | CCAAGTTTCCAGAA | | RDVSPY | |
| | | AACAGAAATCGAA | | DHSRVK | |
| | | ACAGATACAGAGA | | LQNAEN | |
| | | TGTAAGCCCATAcG | | DYINAS | |
| | | ATCACAGTCGTGTT | | LVDIEE | |
| | | AAACTGCAAAATG | | AQRSYI | |
| | | CTGAGAATGATTAT | | LTQGPL | |
| | | ATTAATGCCAGTTT | | PNTCCH | |
| | | AGTTGACATAGAA | | FWLMVW | |
| | | GAGGCACAAAGGA | | QQKTKA | |
| | | GTTACATCTTAACA | | VVMLNR | |
| | | CAGGGTCCACTTCC | | IVEKES | |
| | | TAACACATGCTGCC | | VKCAQY | |
| | | ATTTCTGGCTTATG | | WPTDDQ | |
| | | GTTTGGCAGCAGAA | | EMLFKE | |
| | | GACCAAAGCAGTT | | TGFSVK | |
| | | GTCATGCTGAACCG | | LLSEDV | |
| | | CATTGTGGAGAAA | | KSYYTV | |
| | | GAATCGGTTAAATG | | HLLQLE | |
| | | TGCACAGTACTGGC | | NINSGE | |
| | | CAACAGATGACCA | | TRTISH | |
| | | AGAGATGCTGTTTA | | FHYTTW | |
| | | AAGAAACAGGATT | | PDFGVP | |
| | | CAGTGTGAAGCTCT | | ESPASF | |
| | | TGTCAGAAGATGTG | | LNFLFK | |
| | | AAGTCGTATTATAC | | VRESGS | |
| | | AGTACATCTACTAC | | LNPDHG | |
| | | AATTAGAAAATATC | | PAVIHC | |
| | | AATAGTGGTGAAA | | SAGIGR | |
| | | CCAGAACAATATCT | | SGTFSL | |
| | | CACTTTCATTATAC | | VDTCLV | |
| | | TACCTGGCCAGATT | | LMEKGD | |
| | | TTGGAGTCCCTGAA | | DINIKQ | |
| | | TCACCAGCTTCATT | | VLLNMR | |
| | | TCTCAATTTCTTGT | | KYRMGL | |
| | | TTAAAGTGAGAGAA | | IQTPDQ | |
| | | TCTGGCTCCTTGAA | | LRFSYM | |
| | | CCCTGACCATGGGC | | AIIEGA | |
| | | CTGCGGTGATCCAC | | KCIKGD | |
| | | TGTAGTGCAGGCAT | | SSIQKR | |
| | | TGGGCGCTCTGGCA | | WKELSK | |
| | | CCTTCTCTCTGGTA | | EDLSPA | |
| | | GACACTTGTCTTGT | | FDHSPN | |
| | | TTTGATGGAAAAAG | | KIMTEK | |
| | | GAGATGATATTAAC | | YNGNRI | |
| | | ATAAAACAAGTGTT | | GLEEEK | |
| | | ACTGAACATGAGA | | LTGDRC | |
| | | AAATACCGAATGG | | TGLSSK | |
| | | GTCTTATTCAGACC | | MQDTME | |
| | | CCAGATCAACTGAG | | ENSESA | |
| | | ATTCTCATACATGG | | LRKRIR | |
| | | CTATAATAGAAGG | | EDRKAT | |
| | | AGCAAAATGTATA | | TAQKVQ | |
| | | AAGGGAGATTCTA | | QMKQRL | |
| | | GTATACAGAAACG | | NENERK | |
| | | ATGGAAAGAACTTT | | RKRPRL | |
| | | CTAAGGAAGACTTA | | TDT* | |
| | | TCTCCTGCCTTTGA | | | |
| | | TCATTCACCAAACA | | | |
| | | AAATAATGACTGA | | | |
| | | AAAATACAATGGG | | | |
| | | AACAGAATTGGACT | | | |
| | | TGAAGAGGAAAAA | | | |
| | | CTGACCGGGGACA | | | |
| | | GATGCACCGGACTG | | | |
| | | TCGTCGAAGATGCA | | | |
| | | AGATACTATGGAA | | | |
| | | GAGAATAGTGAAT | | | |
| | | CTGCCTTACGCAAA | | | |
| | | CGTATTAGAGAAG | | | |
| | | ATCGCAAGGCCACT | | | |
| | | ACCGCGCAGAAAG | | | |
| | | TCCAACAAATGAA | | | |
| | | ACAACGGCTGAAC | | | |
| | | GAAAACGAGAGAA | | | |
| | | AAAGAAAGAGACC | | | |
| | | ACGTCTTACAGACA | | | |
| | | CTTAA | | | |
| Tyrosine- | PEST | ATGGAGCAAGTGG | 260 | MEQVEI | 261 |
| protein | (E57D)₃₀₆ | AGATCCTGAGGAA | | LRKFIQ | |
| phospha- | | ATTCATCCAGAGGG | | RVQAMK | |
| tase | | TCCAGGCCATGAAG | | SPDHNG | |
| non- | | AGTCCTGACCACAA | | EDNFAR | |
| receptor | | TGGGGAGGACAAC | | DFMRLR | |
| type 12 | | TTCGCCCGGGACTT | | RLSTKY | |
| | | CATGCGGTTAAGAA | | RTEKIY | |
| | | GATTGTCTACCAAA | | PTATGE | |
| | | TATAGAACAGAAA | | KEDNVK | |
| | | AGATATATCCCACA | | KNRYKD | |
| | | GCCACTGGAGAAA | | ILPFDH | |
| | | AAGAAGACAATGT | | SRVKLT | |
| | | TAAAAAGAACAGA | | LKTPSQ | |
| | | TACAAGGACATACT | | DSDYIN | |
| | | GCCATTTGATCACA | | ANFIKG | |
| | | GCCGAGTTAAATTG | | VYGPKA | |
| | | ACATTAAAGACTCC | | YVATQG | |
| | | TTCACAAGATTCAG | | PLANTV | |
| | | ACTATATCAATGCA | | IDFWRM | |
| | | AATTTTATAAAGGG | | IWEYNV | |
| | | CGTCTATGGGCCAA | | VIIVMA | |
| | | AAGCATATGTAGCA | | CREFEM | |
| | | ACTCAAGGACCTTT | | GRKKCE | |
| | | AGCAAATACAGTA | | RYWPLY | |
| | | ATAGATTTTTGGAG | | GEDPIT | |
| | | GATGATATGGGAGT | | FAPFKI | |
| | | ATAATGTTGTGATC | | SCEDEQ | |
| | | ATTGTAATGGCCTG | | ARTDYF | |
| | | CCGAGAATTTGAGA | | IRTLLL | |
| | | TGGGAAGGAAAAA | | EFQNES | |
| | | ATGTGAGCGCTATT | | RRLYQF | |
| | | GGCCTTTGTATGGA | | HYVNWP | |
| | | GAAGACCCCATAA | | DHDVPS | |
| | | CGTTTGCACCATTT | | SFDSIL | |
| | | AAAATTTCTTGTGA | | DMISLM | |
| | | GGATGAACAAGCA | | RKYQEH | |
| | | AGAACAGACTACTT | | EDVPIC | |
| | | CATCAGGACACTCT | | IHCSAG | |
| | | TACTTGAATTTCAA | | CGRTGA | |
| | | AATGAATCTCGTAG | | ICAIDY | |
| | | GCTGTATCAGTTTC | | TWNLLK | |
| | | ATTATGTGAACTGG | | AGKIPE | |
| | | CCAGACCATGATGT | | EFNVFN | |
| | | TCCTTCATCATTTG | | LIQEMR | |
| | | ATTCTATTCTGGAC | | TQRHSA | |
| | | ATGATAAGCTTAAT | | VQTKEQ | |
| | | GAGGAAATATCAA | | YELVHR | |
| | | GAACATGAAGATGT | | AIAQLF | |
| | | TCCTATTTGTATTC | | EKQLQL | |
| | | ATTGCAGTGCAGGC | | YEIHGA | |
| | | TGTGGAAGAACAG | | * | |
| | | GTGCCATTTGTGCC | | | |
| | | ATAGATTATACGTG | | | |
| | | GAATTTACTAAAAG | | | |
| | | CTGGGAAAATACC | | | |
| | | AGAGGAATTTAATG | | | |
| | | TATTTAATTTAATA | | | |
| | | CAAGAAATGAGAA | | | |
| | | CACAAAGGCATTCT | | | |
| | | GCAGTACAAACAA | | | |
| | | AGGAGCAATATGA | | | |
| | | ACTTGTTCATAGAG | | | |
| | | CTATTGCCCAACTG | | | |
| | | TTTGAAAAACAGCT | | | |
| | | ACAACTATATGAAA | | | |
| | | TTCATGGAGCTTAA | | | |
| Striatum- | STEP₂₈₂₋₅₆₃ | ATGTCTTCTGGTGT | 262 | MSSGVD | 263 |
| enriched | | AGATCTGGGTACCG | | LGTENL | |
| protein- | | AGAACCTGTACTTC | | YFQSMS | |
| tyrosine | | CAATCCATGTCCCG | | RVLQAE | |
| phospha- | | TGTCCTCCAAGCAG | | ELHEKA | |
| tase | | AAGAGCTTCATGAA | | LDPFLL | |
| catalytic | | AAGGCCCTGGACCC | | QAEFFE | |
| domain | | TTTCCTGCTGCAGG | | IPMNFV | |
| | | CGGAATTCTTTGAA | | DPKEYD | |
| | | ATCCCCATGAACTT | | IPGLVR | |
| | | TGTGGATCCGAAAG | | KNRYKT | |
| | | AGTACGACATCCCT | | ILPNPH | |
| | | GGGCTGGTGCGGA | | SRVCLT | |
| | | AGAACCGGTACAA | | SPDPDD | |
| | | AACCATACTTCCCA | | PLSSYI | |
| | | ACCCTCACAGCAGA | | NANYIR | |
| | | GTGTGTCTGACCTC | | GYGGEE | |
| | | ACCAGACCCTGACG | | KVYIAT | |
| | | ACCCTCTGAGTTCC | | QGPIVS | |
| | | TACATCAATGCCAA | | TVADFW | |
| | | CTACATCCGGGGCT | | RMVWQE | |
| | | ATGGTGGGGAGGA | | HTPIIV | |
| | | GAAGGTGTACATCG | | MITNIE | |
| | | CCACTCAGGGACCC | | EMNEKC | |
| | | ATCGTCAGCACGGT | | TEYWPE | |
| | | CGCCGACTTCTGGC | | EQVAYD | |
| | | GCATGGTGTGGCAG | | GVEITV | |
| | | GAGCACACGCCCAT | | QKVIHT | |
| | | CATTGTCATGATCA | | EDYRLR | |
| | | CCAACATCGAGGA | | LISLKS | |
| | | GATGAACGAGAAA | | GTEERG | |
| | | TGCACCGAGTATTG | | LKHYWF | |
| | | GCCGGAGGAGCAG | | TSWPDQ | |
| | | GTGGCGTACGACG | | KTPDRA | |
| | | GTGTTGAGATCACT | | PPLLHL | |
| | | GTGCAGAAAGTCAT | | VREVEE | |
| | | TCACACGGAGGATT | | AAQQEG | |
| | | ACCGGCTGCGACTC | | PHCAPI | |
| | | ATCTCCCTCAAGAG | | IVHCSA | |
| | | TGGGACTGAGGAG | | GIGRTG | |
| | | CGAGGCCTGAAGC | | CFIATS | |
| | | ATTACTGGTTCACA | | ICCQQL | |
| | | TCCTGGCCCGACCA | | RQEGVV | |
| | | GAAGACCCCAGAC | | DILKTT | |
| | | CGGGCCCCCCCACT | | CQLRQD | |
| | | CCTGCACCTGGTGC | | RGGMIQ | |
| | | GGGAGGTGGAGGA | | TCEQYQ | |
| | | GGCAGCCCAGCAG | | FVHHVM | |
| | | GAGGGGCCCCACT | | SLYEKQ | |
| | | GTGCCCCCATCATC | | LSHQS* | |
| | | GTCCACTGCAGTGC | | | |
| | | AGGGATTGGGAGG | | | |
| | | ACCGGCTGCTTCAT | | | |
| | | TGCCACCAGCATCT | | | |
| | | GCTGCCAGCAGCTG | | | |
| | | CGGCAGGAGGGTG | | | |
| | | TAGTGGACATCCTG | | | |
| | | AAGACCACGTGCC | | | |
| | | AGCTCCGTCAGGAC | | | |
| | | AGGGGCGGCATGA | | | |
| | | TCCAGACATGCGAG | | | |
| | | CAGTACCAGTTTGT | | | |
| | | GCACCACGTCATGA | | | |
| | | GCCTCTACGAAAAG | | | |
| | | CAGCTGTCCCACCA | | | |
| | | GTCCTGA | | | |
| Tyrosine- | SHP2₂₃₇₋₅₂₉ | ATGGCTGAGACCAC | 264 | MAETTD | 265 |
| protein | | AGATAAAGTCAAA | | KVKQGF | |
| phospha- | | CAAGGCTTTTGGGA | | WEEFET | |
| tase | | AGAATTTGAGACAC | | LQQQEC | |
| non- | | TACAACAACAGGA | | KLLYSR | |
| receptor | | GTGCAAACTTCTCT | | KEGQRQ | |
| type 11 | | ACAGCCGAAAAGA | | ENKNKN | |
| | | GGGTCAAAGGCAA | | RYKNIL | |
| | | GAAAACAAAAACA | | PFDHTR | |
| | | AAAATAGATATAA | | VVLHDG | |
| | | AAACATCCTGCCCT | | DPNEPV | |
| | | TTGATCATACCAGG | | SDYINA | |
| | | GTTGTCCTACACGA | | NIIMPE | |
| | | TGGTGATCCCAATG | | FETKCN | |
| | | AGCCTGTTTCAGAT | | NSKPKK | |
| | | TACATCAATGCAAA | | SYIATQ | |
| | | TATCATCATGCCTG | | GCLQNT | |
| | | AATTTGAAACCAAG | | VNDFWR | |
| | | TGCAACAATTCAAA | | MVFQEN | |
| | | GCCCAAAAAGAGT | | SRVIVM | |
| | | TACATTGCCACACA | | TTKEVE | |
| | | AGGCTGCCTGCAAA | | RGKSKC | |
| | | ACACGGTGAATGA | | VKYWPD | |
| | | CTTTTGGCGGATGG | | EYALKE | |
| | | TGTTCCAAGAAAAC | | YGVMRV | |
| | | TCCCGAGTGATTGT | | RNVKES | |
| | | CATGACAACGAAA | | AAHDYT | |
| | | GAAGTGGAGAGAG | | LRELKL | |
| | | GAAAGAGTAAATG | | SKVGQG | |
| | | TGTCAAATACTGGC | | NTERTV | |
| | | CTGATGAGTATGCT | | WQYHFR | |
| | | CTAAAAGAATATG | | TWPDHG | |
| | | GCGTCATGCGTGTT | | VPSDPG | |
| | | AGGAACGTCAAAG | | GVLDFL | |
| | | AAAGCGCCGCTCAT | | EEVHHK | |
| | | GACTATACGCTAAG | | QESIMD | |
| | | AGAACTTAAACTTT | | AGPVVV | |
| | | CAAAGGTTGGACA | | HCSAGI | |
| | | AGGGAATACGGAG | | GRTGTF | |
| | | AGAACGGTCTGGC | | IVIDIL | |
| | | AATACCACTTTCGG | | IDIIRE | |
| | | ACCTGGCCGGACCA | | KGVDCD | |
| | | CGGCGTGCCCAGCG | | IDVPKT | |
| | | ACCCTGGGGGCGTG | | IQMVRS | |
| | | CTGGACTTCCTGGA | | QRSGMV | |
| | | GGAGGTGCACCAT | | QTEAQY | |
| | | AAGCAGGAGAGCA | | RFIYMA | |
| | | TCATGGATGCAGGG | | VQHYIE | |
| | | CCGGTCGTGGTGCA | | TLQRRI | |
| | | CTGCAGTGCTGGAA | | * | |
| | | TTGGCCGGACAGG | | | |
| | | GACGTTCATTGTGA | | | |
| | | TTGATATTCTTATT | | | |
| | | GACATCATCAGAG | | | |
| | | AGAAAGGTGTTGA | | | |
| | | CTGCGATATTGACG | | | |
| | | TTCCCAAAACCATC | | | |
| | | CAGATGGTGCGGTC | | | |
| | | TCAGAGGTCAGGG | | | |
| | | ATGGTCCAGACAG | | | |
| | | AAGCACAGTACCG | | | |
| | | ATTTATCTATATGG | | | |
| | | CGGTCCAGCATTAT | | | |
| | | ATTGAAACACTACA | | | |
| | | GCGCAGGATTTGA | | | |
| Choline | ScCk | ATGGTGCAGGAGTC | 266 | MVQESR | 267 |
| kinase | | CCGCCCCGGCTCGG | | PGSVRS | |
| from S. | | TCCGGTCGTATTCC | | YSVGYQ | |
| cerevisiae | | GTGGGCTACCAGGC | | ARSRSS | |
| | | CCGGTCGCGGTCGT | | SQRRHS | |
| | | CGTCCCAGCGCCGC | | LTRQRS | |
| | | CATTCGCTCACGCG | | SQRLIR | |
| | | GCAGCGCAGCAGC | | TISIES | |
| | | CAGCGGCTCATCCG | | DVSNIT | |
| | | GACGATCTCCATCG | | DDDDLR | |
| | | AGAGCGATGTGAG | | AVNEGV | |
| | | CAATATCACGGACG | | AGVQLD | |
| | | ATGATGATCTGCGG | | VSETAN | |
| | | GCGGTGAATGAAG | | KGPRRA | |
| | | GGGTGGCCGGGGT | | SATDVT | |
| | | CCAGCTCGACGTCT | | DSLGST | |
| | | CCGAGACGGCGAA | | SSEYIE | |
| | | CAAAGGGCCACGC | | IPFVKE | |
| | | CGGGCCAGTGCCAC | | TLDASL | |
| | | CGATGTCACCGACT | | PSDYLK | |
| | | CGCTGGGCTCCACG | | QDILNL | |
| | | TCCAGCGAATATAT | | IQSLKI | |
| | | CGAGATCCCCTTCG | | SKWYNN | |
| | | TGAAAGAGACGCT | | KKIQPV | |
| | | GGACGCGAGCCTCC | | AQDMNL | |
| | | CCTCGGATTACCTC | | VKISGA | |
| | | AAACAAGACATCCT | | MTNAIF | |
| | | GAACCTGATCCAAT | | KVEYPK | |
| | | CCCTGAAGATCTCG | | LPSLLL | |
| | | AAATGGTACAATA | | RIYGPN | |
| | | ACAAAAAGATCCA | | IDNIID | |
| | | GCCCGTCGCCCAGG | | REYELQ | |
| | | ACATGAACCTCGTC | | ILARLS | |
| | | AAAATCTCCGGCGC | | LKNIGP | |
| | | GATGACCAATGCG | | SLYGCF | |
| | | ATCTTCAAGGTGGA | | VNGRFE | |
| | | GTACCCGAAACTGC | | QFLENS | |
| | | CGTCCCTCCTGCTG | | KTLTKD | |
| | | CGGATCTATGGCCC | | DIRNWK | |
| | | GAATATCGATAACA | | NSQRIA | |
| | | TCATCGACCGCGAA | | RRMKEL | |
| | | TATGAACTCCAGAT | | HVGVPL | |
| | | CCTCGCGCGGCTCT | | LSSERK | |
| | | CGCTGAAAAACATC | | NGSACW | |
| | | GGGCCGTCCCTGTA | | QKINQW | |
| | | CGGCTGCTTCGTGA | | LRTIEK | |
| | | ATGGGCGCTTCGAG | | VDQWVG | |
| | | CAGTTCCTCGAAAA | | DPKNIE | |
| | | CTCCAAAACGCTGA | | NSLLCE | |
| | | CCAAGGATGATATC | | NWSKFM | |
| | | CGGAACTGGAAAA | | DIVDRY | |
| | | ACTCGCAACGGATC | | HKWLIS | |
| | | GCCCGCCGCATGAA | | QEQGIE | |
| | | GGAGCTGCATGTGG | | QVNKNL | |
| | | GCGTGCCCCTCCTC | | IFCHND | |
| | | TCGTCGGAGCGGA | | AQYGNL | |
| | | AGAATGGGAGCGC | | LFTAPV | |
| | | CTGCTGGCAAAAA | | MNTPSL | |
| | | ATCAACCAATGGCT | | YTAPSS | |
| | | CCGCACGATCGAG | | TSLTSQ | |
| | | AAGGTGGATCAGT | | SSSLFP | |
| | | GGGTCGGGGACCC | | SSSNVI | |
| | | GAAGAACATCGAG | | VDDIIN | |
| | | AACAGCCTCCTCTG | | PPKQEQ | |
| | | CGAAAATTGGTCCA | | SQDSKL | |
| | | AATTCATGGACATC | | VVIDFE | |
| | | GTCGATCGGTACCA | | YAGANP | |
| | | CAAGTGGCTGATCA | | AAYDLA | |
| | | GCCAAGAACAAGG | | NHLSEW | |
| | | GATCGAGCAAGTC | | MYDYNN | |
| | | AACAAAAATCTGAT | | AKAPHQ | |
| | | CTTCTGCCATAATG | | CHADRY | |
| | | ATGCCCAATACGGG | | PDKEQV | |
| | | AATCTCCTCTTCAC | | LNFLYS | |
| | | CGCGCCCGTCATGA | | YVSHLR | |
| | | ACACCCCCTCCCTG | | GGAKEP | |
| | | TATACCGCGCCGAG | | IDEEVQ | |
| | | CTCGACCTCCCTGA | | RLYKSI | |
| | | CGTCCCAAAGCAGC | | IQWRPT | |
| | | AGCCTCTTCCCCTC | | VQLFWS | |
| | | GTCCAGCAACGTGA | | LWAILQ | |
| | | TCGTCGATGATATC | | SGKLEK | |
| | | ATCAATCCCCCGAA | | KEASTA | |
| | | GCAAGAACAATCC | | ITREEI | |
| | | CAAGATTCCAAACT | | GPNGKK | |
| | | CGTGGTCATCGATT | | YIIKTE | |
| | | TCGAATACGCCGGG | | PESPEE | |
| | | GCCAATCCCGCCGC | | DFVEND | |
| | | GTACGATCTCGCCA | | DEPEAG | |
| | | ATCACCTCTCGGAA | | VSIDTF | |
| | | TGGATGTACGACTA | | DYMAYG | |
| | | TAATAACGCCAAA | | RDKIAV | |
| | | GCCCCGCACCAGTG | | FWGDLI | |
| | | CCACGCCGACCGGT | | GLGIIT | |
| | | ACCCCGACAAGGA | | EEECKN | |
| | | GCAAGTGCTCAACT | | FSSFKF | |
| | | TCCTGTATTCGTAT | | LDTSYL | |
| | | GTCAGCCATCTCCG | | * | |
| | | CGGCGGGGCCAAA | | | |
| | | GAGCCCATCGATGA | | | |
| | | AGAAGTCCAGCGC | | | |
| | | CTCTATAAATCGAT | | | |
| | | CATCCAGTGGCGCC | | | |
| | | CCACGGTGCAGCTC | | | |
| | | TTCTGGTCGCTGTG | | | |
| | | GGCGATCCTGCAAA | | | |
| | | GCGGCAAGCTGGA | | | |
| | | AAAAAAAGAAGCC | | | |
| | | AGCACCGCCATCAC | | | |
| | | CCGCGAAGAAATC | | | |
| | | GGGCCCAATGGGA | | | |
| | | AAAAGTATATCATC | | | |
| | | AAGACGGAGCCCG | | | |
| | | AGTCGCCCGAAGA | | | |
| | | GGACTTCGTCGAAA | | | |
| | | ATGACGACGAACC | | | |
| | | CGAAGCCGGCGTGT | | | |
| | | CGATCGATACCTTC | | | |
| | | GACTACATGGCCTA | | | |
| | | CGGGCGGGACAAG | | | |
| | | ATCGCGGTGTTCTG | | | |
| | | GGGGGACCTGATC | | | |
| | | GGGCTGGGCATCAT | | | |
| | | CACGGAGGAGGAA | | | |
| | | TGCAAGAACTTCTC | | | |
| | | GAGCTTCAAATTCC | | | |
| | | TCGACACCAGCTAC | | | |
| | | CTGTAA | | | |
| Isopen- | AtIPK | ATGGAACTCAATAT | 268 | MELNIS | 269 |
| tenyl | | CAGCGAAAGCCGG | | ESRSRS | |
| kinase | | TCGCGCAGCATCCG | | IRCIVK | |
| from | | GTGCATCGTGAAGC | | LGGAAI | |
| A. | | TCGGGGGCGCGGC | | TCKNEL | |
| thaliana | | CATCACCTGCAAAA | | EKIHDE | |
| | | ACGAACTCGAAAA | | NLEVVA | |
| | | GATCCATGACGAA | | CQLRQA | |
| | | AACCTCGAAGTGGT | | MLEGSA | |
| | | GGCCTGCCAACTGC | | PSKVIG | |
| | | GGCAAGCGATGCT | | MDWSKR | |
| | | GGAAGGCTCCGCCC | | PGSSEI | |
| | | CCTCCAAAGTCATC | | SCDVDD | |
| | | GGCATGGACTGGTC | | IGDQKS | |
| | | CAAACGGCCGGGC | | SEFSKF | |
| | | TCCTCCGAAATCTC | | VVVHGA | |
| | | CTGCGATGTGGACG | | GSFGHF | |
| | | ACATCGGCGACCA | | QASRSG | |
| | | GAAATCGAGCGAA | | VHKGGL | |
| | | TTCTCGAAGTTCGT | | EKPIVK | |
| | | GGTCGTCCACGGGG | | AGFVAT | |
| | | CGGGCTCGTTCGGC | | RISVTN | |
| | | CATTTCCAAGCGTC | | LNLEIV | |
| | | GCGGTCGGGCGTCC | | RALARE | |
| | | ATAAAGGGGGCCT | | GIPTIG | |
| | | GGAGAAGCCCATC | | MSPFSC | |
| | | GTCAAAGCCGGGTT | | GWSTSK | |
| | | CGTCGCGACCCGGA | | RDVASA | |
| | | TCTCGGTCACCAAT | | DLATVA | |
| | | CTCAATCTCGAGAT | | KTIDSG | |
| | | CGTCCGCGCCCTGG | | FVPVLH | |
| | | CCCGGGAAGGCAT | | GDAVLD | |
| | | CCCGACGATCGGG | | NILGCT | |
| | | ATGAGCCCCTTCAG | | ILSGDV | |
| | | CTGCGGCTGGTCCA | | IIRHLA | |
| | | CCTCCAAACGGGAT | | DHLKPE | |
| | | GTCGCGTCGGCGGA | | YVVFLT | |
| | | TCTCGCGACGGTCG | | DVLGVY | |
| | | CCAAGACCATCGAC | | DRPPSP | |
| | | TCGGGCTTCGTGCC | | SEPDAV | |
| | | CGTGCTCCATGGGG | | LLKEIA | |
| | | ACGCGGTCCTGGAC | | VGEDGS | |
| | | AACATCCTGGGCTG | | WKVVNP | |
| | | CACGATCCTGTCCG | | LLEHTD | |
| | | GCGATGTCATCATC | | KKVDYS | |
| | | CGGCACCTGGCCGA | | VAAHDT | |
| | | CCATCTCAAGCCGG | | TGGMET | |
| | | AATACGTCGTGTTC | | KISEAA | |
| | | CTGACCGACGTGCT | | MIAKLG | |
| | | CGGGGTCTATGACC | | VDVYIV | |
| | | GGCCCCCGAGCCCG | | KAATTH | |
| | | TCGGAGCCGGACG | | SQRALN | |
| | | CCGTCCTGCTCAAG | | GDLRDS | |
| | | GAGATCGCCGTCGG | | VPEDWL | |
| | | GGAGGATGGCTCCT | | GTIIRF | |
| | | GGAAGGTGGTCAA | | SK* | |
| | | CCCCCTCCTGGAAC | | | |
| | | ATACCGACAAAAA | | | |
| | | GGTGGATTATTCCG | | | |
| | | TCGCGGCCCACGAT | | | |
| | | ACCACGGGGGGGA | | | |
| | | TGGAAACCAAAAT | | | |
| | | CTCGGAAGCCGCCA | | | |
| | | TGATCGCCAAGCTC | | | |
| | | GGCGTGGATGTGTA | | | |
| | | CATCGTGAAAGCCG | | | |
| | | CGACCACCCATTCG | | | |
| | | CAGCGGGCGCTCA | | | |
| | | ATGGCGACCTGCGC | | | |
| | | GACTCCGTCCCCGA | | | |
| | | AGACTGGCTGGGG | | | |
| | | ACCATCATCCGCTT | | | |
| | | CAGCAAATAA | | | |
| Isopentenyl | idi | ATGCAAACGGAAC | 270 | MQTEHV | 271 |
| diphosphate | | ACGTCATTTTATTG | | ILLNAQ | |
| Delta- | | AATGCACAGGGAG | | GVPTGT | |
| isomerase | | TTCCCACGGGTACG | | LEKYAA | |
| from | | CTGGAAAAGTATGC | | HTADTR | |
| E. coli | | CGCACACACGGCA | | LHLAFS | |
| | | GACACCCGCTTACA | | SWLFNA | |
| | | TCTCGCGTTCTCCA | | KGQLLV | |
| | | GTTGGCTGTTTAAT | | TRRALS | |
| | | GCCAAAGGACAAT | | KKAWPG | |
| | | TATTAGTTACCCGC | | VWTNSV | |
| | | CGCGCACTGAGCA | | CGHPQL | |
| | | AAAAAGCATGGCC | | GESNED | |
| | | TGGCGTGTGGACTA | | AVIRRC | |
| | | ACTCGGTTTGTGGG | | RYELGV | |
| | | CACCCACAACTGGG | | EITPPE | |
| | | AGAAAGCAACGAA | | SIYPDF | |
| | | GACGCAGTGATCCG | | RYRATD | |
| | | CCGTTGCCGTTATG | | PSGIVE | |
| | | AGCTTGGCGTGGAA | | NEVCPV | |
| | | ATTACGCCTCCTGA | | FAARTT | |
| | | ATCTATCTATCCTG | | SALQIN | |
| | | ACTTTCGCTACCGC | | DDEVMD | |
| | | GCCACCGATCCGAG | | YQWCDL | |
| | | TGGCATTGTGGAAA | | ADVLHG | |
| | | ATGAAGTGTGTCCG | | IDATPW | |
| | | GTATTTGCCGCACG | | AFSPWM | |
| | | CACCACTAGTGCGT | | VMQATN | |
| | | TACAGATCAATGAT | | REARKR | |
| | | GATGAAGTGATGG | | LSAFTQ | |
| | | ATTATCAATGGTGT | | LK* | |
| | | GATTTAGCAGATGT | | | |
| | | ATTACACGGTATTG | | | |
| | | ATGCCACGCCGTGG | | | |
| | | GCGTTCAGTCCGTG | | | |
| | | GATGGTGATGCAG | | | |
| | | GCGACAAATCGCG | | | |
| | | AAGCCAGAAAACG | | | |
| | | ATTATCTGCATTTA | | | |
| | | CCCAGCTTAAATAA | | | |
| Superbinder | sFynSH | TGGTACTTTGGCAA | 272 | WYFGKL | 273 |
| Fyn SH2 | 2 | ACTTGGGCGTAAAG | | GRKDAE | |
| | | ATGCGGAGCGTCA | | RQLLSF | |
| | | ACTTCTGTCCTTTG | | GNPRGT | |
| | | GAAATCCCCGTGGA | | FLIRES | |
| | | ACCTTCTTGATCCG | | ETVKGA | |
| | | TGAGTCTGAAACGG | | YALSIR | |
| | | TCAAGGGCGCATAT | | DWDDMK | |
| | | GCTCTGAGTATCCG | | GDHVKH | |
| | | CGACTGGGACGAT | | YLIRKL | |
| | | ATGAAGGGAGATC | | DNGGYY | |
| | | ACGTAAAACATTAT | | ITTRAQ | |
| | | CTTATTCGCAAGTT | | FETLQQ | |
| | | GGATAACGGGGGA | | LVQHYS | |
| | | TACTACATTACAAC | | ERAAGL | |
| | | GCGCGCGCAGTTTG | | SSRLVP | |
| | | AGACCTTGCAGCAA | | PS | |
| | | CTGGTTCAGCACTA | | | |
| | | TAGTGAGCGTGCCG | | | |
| | | CGGGTCTGAGTAGC | | | |
| | | CGCTTGGTGCCTCC | | | |
| | | TTCC | | | |
| Superbinder | sBlkSH | TGGTTCTTCCGCAG | 274 | WFFRSQ | 275 |
| Blk SH2 | 2 | TCAGGGGCGTAAG | | GRKEAE | |
| | | GAGGCTGAGCGTC | | RQLLAP | |
| | | AGTTATTAGCCCCG | | INKAGS | |
| | | ATTAATAAGGCCGG | | FLIRES | |
| | | GTCCTTTTTGATCC | | ETVKGA | |
| | | GCGAGTCAGAGAC | | FALSVK | |
| | | TGTCAAAGGCGCGT | | DVTTQG | |
| | | TTGCCTTAAGTGTC | | ELIKHY | |
| | | AAAGACGTCACTAC | | LIRCLD | |
| | | GCAAGGCGAGCTT | | EGGYYI | |
| | | ATTAAACACTATCT | | SPRITF | |
| | | TATTCGTTGCCTGG | | PSLQAL | |
| | | ACGAAGGAGGGTA | | VQHYSK | |
| | | CTACATTAGCCCAC | | KGDGLC | |
| | | GCATTACCTTCCCC | | QRLTLP | |
| | | AGTTTACAAGCATT | | C | |
| | | AGTTCAACATTACT | | | |
| | | CAAAGAAAGGAGA | | | |
| | | CGGTTTATGTCAGC | | | |
| | | GCTTAACGTTACCG | | | |
| | | TGC | | | |
| Superbinder | c | TGGTATATGGGTCC | 276 | WYMGPV | 277 |
| Crkl SH2 | | CGTGAGCCGTCAGG | | SRQEAQ | |
| | | AAGCACAGACCCG | | TRLQGQ | |
| | | CTTGCAGGGTCAGC | | RHGMFL | |
| | | GTCACGGCATGTTT | | VRDSET | |
| | | TTAGTGCGCGACTC | | VKGDYA | |
| | | TGAAACGGTCAAA | | LSVSEN | |
| | | GGTGACTACGCTTT | | SRVSHY | |
| | | GTCAGTTAGTGAAA | | LINSLP | |
| | | ATAGCCGTGTGTCG | | NRRFKI | |
| | | CATTATTTAATTAA | | GDQEFD | |
| | | CTCTTTACCAAATC | | HLPALL | |
| | | GTCGCTTTAAAATC | | EFYKIH | |
| | | GGAGATCAAGAGT | | YLDTTT | |
| | | TCGATCACTTACCT | | LIEPA | |
| | | GCTTTACTTGAATT | | | |
| | | TTATAAAATCCATT | | | |
| | | ATCTTGATACCACG | | | |
| | | ACACTGATCGAACC | | | |
| | | AGCG | | | |
| Superbinder | sPTN6_ | TGGTATCACGGACA | 278 | WYHGHM | 279 |
| PTPN6_C_ | CSH2 | CATGTCCCGTGGTC | | SRGQAE | |
| SH2 | | AGGCGGAAACCCTT | | TLLQAK | |
| | | CTGCAGGCGAAAG | | GEPWTF | |
| | | GCGAGCCCTGGACC | | LVRESE | |
| | | TTCTTAGTACGCGA | | TVKGDF | |
| | | GAGCGAAACAGTT | | ALSVLS | |
| | | AAAGGGGATTTCGC | | DQPKAG | |
| | | ATTATCGGTACTTA | | PGSPLR | |
| | | GTGACCAGCCTAAG | | VTHILV | |
| | | GCCGGGCCTGGAA | | MCEGGR | |
| | | GCCCTTTACGCGTA | | YTVGGL | |
| | | ACTCACATTTTAGT | | ETFDSL | |
| | | AATGTGCGAGGGT | | TDLVEH | |
| | | GGACGTTATACCGT | | FKKTGI | |
| | | CGGCGGATTGGAG | | EEASGA | |
| | | ACGTTCGATAGCCT | | FVYLRQ | |
| | | TACCGACTTGGTTG | | PY | |
| | | AGCATTTCAAAAAG | | | |
| | | ACTGGCATCGAAG | | | |
| | | AAGCGTCAGGAGC | | | |
| | | TTTCGTTTACTTGC | | | |
| | | GTCAGCCGTAT | | | |
| LAT | LATY1 | TGCGAAGACGCCG | 280 | CEDADE | 281 |
| substrate | 27 | ATGAGGACGAGGA | | DEDDYH | |
| Y127 | | TGACTATCACAACC | | NPGYLV | |
| | | CTGGATACCTTGTT | | VLP | |
| | | GTGCTGCCT | | | |
| SLP76 | SLP76 | GGAGGATGGTCCTC | 282 | GGWSSF | 283 |
| substrate | Y113 | CTTCGAGGAGGAC | | EEDDYE | |
| Y113 | | GACTACGAGTCTCC | | SPNDDQ | |
| | | CAACGACGACCAA | | DGE | |
| | | GACGGGGAG | | | |
| CD6 | CD6Y6 | CCACAACCAGACA | 284 | PQPDST | 285 |
| substrate | 62 | GTACAGACAACGA | | DNDDYD | |
| Y662 | | CGATTACGATGACA | | DISAA | |
| | | TTTCTGCAGCG | | | |
| Ptn6_C | Ptn6_C | CAAGGCGTGATTTA | 286 | QGVIYS | 287 |
| substrate | | CTCAGACTTAAACC | | DLNLP | |
| | | TGCCT | | | |

TABLE 29
Ubiquitin components

| | | | SEQ |
|---|---|---|---|
| Component | Name | Sequence | ID |
| Ubiquitin | USP11 | MAVAPRLFGGLCFRFRDQNPEVAV | 288 |
| carboxyl- | | EGRLPISHSCVGCRRERTAMATVA | |
| terminal | | ANPAAAAAAVAAAAAVTEDREPQH | |
| hydrolase | | EELPGLDSQWRQIENGESGRERPL | |
| 11 | | RAGESWFLVEKHWYKQWEAYVQGG | |
| | | DQDSSTFPGCINNATLFQDEINWR | |
| | | LKEGLVEGEDYVLLPAAAWHYLVS | |
| | | WYGLEHGQPPIERKVIELPNIQKV | |
| | | EVYPVELLLVRHNDLGKSHTVQFS | |
| | | HTDSIGLVLRTARERFLVEPQEDT | |
| | | RLWAKNSEGSLDRLYDTHITVLDA | |
| | | ALETGQLIIMETRKKDGTWPSAQL | |
| | | HVMNNNMSEEDEDFKGQPGICGLT | |
| | | NLGNTCFMNSALQCLSNVPQLTEY | |
| | | FLNNCYLEELNFRNPLGMKGEIAE | |
| | | AYADLVKQAWSGHHRSIVPHVFKN | |
| | | KVGHFASQFLGYQQHDSQELLSFL | |
| | | LDGLHEDLNRVKKKEYVELCDAAG | |
| | | RPDQEVAQEAWQNHKRRNDSVIVD | |
| | | TFHGLFKSTLVCPDCGNVSVTFDP | |
| | | FCYLSVPLPISHKRVLEVFFIPMD | |
| | | PRRKPEQHRLVVPKKGKISDLCVA | |
| | | LSKHTGISPERMMVADVFSHRFYK | |
| | | LYQLEEPLSSILDRDDIFVYEVSG | |
| | | RIEAIEGSREDIVVPVYLRERTPA | |
| | | RDYNNSYYGLMLFGHPLLVSVPRD | |
| | | RFTWEGLYNVLMYRLSRYVTKPNS | |
| | | DDEDDGDEKEDDEEDKDDVPGPST | |
| | | GGSLRDPEPEQAGPSSGVTNRCPF | |
| | | LLDNCLGTSQWPPRRRRKQLFTLQ | |
| | | TVNSNGTSDRTTSPEEVHAQPYIA | |
| | | IDWEPEMKKRYYDEVEAEGYVKHD | |
| | | CVGYVMKKAPVRLQECIELFTTVE | |
| | | TLEKENPWYCPSCKQHQLATKKLD | |
| | | LWMLPEILIIHLKRFSYTKFSREK | |
| | | LDTLVEFPIRDLDFSEFVIQPQNE | |
| | | SNPELYKYDLIAVSNHYGGMRDGH | |
| | | YTTFACNKDSGQWHYFDDNSVSPV | |
| | | NENQIESKAAYVLFYQRQDVARRL | |
| | | LSPAGSSGAPASPACSSPPSSEFM | |
| | | DVN* | |
| Ubiquitin | USP14 | MPLYSVTVKWGKEKFEGVELNTDE | 289 |
| carboxyl- | | PPMVFKAQLFALTGVQPARQKVMV | |
| terminal | | KGGTLKDDDWGNIKIKNGMTLLMM | |
| hydrolase | | GSADALPEEPSAKTVFVEDMTEEQ | |
| 14 | | LASAMELPCGLTNLGNTCYMNATV | |
| | | QCIRSVPELKDALKRYAGALRASG | |
| | | EMASAQYITAALRDLFDSMDKTSS | |
| | | SIPPIILLQFLHMAFPQFAEKGEQ | |
| | | GQYLQQDANECWIQMMRVLQQKLE | |
| | | AIEDDSVKETDSSSASAATPSKKK | |
| | | SLIDQFFGVEFETTMKCTESEEEE | |
| | | VTKGKENQLQLSCFINQEVKYLFT | |
| | | GLKLRLQEEITKQSPTLQRNALYI | |
| | | KSSKISRLPAYLTIQMVRFFYKEK | |
| | | ESVNAKVLKDVKFPLMLDMYELCT | |
| | | PELQEKMVSFRSKFKDLEDKKVNQ | |
| | | QPNTSDKKSSPQKEVKYEPFSFAD | |
| | | DIGSNNCGYYDLQAVLTHQGRSSS | |
| | | SGHYVSWVKRKQDEWIKFDDDKVS | |
| | | IVTPEDILRLSGGGDWHIAYVLLY | |
| | | GPRRVEIMEEESEQ* | |
| Ovarian | OTUD7B | MTLDMDAVLSDFVRSTGAEPGLAR | 290 |
| tumor | | DLLEGKNWDVNAALSDFEQLRQVH | |
| (OTU) | | AGNLPPSFSEGSGGSRTPEKGFSD | |
| domain- | | REPTRPPRPILQRQDDIVQEKRLS | |
| containing | | RGISHASSSIVSLARSHVSSNGGG | |
| protein 7B | | GGSNEHPLEMPICAFQLPDLTVYN | |
| | | EDFRSFIERDLIEQSMLVALEQAG | |
| | | RLNWWVSVDPTSQRLLPLATTGDG | |
| | | NCLLHAASLGMWGFHDRDLMLRKA | |
| | | LYALMEKGVEKEALKRRWRWQQTQ | |
| | | QNKESGLVYTEDEWQKEWNELIKL | |
| | | ASSEPRMHLGTNGANCGGVESSEE | |
| | | PVYESLEEFHVFVLAHVLRRPIVV | |
| | | VADTMLRDSGGEAFAPIPFGGIYL | |
| | | PLEVPASQCHRSPLVLAYDQAHFS | |
| | | ALVSMEQKENTKEQAVIPLTDSEY | |
| | | KLLPLHFAVDPGKGWEWGKDDSDN | |
| | | VRLASVILSLEVKLHLLHSYMNVK | |
| | | WIPLSSDAQAPLAQPESPTASAGD | |
| | | EPRSTPESGDSDKESVGSSSTSNE | |
| | | GGRRKEKSKRDREKDKKRADSVAN | |
| | | KLGSFGKTLGSKLKKNMGGLMHSK | |
| | | GSKPGGVGTGLGGSSGTETLEKKK | |
| | | KNSLKSWKGGKEEAAGDGPVSEKP | |
| | | PAESVGNGGSKYSQEVMQSLSILR | |
| | | TAMQGEGKFIFVGTLKMGHRHQYQ | |
| | | EEMIQRYLSDAEERFLAEQKQKEA | |
| | | ERKIMNGGIGGGPPPAKKPEPDAR | |
| | | EEQPTGPPAESRAMAFSTGYPGDF | |
| | | TIPRPSGGGVHCQEPRRQLAGGPC | |
| | | VGGLPPYATFPRQCPPGRPYPHQD | |
| | | SIPSLEPGSHSKDGLHRGALLPPP | |
| | | YRVADSYSNGYREPPEPDGWAGGL | |
| | | RGLPPTQTKCKQPNCSFYGHPETN | |
| | | NFCSCCYREELRRREREPDGELLV | |
| | | HRF* | |

TABLE 30
Catalytic Domains

| Gene | DNA | SEQ ID NO. | AA | SEQ ID NO. |
|---|---|---|---|---|
| ADS | ATGGCCCTGACCGAAGAGAAACCGAT | 292 | MALTEEKPIRPIANF | 293 |
| | CCGCCCGATCGCTAACTTCCCGCCGTC | | PPSIWGDQFLIYEK | |
| | TATCTGGGGTGACCAGTTCCTGATCTA | | QVEQGVEQIVNDL | |
| | CGAAAAGCAGGTTGAGCAGGGTGTTG | | KKEVRQLLKEALDI | |
| | AACAGATCGTAAACGACCTGAAGAAA | | PMKHANLLKLIDEI | |
| | GAAGTTCGTCAGCTGCTGAAAGAAGCT | | QRLGIPYHFEREID | |
| | CTGGACATCCCGATGAAACACGCTAAC | | HALQCIYETYGDN | |
| | CTGTTGAAGCTGATCGACGAGATCCAG | | WNGDRSSLWFRLM | |
| | CGTCTGGGTATCCCGTACCACTTCGAA | | RKQGYYVTCDVFN | |
| | CGCGAAATCGACCACGCACTGCAGTG | | NYKDKNGAFKQSL | |
| | CATCTACGAAACCTACGGCGACAACTG | | ANDVEGLLELYEA | |
| | GAACGGCGACCGTTCTTCTCTGTGGTT | | TSMRVPGEIILEDA | |
| | TCGTCTGATGCGTAAACAGGGCTACTA | | LGFTRSRLSIMTKD | |
| | CGTTACCTGTGACGTTTTTAACAACTA | | AFSTNPALFTEIQR | |
| | CAAGGACAAGAACGGTGCTTTCAAAC | | ALKQPLWKRLPRIE | |
| | AGTCTCTGGCTAACGACGTTGAAGGCC | | AAQYIPFYQQQDSH | |
| | TGCTGGAACTGTACGAAGCGACCTCCA | | NKTLLKLAKLEFNL | |
| | TGCGTGTACCGGGTGAAATCATCCTGG | | LQSLHKEELSHVCK | |
| | AGGACGCGCTGGGTTTCACCCGTTCTC | | WWKAFDIKKNAPC | |
| | GTCTGTCCATTATGACTAAAGACGCTT | | LRDRIVECYFWGL | |
| | TCTCTACTAACCCGGCTCTGTTCACCG | | GSGYEPQYSRARVF | |
| | AAATCCAGCGTGCTCTGAAACAGCCGC | | FTKAVAVITLIDDT | |
| | TGTGGAAACGTCTGCCGCGTATCGAAG | | YDAYGTYEELKIFT | |
| | CAGCACAGTACATTCCGTTTTACCAGC | | EAVERWSITCLDTL | |
| | AGCAGGACTCTCACAACAAGACCCTG | | PEYMKPIYKLFMDT | |
| | CTGAAACTGGCTAAGCTGGAATTCAAC | | YTEMEEFLAKEGR | |
| | CTGCTGCAGTCTCTGCACAAAGAAGAA | | TDLFNCGKEFVKEF | |
| | CTGTCTCACGTTTGTAAGTGGTGGAAG | | VRNLMVEAKWAN | |
| | GCATTTGACATCAAGAAAAACGCGCC | | EGHIPTTEEHDPVVI | |
| | GTGCCTGCGTGACCGTATCGTTGAATG | | ITGGANLLTTTCYL | |
| | TTACTTCTGGGGTCTGGGTTCTGGTTAT | | GMSDIFTKESVEW | |
| | GAACCACAGTACTCCCGTGCACGTGTG | | AVSAPPLFRYSGIL | |
| | TTCTTCACTAAAGCTGTAGCTGTTATC | | GRRLNDLMTHKAE | |
| | ACCCTGATCGATGACACTTACGATGCT | | QERKHSSSSLESYM | |
| | TACGGCACCTACGAAGAACTGAAGAT | | KEYNVNEEYAQTLI | |
| | CTTTACTGAAGCTGTAGAACGCTGGTC | | YKEVEDVWKDINR | |
| | TATCACTTGCCTGGACACTCTGCCGGA | | EYLTTKNIPRPLLM | |
| | GTACATGAAACCGATCTACAAACTGTT | | AVIYLCQFLEVQYA | |
| | CATGGATACCTACACCGAAATGGAGG | | GKDNFTRMGDEYK | |
| | AATTCCTGGCAAAAGAAGGCCGTACC | | HLIKSLLVYPMSI* | |
| | GACCTGTTCAACTGCGGTAAAGAGTTT | | | |
| | GTTAAAGAATTCGTACGTAACCTGATG | | | |
| | GTTGAAGCTAAATGGGCTAACGAAGG | | | |
| | CCATATCCCGACTACCGAAGAACATGA | | | |
| | CCCGGTTGTTATCATCACCGGCGGTGC | | | |
| | AAACCTGCTGACCACCACTTGCTATCT | | | |
| | GGGTATGTCCGACATCTTTACCAAGGA | | | |
| | ATCTGTTGAATGGGCTGTTTCTGCACC | | | |
| | GCCGCTGTTCCGTTACTCCGGTATTCT | | | |
| | GGGTCGTCGTCTGAACGACCTGATGAC | | | |
| | CCACAAAGCAGAGCAGGAACGTAAAC | | | |
| | ACTCTTCCTCCTCTCTGGAATCCTACAT | | | |
| | GAAGGAATATAACGTTAACGAGGAGT | | | |
| | ACGCACAGACTCTGATCTATAAAGAA | | | |
| | GTTGAAGACGTATGGAAAGACATCAA | | | |
| | CCGTGAATACCTGACTACTAAAAACAT | | | |
| | CCCGCGCCCGCTGCTGATGGCAGTAAT | | | |
| | CTACCTGTGCCAGTTCCTGGAAGTACA | | | |
| | GTACGCTGGTAAAGATAACTTCACTCG | | | |
| | CATGGGCGACGAATACAAACACCTGA | | | |
| | TCAAATCCCTGCTGGTTTACCCGATGT | | | |
| | CCATCTGA | | | |
| GHS | atgGCTCAAATCAGCGAATCAGTGTCTC | 294 | MAQISESVSPSTDL | 295 |
| | CAAGCACCGACCTTAAAAGCACGGAA | | KSTESSITSNRHGN | |
| | TCTTCTATTACCAGCAACCGCCACGGT | | MWEDDRIQSLNSP | |
| | AACATGTGGGAAGATGACCGCATTCA | | YGAPAYQERSEKLI | |
| | GAGCTTAAACAGCCCATATGGCGCACC | | EEIKLLFLSDMDDS | |
| | CGCTTATCAGGAACGTAGCGAAAAATT | | CNDSDRDLIKRLEI | |
| | GATTGAAGAAATTAAGCTCCTGTTTCT | | VDTVECLGIDRHFQ | |
| | GTCCGATATGGACGATAGTTGCAATGA | | PEIKLALDYVYRC | |
| | TTCGGATCGCGACTTGATCAAACGCCT | | WNERGIGEGSRDSL | |
| | GGAGATCGTAGATACGGTTGAGTGTCT | | KKDLNATALGFRA | |
| | GGGCATTGATCGTCATTTCCAACCTGA | | LRLHRYNVSSGVLE | |
| | AATTAAGCTGGCGCTGGATTACGTGTA | | NFRDDNGQFFCGST | |
| | CCGTTGCTGGAATGAGCGTGGCATCGG | | VEEEGAEAYNKHV | |
| | AGAAGGTAGCCGTGATAGCTTAAAAA | | RCMLSLSRASNILF | |
| | AGGACCTGAATGCGACCGCCTTGGGCT | | PGEKVMEEAKAFT | |
| | TTCGGGCTTTACGCTTACACCGTTATA | | TNYLKKVLAGREA | |
| | ATGTAAGCTCAGGAGTGCTGGAGAAC | | THVDESLLGEVKY | |
| | TTCCGTGATGACAATGGTCAATTCTTT | | ALEFPWHCSVQRW | |
| | TGCGGTTCTACTGTGGAGGAGGAAGG | | EARSFIEIFGQIDSEL | |
| | CGCGGAGGCCTACAATAAACATGTAC | | KSNLSKKMLELAK | |
| | GTTGCATGCTGTCCCTGTCCCGCGCTT | | LDFNILQCTHQKEL | |
| | CCAATATTTTATTCCCGGGCGAGAAAG | | QIISRWFADSSIASL | |
| | TGATGGAAGAAGCGAAGGCGTTTACG | | NFYRKCYVEFYFW | |
| | ACCAACTATCTTAAGAAAGTCCTGGCG | | MAAAISEPEFSGSR | |
| | GGTCGTGAAGCAACTCATGTCGACGA | | VAFTKIAILMTMLD | |
| | GAGTCTCCTTGGAGAGGTCAAGTATGC | | DLYDTHGTLDQLKI | |
| | ACTAGAATTTCCGTGGCATTGTTCCGT | | FTEGVRRWDVSLV | |
| | GCAGCGCTGGGAGGCACGTTCTTTTAT | | EGLPDFMKIAFEFW | |
| | CGAAATTTTCGGTCAGATTGATAGTGA | | LKTSNELIAEAVKA | |
| | ACTGAAAAGCAACCTCTCTAAAAAAA | | QGQDMAAYIRKNA | |
| | TGCTCGAACTCGCAAAACTTGATTTTA | | WERYLEAYLQDAE | |
| | ACATACTCCAGTGTACGCATCAAAAAG | | WIATGHVPTFDEYL | |
| | AGCTCCAGATCATTAGTCGATGGTTCG | | NNGTPNTGMCVLN | |
| | CCGATTCAAGTATCGCAAGTCTGAACT | | LIPLLLMGEHLPIDI | |
| | TTTACCGTAAATGCTATGTGGAATTTT | | LEQIFLPSRFHHLIE | |
| | ACTTCTGGATGGCCGCGGCAATTTCAG | | LASRLVDDARDFQ | |
| | AACCAGAATTTAGTGGCTCTCGCGTGG | | AEKDHGDLSCIECY | |
| | CATTCACTAAAATTGCGATCTTGATGA | | LKDHPESTVEDALN | |
| | CAATGTTAGATGACTTATACGACACGC | | HVNGLLGNCLLEM | |
| | ATGGGACGCTGGATCAATTGAAAATAT | | NWKFLKKQDSVPL | |
| | TTACCGAAGGTGTGCGCAGGTGGGAC | | SCKKYSFHVLARSI | |
| | GTGTCGCTGGTGGAGGGCCTGCCGGAT | | QFMYNQGDGFSISN | |
| | TTCATGAAAATTGCCTTTGAGTTCTGG | | KVIKDQVQKVLIVP | |
| | TTAAAGACCTCCAACGAACTGATTGCG | | VPI* | |
| | GAGGCGGTTAAGGCCCAAGGCCAGGA | | | |
| | TATGGCGGCCTATATCCGCAAAAACGC | | | |
| | TTGGGAACGCTATCTGGAAGCGTATTT | | | |
| | GCAGGATGCCGAATGGATCGCCACCG | | | |
| | GTCACGTTCCGACATTCGATGAATATC | | | |
| | TGAACAATGGCACCCCCAACACCGGT | | | |
| | ATGTGTGTACTTAATCTGATCCCGTTG | | | |
| | CTGCTTATGGGCGAACACTTGCCGATC | | | |
| | GATATTCTTGAACAGATCTTTCTGCCG | | | |
| | AGCCGGTTCCACCATCTGATTGAACTG | | | |
| | GCTAGCCGACTGGTCGATGATGCGAG | | | |
| | AGATTTTCAAGCCGAAAAAGATCATG | | | |
| | GTGATTTATCCTGCATCGAATGCTACC | | | |
| | TGAAAGACCATCCGGAATCAACAGTT | | | |
| | GAAGACGCCCTGAATCACGTCAACGG | | | |
| | CCTGCTGGGGAATTGTTTGCTGGAAAT | | | |
| | GAATTGGAAATTTCTGAAAAAACAGG | | | |
| | ACTCGGTACCTCTGTCGTGTAAAAAAT | | | |
| | ACTCATTCCACGTCCTGGCGCGGTCGA | | | |
| | TTCAGTTTATGTATAACCAGGGGGACG | | | |
| | GGTTTTCGATTTCGAACAAAGTTATTA | | | |
| | AAGACCAGGTCCAGAAAGTTCTAATC | | | |
| | GTTCCGGTTCCTATATAA | | | |
| TXS | ATGAGCAGCAGCACTGGCACTAGCAA | 296 | MSSSTGTSKVVSET | 297 |
| | GGTGGTTTCCGAGACTTCCAGTACCAT | | SSTIVDDIPRLSANY | |
| | TGTGGATGATATCCCTCGACTCTCCGC | | HGDLWHHNVIQTL | |
| | CAATTATCATGGCGATCTGTGGCACCA | | ETPFRESSTYQERA | |
| | CAATGTTATACAAACTCTGGAGACACC | | DELVVKIKDMFNA | |
| | GTTTCGTGAGAGTTCTACTTACCAAGA | | LGDGDISPSAYDTA | |
| | ACGGGCAGATGAGCTGGTTGTGAAAA | | WVARLATISSDGSE | |
| | TTAAAGATATGTTCAATGCGCTCGGAG | | KPRFPQALNWVEN | |
| | ACGGAGATATCAGTCCGTCTGCATACG | | NQLQDGSWGIESHF | |
| | ACACTGCGTGGGTGGCGAGGCTGGCG | | SLCDRLLNTTNSVI | |
| | ACCATTTCCTCTGATGGATCTGAGAAG | | ALSVWKTGHSQVQ | |
| | CCACGGTTTCCTCAGGCCCTCAACTGG | | QGAEFIAENLRLLN | |
| | GTTTTCAACAACCAGCTCCAGGATGGA | | EEDELSPDFQIIFPA | |
| | TCGTGGGGTATCGAATCGCACTTTAGT | | LLQKAKALGINLPY | |
| | TTATGCGATCGATTGCTTAACACGACC | | DLPFIKYLSTTREA | |
| | AATTCTGTTATCGCCCTCTCGGTTTGG | | RLTDVSAAADNIPA | |
| | AAAACAGGGCACAGCCAAGTACAACA | | NMLNALEGLEEVID | |
| | AGGTGCTGAGTTTATTGCAGAGAATCT | | WNKIMRFQSKDGS | |
| | AAGATTACTCAATGAGGAAGATGAGT | | FLSSPASTACVLMN | |
| | TGTCCCCGGATTTCCAAATAATCTTTC | | TGDEKCFTFLNNLL | |
| | CTGCTCTGCTGCAAAAGGCAAAAGCGT | | DKFGGCVPCMYSI | |
| | TGGGGATCAATCTTCCTTACGATCTTC | | DLLERLSLVDNIEH | |
| | CATTTATCAAATATTTGTCGACAACAC | | LGIGRHFKQEIKGA | |
| | GGGAAGCCAGGCTTACAGATGTTTCTG | | LDYVYRHWSERGI | |
| | CGGCAGCAGACAATATTCCAGCCAAC | | GWGRDSLVPDLNT | |
| | ATGTTGAATGCGTTGGAAGGACTCGAG | | TALGLRTLRMHGY | |
| | GAAGTTATTGACTGGAACAAGATTATG | | NVSSDVLNNFKDE | |
| | AGGTTTCAAAGTAAAGATGGATCTTTC | | NGRFFSSAGQTHVE | |
| | CTGAGCTCCCCTGCCTCCACTGCCTGT | | LRSVVNLFRASDLA | |
| | GTACTGATGAATACAGGGGACGAAAA | | FPDERAMDDARKF | |
| | ATGTTTCACTTTTCTCAACAATCTGCTC | | AEPYLREALATKIS | |
| | GACAAATTCGGCGGCTGCGTGCCCTGT | | TNTKLFKEIEYVVE | |
| | ATGTATTCCATCGATCTGCTGGAACGC | | YPWHMSIPRLEARS | |
| | CTTTCGCTGGTTGATAACATTGAGCAT | | YIDSYDDNYVWQR | |
| | CTCGGAATCGGTCGCCATTTCAAACAA | | KTLYRMPSLSNSKC | |
| | GAAATCAAAGGAGCTCTTGATTATGTC | | LELAKLDFNIVQSL | |
| | TACAGACATTGGAGTGAAAGGGGCAT | | HQEELKLLTRWWK | |
| | CGGTTGGGGCAGAGACAGCCTTGTTCC | | ESGMADINFTRHRV | |
| | AGATCTCAACACCACAGCCCTCGGCCT | | AEVYFSSATFEPEY | |
| | GCGAACTCTTCGCATGCACGGATACAA | | SATRIAFTKIGCLQ | |
| | TGTTTCTTCAGACGTTTTGAATAATTTC | | VLFDDMADIFATLD | |
| | AAAGATGAAAACGGGCGGTTCTTCTCC | | ELKSFTEGVKRWD | |
| | TCTGCGGGCCAAACCCATGTCGAATTG | | TSLLHEIPECMQTC | |
| | AGAAGCGTGGTGAATCTTTTCAGAGCT | | FKVWFKLMEEVNN | |
| | TCCGACCTTGCATTTCCTGACGAAAGA | | DVVKVQGRDMLA | |
| | GCTATGGACGATGCTAGAAAATTTGCA | | HIRKPWELYFNCY | |
| | GAACCATATCTTAGAGAGGCACTTGCA | | VQEREWLEAGYIPT | |
| | ACGAAAATCTCAACCAATACAAAACT | | FEEYLKTYAISVGL | |
| | ATTCAAAGAGATTGAGTACGTGGTGG | | GPCTLQPILLMGEL | |
| | AGTACCCTTGGCACATGAGTATCCCAC | | VKDDVVEKVHYPS | |
| | GCTTAGAAGCCAGAAGTTATATTGATT | | NMFELVSLSWRLT | |
| | CATATGACGACAATTATGTATGGCAGA | | NDTKTYQAEKARG | |
| | GGAAGACTCTATATAGAATGCCATCTT | | QQASGIACYMKDN | |
| | TGAGTAATTCAAAATGTTTAGAATTGG | | PGATEEDAIKHICR | |
| | CAAAATTGGACTTCAATATCGTACAAT | | VVDRALKEASFEYF | |
| | CTTTGCATCAAGAGGAGTTGAAGCTTC | | KPSNDIPMGCKSFIF | |
| | TAACAAGATGGTGGAAGGAATCCGGC | | NLRLCVQIFYKFID | |
| | ATGGCAGATATAAATTTCACTCGACAC | | GYGIANEEIKDYIR | |
| | CGAGTGGCGGAGGTTTATTTTTCATCA | | KVYIDPIQV* | |
| | GCTACATTTGAACCCGAATATTCTGCC | | | |
| | ACTAGAATTGCCTTCACAAAAATTGGT | | | |
| | TGTTTACAAGTCCTTTTTGATGATATG | | | |
| | GCTGACATCTTTGCAACACTAGATGAA | | | |
| | TTGAAAAGTTTCACTGAGGGAGTAAA | | | |
| | GAGATGGGATACATCTTTGCTACATGA | | | |
| | GATTCCAGAGTGTATGCAAACTTGCTT | | | |
| | TAAAGTTTGGTTCAAATTAATGGAAGA | | | |
| | AGTAAATAATGATGTGGTTAAGGTACA | | | |
| | AGGACGTGACATGCTCGCTCACATAAG | | | |
| | AAAACCCTGGGAGTTGTACTTCAATTG | | | |
| | TTATGTACAAGAAAGGGAGTGGCTTG | | | |
| | AAGCCGGGTATATACCAACTTTTGAAG | | | |
| | AGTACTTAAAGACTTATGCTATATCAG | | | |
| | TAGGCCTTGGACCGTGTACCCTACAAC | | | |
| | CAATACTACTAATGGGTGAGCTTGTGA | | | |
| | AAGATGATGTTGTTGAGAAAGTGCACT | | | |
| | ATCCCTCAAATATGTTTGAGCTTGTAT | | | |
| | CCTTGAGCTGGCGACTAACAAACGAC | | | |
| | ACCAAAACATATCAGGCTGAAAAGGC | | | |
| | TCGAGGACAACAAGCCTCAGGCATAG | | | |
| | CATGCTATATGAAGGATAATCCAGGA | | | |
| | GCAACTGAGGAAGATGCCATTAAGCA | | | |
| | CATATGTCGTGTTGTTGATCGGGCCTT | | | |
| | GAAAGAAGCAAGCTTTGAATATTTCAA | | | |
| | ACCATCCAATGATATCCCAATGGGTTG | | | |
| | CAAGTCCTTTATTTTTAACCTTAGATTG | | | |
| | TGTGTCCAAATCTTTTACAAGTTTATA | | | |
| | GATGGGTACGGAATCGCCAATGAGGA | | | |
| | GATTAAGGACTATATAAGAAAAGTTTA | | | |
| | TATTGATCCAATTCAAGTATGA | | | |

TABLE 31
Terpene Synthases

| Terpene | | | | |
|---|---|---|---|---|
| Synthase | DNA | SEQ ID NO. | AA | SEQ ID NO. |
| (S)-ß- | ATGGAGTTGGTAGACAC | 8 | MELVDTPSLEVFEDVV | 9 |
| Bisabolene | CCCTAGTCTTGAGGTATT | | VDRQVAGFDPSFWGD | |
| synthase | CGAGGATGTAGTTGTTGA | | YFITNQKSQSEAWMNE | |
| from | CCGTCAGGTAGCTGGCTT | | RAEELKNEVRSMFQN | |
| Zingiber | CGATCCGAGTTTCTGGGG | | VTGILQTMNLIDTIQLL | |
| officinale | CGATTATTTTATTACCAA | | GLDYHFMEEIAKALDH | |
| (D2YZP9) | CCAGAAGTCGCAGTCCG | | LKDVDMSKYGLYEVA | |
| | AAGCGTGGATGAACGAA | | LHFRLLRQKGFNISSD | |
| | CGCGCAGAAGAATTGAA | | VFKKYKDKEGKFMEE | |
| | AAACGAGGTCCGTAGCA | | LKDDAKGLLSLYNAA | |
| | TGTTCCAGAACGTAACTG | | YFGTKEETILDEAISFT | |
| | GAATCCTGCAAACGATG | | KDNLTSLLKDLNPPFA | |
| | AATCTTATCGACACGATT | | KLVSLTLKTPIQRSMK | |
| | CAACTTCTTGGACTTGAT | | RIFTRSYISIYQDEPTLN | |
| | TATCACTTCATGGAAGAA | | ETILELAKLDENMLQC | |
| | ATCGCAAAGGCCCTTGA | | LHQKELKKICAWWNN | |
| | CCACTTAAAGGATGTCG | | LNLDIMHLNFIRDRVV | |
| | ATATGAGTAAGTATGGCT | | ECYCWSMVIRHEPSCS | |
| | TATATGAAGTGGCCCTTC | | RARLISTKLLMLITVLD | |
| | ACTTTCGCCTTCTGCGCC | | DTYDSYSTLEESRLLT | |
| | AGAAGGGCTTTAACATC | | DAIQRWNPNEVDQLPE | |
| | AGTTCTGACGTATTTAAA | | YLRDFFLKMLNIFQEF | |
| | AAGTACAAAGATAAGGA | | ENELAPEEKFRILYLKE | |
| | AGGTAAATTTATGGAGG | | EWKIQSQSYFKECQW | |
| | AACTTAAGGATGACGCG | | RDDNYVPKLEEHMRL | |
| | AAAGGGTTGTTATCGTTA | | SIISVGFVLFYCGFLSG | |
| | TACAATGCAGCGTACTTT | | MEEAVATKDAFEWFA | |
| | GGTACGAAGGAAGAAAC | | SFPKIIEACATIIRITNDI | |
| | GATTTTGGATGAAGCAAT | | TSMEREQKRAHVAST | |
| | TTCTTTTACTAAAGACAA | | VDCYMKEYGTSKDVA | |
| | CTTAACATCTTTGTTAAA | | CEKLLGFVEDAWKTIN | |
| | GGATTTGAATCCACCATT | | EELLTETGLSREVIELS | |
| | CGCGAAACTTGTATCTCT | | FHSAQTTEFVYKHVDA | |
| | GACATTAAAGACTCCAA | | FTEPNTTMKENIFSLLV | |
| | TTCAACGTTCAATGAAGC | | HPIPI | |
| | GCATCTTTACACGCTCTT | | | |
| | ACATCTCCATTTACCAAG | | | |
| | ACGAGCCCACCTTGAAC | | | |
| | GAAACTATCTTAGAATTA | | | |
| | GCCAAGTTGGATTTCAAT | | | |
| | ATGTTACAATGTTTACAC | | | |
| | CAGAAGGAGTTGAAGAA | | | |
| | AATCTGTGCCTGGTGGAA | | | |
| | CAATCTGAACTTGGATAT | | | |
| | CATGCACTTGAATTTCAT | | | |
| | CCGCGACCGCGTGGTAG | | | |
| | AATGTTACTGCTGGAGTA | | | |
| | TGGTCATTCGTCACGAAC | | | |
| | CTTCCTGTTCTCGTGCCC | | | |
| | GCTTAATCAGCACGAAA | | | |
| | TTGCTGATGTTAATTACT | | | |
| | GTGCTTGACGATACGTAC | | | |
| | GATTCCTATAGTACTCTG | | | |
| | GAAGAGTCCCGCCTGCTT | | | |
| | ACAGACGCCATTCAACG | | | |
| | TTGGAATCCTAATGAGGT | | | |
| | GGACCAGTTACCAGAAT | | | |
| | ACCTTCGCGACTTCTTTC | | | |
| | TTAAGATGTTGAATATTT | | | |
| | TTCAGGAATTTGAGAAC | | | |
| | GAATTAGCTCCGGAGGA | | | |
| | GAAATTTCGTATTTTATA | | | |
| | CTTAAAGGAAGAATGGA | | | |
| | AGATCCAGTCCCAAAGTT | | | |
| | ACTTCAAAGAATGTCAGT | | | |
| | GGCGTGATGATAATTATG | | | |
| | TTCCAAAGCTGGAAGAA | | | |
| | CACATGCGTTTATCGATT | | | |
| | ATTAGTGTAGGCTTCGTT | | | |
| | TTATTCTACTGTGGCTTT | | | |
| | TTATCAGGTATGGAGGA | | | |
| | GGCCGTTGCAACGAAAG | | | |
| | ACGCCTTTGAATGGTTTG | | | |
| | CGTCCTTTCCAAAAATTA | | | |
| | TTGAAGCTTGTGCTACAA | | | |
| | TTATTCGTATCACCAATG | | | |
| | ATATCACGTCCATGGAAC | | | |
| | GTGAACAAAAACGCGCA | | | |
| | CATGTGGCCTCAACTGTA | | | |
| | GACTGTTATATGAAGGA | | | |
| | ATACGGAACGTCAAAAG | | | |
| | ACGTCGCGTGCGAAAAA | | | |
| | CTGTTGGGCTTCGTGGAG | | | |
| | GACGCATGGAAGACGAT | | | |
| | CAATGAAGAGCTTCTTAC | | | |
| | TGAGACTGGGCTTTCACG | | | |
| | CGAAGTCATTGAACTTTC | | | |
| | TTTCCATTCTGCACAGAC | | | |
| | TACGGAGTTCGTATATAA | | | |
| | GCACGTTGATGCGTTCAC | | | |
| | CGAGCCGAATACTACTAT | | | |
| | GAAAGAAAACATCTTCT | | | |
| | CGTTATTGGTACATCCCA | | | |
| | TCCCCATTTAA | | | |
| ß-Bisabolene | ATGGATGCCTTCGCTACG | 10 | MDAFATSPTSALIKAV | 11 |
| synthase | TCTCCTACGTCAGCCTTA | | NCIAHVTPMAGEDSSE | |
| from | ATCAAAGCAGTAAACTG | | NRRASNYKPSTWDYE | |
| Santalum | CATCGCCCACGTGACGCC | | FLQSLATSHNTVQEKH | |
| austrocaledonicum | TATGGCTGGCGAGGATTC | | MKMAEKLKEEVKSMI | |
| (E3W205) | CTCCGAGAACCGTCGTGC | | KGQMEPVAKLELINIV | |
| | GTCAAATTACAAACCATC | | QRLGLKYRFESEIKEEL | |
| | AACATGGGACTACGAGT | | FSLYKDGTDAWWVDN | |
| | TCTTGCAGAGTTTGGCTA | | LHATALRFRLLRENGIF | |
| | CCTCGCATAATACCGTAC | | VPQDVFETFKDKSGKF | |
| | AGGAAAAGCACATGAAA | | KSQLCKDVRGLLSLYE | |
| | ATGGCGGAGAAGTTGAA | | ASYLGWEGEDLLDEA | |
| | AGAAGAAGTCAAGAGTA | | KKFSTTNLNNVKESISS | |
| | TGATCAAAGGCCAGATG | | NTLGRLVKHALNLPLH | |
| | GAACCTGTGGCGAAGCT | | WSAARYEARWFIDEY | |
| | TGAACTGATTAACATCGT | | EKEENVNPNLLKYAKL | |
| | CCAGCGTTTGGGTCTGAA | | DFNIVQSIHQGELGNL | |
| | ATATCGTTTTGAATCTGA | | ARWWVETGLDKLSFV | |
| | AATTAAAGAAGAGCTGT | | RNTLMQNFMWGCAM | |
| | TCTCTTTGTACAAGGACG | | VFEPQYGKVRDAAVK | |
| | GTACCGATGCATGGTGG | | QASLIAMVDDVYDVY | |
| | GTCGATAACTTGCACGCA | | GSLEELEIFTDIVDRWD | |
| | ACCGCGCTGCGTTTTCGT | | ITGIDKLPRNISMILLT | |
| | TTACTGCGCGAGAACGG | | MFNTANQIGYDLLRDR | |
| | GATCTTCGTCCCGCAGGA | | GENGIPHIAQAWATLC | |
| | CGTGTTCGAAACATTTAA | | KKYLKEAKWYHSGYK | |
| | AGACAAGTCCGGAAAGT | | PTLEEYLENGLVSISFV | |
| | TCAAGTCACAATTGTGCA | | LSLVTAYLQTETLENL | |
| | AGGATGTGCGTGGGTTAT | | TYESAAYVNSVPPLVR | |
| | TGAGTTTGTATGAAGCTT | | YSGLLNRLYNDLGTSS | |
| | CTTACTTAGGCTGGGAAG | | AEIARGDTLKSIQCYM | |
| | GCGAAGATCTTCTTGACG | | TQTGATEEAAREHIKG | |
| | AAGCTAAGAAGTTTAGC | | LVHEAWKGMNKCLFE | |
| | ACTACGAACTTGAACAA | | QTPFAEPFVGFNVNTV | |
| | CGTGAAGGAATCGATCT | | RGSQFFYQHGDGYAV | |
| | CGTCAAATACGCTTGGCC | | TESWTKDLSLSVLIHPI | |
| | GTTTAGTCAAACATGCCT | | PLNEED | |
| | TAAATTTGCCGTTACACT | | | |
| | GGTCTGCTGCCCGTTACG | | | |
| | AAGCACGCTGGTTTATCG | | | |
| | ACGAATACGAAAAGGAA | | | |
| | GAAAACGTGAATCCCAA | | | |
| | CTTGCTTAAGTACGCTAA | | | |
| | GTTAGATTTCAACATCGT | | | |
| | CCAAAGCATCCATCAGG | | | |
| | GTGAGCTGGGCAATTTA | | | |
| | GCTCGTTGGTGGGTTGAG | | | |
| | ACAGGTCTGGACAAGTT | | | |
| | GTCTTTCGTGCGTAATAC | | | |
| | GCTTATGCAGAACTTTAT | | | |
| | GTGGGGATGTGCTATGGT | | | |
| | ATTCGAACCCCAGTACG | | | |
| | GCAAGGTACGCGATGCT | | | |
| | GCTGTTAAGCAGGCCAG | | | |
| | CTTAATTGCAATGGTAGA | | | |
| | TGACGTATATGACGTTTA | | | |
| | CGGGTCGCTGGAGGAAC | | | |
| | TTGAGATCTTTACCGATA | | | |
| | TCGTCGATCGTTGGGACA | | | |
| | TTACCGGCATTGACAAAC | | | |
| | TTCCTCGCAATATCAGCA | | | |
| | TGATTTTATTAACAATGT | | | |
| | TCAATACGGCGAACCAA | | | |
| | ATCGGGTATGATTTGCTT | | | |
| | CGTGACCGTGGTTTTAAT | | | |
| | GGTATCCCGCATATTGCG | | | |
| | CAAGCTTGGGCAACTCTG | | | |
| | TGTAAAAAGTACCTTAA | | | |
| | AGAAGCTAAATGGTACC | | | |
| | ACTCCGGATACAAGCCG | | | |
| | ACCTTAGAAGAGTACCT | | | |
| | GGAAAACGGGTTGGTGT | | | |
| | CGATTTCCTTTGTGCTGT | | | |
| | CACTGGTGACTGCTTATC | | | |
| | TGCAGACTGAGACATTG | | | |
| | GAAAATTTGACCTACGA | | | |
| | GTCGGCCGCGTATGTTAA | | | |
| | CTCCGTACCCCCGTTGGT | | | |
| | ACGCTATAGTGGATTATT | | | |
| | AAATCGTTTATATAACGA | | | |
| | TCTGGGTACGTCATCCGC | | | |
| | TGAGATTGCCCGCGGGG | | | |
| | ACACCTTAAAGTCTATCC | | | |
| | AATGCTACATGACTCAG | | | |
| | ACGGGGGCCACGGAAGA | | | |
| | GGCGGCGCGCGAACACA | | | |
| | TCAAAGGGTTAGTACAT | | | |
| | GAGGCATGGAAGGGTAT | | | |
| | GAATAAGTGTCTTTTCGA | | | |
| | ACAAACCCCATTTGCTGA | | | |
| | GCCATTCGTCGGTTTCAA | | | |
| | CGTTAATACGGTGCGCG | | | |
| | GGTCCCAGTTCTTTTATC | | | |
| | AGCATGGTGATGGGTAC | | | |
| | GCCGTCACAGAAAGTTG | | | |
| | GACCAAAGACCTGTCCCT | | | |
| | TTCAGTTTTAATTCATCC | | | |
| | AATTCCCTTGAATGAGGA | | | |
| | GGACTAA | | | |
| Taxadiene | ATGAGCAGCAGCACTGG | 12 | MAQLSFNAALKMNAL | 13 |
| synthase | CACTAGCAAGGTGGTTTC | | GNKAIHDPTNCRAKSE | |
| from | CGAGACTTCCAGTACCAT | | RQMMWVCSRSGRTRV | |
| Taxus brevifola | TGTGGATGATATCCCTCG | | KMSRGSGGPGPVVMM | |
| (Q41594) | ACTCTCCGCCAATTATCA | | SSSTGTSKVVSETSSTI | |
| | TGGCGATCTGTGGCACCA | | VDDIPRLSANYHGDL | |
| | CAATGTTATACAAACTCT | | WHHNVIQTLETPFRES | |
| | GGAGACACCGTTTCGTG | | STYQERADELVVKIKD | |
| | AGAGTTCTACTTACCAAG | | MFNALGDGDISPSAYD | |
| | AACGGGCAGATGAGCTG | | TAWVARLATISSDGSE | |
| | GTTGTGAAAATTAAAGA | | KPRFPQALNWVENNQ | |
| | TATGTTCAATGCGCTCGG | | LQDGSWGIESHFSLCD | |
| | AGACGGAGATATCAGTC | | RLLNTTNSVIALSVWK | |
| | CGTCTGCATACGACACTG | | TGHSQVQQGAEFIAEN | |
| | CGTGGGTGGCGAGGCTG | | LRLLNEEDELSPDFQIIF | |
| | GCGACCATTTCCTCTGAT | | PALLQKAKALGINLPY | |
| | GGATCTGAGAAGCCACG | | DLPFIKYLSTTREARLT | |
| | GTTTCCTCAGGCCCTCAA | | DVSAAADNIPANMLN | |
| | CTGGGTTTTCAACAACCA | | ALEGLEEVIDWNKIMR | |
| | GCTCCAGGATGGATCGT | | FQSKDGSFLSSPASTAC | |
| | GGGGTATCGAATCGCAC | | VLMNTGDEKCFTFLN | |
| | TTTAGTTTATGCGATCGA | | NLLDKFGGCVPCMYSI | |
| | TTGCTTAACACGACCAAT | | DLLERLSLVDNIEHLGI | |
| | TCTGTTATCGCCCTCTCG | | GRHFKQEIKGALDYVY | |
| | GTTTGGAAAACAGGGCA | | RHWSERGIGWGRDSL | |
| | CAGCCAAGTACAACAAG | | VPDLNTTALGLRTLRM | |
| | GTGCTGAGTTTATTGCAG | | HGYNVSSDVLNNFKD | |
| | AGAATCTAAGATTACTCA | | ENGRFFSSAGQTHVEL | |
| | ATGAGGAAGATGAGTTG | | RSVVNLFRASDLAFPD | |
| | TCCCCGGATTTCCAAATA | | ERAMDDARKFAEPYL | |
| | ATCTTTCCTGCTCTGCTG | | REALATKISTNTKLFKE | |
| | CAAAAGGCAAAAGCGTT | | IEYVVEYPWHMSIPRL | |
| | GGGGATCAATCTTCCTTA | | EARSYIDSYDDNYVW | |
| | CGATCTTCCATTTATCAA | | QRKTLYRMPSLSNSKC | |
| | ATATTTGTCGACAACACG | | LELAKLDFNIVQSLHQ | |
| | GGAAGCCAGGCTTACAG | | EELKLLTRWWKESGM | |
| | ATGTTTCTGCGGCAGCAG | | ADINFTRHRVAEVYFS | |
| | ACAATATTCCAGCCAAC | | SATFEPEYSATRIAFTK | |
| | ATGTTGAATGCGTTGGAA | | IGCLQVLFDDMADIFA | |
| | GGACTCGAGGAAGTTAT | | TLDELKSFTEGVKRWD | |
| | TGACTGGAACAAGATTA | | TSLLHEIPECMQTCFK | |
| | TGAGGTTTCAAAGTAAA | | VWFKLMEEVNNDVVK | |
| | GATGGATCTTTCCTGAGC | | VQGRDMLAHIRKPWE | |
| | TCCCCTGCCTCCACTGCC | | LYFNCYVQEREWLEA | |
| | TGTGTACTGATGAATACA | | GYIPTFEEYLKTYAISV | |
| | GGGGACGAAAAATGTTT | | GLGPCTLQPILLMGEL | |
| | CACTTTTCTCAACAATCT | | VKDDVVEKVHYPSNM | |
| | GCTCGACAAATTCGGCG | | FELVSLSWRLINDTKT | |
| | GCTGCGTGCCCTGTATGT | | YQAEKARGQQASGIA | |
| | ATTCCATCGATCTGCTGG | | CYMKDNPGATEEDAI | |
| | AACGCCTTTCGCTGGTTG | | KHICRVVDRALKEASF | |
| | ATAACATTGAGCATCTCG | | EYFKPSNDIPMGCKSFI | |
| | GAATCGGTCGCCATTTCA | | FNLRLCVQIFYKFIDGY | |
| | AACAAGAAATCAAAGGA | | GIANEEIKDYIRKVYID | |
| | GCTCTTGATTATGTCTAC | | PIQV | |
| | AGACATTGGAGTGAAAG | | | |
| | GGGCATCGGTTGGGGCA | | | |
| | GAGACAGCCTTGTTCCAG | | | |
| | ATCTCAACACCACAGCCC | | | |
| | TCGGCCTGCGAACTCTTC | | | |
| | GCATGCACGGATACAAT | | | |
| | GTTTCTTCAGACGTTTTG | | | |
| | AATAATTTCAAAGATGA | | | |
| | AAACGGGCGGTTCTTCTC | | | |
| | CTCTGCGGGCCAAACCC | | | |
| | ATGTCGAATTGAGAAGC | | | |
| | GTGGTGAATCTTTTCAGA | | | |
| | GCTTCCGACCTTGCATTT | | | |
| | CCTGACGAAAGAGCTAT | | | |
| | GGACGATGCTAGAAAAT | | | |
| | TTGCAGAACCATATCTTA | | | |
| | GAGAGGCACTTGCAACG | | | |
| | AAAATCTCAACCAATAC | | | |
| | AAAACTATTCAAAGAGA | | | |
| | TTGAGTACGTGGTGGAGT | | | |
| | ACCCTTGGCACATGAGTA | | | |
| | TCCCACGCTTAGAAGCCA | | | |
| | GAAGTTATATTGATTCAT | | | |
| | ATGACGACAATTATGTAT | | | |
| | GGCAGAGGAAGACTCTA | | | |
| | TATAGAATGCCATCTTTG | | | |
| | AGTAATTCAAAATGTTTA | | | |
| | GAATTGGCAAAATTGGA | | | |
| | CTTCAATATCGTACAATC | | | |
| | TTTGCATCAAGAGGAGTT | | | |
| | GAAGCTTCTAACAAGAT | | | |
| | GGTGGAAGGAATCCGGC | | | |
| | ATGGCAGATATAAATTTC | | | |
| | ACTCGACACCGAGTGGC | | | |
| | GGAGGTTTATTTTTCATC | | | |
| | AGCTACATTTGAACCCGA | | | |
| | ATATTCTGCCACTAGAAT | | | |
| | TGCCTTCACAAAAATTGG | | | |
| | TTGTTTACAAGTCCTTTT | | | |
| | TGATGATATGGCTGACAT | | | |
| | CTTTGCAACACTAGATGA | | | |
| | ATTGAAAAGTTTCACTGA | | | |
| | GGGAGTAAAGAGATGGG | | | |
| | ATACATCTTTGCTACATG | | | |
| | AGATTCCAGAGTGTATGC | | | |
| | AAACTTGCTTTAAAGTTT | | | |
| | GGTTCAAATTAATGGAA | | | |
| | GAAGTAAATAATGATGT | | | |
| | GGTTAAGGTACAAGGAC | | | |
| | GTGACATGCTCGCTCACA | | | |
| | TAAGAAAACCCTGGGAG | | | |
| | TTGTACTTCAATTGTTAT | | | |
| | GTACAAGAAAGGGAGTG | | | |
| | GCTTGAAGCCGGGTATAT | | | |
| | ACCAACTTTTGAAGAGTA | | | |
| | CTTAAAGACTTATGCTAT | | | |
| | ATCAGTAGGCCTTGGACC | | | |
| | GTGTACCCTACAACCAAT | | | |
| | ACTACTAATGGGTGAGCT | | | |
| | TGTGAAAGATGATGTTGT | | | |
| | TGAGAAAGTGCACTATC | | | |
| | CCTCAAATATGTTTGAGC | | | |
| | TTGTATCCTTGAGCTGGC | | | |
| | GACTAACAAACGACACC | | | |
| | AAAACATATCAGGCTGA | | | |
| | AAAGGCTCGAGGACAAC | | | |
| | AAGCCTCAGGCATAGCA | | | |
| | TGCTATATGAAGGATAAT | | | |
| | CCAGGAGCAACTGAGGA | | | |
| | AGATGCCATTAAGCACA | | | |
| | TATGTCGTGTTGTTGATC | | | |
| | GGGCCTTGAAAGAAGCA | | | |
| | AGCTTTGAATATTTCAAA | | | |
| | CCATCCAATGATATCCCA | | | |
| | ATGGGTTGCAAGTCCTTT | | | |
| | ATTTTTAACCTTAGATTG | | | |
| | TGTGTCCAAATCTTTTAC | | | |
| | AAGTTTATAGATGGGTAC | | | |
| | GGAATCGCCAATGAGGA | | | |
| | GATTAAGGACTATATAA | | | |
| | GAAAAGTTTATATTGATC | | | |
| | CAATTCAAGTATGA | | | |
| Terpene | ATGAGCAACTTCCTGGTG | 14 | MSTSSVSISLSSLVIDE | 15 |
| synthase | AGCACTTGTAGTAGCCCA | | NNSTKQDHVIRNTVTF | |
| from Cynara | TTAGCGCTGGATGAAAA | | HPSIWGDQFLVYDEKD | |
| cardunculus | CAATTCGACGAAACAGG | | DLVAEKQLVEELTEEI | |
| var. | ACCATGTGATCCGTAATA | | RKKLFITASSIHEPLQQI | |
| scolymus | CCGTTACGTTTCATAGTT | | QLIDAIQRLGVAYHFE | |
| (A0A118JXI9) | CTATATGGGGTGATCAAT | | KEIEEALQHVYRTYGH | |
| | TTCTTACCTATGATGAGA | | QGIHNNNNLQSVSLWF | |
| | AAGACGATCTGGTGGCG | | RILRQQGFNVSPEIFKN | |
| | GAAAAGCAATTGGCCGA | | HMDEKGNLLSNDVES | |
| | AGAGCTGATCGAAGAGA | | MLALYEASYMRVEGE | |
| | CACGCAAGGAATTAATT | | KVLDDALKFTKTHLAI | |
| | ATCACCACCTCTAGCCAT | | IAQHPSCDSSLRTQIQE | |
| | GAACCAATCCAACATAT | | ALRQPLRKRLPRLEAV | |
| | GAAACTTATCCAGCTGAT | | RYIPIYQQQSSHNQLLL | |
| | CGATGCTGTCCAGCGATT | | KLAKLDFNMLQSMHK | |
| | AGGAGTTGCCTATCACTT | | KELSQICKWWKDLDM | |
| | TGAGAAAGAGATAGAAG | | QNKLPFVRDRLIEGYF | |
| | ATGCCCTTCAACACGTAT | | CILGIYFEPHHSRLRMF | |
| | ACCGTACATATGGCCACC | | LIKSCMWLIVMDDTFD | |
| | AGGGTATACACAACAAC | | NYGTYEELKIFTEVVE | |
| | AATGACCTCCAGTCCATT | | RWSISCLDLLPEYMKV | |
| | TCACTCTGGTTCCGCATA | | IYLELVNIHQEMEESLE | |
| | CTGAGACAACAGGGTTT | | KEGKTYHIYYVKEMA | |
| | CAATGTGAGCAGCGAAA | | KEYTRSLLAEAKWLK | |
| | TCTTTAAGAACCATATGG | | DGYMPTLDEYISNSLIT | |
| | ATGAAAAAGGCAACCTG | | TTYAVVIEGSYVGGPD | |
| | TTTAGCAATGATGTGCAG | | MLVTEDSFKWVATHP | |
| | TCGATGCTGGCACTCTAT | | PLVKASCLILRLMNDI | |
| | GAGGCCTCCTATATGCGT | | ATHKEEQERSHVASSI | |
| | GTGGAGGGCGAGAAGGT | | ECYIKETGATEEEACE | |
| | GCTTGATGATGCACTGGA | | YFSKQVEDAWKVINR | |
| | GTTTACCAAGACACACCT | | DSLKPTDVPFPLVKPVI | |
| | CGCAATTATTGCAAAAG | | NLARISDVVYKGSING | |
| | ACCCGTCATGTGATTCTT | | YNHAGKELIQNIKSLL | |
| | CATTGCGGACCCAGATTC | | VHPLI | |
| | AAGACGCGCTGAGACAG | | | |
| | CCACTGAGAAAACGACT | | | |
| | CCCGCGTTTGGAGGCCGT | | | |
| | GCGCTACATTCCGATATA | | | |
| | TCAGCAGCAGAGTAGCC | | | |
| | ATAATCAAATTCTGCTGA | | | |
| | AATTGGCCAAATTAGATT | | | |
| | TCAACATGCTGCAGACG | | | |
| | ATGCACAAAAAGGAACT | | | |
| | TAGCGAGATTTGTAAATG | | | |
| | GTGGAAAGATCTCGATA | | | |
| | TGCAGAACAAACTGCCG | | | |
| | TTTGTTCGCGATCGTTTG | | | |
| | ATTGAAGGCTACTTTTGG | | | |
| | ATACTGGGTATCTATTTC | | | |
| | GAACCTCATCATTCCCGA | | | |
| | TCACGTATGTTTCTCATA | | | |
| | AAGAGCTGTATGTGGCTT | | | |
| | GTAGTTATCGATGACACC | | | |
| | TTTGACAATTACGGCACC | | | |
| | TATGAAGAGCTCGAAAT | | | |
| | ATTTACTGAAGCCGTTGA | | | |
| | GCGTTGGTCGATTAATTG | | | |
| | CCTGGATATGTTGCCTGA | | | |
| | GTATATGAAACTGATTTA | | | |
| | CAAAGAGCTTGTGATTGT | | | |
| | TCACCAGGAAATGGAGG | | | |
| | AAACGTTAGAAAAAGAA | | | |
| | GGGAAAGCTTATCACATT | | | |
| | CATCATGTGAAGGAGCT | | | |
| | CGCTAAGGAGTGCACCC | | | |
| | GAAGCTTGCTCGTCGAA | | | |
| | GCAAAATGGCTTAAAGA | | | |
| | AGGGTATATGCCGACATT | | | |
| | GGACGAGTATATTAGTA | | | |
| | ATTCTTTGATTACGTGTG | | | |
| | CTTATGCGGTTATGATTG | | | |
| | CCCGGAGCTACGTAGGC | | | |
| | GGTGACGATAAATTAGTT | | | |
| | AATGAAGACTCCTTCAA | | | |
| | GTGGGTTGCGACCCACCC | | | |
| | GCCACTGGTGAAAGCGT | | | |
| | CCTGTTTGATTCTTCGTC | | | |
| | TGATGGATGATATTGCGA | | | |
| | CCCATAAAGAGGAACAG | | | |
| | GAGCGTGGCCATGTGGC | | | |
| | AAGCAGTATTGAATGCT | | | |
| | ATATTAAAGAGACAGGT | | | |
| | GCTACAGAGGAAGAGGC | | | |
| | CCGTGAACACTTTAGTAA | | | |
| | ACAGGTGGAAGATGCCT | | | |
| | GGAAAGTTGTTAATCGTG | | | |
| | AGAGCCTTCGTCCGACCG | | | |
| | CGGTTGCCTTTCCGCTCG | | | |
| | TCATGCCGGCGATAAATC | | | |
| | TCGCCCGCATGTGCGATG | | | |
| | CGTTATATAAGGGTAACC | | | |
| | ATGATGGATATAATCAC | | | |
| | GCGGGTAAGGAAGTGAT | | | |
| | CCAGTATATAAAATCTCT | | | |
| | GCTCGTACATCCTTTGAT | | | |
| | CTAA | | | |
| (+)-α- | ATGTCACTTACTGAGGAA | 16 | MSLTEEKPIRPIANFSP | 17 |
| Bisabolol | AAGCCCATTCGCCCAATC | | SIWEDQFLIYAKQVEH | |
| synthase | GCGAATTTTAGCCCCAGT | | GVEQRVKDLTKEVRQ | |
| from | ATTTGGGAAGACCAGTTC | | LLKEALDIPMKHANLL | |
| Artemisia | TTGATCTATGCTAAGCAG | | KLIDEIQRLGISYLFEQ | |
| kurramensis | GTTGAGCATGGCGTGGA | | EIDHALQHIYETYGDN | |
| (A0A1L7NYG3) | GCAGCGTGTTAAGGACC | | WSGDRSSLWFRLMRK | |
| | TCACAAAGGAAGTGCGT | | QGYFVTCDVFNNHKD | |
| | CAGCTGCTCAAAGAGGC | | ESGAFKQSLANDVEGL | |
| | GCTCGACATCCCGATGA | | LELYEATSMRVAGEIIL | |
| | AGCACGCGAATTTATTGA | | DDALVFTRSNLSIIAKD | |
| | AGTTGATCGACGAGATC | | TLSTNPALSTEIQRALK | |
| | CAGCGTTTGGGCATCAGC | | QPLWKRLPRIEAAQYI | |
| | TACTTATTTGAACAAGAA | | PFYEQQDSHNMALLK | |
| | ATCGACCATGCTCTCCAG | | LAKLEFNLLQSLHREE | |
| | CATATTTACGAGACCTAC | | LSQLSKWWKAFDVKN | |
| | GGCGACAACTGGTCGGG | | NAPYSRDRIVECYFWG | |
| | TGATCGCAGCTCACTGTG | | LASRFEPQFSRARIFLA | |
| | GTTCCGTCTGATGCGTAA | | KVIALVTLIDDTYDAY | |
| | GCAGGGCTACTTTGTAAC | | GTYEELKIFTEAIERWS | |
| | GTGTGACGTCTTCAACAA | | ITCLDMIPEYMKPIYKL | |
| | CCACAAGGATGAGAGCG | | LMDTYTEMEEVLAKE | |
| | GAGCGTTCAAGCAAAGC | | GKTDIFDCGKEFVKDF | |
| | CTTGCCAACGACGTCGA | | VRVLMVEAQWLNEGH | |
| | AGGTTTACTTGAGTTGTA | | IPTTEELDSIAVNLGGA | |
| | TGAGGCAACCTCTATGCG | | NLLTTTCYLGMSDIVT | |
| | TGTAGCAGGAGAAATCA | | KEAVEWAVSEPPLLRY | |
| | TCCTTGACGACGCTTTGG | | KGILGRRLNDLAGHKE | |
| | TTTTCACTCGCAGTAACT | | EQERKHVSSSVESYMK | |
| | TGTCTATTATCGCGAAAG | | EYNVSEEYAQNLLYK | |
| | ACACGCTTAGTACTAATC | | QVEDLWKDINREYLIT | |
| | CCGCTTTGAGTACCGAGA | | KTIPRPLLVAVINLVHF | |
| | TTCAGCGCGCATTGAAAC | | LEVLYAEKDNFTRMG | |
| | AACCGCTTTGGAAGCGTC | | DEYKDLVKSLLVYPM | |
| | TCCCGCGCATTGAAGCTG | | SI | |
| | CTCAATACATTCCTTTCT | | | |
| | ATGAACAACAGGATAGC | | | |
| | CATAATATGGCGCTGCTT | | | |
| | AAGTTGGCAAAGTTAGA | | | |
| | ATTCAACCTTCTGCAATC | | | |
| | GCTGCATCGCGAAGAGC | | | |
| | TTTCCCAATTGTCTAAGT | | | |
| | GGTGGAAAGCTTTCGAT | | | |
| | GTAAAGAACAATGCACC | | | |
| | GTACAGTCGCGATCGCAT | | | |
| | CGTGGAGTGTTATTTCTG | | | |
| | GGGCTTAGCTAGCCGTTT | | | |
| | TGAACCCCAATTCTCTCG | | | |
| | CGCCCGTATCTTCCTTGC | | | |
| | AAAAGTAATTGCCTTGGT | | | |
| | CACGTTAATTGACGATAC | | | |
| | CTATGACGCATATGGCAC | | | |
| | GTATGAAGAACTCAAGA | | | |
| | TTTTCACTGAAGCGATCG | | | |
| | AGCGCTGGAGCATCACA | | | |
| | TGTTTGGATATGATCCCT | | | |
| | GAGTACATGAAGCCTATT | | | |
| | TACAAGCTGTTAATGGAC | | | |
| | ACTTATACGGAGATGGA | | | |
| | AGAGGTTCTTGCGAAGG | | | |
| | AAGGGAAGACGGATATC | | | |
| | TTTGACTGCGGCAAGGA | | | |
| | GTTTGTGAAGGACTTTGT | | | |
| | CCGTGTACTTATGGTAGA | | | |
| | GGCCCAGTGGCTTAACG | | | |
| | AAGGCCACATTCCCACG | | | |
| | ACCGAGGAATTAGATTCT | | | |
| | ATCGCGGTGAACCTCGGT | | | |
| | GGCGCGAATTTATTAACG | | | |
| | ACTACCTGCTATCTGGGT | | | |
| | ATGTCTGACATCGTCACA | | | |
| | AAAGAAGCGGTCGAATG | 18 | | 19 |
| | GGCTGTGAGTGAGCCGC | | | |
| | CTCTGTTACGTTATAAGG | | | |
| | GTATTCTTGGTCGTCGTT | | | |
| | TAAATGACCTTGCCGGGC | | | |
| | ACAAGGAAGAGCAGGAA | | | |
| | CGTAAACACGTGTCGTCG | | | |
| | TCTGTCGAGAGTTACATG | | | |
| | AAGGAGTATAATGTGTC | | | |
| | CGAGGAATACGCTCAAA | | | |
| | ATCTGCTTTATAAACAGG | | | |
| | TTGAGGATCTGTGGAAG | | | |
| | GACATTAACCGTGAGTAT | | | |
| | TTGATCACTAAGACAATC | | | |
| | CCCCGTCCTTTACTGGTA | | | |
| | GCGGTGATCAATCTCGTG | | | |
| | CACTTTCTGGAAGTCCTG | | | |
| | TACGCGGAGAAGGACAA | | | |
| | CTTTACTCGCATGGGTGA | | | |
| | CGAGTATAAGGACCTGG | | | |
| | TGAAGTCATTACTCGTCT | | | |
| | ACCCTATGAGTATCTGA | | | |
| (+)-epi-α- | ATGAATAGTACCAGCCG | 18 | MNSTSRRSANYKPTIW | 19 |
| Bisabolol | TCGTTCAGCAAACTATAA | | NNEYLQSLNSIYGEKR | |
| synthase | ACCGACCATTTGGAATA | | FLEQAEKLKDEVRMLL | |
| from Phyla | ATGAATACCTGCAGTCAC | | EKTSDPLDHIELVDVL | |
| dulcis | TGAACAGCATCTATGGA | | QRLAISYHFTEYIDRNL | |
| (J7LH11) | GAAAAGAGATTCCTTGA | | KNIYDILIDGRRWNHA | |
| | ACAGGCCGAAAAACTGA | | DNLHATTLSFRLLRQH | |
| | AAGATGAAGTTCGCATG | | GYQVSPEVFRNFMDET | |
| | CTGCTGGAAAAAACCTCT | | GNFKKNLCDDIKGLLS | |
| | GACCCGCTGGACCACAT | | LYEASYLLTEGETIMD | |
| | CGAACTTGTTGACGTCCT | | SAQAFATHHLKQKLEE | |
| | GCAACGCCTTGCAATATC | | NMNKNLGDEIAHALE | |
| | GTACCATTTTACTGAATA | | LPLHWRVPKLDVRWSI | |
| | TATCGATCGGAATTTGAA | | DAYERRQDMNPLLLE | |
| | AAATATTTACGATATACT | | LAKLDFNIAQSMYQDE | |
| | CATCGACGGGCGGCGGT | | LKELSRWYSKTHLPEK | |
| | GGAATCACGCGGATAAC | | LAFARDRLVESYLWG | |
| | CTGCATGCCACGACTCTC | | LGLASEPHHKYCRMM | |
| | TCCTTTAGACTTTTACGT | | VAQSTTLISIIDDIYDV | |
| | CAGCATGGTTACCAAGTT | | YGTLDELQLFTHAVDR | |
| | TCGCCAGAAGTCTTTCGG | | WDIKYLEQLPEYMQIC | |
| | AATTTCATGGATGAAACC | | FLALFNTVNERSYDFL | |
| | GGAAATTTCAAAAAAAA | | LDKGFNVIPHSSYRWA | |
| | CCTGTGCGATGACATAA | | ELCKTYLIEANWYHSG | |
| | AAGGACTCCTTAGCTTGT | | YKPSLNEYLNQGLISV | |
| | ATGAAGCGAGCTATTTGC | | AGPHALSHTYLCMTDS | |
| | TCACGGAAGGTGAAACC | | LKEKHILDLRTNPPVIK | |
| | ATAATGGATTCAGCCCA | | WVSILVRLADDLGTST | |
| | GGCGTTTGCTACCCATCA | | DELKRGDNPKSIQCHM | |
| | CCTTAAACAGAAACTGG | | HDTGCNEEETRAYIKN | |
| | AGGAAAATATGAATAAA | | LIGSTWKKINKDVLMN | |
| | AATCTTGGAGATGAGAT | | FEYSMDFRTAAMNGA | |
| | AGCCCATGCGCTGGAATT | | RVSQFMYQYDDDGHG | |
| | GCCGCTGCACTGGAGAG | | VPEGKSKERVCSLIVEP | |
| | TCCCCAAGCTGGACGTA | | IPLP | |
| | AGATGGTCCATTGACGCT | | | |
| | TATGAGCGTCGACAGGA | | | |
| | TATGAATCCACTTCTTTT | | | |
| | GGAGCTGGCCAAACTGG | | | |
| | ATTTTAATATCGCCCAGA | | | |
| | GTATGTACCAAGATGAA | | | |
| | TTAAAAGAATTAAGTCGT | | | |
| | TGGTATTCAAAAACACA | | | |
| | CCTGCCGGAAAAATTGG | | | |
| | CGTTCGCACGTGATCGCC | | | |
| | TTGTGGAATCCTACTTGT | | | |
| | GGGGACTTGGATTAGCA | | | |
| | TCAGAACCCCATCATAA | | | |
| | ATATTGTCGCATGATGGT | | | |
| | GGCCCAGAGTACTACCCT | | | |
| | GATTAGCATCATCGATGA | | | |
| | TATATATGATGTTTATGG | | | |
| | TACGCTGGATGAATTGCA | | | |
| | GCTGTTTACGCATGCAGT | | | |
| | CGACCGTTGGGACATTA | | | |
| | AATATCTGGAACAATTAC | | | |
| | CCGAATACATGCAGATTT | | | |
| | GTTTCTTAGCGTTGTTTA | | | |
| | ATACTGTGAACGAGCGTT | | | |
| | CATATGACTTTTTACTCG | | | |
| | ACAAAGGTTTTAACGTTA | | | |
| | TCCCGCACTCGAGTTATC | | | |
| | GGTGGGCAGAGCTGTGC | | | |
| | AAGACTTACCTGATAGA | | | |
| | GGCGAATTGGTACCACTC | | | |
| | TGGCTATAAACCCAGCTT | | | |
| | AAATGAATATCTTAACCA | | | |
| | GGGGCTGATCTCAGTCGC | | | |
| | AGGCCCACATGCCTTGTC | | | |
| | ACACACTTATCTGTGCAT | | | |
| | GACTGATAGTCTTAAGG | | | |
| | AAAAACATATACTCGAC | | | |
| | CTGCGCACAAATCCTCCT | | | |
| | GTGATCAAATGGGTTAGT | | | |
| | ATCCTTGTAAGACTGGCA | | | |
| | GACGATCTCGGTACTTCT | | | |
| | ACGGACGAATTGAAACG | | | |
| | TGGGGATAATCCAAAGT | | | |
| | CAATCCAGTGCCATATGC | | | |
| | ATGACACTGGCTGTAATG | | | |
| | AAGAGGAGACACGCGCC | | | |
| | TACATCAAAAATTTAATT | | | |
| | GGTTCCACCTGGAAAAA | | | |
| | GATTAATAAAGATGTTCT | | | |
| | CATGAATTTTGAGTATTC | | | |
| | GATGGATTTTCGGACAGC | | | |
| | GGCGATGAATGGTGCGC | | | |
| | GCGTAAGCCAGTTTATGT | | | |
| | ATCAGTACGATGATGAT | | | |
| | GGACACGGGGTGCCTGA | | | |
| | GGGCAAGTCGAAAGAAC | | | |
| | GTGTTTGTTCCCTGATCG | | | |
| | TCGAACCTATTCCACTGC | | | |
| | CTTAG | | | |
| γ-Humulene | ATGGCTCAAATCAGCGA | 20 | MAQISESVSPSTDLKST | 7 |
| synthase | ATCAGTGTCTCCAAGCAC | | ESSITSNRHGNMWEDD | |
| from Abies | CGACCTTAAAAGCACGG | | RIQSLNSPYGAPAYQE | |
| grandis | AATCTTCTATTACCAGCA | | RSEKLIEEIKLLFLSDM | |
| (O64405) | ACCGCCACGGTAACATG | | DDSCNDSDRDLIKRLEI | |
| | TGGGAAGATGACCGCAT | | VDTVECLGIDRHFQPEI | |
| | TCAGAGCTTAAACAGCC | | KLALDYVYRCWNERG | |
| | CATATGGCGCACCCGCTT | | IGEGSRDSLKKDLNAT | |
| | ATCAGGAACGTAGCGAA | | ALGFRALRLHRYNVSS | |
| | AAATTGATTGAAGAAAT | | GVLENFRDDNGQFFCG | |
| | TAAGCTCCTGTTTCTGTC | | STVEEEGAEAYNKHV | |
| | CGATATGGACGATAGTT | | RCMLSLSRASNILFPGE | |
| | GCAATGATTCGGATCGC | | KVMEEAKAFTTNYLK | |
| | GACTTGATCAAACGCCTG | | KVLAGREATHVDESLL | |
| | GAGATCGTAGATACGGT | | GEVKYALEFPWHCSV | |
| | TGAGTGTCTGGGCATTGA | | QRWEARSFIEIFGQIDS | |
| | TCGTCATTTCCAACCTGA | | ELKSNLSKKMLELAKL | |
| | AATTAAGCTGGCGCTGG | | DFNILQCTHQKELQIIS | |
| | ATTACGTGTACCGTTGCT | | RWFADSSIASLNFYRK | |
| | GGAATGAGCGTGGCATC | | CYVEFYFWMAAAISEP | |
| | GGAGAAGGTAGCCGTGA | | EFSGSRVAFTKIAILMT | |
| | TAGCTTAAAAAAGGACC | | MLDDLYDTHGTLDQL | |
| | TGAATGCGACCGCCTTGG | | KIFTEGVRRWDVSLVE | |
| | GCTTTCGGGCTTTACGCT | | GLPDFMKIAFEFWLKT | |
| | TACACCGTTATAATGTAA | | SNELIAEAVKAQGQD | |
| | GCTCAGGAGTGCTGGAG | | MAAYIRKNAWERYLE | |
| | AACTTCCGTGATGACAAT | | AYLQDAEWIATGHVP | |
| | GGTCAATTCTTTTGCGGT | | TFDEYLNNGTPNTGM | |
| | TCTACTGTGGAGGAGGA | | CVLNLIPLLLMGEHLPI | |
| | AGGCGCGGAGGCCTACA | | DILEQIFLPSRFHHLIEL | |
| | ATAAACATGTACGTTGCA | | ASRLVDDARDFQAEK | |
| | TGCTGTCCCTGTCCCGCG | | DHGDLSCIECYLKDHP | |
| | CTTCCAATATTTTATTCC | | ESTVEDALNHVNGLLG | |
| | CGGGCGAGAAAGTGATG | | NCLLEMNWKFLKKQD | |
| | GAAGAAGCGAAGGCGTT | | SVPLSCKKYSFHVLAR | |
| | TACGACCAACTATCTTAA | | SIQFMYNQGDGFSISN | |
| | GAAAGTCCTGGCGGGTC | | KVIKDQVQKVLIVPVPI | |
| | GTGAAGCAACTCATGTC | | | |
| | GACGAGAGTCTCCTTGG | | | |
| | AGAGGTCAAGTATGCAC | | | |
| | TAGAATTTCCGTGGCATT | | | |
| | GTTCCGTGCAGCGCTGGG | | | |
| | AGGCACGTTCTTTTATCG | | | |
| | AAATTTTCGGTCAGATTG | | | |
| | ATAGTGAACTGAAAAGC | | | |
| | AACCTCTCTAAAAAAAT | | | |
| | GCTCGAACTCGCAAAAC | | | |
| | TTGATTTTAACATACTCC | | | |
| | AGTGTACGCATCAAAAA | | | |
| | GAGCTCCAGATCATTAGT | | | |
| | CGATGGTTCGCCGATTCA | | | |
| | AGTATCGCAAGTCTGAA | | | |
| | CTTTTACCGTAAATGCTA | | | |
| | TGTGGAATTTTACTTCTG | | | |
| | GATGGCCGCGGCAATTTC | | | |
| | AGAACCAGAATTTAGTG | | | |
| | GCTCTCGCGTGGCATTCA | | | |
| | CTAAAATTGCGATCTTGA | | | |
| | TGACAATGTTAGATGACT | | | |
| | TATACGACACGCATGGG | | | |
| | ACGCTGGATCAATTGAA | | | |
| | AATATTTACCGAAGGTGT | | | |
| | GCGCAGGTGGGACGTGT | | | |
| | CGCTGGTGGAGGGCCTG | | | |
| | CCGGATTTCATGAAAATT | | | |
| | GCCTTTGAGTTCTGGTTA | | | |
| | AAGACCTCCAACGAACT | | | |
| | GATTGCGGAGGCGGTTA | | | |
| | AGGCCCAAGGCCAGGAT | | | |
| | ATGGCGGCCTATATCCGC | | | |
| | AAAAACGCTTGGGAACG | | | |
| | CTATCTGGAAGCGTATTT | | | |
| | GCAGGATGCCGAATGGA | | | |
| | TCGCCACCGGTCACGTTC | | | |
| | CGACATTCGATGAATATC | | | |
| | TGAACAATGGCACCCCC | | | |
| | AACACCGGTATGTGTGTA | | | |
| | CTTAATCTGATCCCGTTG | | | |
| | CTGCTTATGGGCGAACAC | | | |
| | TTGCCGATCGATATTCTT | | | |
| | GAACAGATCTTTCTGCCG | | | |
| | AGCCGGTTCCACCATCTG | | | |
| | ATTGAACTGGCTAGCCG | | | |
| | ACTGGTCGATGATGCGA | | | |
| | GAGATTTTCAAGCCGAA | | | |
| | AAAGATCATGGTGATTTA | | | |
| | TCCTGCATCGAATGCTAC | | | |
| | CTGAAAGACCATCCGGA | | | |
| | ATCAACAGTTGAAGACG | | | |
| | CCCTGAATCACGTCAACG | | | |
| | GCCTGCTGGGGAATTGTT | | | |
| | TGCTGGAAATGAATTGG | | | |
| | AAATTTCTGAAAAAACA | | | |
| | GGACTCGGTACCTCTGTC | | | |
| | GTGTAAAAAATACTCATT | | | |
| | CCACGTCCTGGCGCGGTC | | | |
| | GATTCAGTTTATGTATAA | | | |
| | CCAGGGGGACGGGTTTT | | | |
| | CGATTTCGAACAAAGTTA | | | |
| | TTAAAGACCAGGTCCAG | | | |
| | AAAGTTCTAATCGTTCCG | | | |
| | GTTCCTATATAA | | | |
| Sesquiterpene | ATGAACCAGCTGGCAAT | 22 | MNQLAMVNTTITRPLA | 23 |
| synthase | GGTTAATACAACTATCAC | | NYHSSVWGNYFLSYTP | |
| 14b from | CCGCCCATTAGCTAATTA | | QLTEISSQEKRELEELK | |
| Solanum | CCATTCGTCCGTCTGGGG | | EKVRQMLVETPDNST | |
| habrochaites | TAACTATTTCCTCAGTTA | | QKLVLIDTIQRLGVAY | |
| (G8H5N1) | TACTCCTCAGCTGACAGA | | HFENHIKISIQNIFDEFE | |
| | AATTAGTTCACAGGAGA | | KNKNKDNDDDLCVVA | |
| | AGCGTGAACTTGAAGAA | | LRFRLVRGQRHYMSS | |
| | CTGAAGGAAAAAGTTCG | | DVFTRFTNDDGKFKET | |
| | GCAAATGCTGGTAGAAA | | LTKDVQGLLNLYEAT | |
| | CCCCAGATAATTCGACTC | | HLRVHGEEILEEALSFT | |
| | AAAAATTAGTCTTAATCG | | VTHLKSMSPKLDNSLK | |
| | ATACGATTCAACGTCTGG | | AQVSEALFQPIHTNIPR | |
| | GCGTAGCATATCATTTTG | | VVARKYIRIYENIESHD | |
| | AAAACCATATCAAAATA | | DLLLKFAKLDFHILQK | |
| | AGTATTCAGAATATTTTC | | MHQRELSELTRWWKD | |
| | GATGAGTTTGAAAAAAA | | LDHSNKYPYARDKLV | |
| | TAAAAATAAAGATAATG | | ECYFWAIGVYFGPQYK | |
| | ATGATGACTTGTGTGTTG | | RARRTLTKLIVIITITDD | |
| | TCGCTCTTCGTTTTAGAC | | LYDAYATYDELVPYT | |
| | TGGTCCGGGGGCAGCGT | | NAVERCEISAMHSISPY | |
| | CATTACATGTCCAGCGAT | | MRPLYQVFLDYFDEM | |
| | GTCTTTACTCGGTTCACA | | EEELTKDGKAHYVYY | |
| | AATGATGACGGTAAATTT | | AKIETNKWIKSYLKEA | |
| | AAAGAGACTCTGACCAA | | EWLKNDIIPKCEEYKR | |
| | AGACGTTCAGGGCTTGCT | | NATITISNQMNLITCLI | |
| | GAACCTGTATGAAGCGA | | VAGEFISKETFEWMIN | |
| | CCCATCTTCGGGTCCATG | | ESLIAPASSLINRLKDDI | |
| | GCGAGGAAATCCTGGAG | | IGHEHEQQREHGASFIE | |
| | GAAGCGTTGTCCTTTACT | | CYVKEYRASKQEAYV | |
| | GTAACGCACCTGAAGTC | | EARRQITNAWKDINTD | |
| | GATGTCGCCTAAGCTGG | | YLHATQVPTFVLEPAL | |
| | ATAACTCACTCAAAGCTC | | NLSRLVDILQEDDFTD | |
| | AGGTTAGTGAGGCACTTT | | SQNFLKDTITLLFVDSV | |
| | TCCAGCCGATACACACTA | | NSTSCG | |
| | ACATCCCACGGGTAGTC | | | |
| | GCACGTAAATATATTCGT | | | |
| | ATTTACGAGAATATTGAG | | | |
| | AGTCATGATGATTTACTG | | | |
| | CTGAAGTTTGCAAAGCTG | | | |
| | GATTTCCATATTCTTCAA | | | |
| | AAAATGCATCAGCGTGA | | | |
| | GCTGTCCGAACTGACAA | | | |
| | GATGGTGGAAAGATCTG | | | |
| | GATCATTCGAATAAATAT | | | |
| | CCGTATGCACGCGACAA | | | |
| | GCTGGTGGAATGTTATTT | | | |
| | TTGGGCTATTGGCGTATA | | | |
| | CTTTGGCCCCCAGTATAA | | | |
| | GCGTGCGCGTCGAACGC | | | |
| | TGACAAAGCTGATTGTTA | | | |
| | TCATAACCATCACTGATG | | | |
| | ACTTATATGATGCTTACG | | | |
| | CGACGTACGATGAATTG | | | |
| | GTGCCCTATACAAACGC | | | |
| | GGTAGAACGATGTGAAA | | | |
| | TATCGGCGATGCACTCGA | | | |
| | TTTCTCCATATATGCGCC | | | |
| | CCTTGTATCAAGTGTTTC | | | |
| | TGGATTATTTCGATGAAA | | | |
| | TGGAAGAGGAGTTAACT | | | |
| | AAAGATGGCAAAGCGCA | | | |
| | TTATGTGTATTATGCTAA | | | |
| | GATCGAAACGAACAAAT | | | |
| | GGATCAAATCCTATTTGA | | | |
| | AGGAAGCGGAATGGCTG | | | |
| | AAAAATGACATTATCCC | | | |
| | GAAATGTGAAGAATATA | | | |
| | AACGTAATGCTACAATTA | | | |
| | CGATTTCTAATCAGATGA | | | |
| | ACCTGATTACGTGCTTGA | | | |
| | TTGTCGCAGGTGAATTCA | | | |
| | TAAGCAAAGAGACCTTT | | | |
| | GAATGGATGATTAACGA | | | |
| | GAGTCTGATTGCGCCCGC | | | |
| | ATCTAGCCTCATTAACCG | | | |
| | TCTCAAGGATGATATTAT | | | |
| | AGGTCACGAGCATGAGC | | | |
| | AACAGCGTGAGCACGGC | | | |
| | GCAAGTTTTATTGAGTGC | | | |
| | TACGTCAAAGAGTATCGT | | | |
| | GCATCCAAGCAGGAGGC | | | |
| | GTACGTAGAGGCACGCC | | | |
| | GTCAGATCACAAATGCA | | | |
| | TGGAAGGATATAAACAC | | | |
| | AGACTACCTTCATGCGAC | | | |
| | TCAAGTTCCGACCTTCGT | | | |
| | ACTTGAGCCCGCTCTGAA | | | |
| | CCTCAGCCGTTTGGTAGA | | | |
| | TATCCTGCAGGAGGACG | | | |
| | ATTTTACCGATTCTCAGA | | | |
| | ATTTTCTGAAAGATACCA | | | |
| | TTACACTGCTGTTCGTGG | | | |
| | ACAGTGTAAACTCTACAT | | | |
| | CATGCGGCTAA | | | |
| Artemisia | ATGGCCCTGACCGAAGA | 298 | MALTEEKPIRPIANFPP | 4 |
| annua (Sweet | GAAACCGATCCGCCCGA | | SIWGDQFLIYEKQVEQ | |
| wormwood) | TCGCTAACTTCCCGCCGT | | GVEQIVNDLKKEVRQL | |
| Amorpha- | CTATCTGGGGTGACCAGT | | LKEALDIPMKHANLLK | |
| 4,11-diene | TCCTGATCTACGAAAAGC | | LIDEIQRLGIPYHFEREI | |
| synthase | AGGTTGAGCAGGGTGTT | | DHALQCIYETYGDNW | |
| (Q9AR04) | GAACAGATCGTAAACGA | | NGDRSSLWFRLMRKQ | |
| | CCTGAAGAAAGAAGTTC | | GYYVTCDVFNNYKDK | |
| | GTCAGCTGCTGAAAGAA | | NGAFKQSLANDVEGL | |
| | GCTCTGGACATCCCGATG | | LELYEATSMRVPGEIIL | |
| | AAACACGCTAACCTGTTG | | EDALGFTRSRLSIMTK | |
| | AAGCTGATCGACGAGAT | | DAFSTNPALFTEIQRAL | |
| | CCAGCGTCTGGGTATCCC | | KQPLWKRLPRIEAAQY | |
| | GTACCACTTCGAACGCG | | IPFYQQQDSHNKTLLK | |
| | AAATCGACCACGCACTG | | LAKLEFNLLQSLHKEE | |
| | CAGTGCATCTACGAAAC | | LSHVCKWWKAFDIKK | |
| | CTACGGCGACAACTGGA | | NAPCLRDRIVECYFWG | |
| | ACGGCGACCGTTCTTCTC | | LGSGYEPQYSRARVFF | |
| | TGTGGTTTCGTCTGATGC | | TKAVAVITLIDDTYDA | |
| | GTAAACAGGGCTACTAC | | YGTYEELKIFTEAVER | |
| | GTTACCTGTGACGTTTTT | | WSITCLDTLPEYMKPI | |
| | AACAACTACAAGGACAA | | YKLFMDTYTEMEEFL | |
| | GAACGGTGCTTTCAAAC | | AKEGRTDLFNCGKEFV | |
| | AGTCTCTGGCTAACGACG | | KEFVRNLMVEAKWAN | |
| | TTGAAGGCCTGCTGGAA | | EGHIPTTEEHDPVVIIT | |
| | CTGTACGAAGCGACCTCC | | GGANLLTTTCYLGMS | |
| | ATGCGTGTACCGGGTGA | | DIFTKESVEWAVSAPP | |
| | AATCATCCTGGAGGACG | | LFRYSGILGRRLNDLM | |
| | CGCTGGGTTTCACCCGTT | | THKAEQERKHSSSSLE | |
| | CTCGTCTGTCCATTATGA | | SYMKEYNVNEEYAQT | |
| | CTAAAGACGCTTTCTCTA | | LIYKEVEDVWKDINRE | |
| | CTAACCCGGCTCTGTTCA | | YLTTKNIPRPLLMAVIY | |
| | CCGAAATCCAGCGTGCTC | | LCQFLEVQYAGKDNFT | |
| | TGAAACAGCCGCTGTGG | | RMGDEYKHLIKSLLVY | |
| | AAACGTCTGCCGCGTATC | | PMSI* | |
| | GAAGCAGCACAGTACAT | | | |
| | TCCGTTTTACCAGCAGCA | | | |
| | GGACTCTCACAACAAGA | | | |
| | CCCTGCTGAAACTGGCTA | | | |
| | AGCTGGAATTCAACCTGC | | | |
| | TGCAGTCTCTGCACAAAG | | | |
| | AAGAACTGTCTCACGTTT | | | |
| | GTAAGTGGTGGAAGGCA | | | |
| | TTTGACATCAAGAAAAA | | | |
| | CGCGCCGTGCCTGCGTGA | | | |
| | CCGTATCGTTGAATGTTA | | | |
| | CTTCTGGGGTCTGGGTTC | | | |
| | TGGTTATGAACCACAGTA | | | |
| | CTCCCGTGCACGTGTGTT | | | |
| | CTTCACTAAAGCTGTAGC | | | |
| | TGTTATCACCCTGATCGA | | | |
| | TGACACTTACGATGCTTA | | | |
| | CGGCACCTACGAAGAAC | | | |
| | TGAAGATCTTTACTGAAG | | | |
| | CTGTAGAACGCTGGTCTA | | | |
| | TCACTTGCCTGGACACTC | | | |
| | TGCCGGAGTACATGAAA | | | |
| | CCGATCTACAAACTGTTC | | | |
| | ATGGATACCTACACCGA | | | |
| | AATGGAGGAATTCCTGG | | | |
| | CAAAAGAAGGCCGTACC | | | |
| | GACCTGTTCAACTGCGGT | | | |
| | AAAGAGTTTGTTAAAGA | | | |
| | ATTCGTACGTAACCTGAT | | | |
| | GGTTGAAGCTAAATGGG | | | |
| | CTAACGAAGGCCATATC | | | |
| | CCGACTACCGAAGAACA | | | |
| | TGACCCGGTTGTTATCAT | | | |
| | CACCGGCGGTGCAAACC | | | |
| | TGCTGACCACCACTTGCT | | | |
| | ATCTGGGTATGTCCGACA | | | |
| | TCTTTACCAAGGAATCTG | | | |
| | TTGAATGGGCTGTTTCTG | | | |
| | CACCGCCGCTGTTCCGTT | | | |
| | ACTCCGGTATTCTGGGTC | | | |
| | GTCGTCTGAACGACCTGA | | | |
| | TGACCCACAAAGCAGAG | | | |
| | CAGGAACGTAAACACTC | | | |
| | TTCCTCCTCTCTGGAATC | | | |
| | CTACATGAAGGAATATA | | | |
| | ACGTTAACGAGGAGTAC | | | |
| | GCACAGACTCTGATCTAT | | | |
| | AAAGAAGTTGAAGACGT | | | |
| | ATGGAAAGACATCAACC | | | |
| | GTGAATACCTGACTACTA | | | |
| | AAAACATCCCGCGCCCG | | | |
| | CTGCTGATGGCAGTAATC | | | |
| | TACCTGTGCCAGTTCCTG | | | |
| | GAAGTACAGTACGCTGG | | | |
| | TAAAGATAACTTCACTCG | | | |
| | CATGGGCGACGAATACA | | | |
| | AACACCTGATCAAATCCC | | | |
| | TGCTGGTTTACCCGATGT | | | |
| | CCATCTGA | | | |

TABLE 32
Cytochromes 450

| Name | DNA | SEQ ID NO. | AA | SEQ ID NO. |
|---|---|---|---|---|
| BM3 01-BM3 | ATGACAATTAAAGAAATG | 162 | MTIKEMPQPKTFGE | 163 |
| (R47L Y51F | CCTCAGCCAAAAACGTTT | | LKNLPLLNTDKPVQ | |
| A82T F87A | GGAGAGCTTAAAAATTTA | | ALMKIADELGEIFKF | |
| I401P T463P | CCGTTATTAAACACAGAT | | EAPGLVTRFLSSQRL | |
| V702I) | AAACCGGTTCAAGCTTTG | | IKEACDESRFDKNLS | |
| | ATGAAAATTGCGGATGAA | | QALKFVRDFTGDGL | |
| | TTAGGAGAAATCTTTAAA | | ATSWTHEKNWKKA | |
| | TTCGAGGCGCCTGGTCTG | | HNILLPSFSQQAMK | |
| | GTAACGCGCTTCTTATCA | | GYHAMMVDIAVQL | |
| | AGTCAGCGTCTAATTAAA | | VQKWERLNADEHIE | |
| | GAAGCATGCGATGAATCA | | VPEDMTRLTLDTIGL | |
| | CGCTTTGATAAAAACTTA | | CGFNYRFNSFYRDQ | |
| | AGTCAAGCGCTTAAATTT | | PHPFITSMVRALDEA | |
| | GTACGTGATTTTACAGGA | | MNKLQRANPDDPA | |
| | GACGGGTTAGCGACAAGC | | YDENKRQFQEDIKV | |
| | TGGACGCACGAAAAAAAT | | MNDLVDKIIADRKA | |
| | TGGAAAAAAGCGCATAAT | | SGEQSDDLLTHMLN | |
| | ATCTTACTTCCAAGCTTCA | | GKDPETGEPLDDENI | |
| | GTCAGCAGGCAATGAAAG | | RYQIITFLIAGHETTS | |
| | GCTATCATGCGATGATGG | | GLLSFALYFLVKNP | |
| | TCGATATCGCCGTGCAGC | | HVLQKAAEEAARVL | |
| | TTGTTCAAAAGTGGGAGC | | VDPVPSYKQVKQLK | |
| | GTCTAAATGCAGATGAGC | | YVGMVLNEALRLW | |
| | ATATTGAAGTACCCGAAG | | PTAPAFSLYAKEDT | |
| | ATATGACACGTTTAACGC | | VLGGEYPLEKGDEL | |
| | TTGATACAATTGGTCTTTG | | MVLIPQLHRDKTIW | |
| | CGGCTTTAACTATCGCTTT | | GDDVEEFRPERFENP | |
| | AACAGCTTTTACCGAGAT | | SAIPQHAFKPFGNGQ | |
| | CAGCCTCATCCATTTATTA | | RACPGQQFALHEAT | |
| | CAAGTATGGTCCGTGCAC | | LVLGMVLKHFDFED | |
| | TGGATGAAGCAATGAACA | | HTNYELDIKETLTLK | |
| | AGCTGCAGCGAGCAAATC | | PEGFVVKAKSKKIPL | |
| | CAGACGACCCAGCTTATG | | GGIPSPSPEQSAKKV | |
| | ATGAAAACAAGCGCCAGT | | RKKAENAHNTPLLV | |
| | TTCAAGAAGATATCAAGG | | LYGSNMGTAEGTAR | |
| | TGATGAACGACCTAGTAG | | DLADIAMSKGFAPQ | |
| | ATAAAATTATTGCAGATC | | VATLDSHAGNLPRE | |
| | GCAAAGCAAGCGGTGAAC | | GAVLIVTASYNGHP | |
| | AAAGCGATGATTTATTAA | | PDNAKQFVDWLDQ | |
| | CGCATATGCTAAACGGAA | | ASADEVKGVRYSVF | |
| | AAGATCCAGAAACAGGTG | | GCGDKNWATTYQK | |
| | AGCCGCTTGATGACGAGA | | VPAFIDETLAAKGAE | |
| | ACATTCGCTATCAAATTA | | NIADRGEADASDDF | |
| | TTACATTCTTAATTGCGGG | | EGTYEEWREHMWS | |
| | ACACGAAACAACAAGCG | | DVAAYFNLDIENSE | |
| | GTCTTTTATCATTTGCGCT | | DNKSTLSLQFVDSA | |
| | GTATTTCTTAGTGAAAAA | | ADMPLAKMHGAFS | |
| | TCCACATGTATTACAAAA | | TNVVASKELQQPGS | |
| | AGCAGCAGAAGAAGCAG | | ARSTRHLEIELPKEA | |
| | CACGAGTTCTAGTAGATC | | SYQEGDHLGIIPRNY | |
| | CTGTTCCAAGCTACAAAC | | EGIVNRVTARFGLD | |
| | AAGTCAAACAGCTTAAAT | | ASQQIRLEAEEEKLA | |
| | ATGTCGGCATGGTCTTAA | | HLPLAKTVSVEELL | |
| | ACGAAGCGCTGCGCTTAT | | QYVELQDPVTRTQL | |
| | GGCCAACTGCTCCTGCGT | | RAMAAKTVCPPHK | |
| | TTTCCCTATATGCAAAAG | | VELEALLEKQAYKE | |
| | AAGATACGGTGCTTGGAG | | QVLAKRLTMLELLE | |
| | GAGAATATCCTTTAGAAA | | KYPACEMEFSEFIAL | |
| | AAGGCGACGAACTAATGG | | LPSIRPRYYSISSSPR | |
| | TTCTGATTCCTCAGCTTCA | | VDEKQASITVSVVS | |
| | CCGTGATAAAACAATTTG | | GEAWSGYGEYKGIA | |
| | GGGAGACGATGTGGAAG | | SNYLAELQEGDTITC | |
| | AGTTCCGTCCAGAGCGTT | | FISTPQSEFTLPKDPE | |
| | TTGAAAATCCAAGTGCGA | | TPLIMVGPGTGVAPF | |
| | TTCCGCAGCATGCGTTTA | | RGFVQARKQLKEKG | |
| | AACCGTTTGGAAACGGTC | | QSLGEAHLYFGCRS | |
| | AGCGTGCGTGTCCAGGTC | | PHEDYLYQEELENA | |
| | AGCAGTTCGCTCTTCATG | | QNEGIITLHTAFSRV | |
| | AAGCAACGCTGGTACTTG | | PNQPKTYVQHVME | |
| | GTATGGTGCTAAAACACT | | QDGKKLIELLDQGA | |
| | TTGACTTTGAAGATCATA | | HFYICGDGSQMAPD | |
| | CAAACTACGAGCTGGATA | | VEATLMKSYAGVH | |
| | TTAAAGAAACTTTAACGT | | QVSEADARLWLQQL | |
| | TAAAACCTGAAGGCTTTG | | EEKGRYAKDVWAG | |
| | TGGTAAAAGCAAAATCGA | | | |
| | AAAAAATTCCGCTTGGCG | | | |
| | GTATTCCTTCACCTAGCCC | | | |
| | TGAACAGTCTGCTAAAAA | | | |
| | AGTACGCAAAAAGGCAG | | | |
| | AAAACGCTCATAATACGC | | | |
| | CGCTGCTTGTGCTATACG | | | |
| | GTTCAAATATGGGAACAG | | | |
| | CTGAAGGAACGGCGCGTG | | | |
| | ATTTAGCAGATATTGCGA | | | |
| | TGAGCAAAGGATTCGCAC | | | |
| | CGCAGGTCGCTACCCTTG | | | |
| | ATTCACACGCCGGAAATC | | | |
| | TTCCGCGCGAAGGAGCTG | | | |
| | TATTAATTGTAACGGCGT | | | |
| | CTTATAACGGACATCCGC | | | |
| | CTGATAACGCAAAGCAAT | | | |
| | TTGTCGACTGGTTAGACC | | | |
| | AAGCGTCTGCTGATGAAG | | | |
| | TAAAAGGCGTTCGCTACT | | | |
| | CCGTATTTGGATGCGGCG | | | |
| | ATAAAAACTGGGCTACTA | | | |
| | CGTATCAAAAAGTGCCTG | | | |
| | CTTTTATCGATGAAACGC | | | |
| | TTGCCGCTAAAGGGGCAG | | | |
| | AAAACATCGCTGACCGCG | | | |
| | GTGAAGCAGATGCAAGCG | | | |
| | ACGACTTTGAAGGCACAT | | | |
| | ATGAAGAATGGCGTGAAC | | | |
| | ATATGTGGAGTGACGTAG | | | |
| | CAGCCTACTTTAACCTCG | | | |
| | ACATTGAAAACAGTGAAG | | | |
| | ATAATAAATCTACTCTTTC | | | |
| | ACTTCAATTTGTCGACAG | | | |
| | CGCCGCGGATATGCCGCT | | | |
| | TGCGAAAATGCATGGTGC | | | |
| | GTTTTCAACGAACGTCGT | | | |
| | AGCAAGCAAAGAACTTCA | | | |
| | ACAGCCAGGCAGTGCACG | | | |
| | AAGCACGCGACACCTTGA | | | |
| | AATTGAACTTCCAAAAGA | | | |
| | AGCTTCTTATCAAGAAGG | | | |
| | AGATCATTTAGGTATTATT | | | |
| | CCTCGCAACTATGAAGGA | | | |
| | ATAGTAAACCGTGTAACA | | | |
| | GCAAGGTTCGGACTAGAT | | | |
| | GCATCACAGCAAATCCGT | | | |
| | CTGGAAGCAGAAGAAGA | | | |
| | AAAATTAGCTCATTTGCC | | | |
| | ACTCGCTAAAACAGTATC | | | |
| | CGTAGAAGAGCTTCTGCA | | | |
| | ATACGTGGAGCTTCAAGA | | | |
| | TCCTGTTACGCGCACGCA | | | |
| | GCTTCGCGCAATGGCTGC | | | |
| | TAAAACGGTCTGCCCGCC | | | |
| | GCATAAAGTAGAGCTTGA | | | |
| | AGCCTTGCTTGAAAAGCA | | | |
| | AGCCTACAAAGAACAAGT | | | |
| | GCTGGCAAAACGTTTAAC | | | |
| | AATGCTTGAACTGCTTGA | | | |
| | AAAATACCCGGCGTGTGA | | | |
| | AATGGAATTCAGCGAATT | | | |
| | TATCGCCCTTCTGCCAAG | | | |
| | CATACGCCCGCGTTATTA | | | |
| | CTCGATTTCTTCATCACCT | | | |
| | CGTGTCGATGAAAAACAA | | | |
| | GCAAGCATCACGGTCAGC | | | |
| | GTTGTCTCAGGAGAAGCG | | | |
| | TGGAGCGGATATGGAGAA | | | |
| | TATAAAGGAATTGCGTCG | | | |
| | AACTATCTTGCTGAGCTG | | | |
| | CAAGAAGGAGATACGATT | | | |
| | ACGTGCTTTATTTCCACAC | | | |
| | CGCAGTCAGAATTTACGC | | | |
| | TGCCAAAAGACCCTGAAA | | | |
| | CGCCGCTTATCATGGTCG | | | |
| | GACCGGGAACAGGCGTCG | | | |
| | CGCCGTTTAGAGGCTTCG | | | |
| | TGCAGGCTCGCAAGCAGC | | | |
| | TAAAAGAAAAAGGACAG | | | |
| | TCGCTTGGAGAAGCGCAT | | | |
| | TTATACTTCGGCTGCCGTT | | | |
| | CACCTCATGAAGACTATC | | | |
| | TGTATCAAGAAGAGCTTG | | | |
| | AAAACGCCCAAAATGAAG | | | |
| | GCATCATTACGCTTCATA | | | |
| | CCGCTTTTTCTCGCGTGCC | | | |
| | AAATCAGCCAAAAACATA | | | |
| | CGTTCAGCACGTGATGGA | | | |
| | ACAAGACGGCAAGAAATT | | | |
| | GATTGAACTTCTTGATCA | | | |
| | AGGAGCGCACTTCTATAT | | | |
| | TTGCGGAGACGGAAGCCA | | | |
| | AATGGCACCTGACGTTGA | | | |
| | AGCAACGCTTATGAAAAG | | | |
| | CTATGCTGGCGTTCACCA | | | |
| | AGTGAGTGAAGCAGACGC | | | |
| | TCGCTTATGGCTGCAGCA | | | |
| | GCTAGAGGAAAAAGGCC | | | |
| | GATACGCAAAAGACGTGT | | | |
| | GGGCTGGGTAA | | | |
| BM3 02-BM3 | ATGGCAATTAAAGAAATG | 164 | MAIKEMPQPKTFGE | 165 |
| (R47L Y51F | CCTCAGCCAAAAACGTTT | | LKNLPLLNTDKPVQ | |
| F87A A328L) | GGAGAGCTTAAAAATTTA | | ALMKIADELGEIFKF | |
| | CCGTTATTAAACACAGAT | | EAPGLVTRFLSSQRL | |
| | AAACCGGTTCAAGCTTTG | | IKEACDESRFDKNLS | |
| | ATGAAAATTGCGGATGAA | | QALKFVRDFAGDGL | |
| | TTAGGAGAAATCTTTAAA | | ATSWTHEKNWKKA | |
| | TTCGAGGCGCCTGGTCTG | | HNILLPSFSQQAMK | |
| | GTAACGCGCTTTTTATCA | | GYHAMMVDIAVQL | |
| | AGTCAGCGTCTAATTAAA | | VQKWERLNADEHIE | |
| | GAAGCATGCGATGAATCA | | VPEDMTRLTLDTIGL | |
| | CGCTTTGATAAAAACTTA | | CGFNYRFNSFYRDQ | |
| | AGTCAAGCGCTTAAATTT | | PHPFITSMVRALDEA | |
| | GTACGTGATTTTGCAGGA | | MNKLQRANPDDPA | |
| | GACGGGTTAGCGACAAGC | | YDENKRQFQEDIKV | |
| | TGGACGCATGAAAAAAAT | | MNDLVDKIIADRKA | |
| | TGGAAAAAAGCGCATAAT | | SGEQSDDLLTHMLN | |
| | ATCTTACTTCCAAGCTTCA | | GKDPETGEPLDDENI | |
| | GTCAGCAGGCAATGAAAG | | RYQIITFLIAGHETTS | |
| | GCTATCATGCGATGATGG | | GLLSFALYFLVKNP | |
| | TCGATATCGCCGTGCAGC | | HVLQKAAEEAARVL | |
| | TTGTTCAAAAGTGGGAGC | | VDPVPSYKQVKQLK | |
| | GTCTAAATGCAGATGAGC | | YVGMVLNEALRLW | |
| | ATATTGAAGTACCGGAAG | | PTLPAFSLYAKEDTV | |
| | ACATGACACGTTTAACGC | | LGGEYPLEKGDELM | |
| | TTGATACAATTGGTCTTTG | | VLIPQLHRDKTIWG | |
| | CGGCTTTAACTATCGCTTT | | DDVEEFRPERFENPS | |
| | AACAGCTTTTACCGAGAT | | AIPQHAFKPFGNGQ | |
| | CAGCCTCATCCATTTATTA | | RACIGQQFALHEAT | |
| | CAAGTATGGTCCGTGCAC | | LVLGMMLKHFDFED | |
| | TGGATGAAGCAATGAACA | | HTNYELDIKETLTLK | |
| | AGCTGCAGCGAGCAAATC | | PEGFVVKAKSKKIPL | |
| | CAGACGACCCAGCTTATG | | GGIPSPSTEQSAKKV | |
| | ATGAAAACAAGCGCCAGT | | RKKAENAHNTPLLV | |
| | TTCAAGAAGATATCAAGG | | LYGSNMGTAEGTAR | |
| | TGATGAACGACCTAGTAG | | DLADIAMSKGFAPQ | |
| | ATAAAATTATTGCAGATC | | VATLDSHAGNLPRE | |
| | GCAAAGCAAGCGGTGAAC | | GAVLIVTASYNGHP | |
| | AAAGCGATGATTTATTAA | | PDNAKQFVDWLDQ | |
| | CGCATATGCTAAACGGAA | | ASADEVKGVRYSVF | |
| | AAGATCCAGAAACGGGTG | | GCGDKNWATTYQK | |
| | AGCCGCTTGATGACGAGA | | VPAFIDETLAAKGAE | |
| | ACATTCGCTATCAAATTA | | NIADRGEADASDDF | |
| | TTACATTCTTAATTGCGGG | | EGTYEEWREHMWS | |
| | ACACGAAACAACAAGTGG | | DVAAYFNLDIENSE | |
| | TCTTTTATCATTTGCGCTG | | DNKSTLSLQFVDSA | |
| | TATTTCTTAGTGAAAAAT | | ADMPLAKMHGAFS | |
| | CCACATGTATTACAAAAA | | TNVVASKELQQPGS | |
| | GCAGCAGAAGAAGCAGC | | ARSTRHLEIELPKEA | |
| | ACGAGTTCTAGTAGATCC | | SYQEGDHLGVIPRN | |
| | TGTTCCAAGCTACAAACA | | YEGIVNRVTARFGL | |
| | AGTCAAACAGCTTAAATA | | DASQQIRLEAEEEKL | |
| | TGTCGGCATGGTCTTAAA | | AHLPLAKTVSVEEL | |
| | CGAAGCGCTGCGCTTATG | | LQYVELQDPVTRTQ | |
| | GCCAACTCTGCCTGCGTTT | | LRAMAAKTVCPPHK | |
| | TCCCTATATGCAAAAGAA | | VELEALLEKQAYKE | |
| | GATACGGTGCTTGGAGGA | | QVLAKRLTMLELLE | |
| | GAATATCCTTTAGAAAAA | | KYPACEMKFSEFIAL | |
| | GGCGACGAACTAATGGTT | | LPSIRPRYYSISSSPR | |
| | CTGATTCCTCAGCTTCACC | | VDEKQASITVSVVS | |
| | GTGATAAAACAATTTGGG | | GEAWSGYGEYKGIA | |
| | GAGACGATGTGGAAGAGT | | SNYLAELQEGDTITC | |
| | TCCGTCCAGAGCGTTTTG | | FISTPQSEFTLPKDPE | |
| | AAAATCCAAGTGCGATTC | | TPLIMVGPGTGVAPF | |
| | CGCAGCATGCGTTTAAAC | | RGFVQARKQLKEQG | |
| | CGTTTGGAAACGGTCAGC | | QSLGEAHLYFGCRS | |
| | GTGCGTGTATCGGTCAGC | | PHEDYLYQEELENA | |
| | AGTTCGCTCTTCATGAAG | | QSEGIITLHTAFSRM | |
| | CAACGCTGGTACTTGGTA | | PNQPKTYVQHVME | |
| | TGATGCTAAAACACTTTG | | QDGKKLIELLDQGA | |
| | ACTTTGAAGATCATACAA | | HFYICGDGSQMAPA | |
| | ACTACGAGCTGGATATTA | | VEATLMKSYADVH | |
| | AAGAAACTTTAACGTTAA | | QVSEADARLWLQQL | |
| | AACCTGAAGGCTTTGTGG | | EEKGRYAKDVWAG | |
| | TAAAAGCAAAATCGAAAA | | | |
| | AAATTCCGCTTGGCGGTA | | | |
| | TTCCTTCACCTAGCACTGA | | | |
| | ACAGTCTGCTAAAAAAGT | | | |
| | ACGCAAAAAGGCAGAAA | | | |
| | ACGCTCATAATACGCCGC | | | |
| | TGCTTGTGCTATACGGTTC | | | |
| | AAATATGGGAACAGCTGA | | | |
| | AGGAACGGCGCGTGATTT | | | |
| | AGCAGATATTGCAATGAG | | | |
| | CAAAGGATTTGCACCGCA | | | |
| | GGTCGCAACGCTTGATTC | | | |
| | ACACGCCGGAAATCTTCC | | | |
| | GCGCGAAGGAGCTGTATT | | | |
| | AATTGTAACGGCGTCTTA | | | |
| | TAACGGTCATCCGCCTGA | | | |
| | TAACGCAAAGCAATTTGT | | | |
| | CGACTGGTTAGACCAAGC | | | |
| | GTCTGCTGATGAAGTAAA | | | |
| | AGGCGTTCGCTACTCCGT | | | |
| | ATTTGGATGCGGCGATAA | | | |
| | AAACTGGGCTACTACGTA | | | |
| | TCAAAAAGTGCCTGCTTT | | | |
| | TATCGATGAAACGCTTGC | | | |
| | CGCTAAAGGGGCAGAAA | | | |
| | ACATCGCTGACCGCGGTG | | | |
| | AAGCAGATGCAAGCGACG | | | |
| | ACTTTGAAGGCACATATG | | | |
| | AAGAATGGCGTGAACATA | | | |
| | TGTGGAGTGACGTAGCAG | | | |
| | CCTACTTTAACCTCGACAT | | | |
| | TGAAAACAGTGAAGATAA | | | |
| | TAAATCTACTCTTTCACTT | | | |
| | CAATTTGTCGACAGCGCC | | | |
| | GCGGATATGCCGCTTGCG | | | |
| | AAAATGCACGGTGCGTTT | | | |
| | TCAACGAACGTCGTAGCA | | | |
| | AGCAAAGAACTTCAACAG | | | |
| | CCAGGCAGTGCACGAAGC | | | |
| | ACGCGACATCTTGAAATT | | | |
| | GAACTTCCAAAAGAAGCT | | | |
| | TCTTATCAAGAAGGAGAT | | | |
| | CATTTAGGTGTTATTCCTC | | | |
| | GCAACTATGAAGGAATAG | | | |
| | TAAACCGTGTAACAGCAA | | | |
| | GGTTCGGCCTAGATGCAT | | | |
| | CACAGCAAATCCGTCTGG | | | |
| | AAGCAGAAGAAGAAAAA | | | |
| | TTAGCTCATTTGCCACTCG | | | |
| | CTAAAACAGTATCCGTAG | | | |
| | AAGAGCTTCTGCAATACG | | | |
| | TGGAGCTTCAAGATCCTG | | | |
| | TTACGCGCACGCAGCTTC | | | |
| | GCGCAATGGCTGCTAAAA | | | |
| | CGGTCTGCCCGCCGCATA | | | |
| | AAGTAGAGCTTGAAGCCT | | | |
| | TGCTTGAAAAGCAAGCCT | | | |
| | ACAAAGAACAAGTGCTGG | | | |
| | CAAAACGTTTAACAATGC | | | |
| | TTGAACTGCTTGAAAAAT | | | |
| | ACCCGGCGTGTGAAATGA | | | |
| | AATTCAGCGAATTTATCG | | | |
| | CCCTTCTGCCAAGCATAC | | | |
| | GCCCGCGCTATTACTCGA | | | |
| | TTTCTTCATCACCTCGTGT | | | |
| | CGATGAAAAACAAGCAA | | | |
| | GCATCACGGTCAGCGTTG | | | |
| | TCTCAGGAGAAGCGTGGA | | | |
| | GCGGATATGGAGAATATA | | | |
| | AAGGAATTGCGTCGAACT | | | |
| | ATCTTGCCGAGCTGCAAG | | | |
| | AAGGAGATACGATTACGT | | | |
| | GCTTTATTTCCACACCGCA | | | |
| | GTCAGAATTTACGCTGCC | | | |
| | AAAAGACCCTGAAACGCC | | | |
| | GCTTATCATGGTCGGACC | | | |
| | GGGAACAGGCGTCGCGCC | | | |
| | GTTTAGAGGCTTTGTGCA | | | |
| | GGCGCGCAAACAGCTAAA | | | |
| | AGAACAAGGACAGTCACT | | | |
| | TGGAGAAGCACATTTATA | | | |
| | CTTCGGCTGCCGTTCACCT | | | |
| | CATGAAGACTATCTGTAT | | | |
| | CAAGAAGAGCTTGAAAAC | | | |
| | GCCCAAAGCGAAGGCATC | | | |
| | ATTACGCTTCATACCGCTT | | | |
| | TTTCTCGCATGCCAAATC | | | |
| | AGCCGAAAACATACGTTC | | | |
| | AGCACGTAATGGAACAAG | | | |
| | ACGGCAAGAAATTGATTG | | | |
| | AACTTCTTGATCAAGGAG | | | |
| | CGCACTTCTATATTTGCGG | | | |
| | AGACGGAAGCCAAATGGC | | | |
| | ACCTGCCGTTGAAGCAAC | | | |
| | GCTTATGAAAAGCTATGC | | | |
| | TGACGTTCACCAAGTGAG | | | |
| | TGAAGCAGACGCTCGCTT | | | |
| | ATGGCTGCAGCAGCTAGA | | | |
| | AGAAAAAGGCCGATACGC | | | |
| | AAAAGACGTGTGGGCTGG | | | |
| | GTAA | | | |
| CYP2A6 | GTTGCCTTGTTGGTCTGTC | 166 | VALLVCLTVMVLMS | 167 |
| | TGACTGTCATGGTGTTAA | | VWQQRKSKGKLPPG | |
| | TGAGTGTGTGGCAACAAC | | PTPLPFIGNYLQLNT | |
| | GGAAGAGCAAAGGCAAG | | EQMYNSLMKISERY | |
| | TTACCGCCCGGCCCAACT | | GPVFTIHLGPRRVVV | |
| | CCGCTGCCCTTTATAGGC | | LCGHDAVREALVDQ | |
| | AACTATCTTCAGTTGAAC | | AEEFSGRGEQATFD | |
| | ACAGAGCAGATGTATAAC | | WVFKGYGVVFSNG | |
| | TCCTTGATGAAGATCTCG | | ERAKQLRRFSIATLR | |
| | GAACGTTACGGTCCAGTC | | DFGVGKRGIEERIQE | |
| | TTTACGATACATTTGGGC | | EAGFLIDALRGTGG | |
| | CCTCGGCGGGTTGTTGTA | | ANIDPTFFLSRTVSN | |
| | TTGTGTGGACATGACGCT | | VISSIVFGDRFDYKD | |
| | GTGCGCGAGGCACTTGTA | | KEFLSLLRMMLGIFQ | |
| | GACCAAGCGGAAGAATTC | | FTSTSTGQLYEMFSS | |
| | AGTGGGCGCGGTGAACAA | | VMKHLPGPQQQAFQ | |
| | GCGACATTCGACTGGGTC | | LLQGLEDFIAKKVE | |
| | TTCAAGGGATATGGAGTT | | HNQRTLDPNSPRDFI | |
| | GTTTTCTCTAATGGAGAA | | DSFLIRMQEEEKNPN | |
| | CGGGCAAAACAGCTTCGT | | TEFYLKNLVMTTLN | |
| | CGGTTTAGTATAGCGACA | | LFIGGTETVSTTLRY | |
| | CTTCGGGATTTCGGAGTG | | GFLLLMKHPEVEAK | |
| | GGGAAAAGAGGCATTGA | | VHEEIDRVIGKNRQP | |
| | AGAACGCATTCAAGAGGA | | KFEDRAKMPYMEA | |
| | AGCGGGATTTCTGATAGA | | VIHEIQRFGDVIPMS | |
| | CGCTCTTAGAGGGACAGG | | LARRVKKDTKFRDF | |
| | CGGTGCAAATATCGACCC | | FLPKGTEVYPMLGS | |
| | CACGTTTTTTTTGAGCCGT | | VLRDPSFFSNPQDFN | |
| | ACTGTTAGCAATGTCATT | | PQHFLNEKGQFKKS | |
| | AGCAGCATCGTGTTTGGT | | DAFVPFSIGKRNCFG | |
| | GATCGGTTCGATTACAAG | | EGLARMELFLFFTTV | |
| | GACAAAGAATTTCTGTCG | | MQNFRLKSSQSPKDI | |
| | TTGTTGAGAATGATGTTA | | DVSPKHVGFATIPRN | |
| | GGGATCTTCCAATTTACTT | | YTMSFLPR | |
| | CGACGTCGACTGGGCAGT | | | |
| | TGTACGAGATGTTTTCGTC | | | |
| | GGTAATGAAACATTTGCC | | | |
| | GGGTCCCCAGCAGCAAGC | | | |
| | ATTCCAGCTGTTACAAGG | | | |
| | ATTAGAAGATTTTATAGC | | | |
| | TAAGAAAGTAGAGCATAA | | | |
| | TCAACGGACGTTAGACCC | | | |
| | AAACTCACCAAGAGATTT | | | |
| | CATAGACAGCTTCTTGAT | | | |
| | ACGGATGCAGGAGGAGG | | | |
| | AGAAAAACCCAAATACAG | | | |
| | AATTTTATCTTAAGAATCT | | | |
| | GGTTATGACTACACTTAA | | | |
| | TTTGTTTATAGGGGGTAC | | | |
| | AGAGACAGTGTCGACCAC | | | |
| | GTTGCGTTACGGTTTCCTG | | | |
| | CTGCTGATGAAACACCCC | | | |
| | GAAGTTGAAGCTAAAGTC | | | |
| | CATGAAGAGATAGACCGG | | | |
| | GTTATCGGAAAAAACAGA | | | |
| | CAACCTAAATTCGAGGAT | | | |
| | CGCGCAAAGATGCCTTAC | | | |
| | ATGGAAGCTGTAATACAT | | | |
| | GAAATACAAAGATTCGGT | | | |
| | GATGTTATCCCGATGTCTT | | | |
| | TAGCGCGCCGTGTCAAAA | | | |
| | AGGATACCAAGTTCCGCG | | | |
| | ACTTCTTCCTGCCAAAAG | | | |
| | GAACAGAGGTGTATCCCA | | | |
| | TGTTGGGGTCTGTCTTGA | | | |
| | GAGATCCGTCATTTTTCA | | | |
| | GCAATCCCCAAGATTTTA | | | |
| | ACCCGCAGCATTTTTTGA | | | |
| | ATGAGAAAGGGCAATTCA | | | |
| | AAAAGTCAGACGCTTTCG | | | |
| | TGCCTTTCTCAATAGGCA | | | |
| | AACGGAACTGCTTTGGAG | | | |
| | AGGGGCTGGCCCGGATGG | | | |
| | AACTTTTCTTGTTCTTCAC | | | |
| | AACGGTCATGCAAAATTT | | | |
| | TCGCCTGAAATCATCCCA | | | |
| | ATCACCTAAGGATATCGA | | | |
| | TGTCTCGCCCAAACACGT | | | |
| | CGGGTTTGCCACCATCCC | | | |
| | CCGGAACTACACCATGTC | | | |
| | GTTTCTTCCTCGG | | | |
| CamA P450 | ATGGGAACAACACGCATG | 168 | MGTTRMDTFNPQES | 169 |
| Novosphingobium | GATACCTTCAATCCCCAA | | RLATNFDEAVRAKV | |
| aromaticivorans | GAAAGCCGTCTTGCAACG | | ERPANVPEDRVYEID | |
| DSM 12444 | AACTTTGATGAAGCAGTG | | MYALNGIEDGYHEA | |
| (CYP101D2) | CGTGCCAAGGTCGAGCGC | | WKKVQHPGIPDLIW | |
| | CCCGCTAATGTACCTGAG | | TPFTGGHWIATNGD | |
| | GACCGCGTATATGAAATT | | TVKEVYSDPTRESSE | |
| | GACATGTATGCACTGAAC | | VIFLPKEAGEKYQM | |
| | GGGATTGAAGATGGATAT | | VPTKMDPPEHTPYR | |
| | CACGAAGCGTGGAAAAA | | KALDKGLNLAKIRK | |
| | GGTGCAACATCCAGGCAT | | VEDKVREVASSLIDS | |
| | CCCCGATCTTATTTGGAC | | FAARGECDFAAEYA | |
| | GCCATTTACAGGGGGGCA | | ELFPVHVFMALADL | |
| | CTGGATTGCGACGAATGG | | PLEDIPVLSEYARQM | |
| | TGATACGGTTAAAGAAGT | | TRPEGNTPEEMATD | |
| | GTATAGTGATCCGACCCG | | LEAGNNGFYAYVDP | |
| | CTTCAGCTCCGAAGTCAT | | IIRARVGGDGDDLIT | |
| | CTTTTTACCCAAGGAGGC | | LMVNSEINGERIAHD | |
| | CGGTGAAAAATACCAGAT | | KAQGLISLLLLGGLD | |
| | GGTCCCCACCAAGATGGA | | TVVNFLSFFMIHLAR | |
| | CCCTCCTGAACACACACC | | HPELVAELRSDPLKL | |
| | ATATCGCAAGGCCCTGGA | | MRGAEEMFRRFPVV | |
| | CAAGGGATTGAACCTTGC | | SEARMVAKDQEYK | |
| | CAAGATTCGCAAGGTTGA | | GVFLKRGDMILLPT | |
| | GGACAAGGTCCGCGAAGT | | ALHGLDDAANPEPW | |
| | TGCCTCTAGTCTGATCGAT | | KLDFSRRSISHSTFG | |
| | TCATTCGCCGCCCGCGGA | | GGPHRCAGMHLAR | |
| | GAGTGTGACTTCGCTGCC | | MEVIVTLEEWLKRIP | |
| | GAATATGCTGAGTTATTT | | EFSFKEGETPIYHSGI | |
| | CCTGTTCATGTCTTTATGG | | VAAVENVPLVWPIA | |
| | CGCTGGCTGACCTGCCTC | | R | |
| | TGGAGGACATCCCGGTTC | | | |
| | TTAGCGAATACGCCCGTC | | | |
| | AAATGACCCGCCCTGAAG | | | |
| | GTAATACGCCAGAGGAAA | | | |
| | TGGCTACGGATTTAGAGG | | | |
| | CAGGCAATAATGGTTTTT | | | |
| | ATGCATATGTCGACCCTA | | | |
| | TCATCCGCGCCCGTGTGG | | | |
| | GGGGAGACGGAGATGATC | | | |
| | TTATCACCTTGATGGTTAA | | | |
| | TAGTGAGATTAACGGTGA | | | |
| | GCGCATCGCGCATGACAA | | | |
| | AGCTCAAGGCCTTATCTC | | | |
| | GTTGCTGTTATTGGGAGG | | | |
| | CCTGGATACGGTCGTCAA | | | |
| | TTTCCTGTCCTTCTTTATG | | | |
| | ATTCACCTTGCACGCCAT | | | |
| | CCCGAGCTGGTCGCGGAA | | | |
| | CTTCGTTCGGACCCACTG | | | |
| | AAACTTATGCGCGGCGCC | | | |
| | GAAGAGATGTTTCGCCGT | | | |
| | TTTCCGGTAGTCAGTGAA | | | |
| | GCCCGTATGGTGGCAAAG | | | |
| | GACCAGGAGTATAAGGGG | | | |
| | GTCTTTTTGAAGCGTGGC | | | |
| | GATATGATTTTATTACCTA | | | |
| | CCGCTTTACACGGTCTGG | | | |
| | ACGATGCCGCTAACCCAG | | | |
| | AACCGTGGAAATTAGACT | | | |
| | TTTCACGCCGCTCAATTA | | | |
| | GCCATTCAACTTTTGGAG | | | |
| | GGGGGCCACATCGCTGTG | | | |
| | CAGGTATGCACTTAGCCC | | | |
| | GTATGGAGGTAATCGTTA | | | |
| | CACTGGAGGAGTGGCTTA | | | |
| | AACGTATTCCCGAATTTTC | | | |
| | TTTCAAAGAGGGGGAAAC | | | |
| | CCCAATCTATCACTCTGG | | | |
| | AATCGTAGCAGCTGTCGA | | | |
| | AAACGTCCCCTTGGTGTG | | | |
| | GCCGATCGCACGT | | | |

### Example 16: Amplification of Reporter Protein Expression Using Inverted B2H System

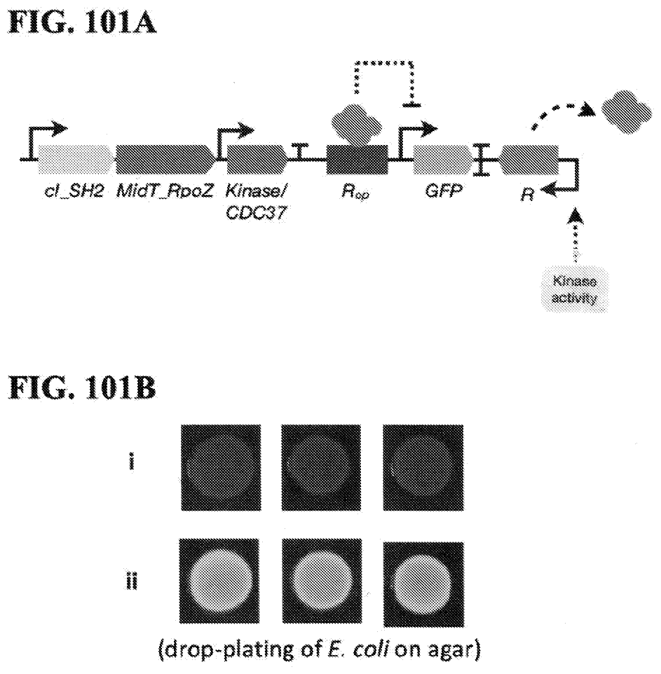

An inverted bacterial two-hybrid system was developed based on the phosphorylation dependent B2H system. DH10BΔRpoω cells (e.g., DH10B cells with the gene for the omega subunit of RNA polymerase knocked out) were produced harboring either (i) the system depicted in FIG. 101A (“inverted B2H”), (ii) system depicted in FIG. 101A with the tyrosine residue of the MidT substrate mutated to a phenylalanine (“inverted B2Hx”), or (iii) a system lacking GFP. These cells were plated on solid media and imaged with a Chemidoc imaging system. A composite image showed that the inverted B2Hx is much more fluorescent than inverted B2H or no GFP cells, demonstrating phosphorylation-dependent transcriptional repression of the GFP. FIG. 101B depicts biological triplicate data of DH10BΔRpoω cells with plasmid-borne versions of B2H systems from FIG. 101A, where cells are seeded on agar plates from drops of liquid culture. Cells harboring inverted B2Hx (ii, bottom row) are much more fluorescent than cells harboring inverted B2H (i, top row).

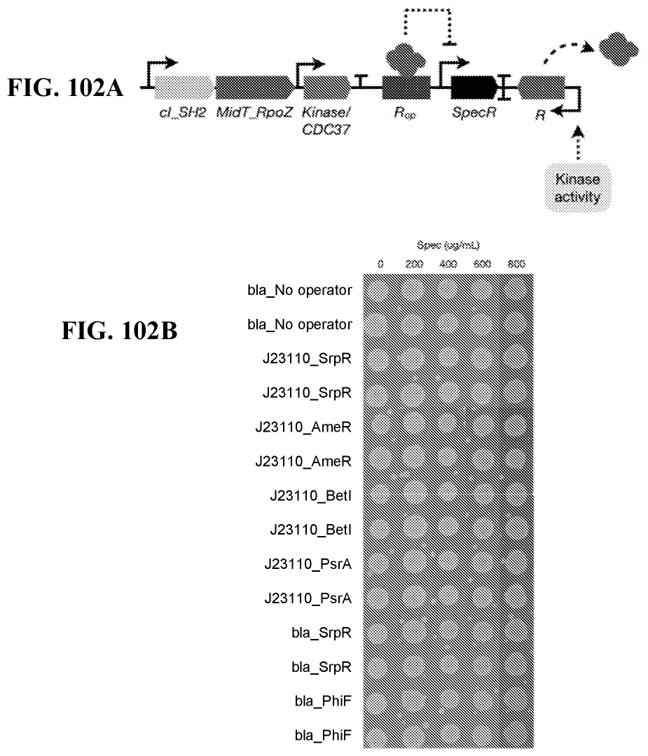

The inverted bacterial two-hybrid system was also developed that links kinase activity to the repression of a gene for spectinomycin resistance, as shown in FIG. 102A-102B. DH10BΔRpoω cells were produced harboring inverted two-hybrid systems with different combinations of SpecR promoters (bla or J23110) and repressors (SrpR, AmeR, BetI, PsrA, PhiF). In all constructs, repressors were paired with their cognate operator sequences. The “No operator” construct contains an HlyII repressor (R) with no operator sequence in the SpecR promoter. FIG. 102B suggests that the inverted two-hybrid system requires changes in expression of the repressor gene and/or the resistance gene.

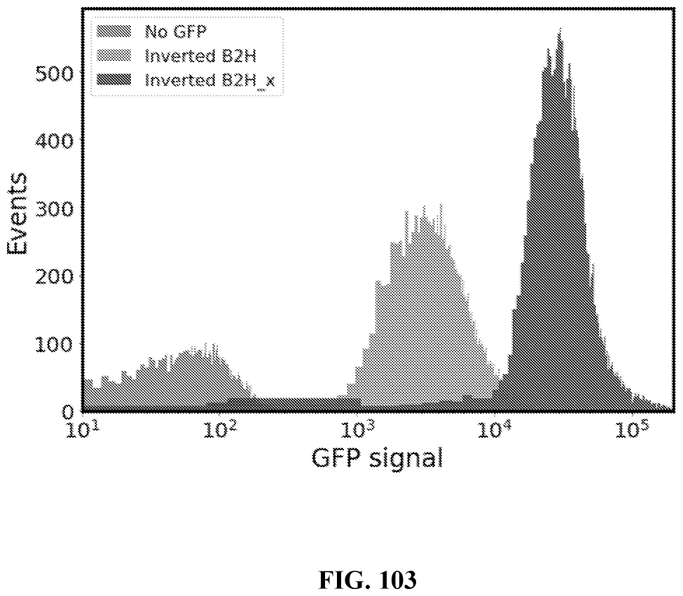

FIG. 103A provides histograms showing flow cytometry measurements of DH10BΔRpoω cells harboring three bacterial two-hybrid (B2H) systems: a negative control with no GFP (blue), an inverted B2H (light green, an inverted two-hybrid system that links kinase activity to the repression of a gene for spectinomycin resistance), inverted B2Hx (green, inverted B2H with the MidT Y/F mutation). Cells were gated to remove debris and to select for single cells. At least 10,000 events were collected for each measurement.

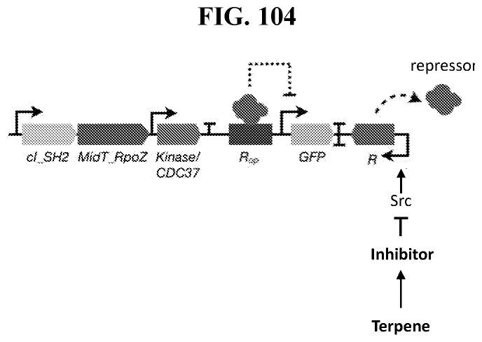

An inverted bacterial two-hybrid system was developed based on the phosphorylation dependent B2H system. E. Coli cells were engineered to express the Src Kinase Inverted B2H system in FIG. 104 against a library of individual terpene synthase enzymes. In some embodiments, agar plates containing spots of E. coli seeded from dops of liquid media were provided. These cells contain both (i) the inverted B2H system depicted in FIG. 104, (ii) a pathway that produces farnesyl pyrophosphate (pAM45), and (iii) a terpene synthase. The two top rows contain a catalytically inactive variant of amorphadiene synthase. The lower rows contain cells with various terpene synthases. After picking, the cells were grown overnight in LB media with antibiotics for plasmid maintenance. Cultures were subsequently diluted to an OD600 of ˜0.1 and 7 μL was spotted onto an LB-agar plate at pH 7.0 supplemented with 2% glycerol and allowed to grow for ˜48 h at room temperature. The merged image consists of an Alexa 488 channel and a colorimetric channel

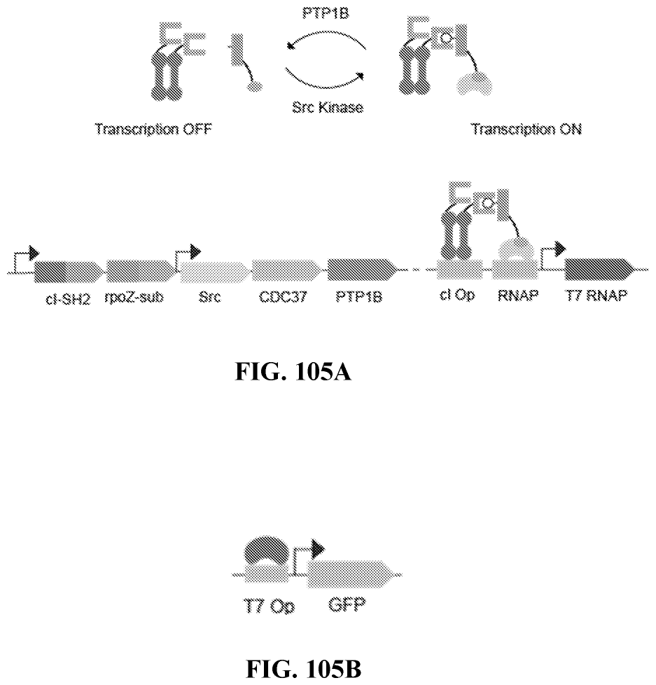

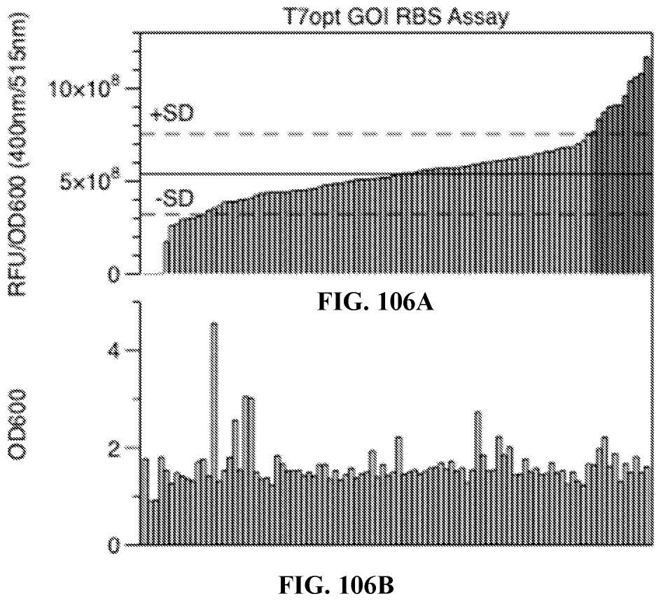

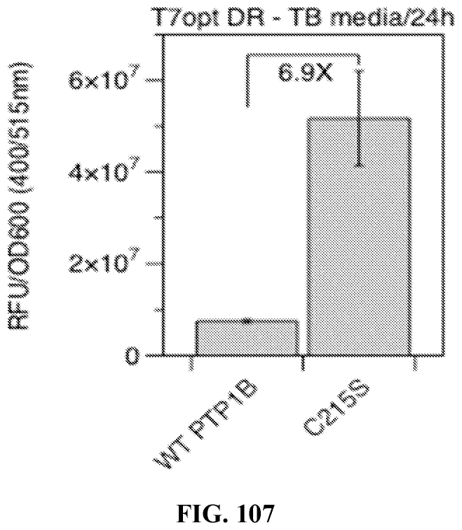

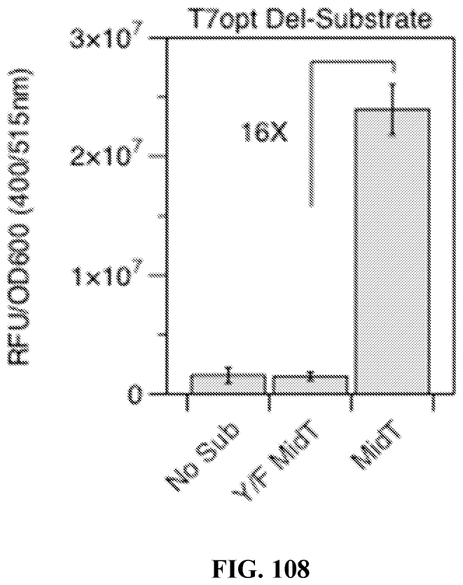

In other embodiments of the B2H system, a cI-SH2 fusion partner is expressed constitutively from a prol promoter (FIGS. 105A and 105B). In those embodiments, Src and chaperone, Cdc37, are expressed constitutively from the 327rod promoter. In those embodiments, T7 RNAP is the gene of interest, transcribed by recruitment of RNA polymerase via successful B2H partner binding. An auxiliary vector that supplied a rpoZ-sub fusion protein may be constitutively expressed from a prol promotor. In those embodiments, the expression of the rpoZ-sub fusion protein from the auxiliary vector restores transcriptional activity. In those embodiments, an auxiliary pET16b vector that provides GFPuv may be under control of the T7 operator (FIG. 105B). In those embodiments, the auxiliary pET16b vectors can be paired with expression of T7 RNAP via successful B2H partner binding to enable expression of GFPuv.

### Example 17: Development of B2H Systems with Alternative DNA Binding Domains

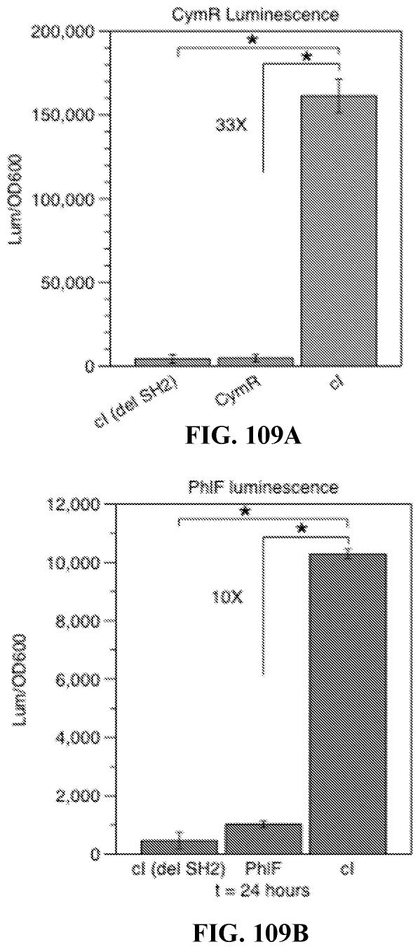

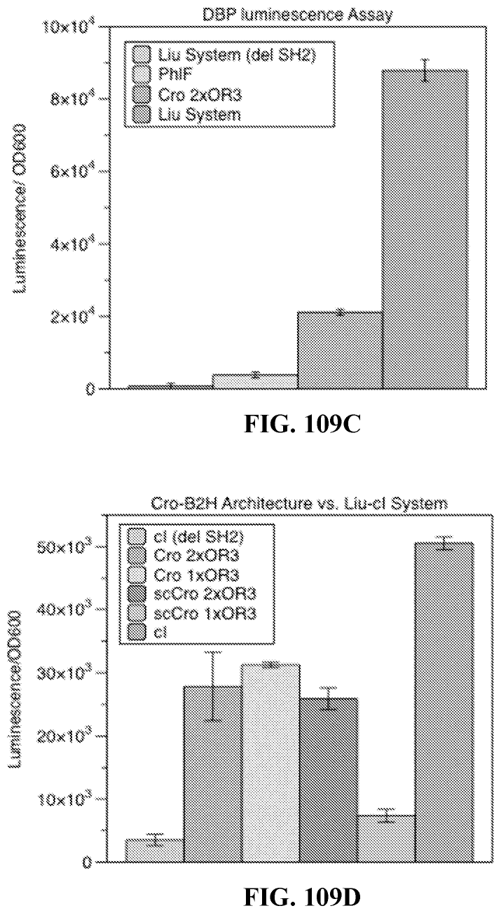

The B2H system has been developed to use different DNA Binding domains (FIG. 109). In FIG. 109A, DNA Binding Protein CymR-AM was inserted into a simple B2H system utilizing a DNA binding protein fused to an SH2 domain and the Omega Subunit of rpoZ fused to a HA4 monobody. CymR is localized to the promoter region of the gene of interest, LuxAB, by its operator CuO. Constitutive binding between HA4 and SH2 localizes RNA polymerase via RpoZ recruitment and initiates transcription of LuxAB. A luminescence assay of the CymR construct was conducted S1030 strain in TB media over a course of 8 hours. The original B2H construct containing cI was used as a reference condition, while the same construct with a deletion of SH2 domain was used as a negative control. These results show that CymR is a weaker initiator of transcription than cI, and illicits a signal that is not statistically significant than the leakiness exhibited by the negative control (*=P<0.05).

In FIG. 109B, DNA Binding Protein PhlF-AM was inserted into a simple B2H system utilizing a DNA binding protein fused to an SH2 domain and the Omega Subunit of rpoZ fused to a HA4 monobody. PhlF is localized to the promoter region of the gene of interest, LuxAB, by its operator PhlO. Constitutive binding between HA4 and SH2 localizes RNA polymerase via RpoZ recruitment and initiates transcription of LuxAB. A luminescence assay of the PhlF construct was conducted in S1030 strain in TB media over a course of 24 hours. The original B2H construct containing cI (a system using a phosphorylation-independent HA4-SH2 interaction instead of a phosphorylation-dependent MidT-SH2 interaction) was used as a reference condition, while the same construct with a deletion of SH2 domain was used as a negative control. These results show that PhlF is a weaker initiator of transcription than cI. (*=P<0.05)

In FIG. 109C, Lambda Phage DNA Binding Protein (DBP) Cro was also evaluated as a potential alternative to cI. The substitution of operator and DBP for that of Cro was made. The Liu system (cI, as previously described) and PhlF were used as reference points. Cro illicits a higher signal than PhlF but lower than cI as a DNA binding protein. In place of OR1/OR2 operators for cI in the original system, either one or two copies of OR3 (Cro's operator) were encoded. In the case of 1×OR3, the operator closest to the promoter (OR1) in the base system was substituted and the OR2 was deleted. In the case of 2×OR3, both cI operators were replaced with OR3. All cases were conducted in S1030 strain and cultured for 24 hours in TB media.

In FIG. 109D, Cro was followed up on for further study against the cI system. Different permutations to the system including numbers of operator and protein architecture were studied. In place of OR1/OR2 operators for cI in the original system, either one or two copies of OR3 (Cro's operator) were encoded. In the case of 1×OR3, the operator closest to the promoter (OR1) in the base system was substituted and the OR2 was deleted. In the case of 2×OR3, both cI operators were replaced with OR3. Two structural permutations of Cro were also examined. Cro produces a monomer of the protein which binds its operator as a dimer with itself. scCro creates a single chain of amino acids that encode two copies of Cro which remove the thermodynamic step associated with binding each other to form a dimer complex. In most cases, these permutations have little effect on transcriptional activity with exception to 1×OR3, scCro which exhibits the lowest luminescent signal. All cases were conducted in S1030 strain and cultured in TB media for a course of 24 hours.

### Example 18: Development of a Light Sensitive B2H System

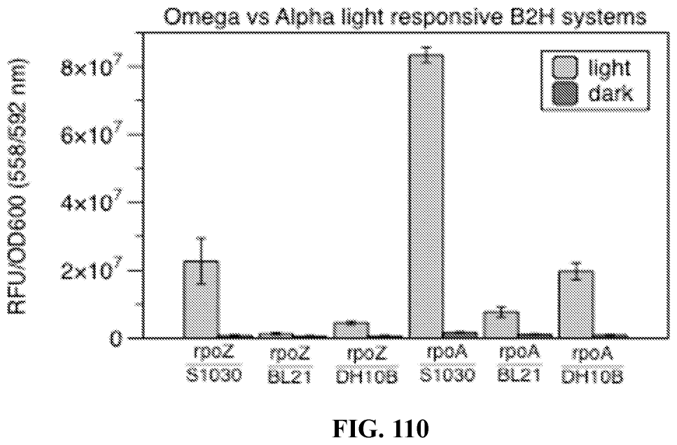

An experiment using a B2H system using iLID-SsrA/SspB as binding partners was used to interrogate the effect of an rpoA substitution on transcriptional activity in multiples strains of E. coli. Red fluorescent protein, mRuby3, was the gene of interest as a reporter of transcriptional activation (FIG. 110). Transcriptional activity was induced in all cases by exposing cultures to 490 nm blue light for 24 hours to enable SsrA-SspB binding, thereby localizing rpoZ or rpoA to the promoter site. All 1 mL cultures were grown in LB media for 24 hours in a clear 24 well plate. In FIG. 110, substitution of alpha subunit, rpoA, enables significant activation of the B2H system in strains without a genomic deletion of the omega subunit, rpoZ. In strains not containing a deletion to the Omega subunit (BL21 and DH10B), B2H system still utilizing rpoZ as its RNAP recruiter exhibit poor transcriptional activation.

### Example 19: Development of a B2H System Linking TCPTP Inactivation to Spectinomycin Resistance

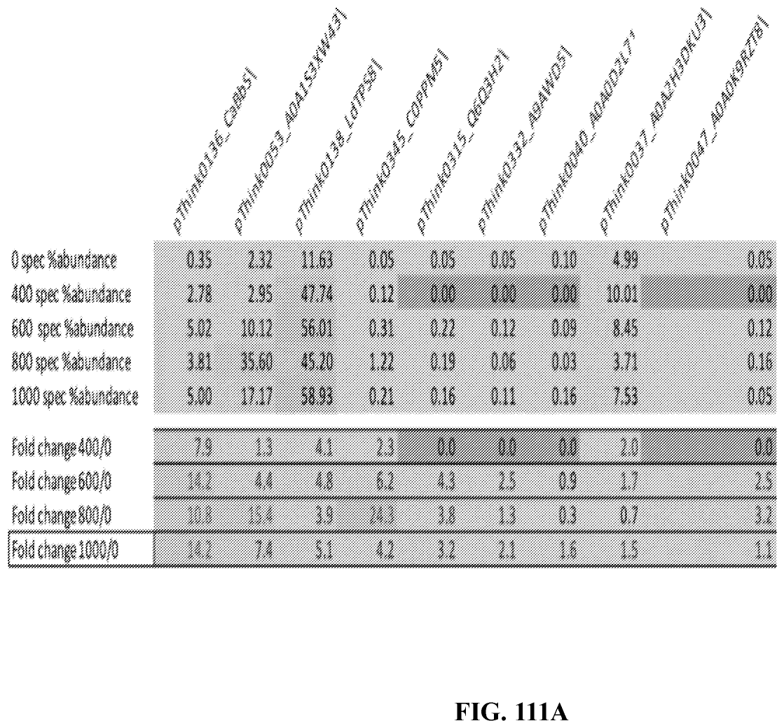

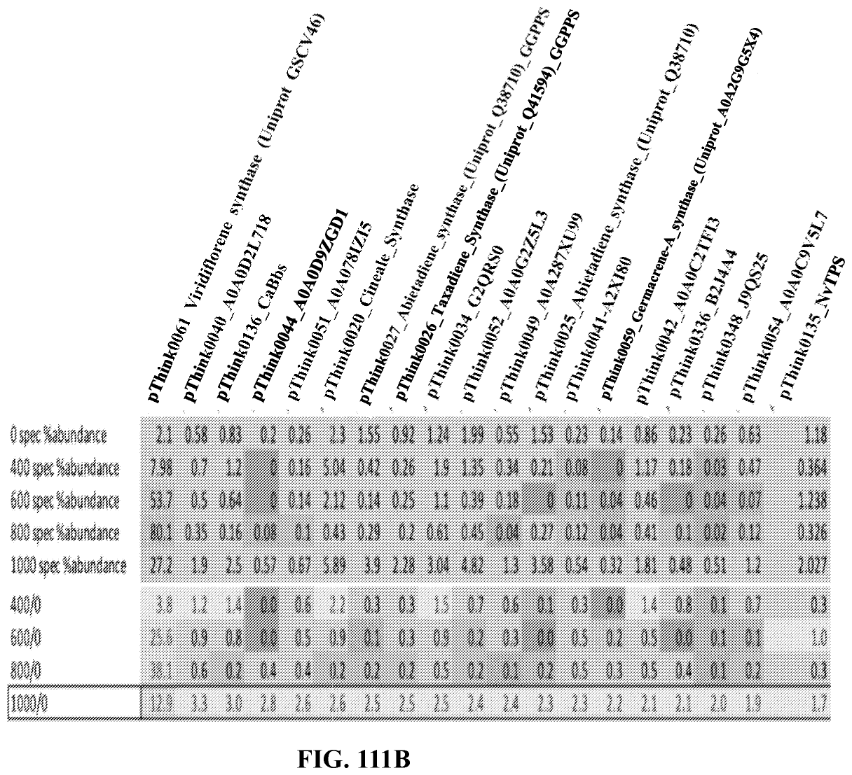

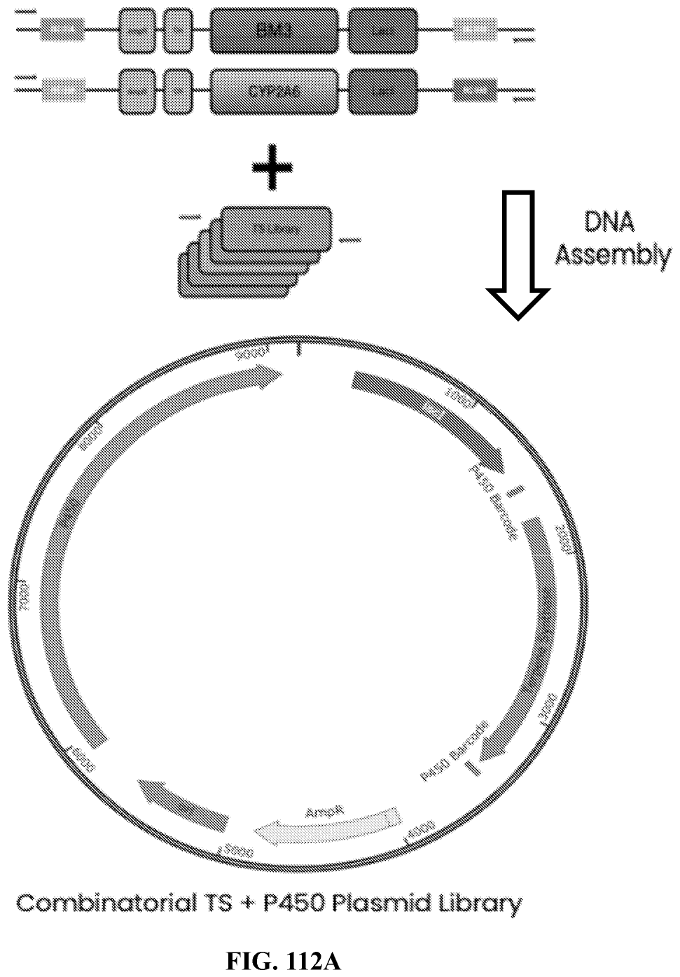

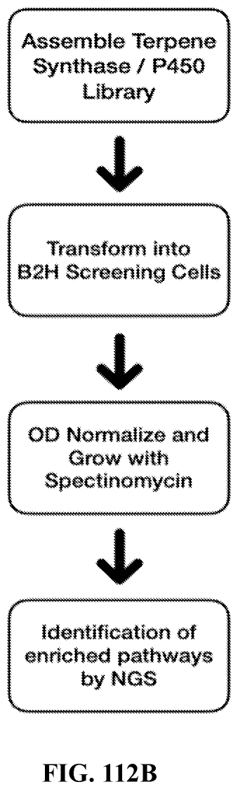

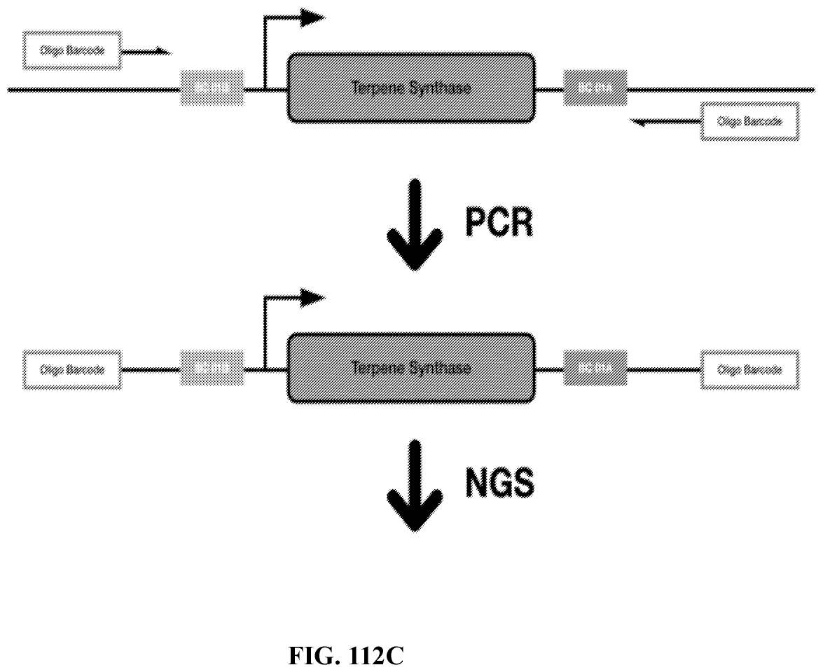

A next-generation sequencing experiment carried out in E. coli cells harboring a B2H system that links PTP1B inactivation to the expression of gene for spectinomycin resistance and a terpenoid pathway, comprising an isoprenoid pathway (pAM45), a terpene synthase, and a cytochrome P450 (CYP2A6) (FIG. 111A). In this experiment, the cells were seeded on agar plates with different concentrations of spectinomycin. NGS was used to assess the population fraction associated with different terpenoid pathways. In an analogous experiment (FIG. 111B), the B2H linked TCPTP inactivation to the expression of a gene for spectinomycin resistance.

### Example 20: Selection of B2H Systems Using Mutated PTP1B Linked to GFP

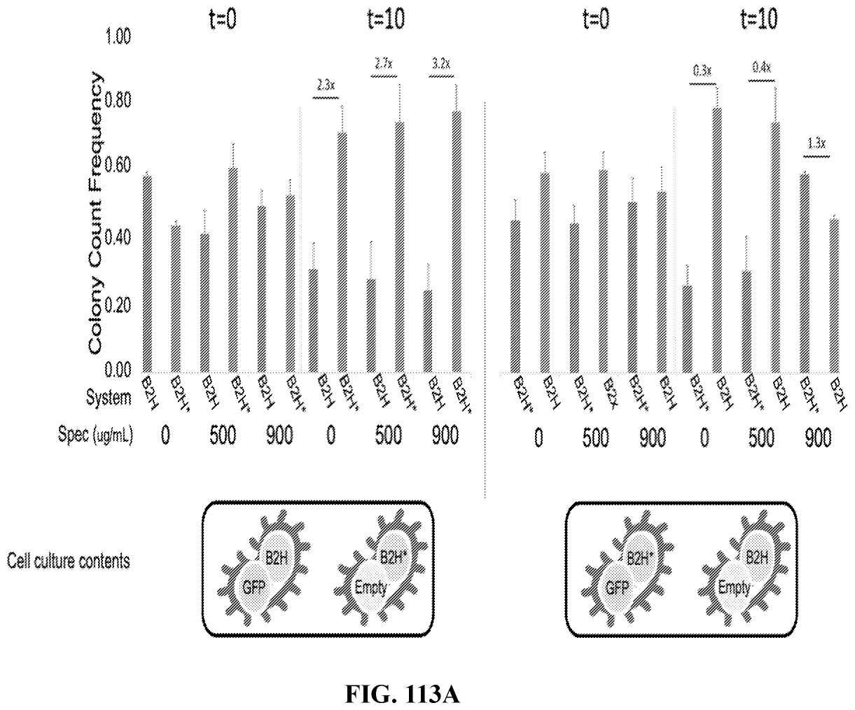

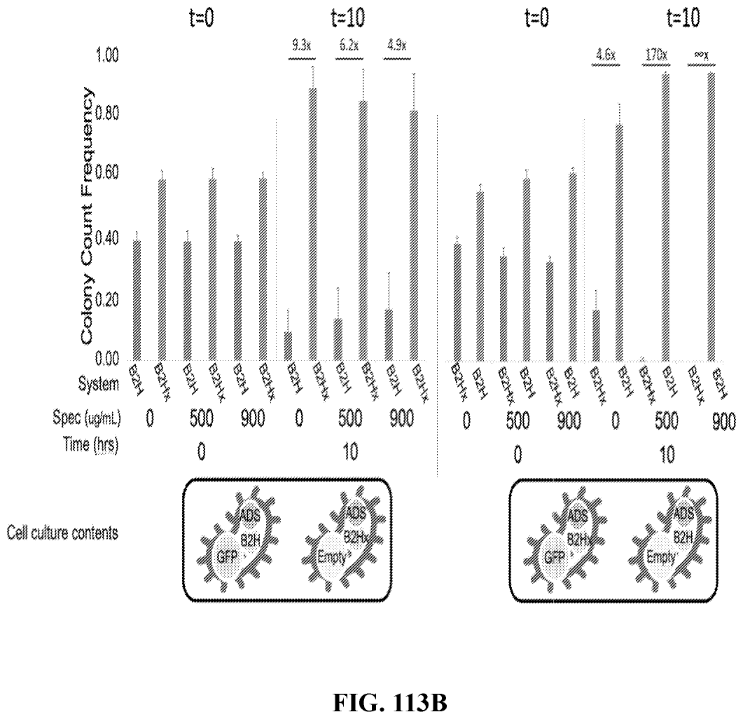

Selection experiments carried out with two strains of E. coli were grown in liquid media in the presence of spectinomycin (LB with antibiotics for plasmid maintenance and concentrations of spectinomycin as indicated) (FIG. 113). The γ-axis plots the population fraction belonging to each strain, as assessed by counting colonies on agar plates seeded from liquid culture at various time points.

FIG. 113A (left) shows a first strain of E. coli (B2H) that contained both a gene for GFP and a B2H system that links PTP1B inactivation to the expression of gene for spectinomycin resistance, and a second strain (B2H*) contained both an empty plasmid identical to the one carrying GFP in the first strain and a B2H system with a catalytically inactive (C215S) mutant of PTP1B. High concentrations of spectinomycin and longer growth times appear to enrich for the B2H* expressing strain. FIG. 113A (right) describes the complementary experiment in which the first strain (B2H*) has both GFP and a B2H system with the C215S mutant of PTP1B, and the second strain (B2H) has both an empty vector and a B2H system. High concentrations of spectinomycin and long growth times still appear to enrich for the second strain, which has B2H but, perhaps, less so than in the first experiment. In both experiments, the numbers above the bars show the population fraction of B2H* divided by the population fraction of B2H.

FIG. 113B (left) shows a first strain of E. coli (B2H) contained both a gene for GFP, a B2H system that links PTP1B inactivation to the expression of gene for spectinomycin resistance, and a gene for amorphadiene synthase (ADS). The second strain (B2Hx) contained an empty plasmid identical to the one carrying GFP in the first strain, the B2Hx system that includes a B2H system in which a Y/F mutation in the substrate domain (MidT) prevents its phosphorylation that helps it bind to the receptor, and a gene encoding ADS. FIG. 113B (right) describes the complementary experiment in which the first strain (B2Hx) has both GFP, B2Hx system, and ADS. The second strain (B2H) contained an empty vector, a B2H system, and ADS. High concentrations of spectinomycin and long growth times still appear to enrich for the second strain, which has B2H but, perhaps, less so than in the first experiment. In both experiments, the numbers above the bars show the population fraction of B2H* divided by the population fraction of B2H.

### Example 21: Development of B2H Systems Linking Terpenoid Pathways to Spectinomycin

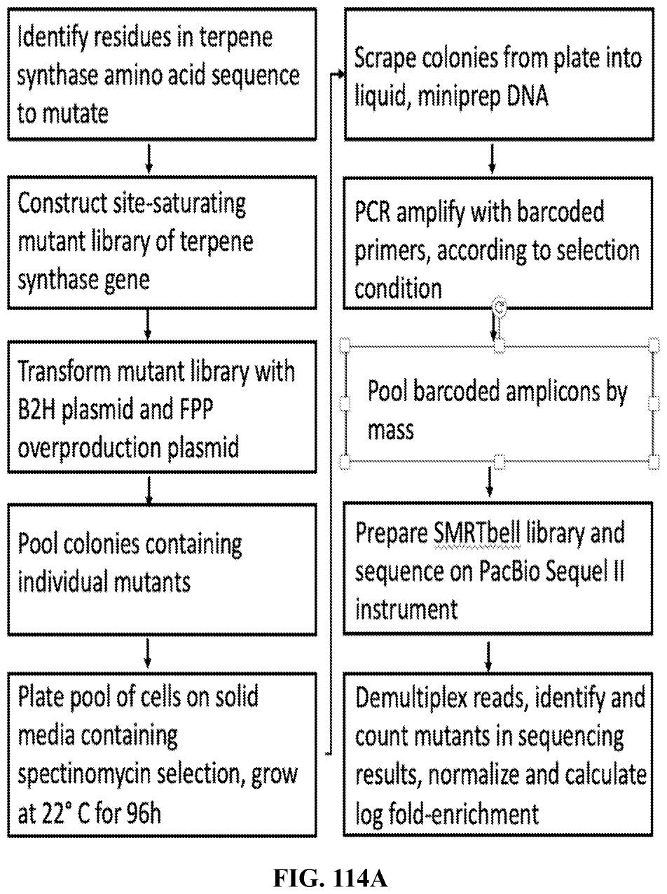

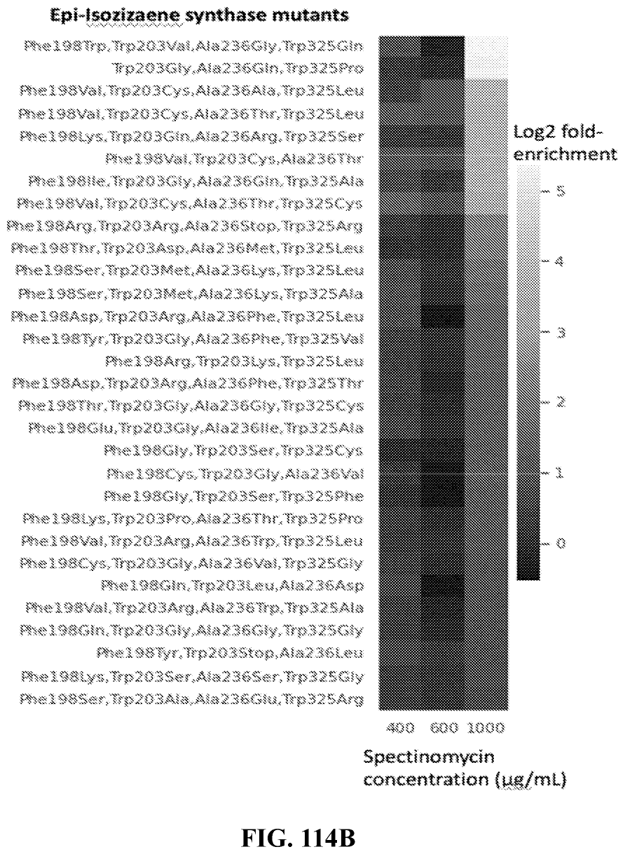

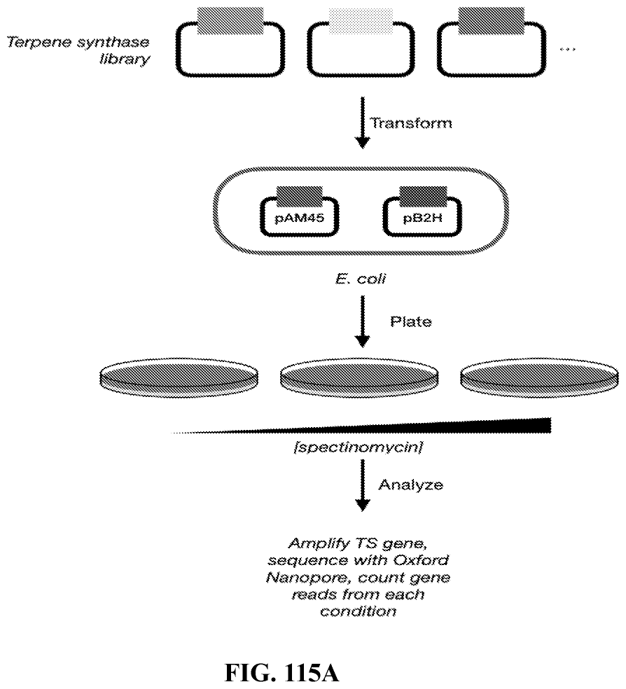

Next generation sequencing was used to identify terpenoid pathways that confer a survival advantage under spectinomycin selection (FIG. 114). FIG. 114A depicts a schematic describing the construction and screening of a terpene synthase mutant library against a protein target of interest in a B2H experiment. In brief, the terpene synthase gene is mutated at amino acid residues of interest and the resulting library is co-transformed into E. coli DH10BΔRpoω along with a plasmid containing the B2H system and a plasmid containing the biosynthetic pathway necessary to overproduce farnesyl pyrophosphate (FPP) (pAM45). The E. coli colonies containing these plasmids are pooled and spread onto solid media containing different concentrations spectinomycin, at different selection levels. After 96 h of growth, the resulting colonies are pooled via plate scraping and the DNA is extracted through plasmid miniprep. This DNA is used as a template in a PCR reaction, in which the PCR primers contain DNA barcodes corresponding to growth condition. The barcoded PCR libraries are then pooled and prepped in SMRTbell format for PacBio sequencing. The sequenced subreads were used to generate consensus sequences for each read and subsequently demultiplexed according to each selection condition. The unselected (0 μg/mL spectinomycin) was then used as a normalization condition for the calculation of enrichment scores for each variant.

FIG. 114B shows the next generation sequencing results from the mutagenesis and screening of Epi-Isozizaene synthase. Epi-Isozizaene synthase mutants identified from PacBio HiFi sequencing results are ranked by log 2 fold-enrichment with respect to the unselected (0 μg/mL) population. Here, the 30 mutants with the highest enrichment comparing 1000 μg/mL spectinomycin population to the unselected population are shown. Individual subreads from the PacBio Sequel II sequencing run were combined into one fastq file using the ccs package. Barcoded reads were demultiplexed and filtered for ≥Q30 using lima. Mutations were identified and counted for each condition using alignparse.

### Example 22: An NGS Screen of Terpene Synthases

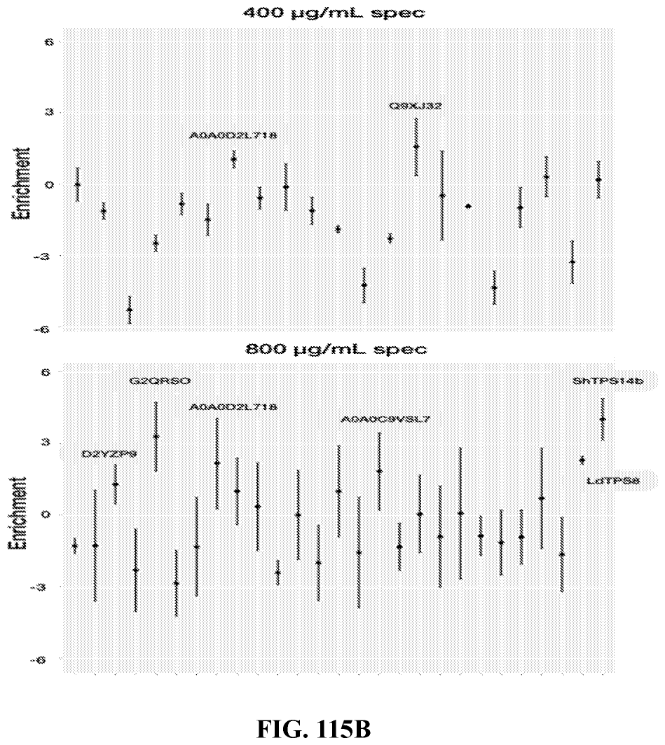

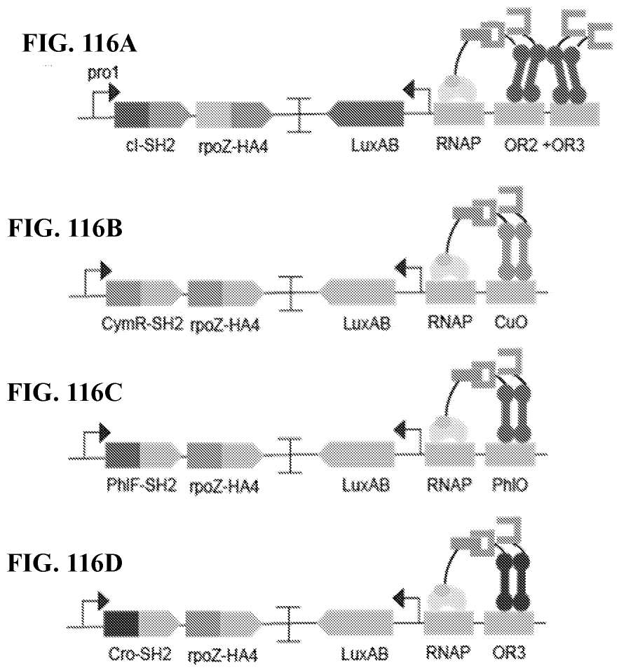

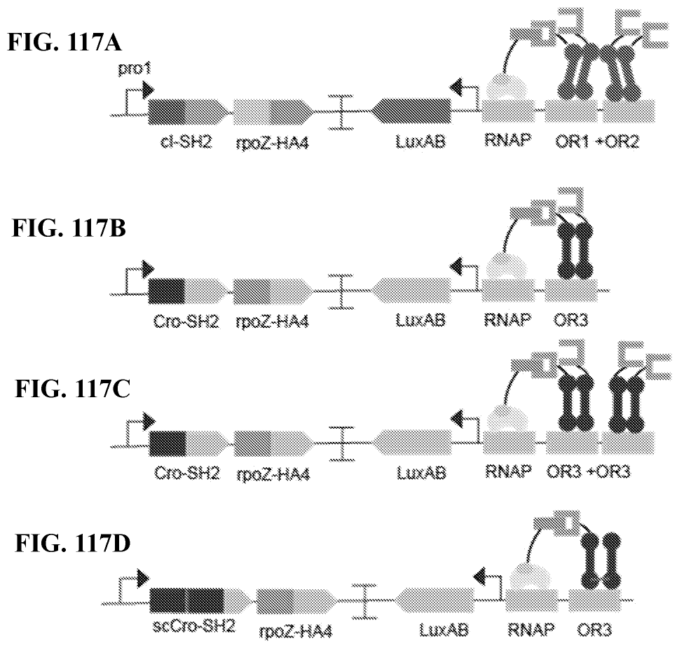

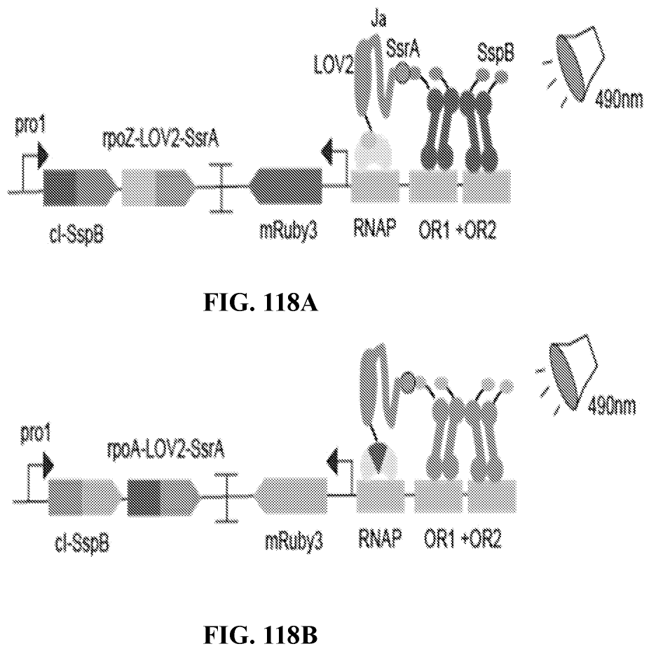

FIG. 116A-116B show an embodiment of a workflow for NGS-based screening of terpene synthases. In FIG. 116A, a plasmid-borne library of terpene synthases are transformed into E. coli cells harboring a terpene precursor (pAM45) and detection (pB2H) plasmids. Transformants are recovered and plated on solid media containing increasing concentrations of the antibiotic spectinomycin. The resulting cells are grown and then sequenced in bulk to obtain distributions of terpene synthase genes within each population. Enrichment from 0 μg/mL spectinomycin to higher concentrations is computed as log 2((ci/Ni)/(c0/N0)), where ci is the counts of an individual terpene synthases gene and Ni is the sum of counts, both at a given spectinomycin concentration. FIG. 118B shows the workflow depicted in FIG. 118A carried out in triplicate using a PTPRC-based B2H detection system. Genes with enrichment values >0 are labeled. Error bars in FIG. 118B denote standard error of N=3 biological replicates.

### Example 23. Detecting Activators of the Target Enzyme

The two-hybrid system may be used to detect a presence of bioactive molecules that enhance activity of the target enzyme, instead of inhibitors of the target enzyme. This may be accomplished using the same two-hybrid system described elsewhere herein (see for e.g., Examples 1-22) by measuring a decrease in expression of the gene of interest (GOI) rather than an increase in expression of the GOI relative to a reference expression level. A reference level may be obtained from an otherwise identical cell that does not comprise a functional metabolic pathway that produces the bioactive molecule, the ligand or the receptor.

### Example 24. Detecting Modulators of a Kinase

The two-hybrid system may be used to detect a presence of a bioactive molecule that modulates activity of a kinase. A phosphate-dependent two-hybrid system described elsewhere herein can be used to detect a presence of a bioactive molecule that enhances activity of a kinase. In each case, you would expect an increase in expression of the gene of interest (GOI).

An inverted phosphate-dependent two-hybrid system described in Example 16 can be used to detect a presence of a bioactive molecule that inhibits activity of a kinase. Inhibition of a kinase will prevent the kinase from phosphorylating the kinase substrate, thereby preventing the kinase substrate from binding to the phosphorylated protein binding domain. Without formation of the kinase substrate-phosphorylated protein binding domain pair, transcriptional activation of the gene of interest does not occur, thereby increasing expression of a reporter polypeptide (that is inversely correlated with the expression of the GOI).

What is claimed is:

1. A method for performing multiplexed discovery of bioactive molecules that modulate activity of a target enzyme, the method comprising:
(a) providing a plurality of cells;
(b) introducing into each of the plurality of cells a synthetic genetically-encoded system that links expression of a gene of interest to biosynthesis of a bioactive molecule by a cell of the plurality of cells, wherein the synthetic genetically-encoded system encodes the target enzyme, a gene of interest, a synthase of the bioactive molecule, a ligand, and a receptor specific to the ligand under conditions sufficient for binding of the ligand to the receptor to produce a ligand-receptor pair, wherein the ligand-receptor pair activates transcription of the gene of interest;
(c) performing multiplexed sequencing of the plurality of cells; and
(d) identifying a subset of the plurality of cells in which the expression of the gene of interest is increased relative to a reference expression level, wherein the reference expression level is obtained from an otherwise identical reference cell that does not comprise a metabolic pathway that produces the bioactive molecule, the ligand or the receptor.

2. The method of claim 1, wherein the expression of the gene of interest is increased relative to the reference expression level when the bioactive molecule is present in the cell at concentrations sufficient to modulate the activity of the target enzyme.

3. The method of claim 1 or 2, wherein modulation of the target enzyme by the bioactive molecule disrupts binding between the receptor and the ligand, thereby reducing transcriptional activation of the gene of interest.

4. The method of any one of claims 1 to 3, wherein the binding of the ligand to the receptor is phosphorylation dependent.

5. The method of any one of claims 1 to 4, wherein the plurality of cells are prokaryotic cells.

6. The method of claim 5, wherein the prokaryotic cells comprise bacterial cells.

7. The method of any one of claims 1 to 6, wherein the bioactive molecule comprises a terpenoid.

8. The method of any one of claims 1 to 7, wherein the target enzyme comprises a proteolytic enzyme, a phosphatase, or a kinase.

9. The method of claim 8, wherein the phosphatase comprises a tyrosine phosphatase.

10. The method of claim 8 or 9, wherein the kinase comprises a tyrosine kinase.

11. The method of any one of claims 1 to 10, wherein the exogenous genetically-encoded system comprises a two-hybrid system encoding the target enzyme, the gene of interest, the ligand, and the receptor.

12. The method of any one of claims 1 to 11, wherein the synthase comprises a terpene synthase or a nonribosomal peptide synthetase.

13. The method of claim 12, wherein the terpene synthase comprises γ-humulene synthase (GHS), amorphadiene synthase (ADS), α-bisabolene synthase (ABS), or taxadiene synthase (TXS).

14. The method of claim 12 or 13, wherein the terpene synthase comprises an amino acid sequence that is greater than or equal to about 90% identical to any one of SEQ ID NO: 4, 7, 9, 11, 13, 15, 17, 19, or 23.

15. The method of any one of claims 1 to 14, wherein the ligand comprises a kinase substrate that binds to the receptor in a phosphorylated state, and the receptor comprises a phosphorylated protein binding domain that binds to the kinase substrate when the kinase substrate is phosphorylated.

16. The method of claim 15, wherein the ligand is coupled to a subunit of RNA polymerase and the receptor is coupled to a DNA binding domain, or the ligand is coupled to the DNA binding domain and the receptor is coupled to the omega subunit of the RNA polymerase.

17. The method of any one of claims 1 to 16, wherein the gene of interest encodes a reporter polypeptide comprising a luciferase enzyme, a fluorescent polypeptide, secreted alkaline phosphatase, ß-galactosidase levansucrase, chloramphenicol acetyltransferase (CAT), or a protein that confers antibiotic resistance.

18. The method of any one of claims 1 to 17, wherein the gene of interest encodes a polymerizing enzyme that, when expressed, binds to a promoter operably linked to a gene encoding the reporter polypeptide to drive expression of the reporter polypeptide.

19. The method of claim 18, wherein the expression of the reporter polypeptide from the gene is greater than an expression of the reporter polypeptide if it were encoded by the gene of interest.

20. The method of claim 19, wherein the expression of the reporter polypeptide is greater by more than or equal to about 2-fold.

21. The method of any one of claims 1 to 20, wherein the genetically-encoded system further encodes a metabolic pathway for biosynthesis of the bioactive molecule.

22. The method of claim 21, wherein the metabolic pathway is an isoprenoid pathway.

23. The method of claim 22, wherein the isoprenoid pathway comprises a mevalonate pathway, a methylerthritol 4-phosphate (MEP) pathway, a deoxyxylulose 5-phosphate (DXP) pathway, or an isopentenol utilization (IUP) pathway.

24. The method of any one of claims 1 to 23, wherein the multiplex sequencing comprises long read sequencing.

25. The method of claim 24, Wherein the synthetic genetically-encoded system comprises one or more molecular barcode sequences that uniquely identifies the target enzyme, the synthase, or a combination thereof.

26. The method of claim 25, wherein the multiplex sequencing further comprises performing demultiplexing, thereby assigning each of the one or more molecular barcodes with the target enzyme, the synthase, or the combination thereof, for each cell of the subset of the plurality of cells.

27. The method of any one of claims 1 to 26, further comprising performing multiplexed sequencing of the plurality of cells prior to introducing in (b), wherein the identifying in (d) comprises detecting enrichment of the gene of interest following the introducing in (b).

28. A system, comprising:
one or more nucleic acid molecules encoding a genetically-encoded system that, when expressed in a cell, links expression of a gene of interest to biosynthesis by the cell of a bioactive molecule that modulates activity of a target enzyme, wherein the genetically-encoded system comprises:
the target enzyme;
a synthase of the bioactive molecule;
a ligand; and
a receptor specific to the ligand,
wherein (i) the ligand is coupled to a subunit of RNA polymerase and the receptor is coupled to a DNA binding protein, or (ii) the ligand is coupled to the DNA binding protein and the receptor is coupled to the subunit of RNA polymerase, and
wherein the one or more nucleic acid molecules comprises:
one or more adaptor molecules comprising a sequencing primer binding site;
the gene of interest; and
a transcription initiation site for the gene of interest comprising:
a binding site for the DNA binding protein; and
a promoter sequence comprising a binding site for the RNA polymerase.

29. The system of claim 28, further comprising the cell comprising the one or more nucleic acid molecules.

30. The system of claim 29, wherein the cell is a prokaryotic cell.

31. The system of claim 20, wherein the prokaryotic cell comprises a bacterial cell.

32. The system of any one of claims 29 to 31, wherein the cell is isolated.

33. The system of any one of claims 28 to 32, wherein the bioactive molecule comprises a terpenoid.

34. The system of any one of claims 28 to 33, wherein the target enzyme comprises a proteolytic enzyme, a phosphatase, or a kinase.

35. The system of claim 34, wherein the phosphatase comprises a tyrosine phosphatase.

36. The system of claim 34 or 35, wherein the kinase comprises a tyrosine kinase.

37. The system of any one of claims 28 to 36, wherein the subunit of the RNA polymerase comprises an omega subunit of the RNA polymerase.

38. The system of any one of claims 28 to 37, wherein the exogenous genetically-encoded system comprises a two-hybrid system encoding the target enzyme, the ligand, the receptor, and the gene of interest.

39. The system of any one of claims 28 to 38, wherein the synthase comprises a terpene synthase or a nonribosomal peptide synthetase.

40. The system of claim 39, wherein the terpene synthase comprises γ-humulene synthase (GHS), amorphadiene synthase (ADS), α-bisabolene synthase (ABS), or taxadiene synthase (TXS).

41. The system of claim 39 or 40, wherein the terpene synthase comprises an amino acid sequence that is greater than or equal to about 90% identical to any one of SEQ ID NO: 4, 7, 9, 11, 13, 15, 17, 19, or 23.

42. The system of any one of claims 28 to 41, wherein the ligand comprises a kinase substrate that binds to the receptor in a phosphorylated state, and the receptor comprises a phosphorylated protein binding domain that binds to the kinase substrate when the kinase substrate is phosphorylated.

43. The system of claim 42, wherein the ligand is coupled to a subunit of RNA polymerase and the receptor is coupled to a DNA binding domain, or the ligand is coupled to the DNA binding domain and the receptor is coupled to the subunit of the RNA polymerase.

44. The system of any one of claims 28 to 43, wherein the gene of interest encodes a reporter polypeptide comprising a luciferase enzyme, a fluorescent polypeptide, secreted alkaline phosphatase, ß-galactosidase levansucrase, chloramphenicol acetyltransferase (CAT), or a protein that confers antibiotic resistance.

45. The system of any one of claims 28 to 44, wherein the gene of interest encodes a modulator protein that is operably linked to a gene encoding the reporter polypeptide, wherein the modulator protein activates or represses expression of the reporter polypeptide.

46. The system of any one of claims 28 to 45, wherein the one or more adaptor molecules comprises one or more molecular barcode sequences unique to the target enzyme, the synthase, or the combination thereof.

47. The system of any one of claims 28 to 46, wherein the genetically-encoded system further encodes a metabolic pathway for biosynthesis of the bioactive molecule.

48. The system of claim 47, wherein the metabolic pathway is an isoprenoid pathway.

49. The system of claim 48, wherein the isoprenoid pathway comprises a mevalonate pathway, a methylerthritol 4-phosphate (MEP) pathway, a deoxyxylulose 5-phosphate (DXP) pathway, or an isopentenol utilization (IUP) pathway.

50. The system of any one of claims 47 to 49, wherein the one or more adaptor molecules further comprises another barcode sequence unique to the metabolic pathway.

51. A method of determining a presence of a bioactive molecule that modulates activity of a target enzyme, the method comprising:
(a) introducing into a cell a synthetic genetically-encoded system that links expression of a gene of interest to biosynthesis of the bioactive molecule by the cell, wherein the synthetic genetically-encoded system encodes the target enzyme, a gene of interest encoding modulatory protein that modulates expression of a reporter polypeptide, the reporter polypeptide, a synthase of the bioactive molecule, a ligand, and a receptor specific to the ligand under conditions sufficient for binding of the ligand to the receptor to form a ligand-receptor pair, wherein the ligand-receptor pair activates transcription of the gene of interest;
(b) measuring the expression of the reporter polypeptide; and
(c) determining the presence of the bioactive molecule in the cell if the expression of the reporter polypeptide is increased or decreased relative to a reference expression level obtained from an otherwise identical reference cell that does not comprise a functional metabolic pathway that produces the bioactive molecule, the ligand or the receptor.

52. The method of claim 51, wherein the expression of the reporter polypeptide is increased relative to the reference expression level when the bioactive molecule is present in the cell at concentrations sufficient to modulate the activity of the target enzyme.

53. The method of claim 52, wherein the modulatory protein comprises a polymerizing enzyme that activates transcription of the reporter polypeptide.

54. The method of any one of claims 51 to 53, wherein the expression of the reporter polypeptide is decreased relative to the reference expression level when the bioactive molecule is present in the cell at concentrations sufficient to modulate the activity of the target enzyme.

55. The method of claim 54, wherein the modulatory protein comprises a transcriptional repressor that represses transcription of the reporter polypeptide.

56. The method of any one of claims 51 to 55, wherein modulation of the target enzyme by the bioactive molecule disrupts binding between the receptor and the ligand, thereby reducing transcriptional activation of the gene of interest.

57. The method of any one of claims 51 to 56, wherein the binding of the ligand to the receptor is phosphorylation dependent.

58. The method of any one of claims 51 to 57, wherein cell is a prokaryotic cell.

59. The method of claim 58, wherein the prokaryotic cell is a bacterial cell.

60. The method of any one of claims 51 to 59, wherein the bioactive molecule comprises a terpenoid.

61. The method of any one of claims 51 to 60, wherein the target enzyme comprises a proteolytic enzyme, a phosphatase, or a kinase.

62. The method of claim 61, wherein the phosphatase comprises a tyrosine phosphatase.

63. The method of claim 61 or 62, wherein the kinase comprises a tyrosine kinase.

64. The method of any one of claims 51 to 63, wherein the exogenous genetically-encoded system comprises a two-hybrid system encoding the target enzyme, the ligand, the receptor, and the gene of interest.

65. The method of any one of claims 51 to 64, wherein the synthase comprises a terpene synthase or a nonribosomal peptide synthetase.

66. The method of claim 64, wherein the terpene synthase comprises γ-humulene synthase (GHS), amorphadiene synthase (ADS), α-bisabolene synthase (ABS), or taxadiene synthase (TXS).

67. The method of claim 64 or 65, wherein the terpene synthase comprises an amino acid sequence that is greater than or equal to about 90% identical to any one of SEQ ID NO: 4, 7, 9, 11, 13, 15, 17, 19, or 23.

68. The method of any one of claims 51 to 67, Wherein the ligand comprises a kinase substrate that binds to the receptor in a phosphorylated state, and the receptor comprises a phosphorylated protein binding domain that binds to the kinase substrate when the kinase substrate is phosphorylated.

69. The method of claim 68, wherein the ligand is coupled to a subunit of RNA polymerase and the receptor is coupled to a DNA binding domain, or the ligand is coupled to the DNA binding domain and the receptor is coupled to the subunit of the RNA polymerase.

70. The method of any one of claims 51 to 69, Wherein the reporter polypeptide comprises a luciferase enzyme, a fluorescent polypeptide, secreted alkaline phosphatase, ß-galactosidase levansucrase, chloramphenicol acetyltransferase (CAT), or a protein that confers antibiotic resistance.

71. The method of any one of claims 51 to 70, wherein the genetically-encoded system further encodes a metabolic pathway for biosynthesis of the bioactive molecule.

72. The method of claim 71, wherein the metabolic pathway is an isoprenoid pathway.

73. The method of claim 72, wherein the isoprenoid pathway comprises a mevalonate pathway, a methylerthritol 4-phosphate (MEP) pathway, a deoxyxylulose 5-phosphate (DXP) pathway, or an isopentenol utilization (IUP) pathway.

74. A system, comprising:
one or more nucleic acid molecules encoding a genetically-encoded system that, when expressed in a cell, links expression of a gene of interest to biosynthesis by the cell of a bioactive molecule that modulates activity of a target enzyme, wherein the genetically-encoded system comprises:
a reporter polypeptide;
the target enzyme;
a synthase of the bioactive molecule;
a ligand; and
a receptor specific to the ligand, wherein (i) the ligand is coupled to a subunit of RNA polymerase and the receptor is coupled to a DNA binding protein, or (ii) the ligand is coupled to the DNA binding protein and the receptor is coupled to the subunit of RNA polymerase, and
wherein the one or more nucleic acid molecules comprises:
the gene of interest, wherein the gene of interest encodes a modulator protein configured to activate transcription or repress transcription of the reporter polypeptide; and
a transcription initiation site for the gene of interest comprising:
a binding site for the DNA binding protein; and
a promoter sequence comprising a binding site for the RNA polymerase.

75. The system of claim 74, wherein the modulatory protein comprises a polymerizing enzyme that activates transcription of the reporter polypeptide.

76. The system of claim 74 or 75, wherein the modulatory protein comprises a transcriptional repressor that represses transcription of the reporter polypeptide.

77. The system of any one of claims 74 to 76, further comprising the cell comprising the one or more nucleic acid molecules.

78. The system of claim 77, wherein the cell is a prokaryotic cell.

79. The system of claim 78, wherein the prokaryotic cell comprises a bacterial cell.

80. The system of any one of claims 77 to 79, wherein the cell is isolated.

81. The system of any one of claims 74 to 80, wherein the bioactive molecule comprises a terpenoid.

82. The system of any one of claims 74 to 81, wherein the target enzyme comprises a proteolytic enzyme, a phosphatase, or a kinase.

83. The system of claim 82, wherein the phosphatase comprises a tyrosine phosphatase.

84. The system of claim 82 or 83, wherein the kinase comprises a tyrosine kinase.

85. The system of any one of claims 74 to 84, wherein the subunit of the RNA polymerase comprises an omega subunit of the RNA polymerase.

86. The system of any one of claims 74 to 85, wherein the exogenous genetically-encoded system comprises a two-hybrid system encoding the target enzyme, the ligand, the receptor, and the gene of interest.

87. The system of any one of claims 74 to 86, wherein the synthase comprises a terpene synthase or a nonribosomal peptide synthetase.

88. The system of claim 87, wherein the terpene synthase comprises γ-humulene synthase (GHS), amorphadiene synthase (ADS), α-bisabolene synthase (ABS), or taxadiene synthase (TXS).

89. The system of claim 87 or 88, wherein the terpene synthase comprises an amino acid sequence that is greater than or equal to about 90% identical to any one of SEQ ID NO: 4, 7, 9, 11, 13, 15, 17, 19, or 23.

90. The system of any one of claims 74 to 89, wherein the ligand comprises a kinase substrate that binds to the receptor in a phosphorylated state, and the receptor comprises a phosphorylated protein binding domain that binds to the kinase substrate when the kinase substrate is phosphorylated.

91. The system of claim 90, wherein the ligand is coupled to a subunit of RNA polymerase and the receptor is coupled to a DNA binding domain, or the ligand is coupled to the DNA binding domain and the receptor is coupled to the subunit of the RNA polymerase.

92. The system of any one of claims 74 to 91, wherein the reporter polypeptide comprises a luciferase enzyme, a fluorescent polypeptide, secreted alkaline phosphatase, ß-galactosidase levansucrase, chloramphenicol acetyltransferase (CAT), or a protein that confers antibiotic resistance.

93. The system of any one of claims 74 to 92, wherein the genetically-encoded system further encodes a metabolic pathway for biosynthesis of the bioactive molecule.

94. The system of claim 93, wherein the metabolic pathway is an isoprenoid pathway.

95. The system of claim 94, wherein the isoprenoid pathway comprises a mevalonate pathway, a methylerthritol 4-phosphate (MEP) pathway, a deoxyxylulose 5-phosphate (DXP) pathway, or an isopentenol utilization (IUP) pathway.

96. The system of any one of claims 93 to 95, wherein the one or more nucleic acid molecules further comprises one or more barcode sequences unique to the metabolic pathway.

97. The system of any one of claims 74 to 96, wherein the one or more nucleic acid molecules further comprises one or more barcode sequences unique to the synthase, the target enzyme or a combination thereof.


SEQUENCE LISTING (298 sequences)

SEQ ID NO: 1 - protein, 8 aa, synthetic construct
AVLQSGFR

SEQ ID NO: 2 - protein, 9 aa, synthetic construct
KARVLAEAM

SEQ ID NO: 3 - protein, 4 aa, synthetic construct
LRGG

SEQ ID NO: 4 - protein, 546 aa, synthetic construct
MALTEEKPIRPIANFPPSIWGDQFLIYEKQVEQGVEQIVNDLKKEVRQLLKEALDIPMKHANLLKLIDEIQRLGIPYHFEREIDHALQCIYETYGDNWNGDRSSLWFRLMRKQGYYVTCDVFNNYKDKNGAFKQSLANDVEGLLELYEATSMRVPGEIILEDALGFTRSRLSIMTKDAFSTNPALFTEIQRALKQPLWKRLPRIEAAQYIPFYQQQDSHNKTLLKLAKLEFNLLQSLHKEELSHVCKWWKAFDIKKNAPCLRDRIVECYFWGLGSGYEPQYSRARVFFTKAVAVITLIDDTYDAYGTYEELKIFTEAVERWSITCLDTLPEYMKPIYKLFMDTYTEMEEFLAKEGRTDLFNCGKEFVKEFVRNLMVEAKWANEGHIPTTEEHDPVVIITGGANLLTTTCYLGMSDIFTKESVEWAVSAPPLFRYSGILGRRLNDLMTHKAEQERKHSSSSLESYMKEYNVNEEYAQTLIYKEVEDVWKDINREYLTTKNIPRPLLMAVIYLCQFLEVQYAGKDNFTRMGDEYKHLIKSLLVYPMSI

SEQ ID NO: 5 - DNA, 966 bp, synthetic construct
  source 1..966: mol_type = other DNA
atggagatggaaaaggagttcgagcagatcgacaagtccgggagctgggcggccatttaccaggatatccgacatgaagccagtgacttcccatgtagagtggccaagcttcctaagaacaaaaaccgaaataggtacagagacgtcagtccctttgaccatagtcggattaaactacatcaagaagataatgactatatcaacgctagtttgataaaaatggaagaagcccaaaggagttacattcttacccagggccctttgcctaacacatgcggtcacttttgggagatggtgtgggagcagaaaagcaggggtgtcgtcatgctcaacagagtgatggagaaaggttcgttaaaatgcgcacaatactggccacaaaaagaagaaaaagagatgatctttgaagacacaaatttgaaattaacattgatctctgaagatatcaagtcatattatacagtgcgacagctagaattggaaaaccttacaacccaagaaactcgagagatcttacatttccactataccacatggcctgactttggagtccctgaatcaccagcctcattcttgaactttcttttcaaagtccgagagtcagggtcactcagcccggagcacgggcccgttgtggtgcactgcagtgcaggcatcggcaggtctggaaccttctgtctggctgatacctgcctcttgctgatggacaagaggaaagacccttcttccgttgatatcaagaaagtgctgttagaaatgaggaagtttcggatggggctgatccagacagccgaccagctgcgcttctcctacctggctgtgatcgaaggtgccaaattcatcatgggggactcttccgtgcaggatcagtggaaggagctttcccacgaggacctggagcccccacccgagcatatccccccacctccccggccacccaaacgaatcctggagccacacaattga

SEQ ID NO: 6 - protein, 321 aa, synthetic construct
MEMEKEFEQIDKSGSWAAIYQDIRHEASDFPCRVAKLPKNKNRNRYRDVSPFDHSRIKLHQEDNDYINASLIKMEEAQRSYILTQGPLPNTCGHFWEMVWEQKSRGVVMLNRVMEKGSLKCAQYWPQKEEKEMIFEDTNLKLTLISEDIKSYYTVRQLELENLTTQETREILHFHYTTWPDFGVPESPASFLNFLFKVRESGSLSPEHGPVVVHCSAGIGRSGTFCLADTCLLLMDKRKDPSSVDIKKVLLEMRKFRMGLIQTADQLRFSYLAVIEGAKFIMGDSSVQDQWKELSHEDLEPPPEHIPPPPRPPKRILEPHN

SEQ ID NO: 7 - protein, 593 aa, synthetic construct
MAQISESVSPSTDLKSTESSITSNRHGNMWEDDRIQSLNSPYGAPAYQERSEKLIEEIKLLFLSDMDDSCNDSDRDLIKRLEIVDTVECLGIDRHFQPEIKLALDYVYRCWNERGIGEGSRDSLKKDLNATALGFRALRLHRYNVSSGVLENFRDDNGQFFCGSTVEEEGAEAYNKHVRCMLSLSRASNILFPGEKVMEEAKAFTTNYLKKVLAGREATHVDESLLGEVKYALEFPWHCSVQRWEARSFIEIFGQIDSELKSNLSKKMLELAKLDFNILQCTHQKELQIISRWFADSSIASLNFYRKCYVEFYFWMAAAISEPEFSGSRVAFTKIAILMTMLDDLYDTHGTLDQLKIFTEGVRRWDVSLVEGLPDFMKIAFEFWLKTSNELIAEAVKAQGQDMAAYIRKNAWERYLEAYLQDAEWIATGHVPTFDEYLNNGTPNTGMCVLNLIPLLLMGEHLPIDILEQIFLPSRFHHLIELASRLVDDARDFQAEKDHGDLSCIECYLKDHPESTVEDALNHVNGLLGNCLLEMNWKFLKKQDSVPLSCKKYSFHVLARSIQFMYNQGDGFSISNKVIKDQVQKVLIVPVPI

SEQ ID NO: 8 - DNA, 1653 bp, synthetic construct
  source 1..1653: mol_type = other DNA
atggagttggtagacacccctagtcttgaggtattcgaggatgtagttgttgaccgtcaggtagctggcttcgatccgagtttctggggcgattattttattaccaaccagaagtcgcagtccgaagcgtggatgaacgaacgcgcagaagaattgaaaaacgaggtccgtagcatgttccagaacgtaactggaatcctgcaaacgatgaatcttatcgacacgattcaacttcttggacttgattatcacttcatggaagaaatcgcaaaggcccttgaccacttaaaggatgtcgatatgagtaagtatggcttatatgaagtggcccttcactttcgccttctgcgccagaagggctttaacatcagttctgacgtatttaaaaagtacaaagataaggaaggtaaatttatggaggaacttaaggatgacgcgaaagggttgttatcgttatacaatgcagcgtactttggtacgaaggaagaaacgattttggatgaagcaatttcttttactaaagacaacttaacatctttgttaaaggatttgaatccaccattcgcgaaacttgtatctctgacattaaagactccaattcaacgttcaatgaagcgcatctttacacgctcttacatctccatttaccaagacgagcccaccttgaacgaaactatcttagaattagccaagttggatttcaatatgttacaatgtttacaccagaaggagttgaagaaaatctgtgcctggtggaacaatctgaacttggatatcatgcacttgaatttcatccgcgaccgcgtggtagaatgttactgctggagtatggtcattcgtcacgaaccttcctgttctcgtgcccgcttaatcagcacgaaattgctgatgttaattactgtgcttgacgatacgtacgattcctatagtactctggaagagtcccgcctgcttacagacgccattcaacgttggaatcctaatgaggtggaccagttaccagaataccttcgcgacttctttcttaagatgttgaatatttttcaggaatttgagaacgaattagctccggaggagaaatttcgtattttatacttaaaggaagaatggaagatccagtcccaaagttacttcaaagaatgtcagtggcgtgatgataattatgttccaaagctggaagaacacatgcgtttatcgattattagtgtaggcttcgttttattctactgtggctttttatcaggtatggaggaggccgttgcaacgaaagacgcctttgaatggtttgcgtcctttccaaaaattattgaagcttgtgctacaattattcgtatcaccaatgatatcacgtccatggaacgtgaacaaaaacgcgcacatgtggcctcaactgtagactgttatatgaaggaatacggaacgtcaaaagacgtcgcgtgcgaaaaactgttgggcttcgtggaggacgcatggaagacgatcaatgaagagcttcttactgagactgggctttcacgcgaagtcattgaactttctttccattctgcacagactacggagttcgtatataagcacgttgatgcgttcaccgagccgaatactactatgaaagaaaacatcttctcgttattggtacatcccatccccatttaa

SEQ ID NO: 9 - protein, 550 aa, synthetic construct
MELVDTPSLEVFEDVVVDRQVAGFDPSFWGDYFITNQKSQSEAWMNERAEELKNEVRSMFQNVTGILQTMNLIDTIQLLGLDYHFMEEIAKALDHLKDVDMSKYGLYEVALHFRLLRQKGFNISSDVFKKYKDKEGKFMEELKDDAKGLLSLYNAAYFGTKEETILDEAISFTKDNLTSLLKDLNPPFAKLVSLTLKTPIQRSMKRIFTRSYISIYQDEPTLNETILELAKLDFNMLQCLHQKELKKICAWWNNLNLDIMHLNFIRDRVVECYCWSMVIRHEPSCSRARLISTKLLMLITVLDDTYDSYSTLEESRLLTDAIQRWNPNEVDQLPEYLRDFFLKMLNIFQEFENELAPEEKFRILYLKEEWKIQSQSYFKECQWRDDNYVPKLEEHMRLSIISVGFVLFYCGFLSGMEEAVATKDAFEWFASFPKIIEACATIIRITNDITSMEREQKRAHVASTVDCYMKEYGTSKDVACEKLLGFVEDAWKTINEELLTETGLSREVIELSFHSAQTTEFVYKHVDAFTEPNTTMKENIFSLLVHPIPI

SEQ ID NO: 10 - DNA, 1731 bp, synthetic construct
  source 1..1731: mol_type = other DNA
atggatgccttcgctacgtctcctacgtcagccttaatcaaagcagtaaactgcatcgcccacgtgacgcctatggctggcgaggattcctccgagaaccgtcgtgcgtcaaattacaaaccatcaacatgggactacgagttcttgcagagtttggctacctcgcataataccgtacaggaaaagcacatgaaaatggcggagaagttgaaagaagaagtcaagagtatgatcaaaggccagatggaacctgtggcgaagcttgaactgattaacatcgtccagcgtttgggtctgaaatatcgttttgaatctgaaattaaagaagagctgttctctttgtacaaggacggtaccgatgcatggtgggtcgataacttgcacgcaaccgcgctgcgttttcgtttactgcgcgagaacgggatcttcgtcccgcaggacgtgttcgaaacatttaaagacaagtccggaaagttcaagtcacaattgtgcaaggatgtgcgtgggttattgagtttgtatgaagcttcttacttaggctgggaaggcgaagatcttcttgacgaagctaagaagtttagcactacgaacttgaacaacgtgaaggaatcgatctcgtcaaatacgcttggccgtttagtcaaacatgccttaaatttgccgttacactggtctgctgcccgttacgaagcacgctggtttatcgacgaatacgaaaaggaagaaaacgtgaatcccaacttgcttaagtacgctaagttagatttcaacatcgtccaaagcatccatcagggtgagctgggcaatttagctcgttggtgggttgagacaggtctggacaagttgtctttcgtgcgtaatacgcttatgcagaactttatgtggggatgtgctatggtattcgaaccccagtacggcaaggtacgcgatgctgctgttaagcaggccagcttaattgcaatggtagatgacgtatatgacgtttacgggtcgctggaggaacttgagatctttaccgatatcgtcgatcgttgggacattaccggcattgacaaacttcctcgcaatatcagcatgattttattaacaatgttcaatacggcgaaccaaatcgggtatgatttgcttcgtgaccgtggttttaatggtatcccgcatattgcgcaagcttgggcaactctgtgtaaaaagtaccttaaagaagctaaatggtaccactccggatacaagccgaccttagaagagtacctggaaaacgggttggtgtcgatttcctttgtgctgtcactggtgactgcttatctgcagactgagacattggaaaatttgacctacgagtcggccgcgtatgttaactccgtacccccgttggtacgctatagtggattattaaatcgtttatataacgatctgggtacgtcatccgctgagattgcccgcggggacaccttaaagtctatccaatgctacatgactcagacgggggccacggaagaggcggcgcgcgaacacatcaaagggttagtacatgaggcatggaagggtatgaataagtgtcttttcgaacaaaccccatttgctgagccattcgtcggtttcaacgttaatacggtgcgcgggtcccagttcttttatcagcatggtgatgggtacgccgtcacagaaagttggaccaaagacctgtccctttcagttttaattcatccaattcccttgaatgaggaggactaa

SEQ ID NO: 11 - protein, 576 aa, synthetic construct
MDAFATSPTSALIKAVNCIAHVTPMAGEDSSENRRASNYKPSTWDYEFLQSLATSHNTVQEKHMKMAEKLKEEVKSMIKGQMEPVAKLELINIVQRLGLKYRFESEIKEELFSLYKDGTDAWWVDNLHATALRFRLLRENGIFVPQDVFETFKDKSGKFKSQLCKDVRGLLSLYEASYLGWEGEDLLDEAKKFSTTNLNNVKESISSNTLGRLVKHALNLPLHWSAARYEARWFIDEYEKEENVNPNLLKYAKLDFNIVQSIHQGELGNLARWWVETGLDKLSFVRNTLMQNFMWGCAMVFEPQYGKVRDAAVKQASLIAMVDDVYDVYGSLEELEIFTDIVDRWDITGIDKLPRNISMILLTMFNTANQIGYDLLRDRGFNGIPHIAQAWATLCKKYLKEAKWYHSGYKPTLEEYLENGLVSISFVLSLVTAYLQTETLENLTYESAAYVNSVPPLVRYSGLLNRLYNDLGTSSAEIARGDTLKSIQCYMTQTGATEEAAREHIKGLVHEAWKGMNKCLFEQTPFAEPFVGFNVNTVRGSQFFYQHGDGYAVTESWTKDLSLSVLIHPIPLNEED

SEQ ID NO: 12 - DNA, 2409 bp, synthetic construct
  source 1..2409: mol_type = other DNA
atgagcagcagcactggcactagcaaggtggtttccgagacttccagtaccattgtggatgatatccctcgactctccgccaattatcatggcgatctgtggcaccacaatgttatacaaactctggagacaccgtttcgtgagagttctacttaccaagaacgggcagatgagctggttgtgaaaattaaagatatgttcaatgcgctcggagacggagatatcagtccgtctgcatacgacactgcgtgggtggcgaggctggcgaccatttcctctgatggatctgagaagccacggtttcctcaggccctcaactgggttttcaacaaccagctccaggatggatcgtggggtatcgaatcgcactttagtttatgcgatcgattgcttaacacgaccaattctgttatcgccctctcggtttggaaaacagggcacagccaagtacaacaaggtgctgagtttattgcagagaatctaagattactcaatgaggaagatgagttgtccccggatttccaaataatctttcctgctctgctgcaaaaggcaaaagcgttggggatcaatcttccttacgatcttccatttatcaaatatttgtcgacaacacgggaagccaggcttacagatgtttctgcggcagcagacaatattccagccaacatgttgaatgcgttggaaggactcgaggaagttattgactggaacaagattatgaggtttcaaagtaaagatggatctttcctgagctcccctgcctccactgcctgtgtactgatgaatacaggggacgaaaaatgtttcacttttctcaacaatctgctcgacaaattcggcggctgcgtgccctgtatgtattccatcgatctgctggaacgcctttcgctggttgataacattgagcatctcggaatcggtcgccatttcaaacaagaaatcaaaggagctcttgattatgtctacagacattggagtgaaaggggcatcggttggggcagagacagccttgttccagatctcaacaccacagccctcggcctgcgaactcttcgcatgcacggatacaatgtttcttcagacgttttgaataatttcaaagatgaaaacgggcggttcttctcctctgcgggccaaacccatgtcgaattgagaagcgtggtgaatcttttcagagcttccgaccttgcatttcctgacgaaagagctatggacgatgctagaaaatttgcagaaccatatcttagagaggcacttgcaacgaaaatctcaaccaatacaaaactattcaaagagattgagtacgtggtggagtacccttggcacatgagtatcccacgcttagaagccagaagttatattgattcatatgacgacaattatgtatggcagaggaagactctatatagaatgccatctttgagtaattcaaaatgtttagaattggcaaaattggacttcaatatcgtacaatctttgcatcaagaggagttgaagcttctaacaagatggtggaaggaatccggcatggcagatataaatttcactcgacaccgagtggcggaggtttatttttcatcagctacatttgaacccgaatattctgccactagaattgccttcacaaaaattggttgtttacaagtcctttttgatgatatggctgacatctttgcaacactagatgaattgaaaagtttcactgagggagtaaagagatgggatacatctttgctacatgagattccagagtgtatgcaaacttgctttaaagtttggttcaaattaatggaagaagtaaataatgatgtggttaaggtacaaggacgtgacatgctcgctcacataagaaaaccctgggagttgtacttcaattgttatgtacaagaaagggagtggcttgaagccgggtatataccaacttttgaagagtacttaaagacttatgctatatcagtaggccttggaccgtgtaccctacaaccaatactactaatgggtgagcttgtgaaagatgatgttgttgagaaagtgcactatccctcaaatatgtttgagcttgtatccttgagctggcgactaacaaacgacaccaaaacatatcaggctgaaaaggctcgaggacaacaagcctcaggcatagcatgctatatgaaggataatccaggagcaactgaggaagatgccattaagcacatatgtcgtgttgttgatcgggccttgaaagaagcaagctttgaatatttcaaaccatccaatgatatcccaatgggttgcaagtcctttatttttaaccttagattgtgtgtccaaatcttttacaagtttatagatgggtacggaatcgccaatgaggagattaaggactatataagaaaagtttatattgatccaattcaagtatga

SEQ ID NO: 13 - protein, 862 aa, synthetic construct
MAQLSFNAALKMNALGNKAIHDPTNCRAKSERQMMWVCSRSGRTRVKMSRGSGGPGPVVMMSSSTGTSKVVSETSSTIVDDIPRLSANYHGDLWHHNVIQTLETPFRESSTYQERADELVVKIKDMFNALGDGDISPSAYDTAWVARLATISSDGSEKPRFPQALNWVFNNQLQDGSWGIESHFSLCDRLLNTTNSVIALSVWKTGHSQVQQGAEFIAENLRLLNEEDELSPDFQIIFPALLQKAKALGINLPYDLPFIKYLSTTREARLTDVSAAADNIPANMLNALEGLEEVIDWNKIMRFQSKDGSFLSSPASTACVLMNTGDEKCFTFLNNLLDKFGGCVPCMYSIDLLERLSLVDNIEHLGIGRHFKQEIKGALDYVYRHWSERGIGWGRDSLVPDLNTTALGLRTLRMHGYNVSSDVLNNFKDENGRFFSSAGQTHVELRSVVNLFRASDLAFPDERAMDDARKFAEPYLREALATKISTNTKLFKEIEYVVEYPWHMSIPRLEARSYIDSYDDNYVWQRKTLYRMPSLSNSKCLELAKLDFNIVQSLHQEELKLLTRWWKESGMADINFTRHRVAEVYFSSATFEPEYSATRIAFTKIGCLQVLFDDMADIFATLDELKSFTEGVKRWDTSLLHEIPECMQTCFKVWFKLMEEVNNDVVKVQGRDMLAHIRKPWELYFNCYVQEREWLEAGYIPTFEEYLKTYAISVGLGPCTLQPILLMGELVKDDVVEKVHYPSNMFELVSLSWRLTNDTKTYQAEKARGQQASGIACYMKDNPGATEEDAIKHICRVVDRALKEASFEYFKPSNDIPMGCKSFIFNLRLCVQIFYKFIDGYGIANEEIKDYIRKVYIDPIQV

SEQ ID NO: 14 - DNA, 1710 bp, synthetic construct
  source 1..1710: mol_type = other DNA
atgagcaacttcctggtgagcacttgtagtagcccattagcgctggatgaaaacaattcgacgaaacaggaccatgtgatccgtaataccgttacgtttcatagttctatatggggtgatcaatttcttacctatgatgagaaagacgatctggtggcggaaaagcaattggccgaagagctgatcgaagagacacgcaaggaattaattatcaccacctctagccatgaaccaatccaacatatgaaacttatccagctgatcgatgctgtccagcgattaggagttgcctatcactttgagaaagagatagaagatgcccttcaacacgtataccgtacatatggccaccagggtatacacaacaacaatgacctccagtccatttcactctggttccgcatactgagacaacagggtttcaatgtgagcagcgaaatctttaagaaccatatggatgaaaaaggcaacctgtttagcaatgatgtgcagtcgatgctggcactctatgaggcctcctatatgcgtgtggagggcgagaaggtgcttgatgatgcactggagtttaccaagacacacctcgcaattattgcaaaagacccgtcatgtgattcttcattgcggacccagattcaagacgcgctgagacagccactgagaaaacgactcccgcgtttggaggccgtgcgctacattccgatatatcagcagcagagtagccataatcaaattctgctgaaattggccaaattagatttcaacatgctgcagacgatgcacaaaaaggaacttagcgagatttgtaaatggtggaaagatctcgatatgcagaacaaactgccgtttgttcgcgatcgtttgattgaaggctacttttggatactgggtatctatttcgaacctcatcattcccgatcacgtatgtttctcataaagagctgtatgtggcttgtagttatcgatgacacctttgacaattacggcacctatgaagagctcgaaatatttactgaagccgttgagcgttggtcgattaattgcctggatatgttgcctgagtatatgaaactgatttacaaagagcttgtgattgttcaccaggaaatggaggaaacgttagaaaaagaagggaaagcttatcacattcatcatgtgaaggagctcgctaaggagtgcacccgaagcttgctcgtcgaagcaaaatggcttaaagaagggtatatgccgacattggacgagtatattagtaattctttgattacgtgtgcttatgcggttatgattgcccggagctacgtaggcggtgacgataaattagttaatgaagactccttcaagtgggttgcgacccacccgccactggtgaaagcgtcctgtttgattcttcgtctgatggatgatattgcgacccataaagaggaacaggagcgtggccatgtggcaagcagtattgaatgctatattaaagagacaggtgctacagaggaagaggcccgtgaacactttagtaaacaggtggaagatgcctggaaagttgttaatcgtgagagccttcgtccgaccgcggttgcctttccgctcgtcatgccggcgataaatctcgcccgcatgtgcgatgcgttatataagggtaaccatgatggatataatcacgcgggtaaggaagtgatccagtatataaaatctctgctcgtacatcctttgatctaa

SEQ ID NO: 15 - protein, 567 aa, synthetic construct
MSTSSVSISLSSLVIDENNSTKQDHVIRNTVTFHPSIWGDQFLVYDEKDDLVAEKQLVEELTEEIRKKLFITASSIHEPLQQIQLIDAIQRLGVAYHFEKEIEEALQHVYRTYGHQGIHNNNNLQSVSLWFRILRQQGFNVSPEIFKNHMDEKGNLLSNDVESMLALYEASYMRVEGEKVLDDALKFTKTHLAIIAQHPSCDSSLRTQIQEALRQPLRKRLPRLEAVRYIPIYQQQSSHNQLLLKLAKLDFNMLQSMHKKELSQICKWWKDLDMQNKLPFVRDRLIEGYFCILGIYFEPHHSRLRMFLIKSCMWLIVMDDTFDNYGTYEELKIFTEVVERWSISCLDLLPEYMKVIYLELVNIHQEMEESLEKEGKTYHIYYVKEMAKEYTRSLLAEAKWLKDGYMPTLDEYISNSLITTTYAVVIEGSYVGGPDMLVTEDSFKWVATHPPLVKASCLILRLMNDIATHKEEQERSHVASSIECYIKETGATEEEACEYFSKQVEDAWKVINRDSLKPTDVPFPLVKPVINLARISDVVYKGSINGYNHAGKELIQNIKSLLVHPLI

SEQ ID NO: 16 - DNA, 1641 bp, synthetic construct
  source 1..1641: mol_type = other DNA
atgtcacttactgaggaaaagcccattcgcccaatcgcgaattttagccccagtatttgggaagaccagttcttgatctatgctaagcaggttgagcatggcgtggagcagcgtgttaaggacctcacaaaggaagtgcgtcagctgctcaaagaggcgctcgacatcccgatgaagcacgcgaatttattgaagttgatcgacgagatccagcgtttgggcatcagctacttatttgaacaagaaatcgaccatgctctccagcatatttacgagacctacggcgacaactggtcgggtgatcgcagctcactgtggttccgtctgatgcgtaagcagggctactttgtaacgtgtgacgtcttcaacaaccacaaggatgagagcggagcgttcaagcaaagccttgccaacgacgtcgaaggtttacttgagttgtatgaggcaacctctatgcgtgtagcaggagaaatcatccttgacgacgctttggttttcactcgcagtaacttgtctattatcgcgaaagacacgcttagtactaatcccgctttgagtaccgagattcagcgcgcattgaaacaaccgctttggaagcgtctcccgcgcattgaagctgctcaatacattcctttctatgaacaacaggatagccataatatggcgctgcttaagttggcaaagttagaattcaaccttctgcaatcgctgcatcgcgaagagctttcccaattgtctaagtggtggaaagctttcgatgtaaagaacaatgcaccgtacagtcgcgatcgcatcgtggagtgttatttctggggcttagctagccgttttgaaccccaattctctcgcgcccgtatcttccttgcaaaagtaattgccttggtcacgttaattgacgatacctatgacgcatatggcacgtatgaagaactcaagattttcactgaagcgatcgagcgctggagcatcacatgtttggatatgatccctgagtacatgaagcctatttacaagctgttaatggacacttatacggagatggaagaggttcttgcgaaggaagggaagacggatatctttgactgcggcaaggagtttgtgaaggactttgtccgtgtacttatggtagaggcccagtggcttaacgaaggccacattcccacgaccgaggaattagattctatcgcggtgaacctcggtggcgcgaatttattaacgactacctgctatctgggtatgtctgacatcgtcacaaaagaagcggtcgaatgggctgtgagtgagccgcctctgttacgttataagggtattcttggtcgtcgtttaaatgaccttgccgggcacaaggaagagcaggaacgtaaacacgtgtcgtcgtctgtcgagagttacatgaaggagtataatgtgtccgaggaatacgctcaaaatctgctttataaacaggttgaggatctgtggaaggacattaaccgtgagtatttgatcactaagacaatcccccgtcctttactggtagcggtgatcaatctcgtgcactttctggaagtcctgtacgcggagaaggacaactttactcgcatgggtgacgagtataaggacctggtgaagtcattactcgtctaccctatgagtatctga

SEQ ID NO: 17 - protein, 546 aa, synthetic construct
MSLTEEKPIRPIANFSPSIWEDQFLIYAKQVEHGVEQRVKDLTKEVRQLLKEALDIPMKHANLLKLIDEIQRLGISYLFEQEIDHALQHIYETYGDNWSGDRSSLWFRLMRKQGYFVTCDVFNNHKDESGAFKQSLANDVEGLLELYEATSMRVAGEIILDDALVFTRSNLSIIAKDTLSTNPALSTEIQRALKQPLWKRLPRIEAAQYIPFYEQQDSHNMALLKLAKLEFNLLQSLHREELSQLSKWWKAFDVKNNAPYSRDRIVECYFWGLASRFEPQFSRARIFLAKVIALVTLIDDTYDAYGTYEELKIFTEAIERWSITCLDMIPEYMKPIYKLLMDTYTEMEEVLAKEGKTDIFDCGKEFVKDFVRVLMVEAQWLNEGHIPTTEELDSIAVNLGGANLLTTTCYLGMSDIVTKEAVEWAVSEPPLLRYKGILGRRLNDLAGHKEEQERKHVSSSVESYMKEYNVSEEYAQNLLYKQVEDLWKDINREYLITKTIPRPLLVAVINLVHFLEVLYAEKDNFTRMGDEYKDLVKSLLVYPMSI

SEQ ID NO: 18 - DNA, 1641 bp, synthetic construct
  source 1..1641: mol_type = other DNA
atgaatagtaccagccgtcgttcagcaaactataaaccgaccatttggaataatgaatacctgcagtcactgaacagcatctatggagaaaagagattccttgaacaggccgaaaaactgaaagatgaagttcgcatgctgctggaaaaaacctctgacccgctggaccacatcgaacttgttgacgtcctgcaacgccttgcaatatcgtaccattttactgaatatatcgatcggaatttgaaaaatatttacgatatactcatcgacgggcggcggtggaatcacgcggataacctgcatgccacgactctctcctttagacttttacgtcagcatggttaccaagtttcgccagaagtctttcggaatttcatggatgaaaccggaaatttcaaaaaaaacctgtgcgatgacataaaaggactccttagcttgtatgaagcgagctatttgctcacggaaggtgaaaccataatggattcagcccaggcgtttgctacccatcaccttaaacagaaactggaggaaaatatgaataaaaatcttggagatgagatagcccatgcgctggaattgccgctgcactggagagtccccaagctggacgtaagatggtccattgacgcttatgagcgtcgacaggatatgaatccacttcttttggagctggccaaactggattttaatatcgcccagagtatgtaccaagatgaattaaaagaattaagtcgttggtattcaaaaacacacctgccggaaaaattggcgttcgcacgtgatcgccttgtggaatcctacttgtggggacttggattagcatcagaaccccatcataaatattgtcgcatgatggtggcccagagtactaccctgattagcatcatcgatgatatatatgatgtttatggtacgctggatgaattgcagctgtttacgcatgcagtcgaccgttgggacattaaatatctggaacaattacccgaatacatgcagatttgtttcttagcgttgtttaatactgtgaacgagcgttcatatgactttttactcgacaaaggttttaacgttatcccgcactcgagttatcggtgggcagagctgtgcaagacttacctgatagaggcgaattggtaccactctggctataaacccagcttaaatgaatatcttaaccaggggctgatctcagtcgcaggcccacatgccttgtcacacacttatctgtgcatgactgatagtcttaaggaaaaacatatactcgacctgcgcacaaatcctcctgtgatcaaatgggttagtatccttgtaagactggcagacgatctcggtacttctacggacgaattgaaacgtggggataatccaaagtcaatccagtgccatatgcatgacactggctgtaatgaagaggagacacgcgcctacatcaaaaatttaattggttccacctggaaaaagattaataaagatgttctcatgaattttgagtattcgatggattttcggacagcggcgatgaatggtgcgcgcgtaagccagtttatgtatcagtacgatgatgatggacacggggtgcctgagggcaagtcgaaagaacgtgtttgttccctgatcgtcgaacctattccactgccttag

SEQ ID NO: 19 - protein, 546 aa, synthetic construct
MNSTSRRSANYKPTIWNNEYLQSLNSIYGEKRFLEQAEKLKDEVRMLLEKTSDPLDHIELVDVLQRLAISYHFTEYIDRNLKNIYDILIDGRRWNHADNLHATTLSFRLLRQHGYQVSPEVFRNFMDETGNFKKNLCDDIKGLLSLYEASYLLTEGETIMDSAQAFATHHLKQKLEENMNKNLGDEIAHALELPLHWRVPKLDVRWSIDAYERRQDMNPLLLELAKLDFNIAQSMYQDELKELSRWYSKTHLPEKLAFARDRLVESYLWGLGLASEPHHKYCRMMVAQSTTLISIIDDIYDVYGTLDELQLFTHAVDRWDIKYLEQLPEYMQICFLALFNTVNERSYDFLLDKGFNVIPHSSYRWAELCKTYLIEANWYHSGYKPSLNEYLNQGLISVAGPHALSHTYLCMTDSLKEKHILDLRTNPPVIKWVSILVRLADDLGTSTDELKRGDNPKSIQCHMHDTGCNEEETRAYIKNLIGSTWKKINKDVLMNFEYSMDFRTAAMNGARVSQFMYQYDDDGHGVPEGKSKERVCSLIVEPIPLP

SEQ ID NO: 20 - DNA, 1782 bp, synthetic construct
  source 1..1782: mol_type = other DNA
atggctcaaatcagcgaatcagtgtctccaagcaccgaccttaaaagcacggaatcttctattaccagcaaccgccacggtaacatgtgggaagatgaccgcattcagagcttaaacagcccatatggcgcacccgcttatcaggaacgtagcgaaaaattgattgaagaaattaagctcctgtttctgtccgatatggacgatagttgcaatgattcggatcgcgacttgatcaaacgcctggagatcgtagatacggttgagtgtctgggcattgatcgtcatttccaacctgaaattaagctggcgctggattacgtgtaccgttgctggaatgagcgtggcatcggagaaggtagccgtgatagcttaaaaaaggacctgaatgcgaccgccttgggctttcgggctttacgcttacaccgttataatgtaagctcaggagtgctggagaacttccgtgatgacaatggtcaattcttttgcggttctactgtggaggaggaaggcgcggaggcctacaataaacatgtacgttgcatgctgtccctgtcccgcgcttccaatattttattcccgggcgagaaagtgatggaagaagcgaaggcgtttacgaccaactatcttaagaaagtcctggcgggtcgtgaagcaactcatgtcgacgagagtctccttggagaggtcaagtatgcactagaatttccgtggcattgttccgtgcagcgctgggaggcacgttcttttatcgaaattttcggtcagattgatagtgaactgaaaagcaacctctctaaaaaaatgctcgaactcgcaaaacttgattttaacatactccagtgtacgcatcaaaaagagctccagatcattagtcgatggttcgccgattcaagtatcgcaagtctgaacttttaccgtaaatgctatgtggaattttacttctggatggccgcggcaatttcagaaccagaatttagtggctctcgcgtggcattcactaaaattgcgatcttgatgacaatgttagatgacttatacgacacgcatgggacgctggatcaattgaaaatatttaccgaaggtgtgcgcaggtgggacgtgtcgctggtggagggcctgccggatttcatgaaaattgcctttgagttctggttaaagacctccaacgaactgattgcggaggcggttaaggcccaaggccaggatatggcggcctatatccgcaaaaacgcttgggaacgctatctggaagcgtatttgcaggatgccgaatggatcgccaccggtcacgttccgacattcgatgaatatctgaacaatggcacccccaacaccggtatgtgtgtacttaatctgatcccgttgctgcttatgggcgaacacttgccgatcgatattcttgaacagatctttctgccgagccggttccaccatctgattgaactggctagccgactggtcgatgatgcgagagattttcaagccgaaaaagatcatggtgatttatcctgcatcgaatgctacctgaaagaccatccggaatcaacagttgaagacgccctgaatcacgtcaacggcctgctggggaattgtttgctggaaatgaattggaaatttctgaaaaaacaggactcggtacctctgtcgtgtaaaaaatactcattccacgtcctggcgcggtcgattcagtttatgtataaccagggggacgggttttcgatttcgaacaaagttattaaagaccaggtccagaaagttctaatcgttccggttcctatataa

SEQ ID NO: 21 - skipped

SEQ ID NO: 22 - DNA, 1665 bp, synthetic construct
  source 1..1665: mol_type = other DNA
atgaaccagctggcaatggttaatacaactatcacccgcccattagctaattaccattcgtccgtctggggtaactatttcctcagttatactcctcagctgacagaaattagttcacaggagaagcgtgaacttgaagaactgaaggaaaaagttcggcaaatgctggtagaaaccccagataattcgactcaaaaattagtcttaatcgatacgattcaacgtctgggcgtagcatatcattttgaaaaccatatcaaaataagtattcagaatattttcgatgagtttgaaaaaaataaaaataaagataatgatgatgacttgtgtgttgtcgctcttcgttttagactggtccgggggcagcgtcattacatgtccagcgatgtctttactcggttcacaaatgatgacggtaaatttaaagagactctgaccaaagacgttcagggcttgctgaacctgtatgaagcgacccatcttcgggtccatggcgaggaaatcctggaggaagcgttgtcctttactgtaacgcacctgaagtcgatgtcgcctaagctggataactcactcaaagctcaggttagtgaggcacttttccagccgatacacactaacatcccacgggtagtcgcacgtaaatatattcgtatttacgagaatattgagagtcatgatgatttactgctgaagtttgcaaagctggatttccatattcttcaaaaaatgcatcagcgtgagctgtccgaactgacaagatggtggaaagatctggatcattcgaataaatatccgtatgcacgcgacaagctggtggaatgttatttttgggctattggcgtatactttggcccccagtataagcgtgcgcgtcgaacgctgacaaagctgattgttatcataaccatcactgatgacttatatgatgcttacgcgacgtacgatgaattggtgccctatacaaacgcggtagaacgatgtgaaatatcggcgatgcactcgatttctccatatatgcgccccttgtatcaagtgtttctggattatttcgatgaaatggaagaggagttaactaaagatggcaaagcgcattatgtgtattatgctaagatcgaaacgaacaaatggatcaaatcctatttgaaggaagcggaatggctgaaaaatgacattatcccgaaatgtgaagaatataaacgtaatgctacaattacgatttctaatcagatgaacctgattacgtgcttgattgtcgcaggtgaattcataagcaaagagacctttgaatggatgattaacgagagtctgattgcgcccgcatctagcctcattaaccgtctcaaggatgatattataggtcacgagcatgagcaacagcgtgagcacggcgcaagttttattgagtgctacgtcaaagagtatcgtgcatccaagcaggaggcgtacgtagaggcacgccgtcagatcacaaatgcatggaaggatataaacacagactaccttcatgcgactcaagttccgaccttcgtacttgagcccgctctgaacctcagccgtttggtagatatcctgcaggaggacgattttaccgattctcagaattttctgaaagataccattacactgctgttcgtggacagtgtaaactctacatcatgcggctaa

SEQ ID NO: 23 - protein, 554 aa, synthetic construct
MNQLAMVNTTITRPLANYHSSVWGNYFLSYTPQLTEISSQEKRELEELKEKVRQMLVETPDNSTQKLVLIDTIQRLGVAYHFENHIKISIQNIFDEFEKNKNKDNDDDLCVVALRFRLVRGQRHYMSSDVFTRFTNDDGKFKETLTKDVQGLLNLYEATHLRVHGEEILEEALSFTVTHLKSMSPKLDNSLKAQVSEALFQPIHTNIPRVVARKYIRIYENIESHDDLLLKFAKLDFHILQKMHQRELSELTRWWKDLDHSNKYPYARDKLVECYFWAIGVYFGPQYKRARRTLTKLIVIITITDDLYDAYATYDELVPYTNAVERCEISAMHSISPYMRPLYQVFLDYFDEMEEELTKDGKAHYVYYAKIETNKWIKSYLKEAEWLKNDIIPKCEEYKRNATITISNQMNLITCLIVAGEFISKETFEWMINESLIAPASSLINRLKDDIIGHEHEQQREHGASFIECYVKEYRASKQEAYVEARRQITNAWKDINTDYLHATQVPTFVLEPALNLSRLVDILQEDDFTDSQNFLKDTITLLFVDSVNSTSCG

SEQ ID NO: 24 - DNA, 225 bp, synthetic construct
  source 1..225: mol_type = other DNA
caaatctttgtcaagacattaacaggtaagaccatcacgttggaggtagaatcgagtgatactatcgacaatgtaaaagcaaaaatccaagacaaggagggcattcccccagaccagcaacgcttgatttttgcgggaaagcaacttgaggatggccgtactttagcggactataatatccagaaagaatctacattgcacttagtgttgcgcctgcgtgggggc

SEQ ID NO: 25 - protein, 75 aa, synthetic construct
QIFVKTLTGKTITLEVESSDTIDNVKAKIQDKEGIPPDQQRLIFAGKQLEDGRTLADYNIQKESTLHLVLRLRGG

SEQ ID NO: 26 - protein, 12 aa, synthetic construct
KARVLAEAMAAA

SEQ ID NO: 27 - protein, 19 aa, synthetic construct
AAAAAVLQSGFRAAAAAAA

SEQ ID NO: 28 - skipped

SEQ ID NO: 29 - protein, 6 aa, synthetic construct
AAELQA

SEQ ID NO: 30 - protein, 16 aa, synthetic construct
AAKARVLAEAMAAAAA

SEQ ID NO: 31 - protein, 20 aa, synthetic construct
AAAAKARVLAEAMAAAAAAA

SEQ ID NO: 32 - protein, 11 aa, synthetic construct
AVLQSGFRAAA

SEQ ID NO: 33 - protein, 15 aa, synthetic construct
AAAVLQSGFRAAAAA

SEQ ID NO: 34 - protein, 707 aa, synthetic construct
MKFGNFLLTYQPPQFSQTEVMKRLVKLGRISEECGFDTVWLLEHHFTEFGLLGNPYVAAAYLLGATKKLNVGTAAIVLPTAHPVRQLEDVNLLDQMSKGRFRFGICRGLYNKDFRVFGTDMNNSRALAECWYGLIKNGMTEGYMEADNEHIKFHKVKVNPAAYSRGGAPVYVVAESASTTEWAAQFGLPMILSWIINTNEKKAQLELYNEVAQEYGHDIHNIDHCLSYITSVDHDSIKAKEICRKFLGHWYDSYVNATTIFDDSDQTRGYDFNKGQWRDFVLKGHKDTNRRIDYSYEINPVGTPQECIDIIQKDIDATGISNICCGFEANGTVDEIIASMKLFQSDVMPFLKEKQRSLLYYGGGGSGGGGSGGGGSGGGGSKFGLFFLNFINSTTVQEQSIVRMQEITEYVDKLNFEQILVYENHFSDNGVVGAPLTVSGFLLGLTEKIKIGSLNHIITTHHPVRIAEEACLLDQLSEGRFILGFSDCEKKDEMHFFNRPVEYQQQLFEECYEIINDALTTGYCNPDNDFYSFPKISVNPHAYTPGGPRKYVTATSHHIVEWAAKKGIPLIFKWDDSNDVRYEYAERYKAVADKYDVDLSEIDHQLMILVNYNEDSNKAKQETRAFISDYVLEMHPNENFENKLEEIIAENAVGNYTECITAAKLAIEKCGAKSVLLSFEPMNDLMSQKNVINIVDDNIKKYHTEYT

SEQ ID NO: 35 - protein, 10 aa, synthetic construct
KARVLXAEAM

SEQ ID NO: 36 - protein, 9 aa, synthetic construct
AVLQXSGFR

SEQ ID NO: 37 - protein, 5 aa, synthetic construct
LRGGX

SEQ ID NO: 38 - protein, 27 aa, synthetic construct
ATKTGCSAMCMTTAAAGGAGSTTCCAA

SEQ ID NO: 39 - DNA, 27 bp, synthetic construct
  source 1..27: mol_type = other DNA
atttgccaccattaaaggagcttccaa

SEQ ID NO: 40 - DNA, 26 bp, synthetic construct
  source 1..26: mol_type = other DNA
tgtgccaaccttaaaggaggttccaa

SEQ ID NO: 41 - DNA, 27 bp, synthetic construct
  source 1..27: mol_type = other DNA
atttgccaacattaaaggaggttccaa

SEQ ID NO: 42 - DNA, 26 bp, synthetic construct
  source 1..26: mol_type = other DNA
tgtgccaacattaaaggagcttccaa

SEQ ID NO: 43 - DNA, 450 bp, synthetic construct
  source 1..450: mol_type = other DNA
ggggagtttctggcaaccacactggaacggatcgagaaaaatttcgtgattactgatccgagactgcctgacaacccaatcatttttgcgagcgattccttcctgcagctgacagaatattctcgggaagagatcctggggcgcaattgccgttttctgcagggacccgagacagaccgtgccactgttcggaaaatcagagatgctattgacaaccagactgaagtgaccgttcagctgatcaattataccaagagcggcaagaagttctggaacgtgttccacctgcagccgatgcgcgattataagggcgacgtccagtacttcattggcgtgcagctggatggcaccgaacgtcttcatggcgccgctgagcgtgaggcggtctgcctgatcaaaaagacagcctttcagattgctgaggcagcgaacgacgaaaattacttttaa

SEQ ID NO: 44 - protein, 149 aa, synthetic construct
GEFLATTLERIEKNFVITDPRLPDNPIIFASDSFLQLTEYSREEILGRNCRFLQGPETDRATVRKIRDAIDNQTEVTVQLINYTKSGKKFWNVFHLQPMRDYKGDVQYFIGVQLDGTERLHGAAEREAVCLIKKTAFQIAEAANDENYF

SEQ ID NO: 45 - DNA, 27 bp, synthetic construct
  source 1..27: mol_type = other DNA
gaggcagcgaacgacgaaaattacttt

SEQ ID NO: 46 - protein, 9 aa, synthetic construct
EAANDENYF

SEQ ID NO: 47 - DNA, 339 bp, synthetic construct
  source 1..339: mol_type = other DNA
tccagctccccgaaacgccctaagctgctgcgtgaatattacgattggctggttgataacagctttaccccatatctggtggtggatgccacatacctgggcgtgaacgtgcccgtggagtatgtgaaagacggtcagatcgtgctgaatctgtctgcaagtgcgaccggcaacctgcaactgacaaatgattttatccagttcaacgcccgctttaagggcgtgtctcgtgaactgtatatcccgatgggtgccgctctggccatttacgctcgcgagaacggcgatggtgtgatgttcgaaccagaagaaatctatgacgagctgaatattggttaa

SEQ ID NO: 48 - protein, 112 aa, synthetic construct
SSSPKRPKLLREYYDWLVDNSFTPYLVVDATYLGVNVPVEYVKDGQIVLNLSASATGNLQLTNDFIQFNARFKGVSRELYIPMGAALAIYARENGDGVMFEPEEIYDELNIG

SEQ ID NO: 49 - protein, 13 aa, synthetic construct
AAAVLQXSGFRAA

SEQ ID NO: 50 - protein, 17 aa, synthetic construct
AAAAAVLQXSGFRAAAA

SEQ ID NO: 51 - protein, 8 aa, synthetic construct
GLKRGGAK

SEQ ID NO: 52 - protein, 9 aa, synthetic construct
GLKRXGGAK

SEQ ID NO: 53 - DNA, 225 bp, synthetic construct; identical to SEQ ID NO: 24
  source 1..225: mol_type = other DNA

SEQ ID NO: 54 - protein, 83 aa, synthetic construct
AAAAQIFVKTLTGKTITLEVESSDTIDNVKAKIQDKEGIPPDQQRLIFAGKQLEDGRTLADYNIQKESTLHLVLRLRGGAAAA

SEQ ID NO: 55 - protein, 361 aa, Abies grandis
MHAFPHGTTATPTAIAVPPSLRLPVIEAAFPRQLHPYWPKLQETTRTWLLEKRLMPADKVEEYADGLCYTDLMAGYYLGAPDEVLQAIADYSAWFFVWDDRHDRDIVHGRAGAWRRLRGLLHTALDSPGDHLHHEDTLVAGFADSVRRLYAFLPATWNARFARHFHTVIEAYDREFHNRTRGIVPGVEEYLELRRLTFAHWIWTDLLEPSSGCELPDAVRKHPAYRRAALLSQEFAAWYNDLCSLPKEIAGDEVHNLGISLITHHSLTLEEAIGEVRRRVEECITEFLAVERDALRFADELADGTVRGKELSGAVRANVGNMRNWFSSVYWFHHESGRYMVDSWDDRSTPPYVNNEAAGEK

SEQ ID NO: 56 - protein, 581 aa, Abies grandis
MAEISESSIPRRTGNHHGNVWDDDLIHSLNSPYGAPAYYELLQKLIQEIKHLLLTEMEMDDGDHDLIKRLQIVDTLECLGIDRHFEHEIQTAALDYVYRWWNEKGIGEGSRDSFSKDLNATALGFRALRLHRYNVSSGVLKNFKDENGKFFCNFTGEEGRGDKQVRSMLSLLRASEISFPGEKVMEEAKAFTREYLNQVLAGHGDVTDVDQSLLREVKYALEFPWHCSVPRWEARSFLEIYGHNHSWLKSNINQKMLKLAKLDFNILQCKHHKEIQFITRWWRDSGISQLNFYRKRHVEYYSWVVMCIFEPEFSESRIAFAKTAILCTVLDDLYDTHATLHEIKIMTEGVRRWDLSLTDDLPDYIKIAFQFFFNTVNELIVEIVKRQGRDMTTIVKDCWKRYIESYLQEAEWIATGHIPTFNEYIKNGMASSGMCILNLNPLLLLDKLLPDNILEQIHSPSKILDLLELTGRIADDLKDFEDEKERGEMASSLQCYMKENPESTVENALNHIKGILNRSLEEFNWEFMKQDSVPMCCKKFTFNIGRGLQFIYKYRDGLYISDKEVKDQIFKILVHQVPMEE

SEQ ID NO: 57 - protein, 593 aa, Abies grandis; identical to SEQ ID NO: 7

SEQ ID NO: 58 - protein, 868 aa, Abies grandis
MAMPSSSLSSQIPTAAHHLTANAQSIPHFSTTLNAGSSASKRRSLYLRWGKGSNKIIACVGEGGATSVPYQSAEKNDSLSSSTLVKREFPPGFWKDDLIDSLTSSHKVAASDEKRIETLISEIKNMFRCMGYGETNPSAYDTAWVARIPAVDGSDNPHFPETVEWILQNQLKDGSWGEGFYFLAYDRILATLACIITLTLWRTGETQVQKGIEFFRTQAGKMEDEADSHRPSGFEIVFPAMLKEAKILGLDLPYDLPFLKQIIEKREAKLKRIPTDVLYALPTTLLYSLEGLQEIVDWQKIMKLQSKDGSFLSSPASTAAVFMRTGNKKCLDFLNFVLKKFGNHVPCHYPLDLFERLWAVDTVERLGIDRHFKEEIKEALDYVYSHWDERGIGWARENPVPDIDDTAMGLRILRLHGYNVSSDVLKTFRDENGEFFCFLGQTQRGVTDMLNVNRCSHVSFPGETIMEEAKLCTERYLRNALENVDAFDKWAFKKNIRGEVEYALKYPWHKSMPRLEARSYIENYGPDDVWLGKTVYMMPYISNEKYLELAKLDFNKVQSIHQTELQDLRRWWKSSGFTDLNFTRERVTEIYFSPASFIFEPEFSKCREVYTKTSNFTVILDDLYDAHGSLDDLKLFTESVKRWDLSLVDQMPQQMKICFVGFYNTFNDIAKEGRERQGRDVLGYIQNVWKVQLEAYTKEAEWSEAKYVPSFNEYIENASVSIALGTVVLISALFTGEVLTDEVLSKIDRESRFLQLMGLTGRLVNDTKTYQAERGQGEVASAIQCYMKDHPKISEEEALQHVYSVMENALEELNREFVNNKIPDIYKRLVFETARIMQLFYMQGDGLTLSHDMEIKEHVKNCLFQPVA

SEQ ID NO: 59 - protein, 14 aa, synthetic construct
AAKARVLXAEAMAA

SEQ ID NO: 60 - protein, 18 aa, synthetic construct
AAAAKARVLXAEAMAAAA

SEQ ID NO: 61 - protein, 13 aa, synthetic construct
AAAALRGGXAAAA

SEQ ID NO: 62 - DNA, 336 bp, synthetic construct
  source 1..336: mol_type = other DNA
atggcggatcgccagggcaccgtgagctttaactttccgcagattaccctgtggcagcgcccgctggtgaccattaaaattggcggccagctgaaagaagcgctgctggataccggcgcggatgataccgtgctggaagaaatgagcctgccgggccgctggaaaccgaaaatgattggcggcattggcggctttattaaagtgcgccagtatgatcagattctgattgaaatttgcggccataaagcgattggcaccgtgctggtgggcccgaccccggtgaacattattggccgcaacctgctgacccagattggctgcaccctgaacttttaa

SEQ ID NO: 63 - protein, 111 aa, synthetic construct
MADRQGTVSFNFPQITLWQRPLVTIKIGGQLKEALLDTGADDTVLEEMSLPGRWKPKMIGGIGGFIKVRQYDQILIEICGHKAIGTVLVGPTPVNIIGRNLLTQIGCTLNF

SEQ ID NO: 64 - DNA, 1158 bp, synthetic construct
  source 1..1158: mol_type = other DNA
atgagtaaaaagcatacagggtacgtgggcttaaaaaaccagggcgctacatgttatatgaattcgctgttacagactttatttttcactaatcagttacgtaaagcggtgtacatgatgcctacggagggggatgactcgtctaaaagcgtcccgcttgccttgcaacgtgtcttttacgagttgcagcactcggacaaacctgtagggactaaaaagttaactaaaagttttggctgggaaactcttgactctttcatgcagcatgacgttcaagaactttgccgtgtgctgttggacaatgtagagaacaaaatgaaaggaacatgcgtagaaggaaccatcccgaagttgttccgcggtaaaatggtgtcatatattcaatgtaaggaagttgactaccgctcggaccgtcgcgaagattactatgatatccagctgagcattaaagggaagaaaaacattttcgagtctttcgttgattacgtcgcggtggagcaactggacggagataacaagtatgacgcaggggagcatggtcttcaagaggccgagaagggcgttaaatttttaacacttccccccgtcctgcatctgcagttaatgcgcttcatgtacgatccccagacggatcaaaacattaaaatcaacgatcgctttgaatttccagaacagttacctttggacgaatttttgcaaaaaacagacccaaaggatccggcaaactacattttacatgcagttttagttcactctggcgacaatcacggagggcactacgttgtttatttaaaccctaaaggtgacggtaagtggtgtaagttcgacgacgacgtagtctctcgttgcacgaaggaggaggcgattgagcataattatggagggcatgatgacgacctttcagttcgtcattgtaccaatgcgtacatgttagtgtatatccgtgaaagcaagttgtcagaggtactgcaagctgtgacagatcatgacatcccgcaacaactggttgaacgccttcaggaggaaaaacgcatcgaagcacagaagcgcaaagaacgtcaagaaggtggaggaggtggtagtggaggaggaggtgggaaagcgccgaagcgcagccgttatacgtacctggaaaaggctatcaaaattcacaactaa

SEQ ID NO: 65 - protein, 385 aa, synthetic construct
MSKKHTGYVGLKNQGATCYMNSLLQTLFFTNQLRKAVYMMPTEGDDSSKSVPLALQRVFYELQHSDKPVGTKKLTKSFGWETLDSFMQHDVQELCRVLLDNVENKMKGTCVEGTIPKLFRGKMVSYIQCKEVDYRSDRREDYYDIQLSIKGKKNIFESFVDYVAVEQLDGDNKYDAGEHGLQEAEKGVKFLTLPPVLHLQLMRFMYDPQTDQNIKINDRFEFPEQLPLDEFLQKTDPKDPANYILHAVLVHSGDNHGGHYVVYLNPKGDGKWCKFDDDVVSRCTKEEAIEHNYGGHDDDLSVRHCTNAYMLVYIRESKLSEVLQAVTDHDIPQQLVERLQEEKRIEAQKRKERQEGGGGGSGGGGGKAPKRSRYTYLEKAIKIHN

SEQ ID NO: 66 - DNA, 951 bp, synthetic construct
  source 1..951: mol_type = other DNA
atggaggttcgtactattaaggtttttaccacagtagacaacattaatctgcatacgcaggtagtagatatgagtatgacgtacggacaacaattcgggcctacctacttagacggagccgacgtaacgaagattaagccacacaatagtcatgagggaaagacattttatgtccttcctaatgacgacactctgcgtgtagaggctttcgaatattaccacacgaccgacccaagtttcttgggacgctatatgtcggcccttaaccataccaagaaatggaagtacccgcaagtcaacgggctgacaagcattaaatgggctgataataattgttatctggctacagcattattaacattgcaacagatcgaacttaaattcaatccacccgctcttcaagacgcctactaccgtgcccgcgccggtgaagcagccaatttctgcgctttaatcttagcttattgtaataaaactgttggggaacttggggacgtacgtgagacgatgtcgtacttgtttcagcatgcaaatctggactcgtgcaaacgtgttctgaacgtggtgtgtaagacgtgcggacagcaacaaactactttgaagggcgtcgaggctgtcatgtatatgggcacgcttagctacgaacaatttaagaagggagttcaaattccttgtacttgcgggaagcaggcaacaaaatatctggttcaacaggaaagtccgttcgttatgatgtctgccccaccagcacaatacgagcttaaacatggaacctttacctgcgcgagtgaatacacgggaaattatcaatgtggccactacaagcacattacgtccaaggaaactttatactgtatcgatggtgccctgttgactaagtcgtcggagtataaaggtccgattacagatgtattctacaaagagaactcttacaccacgacgatcaagtaa

SEQ ID NO: 67 - protein, 316 aa, synthetic construct
MEVRTIKVFTTVDNINLHTQVVDMSMTYGQQFGPTYLDGADVTKIKPHNSHEGKTFYVLPNDDTLRVEAFEYYHTTDPSFLGRYMSALNHTKKWKYPQVNGLTSIKWADNNCYLATALLTLQQIELKFNPPALQDAYYRARAGEAANFCALILAYCNKTVGELGDVRETMSYLFQHANLDSCKRVLNVVCKTCGQQQTTLKGVEAVMYMGTLSYEQFKKGVQIPCTCGKQATKYLVQQESPFVMMSAPPAQYELKHGTFTCASEYTGNYQCGHYKHITSKETLYCIDGALLTKSSEYKGPITDVFYKENSYTTTIK

SEQ ID NO: 68 - DNA, 924 bp, synthetic construct
  source 1..924: mol_type = other DNA
atgtcggggttccgtaaaatggctttccccagtggcaaggtagagggatgtatggtccaagtgacctgtggaacgaccacgttaaatgggttgtggcttgatgatgtagtttattgtcctcgccacgttatttgcacaagtgaggatatgttgaatcctaattatgaggatctgttaatccgtaaatcgaatcataattttcttgtccaagcgggaaatgttcaattgcgtgttatcggacactctatgcagaactgcgtcctgaagttgaaagttgatacggccaatccgaagacgcctaagtacaagtttgtgcgcattcaacctggacagacattttctgtactggcgtgctacaacggcagccccagcggtgtatatcagtgtgcaatgcgcccgaactttacaatcaaagggtcgtttttgaatggtagttgcggctcagttggtttcaacattgattatgattgtgtctccttttgttacatgcaccatatggagctgccaaccggcgtgcatgccggcacggatttggagggcaatttttacggaccctttgtggaccgccaaacagcccaggccgcaggtactgataccactatcaccgtcaacgtgcttgcttggctgtacgcggcggtgatcaatggagaccgctggttccttaatcgttttaccacaacacttaatgacttcaacttggtagcaatgaaatacaactacgagcctcttacgcaggaccatgttgacatcttgggtccgctgtctgcacagactgggattgctgtacttgatatgtgtgcaagcttaaaggaacttcttcaaaacggtatgaatggacgtactatccttgggtcggccttattagaagacgagttcacaccgtttgacgttgtccgccaatgtagcggcgtaactttccaataa

SEQ ID NO: 69 - protein, 307 aa, synthetic construct
MSGFRKMAFPSGKVEGCMVQVTCGTTTLNGLWLDDVVYCPRHVICTSEDMLNPNYEDLLIRKSNHNFLVQAGNVQLRVIGHSMQNCVLKLKVDTANPKTPKYKFVRIQPGQTFSVLACYNGSPSGVYQCAMRPNFTIKGSFLNGSCGSVGFNIDYDCVSFCYMHHMELPTGVHAGTDLEGNFYGPFVDRQTAQAAGTDTTITVNVLAWLYAAVINGDRWFLNRFTTTLNDFNLVAMKYNYEPLTQDHVDILGPLSAQTGIAVLDMCASLKELLQNGMNGRTILGSALLEDEFTPFDVVRQCSGVTFQ

SEQ ID NO: 70 - protein, 378 aa, synthetic construct
MAARAPVHLRGFIARVALNKKNLHARHKLDTDIDKYYYTLHNVIIPDFMDMVKEIPGYPERIKKCVAHTTPSYFEGWAFSTELIYKTVADKQHQTERNLEKCRIIRALMDMSYAMAGILDDYVDKGEFRRGKKVWASVCEGGQEAAIYDSIAVTYLMSLMVKRHFGTDPGYSKLIELFNMVPGTAAIGNTLDILDRHDTNYYDDTMWKHSVQNKAANTVFPAATAGLIHAGVLCDDLLDRTSEVFGYTGHLFQVWDDFMEHYAVKEQSGKGAPDTKYNAKTWATLTAMAHFNEAQAKEFKACYGSTDPAKRSRVRELYDEVNLRGLYIDYLRNTYMVVEEKISKIPDPRIQSACRSYMDWLLVEPPQDEEEAESVLNN

SEQ ID NO: 71 - protein, 333 aa, synthetic construct
MIDVPVLRVPFPARINPHSTTLGARLRHWLQRSGLATPELNRQFERARFDLLVASLYPAAGAEELRTLAELVAWMFVYDDHFDVHRLGGSPANAARAADQVSAVLAGAPASGPLLTALSDLRRNRLSAVPAPLRQRLLGHLDDYCRSLVRELEFRAANRIPAPGAYFDLRMNTFAWPVLADLAEFADGIVLPAAVRDSPEFAALLSTSGHLMIVIQDLRSLDRELANGESHNVVLSLREERRCSLPEAVELAHRIFVERLGEFLSRRDAVLVAFDRLALDDRARRAAASYVVGLEHLLSGHLAWYARTDRYGRAGALESLGGTDDLIFEGMDR

SEQ ID NO: 72 - protein, 817 aa, synthetic construct
MAGVSAVSKVSSLVCDLSSTSGLIRRTANPHPNVWGYDLVHSLKSPYIDSSYRERAEVLVSEIKAMLNPAITGDGESMITPSAYDTAWVARVPAIDGSARPQFPQTVDWILKNQLKDGSWGIQSHFLLSDRLLATLSCVLVLLKWNVGDLQVEQGIEFIKSNLELVKDETDQDSLVTDFEIIFPSLLREAQSLRLGLPYDLPYIHLLQTKRQERLAKLSREEIYAVPSPLLYSLEGIQDIVEWERIMEVQSQDGSFLSSPASTACVFMHTGDAKCLEFLNSVMIKFGNFVPCLYPVDLLERLLIVDNIVRLGIYRHFEKEIKEALDYVYRHWNERGIGWGRLNPIADLETTALGFRLLRLHRYNVSPAIFDNFKDANGKFICSTGQFNKDVASMLNLYRASQLAFPGENILDEAKSFATKYLREALEKSETSSAWNNKQNLSQEIKYALKTSWHASVPRVEAKRYCQVYRPDYARIAKCVYKLPYVNNEKFLELGKLDFNIIQSIHQEEMKNVTSWFRDSGLPLFTFARERPLEFYFLVAAGTYEPQYAKCRFLFTKVACLQTVLDDMYDTYGTLDELKLFTEAVRRWDLSFTENLPDYMKLCYQIYYDIVHEVAWEAEKEQGRELVSFFRKGWEDYLLGYYEEAEWLAAEYVPTLDEYIKNGITSIGQRILLLSGVLIMDGQLLSQEALEKVDYPGRRVLTELNSLISRLADDTKTYKAEKARGELASSIECYMKDHPECTEEEALDHIYSILEPAVKELTREFLKPDDVPFACKKMLFEETRVTMVIFKDGDGFGVSKLEVKDHIKECLIEPLPL

SEQ ID NO: 73 - DNA, 867 bp, synthetic construct
  source 1..867: mol_type = other DNA
atgggctccaagccgcagactcagggcctggccaaggatgcctgggagatccctcgggagtcgctgcggctggaggtcaagctgggccagggctgctttggcgaggtgtggatggggacctggaacggtaccaccagggtggccatcaaaaccctgaagcctggcacgatgtctccagaggccttcctgcaggaggcccaggtcatgaagaagctgaggcatgagaagctggtgcagttgtatgctgtggtttcagaggagcccatttacatcgtcacggagtacatgagcaaggggagtttgctggactttctcaagggggagacaggcaagtacctgcggctgcctcagctggtggacatggctgctcagatcgcctcaggcatggcgtacgtggagcggatgaactacgtccaccgggaccttcgtgcagccaacatcctggtgggagagaacctggtgtgcaaagtggccgactttgggctggctcggctcattgaagacaatgagtacacggcgcggcaaggtgccaaattccccatcaagtggacggctccagaagctgccctctatggccgcttcaccatcaagtcggacgtgtggtccttcgggatcctgctgactgagctcaccacaaagggacgggtgccctaccctgggatggtgaaccgcgaggtgctggaccaggtggagcggggctaccggatgccctgcccgccggagtgtcccgagtccctgcacgacctcatgtgccagtgctggcggaaggagcctgaggagcggcccaccttcgagtacctgcaggccttcctggaggactacttcacgtccaccgagccccagtaccagcccggggagaacctctaa

SEQ ID NO: 74 - protein, 288 aa, synthetic construct
MGSKPQTQGLAKDAWEIPRESLRLEVKLGQGCFGEVWMGTWNGTTRVAIKTLKPGTMSPEAFLQEAQVMKKLRHEKLVQLYAVVSEEPIYIVTEYMSKGSLLDFLKGETGKYLRLPQLVDMAAQIASGMAYVERMNYVHRDLRAANILVGENLVCKVADFGLARLIEDNEYTARQGAKFPIKWTAPEAALYGRFTIKSDVWSFGILLTELTTKGRVPYPGMVNREVLDQVERGYRMPCPPECPESLHDLMCQCWRKEPEERPTFEYLQAFLEDYFTSTEPQYQPGENL

SEQ ID NO: 75 - DNA, 1137 bp, synthetic construct
  source 1..1137: mol_type = other DNA
atggtggactacagcgtgtgggaccacattgaggtgtctgatgatgaagacgagacgcaccccaacatcgacacggccagtctcttccgctggcggcatcaggcccgggtggaacgcatggagcagttccagaaggagaaggaggaactggacaggggctgccgcgagtgcaagcgcaaggtggccgagtgccagaggaaactgaaggagctggaggtggccgagggcggcaaggcagagctggagcgcctgcaggccgaggcacagcagctgcgcaaggaggagcggagctgggagcagaagctggaggagatgcgcaagaaggagaagagcatgccctggaacgtggacacgctcagcaaagacggcttcagcaagagcatggtaaataccaagcccgagaagacggaggaggactcagaggaggtgagggagcagaaacacaagaccttcgtggaaaaatacgagaaacagatcaagcactttggcatgcttcgccgctgggatgacagccaaaagtacctgtcagacaacgtccacctggtgtgcgaggagacagccaattacctggtcatttggtgcattgacctagaggtggaggagaaatgtgcactcatggagcaggtggcccaccagacaatcgtcatgcaatttatcctggagctggccaagagcctaaaggtggacccccgggcctgcttccggcagttcttcactaagattaagacagccgatcgccagtacatggagggcttcaacgacgagctggaagccttcaaggagcgtgtgcggggccgtgccaagctgcgcatcgagaaggccatgaaggagtacgaggaggaggagcgcaagaagcggctcggccccggcggcctggaccccgtcgaggtctacgagtccctccctgaggaactccagaagtgcttcgatgtgaaggacgtgcagatgctgcaggacgccatcagcaagatggaccccaccgacgcaaagtaccacatgcagcgctgcattgactctggcctctgggtccccaactctaaggccagcgaggccaaggagggagaggaggcaggtcctggggacccattactggaagctgttcccaagacgggcgatgagaaggatgtcagtgtgtaa

SEQ ID NO: 76 - protein, 378 aa, synthetic construct
MVDYSVWDHIEVSDDEDETHPNIDTASLFRWRHQARVERMEQFQKEKEELDRGCRECKRKVAECQRKLKELEVAEGGKAELERLQAEAQQLRKEERSWEQKLEEMRKKEKSMPWNVDTLSKDGFSKSMVNTKPEKTEEDSEEVREQKHKTFVEKYEKQIKHFGMLRRWDDSQKYLSDNVHLVCEETANYLVIWCIDLEVEEKCALMEQVAHQTIVMQFILELAKSLKVDPRACFRQFFTKIKTADRQYMEGFNDELEAFKERVRGRAKLRIEKAMKEYEEEERKKRLGPGGLDPVEVYESLPEELQKCFDVKDVQMLQDAISKMDPTDAKYHMQRCIDSGLWVPNSKASEAKEGEEAGPGDPLLEAVPKTGDEKDVSV

SEQ ID NO: 77 - DNA, 729 bp, synthetic construct
  source 1..729: mol_type = other DNA
atgggtacagacatgtggatcgagcgtaccgcggacatttcatgggaaagtgacgcggagattactggctcgtcagaacgtgttgatgttcgccttgatgacgatggcaactttcagttgatgaacgaccccggtgcagggggaggaggcagtggcggtgggggtggggtgttgtgggatactccgtcacctaaagaatacaaaaaaggcgataccactactggcgtgtaccgcatcatgacccgcgggctgcttgggtcatatcaagccggggcaggcgtaatggtcgagggagtatttcatactttgtggcacacgactaagggggcggctcttatgtctggcgaaggtcgcttagatccgtattggggaagcgtcaaagaggatcgcttatgctacgggggaccttggaaattacaacataagtggaacggtcaggacgaagtccaaatgatcgtggttgaacccggcaagaacgtgaaaaatgttcgcacaaagcctggggtgttcaagacgcccgagggcgaaatcggtgcggtgacattggatttccctactggcacctctggatcaccaatcgttgataaaaacggcgatgtaatcgggttatacggaaatggggttattatgcctaatggatcatatatcagtgccattgttcagggcaaacgtatggatgagcctatccccgccggtttcgaaccggaaatgttaggctcacgttcttaa

SEQ ID NO: 78 - protein, 242 aa, synthetic construct
MGTDMWIERTADISWESDAEITGSSERVDVRLDDDGNFQLMNDPGAGGGGSGGGGGVLWDTPSPKEYKKGDTTTGVYRIMTRGLLGSYQAGAGVMVEGVFHTLWHTTKGAALMSGEGRLDPYWGSVKEDRLCYGGPWKLQHKWNGQDEVQMIVVEPGKNVKNVRTKPGVFKTPEGEIGAVTLDFPTGTSGSPIVDKNGDVIGLYGNGVIMPNGSYISAIVQGKRMDEPIPAGFEPEMLGSRS

SEQ ID NO: 79 - DNA, 792 bp, synthetic construct
  source 1..792: mol_type = other DNA
atgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaatga

SEQ ID NO: 80 - protein, 263 aa, synthetic construct
MREAVIAEVSTQLSEVVGVIERHLEPTLLAVHLYGSAVDGGLKPHSDIDLLVTVTVRLDETTRRALINDLLETSASPGESEILRAVEVTIVVHDDIIPWRYPAKRELQFGEWQRNDILAGIFEPATIDIDLAILLTKAREHSVALVGPAAEELFDPVPEQDLFEALNETLTLWNSPPDWAGDERNVVLTLSRIWYSAVTGKIAPKDVAADWAMERLPAQYQPVILEARQAYLGQEEDRLASRADQLEEFVHYVKGEITKVVGK

SEQ ID NO: 81 - protein, 7 aa, synthetic construct
TSAVLQX

SEQ ID NO: 82 - DNA, 238 bp, synthetic construct
  source 1..238: mol_type = other DNA
agaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcg

SEQ ID NO: 83 - protein, 160 aa, synthetic construct
TTCTAGAGCACAGCTAACACCACGTCGTCCCTATCTGCTGCCCTAGGTCTATGAGTGGTTGCTGGATAACTTTACGGGCATGCATAAGGCTCGGTATCTATATTCAGGGAGACCACAACGGTTTCCCTCTACAAATAATTTTGTTTAACTTTTACTAGAG

SEQ ID NO: 84 - DNA, 57 bp, synthetic construct
  source 1..57: mol_type = other DNA
cattaggcaccccgggctttactcgtaaagcttccggcgcgtatgttgtgtcgaccg

SEQ ID NO: 85 - DNA, 160 bp, synthetic construct
  source 1..160: mol_type = other DNA
ttctagagcacagctaacaccacgtcgtccctatctgctgccctaggtctatgagtggttgctggataactttacgggcatgcataaggctcggtatctatattcagggagaccacaacggtttccctctacaaataattttgtttaacttttactagag

SEQ ID NO: 86 - DNA, 633 bp, synthetic construct
  source 1..633: mol_type = other DNA
atgagtatcagcagcagggtaaaaagcaaaagaattcagcttggacttaaccaggctgaacttgctcaaaaggtggggactacccagcagtctatagagcagctcgaaaacggtaaaactaagcgaccacgctttttaccagaacttgcgtcagctcttggcgtaagtgttgactggctgctcaatggcacctctgattcgaatgttagatttgttgggcacgttgagcccaaagggaaatatccattgattagcatggttagagctcgttcgtggtgtgaagcttgtgaaccctacgatatcaaggacattgatgaatggtatgacagtgacgttaacttattaggcaatggattctggctgaaggttgaaggtgattccatgacctcacctgtaggtcaaagcatccctgaaggtcatatggtgttagtagatactggacgggagccagtgaatggaagccttgttgtagccaaactgactgacgcgaacgaagcaacattcaagaaactggtcatagatggcggtcagaagtacctgaaaggcctgaatccttcatggcctatgactcctatcaacggaaactgcaagattatcggtgttgtcgtggaagcgagggtaaaattcgtagac

SEQ ID NO: 87 - protein, 211 aa, synthetic construct
MSISSRVKSKRIQLGLNQAELAQKVGTTQQSIEQLENGKTKRPRFLPELASALGVSVDWLLNGTSDSNVRFVGHVEPKGKYPLISMVRARSWCEACEPYDIKDIDEWYDSDVNLLGNGFWLKVEGDSMTSPVGQSIPEGHMVLVDTGREPVNGSLVVAKLTDANEATFKKLVIDGGQKYLKGLNPSWPMTPINGNCKIIGVVVEARVKFVD

SEQ ID NO: 88 - DNA, 744 bp, synthetic construct
  source 1..744: mol_type = other DNA
atgcagggttctgtgacagagtttctaaaaccgcgcctggttgatatcgagcaagtgagttcgacgcacgccaaggtgacccttgagcctttagagcgtggctttggccatactctgggtaacgcactgcgccgtattctgctctcatcgatgccgggttgcgcggtgaccgaggttgagattgatggtgtactacatgagtacagcaccaaagaaggcgttcaggaagatatcctggaaatcctgctcaacctgaaagggctggcggtgagagttcagggcaaagatgaagttattcttaccttgaataaatctggcattggccctgtgactgcagccgatatcacccacgacggtgatgtcgaaatcgtcaagccgcagcacgtgatctgccacctgaccgatgagaacgcgtctattagcatgcgtatcaaagttcagcgcggtcgtggttatgtgccggcttctacccgaattcattcggaagaagatgagcgcccaatcggccgtctgctggtcgacgcatgctacagccctgtggagcgtattgcctacaatgttgaagcagcgcgtgtagaacagcgtaccgacctggacaagctggtcatcgaaatggaaaccaacggcacaatcgatcctgaagaggcgattcgtcgtgcggcaaccattctggctgaacaactggaagctttcgttgacttacgtgatgtacgtcagcctgaagtgaaagaagagaaaccagag

SEQ ID NO: 89 - protein, 248 aa, synthetic construct
MQGSVTEFLKPRLVDIEQVSSTHAKVTLEPLERGFGHTLGNALRRILLSSMPGCAVTEVEIDGVLHEYSTKEGVQEDILEILLNLKGLAVRVQGKDEVILTLNKSGIGPVTAADITHDGDVEIVKPQHVICHLTDENASISMRIKVQRGRGYVPASTRIHSEEDERPIGRLLVDACYSPVERIAYNVEAARVEQRTDLDKLVIEMETNGTIDPEEAIRRAATILAEQLEAFVDLRDVRQPEVKEEKPE

SEQ ID NO: 90 - DNA, 294 bp, synthetic construct
  source 1..294: mol_type = other DNA
tggtattttgggaagatcactcgtcgggagtccgagcggctgctgctcaaccccgaaaacccccggggaaccttcttggtccgggagagcgagacggtaaaaggtgcctatgccctctccgtttctgactttgacaacgccaaggggctcaatgtgaaacactacctgatccgcaagctggacagcggcggcttctacatcacctcacgcacacagttcagcagcctgcagcagctggtggcctactactccaaacatgctgatggcttgtgccaccgcctgaccaacgtctgc

SEQ ID NO: 91 - protein, 98 aa, synthetic construct
WYFGKITRRESERLLLNPENPRGTFLVRESETVKGAYALSVSDFDNAKGLNVKHYLIRKLDSGGFYITSRTQFSSLQQLVAYYSKHADGLCHRLTNVC

SEQ ID NO: 92 - DNA, 300 bp, synthetic construct
  source 1..300: mol_type = other DNA
agtctggaaaaacacagctggtatcatggccctgtgagccgtaacgcggccgaatacctgctgagctctggcattaatggttcttttctggttcgtgaaagtgaaagtagcccgggccagcgcagcatttctctgcgttatgaaggtcgcgtgtatcactaccgtatcaacaccgccagcgatggcaaactgtacgtttctagtgaatctcgcttcaataccctggcagaactggtgcatcaccatagcacggttgcggatggtctgatcaccacgctgcattatccggcgccgaaacgc

SEQ ID NO: 93 - protein, 100 aa, synthetic construct
SLEKHSWYHGPVSRNAAEYLLSSGINGSFLVRESESSPGQRSISLRYEGRVYHYRINTASDGKLYVSSESRFNTLAELVHHHSTVADGLITTLHYPAPKR

SEQ ID NO: 94 - DNA, 333 bp, synthetic construct
  source 1..333: mol_type = other DNA
gaacaaaagcttatttctgaagaggacttgggcagctctgtgagtagcgttccgaccaaactggaagtggttgcagcaaccccgacgagcctgctgatttcttgggatgccccgatgtctagtagctctgtgtattactatcgtatcacctacggtgaaacgggcggtaacagcccggtgcaggaatttacggttccgtatagtagctctaccgcgacgattagtggcctgagcccgggtgtggattacaccatcacggtttatgcatggggcgaagatagcgcgggttacatgttcatgtattctccgattagtatcaattaccgcacctgc

SEQ ID NO: 95 - protein, 111 aa, synthetic construct
EQKLISEEDLGSSVSSVPTKLEVVAATPTSLLISWDAPMSSSSVYYYRITYGETGGNSPVQEFTVPYSSSTATISGLSPGVDYTITVYAWGEDSAGYMFMYSPISINYRTC

SEQ ID NO: 96 - DNA, 33 bp, synthetic construct
  source 1..33: mol_type = other DNA
gaaccgcagtatgaagaaattccgatttatctg

SEQ ID NO: 97 - protein, 11 aa, synthetic construct
EPQYEEIPIYL

SEQ ID NO: 98 - DNA, 33 bp, synthetic construct
  source 1..33: mol_type = other DNA
gaaccgcagtttgaagaaattccgatttatctg

SEQ ID NO: 99 - protein, 11 aa, synthetic construct
EPQFEEIPIYL

SEQ ID NO: 100 - DNA, 25 bp, synthetic construct
  source 1..25: mol_type = other DNA
ctcagaactttgcaaggaggtattg

SEQ ID NO: 101 - DNA, 29 bp, synthetic construct
  source 1..29: mol_type = other DNA
tatccacagtaacataggggaggattaat

SEQ ID NO: 102 - DNA, 38 bp, synthetic construct
  source 1..38: mol_type = other DNA
aggtaatttatttaagatacacataaggaggatattaa

SEQ ID NO: 103 - DNA, 27 bp, synthetic construct
  source 1..27: mol_type = other DNA
tcgacagcagccaataaggaggtatta

SEQ ID NO: 104 - DNA, 27 bp, synthetic construct
  source 1..27: mol_type = other DNA
tcgacagcagcggataaggaggtatta

SEQ ID NO: 105 - DNA, 27 bp, synthetic construct
  source 1..27: mol_type = other DNA
acaattcatatcctaagcgcttcttaa

SEQ ID NO: 106 - DNA, 27 bp, synthetic construct
  source 1..27: mol_type = other DNA
atttgccaccattaaaggaggttccaa

SEQ ID NO: 107 - DNA, 22 bp, synthetic construct
  source 1..22: mol_type = other DNA
gtgcagtaaggaggaaaaaaaa

SEQ ID NO: 108 - DNA, 22 bp, synthetic construct
  source 1..22: mol_type = other DNA
gggccgacttgcggtataataa

SEQ ID NO: 109 - DNA, 24 bp, synthetic construct
  source 1..24: mol_type = other DNA
gcagttttacaatcagggttccgt

SEQ ID NO: 110 - DNA, 27 bp, synthetic construct
  source 1..27: mol_type = other DNA
aaagctcgcgtactggccgaagccatg

SEQ ID NO: 111 - DNA, 12 bp, synthetic construct
  source 1..12: mol_type = other DNA
ttacgtgggggg

SEQ ID NO: 112 - DNA, 2124 bp, synthetic construct
  source 1..2124: mol_type = other DNA
atgaaatttggaaactttttgcttacataccaacctccccaattttcccaaacagaggtaatgaaacgtttggttaaattaggtcgcatctctgaggagtgtggttttgataccgtatggttactggagcatcatttcacggagtttggtttgcttggtaacccttatgtcgctgctgcatatttacttggcgcgactaaaaaattgaatgtaggaactgccgctattgttcttcccacagcccatccagtacgccaacttgaagatgtgaatttattggatcaaatgtcaaaaggacgatttcggtttggtatttgccgagggctttacaacaaggactttcgcgtattcggcacagatatgaataacagtcgcgccttagcggaatgctggtacgggctgataaagaatggcatgacagagggatatatggaagctgataatgaacatatcaagttccataaggtaaaagtaaaccccgcggcgtatagcagaggtggcgcaccggtttatgtggtggctgaatcagcttcgacgactgagtgggctgctcaatttggcctaccgatgatattaagttggattataaatactaacgaaaagaaagcacaacttgagctttataatgaagtggctcaagaatatgggcacgatattcataatatcgaccattgcttatcatatataacatctgtagatcatgactcaattaaagcgaaagagatttgccggaaatttctggggcattggtatgattcttatgtgaatgctacgactatttttgatgattcagaccaaacaagaggttatgatttcaataaagggcagtggcgtgactttgtattaaaaggacataaagatactaatcgccgtattgattacagttacgaaatcaatcccgtgggaacgccgcaggaatgtattgacataattcaaaaagacattgatgctacaggaatatcaaatatttgttgtggatttgaagctaatggaacagtagacgaaattattgcttccatgaagctcttccagtctgatgtcatgccatttcttaaagaaaaacaacgttcgctattatattatggcggtggcggtagcggcggtggcggtagcggcggtggcggtagcggcggtggcggtagcaaatttggattgttcttccttaacttcatcaattcaacaactgttcaagaacagagtatagttcgcatgcaggaaataacggagtatgttgataagttgaattttgaacagattttagtgtatgaaaatcatttttcagataatggtgttgtcggcgctcctctgactgtttctggttttctgctcggtttaacagagaaaattaaaattggttcattaaatcacatcattacaactcatcatcctgtccgcatagcggaggaagcttgcttattggatcagttaagtgaagggagatttattttagggtttagtgattgcgaaaaaaaagatgaaatgcatttttttaatcgcccggttgaatatcaacagcaactatttgaagagtgttatgaaatcattaacgatgctttaacaacaggctattgtaatccagataacgatttttatagcttccctaaaatatctgtaaatccccatgcttatacgccaggcggacctcggaaatatgtaacagcaaccagtcatcatattgttgagtgggcggccaaaaaaggtattcctctcatctttaagtgggatgattctaatgatgttagatatgaatatgctgaaagatataaagccgttgcggataaatatgacgttgacctatcagagatagaccatcagttaatgatattagttaactataacgaagatagtaataaagctaaacaagagacgcgtgcatttattagtgattatgttcttgaaatgcaccctaatgaaaatttcgaaaataaacttgaagaaataattgcagaaaacgctgtcggaaattatacggagtgtataactgcggctaagttggcaattgaaaagtgtggtgcgaaaagtgtattgctgtcctttgaaccaatgaatgatttgatgagccaaaaaaatgtaatcaatattgttgatgataatattaagaagtaccacacggaatatacctaa

SEQ ID NO: 113 - DNA, 14 bp, synthetic construct
  source 1..14: mol_type = other DNA
acaagaaagtttgt

SEQ ID NO: 114 - DNA, 14 bp, synthetic construct
  source 1..14: mol_type = other DNA
acaagatacattgt

SEQ ID NO: 115 - DNA, 28 bp, synthetic construct
  source 1..28: mol_type = other DNA
aacaaacagacaatctggtctgtttgta

SEQ ID NO: 116 - DNA, 30 bp, synthetic construct
  source 1..30: mol_type = other DNA
atgatacgaaacgtaccgtatcgttaaggt

SEQ ID NO: 117 - DNA, 17 bp, synthetic construct
  source 1..17: mol_type = other DNA
tatcaccgcaagggata

SEQ ID NO: 118 - DNA, 44 bp, synthetic construct
  source 1..44: mol_type = other DNA
cccatgcgtgtcgtataagtccgctaacattgtcatcaagatcg

SEQ ID NO: 119 - DNA, 44 bp, synthetic construct
  source 1..44: mol_type = other DNA
cgatcttgatgacaatgttagcggacttatacgacacgcatggg

SEQ ID NO: 120 - DNA, 48 bp, synthetic construct
  source 1..48: mol_type = other DNA
ccactaaattctggttctgaaatctgcgcggccatccagaagtaaaat

SEQ ID NO: 121 - DNA, 48 bp, synthetic construct
  source 1..48: mol_type = other DNA
attttacttctggatggccgcgcagatttcagaaccagaatttagtgg

SEQ ID NO: 122 - DNA, 37 bp, synthetic construct
  source 1..37: mol_type = other DNA
gttatacataaactgaatgcaccgcgccaggacgtgg

SEQ ID NO: 123 - DNA, 37 bp, synthetic construct
  source 1..37: mol_type = other DNA
ccacgtcctggcgcggtgcattcagtttatgtataac

SEQ ID NO: 124 - DNA, 40 bp, synthetic construct
  source 1..40: mol_type = other DNA
cctgcaaatacgcttccaggcagcgttcccaagcgttttt

SEQ ID NO: 125 - DNA, 40 bp, synthetic construct
  source 1..40: mol_type = other DNA
aaaaacgcttgggaacgctgcctggaagcgtatttgcagg

SEQ ID NO: 126 - DNA, 40 bp, synthetic construct
  source 1..40: mol_type = other DNA
cagcaacgggatcagattatatacacacataccggtgttg

SEQ ID NO: 127 - DNA, 40 bp, synthetic construct
  source 1..40: mol_type = other DNA
caacaccggtatgtgtgtatataatctgatcccgttgctg

SEQ ID NO: 128 - DNA, 43 bp, synthetic construct
  source 1..43: mol_type = other DNA
agcagcaacgggatcagattgcctacacacataccggtgttgg

SEQ ID NO: 129 - DNA, 43 bp, synthetic construct
  source 1..43: mol_type = other DNA
ccaacaccggtatgtgtgtaggcaatctgatcccgttgctgct

SEQ ID NO: 130 - DNA, 45 bp, synthetic construct
  source 1..45: mol_type = other DNA
aagcagcaacgggatcagattttttacacacataccggtgttggg

SEQ ID NO: 131 - DNA, 45 bp, synthetic construct
  source 1..45: mol_type = other DNA
cccaacaccggtatgtgtgtaaaaaatctgatcccgttgctgctt

SEQ ID NO: 132 - DNA, 45 bp, synthetic construct
  source 1..45: mol_type = other DNA
aagcagcaacgggatcagattggttacacacataccggtgttggg

SEQ ID NO: 133 - DNA, 45 bp, synthetic construct
  source 1..45: mol_type = other DNA
cccaacaccggtatgtgtgtaaccaatctgatcccgttgctgctt

SEQ ID NO: 134 - DNA, 42 bp, synthetic construct
  source 1..42: mol_type = other DNA
attaagtacacacataccaatgttgggggtgccattgttcag

SEQ ID NO: 135 - DNA, 42 bp, synthetic construct
  source 1..42: mol_type = other DNA
ctgaacaatggcacccccaacattggtatgtgtgtacttaat

SEQ ID NO: 136 - DNA, 42 bp, synthetic construct
  source 1..42: mol_type = other DNA
cgcatcatcgaccagtcgcagagccagttcaatcagatggtg

SEQ ID NO: 137 - DNA, 42 bp, synthetic construct
  source 1..42: mol_type = other DNA
caccatctgattgaactggctctgcgactggtcgatgatgcg

SEQ ID NO: 138 - DNA, 25 bp, synthetic construct
  source 1..25: mol_type = other DNA
aacaatttcacacaggaaacagacc

SEQ ID NO: 139 - DNA, 20 bp, synthetic construct
  source 1..20: mol_type = other DNA
gcctgcaggtcgactctaga

SEQ ID NO: 140 - DNA, 43 bp, synthetic construct
  source 1..43: mol_type = other DNA
agctgaaagaagcgctgctgaacaccggcgcggatgataccgt

SEQ ID NO: 141 - DNA, 43 bp, synthetic construct
  source 1..43: mol_type = other DNA
acggtatcatccgcgccggtgttcagcagcgcttctttcagct

SEQ ID NO: 142 - DNA, 47 bp, synthetic construct
  source 1..47: mol_type = other DNA
tatcctcacttgtgcaaataactgcgcgaggacaataaactacatca

SEQ ID NO: 143 - DNA, 47 bp, synthetic construct
  source 1..47: mol_type = other DNA
tgatgtagtttattgtcctcgcgcagttatttgcacaagtgaggata

SEQ ID NO: 144 - DNA, 45 bp, synthetic construct
  source 1..45: mol_type = other DNA
acgggcccgttgtggtgcacagcagtgcaggcatcggcaggtctg

SEQ ID NO: 145 - DNA, 45 bp, synthetic construct
  source 1..45: mol_type = other DNA
cagacctgccgatgcctgcactgctgtgcaccacaacgggcccgt

SEQ ID NO: 146 - DNA, 39 bp, synthetic construct
  source 1..39: mol_type = other DNA
cagagagaaggtgccagacatcccaatgcctgcactaca

SEQ ID NO: 147 - DNA, 39 bp, synthetic construct
  source 1..39: mol_type = other DNA
tgtagtgcaggcattgggatgtctggcaccttctctctg

SEQ ID NO: 148 - DNA, 60 bp, synthetic construct
  source 1..60: mol_type = other DNA
gctgtgatcaaatggcagtatgtccttcgctctgttctttttaacattttcttctttttc

SEQ ID NO: 149 - DNA, 60 bp, synthetic construct
  source 1..60: mol_type = other DNA
gaaaaagaagaaaatgttaaaaagaacagagcgaaggacatactgccatttgatcacagc

SEQ ID NO: 150 - DNA, 747 bp, synthetic construct
  source 1..747: mol_type = other DNA
atggggtcgcatatgcttgaggcggacttggagcttgaacgcgccgctgatgtacgttgggaggagcaagcagagatcagcgggtcctctcctattctgagcattacgatcagcgaggatggttcaatgagtatcaagaatgaagaggaggagcaaactttgggaggcggcggatcgggtgggggaggcgctggtgtattgtgggatgtgccgtcaccccctccggttggaaaagcggaacttgaggatggcgcttatcgcatcaagcaaaaaggcatcttaggatacagccaaattggggctggagtatacaaggagggcacgttccatacgatgtggcatgttacccgtggcgcggtattaatgcacaagggaaaacgtatcgagccttcgtgggcagatgtgaaaaaggatttgatttcatatggcggtggttggaaattggagggtgaatggaaggaaggtgaggaggttcaggttcttgccttagaacccggaaaaaacccccgcgccgttcagacaaaacctgggttatttaaaactaacactggtacgattggggccgtatcgttagatttcagccctgggactagcggttctccgatcgtcgacaaaaagggaaaagtagtgggcttatatgggaatggggtagtgactcgttctggggcatacgtaagcgcaattgcaaacactgagaaatcgattgaggataatcctgagatcgaggatgacattttccgtaagtaa

SEQ ID NO: 151 - protein, 248 aa, synthetic construct
MGSHMLEADLELERAADVRWEEQAEISGSSPILSITISEDGSMSIKNEEEEQTLGGGGSGGGGAGVLWDVPSPPPVGKAELEDGAYRIKQKGILGYSQIGAGVYKEGTFHTMWHVTRGAVLMHKGKRIEPSWADVKKDLISYGGGWKLEGEWKEGEEVQVLALEPGKNPRAVQTKPGLFKTNTGTIGAVSLDFSPGTSGSPIVDKKGKVVGLYGNGVVTRSGAYVSAIANTEKSIEDNPEIEDDIFRK

SEQ ID NO: 152 - DNA, 609 bp, synthetic construct
  source 1..609: mol_type = other DNA
atgagcccgaaacgtcgtacccaggcagaacgtgcaatggaaacccagggtaaactgattgcagcagcactgggtgttctgcgtgaaaaaggttatgcaggttttcgtattgcagatgttccgggtgcagccggtgttagccgtggtgcacagagccatcattttccgaccaaactggaactgctgctggcaacctttgaatggctgtatgagcagattaccgaacgtagccgtgcacgtctggcaaaactgaaaccggaagatgatgttattcagcagatgctggatgatgcagcagaattttttctggatgatgattttagcatcggcctggatctgattgttgcagcagatcgtgatccggcactgcgtgaaggtattcagcgtaccgttgaacgtaatcgttttgttgttgaagatatgtggctgggtgtgctggtgagccgtggtctgagccgtgatgatgccgaagatattctgtggctgatttttaacagcgttcgtggtctggtagttcgtagcctgtggcagaaagataaagaacgttttgaacgtgtgcgtaatagcaccctggaaattgcacgtgaacgttatgcaaaattcaaacgt

SEQ ID NO: 153 - protein, 203 aa, synthetic construct
MSPKRRTQAERAMETQGKLIAAALGVLREKGYAGFRIADVPGAAGVSRGAQSHHFPTKLELLLATFEWLYEQITERSRARLAKLKPEDDVIQQMLDDAAEFFLDDDFSIGLDLIVAADRDPALREGIQRTVERNRFVVEDMWLGVLVSRGLSRDDAEDILWLIFNSVRGLVVRSLWQKDKERFERVRNSTLEIARERYAKFKR

SEQ ID NO: 154 - DNA, 600 bp, synthetic construct
  source 1..600: mol_type = other DNA
atggcacgtaccccgagccgtagcagcattggtagcctgcgtagtccgcatacccataaagcaattctgaccagcaccattgaaatcctgaaagaatgtggttatagcggtctgagcattgaaagcgtggcacgtcgcgccggtgcaggcaaaccgaccatttatcgttggtggaccaacaaagcagcactgattgccgaagtgtatgaaaatgaaatcgaacaggtacgtaaatttccggatttgggtagctttaaagccgatctggattttctgctgcataatctgtggaaagtttggcgtgaaaccatttgtggtgaagcatttcgttgtgttattgcagaagcacagttggaccctgtaaccctgacccaactgaaagatcagtttatggaacgtcgtcgtgagataccgaaaaaactggttgaagatgccattagcaatggtgaactgccgaaagatatcaatcgtgaactgctgctggatatgatttttggtttttgttggtatcgcctgctgaccgaacagttgaccgttgaacaggatattgaagaatttaccttcctgctgattaatggtgtttgtccgggtacacagtgt

SEQ ID NO: 155 - protein, 200 aa, synthetic construct
MARTPSRSSIGSLRSPHTHKAILTSTIEILKECGYSGLSIESVARRAGAGKPTIYRWWTNKAALIAEVYENEIEQVRKFPDLGSFKADLDFLLHNLWKVWRETICGEAFRCVIAEAQLDPVTLTQLKDQFMERRREIPKKLVEDAISNGELPKDINRELLLDMIFGFCWYRLLTEQLTVEQDIEEFTFLLINGVCPGTQC

SEQ ID NO: 156 - DNA, 198 bp, synthetic construct
  source 1..198: mol_type = other DNA
atggaacaacgcataaccctgaaagattatgcaatgcgctttgggcaaaccaagacagctaaagatctcggcgtatatcaaagcgcgatcaacaaggccattcatgcaggccgaaagatttttttaactataaacgctgatggaagcgtttatgcggaagaggtaaagcccttcccgagtaacaaaaaaacaacagca

SEQ ID NO: 157 - protein, 66 aa, synthetic construct
MEQRITLKDYAMRFGQTKTAKDLGVYQSAINKAIHAGRKIFLTINADGSVYAEEVKPFPSNKKTTA

SEQ ID NO: 158 - DNA, 420 bp, synthetic construct
  source 1..420: mol_type = other DNA
atggaacaacgcataaccctgaaagattatgcaatgcgctttgggcaaaccaagacagctaaagatctcggcgtatatcaaagcgcgatcaacaaggccattcatgcaggccgaaagatttttttaactataaacgctgatggaagcgtttatgcggaagaggtaaagcccttcccgagtaacaaaaaaacaacagcagccggtaccggtggctctggcggcatggaacaacgcataaccctgaaagattatgcaatgcgctttgggcaaaccaagacagctaaagatctcggcgtatatcaaagcgcgatcaacaaggccattcatgcaggccgaaagatttttttaactataaacgctgatggaagcgtttatgcggaagaggtaaagcccttcccgagtaacaaaaaaacaacagca

SEQ ID NO: 159 - protein, 140 aa, synthetic construct
MEQRITLKDYAMRFGQTKTAKDLGVYQSAINKAIHAGRKIFLTINADGSVYAEEVKPFPSNKKTTAAGTGGSGGMEQRITLKDYAMRFGQTKTAKDLGVYQSAINKAIHAGRKIFLTINADGSVYAEEVKPFPSNKKTTA

SEQ ID NO: 160 - protein, 238 aa, synthetic construct
MSKGEELFTGVVPILVELDGDVNGHKFSVSGEGEGDATYGKLTLKFICTTGKLPVPWPTLVTTFSYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYK

SEQ ID NO: 161 - protein, 882 aa, synthetic construct
MNTINIAKNDFSDIELAAIPFNTLADHYGERLAREQLALEHESYEMGEARFRKMFERQLKAGEVADNAAAKPLITTLLPKIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 162 - DNA, 3150 bp, synthetic construct
  source 1..3150: mol_type = other DNA
atgacaattaaagaaatgcctcagccaaaaacgtttggagagcttaaaaatttaccgttattaaacacagataaaccggttcaagctttgatgaaaattgcggatgaattaggagaaatctttaaattcgaggcgcctggtctggtaacgcgcttcttatcaagtcagcgtctaattaaagaagcatgcgatgaatcacgctttgataaaaacttaagtcaagcgcttaaatttgtacgtgattttacaggagacgggttagcgacaagctggacgcacgaaaaaaattggaaaaaagcgcataatatcttacttccaagcttcagtcagcaggcaatgaaaggctatcatgcgatgatggtcgatatcgccgtgcagcttgttcaaaagtgggagcgtctaaatgcagatgagcatattgaagtacccgaagatatgacacgtttaacgcttgatacaattggtctttgcggctttaactatcgctttaacagcttttaccgagatcagcctcatccatttattacaagtatggtccgtgcactggatgaagcaatgaacaagctgcagcgagcaaatccagacgacccagcttatgatgaaaacaagcgccagtttcaagaagatatcaaggtgatgaacgacctagtagataaaattattgcagatcgcaaagcaagcggtgaacaaagcgatgatttattaacgcatatgctaaacggaaaagatccagaaacaggtgagccgcttgatgacgagaacattcgctatcaaattattacattcttaattgcgggacacgaaacaacaagcggtcttttatcatttgcgctgtatttcttagtgaaaaatccacatgtattacaaaaagcagcagaagaagcagcacgagttctagtagatcctgttccaagctacaaacaagtcaaacagcttaaatatgtcggcatggtcttaaacgaagcgctgcgcttatggccaactgctcctgcgttttccctatatgcaaaagaagatacggtgcttggaggagaatatcctttagaaaaaggcgacgaactaatggttctgattcctcagcttcaccgtgataaaacaatttggggagacgatgtggaagagttccgtccagagcgttttgaaaatccaagtgcgattccgcagcatgcgtttaaaccgtttggaaacggtcagcgtgcgtgtccaggtcagcagttcgctcttcatgaagcaacgctggtacttggtatggtgctaaaacactttgactttgaagatcatacaaactacgagctggatattaaagaaactttaacgttaaaacctgaaggctttgtggtaaaagcaaaatcgaaaaaaattccgcttggcggtattccttcacctagccctgaacagtctgctaaaaaagtacgcaaaaaggcagaaaacgctcataatacgccgctgcttgtgctatacggttcaaatatgggaacagctgaaggaacggcgcgtgatttagcagatattgcgatgagcaaaggattcgcaccgcaggtcgctacccttgattcacacgccggaaatcttccgcgcgaaggagctgtattaattgtaacggcgtcttataacggacatccgcctgataacgcaaagcaatttgtcgactggttagaccaagcgtctgctgatgaagtaaaaggcgttcgctactccgtatttggatgcggcgataaaaactgggctactacgtatcaaaaagtgcctgcttttatcgatgaaacgcttgccgctaaaggggcagaaaacatcgctgaccgcggtgaagcagatgcaagcgacgactttgaaggcacatatgaagaatggcgtgaacatatgtggagtgacgtagcagcctactttaacctcgacattgaaaacagtgaagataataaatctactctttcacttcaatttgtcgacagcgccgcggatatgccgcttgcgaaaatgcatggtgcgttttcaacgaacgtcgtagcaagcaaagaacttcaacagccaggcagtgcacgaagcacgcgacaccttgaaattgaacttccaaaagaagcttcttatcaagaaggagatcatttaggtattattcctcgcaactatgaaggaatagtaaaccgtgtaacagcaaggttcggactagatgcatcacagcaaatccgtctggaagcagaagaagaaaaattagctcatttgccactcgctaaaacagtatccgtagaagagcttctgcaatacgtggagcttcaagatcctgttacgcgcacgcagcttcgcgcaatggctgctaaaacggtctgcccgccgcataaagtagagcttgaagccttgcttgaaaagcaagcctacaaagaacaagtgctggcaaaacgtttaacaatgcttgaactgcttgaaaaatacccggcgtgtgaaatggaattcagcgaatttatcgcccttctgccaagcatacgcccgcgttattactcgatttcttcatcacctcgtgtcgatgaaaaacaagcaagcatcacggtcagcgttgtctcaggagaagcgtggagcggatatggagaatataaaggaattgcgtcgaactatcttgctgagctgcaagaaggagatacgattacgtgctttatttccacaccgcagtcagaatttacgctgccaaaagaccctgaaacgccgcttatcatggtcggaccgggaacaggcgtcgcgccgtttagaggcttcgtgcaggctcgcaagcagctaaaagaaaaaggacagtcgcttggagaagcgcatttatacttcggctgccgttcacctcatgaagactatctgtatcaagaagagcttgaaaacgcccaaaatgaaggcatcattacgcttcataccgctttttctcgcgtgccaaatcagccaaaaacatacgttcagcacgtgatggaacaagacggcaagaaattgattgaacttcttgatcaaggagcgcacttctatatttgcggagacggaagccaaatggcacctgacgttgaagcaacgcttatgaaaagctatgctggcgttcaccaagtgagtgaagcagacgctcgcttatggctgcagcagctagaggaaaaaggccgatacgcaaaagacgtgtgggctgggtaa

SEQ ID NO: 163 - protein, 1049 aa, synthetic construct
MTIKEMPQPKTFGELKNLPLLNTDKPVQALMKIADELGEIFKFEAPGLVTRFLSSQRLIKEACDESRFDKNLSQALKFVRDFTGDGLATSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACPGQQFALHEATLVLGMVLKHFDFEDHTNYELDIKETLTLKPEGFVVKAKSKKIPLGGIPSPSPEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGIIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMEFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEKGQSLGEAHLYFGCRSPHEDYLYQEELENAQNEGIITLHTAFSRVPNQPKTYVQHVMEQDGKKLIELLDQGAHFYICGDGSQMAPDVEATLMKSYAGVHQVSEADARLWLQQLEEKGRYAKDVWAG

SEQ ID NO: 164 - DNA, 3150 bp, synthetic construct
  source 1..3150: mol_type = other DNA
atggcaattaaagaaatgcctcagccaaaaacgtttggagagcttaaaaatttaccgttattaaacacagataaaccggttcaagctttgatgaaaattgcggatgaattaggagaaatctttaaattcgaggcgcctggtctggtaacgcgctttttatcaagtcagcgtctaattaaagaagcatgcgatgaatcacgctttgataaaaacttaagtcaagcgcttaaatttgtacgtgattttgcaggagacgggttagcgacaagctggacgcatgaaaaaaattggaaaaaagcgcataatatcttacttccaagcttcagtcagcaggcaatgaaaggctatcatgcgatgatggtcgatatcgccgtgcagcttgttcaaaagtgggagcgtctaaatgcagatgagcatattgaagtaccggaagacatgacacgtttaacgcttgatacaattggtctttgcggctttaactatcgctttaacagcttttaccgagatcagcctcatccatttattacaagtatggtccgtgcactggatgaagcaatgaacaagctgcagcgagcaaatccagacgacccagcttatgatgaaaacaagcgccagtttcaagaagatatcaaggtgatgaacgacctagtagataaaattattgcagatcgcaaagcaagcggtgaacaaagcgatgatttattaacgcatatgctaaacggaaaagatccagaaacgggtgagccgcttgatgacgagaacattcgctatcaaattattacattcttaattgcgggacacgaaacaacaagtggtcttttatcatttgcgctgtatttcttagtgaaaaatccacatgtattacaaaaagcagcagaagaagcagcacgagttctagtagatcctgttccaagctacaaacaagtcaaacagcttaaatatgtcggcatggtcttaaacgaagcgctgcgcttatggccaactctgcctgcgttttccctatatgcaaaagaagatacggtgcttggaggagaatatcctttagaaaaaggcgacgaactaatggttctgattcctcagcttcaccgtgataaaacaatttggggagacgatgtggaagagttccgtccagagcgttttgaaaatccaagtgcgattccgcagcatgcgtttaaaccgtttggaaacggtcagcgtgcgtgtatcggtcagcagttcgctcttcatgaagcaacgctggtacttggtatgatgctaaaacactttgactttgaagatcatacaaactacgagctggatattaaagaaactttaacgttaaaacctgaaggctttgtggtaaaagcaaaatcgaaaaaaattccgcttggcggtattccttcacctagcactgaacagtctgctaaaaaagtacgcaaaaaggcagaaaacgctcataatacgccgctgcttgtgctatacggttcaaatatgggaacagctgaaggaacggcgcgtgatttagcagatattgcaatgagcaaaggatttgcaccgcaggtcgcaacgcttgattcacacgccggaaatcttccgcgcgaaggagctgtattaattgtaacggcgtcttataacggtcatccgcctgataacgcaaagcaatttgtcgactggttagaccaagcgtctgctgatgaagtaaaaggcgttcgctactccgtatttggatgcggcgataaaaactgggctactacgtatcaaaaagtgcctgcttttatcgatgaaacgcttgccgctaaaggggcagaaaacatcgctgaccgcggtgaagcagatgcaagcgacgactttgaaggcacatatgaagaatggcgtgaacatatgtggagtgacgtagcagcctactttaacctcgacattgaaaacagtgaagataataaatctactctttcacttcaatttgtcgacagcgccgcggatatgccgcttgcgaaaatgcacggtgcgttttcaacgaacgtcgtagcaagcaaagaacttcaacagccaggcagtgcacgaagcacgcgacatcttgaaattgaacttccaaaagaagcttcttatcaagaaggagatcatttaggtgttattcctcgcaactatgaaggaatagtaaaccgtgtaacagcaaggttcggcctagatgcatcacagcaaatccgtctggaagcagaagaagaaaaattagctcatttgccactcgctaaaacagtatccgtagaagagcttctgcaatacgtggagcttcaagatcctgttacgcgcacgcagcttcgcgcaatggctgctaaaacggtctgcccgccgcataaagtagagcttgaagccttgcttgaaaagcaagcctacaaagaacaagtgctggcaaaacgtttaacaatgcttgaactgcttgaaaaatacccggcgtgtgaaatgaaattcagcgaatttatcgcccttctgccaagcatacgcccgcgctattactcgatttcttcatcacctcgtgtcgatgaaaaacaagcaagcatcacggtcagcgttgtctcaggagaagcgtggagcggatatggagaatataaaggaattgcgtcgaactatcttgccgagctgcaagaaggagatacgattacgtgctttatttccacaccgcagtcagaatttacgctgccaaaagaccctgaaacgccgcttatcatggtcggaccgggaacaggcgtcgcgccgtttagaggctttgtgcaggcgcgcaaacagctaaaagaacaaggacagtcacttggagaagcacatttatacttcggctgccgttcacctcatgaagactatctgtatcaagaagagcttgaaaacgcccaaagcgaaggcatcattacgcttcataccgctttttctcgcatgccaaatcagccgaaaacatacgttcagcacgtaatggaacaagacggcaagaaattgattgaacttcttgatcaaggagcgcacttctatatttgcggagacggaagccaaatggcacctgccgttgaagcaacgcttatgaaaagctatgctgacgttcaccaagtgagtgaagcagacgctcgcttatggctgcagcagctagaagaaaaaggccgatacgcaaaagacgtgtgggctgggtaa

SEQ ID NO: 165 - protein, 1049 aa, synthetic construct
MAIKEMPQPKTFGELKNLPLLNTDKPVQALMKIADELGEIFKFEAPGLVTRFLSSQRLIKEACDESRFDKNLSQALKFVRDFAGDGLATSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTLPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 166 - DNA, 1458 bp, synthetic construct
  source 1..1458: mol_type = other DNA
gttgccttgttggtctgtctgactgtcatggtgttaatgagtgtgtggcaacaacggaagagcaaaggcaagttaccgcccggcccaactccgctgccctttataggcaactatcttcagttgaacacagagcagatgtataactccttgatgaagatctcggaacgttacggtccagtctttacgatacatttgggccctcggcgggttgttgtattgtgtggacatgacgctgtgcgcgaggcacttgtagaccaagcggaagaattcagtgggcgcggtgaacaagcgacattcgactgggtcttcaagggatatggagttgttttctctaatggagaacgggcaaaacagcttcgtcggtttagtatagcgacacttcgggatttcggagtggggaaaagaggcattgaagaacgcattcaagaggaagcgggatttctgatagacgctcttagagggacaggcggtgcaaatatcgaccccacgttttttttgagccgtactgttagcaatgtcattagcagcatcgtgtttggtgatcggttcgattacaaggacaaagaatttctgtcgttgttgagaatgatgttagggatcttccaatttacttcgacgtcgactgggcagttgtacgagatgttttcgtcggtaatgaaacatttgccgggtccccagcagcaagcattccagctgttacaaggattagaagattttatagctaagaaagtagagcataatcaacggacgttagacccaaactcaccaagagatttcatagacagcttcttgatacggatgcaggaggaggagaaaaacccaaatacagaattttatcttaagaatctggttatgactacacttaatttgtttatagggggtacagagacagtgtcgaccacgttgcgttacggtttcctgctgctgatgaaacaccccgaagttgaagctaaagtccatgaagagatagaccgggttatcggaaaaaacagacaacctaaattcgaggatcgcgcaaagatgccttacatggaagctgtaatacatgaaatacaaagattcggtgatgttatcccgatgtctttagcgcgccgtgtcaaaaaggataccaagttccgcgacttcttcctgccaaaaggaacagaggtgtatcccatgttggggtctgtcttgagagatccgtcatttttcagcaatccccaagattttaacccgcagcattttttgaatgagaaagggcaattcaaaaagtcagacgctttcgtgcctttctcaataggcaaacggaactgctttggagaggggctggcccggatggaacttttcttgttcttcacaacggtcatgcaaaattttcgcctgaaatcatcccaatcacctaaggatatcgatgtctcgcccaaacacgtcgggtttgccaccatcccccggaactacaccatgtcgtttcttcctcgg

SEQ ID NO: 167 - protein, 486 aa, synthetic construct
VALLVCLTVMVLMSVWQQRKSKGKLPPGPTPLPFIGNYLQLNTEQMYNSLMKISERYGPVFTIHLGPRRVVVLCGHDAVREALVDQAEEFSGRGEQATFDWVFKGYGVVFSNGERAKQLRRFSIATLRDFGVGKRGIEERIQEEAGFLIDALRGTGGANIDPTFFLSRTVSNVISSIVFGDRFDYKDKEFLSLLRMMLGIFQFTSTSTGQLYEMFSSVMKHLPGPQQQAFQLLQGLEDFIAKKVEHNQRTLDPNSPRDFIDSFLIRMQEEEKNPNTEFYLKNLVMTTLNLFIGGTETVSTTLRYGFLLLMKHPEVEAKVHEEIDRVIGKNRQPKFEDRAKMPYMEAVIHEIQRFGDVIPMSLARRVKKDTKFRDFFLPKGTEVYPMLGSVLRDPSFFSNPQDFNPQHFLNEKGQFKKSDAFVPFSIGKRNCFGEGLARMELFLFFTTVMQNFRLKSSQSPKDIDVSPKHVGFATIPRNYTMSFLPR

SEQ ID NO: 168 - DNA, 1296 bp, synthetic construct
  source 1..1296: mol_type = other DNA
atgggaacaacacgcatggataccttcaatccccaagaaagccgtcttgcaacgaactttgatgaagcagtgcgtgccaaggtcgagcgccccgctaatgtacctgaggaccgcgtatatgaaattgacatgtatgcactgaacgggattgaagatggatatcacgaagcgtggaaaaaggtgcaacatccaggcatccccgatcttatttggacgccatttacaggggggcactggattgcgacgaatggtgatacggttaaagaagtgtatagtgatccgacccgcttcagctccgaagtcatctttttacccaaggaggccggtgaaaaataccagatggtccccaccaagatggaccctcctgaacacacaccatatcgcaaggccctggacaagggattgaaccttgccaagattcgcaaggttgaggacaaggtccgcgaagttgcctctagtctgatcgattcattcgccgcccgcggagagtgtgacttcgctgccgaatatgctgagttatttcctgttcatgtctttatggcgctggctgacctgcctctggaggacatcccggttcttagcgaatacgcccgtcaaatgacccgccctgaaggtaatacgccagaggaaatggctacggatttagaggcaggcaataatggtttttatgcatatgtcgaccctatcatccgcgcccgtgtggggggagacggagatgatcttatcaccttgatggttaatagtgagattaacggtgagcgcatcgcgcatgacaaagctcaaggccttatctcgttgctgttattgggaggcctggatacggtcgtcaatttcctgtccttctttatgattcaccttgcacgccatcccgagctggtcgcggaacttcgttcggacccactgaaacttatgcgcggcgccgaagagatgtttcgccgttttccggtagtcagtgaagcccgtatggtggcaaaggaccaggagtataagggggtctttttgaagcgtggcgatatgattttattacctaccgctttacacggtctggacgatgccgctaacccagaaccgtggaaattagacttttcacgccgctcaattagccattcaacttttggaggggggccacatcgctgtgcaggtatgcacttagcccgtatggaggtaatcgttacactggaggagtggcttaaacgtattcccgaattttctttcaaagagggggaaaccccaatctatcactctggaatcgtagcagctgtcgaaaacgtccccttggtgtggccgatcgcacgt

SEQ ID NO: 169 - protein, 432 aa, synthetic construct
MGTTRMDTFNPQESRLATNFDEAVRAKVERPANVPEDRVYEIDMYALNGIEDGYHEAWKKVQHPGIPDLIWTPFTGGHWIATNGDTVKEVYSDPTRFSSEVIFLPKEAGEKYQMVPTKMDPPEHTPYRKALDKGLNLAKIRKVEDKVREVASSLIDSFAARGECDFAAEYAELFPVHVFMALADLPLEDIPVLSEYARQMTRPEGNTPEEMATDLEAGNNGFYAYVDPIIRARVGGDGDDLITLMVNSEINGERIAHDKAQGLISLLLLGGLDTVVNFLSFFMIHLARHPELVAELRSDPLKLMRGAEEMFRRFPVVSEARMVAKDQEYKGVFLKRGDMILLPTALHGLDDAANPEPWKLDFSRRSISHSTFGGGPHRCAGMHLARMEVIVTLEEWLKRIPEFSFKEGETPIYHSGIVAAVENVPLVWPIAR

SEQ ID NO: 170 - DNA, 43 bp, synthetic construct
  source 1..43: mol_type = other DNA
tactggccgaagccatggcagctgcggaaccgcagtatgaaga

SEQ ID NO: 171 - DNA, 49 bp, synthetic construct
  source 1..49: mol_type = other DNA
tgccatggcttcggccagtacgcgagctttacgacgaccttcagcaata

SEQ ID NO: 172 - DNA, 48 bp, synthetic construct
  source 1..48: mol_type = other DNA
tacaatcagggttccgtgcagctgcggaaccgcagtatgaagaaattc

SEQ ID NO: 173 - DNA, 49 bp, synthetic construct
  source 1..49: mol_type = other DNA
agctgcacggaaccctgattgtaaaactgcacgacgaccttcagcaata

SEQ ID NO: 174 - DNA, 49 bp, synthetic construct
  source 1..49: mol_type = other DNA
tggcagctgcagctgcggaaccgcagtatgaagaaattccgatttatct

SEQ ID NO: 175 - DNA, 50 bp, synthetic construct
  source 1..50: mol_type = other DNA
tggcttcggccagtacgcgagctttagctgcacgacgaccttcagcaata

SEQ ID NO: 176 - DNA, 39 bp, synthetic construct
  source 1..39: mol_type = other DNA
tcgcgtactggccgaagccatggcagctgcagctgcgga

SEQ ID NO: 177 - DNA, 42 bp, synthetic construct
  source 1..42: mol_type = other DNA
gttccgcagctgcagctgccatggcttcggccagtacgcgag

SEQ ID NO: 178 - DNA, 46 bp, synthetic construct
  source 1..46: mol_type = other DNA
cagctgcagctgcggaaccgcagtatgaagaaattccgatttatct

SEQ ID NO: 179 - DNA, 50 bp, synthetic construct
  source 1..50: mol_type = other DNA
cacggaaccctgattgtaaaactgcagctgcacgacgaccttcagcaata

SEQ ID NO: 180 - DNA, 50 bp, synthetic construct
  source 1..50: mol_type = other DNA
tgcagttttacaatcagggttccgtgcagctgcagctgcggaaccgcagt

SEQ ID NO: 181 - DNA, 50 bp, synthetic construct
  source 1..50: mol_type = other DNA
actgcggttccgcagctgcagctgcacggaaccctgattgtaaaactgca

SEQ ID NO: 182 - DNA, 44 bp, synthetic construct
  source 1..44: mol_type = other DNA
atggcagctgcagctgcagctgcggaaccgcagtatgaagaaat

SEQ ID NO: 183 - DNA, 50 bp, synthetic construct
  source 1..50: mol_type = other DNA
cggccagtacgcgagctttagctgcagctgcacgacgaccttcagcaata

SEQ ID NO: 184 - DNA, 49 bp, synthetic construct
  source 1..49: mol_type = other DNA
tgcagctaaagctcgcgtactggccgaagccatggcagctgcagctgca

SEQ ID NO: 185 - DNA, 49 bp, synthetic construct
  source 1..49: mol_type = other DNA
tgcagctgcagctgccatggcttcggccagtacgcgagctttagctgca

SEQ ID NO: 186 - DNA, 48 bp, synthetic construct
  source 1..48: mol_type = other DNA
gttccgtgcagctgcagctgcagctgcggaaccgcagtatgaagaaat

SEQ ID NO: 187 - DNA, 50 bp, synthetic construct
  source 1..50: mol_type = other DNA
accctgattgtaaaactgcagctgcagctgcacgacgaccttcagcaata

SEQ ID NO: 188 - DNA, 40 bp, synthetic construct
  source 1..40: mol_type = other DNA
agctgcagttttacaatcagggttccgtgcagctgcagct

SEQ ID NO: 189 - DNA, 40 bp, synthetic construct
  source 1..40: mol_type = other DNA
agctgcagctgcacggaaccctgattgtaaaactgcagct

SEQ ID NO: 190 - DNA, 47 bp, synthetic construct
  source 1..47: mol_type = other DNA
gcttaaaaaaccagggcgctacaagctatatgaattcgctgttacag

SEQ ID NO: 191 - DNA, 22 bp, synthetic construct
  source 1..22: mol_type = other DNA
gtagcgccctggttttttaagc

SEQ ID NO: 192 - DNA, 60 bp, synthetic construct
  source 1..60: mol_type = other DNA
ctgacaagcattaaatgggctgataataatagctatctggctacagcattattaacattg

SEQ ID NO: 193 - DNA, 22 bp, synthetic construct
  source 1..22: mol_type = other DNA
cagcccatttaatgcttgtcag

SEQ ID NO: 194 - DNA, 28 bp, synthetic construct
  source 1..28: mol_type = other DNA
atatggtctcacatggcggatcgccagg

SEQ ID NO: 195 - DNA, 33 bp, synthetic construct
  source 1..33: mol_type = other DNA
atatggtctcatttaaaagttcagggtgcagcc

SEQ ID NO: 196 - DNA, 46 bp, synthetic construct
  source 1..46: mol_type = other DNA
tcgagctcttaaagaggagaaaggtcatgtcggggttccgtaaaat

SEQ ID NO: 197 - DNA, 46 bp, synthetic construct
  source 1..46: mol_type = other DNA
atccgccaaaacagccaagcttttattggaaagttacgccgctaca

SEQ ID NO: 198 - DNA, 45 bp, synthetic construct
  source 1..45: mol_type = other DNA
agctcttaaagaggagaaaggtcatgagtaaaaagcatacagggt

SEQ ID NO: 199 - DNA, 44 bp, synthetic construct
  source 1..44: mol_type = other DNA
caatgatgatgatgatgatggttgtgaattttgatagccttttc

SEQ ID NO: 200 - DNA, 46 bp, synthetic construct
  source 1..46: mol_type = other DNA
agctcttaaagaggagaaaggtcatggaggttcgtactattaaggt

SEQ ID NO: 201 - DNA, 47 bp, synthetic construct
  source 1..47: mol_type = other DNA
tccgccaaaacagccaagcttttacttgatcgtcgtggtgtaagagt

SEQ ID NO: 202 - DNA, 35 bp, synthetic construct
  source 1..35: mol_type = other DNA
ggaaccgcagtatgaagaaattccgatttatctgt

SEQ ID NO: 203 - DNA, 58 bp, synthetic construct
  source 1..58: mol_type = other DNA
tcttcatactgcggttccgcagctgcccccccacgtaaacgacgaccttcagcaatag

SEQ ID NO: 204 - DNA, 46 bp, synthetic construct
  source 1..46: mol_type = other DNA
cattacgtggtggagcagcagcagcagcagctgcggaaccgcagta

SEQ ID NO: 205 - DNA, 45 bp, synthetic construct
  source 1..45: mol_type = other DNA
tgctccaccacgtaatgctgctgctgcacgacgaccttcagcaat

SEQ ID NO: 206 - DNA, 74 bp, synthetic construct
  source 1..74: mol_type = other DNA
cgatgagaaggatgtcagtgtgtaatcgacagcagcggataaggaggtattaatgtcggggttccgtaaaatgg

SEQ ID NO: 207 - DNA, 50 bp, synthetic construct
  source 1..50: mol_type = other DNA
tttttttagggccctactgactgttattggaaagttacgccgctacattg

SEQ ID NO: 208 - DNA, 49 bp, synthetic construct
  source 1..49: mol_type = other DNA
acagtaacataggggaggattaatatggcggatcgccagggcaccgtga

SEQ ID NO: 209 - DNA, 49 bp, synthetic construct
  source 1..49: mol_type = other DNA
attaatcctcccctatgttactgtggatattacacactgacatccttct

SEQ ID NO: 210 - DNA, 49 bp, synthetic construct
  source 1..49: mol_type = other DNA
catatcctaagcgcttcttaaatgagtaaaaagcatacagggtacgtgg

SEQ ID NO: 211 - DNA, 56 bp, synthetic construct
  source 1..56: mol_type = other DNA
gtttttttttagggccctactgactgttagttgtgaattttgatagccttttccag

SEQ ID NO: 212 - DNA, 74 bp, synthetic construct
  source 1..74: mol_type = other DNA
cgatgagaaggatgtcagtgtgtaaatktgcsamcmttaaaggagsttccaaatggaggttcgtactattaagg

SEQ ID NO: 213 - DNA, 40 bp, synthetic construct
  source 1..40: mol_type = other DNA
tagggccctactgactgttacttgatcgtcgtggtgtaag

SEQ ID NO: 214 - protein, 8 aa, synthetic construct
FHLQGPED

SEQ ID NO: 215 - protein, 8 aa, synthetic construct
FELQGPED

SEQ ID NO: 216 - protein, 8 aa, synthetic construct
AVLQSGFR

SEQ ID NO: 217 - protein, 8 aa, synthetic construct
GVLQSGLV

SEQ ID NO: 218 - protein, 8 aa, synthetic construct
AGRRSVSG

SEQ ID NO: 219 - protein, 8 aa, synthetic construct
AGRKSLTL

SEQ ID NO: 220 - protein, 8 aa, synthetic construct
AGRKSIAL

SEQ ID NO: 221 - protein, 8 aa, synthetic construct
SGRKSITL

SEQ ID NO: 222 - protein, 8 aa, synthetic construct
SGKRSQIG

SEQ ID NO: 223 - protein, 8 aa, synthetic construct
AGKRSAVS

SEQ ID NO: 224 - protein, 8 aa, synthetic construct
HSKRGGAL

SEQ ID NO: 225 - protein, 8 aa, synthetic construct
EGRRGAAE

SEQ ID NO: 226 - protein, 8 aa, synthetic construct
AGKRGAAF

SEQ ID NO: 227 - protein, 8 aa, synthetic construct
LRTQSFSN

SEQ ID NO: 228 - protein, 8 aa, synthetic construct
AKVQGPGF

SEQ ID NO: 229 - protein, 8 aa, synthetic construct
ATVQGPSL

SEQ ID NO: 230 - protein, 8 aa, synthetic construct
YTAGNKVD

SEQ ID NO: 231 - protein, 8 aa, synthetic construct
YIAGNKID

SEQ ID NO: 232 - protein, 8 aa, synthetic construct
EAGAGSVE

SEQ ID NO: 233 - protein, 8 aa, synthetic construct
RAGAGIIE

SEQ ID NO: 234 - DNA, 24 bp, synthetic construct
  source 1..24: mol_type = other DNA
gcagttttacaatcagggttccgt

SEQ ID NO: 235 - protein, 435 aa, synthetic construct
MEMEKEFEQIDKSGSWAAIYQDIRHEASDFPCRVAKLPKNKNRNRYRDVSPFDHSRIKLHQEDNDYINASLIKMEEAQRSYILTQGPLPNTCGHFWEMVWEQKSRGVVMLNRVMEKGSLKCAQYWPQKEEKEMIFEDTNLKLTLISEDIKSYYTVRQLELENLTTQETREILHFHYTTWPDFGVPESPASFLNFLFKVRESGSLSPEHGPVVVHCSAGIGRSGTFCLADTCLLLMDKRKDPSSVDIKKVLLEMRKFRMGLIQTADQLRFSYLAVIEGAKFIMGDSSVQDQWKELSHEDLEPPPEHIPPPPRPPKRILEPHNGKCREFFPNHQWVKEETQEDKDCPIKEEKGSPLNAAPYGIESMSQDTEVRSRVVGGSLRGAQAASPAKGEPSLPEKDEDHALSYWKPFLVNMCVATVLTAGAYLCYRFLFNSNT

SEQ ID NO: 236 - protein, 435 aa, synthetic construct; identical to SEQ ID NO: 235

SEQ ID NO: 237 - protein, 415 aa, synthetic construct
MPTTIEREFEELDTQRRWQPLYLEIRNESHDYPHRVAKFPENRNRNRYRDVSPYDHSRVKLQNAENDYINASLVDIEEAQRSYILTQGPLPNTCCHFWLMVWQQKTKAVVMLNRIVEKESVKCAQYWPTDDQEMLFKETGFSVKLLSEDVKSYYTVHLLQLENINSGETRTISHFHYTTWPDFGVPESPASFLNFLFKVRESGSLNPDHGPAVIHCSAGIGRSGTFSLVDTCLVLMEKGDDINIKQVLLNMRKYRMGLIQTPDQLRFSYMAIIEGAKCIKGDSSIQKRWKELSKEDLSPAFDHSPNKIMTEKYNGNRIGLEEEKLTGDRCTGLSSKMQDTMEENSESALRKRIREDRKATTAQKVQQMKQRLNENERKRKRWLYWQPILTKMGFMSVILVGAFVGWTLFFQQNAL

SEQ ID NO: 238 - protein, 415 aa, synthetic construct; identical to SEQ ID NO: 237

SEQ ID NO: 239 - protein, 1997 aa, synthetic construct
MLSHGAGLALWITLSLLQTGLAEPERCNFTLAESKASSHSVSIQWRILGSPCNFSLIYSSDTLGAALCPTFRIDNTTYGCNLQDLQAGTIYNFRIISLDEERTVVLQTDPLPPARFGVSKEKTTSTSLHVWWTPSSGKVTSYEVQLFDENNQKIQGVQIQESTSWNEYTFFNLTAGSKYNIAITAVSGGKRSFSVYTNGSTVPSPVKDIGISTKANSLLISWSHGSGNVERYRLMLMDKGILVHGGVVDKHATSYAFHGLTPGYLYNLTVMTEAAGLQNYRWKLVRTAPMEVSNLKVTNDGSLTSLKVKWQRPPGNVDSYNITLSHKGTIKESRVLAPWITETHFKELVPGRLYQVTVSCVSGELSAQKMAVGRTFPDKVANLEANNNGRMRSLVVSWSPPAGDWEQYRILLFNDSVVLLNITVGKEETQYVMDDTGLVPGRQYEVEVIVESGNLKNSERCQGRTVPLAVLQLRVKHANETSLSIMWQTPVAEWEKYIISLADRDLLLIHKSLSKDAKEFTFTDLVPGRKYMATVTSISGDLKNSSSVKGRTVPAQVTDLHVANQGMTSSLFTNWTQAQGDVEFYQVLLIHENVVIKNESISSETSRYSFHSLKSGSLYSVVVTTVSGGISSRQVVVEGRTVPSSVSGVTVNNSGRNDYLSVSWLLAPGDVDNYEVTLSHDGKVVQSLVIAKSVRECSFSSLTPGRLYTVTITTRSGKYENHSFSQERTVPDKVQGVSVSNSARSDYLRVSWVHATGDFDHYEVTIKNKNNFIQTKSIPKSENECVFVQLVPGRLYSVTVTTKSGQYEANEQGNGRTIPEPVKDLTLRNRSTEDLHVTWSGANGDVDQYEIQLLFNDMKVFPPFHLVNTATEYRFTSLTPGRQYKILVLTISGDVQQSAFIEGFTVPSAVKNIHISPNGATDSLTVNWTPGGGDVDSYTVSAFRHSQKVDSQTIPKHVFEHTFHRLEAGEQYQIMIASVSGSLKNQINVVGRTVPASVQGVIADNAYSSYSLIVSWQKAAGVAERYDILLLTENGILLRNTSEPATTKQHKFEDLTPGKKYKIQILTVSGGLFSKEAQTEGRTVPAAVTDLRITENSTRHLSFRWTASEGELSWYNIFLYNPDGNLQERAQVDPLVQSFSFQNLLQGRMYKMVIVTHSGELSNESFIFGRTVPASVSHLRGSNRNTTDSLWFNWSPASGDFDFYELILYNPNGTKKENWKDKDLTEWRFQGLVPGRKYVLWVVTHSGDLSNKVTAESRTAPSPPSLMSFADIANTSLAITWKGPPDWTDYNDFELQWLPRDALTVFNPYNNRKSEGRIVYGLRPGRSYQFNVKTVSGDSWKTYSKPIFGSVRTKPDKIQNLHCRPQNSTAIACSWIPPDSDFDGYSIECRKMDTQEVEFSRKLEKEKSLLNIMMLVPHKRYLVSIKVQSAGMTSEVVEDSTITMIDRPPPPPPHIRVNEKDVLISKSSINFTVNCSWFSDTNGAVKYFTVVVREADGSDELKPEQQHPLPSYLEYRHNASIRVYQTNYFASKCAENPNSNSKSFNIKLGAEMESLGGKCDPTQQKFCDGPLKPHTAYRISIRAFTQLFDEDLKEFTKPLYSDTFFSLPITTESEPLFGAIEGVSAGLFLIGMLVAVVALLICRQKVSHGRERPSARLSIRRDRPLSVHLNLGQKGNRKTSCPIKINQFEGHFMKLQADSNYLLSKEYEELKDVGRNQSCDIALLPENRGKNRYNNILPYDATRVKLSNVDDDPCSDYINASYIPGNNFRREYIVTQGPLPGTKDDFWKMVWEQNVHNIVMVTQCVEKGRVKCDHYWPADQDSLYYGDLILQMLSESVLPEWTIREFKICGEEQLDAHRLIRHFHYTVWPDHGVPETTQSLIQFVRTVRDYINRSPGAGPTVVHCSAGVGRTGTFIALDRILQQLDSKDSVDIYGAVHDLRLHRVHMVQTECQYVYLHQCVRDVLRARKLRSEQENPLFPIYENVNPEYHRDPVYSRH

SEQ ID NO: 240 - protein, 1306 aa, synthetic construct
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 241 - protein, 595 aa, synthetic construct
MVRWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLNCSDPTSERWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYYATRVNAADIENRVLELNKKQESEDTAKAGFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQFIETTKKKLEVLQSQKGQESEYGNITYPPAMKNAHAKASRTSSKHKEDVYENLHTKNKREEKVKKQRSADKEKSKGSLKRK

SEQ ID NO: 242 - protein, 807 aa, synthetic construct
MDQREILQKFLDEAQSKKITKEEFANEFLKLKRQSTKYKADKTYPTTVAEKPKNIKKNRYKDILPYDYSRVELSLITSDEDSSYINANFIKGVYGPKAYIATQGPLSTTLLDFWRMIWEYSVLIIVMACMEYEMGKKKCERYWAEPGEMQLEFGPFSVSCEAEKRKSDYIIRTLKVKFNSETRTIYQFHYKNWPDHDVPSSIDPILELIWDVRCYQEDDSVPICIHCSAGCGRTGVICAIDYTWMLLKDGIIPENFSVFSLIREMRTQRPSLVQTQEQYELVYNAVLELFKRQMDVIRDKHSGTESQAKHCIPEKNHTLQADSYSPNLPKSTTKAAKMMNQQRTKMEIKESSSFDFRTSEISAKEELVLHPAKSSTSFDFLELNYSFDKNADTTMKWQTKAFPIVGEPLQKHQSLDLGSLLFEGCSNSKPVNAAGRYFNSKVPITRTKSTPFELIQQRETKEVDSKENFSYLESQPHDSCFVEMQAQKVMHVSSAELNYSLPYDSKHQIRNASNVKHHDSSALGVYSYIPLVENPYFSSWPPSGTSSKMSLDLPEKQDGTVFPSSLLPTSSTSLFSYYNSHDSLSLNSPTNISSLLNQESAVLATAPRIDDEIPPPLPVRTPESFIVVEEAGEFSPNVPKSLSSAVKVKIGTSLEWGGTSEPKKFDDSVILRPSKSVKLRSPKSELHQDRSSPPPPLPERTLESFFLADEDCMQAQSIETYSTSYPDTMENSTSSKQTLKTPGKSFTRSKSLKILRNMKKSICNSCPPNKPAESVQSNNSSSFLNFGFANRFSKPKGPRNPPPTWNI

SEQ ID NO: 243 - protein, 1948 aa, synthetic construct
MAPTWGPGMVSVVGPMGLLVVLLVGGCAAEEPPRFIKEPKDQIGVSGGVASFVCQATGDPKPRVTWNKKGKKVNSQRFETIEFDESAGAVLRIQPLRTPRDENVYECVAQNSVGEITVHAKLTVLREDQLPSGFPNIDMGPQLKVVERTRTATMLCAASGNPDPEITWFKDFLPVDPSASNGRIKQLRSETFESTPIRGALQIESSEETDQGKYECVATNSAGVRYSSPANLYVRELREVRRVAPRFSILPMSHEIMPGGNVNITCVAVGSPMPYVKWMQGAEDLTPEDDMPVGRNVLELTDVKDSANYTCVAMSSLGVIEAVAQITVKSLPKAPGTPMVTENTATSITITWDSGNPDPVSYYVIEYKSKSQDGPYQIKEDITTTRYSIGGLSPNSEYEIWVSAVNSIGQGPPSESVVTRTGEQAPASAPRNVQARMLSATTMIVQWEEPVEPNGLIRGYRVYYTMEPEHPVGNWQKHNVDDSLLTTVGSLLEDETYTVRVLAFTSVGDGPLSDPIQVKTQQGVPGQPMNLRAEARSETSITLSWSPPRQESIIKYELLFREGDHGREVGRTFDPTTSYVVEDLKPNTEYAFRLAARSPQGLGAFTPVVRQRTLQSKPSAPPQDVKCVSVRSTAILVSWRPPPPETHNGALVGYSVRYRPLGSEDPEPKEVNGIPPTTTQILLEALEKWTQYRITTVAHTEVGPGPESSPVVVRTDEDVPSAPPRKVEAEALNATAIRVLWRSPAPGRQHGQIRGYQVHYVRMEGAEARGPPRIKDVMLADAQWETDDTAEYEMVITNLQPETAYSITVAAYTMKGDGARSKPKVVVTKGAVLGRPTLSVQQTPEGSLLARWEPPAGTAEDQVLGYRLQFGREDSTPLATLEFPPSEDRYTASGVHKGATYVFRLAARSRGGLGEEAAEVLSIPEDTPRGHPQILEAAGNASAGTVLLRWLPPVPAERNGAIVKYTVAVREAGALGPARETELPAAAEPGAENALTLQGLKPDTAYDLQVRAHTRRGPGPFSPPVRYRTFLRDQVSPKNFKVKMIMKTSVLLSWEFPDNYNSPTPYKIQYNGLTLDVDGRTTKKLITHLKPHTFYNFVLTNRGSSLGGLQQTVTAWTAFNLLNGKPSVAPKPDADGFIMVYLPDGQSPVPVQSYFIVMVPLRKSRGGQFLTPLGSPEDMDLEELIQDISRLQRRSLRHSRQLEVPRPYIAARFSVLPPTFHPGDQKQYGGFDNRGLEPGHRYVLFVLAVLQKSEPTFAASPFSDPFQLDNPDPQPIVDGEEGLIWVIGPVLAVVFIICIVIAILLYKNKPDSKRKDSEPRTKCLLNNADLAPHHPKDPVEMRRINFQTPDSGLRSPLREPGFHFESMLSHPPIPIADMAEHTERLKANDSLKLSQEYESIDPGQQFTWEHSNLEVNKPKNRYANVIAYDHSRVILQPIEGIMGSDYINANYVDGYRCQNAYIATQGPLPETFGDFWRMVWEQRSATIVMMTRLEEKSRIKCDQYWPNRGTETYGFIQVTLLDTIELATFCVRTFSLHKNGSSEKREVRQFQFTAWPDHGVPEYPTPFLAFLRRVKTCNPPDAGPIVVHCSAGVGRTGCFIVIDAMLERIKPEKTVDVYGHVTLMRSQRNYMVQTEDQYSFIHEALLEAVGCGNTEVPARSLYAYIQKLAQVEPGEHVTGMELEFKRLANSKAHTSRFISANLPCNKFKNRLVNIMPYESTRVCLQPIRGVEGSDYINASFIDGYRQQKAYIATQGPLAETTEDFWRMLWENNSTIVVMLTKLREMGREKCHQYWPAERSARYQYFVVDPMAEYNMPQYILREFKVTDARDGQSRTVRQFQFTDWPEQGVPKSGEGFIDFIGQVHKTKEQFGQDGPISVHCSAGVGRTGVFITLSIVLERMRYEGVVDIFQTVKMLRTQRPAMVQTEDEYQFCYQAALEYLGSFDHYAT

SEQ ID NO: 244 - protein, 1452 aa, synthetic construct
MRGLGTCLATLAGLLLTAAGETFSGGCLFDEPYSTCGYSQSEGDDFNWEQVNTLTKPTSDPWMPSGSFMLVNASGRPEGQRAHLLLPQLKENDTHCIDFHYFVSSKSNSPPGLLNVYVKVNNGPLGNPIWNISGDPTRTWNRAELAISTFWPNFYQVIFEVITSGHQGYLAIDEVKVLGHPCTRTPHFLRIQNVEVNAGQFATFQCSAIGRTVAGDRLWLQGIDVRDAPLKEIKVTSSRRFIASFNVVNTTKRDAGKYRCMIRTEGGVGISNYAELVVKEPPVPIAPPQLASVGATYLWIQLNANSINGDGPIVAREVEYCTASGSWNDRQPVDSTSYKIGHLDPDTEYEISVLLTRPGEGGTGSPGPALRTRTKCADPMRGPRKLEVVEVKSRQITIRWEPFGYNVTRCHSYNLTVHYCYQVGGQEQVREEVSWDTENSHPQHTITNLSPYTNVSVKLILMNPEGRKESQELIVQTDEDLPGAVPTESIQGSTFEEKIFLQWREPTQTYGVITLYEITYKAVSSFDPEIDLSNQSGRVSKLGNETHFLFFGLYPGTTYSFTIRASTAKGFGPPATNQFTTKISAPSMPAYELETPLNQTDNTVTVMLKPAHSRGAPVSVYQIVVEEERPRRTKKTTEILKCYPVPIHFQNASLLNSQYYFAAEFPADSLQAAQPFTIGDNKTYNGYWNTPLLPYKSYRIYFQAASRANGETKIDCVQVATKGAATPKPVPEPEKQTDHTVKIAGVIAGILLFVIIFLGVVLVMKKRKLAKKRKETMSSTRQEMTVMVNSMDKSYAEQGTNCDEAFSFMDTHNLNGRSVSSPSSFTMKTNTLSTSVPNSYYPDETHTMASDTSSLVQSHTYKKREPADVPYQTGQLHPAIRVADLLQHITQMKCAEGYGFKEEYESFFEGQSAPWDSAKKDENRMKNRYGNIIAYDHSRVRLQTIEGDTNSDYINGNYIDGYHRPNHYIATQGPMQETIYDFWRMVWHENTASIIMVTNLVEVGRVKCCKYWPDDTEIYKDIKVTLIETELLAEYVIRTFAVEKRGVHEIREIRQFHFTGWPDHGVPYHATGLLGFVRQVKSKSPPSAGPLVVHCSAGAGRTGCFIVIDIMLDMAEREGVVDIYNCVRELRSRRVNMVQTEEQYVFIHDAILEACLCGDTSVPASQVRSLYYDMNKLDPQTNSSQIKEEFRTLNMVTPTLRVEDCSIALLPRNHEKNRCMDILPPDRCLPFLITIDGESSNYINAALMDSYKQPSAFIVTQHPLPNTVKDFWRLVLDYHCTSVVMLNDVDPAQLCPQYWPENGVHRHGPIQVEFVSADLEEDIISRIFRIYNAARPQDGYRMVQQFQFLGWPMYRDTPVSKRSFLKLIRQVDKWQEEYNGGEGRTVVHCLNGGGRSGTFCAISIVCEMLRHQRTVDVFHAVKTLRNNKPNMVDLLDQYKFCYEVALEYLNSG

SEQ ID NO: 245 - protein, 2316 aa, synthetic construct
MRILKRFLACIQLLCVCRLDWANGYYRQQRKLVEEIGWSYTGALNQKNWGKKYPTCNSPKQSPINIDEDLTQVNVNLKKLKFQGWDKTSLENTFIHNTGKTVEINLTNDYRVSGGVSEMVFKASKITFHWGKCNMSSDGSEHSLEGQKFPLEMQIYCFDADRFSSFEEAVKGKGKLRALSILFEVGTEENLDFKAIIDGVESVSRFGKQAALDPFILLNLLPNSTDKYYIYNGSLTSPPCTDTVDWIVFKDTVSISESQLAVFCEVLTMQQSGYVMLMDYLQNNFREQQYKFSRQVFSSYTGKEEIHEAVCSSEPENVQADPENYTSLLVTWERPRVVYDTMIEKFAVLYQQLDGEDQTKHEFLTDGYQDLGAILNNLLPNMSYVLQIVAICTNGLYGKYSDQLIVDMPTDNPELDLFPELIGTEEIIKEEEEGKDIEEGAIVNPGRDSATNQIRKKEPQISTTTHYNRIGTKYNEAKTNRSPTRGSEFSGKGDVPNTSLNSTSQPVTKLATEKDISLTSQTVTELPPHTVEGTSASLNDGSKTVLRSPHMNLSGTAESLNTVSITEYEEESLLTSFKLDTGAEDSSGSSPATSAIPFISENISQGYIFSSENPETITYDVLIPESARNASEDSTSSGSEESLKDPSMEGNVWFPSSTDITAQPDVGSGRESFLQTNYTEIRVDESEKTTKSFSAGPVMSQGPSVTDLEMPHYSTFAYFPTEVTPHAFTPSSRQQDLVSTVNVVYSQTTQPVYNGETPLQPSYSSEVFPLVTPLLLDNQILNTTPAASSSDSALHATPVFPSVDVSFESILSSYDGAPLLPFSSASFSSELFRHLHTVSQILPQVTSATESDKVPLHASLPVAGGDLLLEPSLAQYSDVLSTTHAASETLEFGSESGVLYKTLMFSQVEPPSSDAMMHARSSGPEPSYALSDNEGSQHIFTVSYSSAIPVHDSVGVTYQGSLFSGPSHIPIPKSSLITPTASLLQPTHALSGDGEWSGASSDSEFLLPDTDGLTALNISSPVSVAEFTYTTSVFGDDNKALSKSEIIYGNETELQIPSFNEMVYPSESTVMPNMYDNVNKLNASLQETSVSISSTKGMFPGSLAHTTTKVFDHEISQVPENNFSVQPTHTVSQASGDTSLKPVLSANSEPASSDPASSEMLSPSTQLLFYETSASFSTEVLLQPSFQASDVDTLLKTVLPAVPSDPILVETPKVDKISSTMLHLIVSNSASSENMLHSTSVPVFDVSPTSHMHSASLQGLTISYASEKYEPVLLKSESSHQVVPSLYSNDELFQTANLEINQAHPPKGRHVFATPVLSIDEPLNTLINKLIHSDEILTSTKSSVTGKVFAGIPTVASDTFVSTDHSVPIGNGHVAITAVSPHRDGSVTSTKLLFPSKATSELSHSAKSDAGLVGGGEDGDTDDDGDDDDDDRGSDGLSIHKCMSCSSYRESQEKVMNDSDTHENSLMDQNNPISYSLSENSEEDNRVTSVSSDSQTGMDRSPGKSPSANGLSQKHNDGKEENDIQTGSALLPLSPESKAWAVLTSDEESGSGQGTSDSLNENETSTDFSFADTNEKDADGILAAGDSEITPGFPQSPTSSVTSENSEVFHVSEAEASNSSHESRIGLAEGLESEKKAVIPLVIVSALTFICLVVLVGILIYWRKCFQTAHFYLEDSTSPRVISTPPTPIFPISDDVGAIPIKHFPKHVADLHASSGFTEEFETLKEFYQEVQSCTVDLGITADSSNHPDNKHKNRYINIVAYDHSRVKLAQLAEKDGKLTDYINANYVDGYNRPKAYIAAQGPLKSTAEDFWRMIWEHNVEVIVMITNLVEKGRRKCDQYWPADGSEEYGNFLVTQKSVQVLAYYTVRNFTLRNTKIKKGSQKGRPSGRVVTQYHYTQWPDMGVPEYSLPVLTFVRKAAYAKRHAVGPVVVHCSAGVGRTGTYIVLDSMLQQIQHEGTVNIFGFLKHIRSQRNYLVQTEEQYVFIHDTLVEAILSKETEVLDSHIHAYVNALLIPGPAGKTKLEKQFQLLSQSNIQQSDYSAALKQCNREKNRTSSIIPVERSRVGISSLSGEGTDYINASYIMGYYQSNEFIITQHPLLHTIKDFWRMIWDHNAQLVVMIPDGQNMAEDEFVYWPNKDEPINCESFKVTLMAEEHKCLSNEEKLIIQDFILEATQDDYVLEVRHFQCPKWPNPDSPISKTFELISVIKEEAANRDGPMIVHDEHGGVTAGTFCALTTLMHQLEKENSVDVYQVAKMINLMRPGVFADIEQYQFLYKVILSLVSTRQEENPSTSLDSNGAALPDGNIAESLESLVT

SEQ ID NO: 246 - protein, 536 aa, synthetic construct
MGSNKSKPKDASQRRRSLEPAENVHGAGGGAFPASQTPSKPASADGHRGPSAAFAPAAAEPKLFGGFNSSDTVTSPQRAGPLAGGVTTFVALYDYESRTETDLSFKKGERLQIVNNTEGDWWLAHSLSTGQTGYIPSNYVAPSDSIQAEEWYFGKITRRESERLLLNAENPRGTFLVRESETTKGAYCLSVSDFDNAKGLNVKHYKIRKLDSGGFYITSRTQFNSLQQLVAYYSKHADGLCHRLTTVCPTSKPQTQGLAKDAWEIPRESLRLEVKLGQGCFGEVWMGTWNGTTRVAIKTLKPGTMSPEAFLQEAQVMKKLRHEKLVQLYAVVSEEPIYIVTEYMSKGSLLDFLKGETGKYLRLPQLVDMAAQIASGMAYVERMNYVHRDLRAANILVGENLVCKVADFGLARLIEDNEYTARQGAKFPIKWTAPEAALYGRFTIKSDVWSFGILLTELTTKGRVPYPGMVNREVLDQVERGYRMPCPPECPESLHDLMCQCWRKEPEERPTFEYLQAFLEDYFTSTEPQYQPGENL

SEQ ID NO: 247 - protein, 509 aa, synthetic construct
MGCGCSSHPEDDWMENIDVCENCHYPIVPLDGKGTLLIRNGSEVRDPLVTYEGSNPPASPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFFTATEGQYQPQP

SEQ ID NO: 248 - protein, 537 aa, synthetic construct
MGCVQCKDKEATKLTEERDGSLNQSSGYRYGTDPTPQHYPSFGVTSIPNYNNFHAAGGQGLTVFGGVNSSSHTGTLRTRGGTGVTLFVALYDYEARTEDDLSFHKGEKFQILNSSEGDWWEARSLTTGETGYIPSNYVAPVDSIQAEEWYFGKLGRKDAERQLLSFGNPRGTFLIRESETTKGAYSLSIRDWDDMKGDHVKHYKIRKLDNGGYYITTRAQFETLQQLVQHYSERAAGLCCRLVVPCHKGMPRLTDLSVKTKDVWEIPRESLQLIKRLGNGQFGEVWMGTWNGNTKVAIKTLKPGTMSPESFLEEAQIMKKLKHDKLVQLYAVVSEEPIYIVTEYMNKGSLLDFLKDGEGRALKLPNLVDMAAQVAAGMAYIERMNYIHRDLRSANILVGNGLICKIADFGLARLIEDNEYTARQGAKFPIKWTAPEAALYGRFTIKSDVWSFGILLTELVTKGRVPYPGMNNREVLEQVERGYRMPCPQDCPISLHELMIHCWKKDPEERPTFEYLQSFLEDYFTATEPQYQPGENL

SEQ ID NO: 249 - protein, 543 aa, synthetic construct
MGCIKSKENKSPAIKYRPENTPEPVSTSVSHYGAEPTTVSPCPSSSAKGTAVNFSSLSMTPFGGSSGVTPFGGASSSFSVVPSSYPAGLTGGVTIFVALYDYEARTTEDLSFKKGERFQIINNTEGDWWEARSIATGKNGYIPSNYVAPADSIQAEEWYFGKMGRKDAERLLLNPGNQRGIFLVRESETTKGAYSLSIRDWDEIRGDNVKHYKIRKLDNGGYYITTRAQFDTLQKLVKHYTEHADGLCHKLTTVCPTVKPQTQGLAKDAWEIPRESLRLEVKLGQGCFGEVWMGTWNGTTKVAIKTLKPGTMMPEAFLQEAQIMKKLRHDKLVPLYAVVSEEPIYIVTEFMSKGSLLDFLKEGDGKYLKLPQLVDMAAQIADGMAYIERMNYIHRDLRAANILVGENLVCKIADFGLARLIEDNEYTARQGAKFPIKWTAPEAALYGRFTIKSDVWSFGILQTELVTKGRVPYPGMVNREVLEQVERGYRMPCPQGCPESLHELMNLCWKKDPDERPTFEYIQSFLEDYFTATEPQYQPGENL

SEQ ID NO: 250 - protein, 976 aa, synthetic construct
MELQAARACFALLWGCALAAAAAAQGKEVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSKVHEFQTLSPEGSGNLAVIGGVAVGVVLLLVLAGVGFFIHRRRKNQRARQSPEDVYFSKSEQLKPLKTYVDPHTYEDPNQAVLKFTTEIHPSCVTRQKVIGAGEFGEVYKGMLKTSSGKKEVPVAIKTLKAGYTEKQRVDFLGEAGIMGQFSHHNIIRLEGVISKYKPMMIITEYMENGALDKFLREKDGEFSVLQLVGMLRGIAAGMKYLANMNYVHRDLAARNILVNSNLVCKVSDFGLSRVLEDDPEATYTTSGGKIPIRWTAPEAISYRKFTSASDVWSFGIVMWEVMTYGERPYWELSNHEVMKAINDGFRLPTPMDCPSAIYQLMMQCWQQERARRPKFADIVSILDKLIRAPDSLKTLADFDPRVSIRLPSTSGSEGVPFRTVSEWLESIKMQQYTEHFMAAGYTAIEKVVQMTNDDIKRIGVRLPGHQKRIAYSLLGLKDQVNTVGIPI

SEQ ID NO: 251 - protein, 659 aa, synthetic construct
MAAVILESIFLKRSQQKKKTSPLNFKKRLFLLTVHKLSYYEYDFERGRRGSKKGSIDVEKITCVETVVPEKNPPPERQIPRRGEESSEMEQISIIERFPYPFQVVYDEGPLYVFSPTEELRKRWIHQLKNVIRYNSDLVQKYHPCFWIDGQYLCCSQTAKNAMGCQILENRNGSLKPGSSHRKTKKPLPPTPEEDQILKKPLPPEPAAAPVSTSELKKVVALYDYMPMNANDLQLRKGDEYFILEESNLPWWRARDKNGQEGYIPSNYVTEAEDSIEMYEWYSKHMTRSQAEQLLKQEGKEGGFIVRDSSKAGKYTVSVFAKSTGDPQGVIRHYVVCSTPQSQYYLAEKHLFSTIPELINYHQHNSAGLISRLKYPVSQQNKNAPSTAGLGYGSWEIDPKDLTFLKELGTGQFGVVKYGKWRGQYDVAIKMIKEGSMSEDEFIEEAKVMMNLSHEKLVQLYGVCTKQRPIFIITEYMANGCLLNYLREMRHRFQTQQLLEMCKDVCEAMEYLESKQFLHRDLAARNCLVNDQGVVKVSDFGLSRYVLDDEYTSSVGSKFPVRWSPPEVLMYSKFSSKSDIWAFGVLMWEIYSLGKMPYERFTNSETAEHIAQGLRLYRPHLASEKVYTIMYSCWHEKADERPTFKILLSNILDVMDEES

SEQ ID NO: 252 - DNA, 966 bp, synthetic construct; identical to SEQ ID NO: 5
  source 1..966: mol_type = other DNA

SEQ ID NO: 253 - protein, 321 aa, synthetic construct; identical to SEQ ID NO: 6

SEQ ID NO: 254 - DNA, 1218 bp, synthetic construct
  source 1..1218: mol_type = other DNA
atggagatggaaaaggagttcgagcagatcgacaagtccgggagctgggcggccatttaccaggatatccgacatgaagccagtgacttcccatgtagagtggccaagcttcctaagaacaaaaaccgaaataggtacagagacgtcagtccctttgaccatagtcggattaaactacatcaagaagataatgactatatcaacgctagtttgataaaaatggaagaagcccaaaggagttacattcttacccagggccctttgcctaacacatgcggtcacttttgggagatggtgtgggagcagaaaagcaggggtgtcgtcatgctcaacagagtgatggagaaaggttcgttaaaatgcgcacaatactggccacaaaaagaagaaaaagagatgatctttgaagacacaaatttgaaattaacattgatctctgaagatatcaagtcatattatacagtgcgacagctagaattggaaaaccttacaacccaagaaactcgagagatcttacatttccactataccacatggcctgactttggagtccctgaatcaccagcctcattcttgaactttcttttcaaagtccgagagtcagggtcactcagcccggagcacgggcccgttgtggtgcactgcagtgcaggcatcggcaggtctggaaccttctgtctggctgatacctgcctcttgctgatggacaagaggaaagacccttcttccgttgatatcaagaaagtgctgttagaaatgaggaagtttcggatggggctgatccagacagccgaccagctgcgcttctcctacctggctgtgatcgaaggtgccaaattcatcatgggggactcttccgtgcaggatcagtggaaggagctttcccacgaggacctggagcccccacccgagcatatccccccacctccccggccacccaaacgaatcctggagccacacaatgggaaatgcagggagttcttcccaaatcaccagtgggtgaaggaagagacccaggaggataaagactgccccatcaaggaagaaaaaggaagccccttaaatgccgcaccctacggcatcgaaagcatgagtcaagacactgaagttagaagtcgggtcgtggggggaagtcttcgaggtgcccaggctgcctccccagccaaaggggagccgtcactgcccgagaaggacgaggaccatgcactgagttactaa

SEQ ID NO: 255 - protein, 405 aa, synthetic construct
MEMEKEFEQIDKSGSWAAIYQDIRHEASDFPCRVAKLPKNKNRNRYRDVSPFDHSRIKLHQEDNDYINASLIKMEEAQRSYILTQGPLPNTCGHFWEMVWEQKSRGVVMLNRVMEKGSLKCAQYWPQKEEKEMIFEDTNLKLTLISEDIKSYYTVRQLELENLTTQETREILHFHYTTWPDFGVPESPASFLNFLFKVRESGSLSPEHGPVVVHCSAGIGRSGTFCLADTCLLLMDKRKDPSSVDIKKVLLEMRKFRMGLIQTADQLRFSYLAVIEGAKFIMGDSSVQDQWKELSHEDLEPPPEHIPPPPRPPKRILEPHNGKCREFFPNHQWVKEETQEDKDCPIKEEKGSPLNAAPYGIESMSQDTEVRSRVVGGSLRGAQAASPAKGEPSLPEKDEDHALSY

SEQ ID NO: 256 - DNA, 954 bp, synthetic construct
  source 1..954: mol_type = other DNA
atgcccaccaccatcgagcgggagttcgaagagttggatactcagcgtcgctggcagccgctgtacttggaaattcgaaatgagtcccatgactatcctcatagagtggccaagtttccagaaaacagaaatcgaaacagatacagagatgtaagcccatatgatcacagtcgtgttaaactgcaaaatgctgagaatgattatattaatgccagtttagttgacatagaagaggcacaaaggagttacatcttaacacagggtccacttcctaacacatgctgccatttctggcttatggtttggcagcagaagaccaaagcagttgtcatgctgaaccgcattgtggagaaagaatcggttaaatgtgcacagtactggccaacagatgaccaagagatgctgtttaaagaaacaggattcagtgtgaagctcttgtcagaagatgtgaagtcgtattatacagtacatctactacaattagaaaatatcaatagtggtgaaaccagaacaatatctcactttcattatactacctggccagattttggagtccctgaatcaccagcttcatttctcaatttcttgtttaaagtgagagaatctggctccttgaaccctgaccatgggcctgcggtgatccactgtagtgcaggcattgggcgctctggcaccttctctctggtagacacttgtcttgttttgatggaaaaaggagatgatattaacataaaacaagtgttactgaacatgagaaaataccgaatgggtcttattcagaccccagatcaactgagattctcatacatggctataatagaaggagcaaaatgtataaagggagattctagtatacagaaacgatggaaagaactttctaaggaagacttatctcctgcctttgatcattcaccaaacaaaataatgactgaaaaatacaatgggaacagataa

SEQ ID NO: 257 - protein, 317 aa, synthetic construct
MPTTIEREFEELDTQRRWQPLYLEIRNESHDYPHRVAKFPENRNRNRYRDVSPYDHSRVKLQNAENDYINASLVDIEEAQRSYILTQGPLPNTCCHFWLMVWQQKTKAVVMLNRIVEKESVKCAQYWPTDDQEMLFKETGFSVKLLSEDVKSYYTVHLLQLENINSGETRTISHFHYTTWPDFGVPESPASFLNFLFKVRESGSLNPDHGPAVIHCSAGIGRSGTFSLVDTCLVLMEKGDDINIKQVLLNMRKYRMGLIQTPDQLRFSYMAIIEGAKCIKGDSSIQKRWKELSKEDLSPAFDHSPNKIMTEKYNGNR

SEQ ID NO: 258 - DNA, 1164 bp, synthetic construct
  source 1..1164: mol_type = other DNA
atgcccaccaccatcgagcgggagttcgaagagttggatactcagcgtcgctggcagccgctgtacttggaaattcgaaatgagtcccatgactatcctcatagagtggccaagtttccagaaaacagaaatcgaaacagatacagagatgtaagcccatacgatcacagtcgtgttaaactgcaaaatgctgagaatgattatattaatgccagtttagttgacatagaagaggcacaaaggagttacatcttaacacagggtccacttcctaacacatgctgccatttctggcttatggtttggcagcagaagaccaaagcagttgtcatgctgaaccgcattgtggagaaagaatcggttaaatgtgcacagtactggccaacagatgaccaagagatgctgtttaaagaaacaggattcagtgtgaagctcttgtcagaagatgtgaagtcgtattatacagtacatctactacaattagaaaatatcaatagtggtgaaaccagaacaatatctcactttcattatactacctggccagattttggagtccctgaatcaccagcttcatttctcaatttcttgtttaaagtgagagaatctggctccttgaaccctgaccatgggcctgcggtgatccactgtagtgcaggcattgggcgctctggcaccttctctctggtagacacttgtcttgttttgatggaaaaaggagatgatattaacataaaacaagtgttactgaacatgagaaaataccgaatgggtcttattcagaccccagatcaactgagattctcatacatggctataatagaaggagcaaaatgtataaagggagattctagtatacagaaacgatggaaagaactttctaaggaagacttatctcctgcctttgatcattcaccaaacaaaataatgactgaaaaatacaatgggaacagaattggacttgaagaggaaaaactgaccggggacagatgcaccggactgtcgtcgaagatgcaagatactatggaagagaatagtgaatctgccttacgcaaacgtattagagaagatcgcaaggccactaccgcgcagaaagtccaacaaatgaaacaacggctgaacgaaaacgagagaaaaagaaagagaccacgtcttacagacacttaa

SEQ ID NO: 259 - protein, 387 aa, synthetic construct
MPTTIEREFEELDTQRRWQPLYLEIRNESHDYPHRVAKFPENRNRNRYRDVSPYDHSRVKLQNAENDYINASLVDIEEAQRSYILTQGPLPNTCCHFWLMVWQQKTKAVVMLNRIVEKESVKCAQYWPTDDQEMLFKETGFSVKLLSEDVKSYYTVHLLQLENINSGETRTISHFHYTTWPDFGVPESPASFLNFLFKVRESGSLNPDHGPAVIHCSAGIGRSGTFSLVDTCLVLMEKGDDINIKQVLLNMRKYRMGLIQTPDQLRFSYMAIIEGAKCIKGDSSIQKRWKELSKEDLSPAFDHSPNKIMTEKYNGNRIGLEEEKLTGDRCTGLSSKMQDTMEENSESALRKRIREDRKATTAQKVQQMKQRLNENERKRKRPRLTDT

SEQ ID NO: 260 - DNA, 921 bp, synthetic construct
  source 1..921: mol_type = other DNA
atggagcaagtggagatcctgaggaaattcatccagagggtccaggccatgaagagtcctgaccacaatggggaggacaacttcgcccgggacttcatgcggttaagaagattgtctaccaaatatagaacagaaaagatatatcccacagccactggagaaaaagaagacaatgttaaaaagaacagatacaaggacatactgccatttgatcacagccgagttaaattgacattaaagactccttcacaagattcagactatatcaatgcaaattttataaagggcgtctatgggccaaaagcatatgtagcaactcaaggacctttagcaaatacagtaatagatttttggaggatgatatgggagtataatgttgtgatcattgtaatggcctgccgagaatttgagatgggaaggaaaaaatgtgagcgctattggcctttgtatggagaagaccccataacgtttgcaccatttaaaatttcttgtgaggatgaacaagcaagaacagactacttcatcaggacactcttacttgaatttcaaaatgaatctcgtaggctgtatcagtttcattatgtgaactggccagaccatgatgttccttcatcatttgattctattctggacatgataagcttaatgaggaaatatcaagaacatgaagatgttcctatttgtattcattgcagtgcaggctgtggaagaacaggtgccatttgtgccatagattatacgtggaatttactaaaagctgggaaaataccagaggaatttaatgtatttaatttaatacaagaaatgagaacacaaaggcattctgcagtacaaacaaaggagcaatatgaacttgttcatagagctattgcccaactgtttgaaaaacagctacaactatatgaaattcatggagcttaa

SEQ ID NO: 261 - protein, 306 aa, synthetic construct
MEQVEILRKFIQRVQAMKSPDHNGEDNFARDFMRLRRLSTKYRTEKIYPTATGEKEDNVKKNRYKDILPFDHSRVKLTLKTPSQDSDYINANFIKGVYGPKAYVATQGPLANTVIDFWRMIWEYNVVIIVMACREFEMGRKKCERYWPLYGEDPITFAPFKISCEDEQARTDYFIRTLLLEFQNESRRLYQFHYVNWPDHDVPSSFDSILDMISLMRKYQEHEDVPICIHCSAGCGRTGAICAIDYTWNLLKAGKIPEEFNVFNLIQEMRTQRHSAVQTKEQYELVHRAIAQLFEKQLQLYEIHGA

SEQ ID NO: 262 - DNA, 900 bp, synthetic construct
  source 1..900: mol_type = other DNA
atgtcttctggtgtagatctgggtaccgagaacctgtacttccaatccatgtcccgtgtcctccaagcagaagagcttcatgaaaaggccctggaccctttcctgctgcaggcggaattctttgaaatccccatgaactttgtggatccgaaagagtacgacatccctgggctggtgcggaagaaccggtacaaaaccatacttcccaaccctcacagcagagtgtgtctgacctcaccagaccctgacgaccctctgagttcctacatcaatgccaactacatccggggctatggtggggaggagaaggtgtacatcgccactcagggacccatcgtcagcacggtcgccgacttctggcgcatggtgtggcaggagcacacgcccatcattgtcatgatcaccaacatcgaggagatgaacgagaaatgcaccgagtattggccggaggagcaggtggcgtacgacggtgttgagatcactgtgcagaaagtcattcacacggaggattaccggctgcgactcatctccctcaagagtgggactgaggagcgaggcctgaagcattactggttcacatcctggcccgaccagaagaccccagaccgggcccccccactcctgcacctggtgcgggaggtggaggaggcagcccagcaggaggggccccactgtgcccccatcatcgtccactgcagtgcagggattgggaggaccggctgcttcattgccaccagcatctgctgccagcagctgcggcaggagggtgtagtggacatcctgaagaccacgtgccagctccgtcaggacaggggcggcatgatccagacatgcgagcagtaccagtttgtgcaccacgtcatgagcctctacgaaaagcagctgtcccaccagtcctga

SEQ ID NO: 263 - protein, 299 aa, synthetic construct
MSSGVDLGTENLYFQSMSRVLQAEELHEKALDPFLLQAEFFEIPMNFVDPKEYDIPGLVRKNRYKTILPNPHSRVCLTSPDPDDPLSSYINANYIRGYGGEEKVYIATQGPIVSTVADFWRMVWQEHTPIIVMITNIEEMNEKCTEYWPEEQVAYDGVEITVQKVIHTEDYRLRLISLKSGTEERGLKHYWFTSWPDQKTPDRAPPLLHLVREVEEAAQQEGPHCAPIIVHCSAGIGRTGCFIATSICCQQLRQEGVVDILKTTCQLRQDRGGMIQTCEQYQFVHHVMSLYEKQLSHQS

SEQ ID NO: 264 - DNA, 885 bp, synthetic construct
  source 1..885: mol_type = other DNA
atggctgagaccacagataaagtcaaacaaggcttttgggaagaatttgagacactacaacaacaggagtgcaaacttctctacagccgaaaagagggtcaaaggcaagaaaacaaaaacaaaaatagatataaaaacatcctgccctttgatcataccagggttgtcctacacgatggtgatcccaatgagcctgtttcagattacatcaatgcaaatatcatcatgcctgaatttgaaaccaagtgcaacaattcaaagcccaaaaagagttacattgccacacaaggctgcctgcaaaacacggtgaatgacttttggcggatggtgttccaagaaaactcccgagtgattgtcatgacaacgaaagaagtggagagaggaaagagtaaatgtgtcaaatactggcctgatgagtatgctctaaaagaatatggcgtcatgcgtgttaggaacgtcaaagaaagcgccgctcatgactatacgctaagagaacttaaactttcaaaggttggacaagggaatacggagagaacggtctggcaataccactttcggacctggccggaccacggcgtgcccagcgaccctgggggcgtgctggacttcctggaggaggtgcaccataagcaggagagcatcatggatgcagggccggtcgtggtgcactgcagtgctggaattggccggacagggacgttcattgtgattgatattcttattgacatcatcagagagaaaggtgttgactgcgatattgacgttcccaaaaccatccagatggtgcggtctcagaggtcagggatggtccagacagaagcacagtaccgatttatctatatggcggtccagcattatattgaaacactacagcgcaggatttga

SEQ ID NO: 265 - protein, 294 aa, synthetic construct
MAETTDKVKQGFWEEFETLQQQECKLLYSRKEGQRQENKNKNRYKNILPFDHTRVVLHDGDPNEPVSDYINANIIMPEFETKCNNSKPKKSYIATQGCLQNTVNDFWRMVFQENSRVIVMTTKEVERGKSKCVKYWPDEYALKEYGVMRVRNVKESAAHDYTLRELKLSKVGQGNTERTVWQYHFRTWPDHGVPSDPGGVLDFLEEVHHKQESIMDAGPVVVHCSAGIGRTGTFIVIDILIDIIREKGVDCDIDVPKTIQMVRSQRSGMVQTEAQYRFIYMAVQHYIETLQRRI

SEQ ID NO: 266 - DNA, 1749 bp, synthetic construct
  source 1..1749: mol_type = other DNA
atggtgcaggagtcccgccccggctcggtccggtcgtattccgtgggctaccaggcccggtcgcggtcgtcgtcccagcgccgccattcgctcacgcggcagcgcagcagccagcggctcatccggacgatctccatcgagagcgatgtgagcaatatcacggacgatgatgatctgcgggcggtgaatgaaggggtggccggggtccagctcgacgtctccgagacggcgaacaaagggccacgccgggccagtgccaccgatgtcaccgactcgctgggctccacgtccagcgaatatatcgagatccccttcgtgaaagagacgctggacgcgagcctcccctcggattacctcaaacaagacatcctgaacctgatccaatccctgaagatctcgaaatggtacaataacaaaaagatccagcccgtcgcccaggacatgaacctcgtcaaaatctccggcgcgatgaccaatgcgatcttcaaggtggagtacccgaaactgccgtccctcctgctgcggatctatggcccgaatatcgataacatcatcgaccgcgaatatgaactccagatcctcgcgcggctctcgctgaaaaacatcgggccgtccctgtacggctgcttcgtgaatgggcgcttcgagcagttcctcgaaaactccaaaacgctgaccaaggatgatatccggaactggaaaaactcgcaacggatcgcccgccgcatgaaggagctgcatgtgggcgtgcccctcctctcgtcggagcggaagaatgggagcgcctgctggcaaaaaatcaaccaatggctccgcacgatcgagaaggtggatcagtgggtcggggacccgaagaacatcgagaacagcctcctctgcgaaaattggtccaaattcatggacatcgtcgatcggtaccacaagtggctgatcagccaagaacaagggatcgagcaagtcaacaaaaatctgatcttctgccataatgatgcccaatacgggaatctcctcttcaccgcgcccgtcatgaacaccccctccctgtataccgcgccgagctcgacctccctgacgtcccaaagcagcagcctcttcccctcgtccagcaacgtgatcgtcgatgatatcatcaatcccccgaagcaagaacaatcccaagattccaaactcgtggtcatcgatttcgaatacgccggggccaatcccgccgcgtacgatctcgccaatcacctctcggaatggatgtacgactataataacgccaaagccccgcaccagtgccacgccgaccggtaccccgacaaggagcaagtgctcaacttcctgtattcgtatgtcagccatctccgcggcggggccaaagagcccatcgatgaagaagtccagcgcctctataaatcgatcatccagtggcgccccacggtgcagctcttctggtcgctgtgggcgatcctgcaaagcggcaagctggaaaaaaaagaagccagcaccgccatcacccgcgaagaaatcgggcccaatgggaaaaagtatatcatcaagacggagcccgagtcgcccgaagaggacttcgtcgaaaatgacgacgaacccgaagccggcgtgtcgatcgataccttcgactacatggcctacgggcgggacaagatcgcggtgttctggggggacctgatcgggctgggcatcatcacggaggaggaatgcaagaacttctcgagcttcaaattcctcgacaccagctacctgtaa

SEQ ID NO: 267 - protein, 582 aa, synthetic construct
MVQESRPGSVRSYSVGYQARSRSSSQRRHSLTRQRSSQRLIRTISIESDVSNITDDDDLRAVNEGVAGVQLDVSETANKGPRRASATDVTDSLGSTSSEYIEIPFVKETLDASLPSDYLKQDILNLIQSLKISKWYNNKKIQPVAQDMNLVKISGAMTNAIFKVEYPKLPSLLLRIYGPNIDNIIDREYELQILARLSLKNIGPSLYGCFVNGRFEQFLENSKTLTKDDIRNWKNSQRIARRMKELHVGVPLLSSERKNGSACWQKINQWLRTIEKVDQWVGDPKNIENSLLCENWSKFMDIVDRYHKWLISQEQGIEQVNKNLIFCHNDAQYGNLLFTAPVMNTPSLYTAPSSTSLTSQSSSLFPSSSNVIVDDIINPPKQEQSQDSKLVVIDFEYAGANPAAYDLANHLSEWMYDYNNAKAPHQCHADRYPDKEQVLNFLYSYVSHLRGGAKEPIDEEVQRLYKSIIQWRPTVQLFWSLWAILQSGKLEKKEASTAITREEIGPNGKKYIIKTEPESPEEDFVENDDEPEAGVSIDTFDYMAYGRDKIAVFWGDLIGLGIITEEECKNFSSFKFLDTSYL

SEQ ID NO: 268 - DNA, 999 bp, synthetic construct
  source 1..999: mol_type = other DNA
atggaactcaatatcagcgaaagccggtcgcgcagcatccggtgcatcgtgaagctcgggggcgcggccatcacctgcaaaaacgaactcgaaaagatccatgacgaaaacctcgaagtggtggcctgccaactgcggcaagcgatgctggaaggctccgccccctccaaagtcatcggcatggactggtccaaacggccgggctcctccgaaatctcctgcgatgtggacgacatcggcgaccagaaatcgagcgaattctcgaagttcgtggtcgtccacggggcgggctcgttcggccatttccaagcgtcgcggtcgggcgtccataaagggggcctggagaagcccatcgtcaaagccgggttcgtcgcgacccggatctcggtcaccaatctcaatctcgagatcgtccgcgccctggcccgggaaggcatcccgacgatcgggatgagccccttcagctgcggctggtccacctccaaacgggatgtcgcgtcggcggatctcgcgacggtcgccaagaccatcgactcgggcttcgtgcccgtgctccatggggacgcggtcctggacaacatcctgggctgcacgatcctgtccggcgatgtcatcatccggcacctggccgaccatctcaagccggaatacgtcgtgttcctgaccgacgtgctcggggtctatgaccggcccccgagcccgtcggagccggacgccgtcctgctcaaggagatcgccgtcggggaggatggctcctggaaggtggtcaaccccctcctggaacataccgacaaaaaggtggattattccgtcgcggcccacgataccacgggggggatggaaaccaaaatctcggaagccgccatgatcgccaagctcggcgtggatgtgtacatcgtgaaagccgcgaccacccattcgcagcgggcgctcaatggcgacctgcgcgactccgtccccgaagactggctggggaccatcatccgcttcagcaaataa

SEQ ID NO: 269 - protein, 332 aa, synthetic construct
MELNISESRSRSIRCIVKLGGAAITCKNELEKIHDENLEVVACQLRQAMLEGSAPSKVIGMDWSKRPGSSEISCDVDDIGDQKSSEFSKFVVVHGAGSFGHFQASRSGVHKGGLEKPIVKAGFVATRISVTNLNLEIVRALAREGIPTIGMSPFSCGWSTSKRDVASADLATVAKTIDSGFVPVLHGDAVLDNILGCTILSGDVIIRHLADHLKPEYVVFLTDVLGVYDRPPSPSEPDAVLLKEIAVGEDGSWKVVNPLLEHTDKKVDYSVAAHDTTGGMETKISEAAMIAKLGVDVYIVKAATTHSQRALNGDLRDSVPEDWLGTIIRFSK

SEQ ID NO: 270 - DNA, 549 bp, synthetic construct
  source 1..549: mol_type = other DNA
atgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaataa

SEQ ID NO: 271 - protein, 182 aa, synthetic construct
MQTEHVILLNAQGVPTGTLEKYAAHTADTRLHLAFSSWLFNAKGQLLVTRRALSKKAWPGVWTNSVCGHPQLGESNEDAVIRRCRYELGVEITPPESIYPDFRYRATDPSGIVENEVCPVFAARTTSALQINDDEVMDYQWCDLADVLHGIDATPWAFSPWMVMQATNREARKRLSAFTQLK

SEQ ID NO: 272 - DNA, 294 bp, synthetic construct
  source 1..294: mol_type = other DNA
tggtactttggcaaacttgggcgtaaagatgcggagcgtcaacttctgtcctttggaaatccccgtggaaccttcttgatccgtgagtctgaaacggtcaagggcgcatatgctctgagtatccgcgactgggacgatatgaagggagatcacgtaaaacattatcttattcgcaagttggataacgggggatactacattacaacgcgcgcgcagtttgagaccttgcagcaactggttcagcactatagtgagcgtgccgcgggtctgagtagccgcttggtgcctccttcc

SEQ ID NO: 273 - protein, 98 aa, synthetic construct
WYFGKLGRKDAERQLLSFGNPRGTFLIRESETVKGAYALSIRDWDDMKGDHVKHYLIRKLDNGGYYITTRAQFETLQQLVQHYSERAAGLSSRLVPPS

SEQ ID NO: 274 - DNA, 291 bp, synthetic construct
  source 1..291: mol_type = other DNA
tggttcttccgcagtcaggggcgtaaggaggctgagcgtcagttattagccccgattaataaggccgggtcctttttgatccgcgagtcagagactgtcaaaggcgcgtttgccttaagtgtcaaagacgtcactacgcaaggcgagcttattaaacactatcttattcgttgcctggacgaaggagggtactacattagcccacgcattaccttccccagtttacaagcattagttcaacattactcaaagaaaggagacggtttatgtcagcgcttaacgttaccgtgc

SEQ ID NO: 275 - protein, 97 aa, synthetic construct
WFFRSQGRKEAERQLLAPINKAGSFLIRESETVKGAFALSVKDVTTQGELIKHYLIRCLDEGGYYISPRITFPSLQALVQHYSKKGDGLCQRLTLPC

SEQ ID NO: 276 - DNA, 267 bp, synthetic construct
  source 1..267: mol_type = other DNA
tggtatatgggtcccgtgagccgtcaggaagcacagacccgcttgcagggtcagcgtcacggcatgtttttagtgcgcgactctgaaacggtcaaaggtgactacgctttgtcagttagtgaaaatagccgtgtgtcgcattatttaattaactctttaccaaatcgtcgctttaaaatcggagatcaagagttcgatcacttacctgctttacttgaattttataaaatccattatcttgataccacgacactgatcgaaccagcg

SEQ ID NO: 277 - protein, 89 aa, synthetic construct
WYMGPVSRQEAQTRLQGQRHGMFLVRDSETVKGDYALSVSENSRVSHYLINSLPNRRFKIGDQEFDHLPALLEFYKIHYLDTTTLIEPA

SEQ ID NO: 278 - DNA, 312 bp, synthetic construct
  source 1..312: mol_type = other DNA
tggtatcacggacacatgtcccgtggtcaggcggaaacccttctgcaggcgaaaggcgagccctggaccttcttagtacgcgagagcgaaacagttaaaggggatttcgcattatcggtacttagtgaccagcctaaggccgggcctggaagccctttacgcgtaactcacattttagtaatgtgcgagggtggacgttataccgtcggcggattggagacgttcgatagccttaccgacttggttgagcatttcaaaaagactggcatcgaagaagcgtcaggagctttcgtttacttgcgtcagccgtat

SEQ ID NO: 279 - protein, 104 aa, synthetic construct
WYHGHMSRGQAETLLQAKGEPWTFLVRESETVKGDFALSVLSDQPKAGPGSPLRVTHILVMCEGGRYTVGGLETFDSLTDLVEHFKKTGIEEASGAFVYLRQPY

SEQ ID NO: 280 - DNA, 63 bp, synthetic construct
  source 1..63: mol_type = other DNA
tgcgaagacgccgatgaggacgaggatgactatcacaaccctggataccttgttgtgctgcct

SEQ ID NO: 281 - protein, 21 aa, synthetic construct
CEDADEDEDDYHNPGYLVVLP

SEQ ID NO: 282 - DNA, 63 bp, synthetic construct
  source 1..63: mol_type = other DNA
ggaggatggtcctccttcgaggaggacgactacgagtctcccaacgacgaccaagacggggag

SEQ ID NO: 283 - protein, 21 aa, synthetic construct
GGWSSFEEDDYESPNDDQDGE

SEQ ID NO: 284 - DNA, 51 bp, synthetic construct
  source 1..51: mol_type = other DNA
ccacaaccagacagtacagacaacgacgattacgatgacatttctgcagcg

SEQ ID NO: 285 - protein, 17 aa, synthetic construct
PQPDSTDNDDYDDISAA

SEQ ID NO: 286 - DNA, 33 bp, synthetic construct
  source 1..33: mol_type = other DNA
caaggcgtgatttactcagacttaaacctgcct

SEQ ID NO: 287 - protein, 11 aa, synthetic construct
QGVIYSDLNLP

SEQ ID NO: 288 - protein, 963 aa, synthetic construct
MAVAPRLFGGLCFRFRDQNPEVAVEGRLPISHSCVGCRRERTAMATVAANPAAAAAAVAAAAAVTEDREPQHEELPGLDSQWRQIENGESGRERPLRAGESWFLVEKHWYKQWEAYVQGGDQDSSTFPGCINNATLFQDEINWRLKEGLVEGEDYVLLPAAAWHYLVSWYGLEHGQPPIERKVIELPNIQKVEVYPVELLLVRHNDLGKSHTVQFSHTDSIGLVLRTARERFLVEPQEDTRLWAKNSEGSLDRLYDTHITVLDAALETGQLIIMETRKKDGTWPSAQLHVMNNNMSEEDEDFKGQPGICGLTNLGNTCFMNSALQCLSNVPQLTEYFLNNCYLEELNFRNPLGMKGEIAEAYADLVKQAWSGHHRSIVPHVFKNKVGHFASQFLGYQQHDSQELLSFLLDGLHEDLNRVKKKEYVELCDAAGRPDQEVAQEAWQNHKRRNDSVIVDTFHGLFKSTLVCPDCGNVSVTFDPFCYLSVPLPISHKRVLEVFFIPMDPRRKPEQHRLVVPKKGKISDLCVALSKHTGISPERMMVADVFSHRFYKLYQLEEPLSSILDRDDIFVYEVSGRIEAIEGSREDIVVPVYLRERTPARDYNNSYYGLMLFGHPLLVSVPRDRFTWEGLYNVLMYRLSRYVTKPNSDDEDDGDEKEDDEEDKDDVPGPSTGGSLRDPEPEQAGPSSGVTNRCPFLLDNCLGTSQWPPRRRRKQLFTLQTVNSNGTSDRTTSPEEVHAQPYIAIDWEPEMKKRYYDEVEAEGYVKHDCVGYVMKKAPVRLQECIELFTTVETLEKENPWYCPSCKQHQLATKKLDLWMLPEILIIHLKRFSYTKFSREKLDTLVEFPIRDLDFSEFVIQPQNESNPELYKYDLIAVSNHYGGMRDGHYTTFACNKDSGQWHYFDDNSVSPVNENQIESKAAYVLFYQRQDVARRLLSPAGSSGAPASPACSSPPSSEFMDVN

SEQ ID NO: 289 - protein, 494 aa, synthetic construct
MPLYSVTVKWGKEKFEGVELNTDEPPMVFKAQLFALTGVQPARQKVMVKGGTLKDDDWGNIKIKNGMTLLMMGSADALPEEPSAKTVFVEDMTEEQLASAMELPCGLTNLGNTCYMNATVQCIRSVPELKDALKRYAGALRASGEMASAQYITAALRDLFDSMDKTSSSIPPIILLQFLHMAFPQFAEKGEQGQYLQQDANECWIQMMRVLQQKLEAIEDDSVKETDSSSASAATPSKKKSLIDQFFGVEFETTMKCTESEEEEVTKGKENQLQLSCFINQEVKYLFTGLKLRLQEEITKQSPTLQRNALYIKSSKISRLPAYLTIQMVRFFYKEKESVNAKVLKDVKFPLMLDMYELCTPELQEKMVSFRSKFKDLEDKKVNQQPNTSDKKSSPQKEVKYEPFSFADDIGSNNCGYYDLQAVLTHQGRSSSSGHYVSWVKRKQDEWIKFDDDKVSIVTPEDILRLSGGGDWHIAYVLLYGPRRVEIMEEESEQ

SEQ ID NO: 290 - protein, 843 aa, synthetic construct
MTLDMDAVLSDFVRSTGAEPGLARDLLEGKNWDVNAALSDFEQLRQVHAGNLPPSFSEGSGGSRTPEKGFSDREPTRPPRPILQRQDDIVQEKRLSRGISHASSSIVSLARSHVSSNGGGGGSNEHPLEMPICAFQLPDLTVYNEDFRSFIERDLIEQSMLVALEQAGRLNWWVSVDPTSQRLLPLATTGDGNCLLHAASLGMWGFHDRDLMLRKALYALMEKGVEKEALKRRWRWQQTQQNKESGLVYTEDEWQKEWNELIKLASSEPRMHLGTNGANCGGVESSEEPVYESLEEFHVFVLAHVLRRPIVVVADTMLRDSGGEAFAPIPFGGIYLPLEVPASQCHRSPLVLAYDQAHFSALVSMEQKENTKEQAVIPLTDSEYKLLPLHFAVDPGKGWEWGKDDSDNVRLASVILSLEVKLHLLHSYMNVKWIPLSSDAQAPLAQPESPTASAGDEPRSTPESGDSDKESVGSSSTSNEGGRRKEKSKRDREKDKKRADSVANKLGSFGKTLGSKLKKNMGGLMHSKGSKPGGVGTGLGGSSGTETLEKKKKNSLKSWKGGKEEAAGDGPVSEKPPAESVGNGGSKYSQEVMQSLSILRTAMQGEGKFIFVGTLKMGHRHQYQEEMIQRYLSDAEERFLAEQKQKEAERKIMNGGIGGGPPPAKKPEPDAREEQPTGPPAESRAMAFSTGYPGDFTIPRPSGGGVHCQEPRRQLAGGPCVGGLPPYATFPRQCPPGRPYPHQDSIPSLEPGSHSKDGLHRGALLPPPYRVADSYSNGYREPPEPDGWAGGLRGLPPTQTKCKQPNCSFYGHPETNNFCSCCYREELRRREREPDGELLVHRF

SEQ ID NO: 291 - protein, 9 aa, synthetic construct
QGPHHHHHH

SEQ ID NO: 292 - DNA, 1641 bp, synthetic construct
  source 1..1641: mol_type = other DNA
atggccctgaccgaagagaaaccgatccgcccgatcgctaacttcccgccgtctatctggggtgaccagttcctgatctacgaaaagcaggttgagcagggtgttgaacagatcgtaaacgacctgaagaaagaagttcgtcagctgctgaaagaagctctggacatcccgatgaaacacgctaacctgttgaagctgatcgacgagatccagcgtctgggtatcccgtaccacttcgaacgcgaaatcgaccacgcactgcagtgcatctacgaaacctacggcgacaactggaacggcgaccgttcttctctgtggtttcgtctgatgcgtaaacagggctactacgttacctgtgacgtttttaacaactacaaggacaagaacggtgctttcaaacagtctctggctaacgacgttgaaggcctgctggaactgtacgaagcgacctccatgcgtgtaccgggtgaaatcatcctggaggacgcgctgggtttcacccgttctcgtctgtccattatgactaaagacgctttctctactaacccggctctgttcaccgaaatccagcgtgctctgaaacagccgctgtggaaacgtctgccgcgtatcgaagcagcacagtacattccgttttaccagcagcaggactctcacaacaagaccctgctgaaactggctaagctggaattcaacctgctgcagtctctgcacaaagaagaactgtctcacgtttgtaagtggtggaaggcatttgacatcaagaaaaacgcgccgtgcctgcgtgaccgtatcgttgaatgttacttctggggtctgggttctggttatgaaccacagtactcccgtgcacgtgtgttcttcactaaagctgtagctgttatcaccctgatcgatgacacttacgatgcttacggcacctacgaagaactgaagatctttactgaagctgtagaacgctggtctatcacttgcctggacactctgccggagtacatgaaaccgatctacaaactgttcatggatacctacaccgaaatggaggaattcctggcaaaagaaggccgtaccgacctgttcaactgcggtaaagagtttgttaaagaattcgtacgtaacctgatggttgaagctaaatgggctaacgaaggccatatcccgactaccgaagaacatgacccggttgttatcatcaccggcggtgcaaacctgctgaccaccacttgctatctgggtatgtccgacatctttaccaaggaatctgttgaatgggctgtttctgcaccgccgctgttccgttactccggtattctgggtcgtcgtctgaacgacctgatgacccacaaagcagagcaggaacgtaaacactcttcctcctctctggaatcctacatgaaggaatataacgttaacgaggagtacgcacagactctgatctataaagaagttgaagacgtatggaaagacatcaaccgtgaatacctgactactaaaaacatcccgcgcccgctgctgatggcagtaatctacctgtgccagttcctggaagtacagtacgctggtaaagataacttcactcgcatgggcgacgaatacaaacacctgatcaaatccctgctggtttacccgatgtccatctga

SEQ ID NO: 293 - protein, 546 aa, synthetic construct; identical to SEQ ID NO: 4

SEQ ID NO: 294 - DNA, 1782 bp, synthetic construct; identical to SEQ ID NO: 20
  source 1..1782: mol_type = other DNA

SEQ ID NO: 295 - protein, 593 aa, synthetic construct; identical to SEQ ID NO: 7

SEQ ID NO: 296 - DNA, 2409 bp, synthetic construct; identical to SEQ ID NO: 12
  source 1..2409: mol_type = other DNA

SEQ ID NO: 297 - protein, 802 aa, synthetic construct
MSSSTGTSKVVSETSSTIVDDIPRLSANYHGDLWHHNVIQTLETPFRESSTYQERADELVVKIKDMFNALGDGDISPSAYDTAWVARLATISSDGSEKPRFPQALNWVFNNQLQDGSWGIESHFSLCDRLLNTTNSVIALSVWKTGHSQVQQGAEFIAENLRLLNEEDELSPDFQIIFPALLQKAKALGINLPYDLPFIKYLSTTREARLTDVSAAADNIPANMLNALEGLEEVIDWNKIMRFQSKDGSFLSSPASTACVLMNTGDEKCFTFLNNLLDKFGGCVPCMYSIDLLERLSLVDNIEHLGIGRHFKQEIKGALDYVYRHWSERGIGWGRDSLVPDLNTTALGLRTLRMHGYNVSSDVLNNFKDENGRFFSSAGQTHVELRSVVNLFRASDLAFPDERAMDDARKFAEPYLREALATKISTNTKLFKEIEYVVEYPWHMSIPRLEARSYIDSYDDNYVWQRKTLYRMPSLSNSKCLELAKLDFNIVQSLHQEELKLLTRWWKESGMADINFTRHRVAEVYFSSATFEPEYSATRIAFTKIGCLQVLFDDMADIFATLDELKSFTEGVKRWDTSLLHEIPECMQTCFKVWFKLMEEVNNDVVKVQGRDMLAHIRKPWELYFNCYVQEREWLEAGYIPTFEEYLKTYAISVGLGPCTLQPILLMGELVKDDVVEKVHYPSNMFELVSLSWRLTNDTKTYQAEKARGQQASGIACYMKDNPGATEEDAIKHICRVVDRALKEASFEYFKPSNDIPMGCKSFIFNLRLCVQIFYKFIDGYGIANEEIKDYIRKVYIDPIQV

SEQ ID NO: 298 - DNA, 1641 bp, synthetic construct; identical to SEQ ID NO: 292
  source 1..1641: mol_type = other DNA